(19) United States
(12) Patent Application Publication
(10) Pub. No.: US 2012/0077781 A1
(43) Pub. Date: Mar. 29, 2012

(54) FLAVIN DERIVATIVES

(76) Inventors: Robert Gadwood, Portage, MI (US); Harpreet Kaur, Brampton (CA); Kevin Kells, Cambridge (CA); Jeffrey A. Leiby, Kalamazoo, MI (US); David Moon, Brampton (CA); Vinh Pham, Markham (CA); Philip D.G. Coish, North Haven, CT (US); Kenneth F. Blount, Guilford, CT (US); Brian R. Dixon, Woodbridge, CT (US); Jayhyuk Myung, Woodbridge, CT (US); David Osterman, Glastonbury, CT (US); Stephanie Avola, Woodbridge (CA); Nick Baboulas, Scarborough (CA); Angelica Bello, North York (CA); Judd Berman, Toronto (CA); Bruce R. Evans, Holland, MI (US); Tan Quach, Toronto (CA); Heinrich J. Schostarez, Portage, MI (US); Frank C. Sciavolino, Waterford, CT (US); Dennis Underwood, Jamaica Plain, MA (US); Phil Wickens, Richmond Hill (CA); Jeffrey Wilson, Toronto (CA); Justin Wu, Markham (CA)

(21) Appl. No.: 12/737,781

(22) PCT Filed: Aug. 11, 2009
(86) PCT No.: PCT/US09/04576

§ 371 Date: Nov. 9, 2011

Related U.S. Application Data
(60) Provisional application No. 61/188,619, filed on Aug. 11, 2008.
(60) Provisional application No. 61/211,314, filed on Mar. 25, 2009.

(51) Int. Cl. A61K 31/675; A61P 31/04; C07D 487/04; C07F 9/6561; A61K 31/525; A61K 31/5377

(52) U.S. Cl. 514/81; 514/251; 514/232.8; 544/251; 544/244; 544/115

(57) ABSTRACT

The present invention relates novel flavin derivatives and other flavin derivatives, their use and compositions for use as riboswitch ligands and/or anti-infectives. The invention also provides method of making novel flavin derivatives.

### RELATED APPLICATIONS

This application claims priority from U.S. Provisional Application No. 61/188,619, filed Aug. 11, 2008, and U.S. Provisional Application No. 61/211,314, filed Mar. 25, 2009, the contents of each of which are incorporated by reference in their entirety.

### TECHNICAL FIELD

The present invention relates to flavin derivatives and their use and compositions for use as riboswitch ligands and/or anti-infectives. The invention also provides methods of making novel flavin derivatives.

### BACKGROUND OF THE INVENTION

The fast growing rate of antibiotic resistance over the past decades has raised serious concerns that the antibiotic treatment options currently available will soon be ineffective. With the widespread usage of antibiotics in combination with the rapid growing rate of bacterial resistance in stark contrast with the decade-old chemical scaffolds available for their treatment, it is imperative that new drugs are developed in the battle against bacterial pathogens.

In many bacteria and fungi, RNA structures termed riboswitches regulate the expression of various genes crucial for survival or virulence. Typically located within the 5′-untranslated region (5′-UTR) of certain mRNAs, members of each known class of riboswitch can fold into a distinct, three-dimensionally structured receptor that recognizes a specific organic metabolite. When the cognate metabolite is present at sufficiently high concentrations during transcription of the mRNA, the riboswitch receptor binds to the metabolite and induces a structural change in the nascent mRNA that prevents expression of the open reading frame (ORF), thereby altering gene expression. In the absence of the cognate metabolite, the riboswitch folds into a structure that does not interfere with the expression of the ORF.

Sixteen different classes of riboswitches have been reported. Members of each class of riboswitch bind to the same metabolite and share a highly conserved sequence and secondary structure. Riboswitch motifs have been identified that bind to thiamine pyrophosphate (TPP), flavin mononucleotide (FMN), glycine, guanine, 3′-5′-cyclic eiguanylic acid (c-di-GMP), molybdenum cofactor, glucosamine-6-phosphate (GlcN6P), lysine, adenine, and adocobalamin (AdoCbl) riboswitches. Additionally, four distinct riboswitch motifs have been identified that recognize S-adenosylmethionine (SAM) I, II and III, IV and two distinct motifs that recognize pre-queosine-1 (PreQ1). Several antimetabolite ligands have also been identified that bind to known riboswitch classes, including pyrithiamine pyrophosphate (PTPP) which binds TPP riboswitches, L-aminoethylcysteine (AEC) and DL-4-oxalysine which bind to lysine riboswitches and roseoflavin and FMN which bind to FMN riboswitches. The riboswitch-receptors bind to their respective ligands in an interface that approaches the level of complexity and selectivity of proteins. This highly specific interaction allows riboswitches to discriminate against most intimately related analogs of ligands. For instance, the receptor of a guanine-binding riboswitch from Bacillus subtilis forms a three-dimensional structure such that the ligand is almost completely enveloped. The guanine is positioned between two aromatic bases and each polar functional group of the guanine hydrogen bonds with four additional riboswitch nucleotides surrounding it. This level of specificity allows the riboswitch to discriminate against most closely related purine analogs. Similarly, studies of the SAM-binding riboswitches reveal that nearly every functional group of SAM is critical in binding the ligands, allowing it to discriminate highly similar compounds such as S-adenosylhomocysteine (SAH) and S-adenosylmethionine (SAM), which only differ by a single methyl group. Likewise, TPP riboswitches comprise one subdomain that recognizes every polar functional group of the 4-amino-5-hydroxymethyl-2-methylpyrimidine (HMP) moiety, albeit not the thiazole moiety, and another subdomain that coordinates two metal ions and several water molecules to bind the negatively charged pyrophosphate moiety of the ligand. Similar to TPP, guanine and SAM riboswitches, FMN riboswitches form receptor structures that are highly specific for the natural metabolite FMN. It is by this highly specific interaction that allows for the design of small molecules for the regulation of specific genes.

FMN riboswitches are of particular interest of this invention because it is believed that the riboswitch binds to flavin mono-nucleotide (FMN) and represses the expression of enzymes responsible for riboflavin and FMN biosynthesis. Riboflavin is a water-soluble vitamin that is converted by flavokinases and FAD synthases to co-factors FMN and FAD, which are indispensable cofactors involved in energy metabolism and metabolism of fats, ketones, carbohydrates and proteins crucial for all living organisms. Although vertebrates rely on uptake of vitamin from their gut for riboflavin sources, most prokaryotes, fungi and plants synthesize the necessary riboflavin for survival. It is therefore suggested that compounds that are selective for FMN riboswitch may be useful targets against bacterial pathogens in shutting down biosynthesis of riboflavin crucial for survival or virulence. In addition, no examples of the FMN, TPP, nor any other riboswitch class have presently been identified in humans. Therefore, riboswitches appear to offer the potential for the discovery of selective antipathogenic drugs. It is therefore the objective of this invention to provide novel flavin derivatives for targeting FMN riboswitches and methods of treating infections comprising administering flavin derivatives.

### SUMMARY OF THE INVENTION

The current invention relates to a compound of formula I:

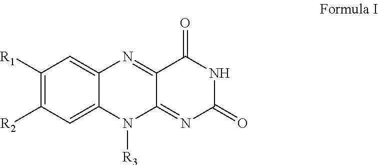

wherein
(i) R₁ is H, C₁₋₈ alkyl (e.g., methyl) or C₃₋₇ cycloalkyl;
(ii) R₂ is H, halo (e.g., chloro), C₁₋₈alkyl (e.g., methyl or ethyl), C₁₋₈alkoxy (e.g., methoxy or ethoxy), —N(R₄)(R₅), C₃₋₇cycloalkyl or C₄₋₇heterocycle (e.g., piperazinyl or pyrrolidinyl) wherein said heterocycle is optionally substituted with C₁₋₈alkyl (e.g., 4-methyl-piperazin-1-yl) or hydroxyC₁₋₈alkyl (e.g., 4-hydroxyethyl-piperazin-1-yl);
(iii) R₃ is H or C₁₋₈ alkyl (e.g., n-butyl, n-pentyl, n-propyl, n-hexyl or n-heptyl), wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇); or
(iv) R₁ and R₂ are connected so as to form a cyclic ring structure optionally containing one or more heteroatoms selected from N, O and S (e.g., —OCH₂CH₂O—);
(v) R₄ and R₅ are independently selected from H, C₃₋₇ cycloalkyl (e.g., cyclopropyl or cyclopentyl), C₄₋₇heterocycle (e.g., piperazinyl), and C₁₋₈alkyl (e.g., methyl or ethyl) wherein said alkyl is optionally substituted with one or more groups selected from —OR₁₁, —C(O)OR₉, —N(R₆)(R₇) (e.g., amino or dimethylamino), C₁₋₈alkoxyl (e.g., methoxy), C₆₋₁₀aryl (e.g., phenyl), C₅₋₁₀ heteroaryl (e.g., pyridinyl) wherein said aryl or heteroaryl are optionally substituted with halo (e.g., 4-fluorophenyl), and C₄₋₇heterocycle wherein said heterocycle is optionally substituted with C₁₋₈alkyl (e.g., morpholin-4-yl or 4-methylpiperazin-1-yl);
(vi) R₆ and R₇ are independently selected from H, C₁₋₈alkyl (e.g., methyl or ethyl), —C₁₋₈alkyl-OR₁₁, —C(O)OR₉, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈allyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl and aryl wherein said aryl is optionally substituted with —COOR₉;
(vii) R₈ is H, C₁₋₈alkyl (e.g., methyl, ethyl or t-butyl), —OR₁₁ or —OBn;
(viii) R₉ and R₁₇ are independently selected from H, C₁₋₈alkyl (e.g., methyl, ethyl or t-butyl), —C₁₋₄alkyl-OC(O)R₁₂, phenyl and Bn wherein said phenyl and Bn are optionally substituted with one or more halo or C₁₋₄alkoxy (e.g., 3-chloro-phenylmethyl, 3-fluoro-phenylmethyl, 4-methoxy-3-fluorophenylmethyl);
(ix) R₁₀ is H, C₁₋₈alkyl (e.g., methyl or ethyl), —C₁₋₈alkyl-OR₁₁, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl, or aryl wherein said aryl is optionally substituted with —COOR₉, or —C₁₋₄alkyl-OC(O)R₁₂;
(x)₁₁ is H, or —C₁₋₄alkyl-OC(O)R₁₂ (e.g., —CH₂—OC(O)R₁₂);
(xi) R₁₂ is C₁₋₈alkyl (e.g., methyl, ethyl, t-Butyl) or —OC₁₋₈alkyl (e.g., methoxy, ethoxy, t-butoxy);
(xii) R₁₃, R₁₄, R₁₅ and R₁₆ are independently selected from H, C₁₋₈alkyl, and —C₁₋₈alkyl-COOR₁₈ (e.g., —CH(methyl)-COOH, —CH(isopropyl)-COOH, —CH(isobutyl)-COOH, —CH(sec-butyl)-COOH), wherein the alkyl group of C₁₋₈alkyl-COOR₁₈ is optionally substituted with hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH); and
(xiii) R₁₈ is H or C₁₋₈alkyl;
in free, salt or prodrug form.

The invention further relates to a compound of Formula I as follows:
1.1 a Compound of Formula I, wherein R₁ is H, C₁₋₈alkyl (e.g., methyl) or C₃₋₇cycloalkyl;
1.2 a Compound of Formula I or 1.1, wherein R₁ is H,
1.3 a Compound of Formula I or 1.1, wherein R₁ is C₃₋₇cycloalkyl;
1.4 a Compound of Formula I or 1.1, wherein R₁ is C₁₋₈allyl;
1.5 a Compound of Formula I or 1.1 or 1.4, wherein R₁ is methyl;
1.6 a Compound of Formula I or any of 1.1-1.5, R₂ is H, halo (e.g., chloro), C₁₋₈alkyl (e.g., methyl or ethyl), C₁₋₈alkoxy (e.g., methoxy or ethoxy), —N(R₄)(R₅), C₃₋₇cycloalkyl or C₄₋₇heterocycle (e.g., piperazinyl or pyrrolidinyl) wherein said heterocycle is optionally substituted with C₁₋₈alkyl (e.g., 4-methyl-piperazin-1-yl) or hydroxyC₁₋₈alkyl (e.g., hydroxyethylpiperazin-1-yl);
1.7 a Compound of Formula I or any of 1.1-1.6, wherein R₂ is C₁₋₈alkyl (e.g., methyl or ethyl);
1.8 a Compound of Formula I or any of 1.1-1.7, wherein R₂ is methyl;
1.9 a Compound of Formula I or any of 1.1-1.7, wherein R₂ is ethyl;
1.10 a Compound of Formula I or any of 1.1-1.6, wherein R₂ is C₁₋₈alkoxy;
1.11 a Compound of Formula I or any of 1.1-1.6 or 1.10, wherein R₂ is methoxy;
1.12 a Compound of Formula I or any of 1.1-1.6 or 1.10, wherein R₂ is ethoxy;
1.13 a Compound of Formula I or any of 1.1-1.6, wherein R₂ is C₃₋₇ cycloalkyl;
1.14 a Compound of Formula I or any of 1.1-1.6, wherein R₂ is —N(R₄)(R₅) and R₄ and R₅ are independently selected from H, C₃₋₇ cycloalkyl (e.g., cyclopropyl or cyclopentyl), C₄₋₇ heterocycle (e.g., piperazinyl), and C₁₋₈alkyl (e.g., methyl or ethyl) wherein said alkyl is optionally substituted with one or more groups selected from —OR₁₁, —C(O)OR₉, —N(R₆)(R₇) (e.g., amino or dimethylamino), C₁₋₈alkoxyl (e.g., methoxy), C₆₋₁₀aryl (e.g., phenyl), C₅₋₁₀heteroaryl (e.g., pyridinyl) wherein said aryl or heteroaryl are optionally substituted with halo (e.g., 4-fluorophenyl), and C₄₋₇heterocycle wherein said heterocycle is optionally substituted with C₁₋₈alkyl (e.g., morpholin-4-yl or 4-methylpiperazin-1-yl);
1.15 Formula 1.14, wherein either R₄ or R₅ is H,
1.16 Formula 1.14, wherein either R₄ or R₅ is C₁₋₈alkyl (e.g., methyl or ethyl);
1.17 Formula 1.14 or 1.16, wherein either R₄ or R₅ is methyl;
1.18 Formula 1.14 or 1.16, wherein either R₄ or R₅ is ethyl;
1.19 Formula 1.14, wherein either R₄ or R₅ is C₃₋₇cycloalkyl (e.g., cyclopropyl or cyclopentyl);
1.20 Formula 1.14 or 1.19, wherein either R₄ or R₅ is cyclopropyl or cyclopentyl;
1.21 Formula 1.14, wherein either R₄ or R₅ is C₄₋₇ heterocycle (e.g., piperazinyl);
1.22 Formula 1.14 or 1.21, wherein either R₄ or R₅ is piperazinyl;
1.23 Formula 1.14, wherein either R₄ or R₅ is C₁₋₈alkyl (e.g., methyl or ethyl) wherein said alkyl is substituted with C₄₋₇heterocycle, which heterocycle is optionally substituted with C₁₋₈alkyl (e.g., morpholin-4-yl or 4-methylpiperazin-1-yl);
1.24 Formula 1.14 or 1.23, wherein either R₄ or R₅ is C₁₋₈alkyl (e.g., methyl or ethyl) wherein said alkyl is substituted with C₄₋₇ heterocycle;
1.25 Formula 1.14, 1.23 or 1.24, wherein either R₄ or R₅ is C₁₋₈alkyl (e.g., methyl or ethyl) wherein said alkyl is substituted with morpholinyl;
1.26 Formula 1.14, 1.23, 1.24 or 1.25, wherein either R₄ or R₅ is —CH₂CH₂— morpholine;
1.27 Formula 1.14 or 1.23, wherein either R₄ or R₅ is C₁₋₈alkyl (e.g., methyl or ethyl) wherein said alkyl is substituted with C₄₋₇heterocycle, which C₄₋₇heterocycle is substituted with C₁₋₈alkyl (e.g., 4-methylpiperazin-1-yl);
1.28 Formula 1.14, 1.23 or 1.27, wherein either R₄ or R₅ is C₁₋₈alkyl (e.g., methyl or ethyl) wherein said alkyl is substituted with 4-methylpiperazin-1-yl;
1.29 Formula 1.14, 1.23, 1.27 or 1.28, wherein either R₄ or R₅ is —CH₂CH₂— (4-methylpiperazin-1-yl);
1.30 Formula 1.14, wherein either R₄ or R₅ is C₁₋₈alkyl (e.g., methyl or ethyl) wherein said alkyl is substituted with —C(O)OR₉;
1.31 Formula 1.14 or 1.30, wherein either R₄ or R₅ is —CH₂CH₂C(O)OR₉ and R₉ is H, C₁₋₈alkyl (e.g., methyl, ethyl or t-butyl), —C₁₋₄alkyl-OC(O)R₁₂, phenyl and Bn wherein said phenyl and Bn are optionally substituted with halo or C₁₋₄alkoxy (3-chloro-phenylmethyl, 3-fluoro-phenylmethyl, 4-methoxy-3-fluorophenylmethyl), and R₁₂ is as hereinbefore described in Formula I;
1.32 Formula 1.31, wherein either R₄ or R₅ is —CH₂CH₂C(O)OH;
1.33 Formula 1.31, wherein either R₄ or R₅ is —CH₂CH₂C(O)O-(tert-butyl);
1.34 Formula 1.31, wherein either R₄ or R₅ is —C₁₋₈ alkyl-C(O)O—C₁₋₄alkyl-OC(O)R₁₂ (e.g., —CH₂CH₂C(O)OCH₂OC(O)R₁₂—CH₂CH₂C(O)OCH₂—OC(O)R₁₂);
1.35 Formula 1.14, wherein either R₄ or R₅ is C₁₋₈alkyl (e.g., methyl or ethyl) wherein said alkyl is substituted with C₆₋₁₀aryl (e.g., phenyl) or C₅₋₁₀ heteroaryl (e.g., pyridinyl) which aryl and heteroaryl is optionally substituted with halo;
1.36 Formula 1.14 or 1.35, wherein either R₄ or R₅ is C₁₋₈alkyl (e.g., methyl or ethyl) wherein said alkyl is substituted with phenyl which phenyl is optionally substituted with halo (e.g., fluoro);
1.37 Formula 1.14, 1.35 or 1.36, wherein either R₄ or R₅ is —CH₂CH₂-(4-fluorophenyl);
1.38 Formula 1.14 or 1.35, wherein either R₄ or R₅ is C₁₋₈alkyl (e.g., methyl or ethyl) wherein said alkyl is substituted with C₅₋₁₀ heteroaryl (e.g., pyridinyl);
1.39 Formula 1.14 or 1.35, wherein either R₄ or R₅ is —CH₂CH₂— (pyridin-2-yl);
1.40 Formula 1.14, wherein either R₄ or R₅ is C₁₋₈alkyl wherein said alkyl is substituted with —OR₁₁ wherein R₁₁ is H or —C₁₋₄alkyl-OC(O)R₁₂;
1.41 Formula 1.14 or 1.40, wherein either R₄ or R₅ is hydroxyethyl;
1.42 Formula 1.14 or 1.40, wherein either R₄ or R₅ is -ethyl-O—C₁₋₄alkyl-OC(O)R_{12;}
1.43 Formula 1.14, wherein either R₄ or R₅ is C₁₋₈alkyl wherein said alkyl is substituted with C₁₋₆alkoxyl (e.g., methoxy);
1.44 Formula 1.14 or 1.43, wherein either R₄ or R₅ is methoxyethyl;
1.45 Formula 1.14, wherein either R₄ or R₅ is C₁₋₈alkyl (e.g., methyl or ethyl) wherein said alkyl is substituted with —N(R₆)(R₇) and wherein R₆ and R₇ are independently selected from H, C₁₋₈alkyl (e.g., methyl or ethyl), —C₁₋₈alkyl-OR₁₁, —C(O)OR₉, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl and aryl wherein said aryl is optionally substituted with —COOR₉;
1.46 Formula 1.14, wherein either R₄ or R₅ is C₁₋₈alkyl (e.g., methyl or ethyl) wherein said alkyl is substituted with —N(R₆)(R₇) and R₆ and R₇ are independently selected from H, C₁₋₈alkyl, and C(O)OR₉;
1.47 Formula 1.45 or 1.46 or 1.47, wherein either R₆ or R₇ is H;
1.48 Formula 1.45 or 1.46, wherein either R₆ or R₇ is C₁₋₈alkyl (e.g., methyl);
1.49 Formula 1.45 or 1.46 or 1.48, wherein either R₆ or R₇ is methyl;
1.50 Formula 1.45 or 1.46, wherein either R₆ or R₇ is —C₁₋₈alkyl-OR₁₁;
1.51 Formula 1.45 or 1.50, wherein either R₆ or R₇ is —CH₂CH₂OH;
1.52 Formula 1.45, wherein either R₆ or R₇ is —C₁₋₈ alkyl-C(O)O—C₁₋₄alkyl-OC(O)R_{12;}
1.53 Formula 1.45 or 1.46, wherein either R₆ or R₇ is —C₁₋₈alkyl-C(O)OH,
1.54 Formula 1.45, 1.46 or 1.53, wherein either R₆ or R₇ is —CH₂CH₂C(O)OH;
1.55 Formula 1.45 or 1.46, wherein either R₆ or R₇ is —C₁₋₈alkyl-C(O)OR₉ and R₉ is H, C₁₋₈alkyl (e.g., methyl, ethyl or t-butyl), —C₁₋₄alkyl-OC(O)R₁₂, phenyl and Bn wherein said phenyl and Bn are optionally substituted with one or more halo or C₁₋₄alkoxy (e.g., 3-chloro-phenylmethyl, 3-fluoro-phenylmethyl, 4-methoxy-3-fluorophenylmethyl);
1.56 Formula 1.45, 1.46 or 1.55, wherein either R₆ or R₇ is —C₁₋₈alkyl-C(O)OC₁₋₄alkyl-OC(O)R₁₂;
1.57 Formula 1.45, 1.46 or 1.55, wherein either R₆ or R₇ is —CH₂CH₂CH₂—C(O)O—C₁₋₄alkyl-OC(O)R₁₂;
1.58 Formula 1.45, 1.46 or 1.55, wherein either R₆ or R₇ is —CH₂CH₂C(O)O-tert-butyl;
1.59 Formula 1.45 or 1.46, wherein either R₆ or R₇ is —CH₂CH₂CH₂C(O)O-tert-butyl;
1.60 Formula 1.45, wherein either R₆ or R₇ is —C₁₋₈ alkyl-C(O)N(H)R₈;
1.61 Formula 1.45 or 1.60, wherein either R₆ or R₇ is —(CH₂)₃—C(O)N(H)R₈;
1.62 Formula 1.61, wherein R₈ is —OBn;
1.63 Formula 1.61, wherein R₈ is —OR₁₁;
1.64 Formula 1.45, wherein either R₆ or R₇ is C₁₋₈ alkyl-P(O)(OR₉)(OR₁₇) and R₉ and R₁₇ are independently selected from H, C₁₋₈alkyl (e.g., methyl, ethyl or t-butyl), —C₁₋₄alkyl-OC(O)R₁₂, phenyl and Bn wherein said phenyl and Bn are optionally substituted with one or more halo or C₁₋₄alkoxy (e.g., 3-chloro-phenylmethyl, 3-fluoro-phenylmethyl, 4-methoxy-3-fluorophenylmethyl;
1.65 Formula 1.45 or 1.64 wherein either R₆ or R₇ is —CH₂CH₂—P(O)(OH)₂;
1.66 Formula 1.45 or 1.64 wherein either R₆ or R₇ is —CH₂CH₂—P(O)(O—C₁₋₈alkyl)₂;
1.67 Formula 1.45 or 1.64 wherein either R₆ or R₇ is —CH₂CH₂—P(O)(OH)(O—C₁₋₈alkyl);
1.68 Formula 1.45, wherein either R₆ or R₇ is —C₁₋₈alkyl-P(O)(OR₉)(NR₁₃R₁₄) and R₉, R₁₃, R₁₄, are as hereinbefore described in Formula I;
1.69 Formula 1.45 or 1.64 wherein either R₆ or R₇ is —CH₂CH₂—P(O)(O-phenyl)(N(H)CH(CH₃)COOH);
1.70 Formula 1.45 or 1.64 wherein either R₆ or R₇ is —CH₂CH₂—P(O)(OH)(N(H)CH(CH₃)COOH);
1.71 Formula 1.45 or 1.64 wherein either R₆ or R₇ is —C₁₋₈alkyl-P(O)(NR₁₃R₁₄)(NR₁₅R₁₆);
1.72 Formula 1.45 or 1.64 wherein either R₆ or R₇ is —CH₂CH₂-1)(O)(N(F)CH(CH₃)COOH)₂;
1.73 Formula 1.45, wherein either R₆ or R₇ is —C₁₋₈alkyl-OP(O)(OR₉)(OR₁₇) and R₉ and R₁₇ are independently selected from H, C₁₋₈alkyl (e.g., methyl, ethyl or t-butyl), —C₁₋₄alkyl-OC(O)R₁₂, phenyl and Bn wherein phenyl and Bn are optionally substituted with halo or C₁₋₄alkoxy (e.g., 3-chloro-phenylmethyl, 3-fluoro-phenylmethyl, 4-methoxy-3-fluorophenylmethyl);
1.74 Formula 1.45 or 1.73 wherein either R₆ or R₇ is —CH₂CH₂—OP(O)(OH)₂;
1.75 Formula 1.45 or 1.73 wherein either R₆ or R₇ is —CH₂CH₂—OP(O)(O—C₁₋₈alkyl)₂;
1.76 Formula 1.45 or 1.73 wherein either R₆ or R₇ is —CH₂CH₂—OP(O)(OH)(O—C₁₋₈alkyl);
1.77 Formula 1.45, wherein either R₆ or R₇ is —C₁₋₈alkyl-OP(O)(OR₉)(NR₁₃R₁₄) and R₉, R₁₃, R₁₄, are as hereinbefore described in Formula I;
1.78 Formula 1.45 or 1.77 wherein either R₆ or R₇ is —CH₂CH₂—OP(O)(O-phenyl)(N(H)CH(CH₃)COOH);
1.79 Formula 1.45 or 1.77 wherein either R₆ or R₇ is —CH₂CH₂—OP(O)(OH)(N(H)CH(CH₃)COOH);
1.80 Formula 1.45 wherein either R₆ or R₇ is —C₁₋₈alkyl-OP(O)(NR₁₃R₁₄)(NR₁₅R₁₆);
1.81 Formula 1.45 or 1.80 wherein either R₆ or R₇ is —CH₂CH₂—OP(O)(N(H)CH(CH₃)COOH)₂;
1.82 Formula 1.45, wherein either R₆ or R₇ is —C₁₋₈alkyl-N(H)—S(O)₂(CF₃);
1.83 Formula 1.45 or 1.82, wherein either R₆ or R₇ is —(CH₂)₃—N(H)—S(O)₂(CF₃);
1.84 Formula 1.45, wherein either R₆ or R₇ is 7,8-dimethyl-isoalloxazin-10-yl-ethyl;
1.85 Formula I, or any of 1.1-1.6 or 1.14, wherein R₂ is —NH₂;
1.86 Formula I, or any of 1.1-1.6 or 1.14, wherein R₂ is methylamino;
1.87 Formula I or any of 1.1-1.6 or 1.14, wherein R₂ is dimethylamino;
1.88 Formula I or any of 1.1-1.6 or 1.14, wherein R₂ is diethylamino;
1.89 Formula I or any of 1.1-1.6 or 1.14, wherein R₂ is —N(H)—CH₂CH₂-(morpholin-4-yl);
1.90 Formula I or any of 1.1-1.6 or 1.14, wherein R₂ is —N(H)—CH₂CH₂-(4-methyl-piperazin-1-yl);
1.91 Formula I or any of 1.1-1.6 or 1.14, wherein R₂ is —N(H)—CH₂CH₂OH;
1.92 Formula I or any of 1.1-1.6 or 1.14, wherein R₂ is —N(H)—CH₂CH₂OCH₃;
1.93 Formula I or any of 1.1-1.6 or 1.14, wherein R₂ is —N(H)CH₂CH₂—N(H)—C(O)O-tert-butyl;
1.94 Formula I or any of 1.1-1.6 or 1.14, wherein R₂ is —N(H)-cyclopropyl;
1.95 Formula I or any of 1.1-1.6 or 1.14, wherein R₂ is 4-methylpiperazin-1-yl;
1.96 Formula I or any of 1.1-1.6 or 1.14, wherein R₂ is —N(H)CH₂CH₂-(4-fluorophenyl);
1.97 Formula I or any of 1.1-1.6 or 1.14, wherein R₂ is —N(H)CH₂CH₂-(pyridine-2-yl);
1.98 Formula I or any of 1.1-1.6 or 1.14, wherein R₂ is —N(H)CH₂CH₂NH₂;
1.99 Formula I or any of 1.1-1.6 or 1.14, wherein R₂ is —N(H)CH₂CH₂CH₂NH₂,
1.100 Formula I or any of 1.1-1.6 or 1.14, wherein R₂ is —N(H)CH₂CH₂N(CH₃)₂;
1.101 Formula I or any of 1.1-1.6 or 1.14, wherein R₂ is —N(H)—CH₂CH₂COOH;
1.102 Formula I or any of 1.1-1.6 or 1.14, wherein R₂ is —N(H)—CH₂CH₂COO-tert-butyl;
   1.103 Formula I or any of 1.1-1.6, wherein R₂ is halo;
   1.104 Formula I or any of 1.1-1.6, wherein R₂ is chloro;
   1.105 Formula I or any of 1.1-1.6, wherein R₂ is C₄₋₇ heterocycle (e.g., piperazinyl or pyrrolidinyl) optionally substituted with C₁₋₈alkyl (e.g., 4-methyl-piperazin-1-yl) or hydroxyC₁₋₈alkyl (e.g., hydroxyethyl);
   1.106 Formula I or any of 1.1-1.6, wherein R₂ is 4-(hydroxyethyl)piperazin-1-yl;
   1.107 Formula I or any of 1.1-1.6, wherein R₂ is pyrrolidin-1-yl;
   1.108 Formula I or any of 1.1-1.6, wherein R₁ and R₂ are connected so as to form a cyclic ring structure optionally containing one or more heteroatoms selected from N, O and S (e.g., —OCH₂CH₂O—);
   1.109 Any of the foregoing formulae, wherein R₃ is H or C₁₋₈alkyl (e.g., n-butyl, n-pentyl, n-propyl, n-hexyl or n-heptyl), wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇); wherein:
      R₆ and R₇ are independently selected from H, C₁₋₈alkyl (e.g., methyl or ethyl), —C₁₋₈alkyl-OR₁₁, —C(O)OR₉, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl and aryl wherein said aryl is optionally substituted with —COOR₉;
      R₈ is H, C₁₋₈ alkyl (e.g., methyl or t-butyl), —OR₁₁ or —OBn;
      R₉ and R₁₇ are independently selected from H, C₁₋₈alkyl (e.g., methyl, ethyl or t-butyl), —C₁₋₄alkyl-OC(O)R₁₂, phenyl and Bn wherein said phenyl and Bn are optionally substituted with one or more halo or C₁₋₄alkoxy (e.g., 3-chloro-phenylmethyl, 3-fluoro-phenylmethyl, 4-methoxy-3-fluorophenylmethyl);
      R₁₀ is H, C₁₋₈alkyl (e.g., methyl or ethyl), —C₁₋₈alkyl-OR₁₁, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl, or aryl wherein said aryl is optionally substituted with —COOR₉, or —C₁₋₄alkyl-OC(O)R₁₂;
      R₁₁ is H or —C₁₋₄alkyl-OC(O)R₁₂ (e.g., —CH₂—OC(O)R₁₂);
      R₁₂ is C₁₋₈alkyl (e.g., methyl, ethyl, t-Butyl) or —OC₁₋₈alkyl (e.g., methoxy, ethoxy, t-butoxy);
      R₁₃, R₁₄, R₁₅ and R₁₆ are independently selected from H, C₁₋₈alkyl, and —C₁₋₈alkyl-COOR₁₈ (e.g., —CH(methyl)-COOH, —CH(isopropyl)-COOH, —CH(isobutyl)-COOH, —CH(sec-butyl)-COOH), wherein the alkyl group of C₁₋₈alkyl-COOR₁₈ is optionally substituted with hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH);
      R₁₈ is H or C₁₋₈alkyl;
   1.110 Any of 1.1-1.109, wherein R₃ is C₁₋₈alkyl (e.g., n-butyl, n-pentyl, n-propyl, n-hexyl or n-heptyl);
   1.111 Any of 1.1-1.109, wherein R₃ is n-butyl;
   1.112 Any of 1.1-1.109, wherein R₃ is C₁₋₈alkyl (e.g., n-butyl, n-pentyl, n-propyl, n-hexyl or n-heptyl), wherein the alkyl group is optionally substituted with one or more —OR₁₀ and R₁₀ is H, C₁₋₈alkyl (e.g., methyl or ethyl), —C₁₋₈alkyl-OR₁₁, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl, or aryl wherein said aryl is optionally substituted with —COOR₉, or —C₁₋₄alkyl-OC(O)R₁₂ and R₈, R₉, R₁₁-R₁₈ are as hereinbefore described in Formula I;
   1.113 Formula 1.112, wherein R₁₀ is H,
   1.114 Formula 1.112, wherein R₁₀ is C₁₋₈alkyl (e.g., methyl, ethyl or propyl);
   1.115 Formula 1.112, wherein R₁₀ is —C₁₋₈alkyl-OR₁₁;
   1.116 Formula 1.112, wherein R₁₀ is —C₁₋₈alkyl-C(O)OR₉;
   1.117 Formula 1.112, wherein R₁₀ is —C₁₋₈alkyl(amine)-C(O)OR₉ (e.g., —CH₂CH₂CH(NH₂)COOH);
   1.118 Formula 1.112, wherein R₁₀ is —C₁₋₈alkyl-C(O)N(H)R₈;
   1.119 Formula 1.112, wherein R₁₀ is —C₁₋₈alkyl-P(O)(OR₉)(OR₁₇);
   1.120 Formula 1.112, wherein R₁₀ is —C₁₋₈alkyl-P(O)(OR₉)(NR₁₃R₁₄);
   1.121 Formula 1.112, wherein R₁₀ is —C₁₋₈alkyl-P(O)(NR₁₃R₁₄)(NR₁₅R₁₆);
   1.122 Formula 1.112, wherein R₁₀ is —C₁₋₈alkyl-OP(O)(OR₉)(OR₁₇);
   1.123 Formula 1.112, wherein R₁₀ is —C₁₋₈alkyl-OP(O)(OR₉)(NR₁₃R₁₄);
   1.124 Formula 1.112, wherein R₁₀ is —C₁₋₈alkyl-OP(O)(NR₁₃R₁₄)(NR₁₅R₁₆);
   1.125 Formula 1.112, wherein R₁₀ is —C₁₋₈alkyl-N(H)—S(O)₂(CF₃);
   1.126 Formula 1.112, wherein R₁₀ is 7,8-dimethyl-isoalloxazin-10-yl-ethyl;
   1.127 Formula 1.112, wherein R₁₀ is aryl optionally substituted with —COOR₉;
   1.128 Formula 1.112, wherein R₁₀ is —C₁₋₄alkyl-OC(O)R₁₂;
   1.129 Any of formulae 1.112-1.128, wherein R₃ is C₁₋₈alkyl further substituted with at least one —OR₁₀ group, wherein R₁₀ is H (e.g., polyhydroxylated);
   1.130 Any of 1.1-1.109, wherein R₃ is C₁₋₈alkyl (e.g., n-butyl, n-pentyl, n-propyl, n-hexyl or n-heptyl), wherein the alkyl group is optionally substituted with —N(R₆)(R₇), wherein R₆ and R₇ are described in any one of Formulae 1.47-1.84;
   1.131 Any of 1.1-1.109, wherein R₃ is C₁₋₈alkyl (e.g., n-butyl, n-pentyl, n-propyl, n-hexyl or n-heptyl), wherein the alkyl group is optionally substituted with —C(O)N(R₆)(R₇), wherein R₆ and R₇ are described in any one of Formulae 1.47-1.84;
   1.132 Any of 1.1-1.109, wherein R₃ is C₁₋₈alkyl (e.g., n-butyl, n-pentyl, n-propyl, n-hexyl or n-heptyl), wherein the alkyl group is optionally substituted with —OP(O)(OR₉)(OR₁₇);
   1.133 Formula 1.132 wherein R₃ is —C₁₋₈alkyl-OP(O)(OH)₂;
   1.134 Formula 1.132 wherein R₃ is —C₁₋₈alkyl-OP(O)(O—C₁₋₈alkyl)₂;
   1.135 Formula 1.132 wherein R₃ is —CH₂CH₂—OP(O)(OH)(O—C₁₋₈alkyl);
   1.136 Formula 1.132, wherein R₃ is —(CH₂)₄—OP(O)(OR₉)(OR₁₇), —(CH₂)₅—OP(O)(OR₉)(OR₁₇), —(CH₂)₆—OP(O)(OR₉)(OR₁₇) or —(CH₂)₇—OP(O)(OR₉)(OR₁₇);
   1.137 Any of 1.1-1.109, wherein R₃ is C₁₋₈alkyl (e.g., n-butyl, n-pentyl, n-propyl, n-hexyl or n-heptyl), wherein the alkyl group is optionally substituted with —OP(O)(OR₉)(NR₁₃R₁₄);
   1.138 Formula 1.137 wherein R₃ is —C₁₋₈alkyl-OP(O)(O-phenyl)(N(H)CH(CH₃)COOH);
   1.139 Formula 1.137 wherein R₃ is —C₁₋₈alkyl-OP(O)(OH)(N(H)CH(CH₃)COOH);
   1.140 Formula 1.137 wherein R₃ is —C₁₋₈alkyl-OP(O)(O—C₁₋₈alkyl)(N(H)CH(CH₃)COOH);
   1.141 Formula 1.137, wherein R₃ is —(CH₂)₄—OP(O)(OR₉)(NR₁₃R₁₄), —(CH₂)₅—OP(O)(OR₉)₁₃R₁₄), —(CH₂)₆—OP(O)(OR₉)(NR₁₃R₁₄) or —(CH₂)₇—OP(O)(OR₉)(NR₁₃R₁₄);
   1.142 Any of 1.1-1.109, wherein R₃ is C₁₋₈alkyl (e.g., n-butyl, n-pentyl, n-propyl, n-hexyl or n-heptyl), wherein the alkyl group is optionally substituted with OP(O)(NR₁₃R₁₄)(NR₁₅R₁₆);
   1.143 Formula 1.142, wherein R₃ is —C₁₋₈alkyl-OP(O)(N(H)CH(CH₃)COOH)₂;
   1.144 Formula 1.142, wherein R₃ is —(CH₂)₄—OP(O)(NR₁₃R₁₄)(NR₁₅R₁₆), —(CH₂)₅—OP(O)(NR₁₃R₁₄)(NR₁₅R₁₆), —(CH₂)₆—OP(O)(NR₁₃R₁₄)(NR₁₅R₁₆) or —(CH₂)₇—OP(O)(NR₁₃R₁₄)(NR₁₅R₁₆);
   1.145 Any of 1.1-1.109, wherein R₃ is C₁₋₈alkyl (e.g., n-butyl, n-pentyl, n-propyl, n-hexyl or n-heptyl), wherein the alkyl group is optionally substituted with —P(O)(OR₉)(OR₁₇);
   1.146 Formula 1.145 wherein R₃ is —C₁₋₈alkyl-P(O)(OH)₂;
   1.147 Formula 1.145 wherein R₃ is —C₁₋₈alkyl-P(O)(O—C₁₋₈alkyl)₂;
   1.148 Formula 1.145 wherein R₃ is —C₁₋₈alkyl-P(O)(OH)(O—C₁₋₈alkyl);
   1.149 Formula 1.145, wherein R₃ is —(CH₂)₄—P(O)(OR₉)(OR₁₇), —(CH₂)₅—P(O)(OR₉)(OR₁₇), —(CH₂)₆—P(O)(OR₉)(OR₁₇) or —(CH₂)₇—P(O)(OR₉)(OR₁₇);
   1.150 Any of 1.1-1.109, wherein R₃ is C₁₋₈alkyl (e.g., n-butyl, n-pentyl, n-propyl, n-hexyl or n-heptyl), wherein the alkyl group is optionally substituted with —P(O)(OR₉)(NR₁₃R₁₄);
   1.151 Formula 1.150 wherein R₃ is —C₁₋₈alkyl-P(O)(O-phenyl)(N(H)CH(CH₃)COOH);
   1.152 Formula 1.150 wherein R₃ is —C₁₋₈alkyl-P(O)(OH)(N(H)CH(CH₃)COOH);
   1.153 Formula 1.150, wherein R₃ is —(CH₂)₄—P(O)(OR₉)(NR₁₃R₁₄), —(CH₂)₅—P(O)(OR₉)(NR₁₃R₁₄), —(CH₂)₆—P(O)(OR₉)(NR₁₃R₁₄) or —(CH₂)₇—P(O)(OR₉)(NR₁₃R₁₄);
   1.154 Any of 1.1-1.109, wherein R₃ is C₁₋₈alkyl (e.g., n-butyl, n-pentyl, n-propyl, n-hexyl or n-heptyl), wherein the alkyl group is optionally substituted with —P(O)(NR₁₃R₁₄)(NR₁₅R₁₆);
   1.155 Formula 1.154 wherein R₃ is —C₁₋₈alkyl-P(O)(N(H)CH(CH₃)COOH)₂;
   1.156 Formula 1.154, wherein R₃ is —(CH₂)₄⁻¹³(O)(NR₁₃R₁₄)(NR₁₅R₁₆), —(CH₂)₅—P(O)(NR₁₃R₁₄)(NR₁₅R₁₆), —(CH₂)₆—P(O)(NR₁₃R₁₄)(NR₁₅R₁₆) or —(CH₂)₇—P(O)(NR₁₃R₁₄)(NR₁₅R₁₆);
   1.157 Formula I or any of 1.1-1.109, wherein R₃ is C₁₋₈alkyl optionally substituted —C(O)OR₉;
   1.158 Formula I or any of 1.1-1.109, wherein R₃ is C₁₋₈alkyl substituted with one or more —OR₁₀;
   1.159 Any of the preceding formulae, wherein R₈ is H, C₁₋₈alkyl (e.g., methyl, ethyl or t-butyl), —OR₁₁ or —OBn;
   1.160 Any of the preceding formulae, wherein R₉ and R₁₇ are independently selected from H, C₁₋₈alkyl (e.g., methyl, ethyl or t-butyl), —C₁₋₄alkyl-OC(O)R₁₂, phenyl and Bn wherein said phenyl and Bn are optionally substituted with one or more halo or C₁₋₄alkoxy (e.g., 3-chloro-phenylmethyl, 3-fluoro-phenylmethyl, 4-methoxy-3-fluorophenylmethyl);
   1.161 Any of the preceding formulae, wherein R₉ and R₁₇ are independently C₁₋₈alkyl (e.g., methyl, ethyl or t-butyl);
   1.162 Any of the preceding formulae, wherein R₉ and R₁₇ are independently H,
   1.163 Any of formulae 1.1-1.160, wherein R₉ and R₁₇ are independently —C₁₋₄alkyl-OC(O)R₁₂;
   1.164 Any of formulae 1.1-1.160, wherein R₉ and R₁₇ are independently phenyl or Bn wherein phenyl and Bn are optionally substituted with halo or C₁₋₄alkoxy (e.g., 3-chloro-phenylmethyl, 3-fluoro-phenylmethyl, 4-methoxy-3-fluorophenylmethyl);
   1.165 Any of formulae 1.1-1.160, wherein R₉ and R₁₇ are independently phenyl;
   1.166 Any of the preceding formulae wherein R₁₁ is H or —C₁₋₄alkyl-OC(O)R_{12;}
   1.167 Any of the preceding formulae wherein R₁₂ is C₁₋₈alkyl (e.g., methyl, ethyl, t-Butyl) or —OC₁₋₈alkyl (e.g., methoxy, ethoxy, t-butoxyl);
   1.168 Any of the preceding formulae wherein R₁₃, R₁₄, R₁₅ and R₁₆ are independently selected from H, C₁₋₈alkyl, and —C₁₋₈alkyl-COOR₁₈ (e.g., —CH(methyl)-COOH, —CH(isopropyl)-COOH, —CH(isobutyl)-COOH, —CH(sec-butyl)-COOH), wherein the alkyl group of C₁₋₈alkyl-COOR₁₈ is optionally substituted with hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH);
   1.169 Any of formulae 1.1-1.168 wherein R₁₃, R₁₄, R₁₅ and R₁₆ are independently H or —CH(CH₃)COOH;
   1.170 Any of the preceding formulae wherein R₃ is further substituted with —OR₁₀);
   1.171 Any of formulae 1.1-1.169 wherein R₁₈ is H,
   1.172 Any of formulae 1.1-1.169 wherein R₁₈ is C₁₋₈allyl;
   1.173 Formula I or any of 1.1-1.109 wherein R₃ is H,
   1.174 Formula I or any of 1.1-1.109, wherein R₃ is C₁₋₈alkyl (e.g., n-butyl, n-pentyl, n-propyl, n-hexyl or n-heptyl);
   1.175 Formula I or any of 1.1-1.109, wherein R₃ is n-butyl;
   1.176 Formula I or any of 1.1-1.109, wherein R₃ is —(CH₂)₅OH;
   1.177 Formula I or any of 1.1-1.109, wherein R₃ is —(CH₂)₄OH;
   1.178 Formula I or any of 1.1-1.109, wherein R₃ is —(CH₂)₃OH;
   1.179 Formula I or any of 1.1-1.109, wherein R₃ is (2S,3S,4R)-2,3,4,5-tetrahydroxypentyl;
   1.180 Formula I or any of 1.1-1.109, wherein R₃ is —(CH₂)₅OP(O)(OH)₂;
   1.181 Formula I or any of 1.1-1.109, wherein R₃ is 5-dihydrogenphosphate-(2S,3S,4R)-2,3,4-trihydroxypentyl;
   1.182 Formula I or any of 1.1-1.109, wherein R₃ is 5-phosphonate-(2S,3S,4R)-2,3,4-trihydroxypentyl;
   1.183 Formula I or any of 1.1-1.109, wherein R₃ is —(CH₂)₄C(O)OH,
   1.184 Formula I or any of 1.1-1.109, wherein R₃ is —(CH₂)₅C(O)OH,
   1.185 Formula I or any of 1.1-1.109, wherein R₃ is —(CH₂)₆C(O)OH;
   1.186 Formula I or any of 1.1-1.109, wherein R₃ is —(CH₂)₇C(O)OH,
   1.187 Formula 1 or any of 1.1-1.109, wherein R₃ is —(CH₂)₆C(O)OCH₃;
   1.188 Formula I or any of 1.1-1.109, wherein R₃ is —(CH₂)₆C(O)N(H)OH;
   1.189 Formula I or any of 1.1-1.109, wherein R₃ is CH₂CH₂N(H)CH₂CH₂CH₂—C(O)OH;
   1.190 Formula I or any of 1.1-1.109, wherein R₃ is CH₂CH₂N(H)CH₂CH₂CH₂—C(O)O-tert-butyl;
   1.191 Formula I or any of 1.1-1.109, wherein R₃ is —CH₂CH₂N(H)—(3-carboxyphenyl);
   1.192 Formula I or any of 1.1-1.109, wherein R₃ is —CH₂CH₂N(H)—CH₂CH₂CH(NH₂)COOH;
   1.193 Formula I or any of 1.1-1.109, wherein R₃ is —CH₂CH₂N(H)CH₂CH₂—P(O)(OH)₂;
   1.194 Formula I or any of 1.1-1.109, wherein R₃ is —(CH₂)₆P(O)(OH)₂;
   1.195 Formula I or any of 1.1-1.109, wherein R₃ is —(CH₂)₆P(O)(O-phenyl)(N(H)CH(CH₃)COOH);
   1.196 Formula I or any of 1.1-1.109, wherein R₃ is —(CH₂)₆P(O)(O-phenyl)(N(H)CH(CH₃)COO—C₁₋₈alkyl);
   1.197 Formula I or any of 1.1-1.109, wherein R₃ is —CH₂CH₂N(H)—CH₂CH₂CH₂C(O)N(H)—OBn;
   1.198 Formula I or any of 1.1-1.109, wherein R₃ is —CH₂CH₂N(H)—CH₂CH₂CH₂—C(O)N(H)(OR₉) (e.g., —CH₂CH₂N(H)—CH₂CH₂CH₂—C(O)N(H)(OH), —CH₂CH₂N(H)—CH₂CH₂CH₂—C(O)N(H)(O—CH₂—O—C(O)-^{t}Butyl or —CH₂CH₂N(H)—CH₂CH₂CH₂—C(O)N(H)(O—CH₂—O—C(O)—O-^{t}Butyl);
   1.199 Formula I or any of 1.1-1.109, wherein R₃ is —(CH₂)₆—C(O)N(H)(OH);
   1.200 Formula I or any of 1.1-1.109, wherein R₃ is —CH₂CH₂N(H)CH₂CH₂CH₂—N(H)—S(O)₂CF₃;
   1.201 Formula I or any of 1.1-1.109, wherein R₃ is C₁₋₈alkyl (e.g., n-butyl, n-pentyl, n-propyl, n-hexyl or n-heptyl), wherein the alkyl group is optionally substituted with one or more —CN;
   1.202 Formula I or any of 1.1-1.109, wherein R₃ is —(CH₂)₄—CN;
   1.203 any of the preceding formulae wherein the Compound of Formula I is selected from any of the following:

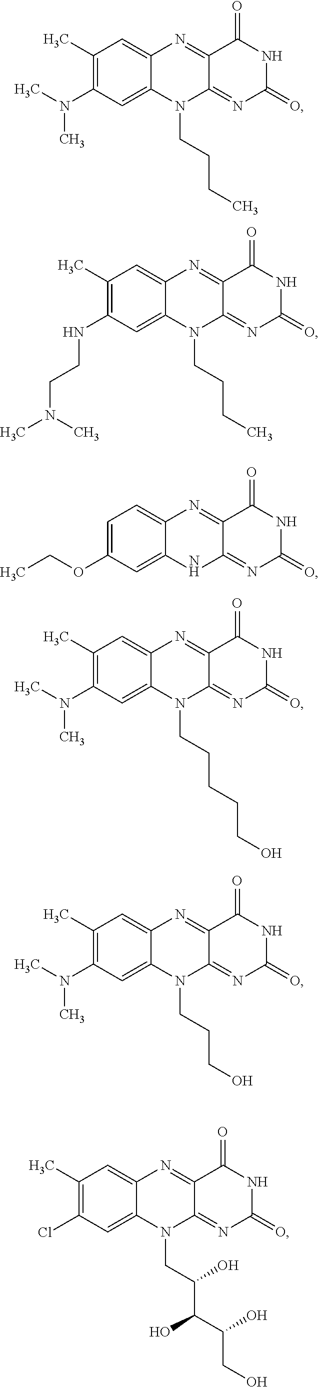

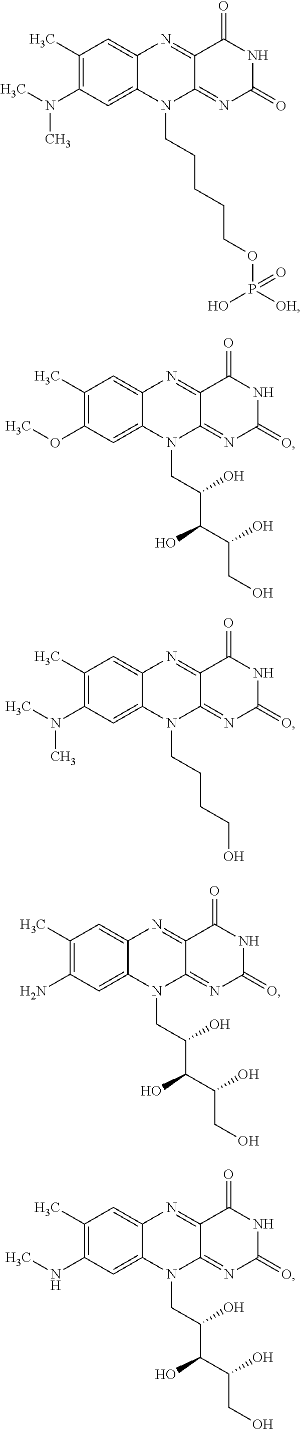

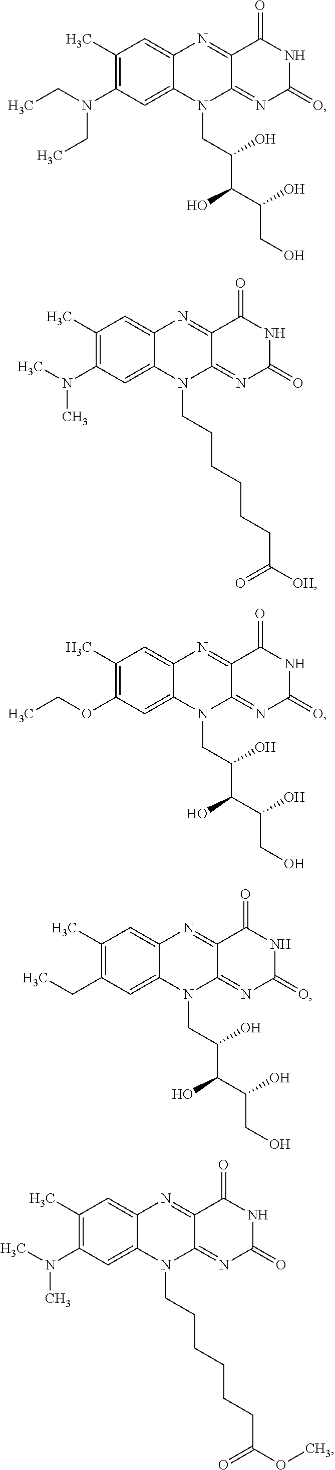

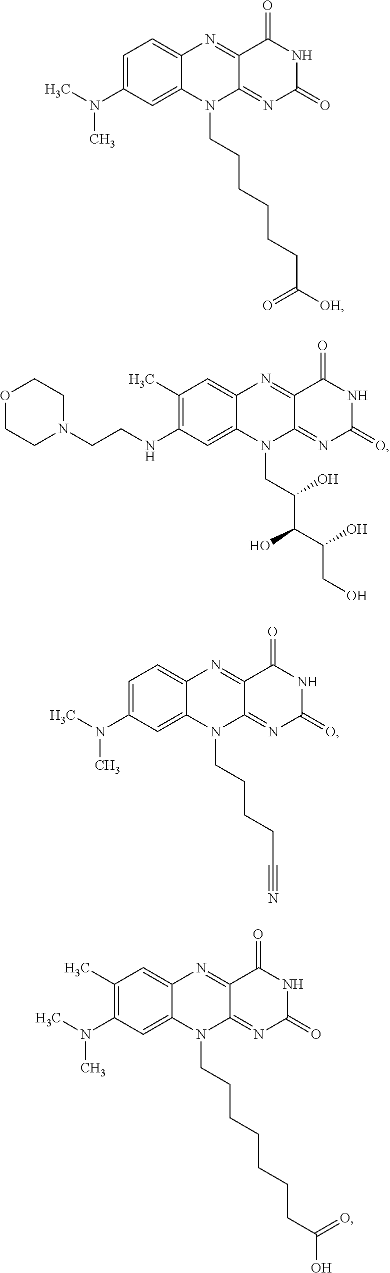

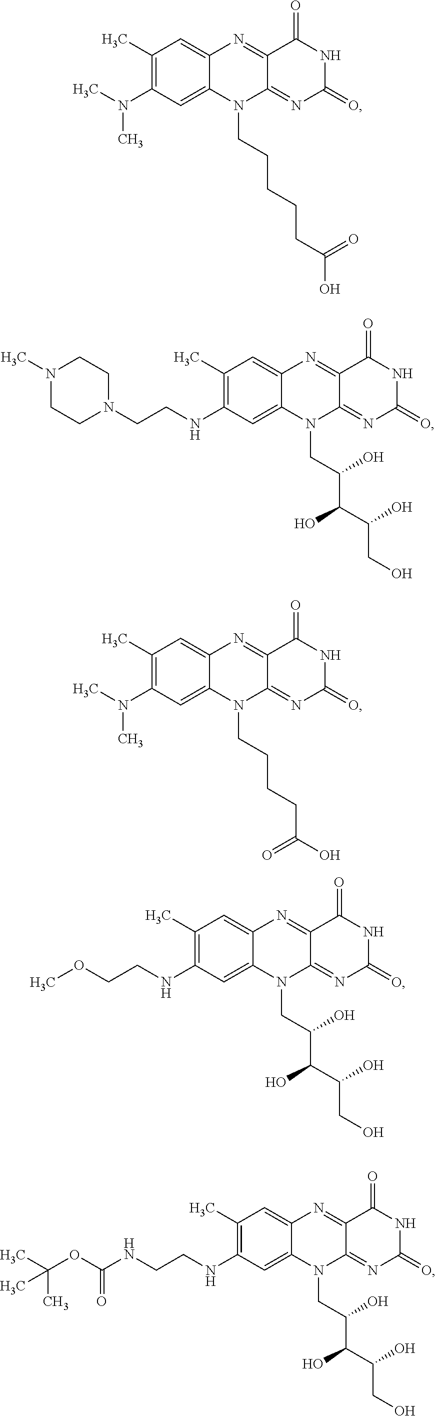

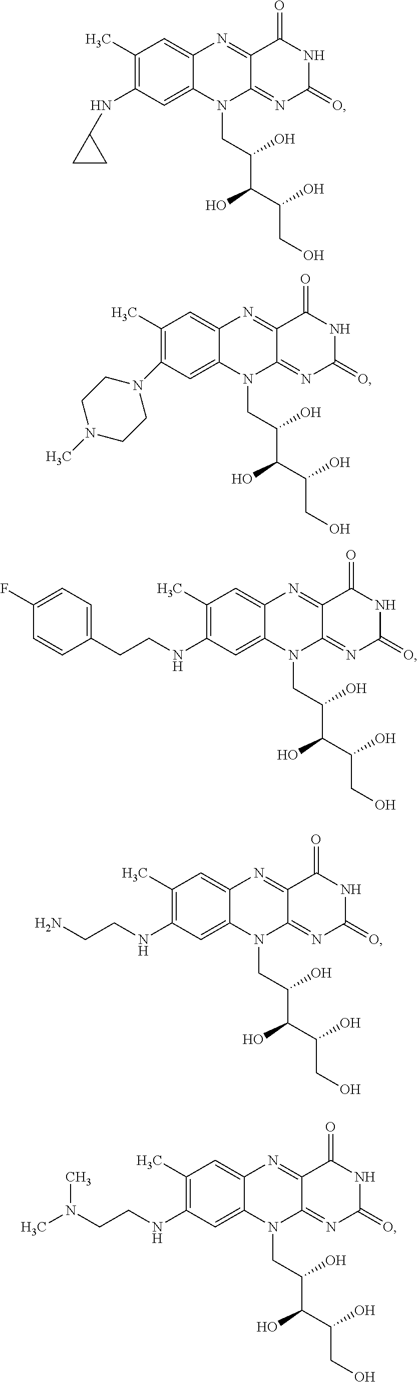

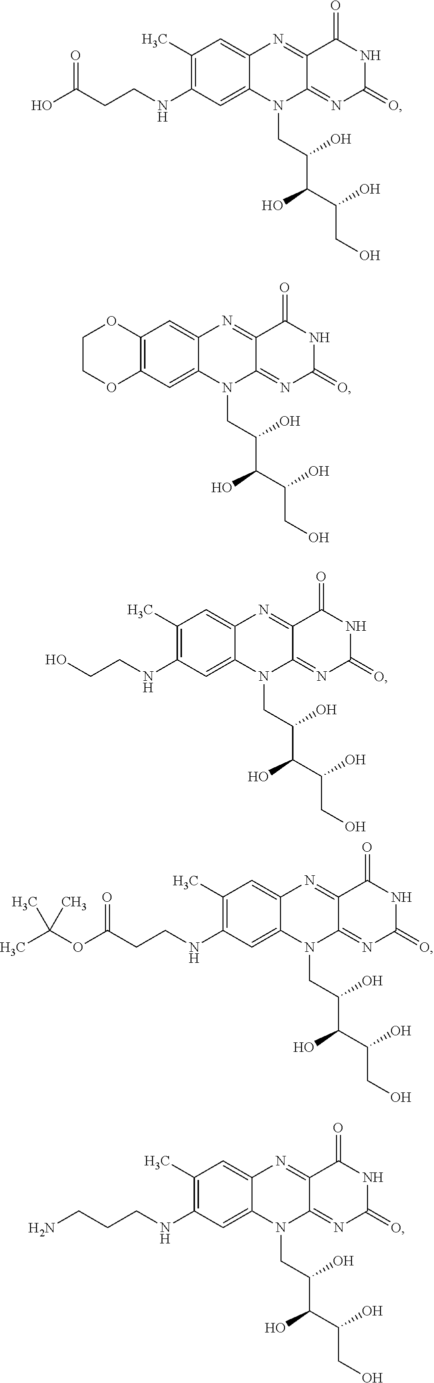

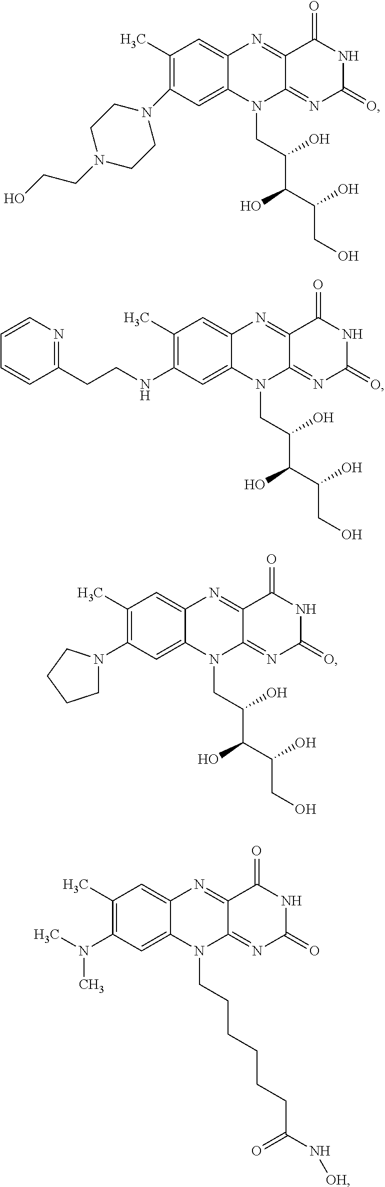

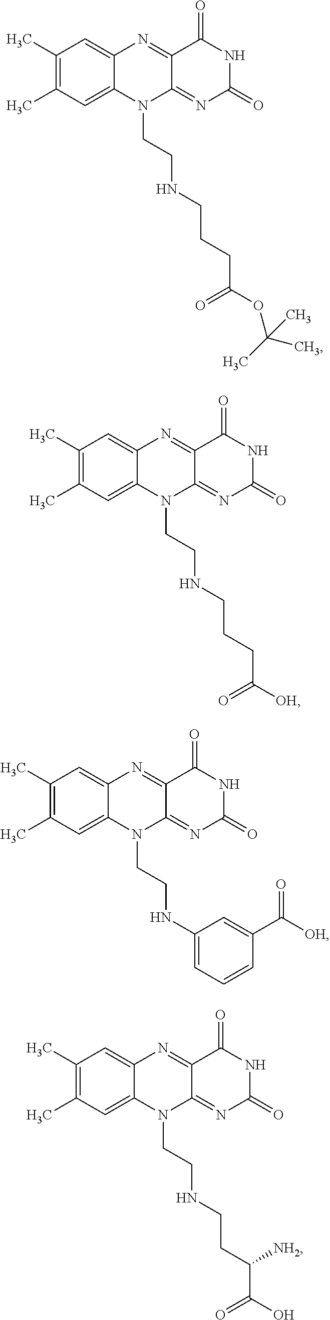

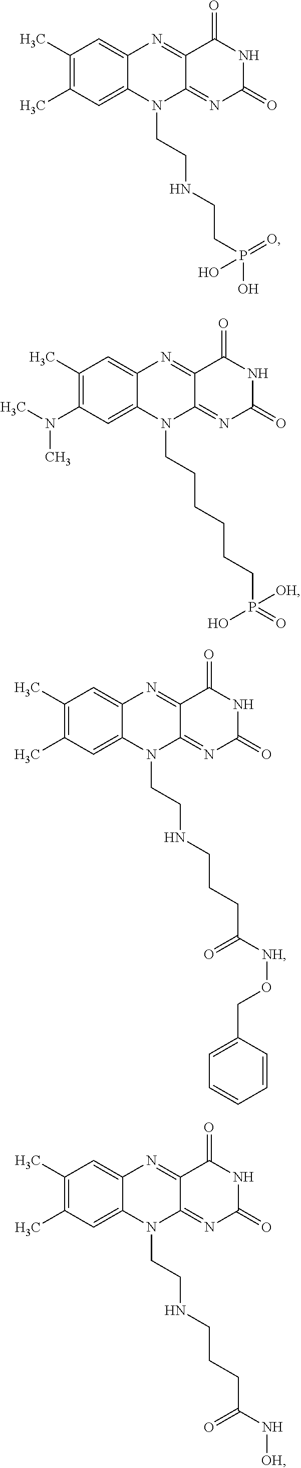

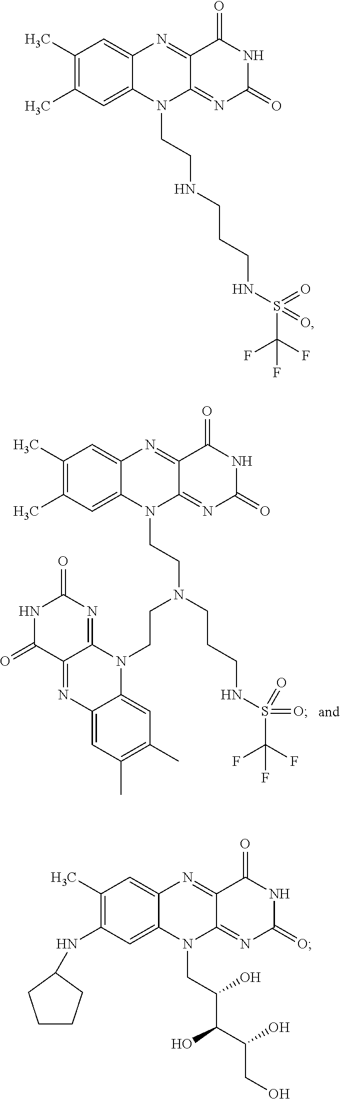

1.204 any of Formula I or 1.1-1.202 wherein a Compound of Formula I is selected from any of the following:

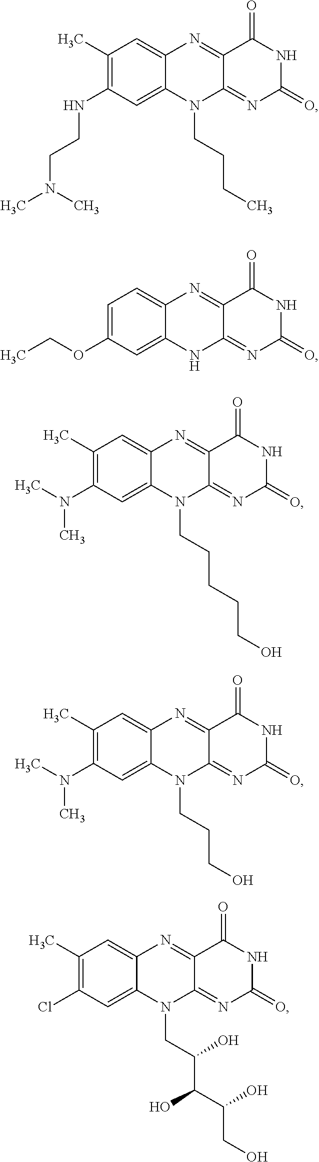

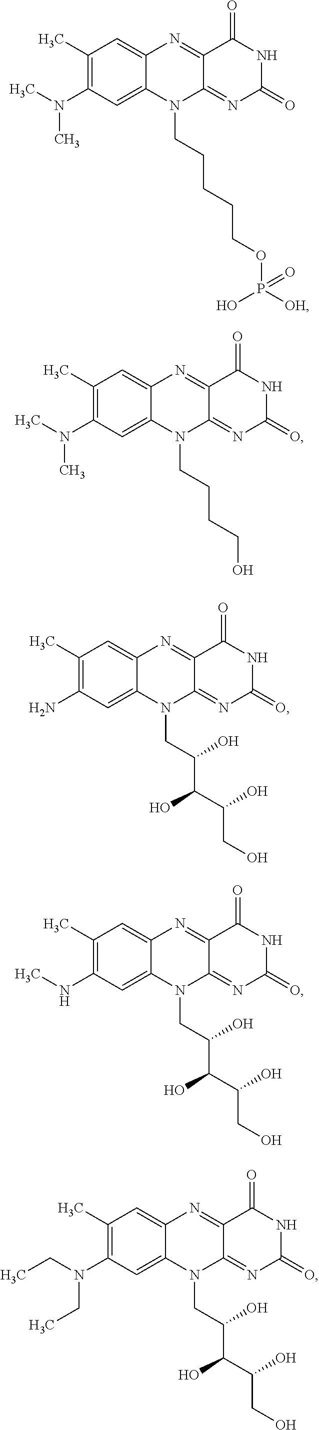

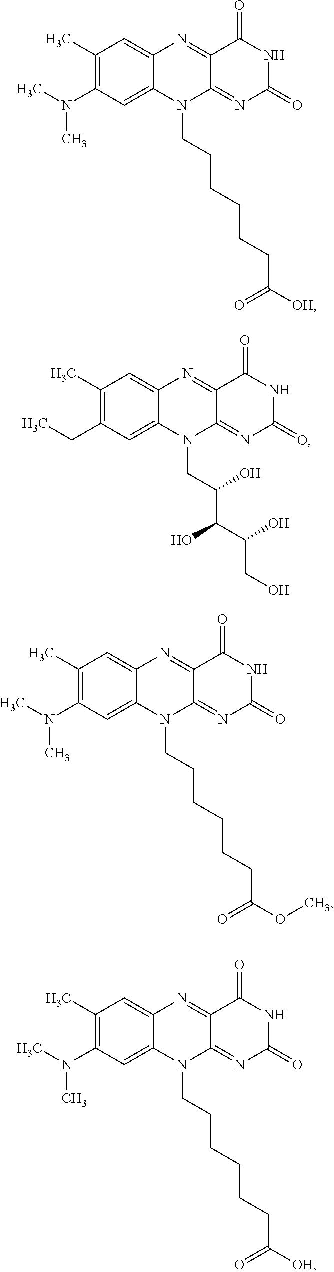

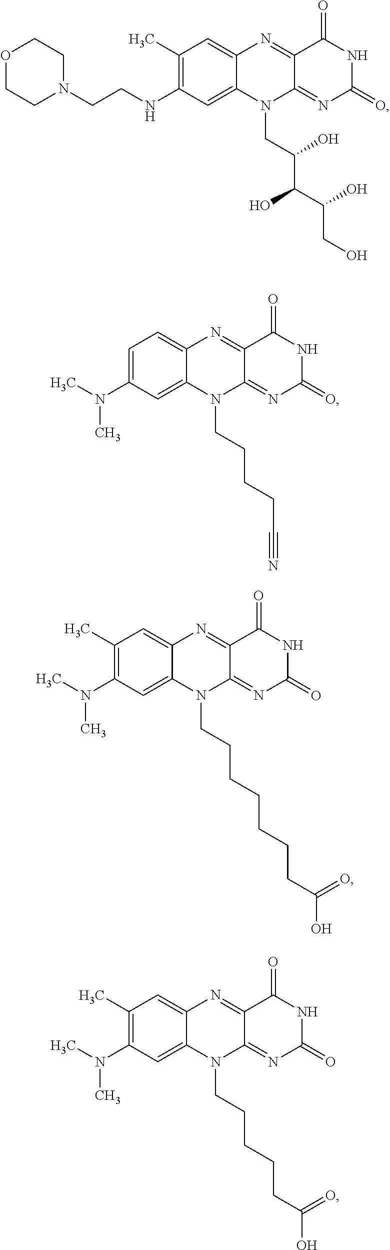

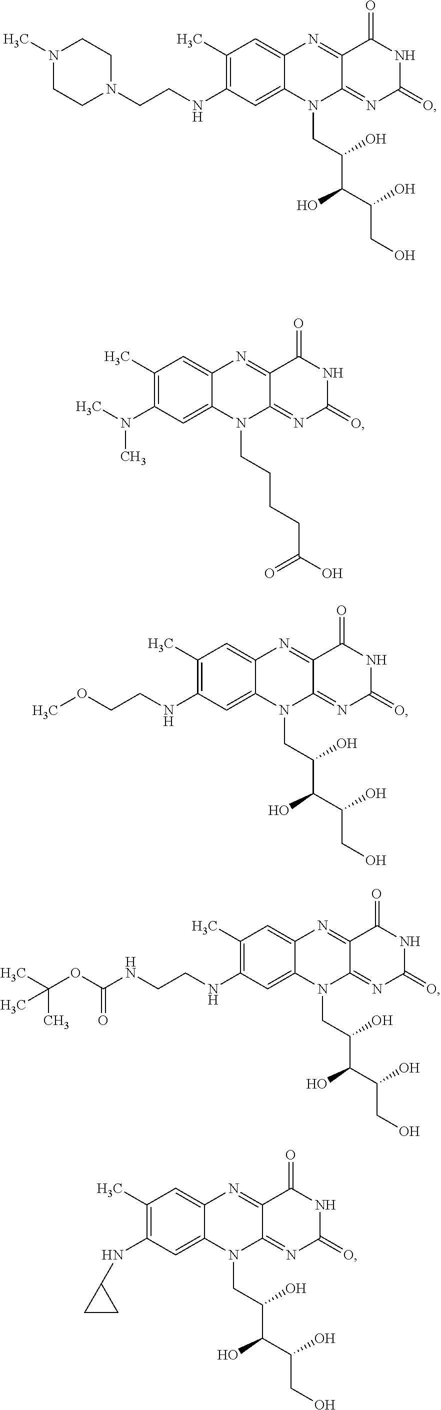

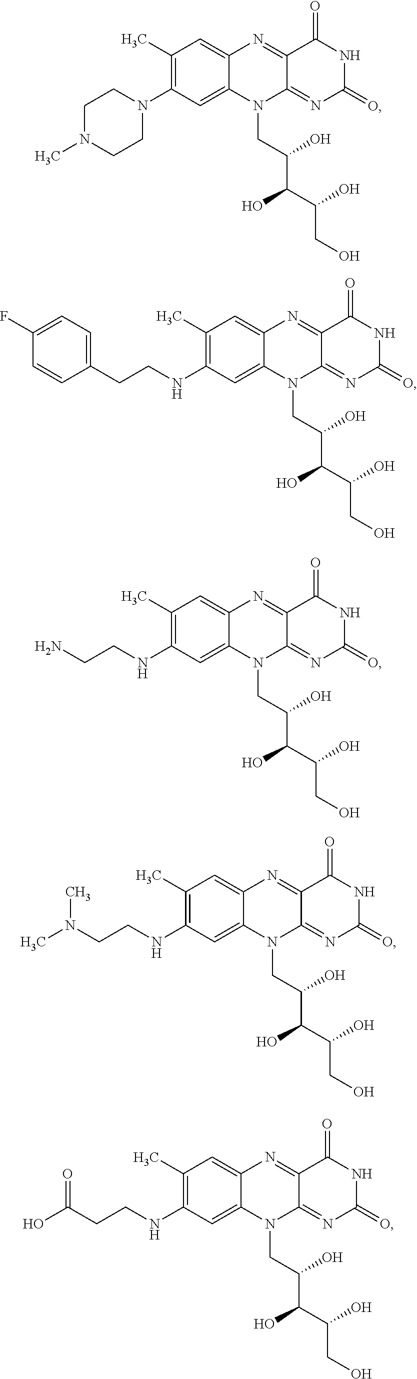

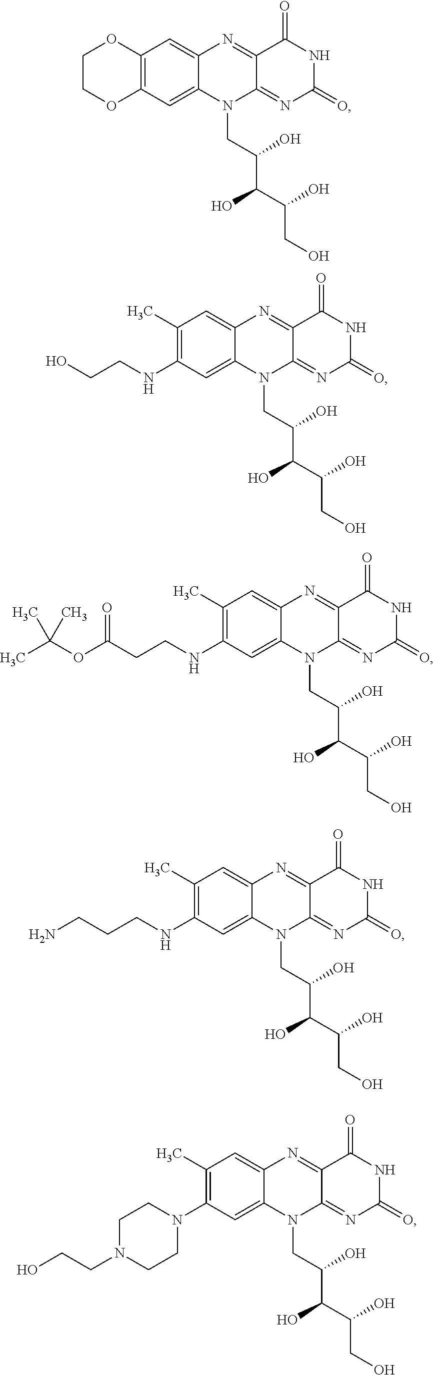

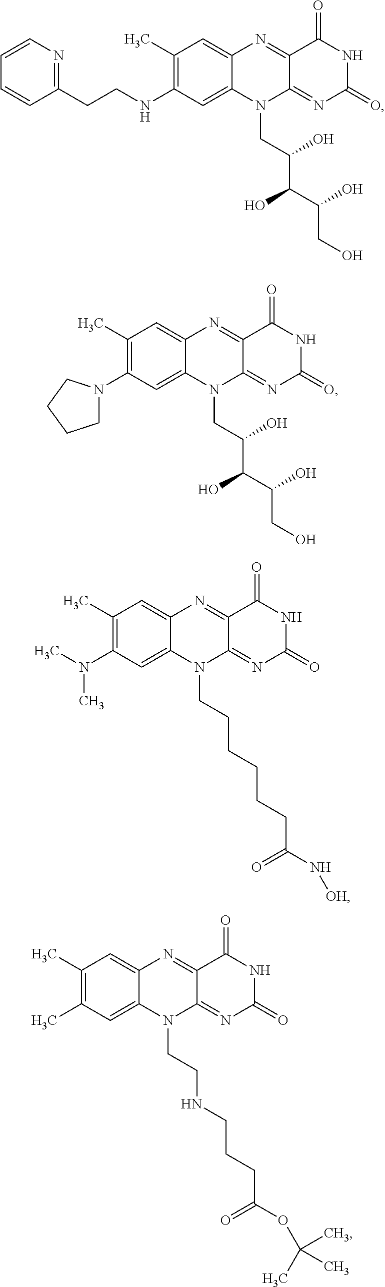

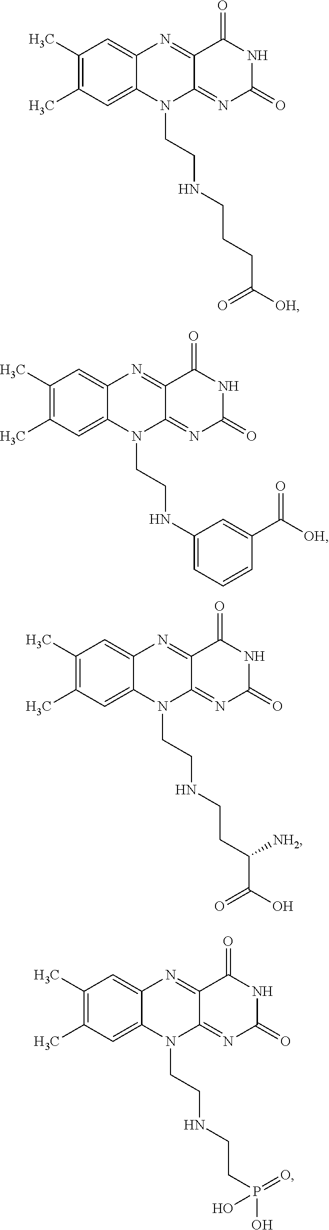

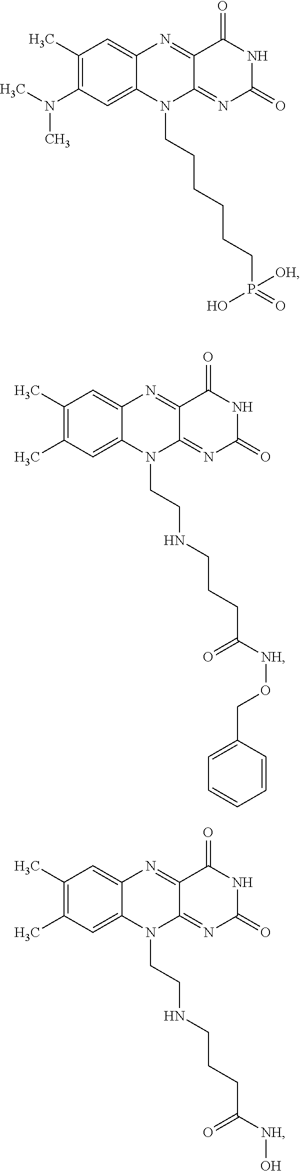

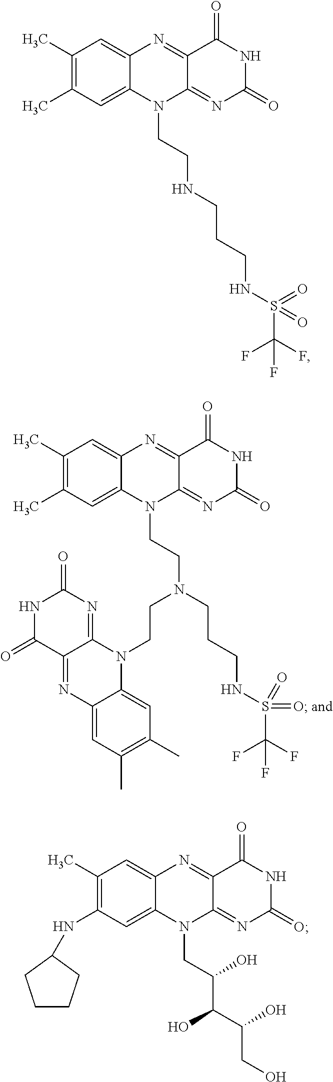

1.205 any of Formula I or 1.1-1.202 wherein a Compound of Formula I is selected from any of the following:

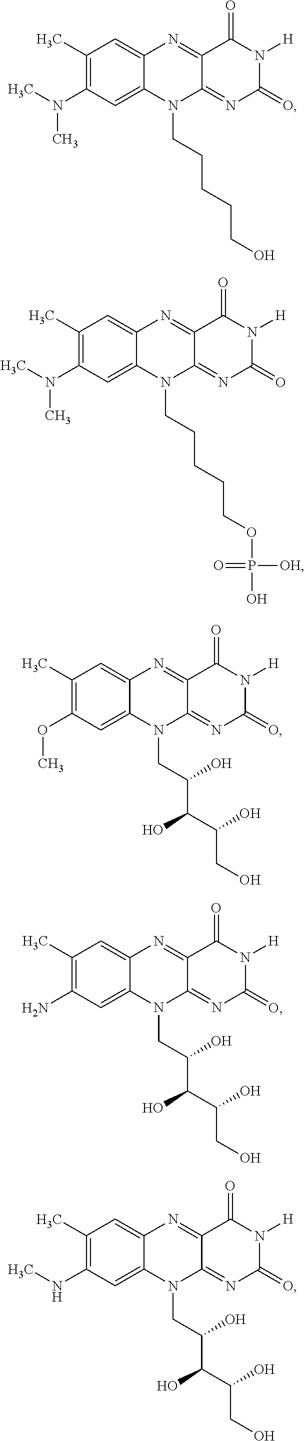

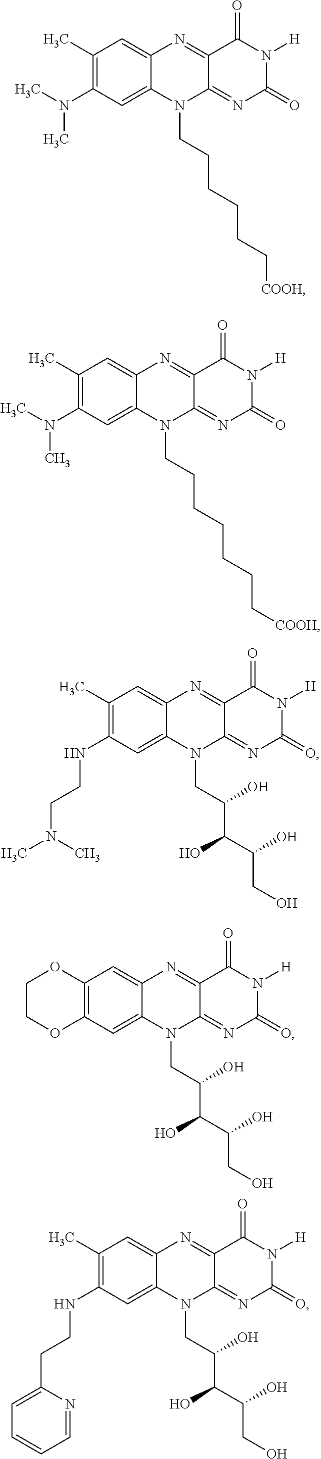

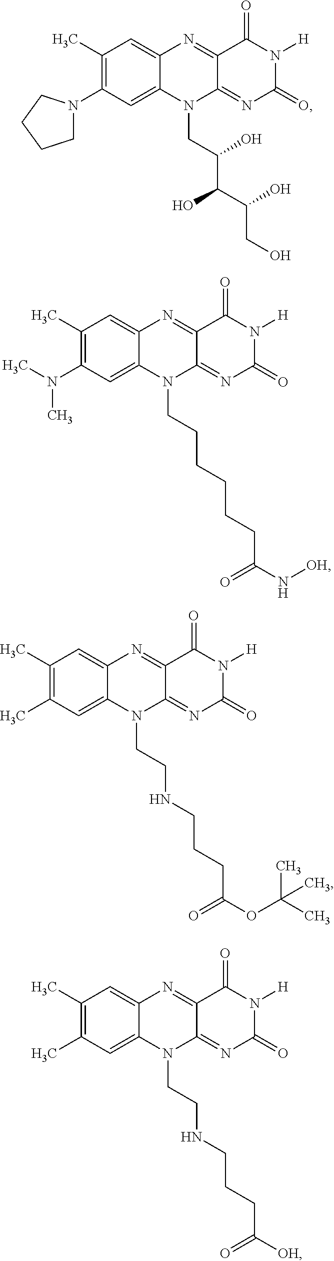

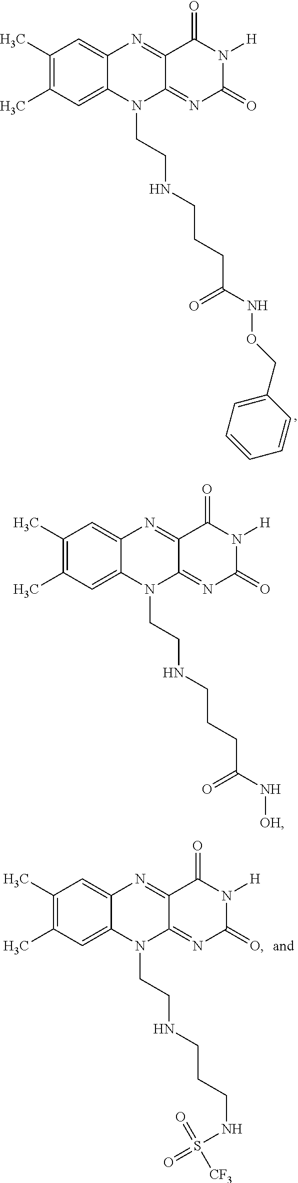

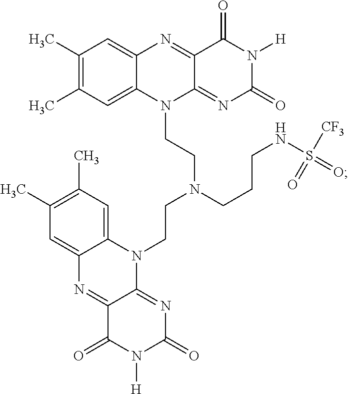

1.206 any of Formula I or 1.1-1.202 wherein a Compound of Formula I is selected from any of the following:

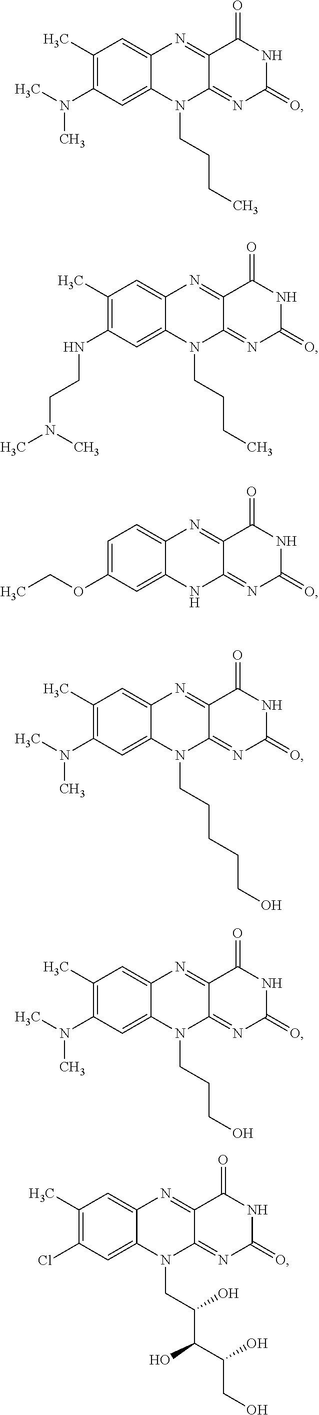

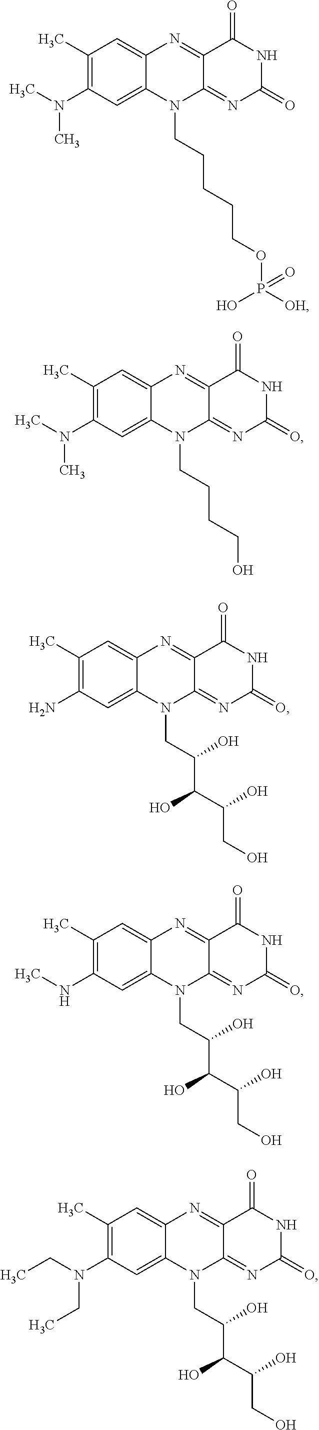

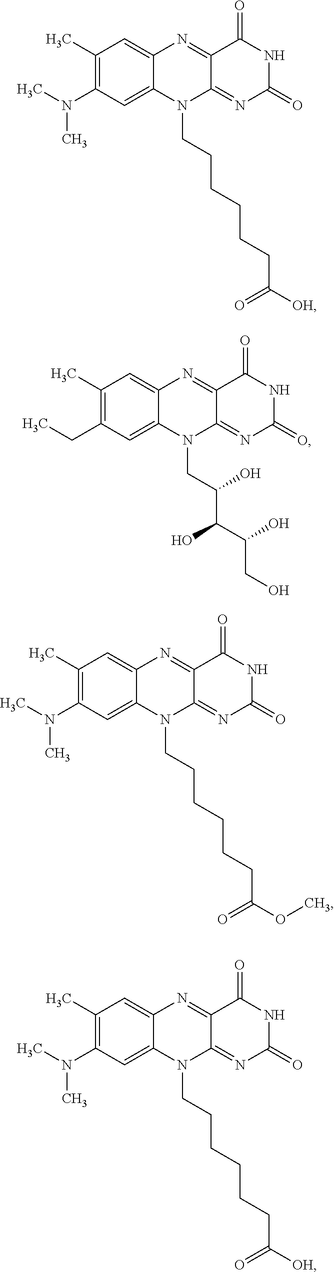

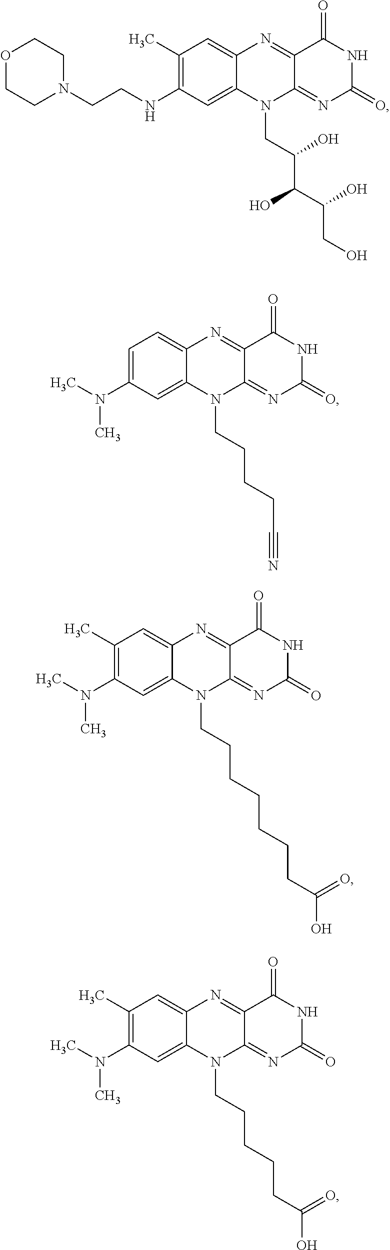

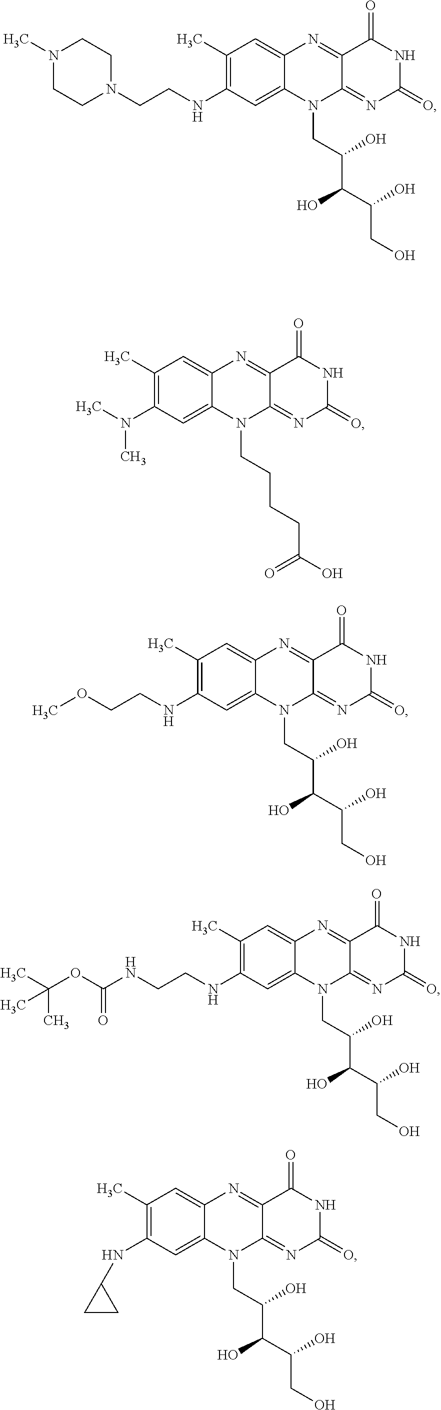

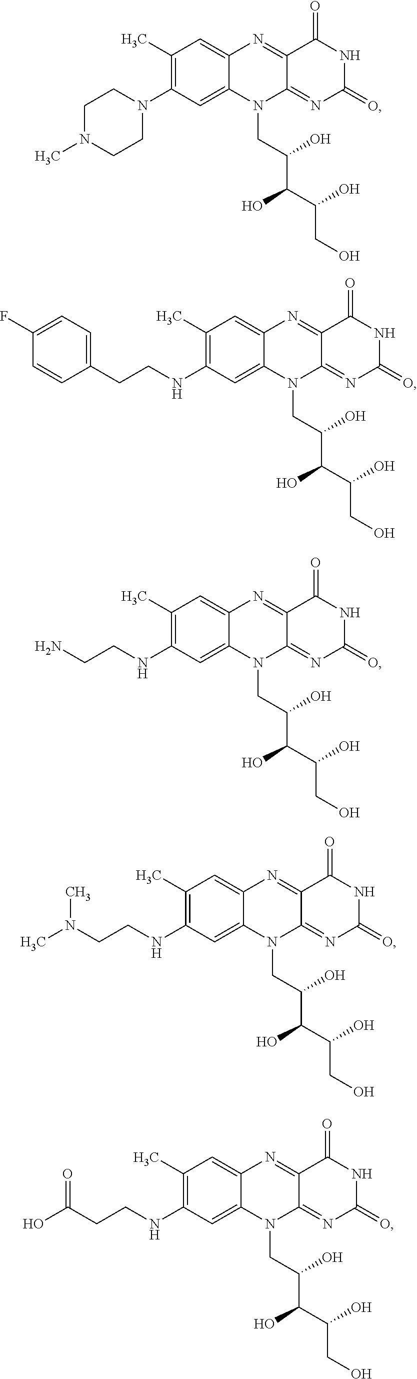

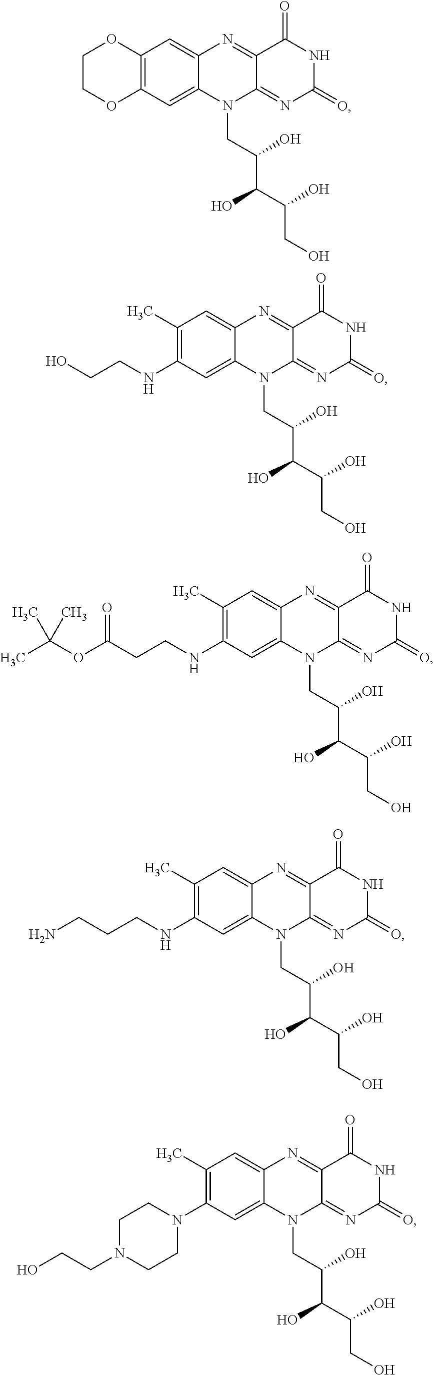

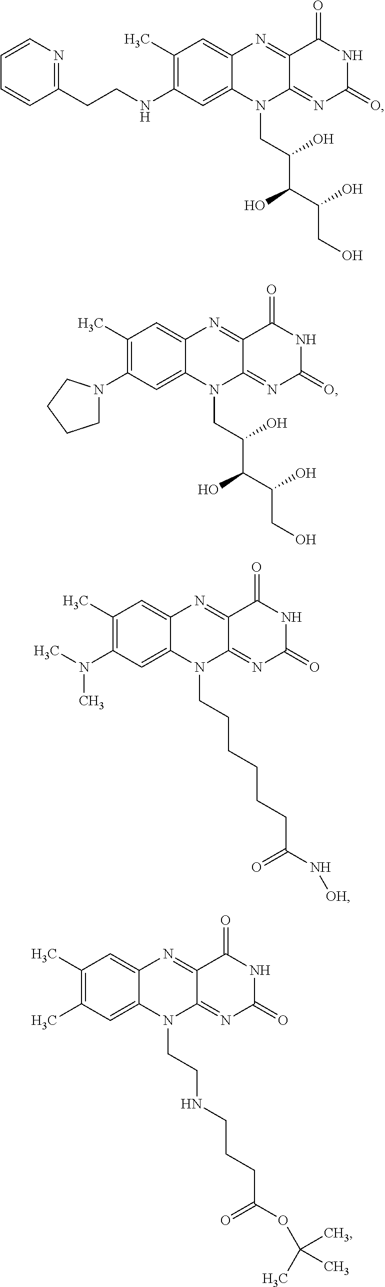

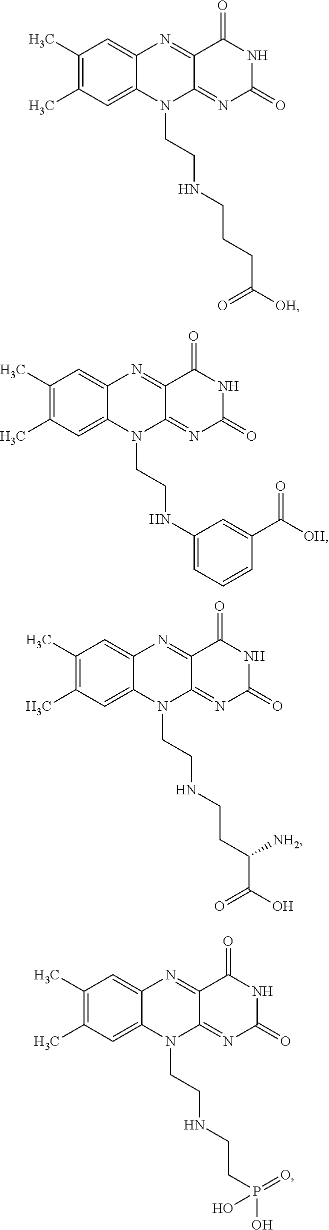

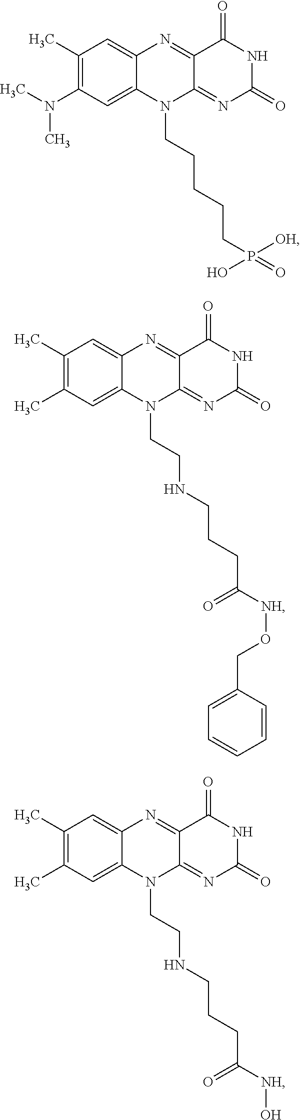

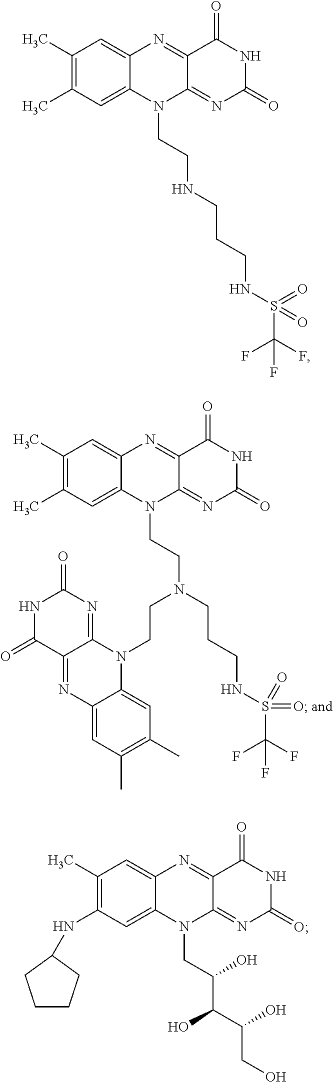

1.207 any of Formula I or 1.1-1.203 wherein a Compound of Formula I is selected from any of the following:

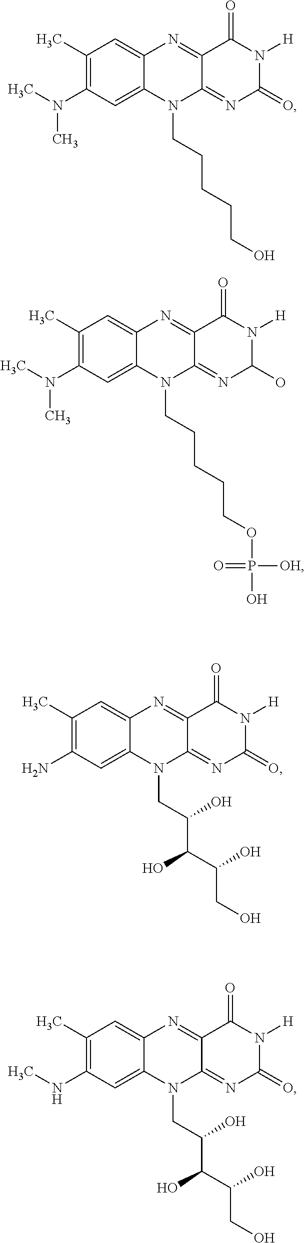

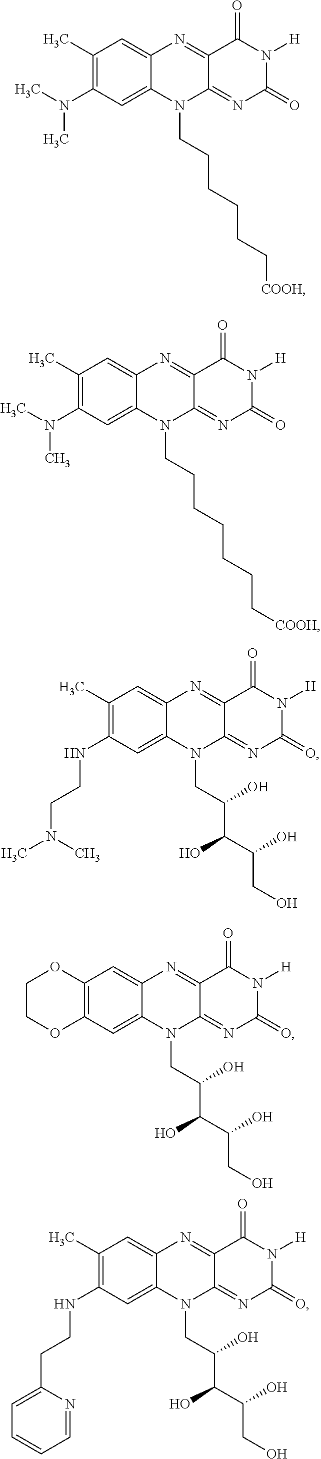

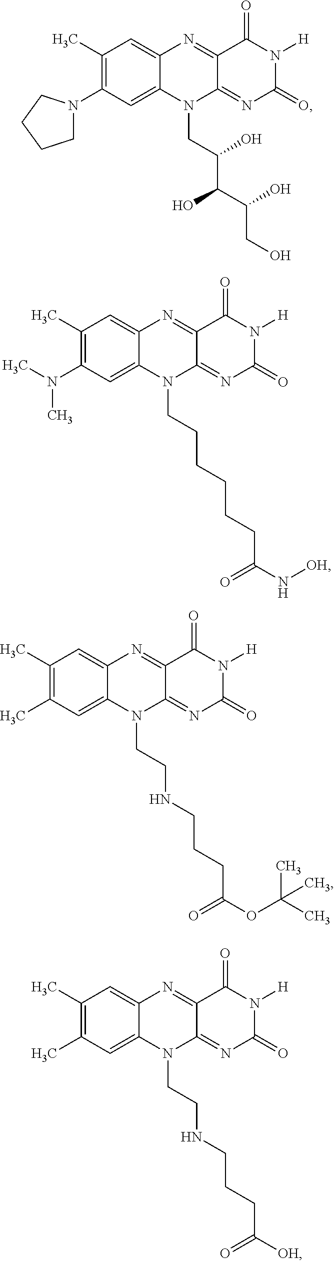

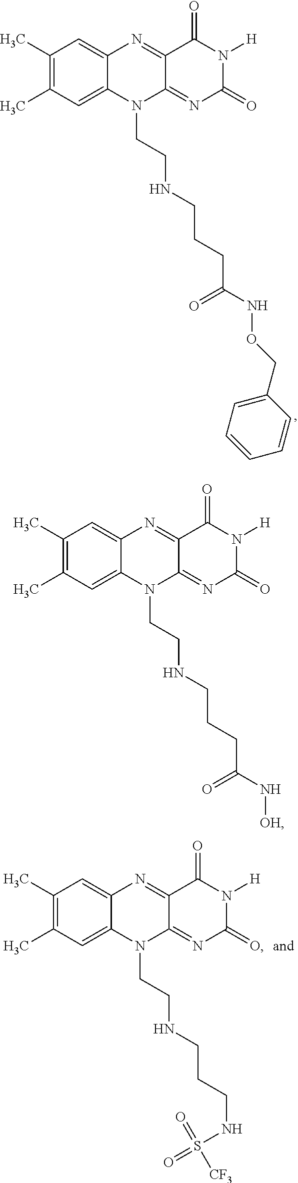

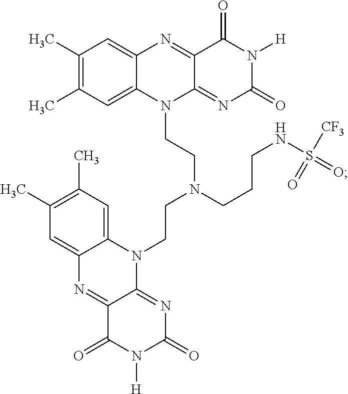

1.208 any of Formula I or 1.1-1.202 wherein a Compound of Formula I is selected from any of the following:

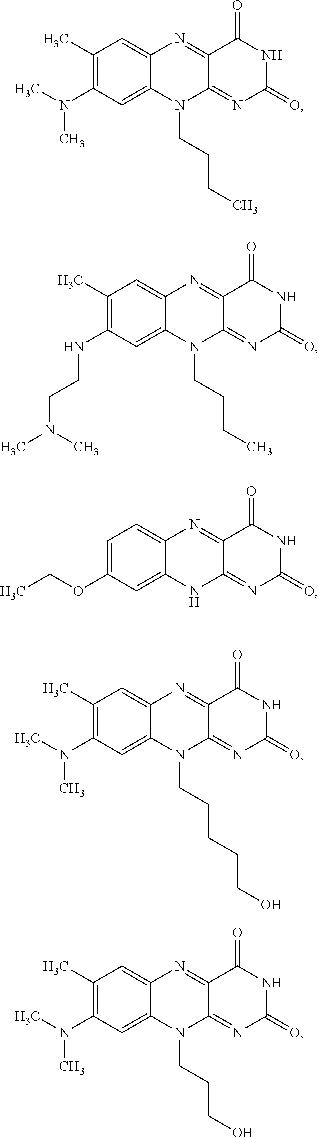

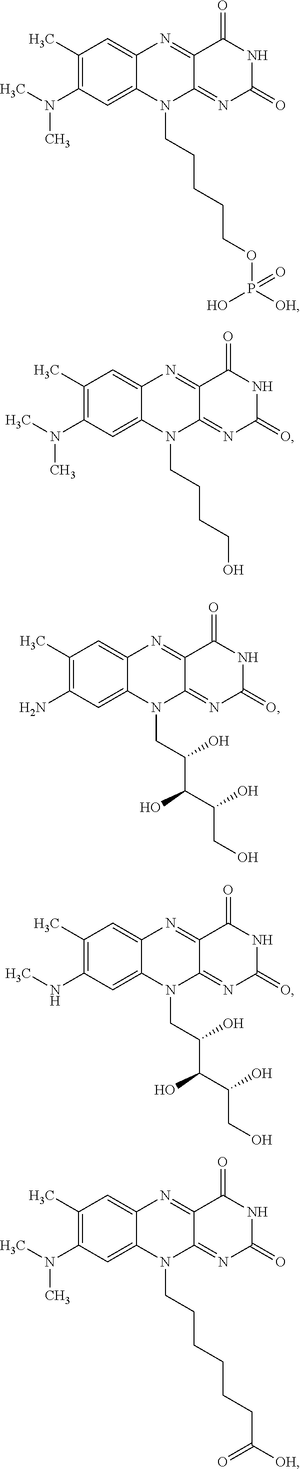

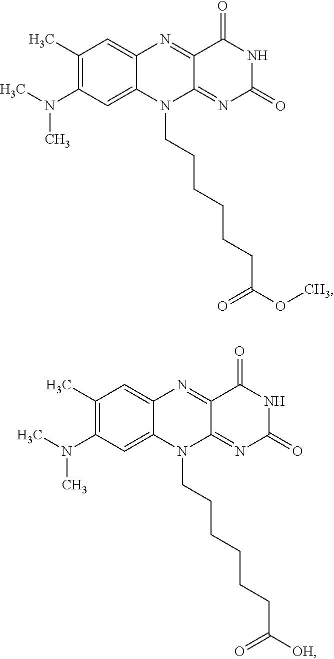

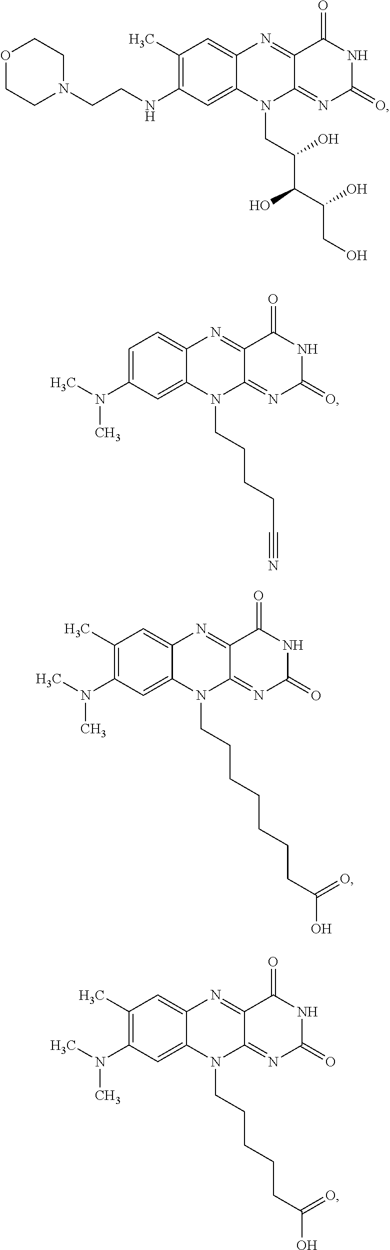

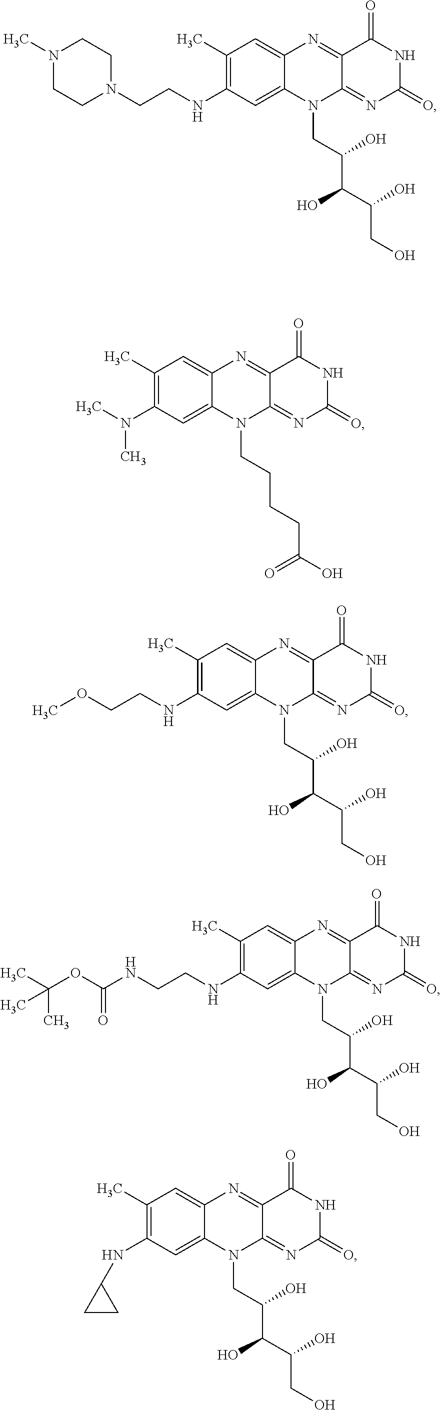

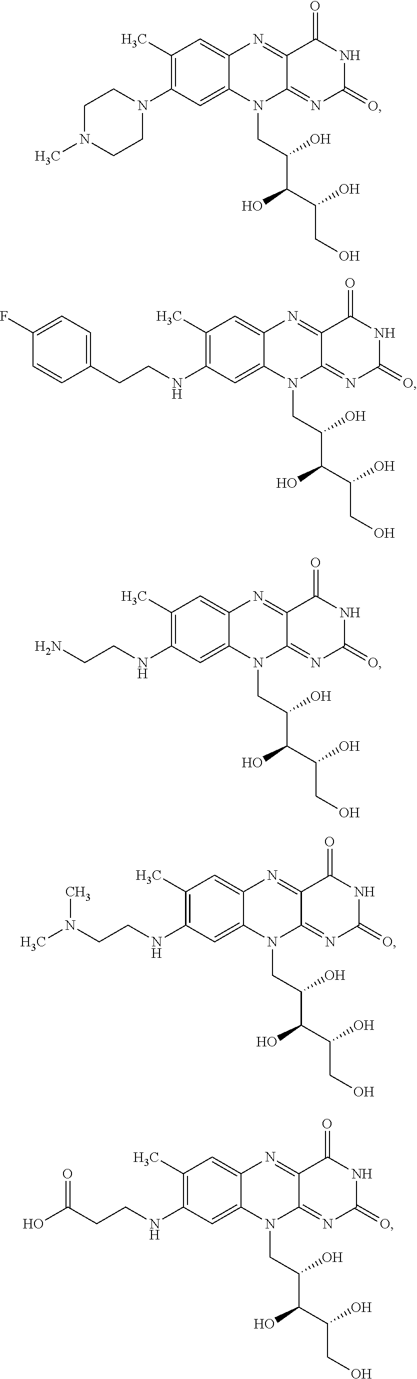

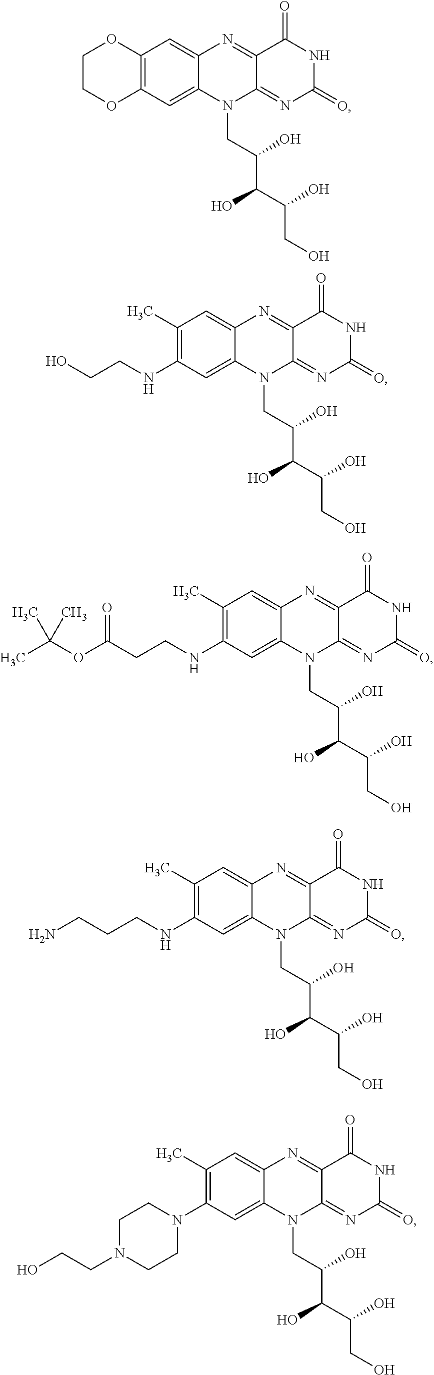

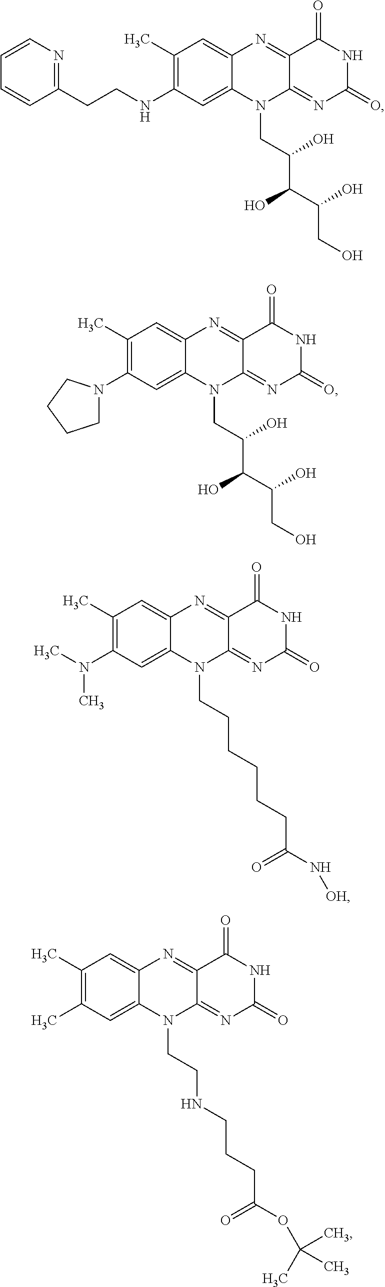

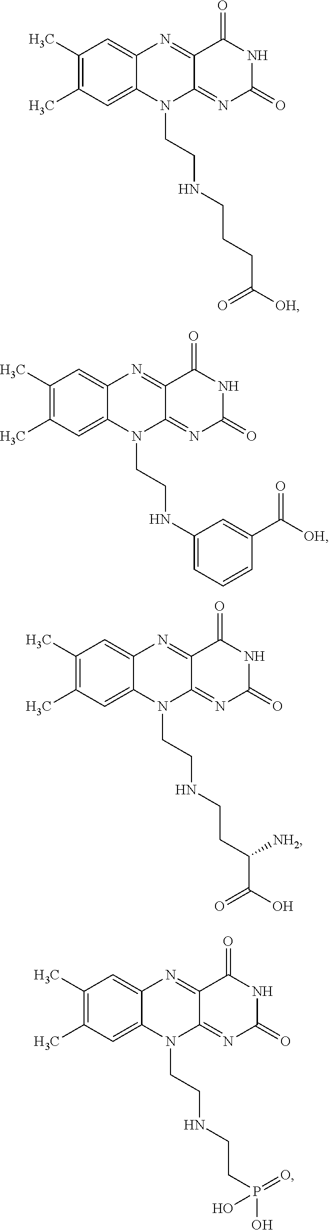

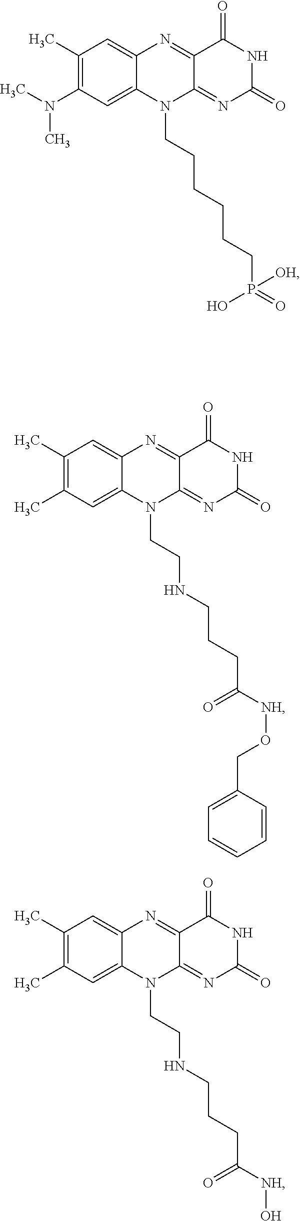

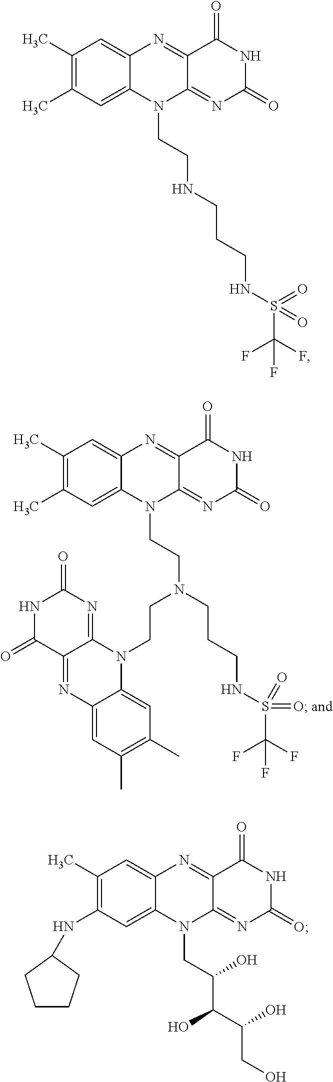

1.209 any of Formula I or 1.1-1.202 wherein a Compound of Formula I is selected from any of the following:

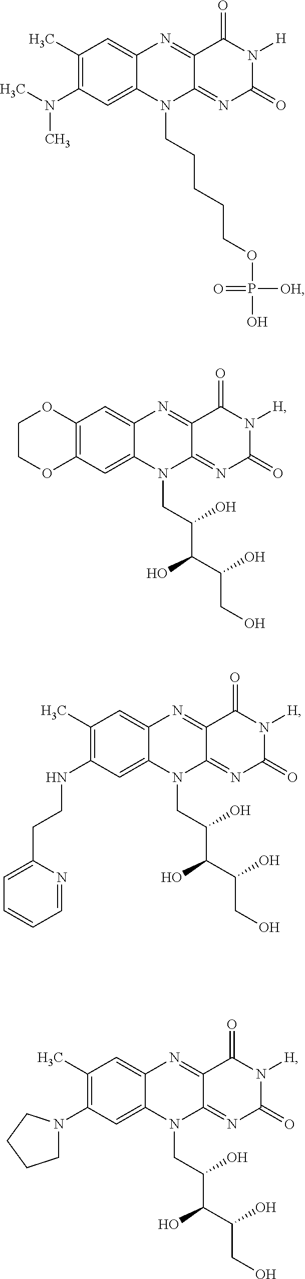

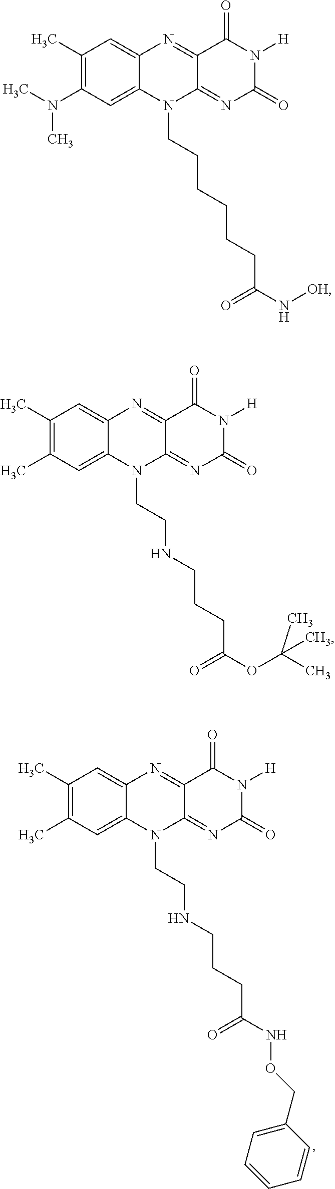

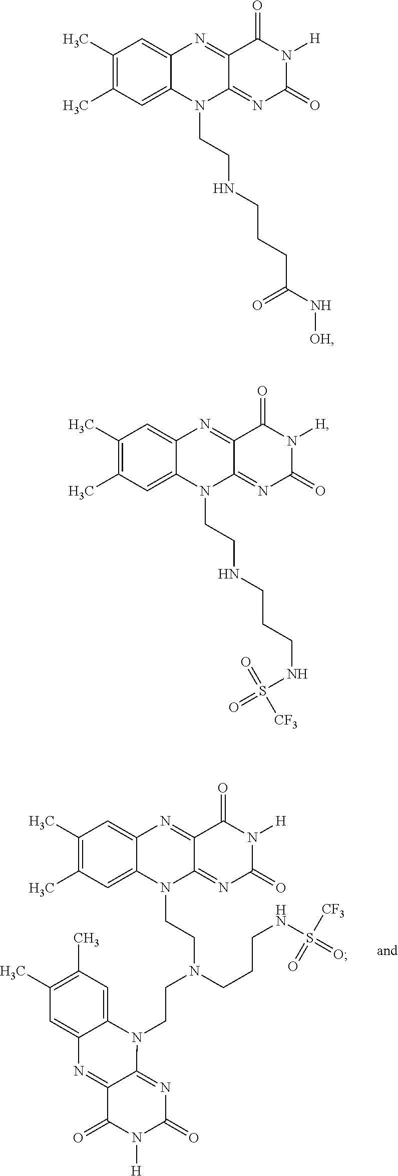

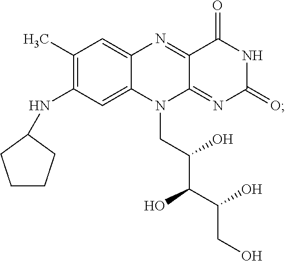

1.210 any of Formula I or 1.1-1.202 wherein the Compound of Formula I is selected from any of the following:

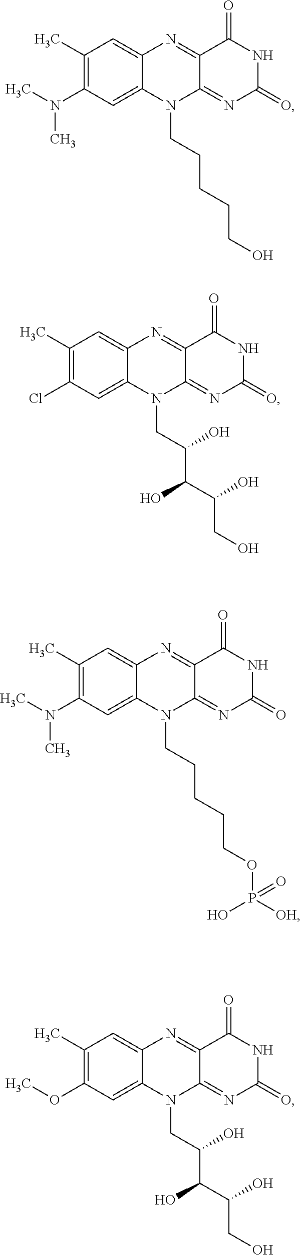

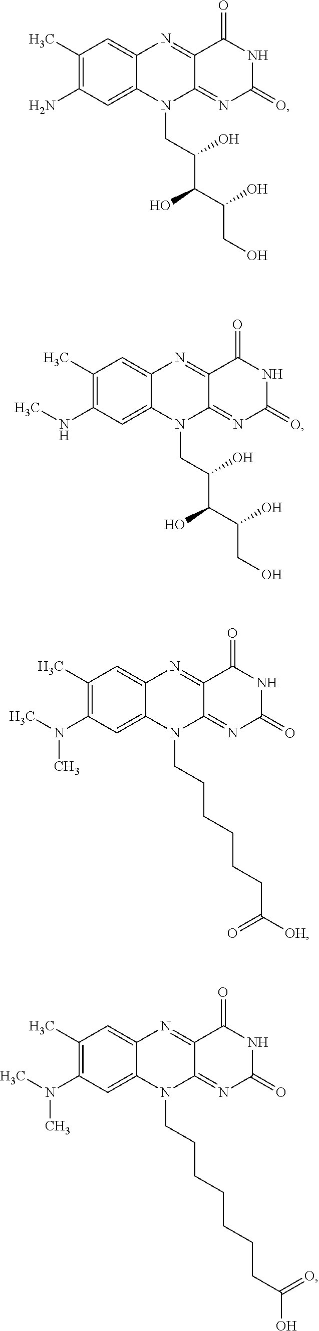

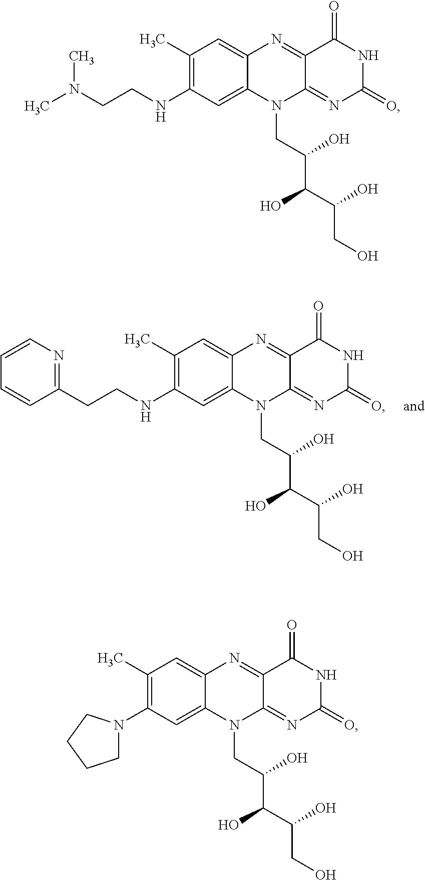

1.211 any of 1.1-1.202, wherein the Compound of Formula I is:

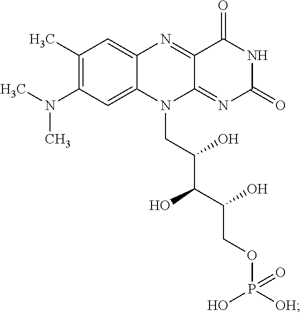

1.212 any of the preceding formulae wherein the Compound of Formula I binds to FMN riboswitch, e.g., with an IC₅₀ of less than or equal to 10 μM, preferably less than 1 μM, more preferably less than 100 nM, most preferably less than 10 nM in a binding assay, for example, as described in Example 1 and/or has a minimum inhibitory concentration (MIC) of less than 128 μg/mL, preferably less than 32 μg/mL, in an assay, for example, as described in Example 1A;
in free, salt or prodrug form.

In another embodiment, the invention relates to a compound of Formula I(i):

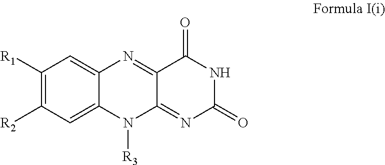

wherein
(i) R₁ is H, C₁₋₈ alkyl (e.g., methyl) or C₃₋₇ cycloalkyl;
(ii) R₂ is H, halo (e.g., chloro), C₁₋₈alkyl (e.g., methyl or ethyl), C₁₋₈alkoxy (e.g., methoxy or ethoxy), —N(R₄)(R₅), C₃₋₇cycloalkyl or C₄₋₇heterocycle (e.g., piperazinyl or pyrrolidinyl) wherein said heterocycle is optionally substituted with C₁₋₈alkyl (e.g., 4-methyl-piperazin-1-yl) or hydroxyC₁₋₈alkyl (e.g., 4-hydroxyethyl-piperazin-1-yl); or
(iii) R₁ and R₂ are connected so as to form a cyclic ring structure optionally containing one or more heteroatoms selected from N, O and S (e.g., —OCH₂CH₂O—);
(iv) R₃ is H or C₁₋₈ alkyl (e.g., methyl, ethyl, n-butyl, n-pentyl, n-propyl, n-hexyl or n-heptyl), wherein the alkyl group is optionally substituted with one or more groups selected from —OP(O)(OR₉)(OR₁₇), —OP(O)(OR₉)NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇);
(v) R₄ and R₅ are independently selected from H, C₃₋₇ cycloalkyl (e.g., cyclopropyl or cyclopentyl), C₄₋₇heterocycle (e.g., piperazinyl), and C₁₋₈alkyl (e.g., methyl, ethyl or 2,2-dimethylpropyl) wherein said alkyl is optionally substituted with one or more groups selected from —OR₁₁, —C(O)OR₉, —N(R₆)(R₇) (e.g., amino or dimethylamino), C₁₋₈alkoxyl (e.g., methoxy), C₆₋₁₀aryl (e.g., phenyl), C₅₋₁₀ heteroaryl (e.g., pyridinyl) wherein said aryl or heteroaryl are optionally substituted with halo (e.g., 4-fluorophenyl), and C₄₋₇heterocycle wherein said heterocycle is optionally substituted with C₁₋₈alkyl (e.g., morpholin-4-yl or 4-methylpiperazin-1-yl);
(vi) R₆ and R₇ are independently selected from H, C₁₋₈alkyl (e.g., methyl, ethyl, n-propyl, n-butyl), —C₁₋₈alkyl-OR₁₁, —C(O)OR₉, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-C₁₋₈alkyl and aryl wherein said aryl and alkyl are optionally substituted with —COOR₉;
(vii) R₈ is H, C₁₋₈alkyl (e.g., methyl, ethyl or t-butyl), —OR₁₁ or —OBn;
(viii) R₉ and R₁₇ are independently selected from H, C₁₋₈alkyl (e.g., methyl, ethyl, n-butyl or t-butyl), —C₁₋₄alkyl-OC(O)R₁₂, phenyl and Bn wherein said phenyl and Bn are optionally substituted with one or more halo or C₁₋₄alkoxy (e.g., 3-chloro-phenylmethyl, 3-fluoro-phenylmethyl, 4-methoxy-3-fluorophenylmethyl);
(ix) R₁₀ is H, C₁₋₈alkyl (e.g., methyl or ethyl), —C₁₋₈allyl-OR₁₁, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl, or aryl wherein said aryl and alkyl are optionally substituted with —COOR₉, or —C₁₋₄alkyl-OC(O)R₁₂;
(x) R₁₁ is H, or —C₁₋₄alkyl-OC(O)R₁₂ (e.g., —CH₂—OC(O)R₁₂);
(xi) R₁₂ is C₁₋₈alkyl (e.g., methyl, ethyl, t-Butyl) or —OC₁₋₈alkyl (e.g., methoxy, ethoxy, t-butoxy);
(xii) R₁₃, R₁₄, R₁₅ and R₁₆ are independently selected from H, C₁₋₈alkyl, and —C₁₋₈alkyl-COOR₁₈ (e.g., —CH(methyl)-COOR₁₈, —CH(isopropyl)-COOR₁₈, —CH(isobutyl)-COOR₁₈, —CH(sec-butyl)-COOR₁₈), wherein the alkyl group of C₁₋₈alkyl-COOR₁₈ is optionally substituted with hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH);
(xiii) R₁₈ is H or C₁₋₈alkyl (e.g., ethyl);
in free, salt or prodrug form.

In still another embodiment, the invention relates to a compound of Formula I(ii):

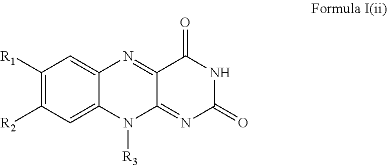

wherein
(i) R₁ is H, C₁₋₈ alkyl (e.g., methyl);
(ii) R₂ is H, halo (e.g., chloro), C₁₋₈alkyl (e.g., methyl or ethyl), C₁₋₈alkoxy (e.g., methoxy or ethoxy), —N(R₄)(R₅);
(iii) R₃ is C₁₋₈ alkyl (e.g., methyl, ethyl, n-propyl, n-butyl, n-pentyl, n-hexyl or n-heptyl), wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇);
(iv) R₄ and R₅ are independently selected from H, C₃₋₇ cycloalkyl (e.g., cyclopropyl or cyclopentyl), and C₁₋₈alkyl (e.g., methyl, ethyl or 2,2-dimethylpropyl) wherein said alkyl is optionally substituted with one or more groups selected from —OR₁₁;
(v) R₆ and R₇ are independently selected from H, C₁₋₈alkyl (e.g., methyl, ethyl, n-propyl, n-butyl), —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), 7,8-dimethyl-isoalloxazin-10-yl-C₁₋₈alkyl and aryl wherein said aryl and alkyl are optionally substituted with —COOR₉;
(vi) R₈ is H, C₁₋₈alkyl (e.g., methyl, ethyl or t-butyl);
(vii) R₉ and R₁₇ are independently selected from H, C₁₋₈alkyl (e.g., methyl, ethyl, n-butyl or t-butyl), —C₁₋₄alkyl-OC(O)R₁₂, phenyl and Bn wherein said phenyl and Bn are optionally substituted with one or more halo or C₁₋₄alkoxy (e.g., 3-chloro-phenylmethyl, 3-fluoro-phenylmethyl, 4-methoxy-3-fluorophenylmethyl);
(viii) R₁₃ is —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-C₁₋₈alkyl, or aryl wherein said aryl and alkyl are optionally substituted with —COOR₉, or —C₁₋₄alkyl-OC(O)R₁₂;
(ix) R₁₁ is H, or —C₁₋₄alkyl-OC(O)R₁₂ (e.g., —CH₂—OC(O)R₁₂);
(x) R₁₂ is C₁₋₈allyl (e.g., methyl, ethyl, t-Butyl) or —OC₁₋₈alkyl (e.g., methoxy, ethoxy, t-butoxy);
(xi) R₁₃, R₁₄, R₁₅ and R₁₆ are independently selected from H, C₁₋₈alkyl, and —C₁₋₈alkyl-COOR₁₈ (e.g., —CH(methyl)-COOR₁₈, —CH(isopropyl)-COOR₁₈, —CH(isobutyl)-COOR₁₈, —CH(sec-butyl)-COOR₁₈), wherein the alkyl group of C₁₋₈alkyl-COOR₁₈ is optionally substituted with hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH); and
(xii) R₁₈ is H or C₁₋₈alkyl (e.g., ethyl);
in free, salt or prodrug form.

The invention further relates to a compound of Formula I(iii) as follows:

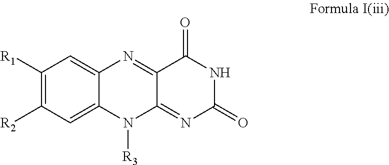

wherein
(i) R₁ is H, C₁₋₈ alkyl (e.g., methyl);
(ii) R₂ is H, halo (e.g., chloro), C₁₋₈alkyl (e.g., methyl or ethyl), C₁₋₈alkoxy (e.g., methoxy), —N(R₄)(R₅);
(iii) R₃ is C₁₋₈ alkyl (e.g., methyl, ethyl, n-butyl, n-pentyl, n-propyl, n-hexyl or n-heptyl), wherein the alkyl group is optionally substituted with one or more groups selected from —P(O)(OR₉)(OR₁₇), —P(O)(OR₉)(NR₁₃R₁₄), —P(O)(NR₁₃R₁₄)(NR₁₅R₁₆), —C(O)OR₉, —OR₁₀, —C(O)N(R₆)(R₇), and —N(R₆)(R₇);
(iv) R₄ and R₅ are independently selected from H, C₃₋₇ cycloalkyl (e.g., cyclopropyl or cyclopentyl), and C₁₋₈alkyl (e.g., methyl, ethyl or 2,2-dimethylpropyl) wherein said alkyl is optionally substituted with one or more groups selected from —OH;
(v) R₆ and R₇ are independently selected from H, —C₁₋₈alkyl (e.g., methyl), —C₁₋₈alkyl-C(O)OR₉, —C₁₋₈allyl(amine)-C(O)OR₉ (e.g., —CH₂CH₂CH(NH₂)COOH), —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl, wherein said aryl and alkyl are optionally substituted with —COOR₉;
(vi) R₉ and R₁₇ are independently selected from H, C₁₋₈alkyl (e.g., methyl, ethyl, n-butyl or t-butyl), —C₁₋₄alkyl-OC(O)R₁₂;
(vii) R₁₀ is H, —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆);
(viii) R₁₂ is C₁₋₈alkyl (e.g., methyl, ethyl, t-Butyl);
(ix) R₁₃, R₁₄, R₁₅ and R₁₆ are independently selected from H, C₁₋₈alkyl, and —C₁₋₈alkyl-COOR₁₈ (e.g., —CH(methyl)-COOR₁₈, —CH(isopropyl)-COOR₁₈, —CH(isobutyl)-COOR₁₈, —CH(sec-butyl)-COOR₁₈);
(x) R₁₈ is H or C₁₋₈alkyl (e.g., ethyl); in free, salt or prodrug form.

In still another embodiment, the invention relates to a compound of Formula I(iv):

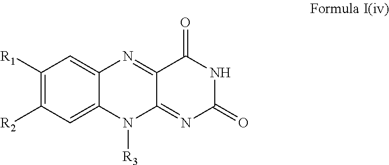

wherein
(i) R₁ is H, C₁₋₈ alkyl (e.g., methyl);
(ii) R₂ is H, halo (e.g., chloro), C₁₋₈alkyl (e.g., methyl or ethyl), C₁₋₈alkoxy (e.g., methoxy or ethoxy), —N(R₄)(R₅);
(iii) R₃ is C₁₋₈alkyl-N(R₆)(R₇), C₁₋₈alkyl-C(O)N(R₆)(R₇), C₁₋₈alkyl-P(O)(OR₉)(OR₁₇), C₁₋₈alkyl-P(O)(OR₉)(NR₁₃R₁₄), C₁₋₈alkyl-P(O)(NR₁₃R₁₄)(NR₁₅R₁₆), C₁₋₈alkyl-C(O)OR₉, C₁₋₈alkyl-OR₁₀;
(iv) R₄ and R₅ are independently selected from H, C₃₋₇ cycloalkyl (e.g., cyclopropyl or cyclopentyl), and C₁₋₈alkyl (e.g., methyl, ethyl or 2,2-dimethylpropyl) wherein said alkyl is optionally substituted with one or more groups selected from —OR₁₁;
(v) R₆ and R₇ are independently selected from H, C₁₋₈alkyl (e.g., methyl, ethyl, n-propyl, n-butyl), —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl and aryl wherein said aryl and alkyl are optionally substituted with —COOR₉;
(vi) R₉ and R₁₇ are independently selected from H, C₁₋₈alkyl (e.g., methyl, ethyl, n-butyl or t-butyl), —C₁₋₄alkyl-OC(O)R₁₂;
(vii) R₁₀ is H, —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O) (OR₉)(NR₁₃R₁₄), —C₁₋₈allyl-P(O)(NR₁₃R₁₄)(NR₁₅R₁₆), 7,8-dimethyl-isoalloxazin-10-yl-C₁₋₈alkyl, or aryl wherein said aryl and alkyl are optionally substituted with —COOR₉;
(viii) R₁₂ is C₁₋₈alkyl (e.g., methyl, ethyl, t-Butyl);
(ix) R₁₃, R₁₄, R₁₅ and R₁₆ are independently selected from H, C₁₋₈alkyl, and —C₁₋₈alkyl-COOR₁₈ (e.g., —CH(methyl)-COOR₁₈);
(x) R₁₈ is H or C₁₋₈alkyl (e.g., ethyl or t-butyl); in free, salt or prodrug form.

In still another embodiment, the invention relates to a compound of Formula I(v):

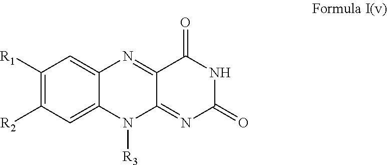

wherein
(i) R₁ is H, C₁₋₈ alkyl (e.g., methyl);
(ii) R₂ is H, halo (e.g., chloro), C₁₋₈alkyl (e.g., methyl or ethyl), C₁₋₈alkoxy (e.g., methoxy or ethoxy), —N(R₄)(R₅);
(iii) R₃ is C₂alkyl-N(R₆)(R₇), C₁₋₂alkyl-C(O)N(R₆)(R₇), C₁₋₈alkyl-P(O)(OR₉)(OR₁₇), C₁₋₈alkyl-P(O)(OR₉)(NR₁₃R₁₄), C₁₋₈alkyl-P(O)(NR₁₃R₁₄)(NR₁₅R₁₆), C₁₋₈alkyl-C(O)OR₉, C₁₋₈alkyl-OR₁₀;
(iv) R₄ and R₅ are independently selected from H, C₃₋₇ cycloalkyl (e.g., cyclopropyl or cyclopentyl), and C₁₋₈alkyl (e.g., methyl, ethyl or 2,2-dimethylpropyl) wherein said alkyl is optionally substituted with one or more groups selected from —OR₁₁;
(v) R₆ and R₇ are independently selected from —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O)(OR₉)(NR₁₃R₁₄), P(O)(NR₁₃R₁₄)(NR₁₅R₁₆), 7,8-dimethyl-isoalloxazin-10-yl-C₁₋₈alkyl and aryl wherein said aryl and alkyl are optionally substituted with —COOR₉;
(vi) R₉ and R₁₇ are independently selected from H, C₁₋₈alkyl (e.g., methyl, ethyl, n-butyl or t-butyl), —C₁₋₄alkyl-OC(O)R₁₂;
(vii) R₁₀ is H, —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl wherein said aryl and alkyl are optionally substituted with —COOR₉;
(viii) R₁₂ is C₁₋₈alkyl (e.g., methyl, ethyl, t-Butyl);
(ix) R₁₃, R₁₄, R₁₅ and R₁₆ are independently selected from H, C₁₋₈alkyl, and —C₁₋₈alkyl-COOR₁₈ (e.g., —CH(methyl)-COOR₁₈);
(x) R₁₈ is H or C₁₋₈alkyl (e.g., ethyl or t-butyl);
in free, salt or prodrug form.

In yet another embodiment, the invention provides a compound of Formula I, wherein R₃ is C₁₋₈ alkyl (e.g., n-butyl, n-pentyl, n-propyl, n-hexyl or n-heptyl) substituted with one or more groups selected from —OP(O)(OR₉)(OR₁₇), —OP(O)(OR₉)(NR₁₃R₁₄), —OP(O)(NR₁₃R₁₄)₁₅R₁₆), —P(O)(OR₉)(OR₁₇)₅—P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇) and the other substituents are defined in Formula I or any of 1.1-1.212, in free, salt or prodrug form.

In another embodiment, the invention provides a compound of formula I or I(i)-I(v) as follows:
1.213. Formula I, or any of 1.1-1.212, e.g., any of 1.1-1.109, 1.112, 1.130, 1.131, 1.132, 1.137, 1.142, 1.145, 1.150, 1.154, 1.157, 1.158, 1.212, or Formula I(i), I(ii), wherein R₃ is C₁₋₈ alkyl (e.g., methyl, ethyl, n-butyl, n-pentyl, n-propyl, n-hexyl or n-heptyl), wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇);
1.214. Formula I, or any of 1.1-1.212, e.g., any of 1.1-1.109, 1.112, 1.130, 1.131, 1.132, 1.137, 1.142, 1.145, 1.150, 1.154, 1.157, 1.158, 1.212, or any of Formulae I(i)-I(iii), 2.10, wherein R₃ is C₁₋₈ alkyl (e.g., methyl, ethyl, n-butyl, n-pentyl, n-propyl, n-hexyl or n-heptyl), wherein the alkyl group is optionally substituted with one or more groups selected from —P(O)(OR₉)(OR₁₇), —P(O)(OR₉)(NR₁₃R₁₄), —P(O)(NR₁₃R₁₄)(NR₁₅R₁₆), —C(O)OR₉, —OR₁₀, —C(O)N(R₆)(R₇), and —N(R₆)(R₇);
1.215. Formula I, or any of 1.1-1.212, e.g., any of 1.1-1.109, 1.112, 1.130, 1.131, 1.132, 1.137, 1.142, 1.145, 1.150, 1.154, 1.157, 1.158, 1.212, or any of Formulae I(i)-I(iv) or 2.10-1.214, wherein R₃ is C₁₋₈alkyl-N(R₆)(R₇), C₁₋₈alkyl-C(O)N(R₆)(R₇), C₁₋₈alkyl-P(O)(OR₉)(OR₁₇), C₁₋₈alkyl-P(O)(OR₉)(NR₁₃R₁₄), C₁₋₈alkyl-P(O)(NR₁₃R₁₄)(NR₁₅R₁₆), C₁₋₈alkyl-C(O)OR₉, C₁₋₈alkyl-OR₁₀;
1.216. Formula I, or any of 1.1-1.212, e.g., any of 1.1-1.109, 1.112, 1.130, 1.131, 1.132, 1.137, 1.142, 1.145, 1.150, 1.154, 1.157, 1.158, 1.212, or any of Formulae I(i)-I(v) or 2.10-1.215, wherein R₃ is R₃ is C₂alkyl-N(R₆)(R₇), C₁₋₂alkyl-C(O)N(R₆)(R₇), C₁₋₈alkyl-P(O)(OR₉)(OR₁₇), C₁₋₈alkyl-P(O)(OR₉)(NR₁₃R₁₄), C₁₋₈alkyl-P(O)(NR₁₃R₁₄)(NR₁₅R₁₆), C₁₋₈alkyl-C(O)OR₉, C₁₋₈alkyl-OR₁₀;
1.217. any of Formulae 2.10-1.216, wherein R₃ is C₁₋₈alkyl (e.g., ethyl) substituted with —N(R₆)(R₇);
1.218. any of formulae 2.10-1.216, wherein R₃ is C₁₋₈alkyl (e.g., methyl) substituted with —C(O)N(R₆)(R₇);
1.219. any of formulae 2.12-1.218, wherein R₆ and R₇ are independently selected from H, C₁₋₈alkyl (e.g., methyl, ethyl, n-propyl, n-butyl), —C(O)OR₉, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-C₁₋₈alkyl and aryl wherein said aryl and alkyl are optionally substituted with —COOR₉;
1.220. any of formulae 2.12-1.218, wherein R₆ and R₇ are independently selected from R₆ and R₇ are independently selected from —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), 7,8-dimethyl-isoalloxazin-10-yl-C₁₋₈alkyl and aryl wherein said aryl and alkyl are optionally substituted with —COOR₉;
1.221. any of formulae 2.12-1.218, wherein R₆ and R₇ are independently selected from —C₁₋₈alkyl-COOR₉ (e.g., -methyl-COOR₉, -ethyl-COOR₉, -propyl-COOR₉, hexyl-COOR₉), wherein said alkyl is optionally substituted with —COOR₉ (e.g., —C(H)(COOR₉)—CH₂CH₂—COOR₉ or —C(H)(COOR₉)—CH₂—COOR₉);
1.222. any of formulae 2.12-1.218, wherein R₆ and R₇ are independently selected from R₆ and R₇ are independently selected from —C₁₋₈alkyl-C(O)OR_{S}, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O)(OR₉)(NR₁₃R₁₄), P(O)(NR₁₃R₁₄)(NR₁₅R₁₆), 7,8-dimethyl-isoalloxazin-10-yl-C₁₋₈alkyl, or aryl, wherein said aryl and alkyl are optionally substituted with —COOR₉;
1.223. any of formulae 2.12-1.218, wherein R₆ and R₇ are independently —C₁₋₈alkyl-C(O)OR₉ wherein said alkyl is optionally substituted with —COOR₉;
1.224. any of formulae 2.12-1.218, wherein R₆ and R₇ are independently —C₁₋₈alkyl(amine)-C(O)OR₉ (e.g., —CH₂CH₂CH(NH₂)COOH);
1.225. any of formulae 2.12-1.218, wherein R₆ and R₇ are independently —C₁₋₈alkyl-P(O)(NR₁₃R₁₄)(NR₁₅R₁₆);
1.226. any of formulae 2.12-1.218, wherein R₆ and R₇ are independently —C₁₋₈alkyl-P(O)(OR₉)(OR₁₇);
1.227. any of formulae 2.12-1.218, wherein R₆ and R₇ are independently 7,8-dimethyl-isoalloxazin-10-yl-C₁₋₈alkyl;
1.228. any of formulae 2.12-1.218, wherein R₆ and R₇ are independently aryl wherein said aryl is optionally substituted with —COOR₉;
1.229. any of formulae 2.12-1.228, wherein R₁₃, R₁₄, R₁₅ and R₁₆ are independently selected from H, C₁₋₈alkyl, and —C₁₋₈alkyl-COOR₁₈ (e.g., —CH(methyl)-COOR₁₈, —CH(isopropyl)-COOR₁₈, —CH(isobutyl)-COOR₁₈, —CH(sec-butyl)-COOR₁₈), wherein the alkyl group of C₁₋₈alkyl-COOR₁₈ is optionally substituted with hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH);
1.230. any of formulae 2.12-1.228, wherein R₁₃, R₁₄, R₁₅ and R₁₆ are independently H, C₁₋₈alkyl;
1.231. any of formulae 2.12-1.228, wherein R₁₃, R₁₄, R₁₅ and R₁₆ are independently —CH(CH₃)COOR₁₈;
1.232. Formula I, or any of 1.1-1.212, e.g., any of 1.1-1.109, 1.157, 1.212, or any of Formulae I(i)-I(iv), 1.213-1.222, wherein R₃ is —C₁₋₈alkyl-C(O)OR₉;
1.233. Formula I, or any of 1.1-1.212, e.g., any of 1.1-1.109, 1.112, 1.158, 1.212, or any of Formulae I(i)-I(v) or 2.10-1.222, wherein R₃ is —C₁₋₈alkyl-OR₁₀;
1.234. formula 1.233, wherein R₁₀ is selected from H, C₁₋₈alkyl (e.g., methyl or ethyl), —C₁₋₈alkyl-OR₁₁, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl, or aryl wherein said aryl and alkyl are optionally substituted with —COOR₉, or —C₁₋₄alkyl-OC(O)R_{12;}
1.235. formula 1.233, wherein R₁₀ is selected from —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl wherein said aryl and alkyl are optionally substituted with —COOR₉;
1.236. formula 1.233, wherein R₁₀ is —C₁₋₈alkyl-C(O)OR₉;
1.237. formula 1.233, wherein R₁₀ is -propyl-COOR₉
1.238. any of Formulae I(i)-I(iv), 2.10-1.236, wherein R₉ is ethyl;
1.239. any of Formulae I(i)-I(iv), 2.10-1.236, wherein R₉ is t-butyl;
1.240. any of Formulae I(i)-I(iv), 2.10-1.236, wherein R₉ is isopropyl;
1.241. any of Formulae I(i)-I(iv), 2.10-1.236, wherein R₉ is n-butyl;
1.242. any of Formulae I(i)-I(iv), 2.10-1.241, wherein R₁₈ is ethyl;
1.243. any of Formulae I(i)-I(iv), 2.10-1.241, wherein R₉ and R₁₇ are independently H or —CH₂—OC(O)R₁₂;
1.244. any of Formulae I(i)-I(iv), 2.10-1.237, wherein R₉ and R₁₇ are independently H,
1.245. any of Formulae I(i)-I(iv), 2.10-1.237, wherein R₉ and R₁₇ are independently —CH₂—OC(O)R₁₂;
1.246. any of Formulae I(i)-I(iv), 2.10-1.237, wherein R₉ and R₁₇ are independently C₁₋₈alkyl (e.g., methyl, ethyl, n-butyl or t-butyl);
1.247. any of Formulae I(i)-I(iv), 2.10-1.237, wherein R₉ and R₁₇ are independently phenyl and Bn wherein said phenyl and Bn are optionally substituted with one or more halo or C₁₋₄alkoxy (e.g., 3-chloro-phenylmethyl, 3-fluoro-phenylmethyl, 4-methoxy-3-fluorophenylmethyl);
1.248. any of the preceding formulae, wherein R₃ is selected from any of the following:

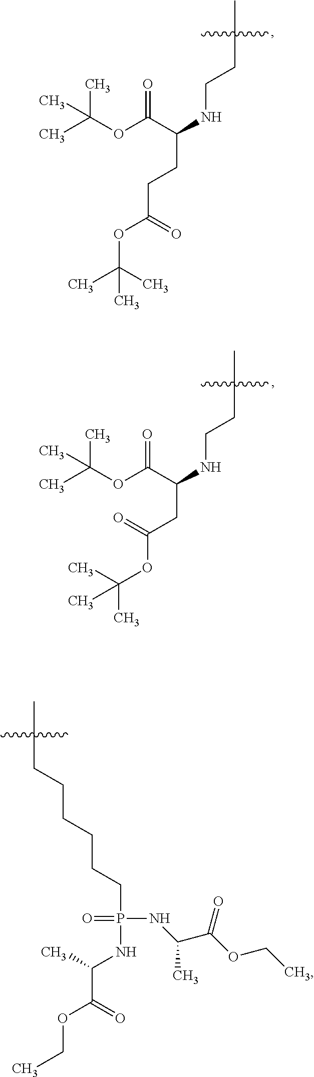

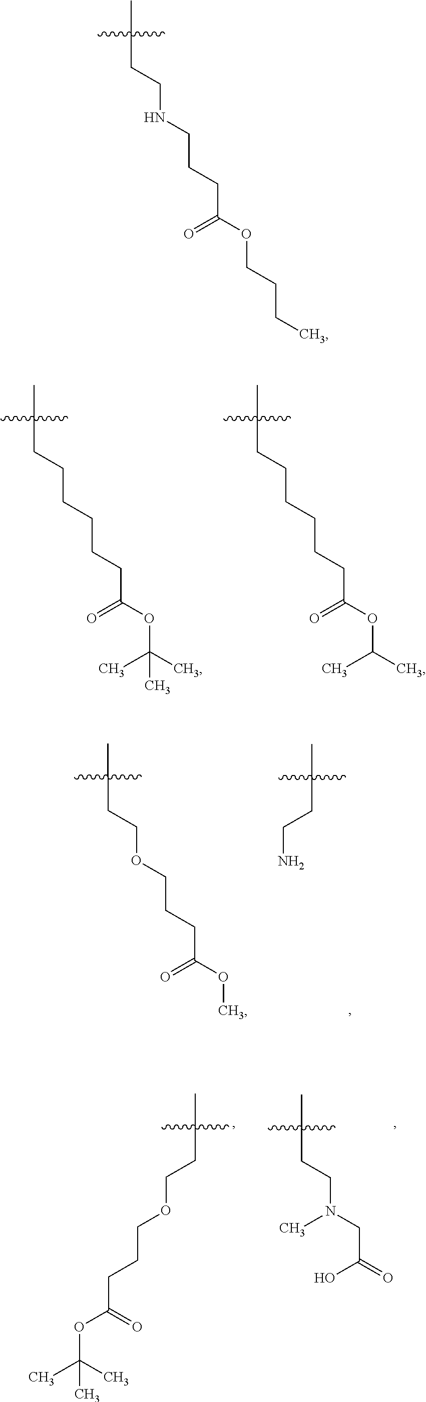

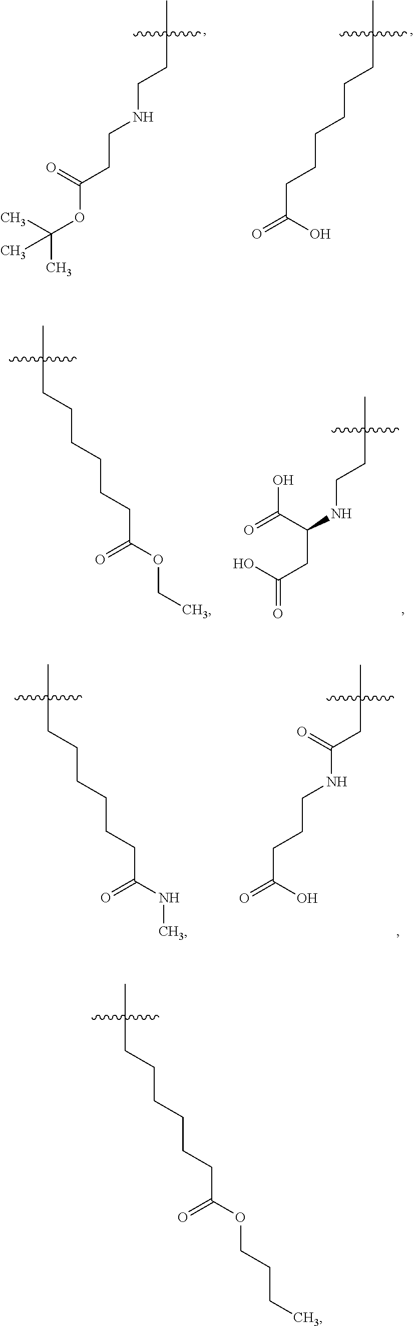

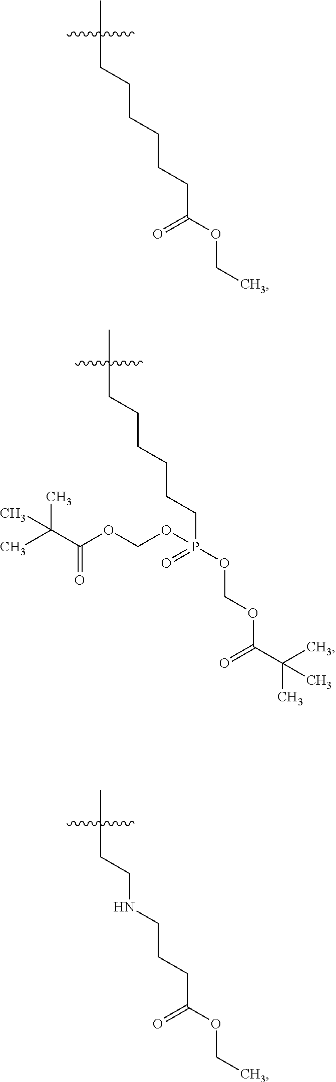

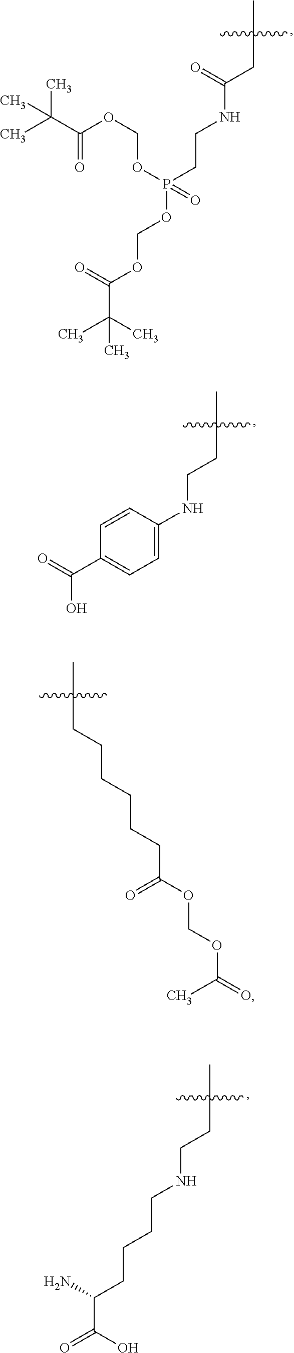

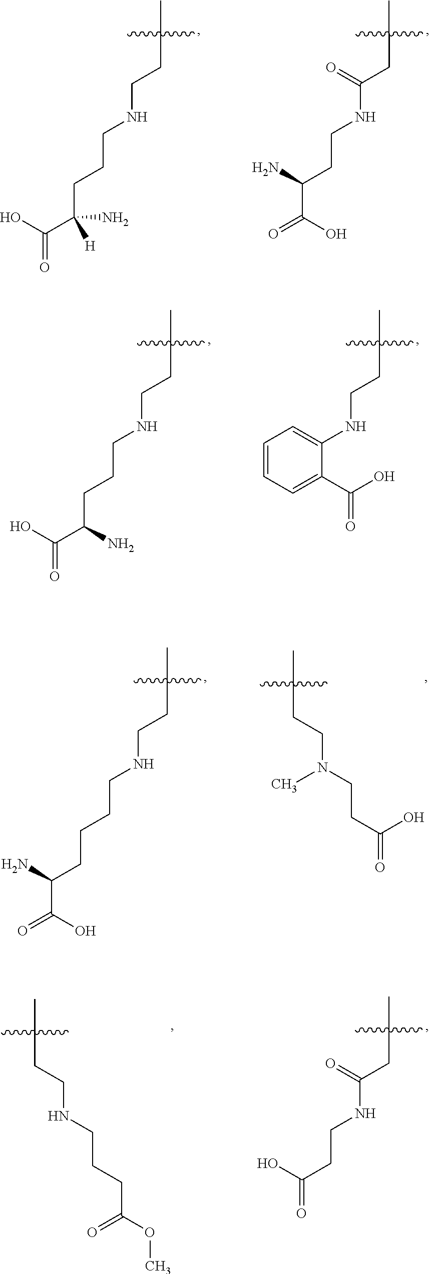

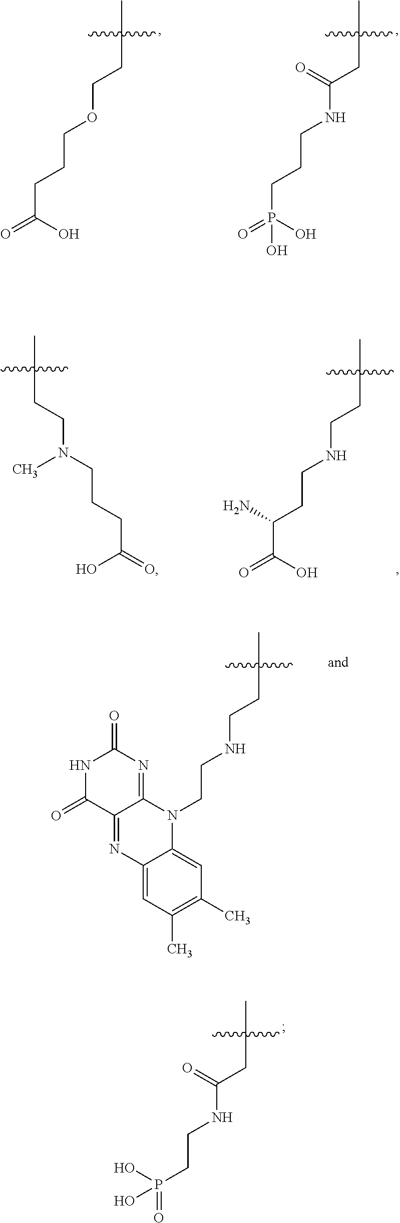

1.249. formulae 1.248, wherein R₆ and R₇ are independently selected from —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl, wherein said aryl and alkyl are optionally substituted with —COOR₉;
1.250. Formula I, or any of 1.1-1.212, or any of formulae I(i)-I(iv), e.g., any of 1.213-1.249, wherein R₂ is H, halo (e.g., chloro), C₁₋₈alkyl (e.g., methyl or ethyl), C₁₋₈alkoxy (e.g., methoxy or ethoxy), —N(R₄)(R₅), C₃₋₇cycloalkyl or C₄₋₇heterocycle (e.g., piperazinyl or pyrrolidinyl) wherein said heterocycle is optionally substituted with C₁₋₈alkyl (e.g., 4-methyl-piperazin-1-yl) or hydroxyC₁₋₈alkyl (e.g., 4-hydroxyethyl-piperazin-1-yl);
1.251. Formula I, or any of 1.1-1.212, or any of formulae I(i)-I(iv), e.g., any of 1.213-1.250, wherein R₂ is dimethylamino;
1.252. Formula I, or any of 1.1-1.212, or any of formulae I(i)-I(iv), e.g., any of 1.213-1.250, wherein R₂ is cyclopropylamino;
1.253. Formula I, or any of 1.1-1.212, or any of formulae I(i)-I(iv), e.g., any of 1.213-1.250, wherein R₂ is chloro;
1.254. Formula I, or any of 1.1-1.212, or any of formulae I(i)-I(iv), e.g., any of 1.213-1.250, wherein R₂ is H,
1.255. Formula I, or any of 1.1-1.212, or any of formulae I(i)-I(iv), e.g., any of 1.213-1.250, wherein R₂ is 2,2-dimethylpropyl;
1.256. Formula I, or any of 1.1-1.212, or any of formulae I(i)-I(iv), e.g., any of 1.213-1.250, wherein R₂ is hydroxyl-C₁₋₈alkylamino (e.g., hydroxyethylamino);
1.257. Formula I, or any of 1.1-1.212, or any of formulae I(i)-I(iv), e.g., any of 1.213-1.256, wherein R₁ is H, C₁₋₈ alkyl (e.g., methyl) or C₃₋₇ cycloalkyl;
1.258. Formula I, or any of 1.1-1.212, or any of formulae I(i)-I(iv), e.g., any of 1.213-1.256, wherein R₁ is H, C₁₋₈ alkyl (e.g., methyl);
1.259. Formula I, or any of 1.1-1.212, or any of formulae I(i)-I(iv), e.g., any of 1.213-1.258, selected from any of the following:

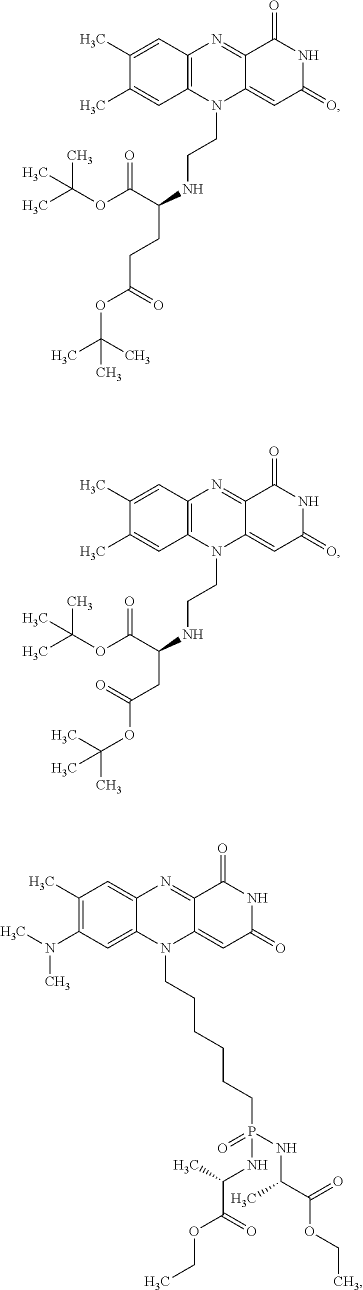

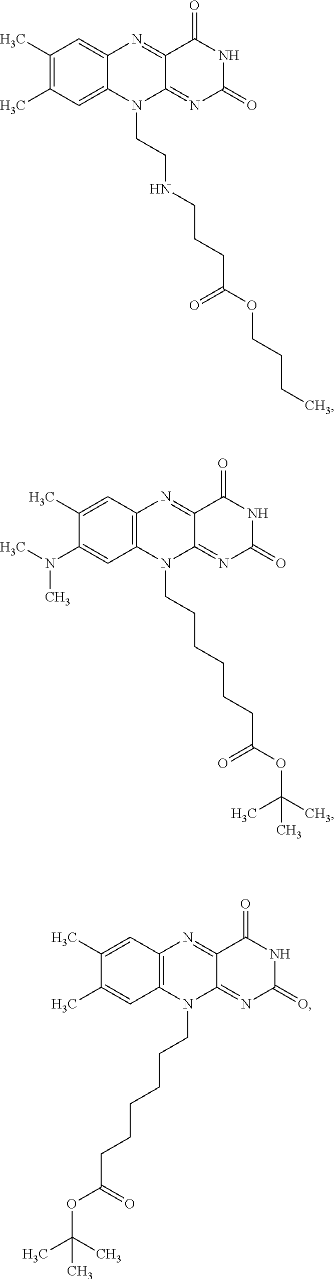

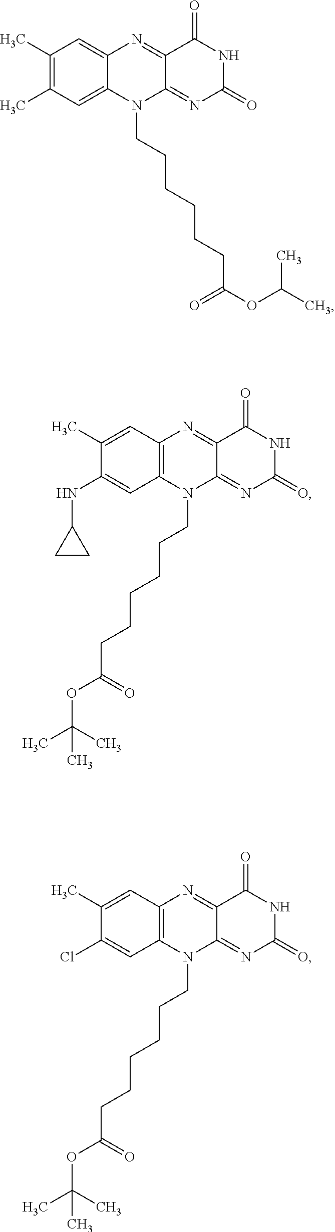

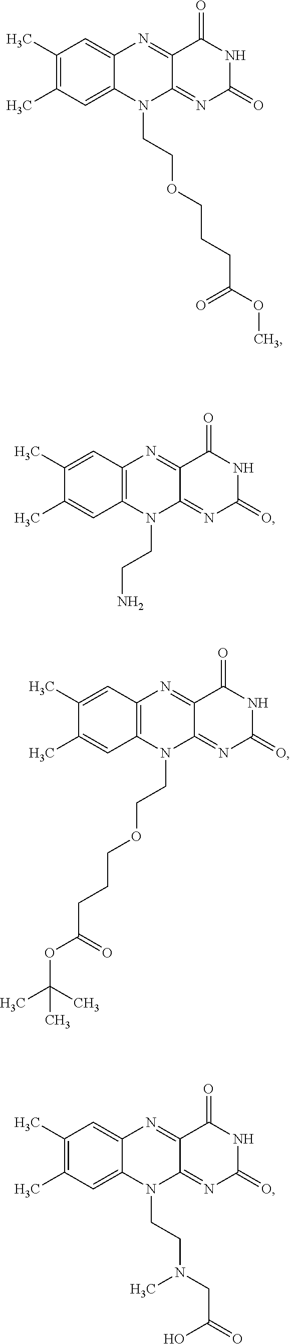

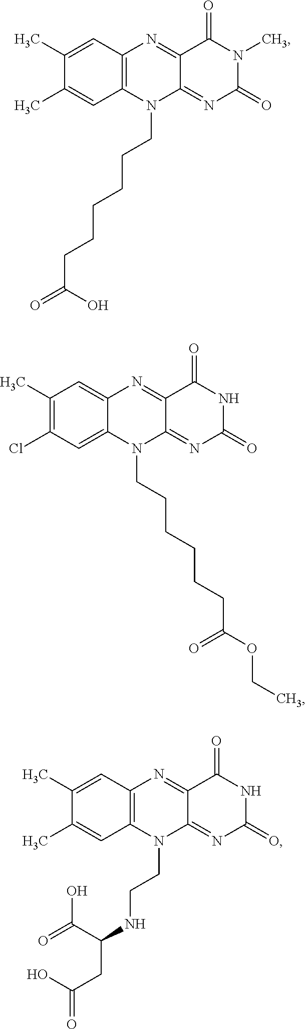

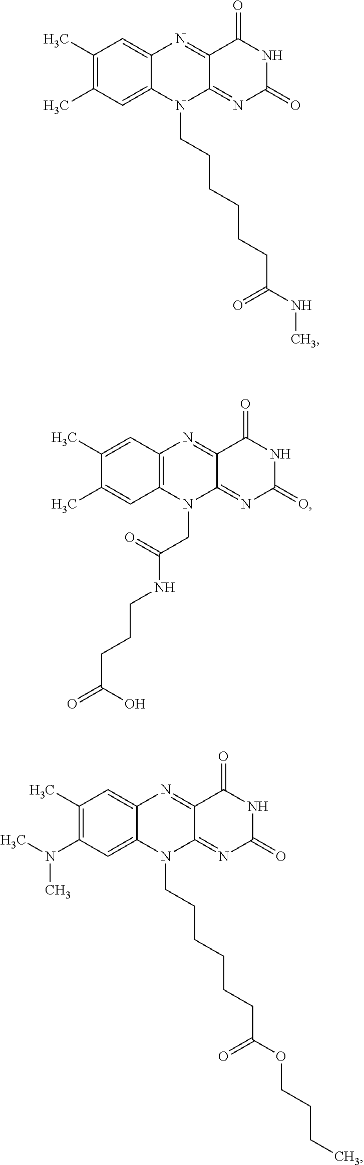

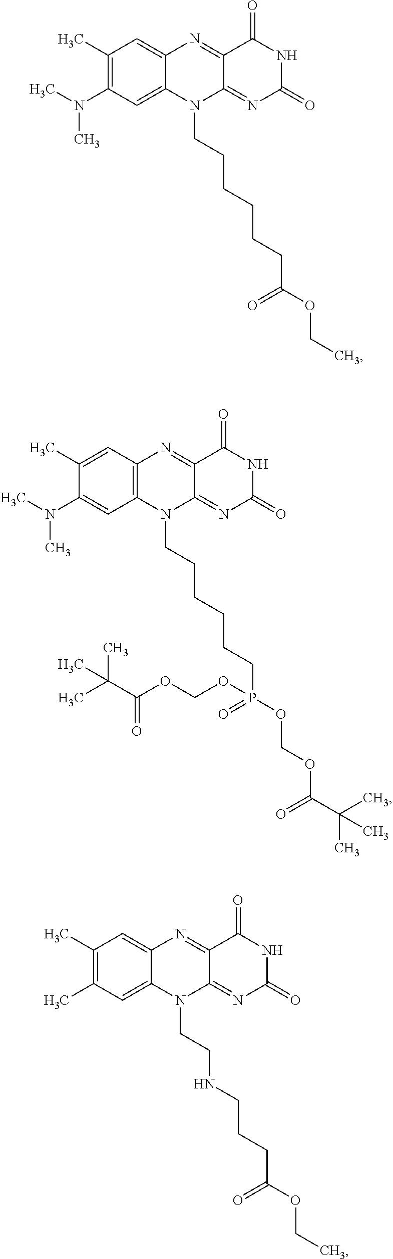

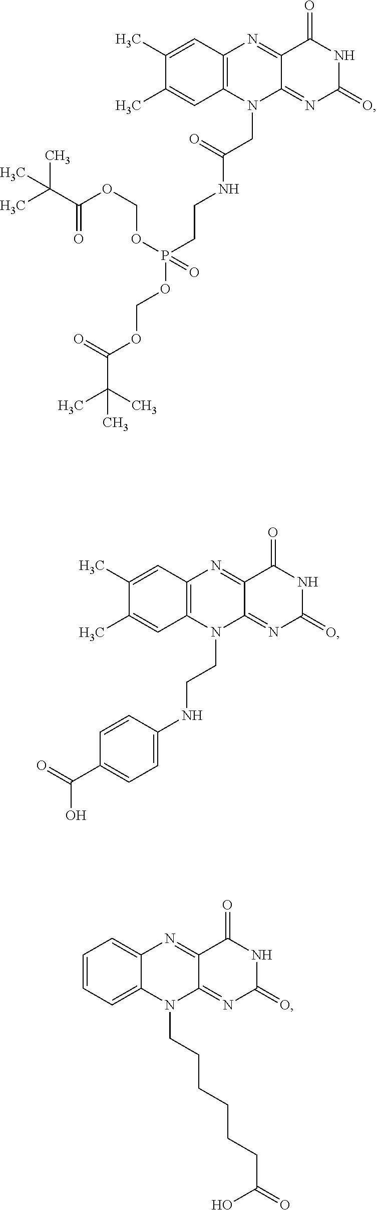

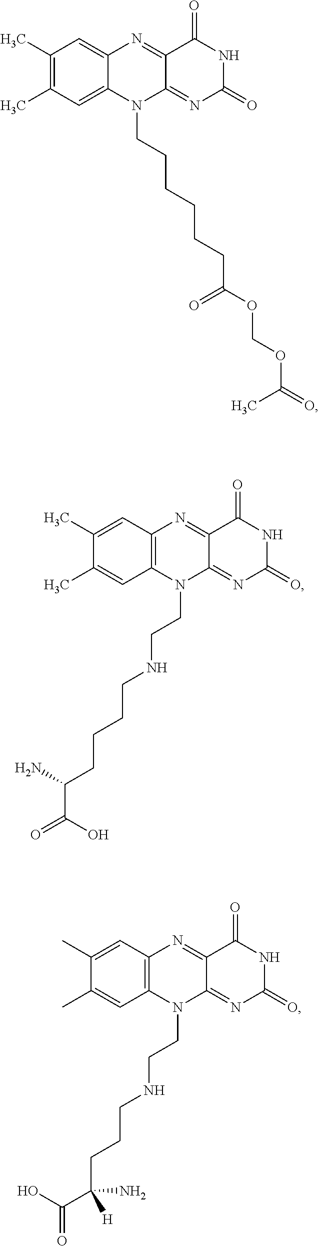

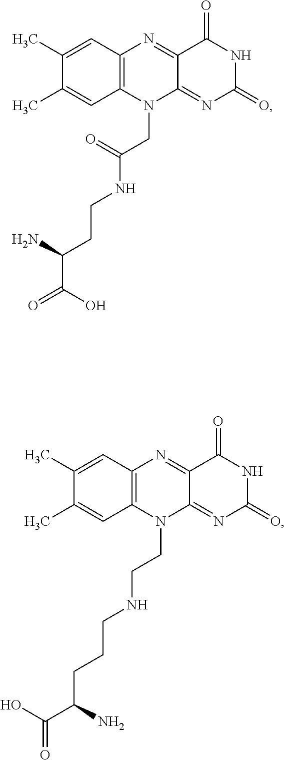

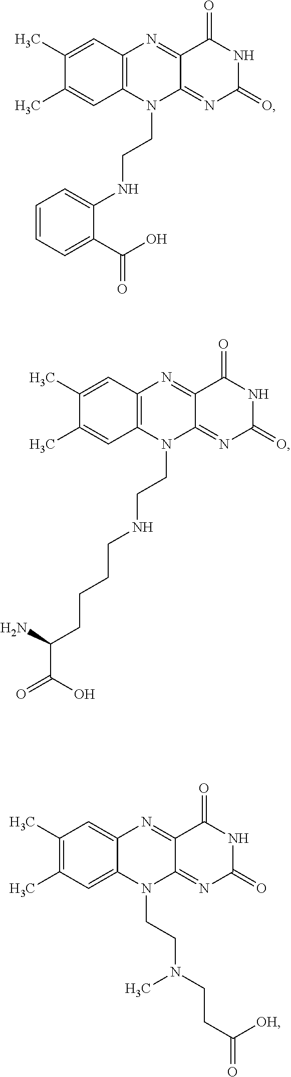

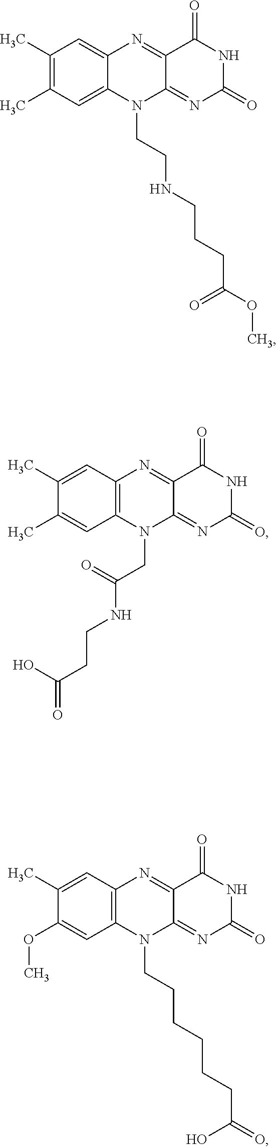

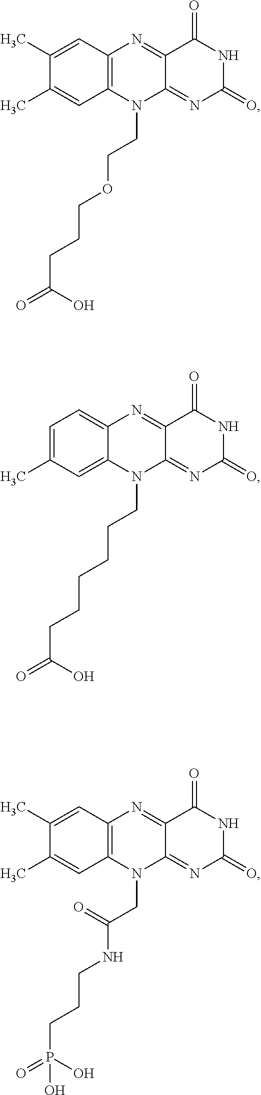

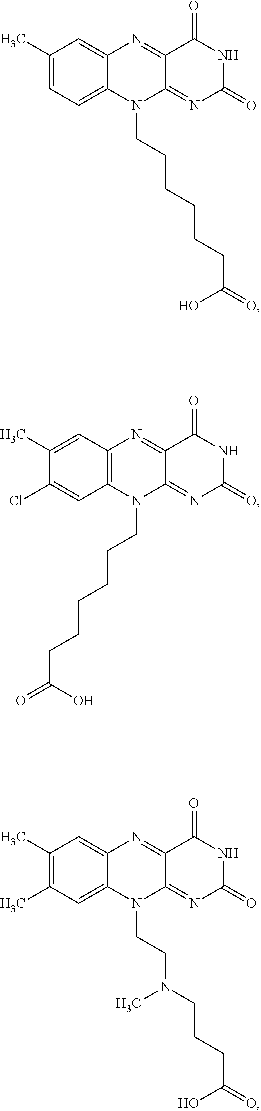

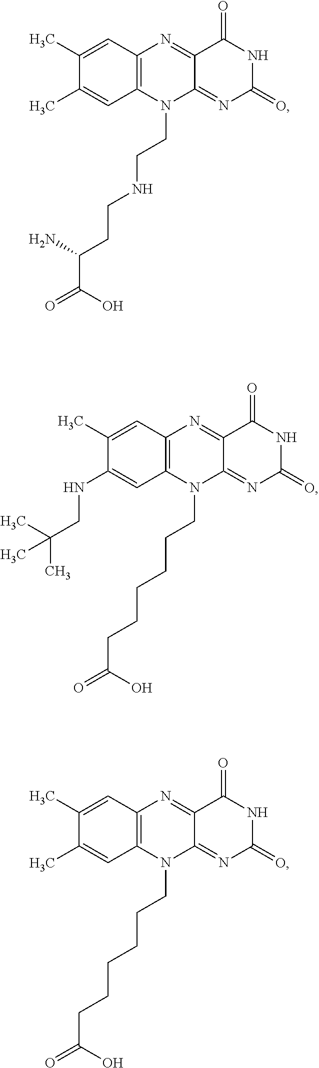

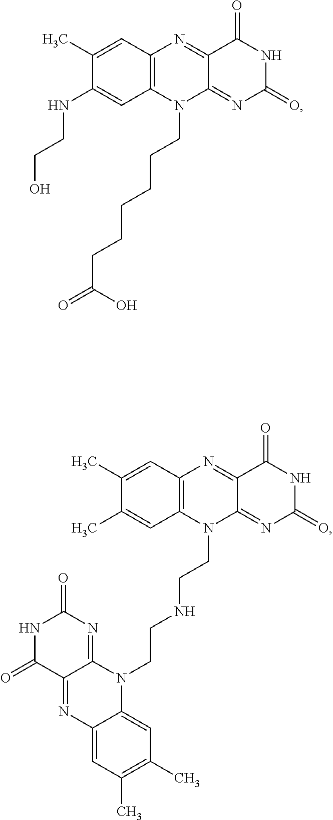

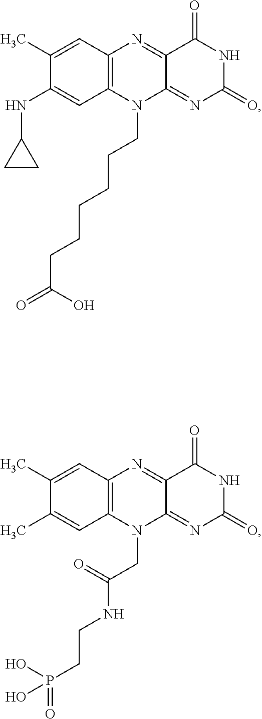

1.260. Formula I, or any of 1.1-1.259, selected from any of the following:

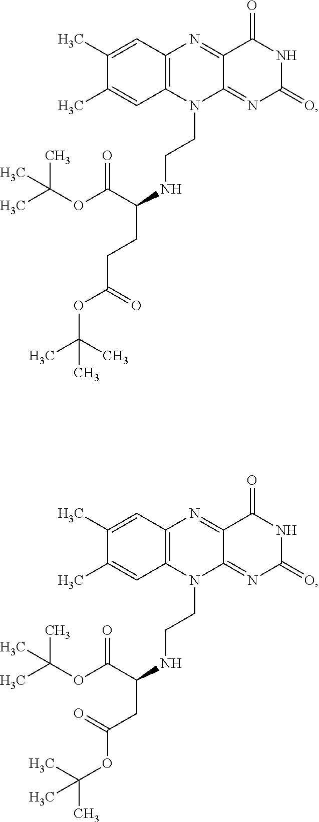

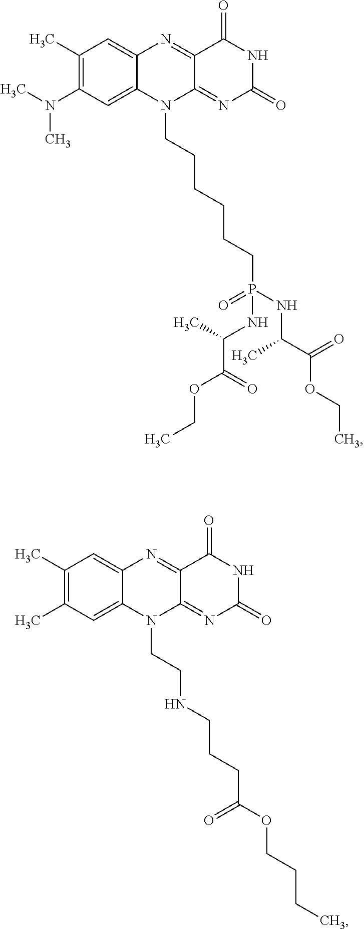

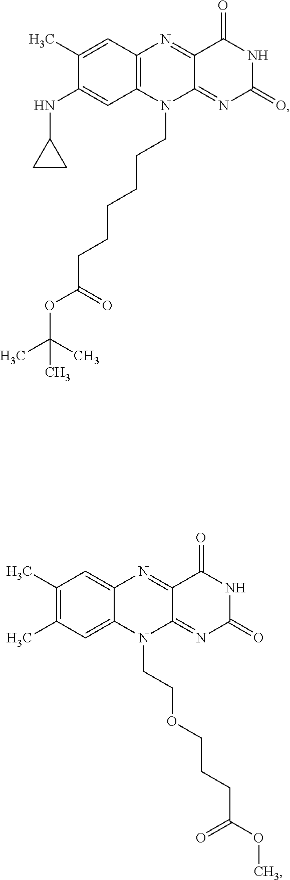

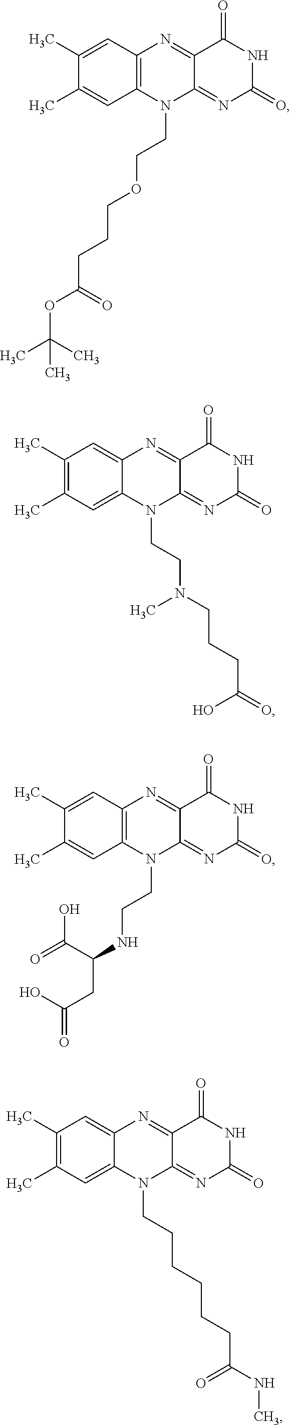

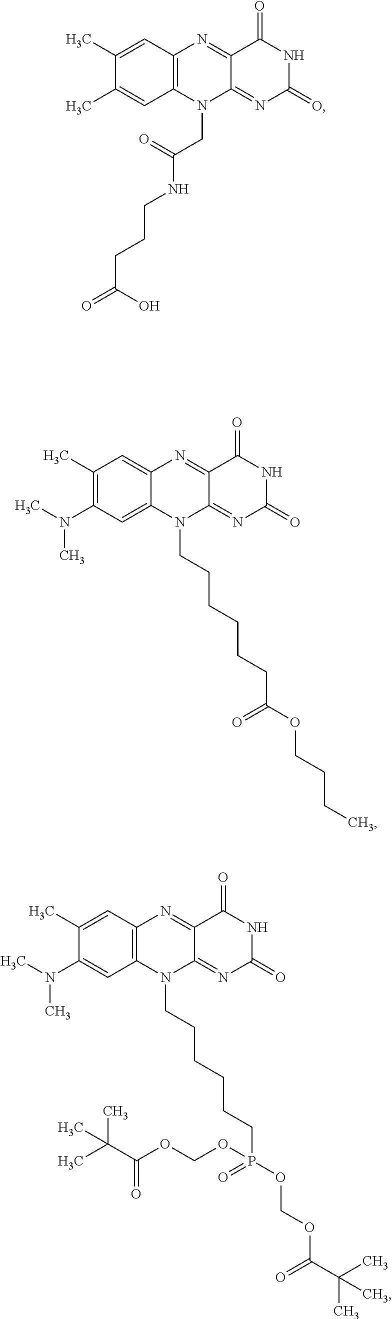

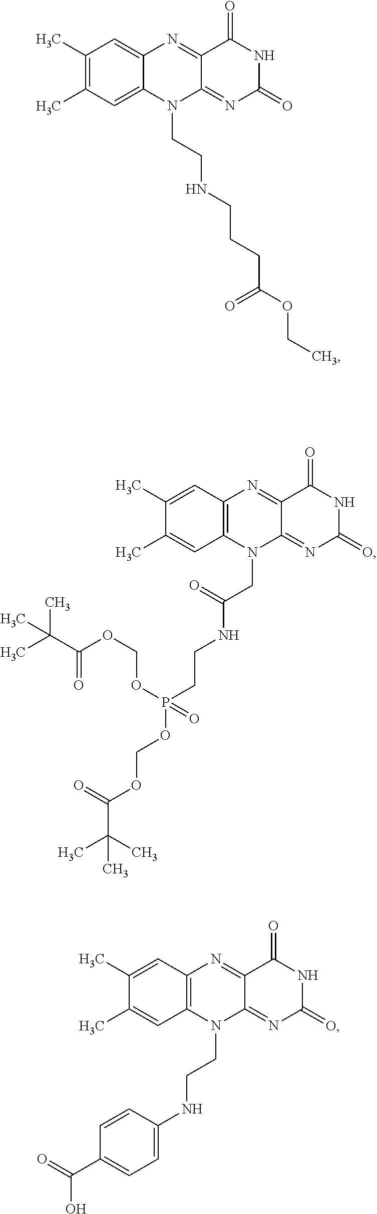

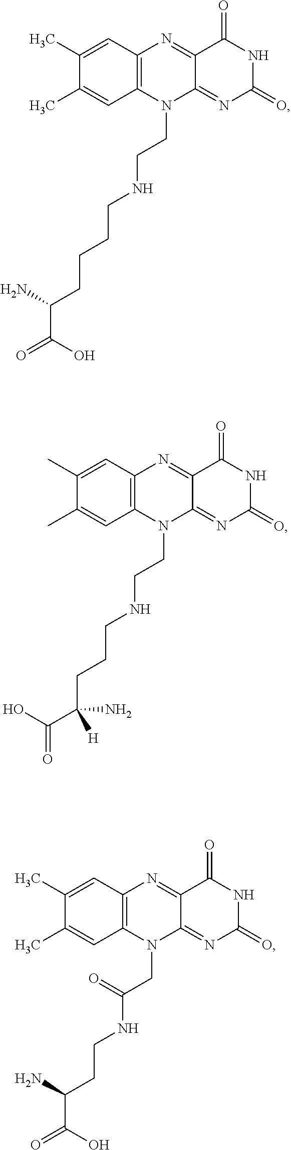

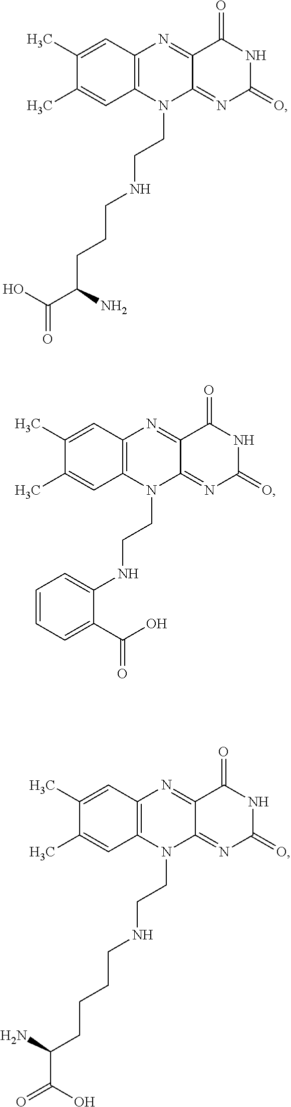

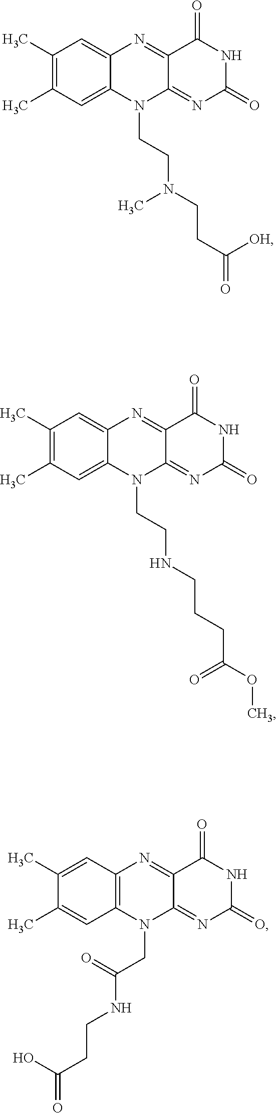

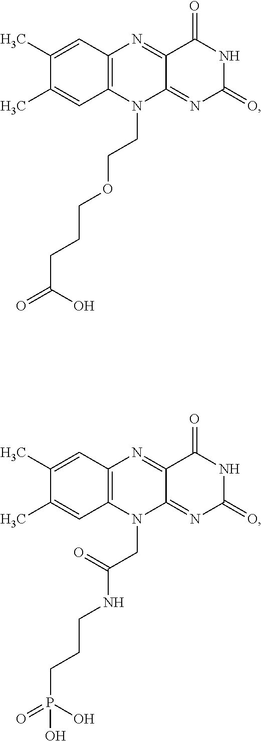

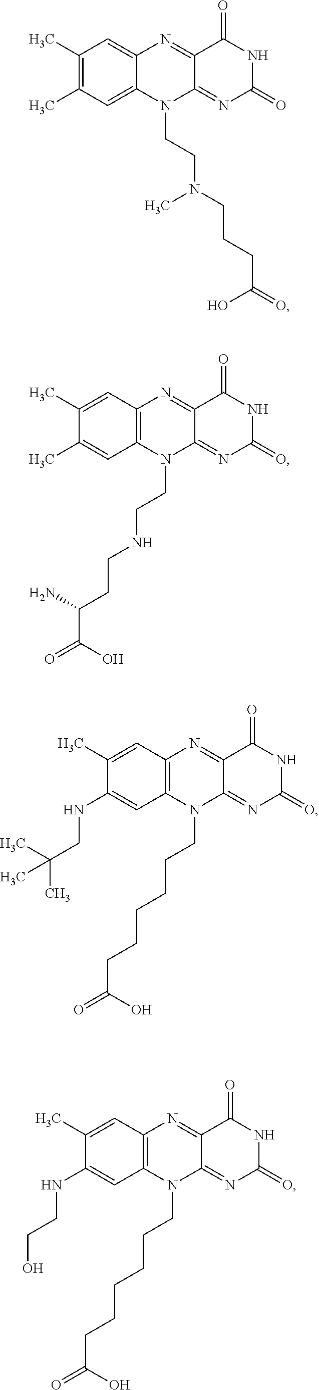

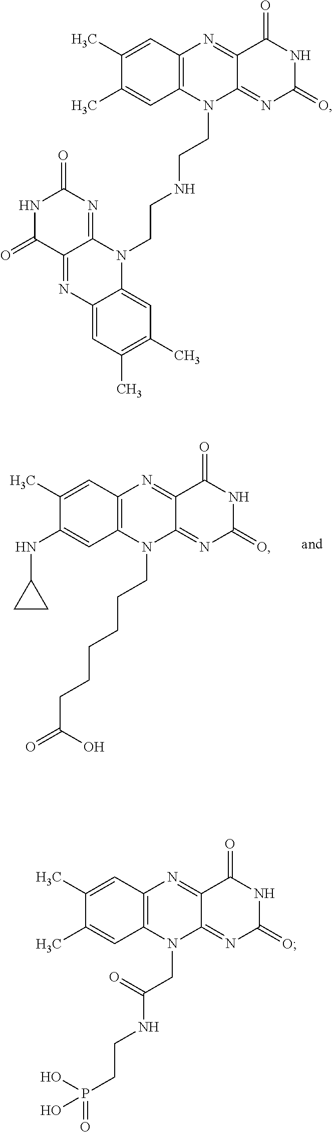

1.261. Formula I, or any of 1.1-1.259, selected from any of the following:

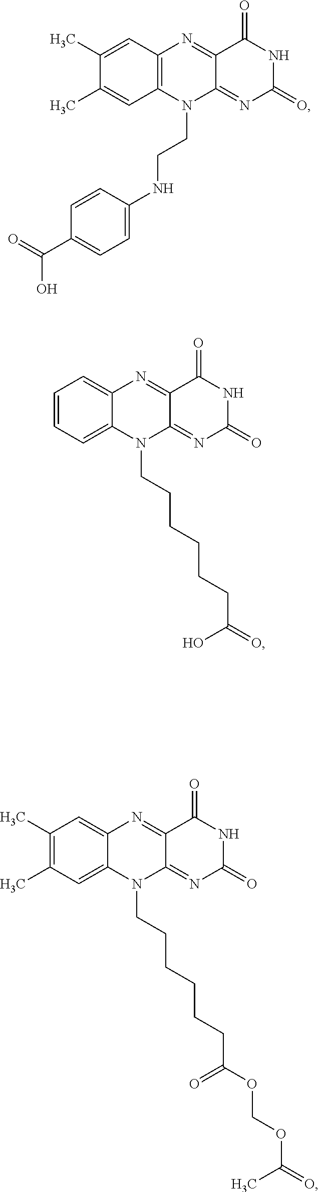

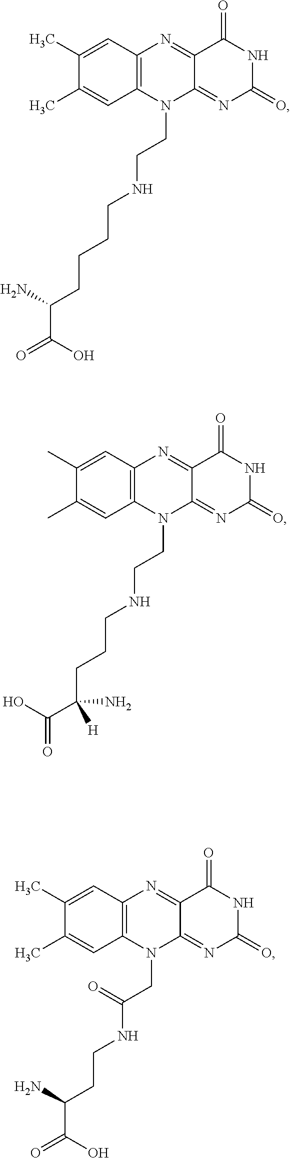

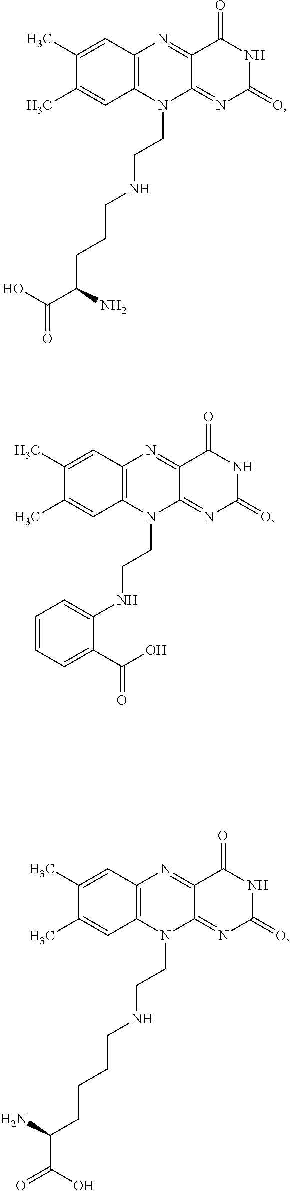

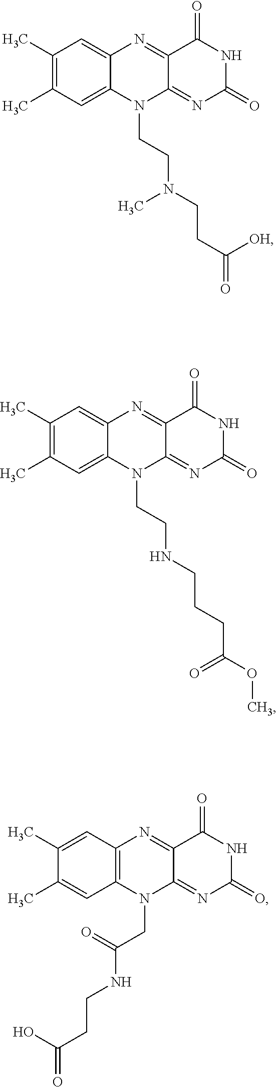

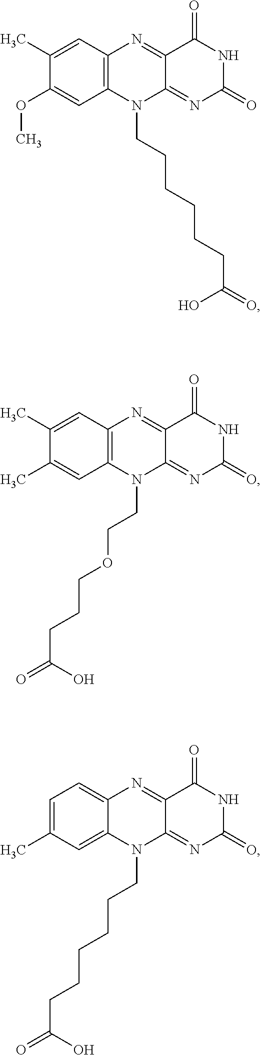

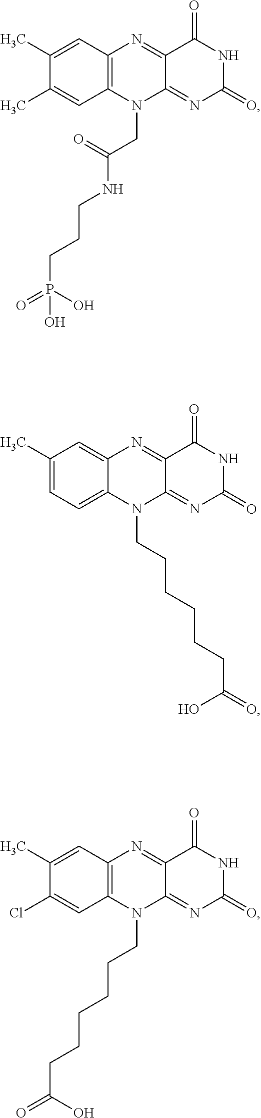

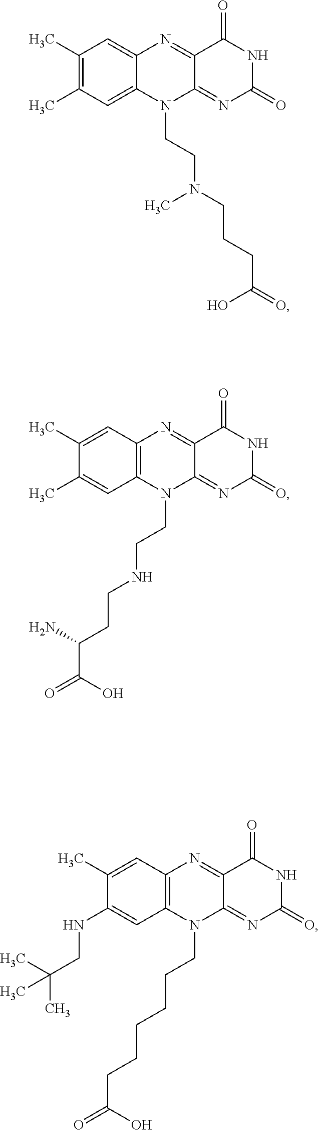

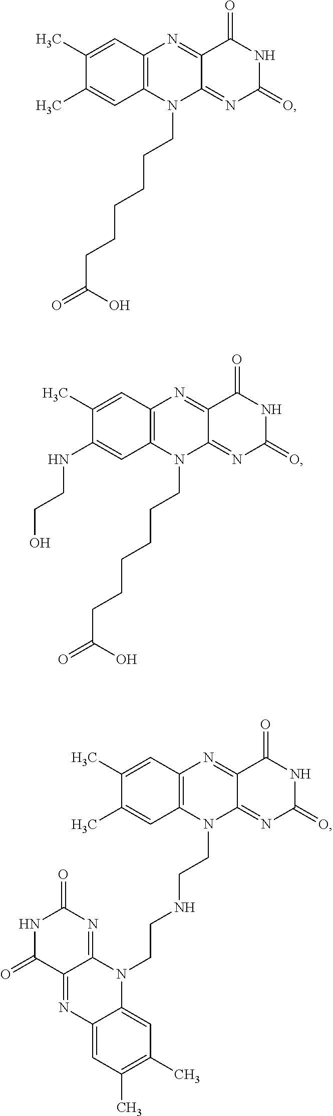

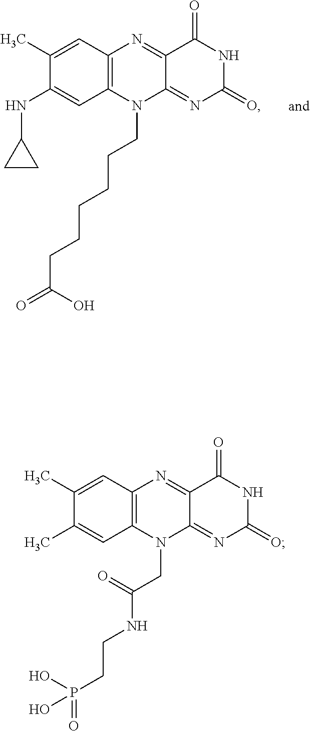

1.262. Formula I, or any of 1.1-1.259, selected from any of the following:

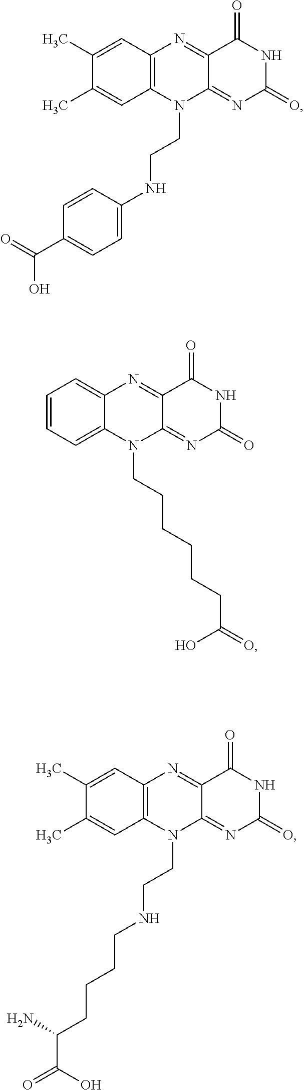

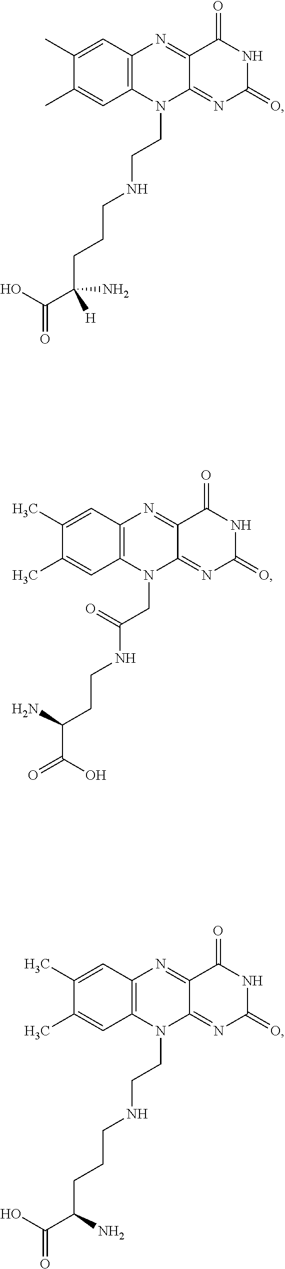

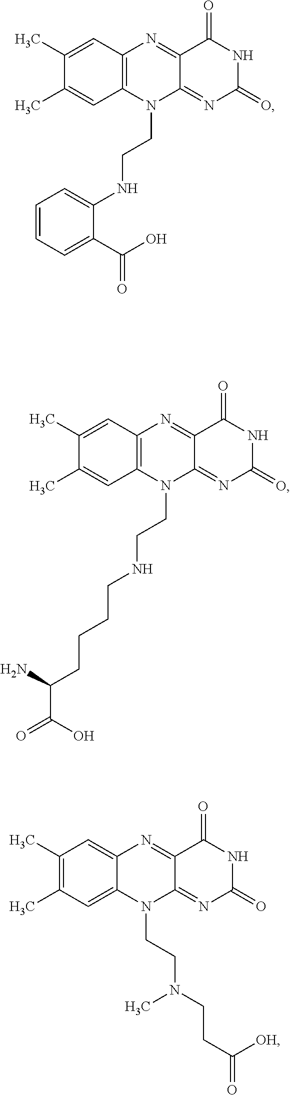

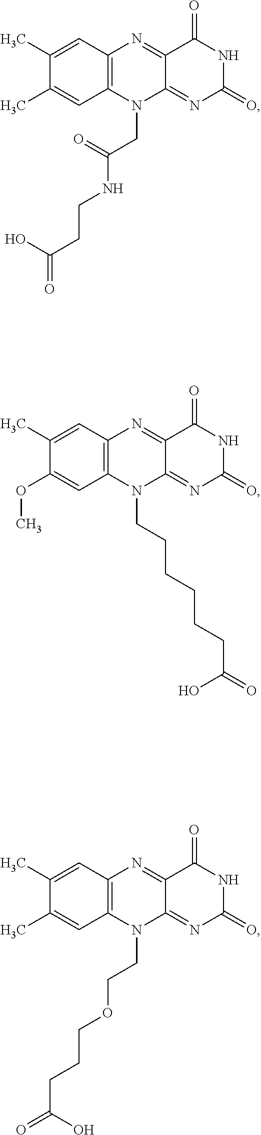

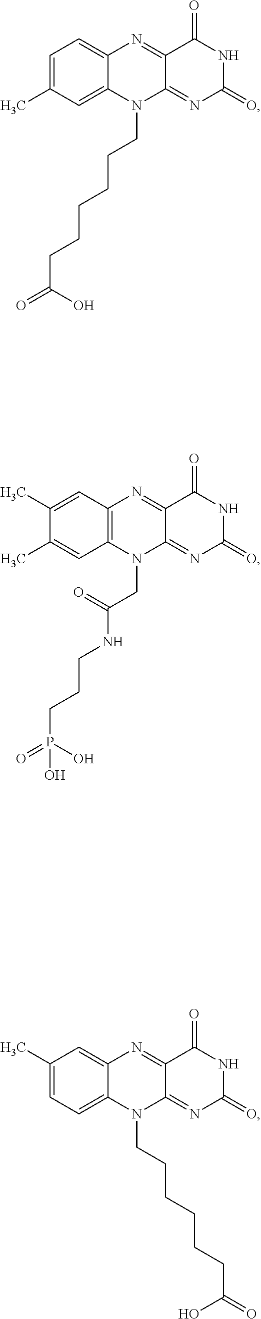

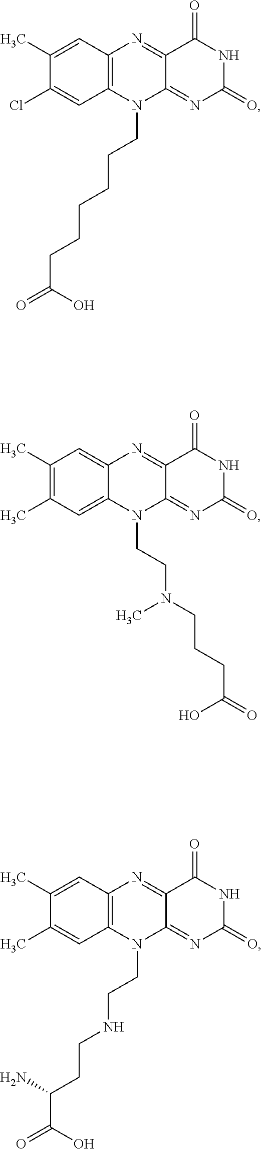

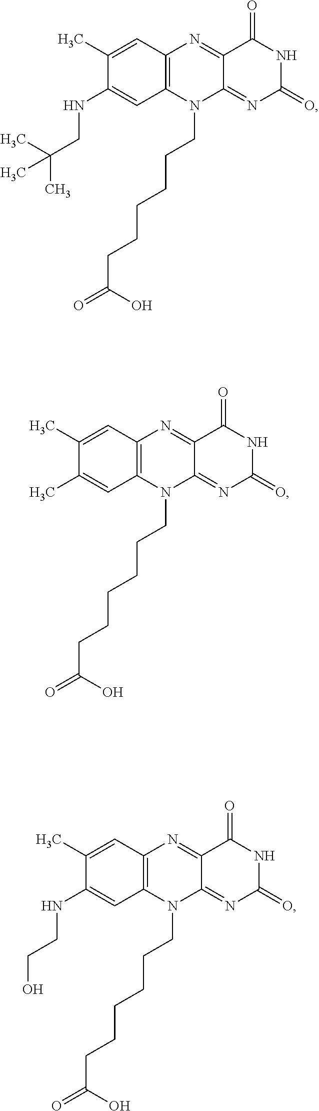

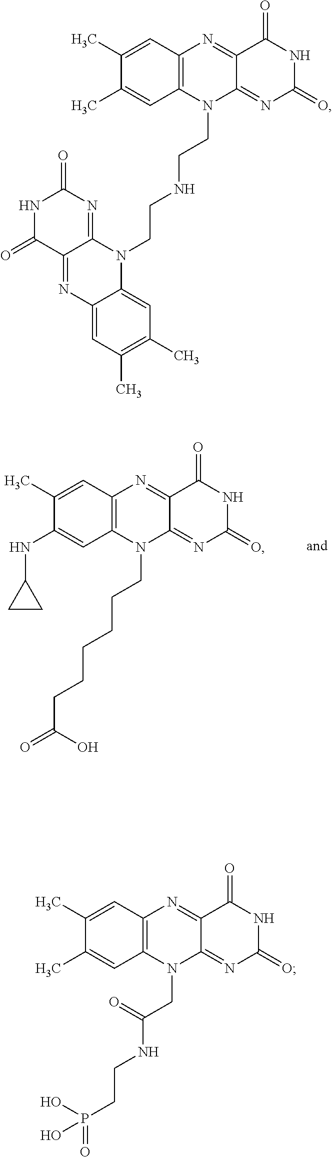

1.263. Formula I, or any of 1.1-1.259, selected from any of the following:

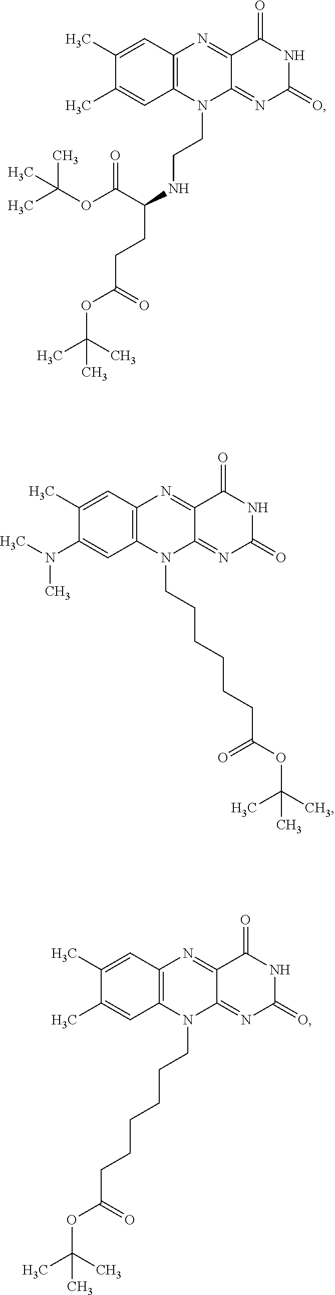

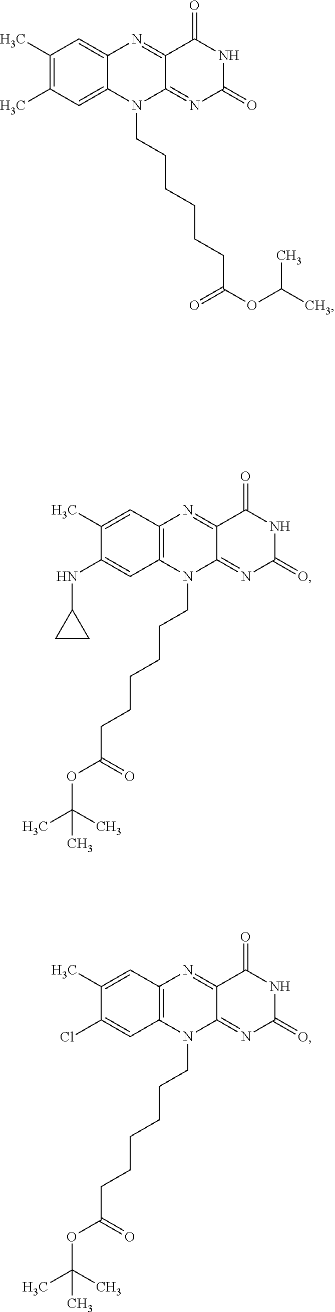

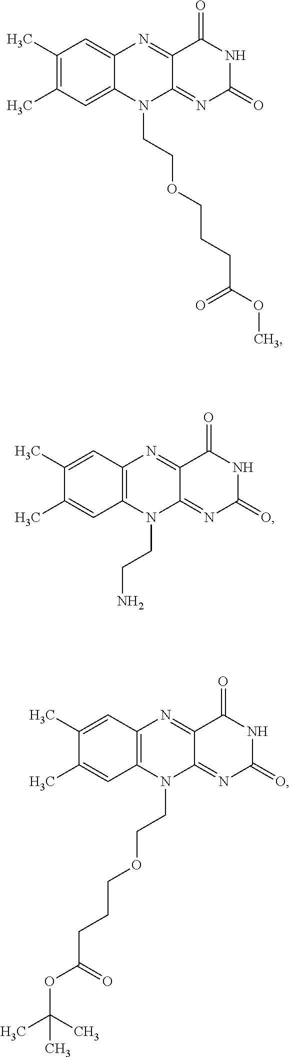

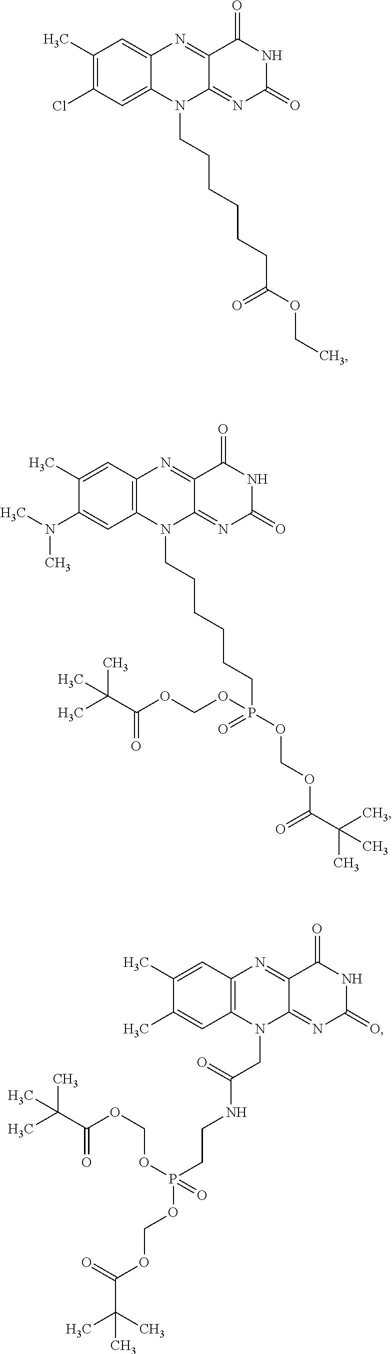

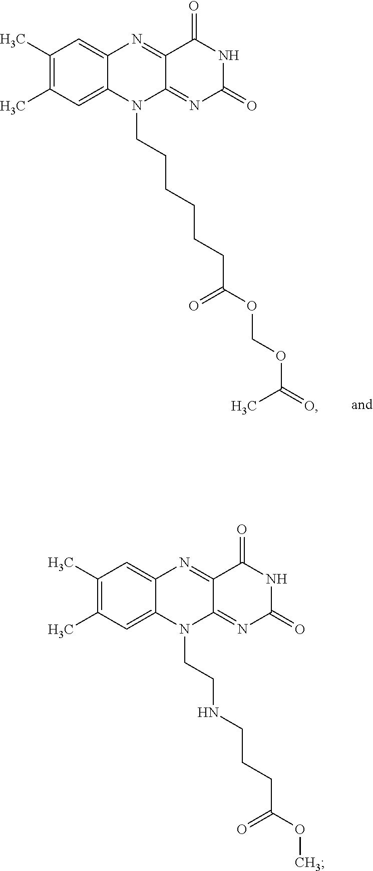

1.264. Formula I, or any of 1.1-1.259, selected from any of the following:

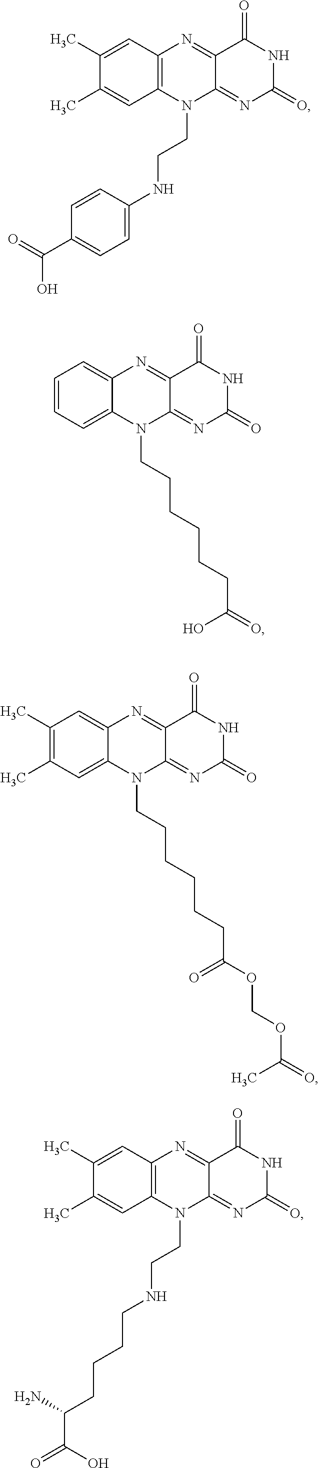

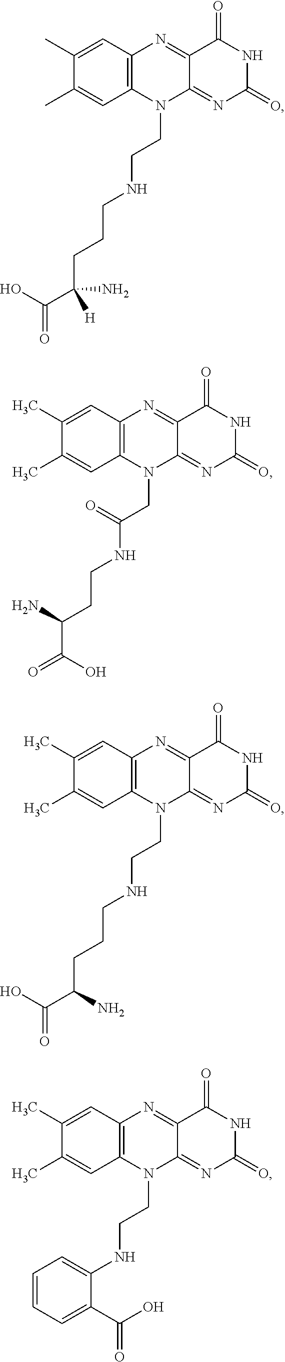

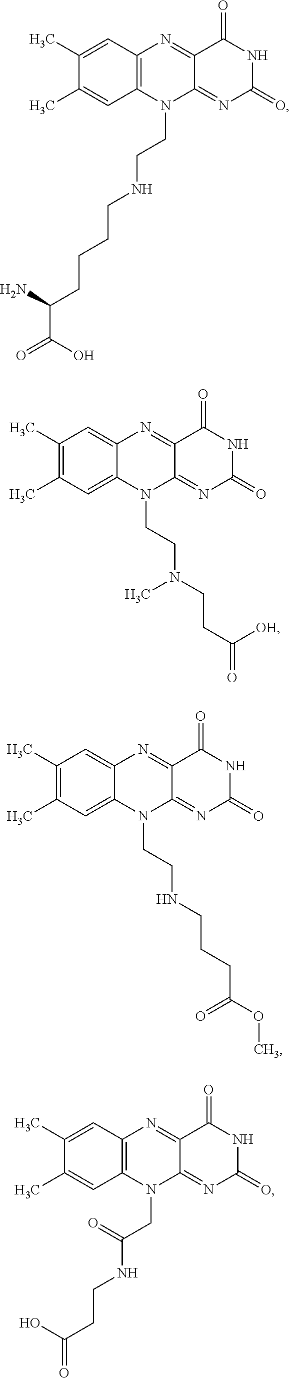

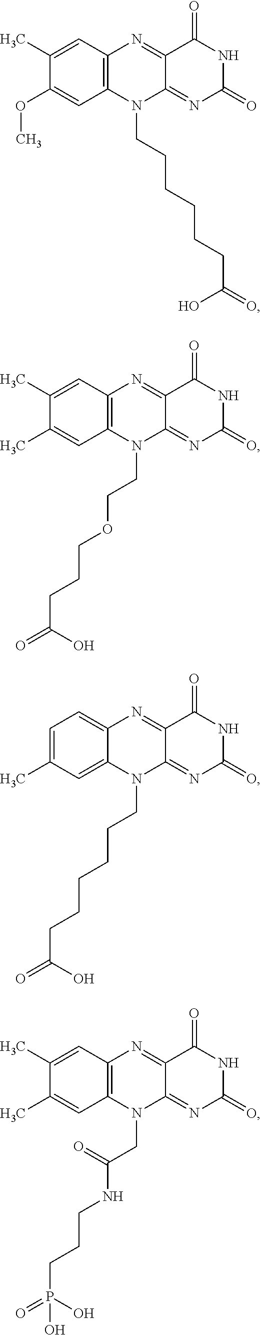

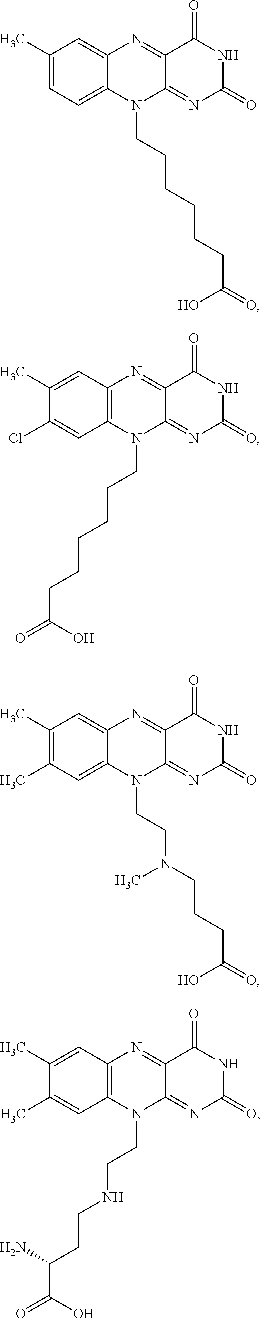

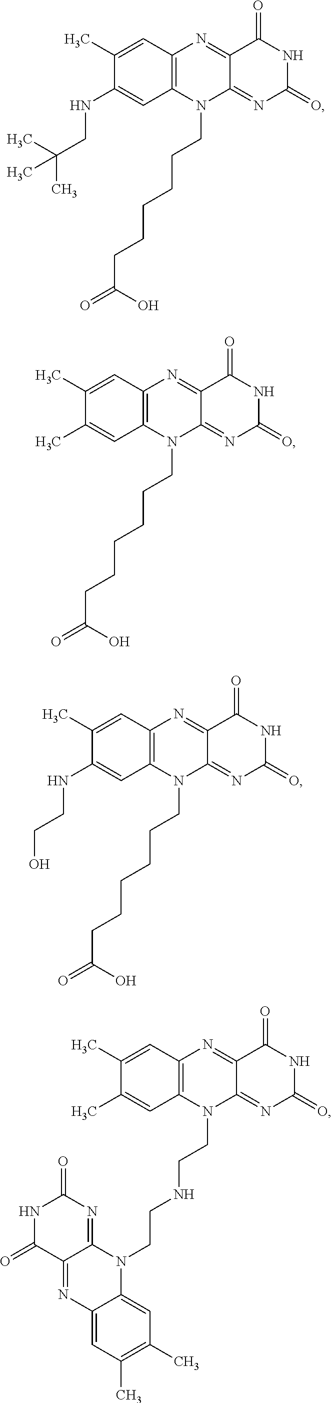

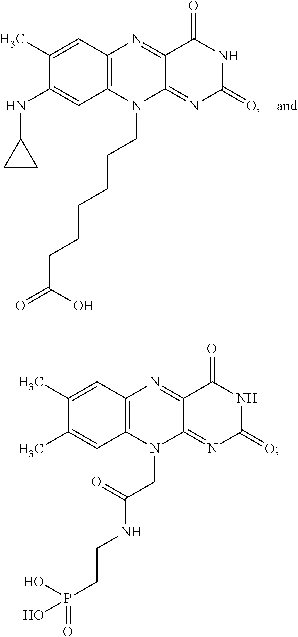

1.265. Formula I, or any of 1.1-1.264, wherein the Compound of Formula I binds to FMN riboswitch, e.g., with an IC₅₀ of less than or equal to 10 μM, preferably less than 1 μM, more preferably less than 100 nM, most preferably less than 10 nM in a binding assay, for example, as described in Example 1 and/or has a minimum inhibitory concentration of less than 128 μg/mL, preferably less than 32 μg/mL in an assay, for example, as described in Example 1A,
   in free, salt or prodrug form.

In a particular aspect, the invention relates to a Compound of formula III,

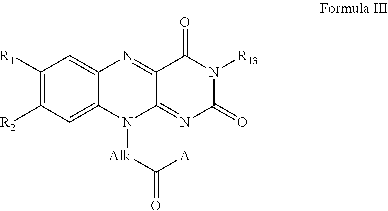

wherein:
(i) Alk is C₁₋₈ alkyl (e.g., n-butyl, n-pentyl, n-hexyl, 6,6-dimethylhexyl, n-heptyl);
(ii) A is —OR₉ or —N(R₁₄)(R₁₅);
(iii) R₉ is H, —C₁₋₈alkyl (e.g., methyl, ethyl, n-propyl, isopropyl, 1-methylpropyl, t-butyl, n-butyl, 1,1-dimethylpropyl, 2,2-dimethylpropyl, hex-5-ynyl), -haloC₁₋₈allyl (e.g., 2,2,2-trifluoroethyl), —C₁₋₄alkyl-OC(O)R₁₂, —C₁₋₄alkyl-O—C₁₋₄alkyl (e.g., —C(CH₃)(CH₃)OCH₃ or —C(CH₃)(CH₃)CH₂OCH₃), —C₁₋₄alkyl-C(O)-(morphylin-4-yl), —C₃₋₇cycloalkyl (e.g., cyclopentyl, cyclohexyl), C₃₋₇cycloalkyl-C₁₋₄alkyl (e.g., norbornan-2-yl-methyl) wherein the cycloalkyl is optionally substituted with hydroxy group; aryl (e.g., phenyl) or aryl-C₁₋₄alkyl (e.g, Benzyl, 1-methyl-2-phenylethyl), wherein said aryl is optionally substituted with one or more halo or C₁₋₄alkoxy (e.g., 3-chloro-phenylmethyl, 3-fluoro-phenylmethyl, 4-methoxy-3-fluorophenylmethyl, 2-methoxyphenyl, 2-(3,4-dihydroxyphenyl)ethyl);
(iv) R₁ is H, C₁₋₈ alkyl (e.g., methyl);
(v) R₂ is H, halo (e.g., chloro), —O—C₃₋₇cycloalkyl (e.g., —O-cyclopentyl), —O—C₀₋₇ alkylC₃₋₇cycloalkyl (e.g., —O-cyclopentyl, —O—CH₂-cyclopentyl), —N(R₄)(R₅), —(CH₂)—N(R₄)(R₅), —C₀₋₄alkyl-C₃₋₇cycloalkyl (e.g., cyclopropyl, cyclopentylmethyl), heteroC₃₋₇cycloalkyl (e.g., pyrrolidin-1-yl), 1-cyclopropyl-6-fluoro-7-[4-piperazin-1-yl]-4-oxo-quinoline-3-carboxylic acid), C₁₋₈alkyl (e.g., methyl or ethyl) or —O—C₁₋₈alkyl (e.g., methoxy), wherein the alkyl group is optionally substituted with one or more halo (e.g., fluoro) or hydroxy groups (e.g., trifluoromethyl, —O—CH₂CH₂OH);
(vi) R₄ and R₅ are independently
   a. H,
   b. —C₀₋₄alkyl-C₃₋₇cycloalkyl (e.g., cyclopropyl, cyclopentyl, cyclohexyl or cyclopentyl-methyl),
   c. heteroC₃₋₇cycloalkyl (e.g., pyrrolidinyl, e.g., pyrrolidin3-yl),
   d. aryl (e.g., phenyl or 2,2-dimethylpropyl),
   e. aryl-C₁₋₈alkyl wherein the aryl is optionally substituted with halo (e.g., 4-fluorophenylethyl),
   f. —(CH₂)₃—N(H)—(CH₂)₄—N(H)—(CH₂)₃—N(H)₂,
   g. —C₁₋₈alkyl (e.g., methyl) wherein said alkyl is optionally substituted with one or more hydroxy groups (e.g., 2,3-dihydroxypropyl, 2,3,4,5,6-pentahydroxyhexyl, hydroxyethyl);
(vii) R₁₂ is C₁₋₈alkyl (e.g., methyl, ethyl, t-Butyl) or —OC₁₋₈alkyl (e.g., methoxy, ethoxy, t-butoxy); and
(viii) R₁₃ is H or C₁₋₄alkyl (e.g., methyl);
(ix) R₁₄ and R₁₅ are independently H, —OH, —S(O)₂CH₃, —OBn or —C₁₋₄alkyl (e.g., methyl),
   in free, salt or prodrug form.

In another embodiment, the invention relates to the following formulae:
3.1 a compound of Formula III, wherein Alk is C₁₋₈ alkyl (e.g., n-butyl, n-pentyl, n-hexyl, 6,6-dimethylhexyl, n-heptyl);
3.2 a compound of Formula III or 3.1, wherein Alk is C₄₋₈ alkyl alkyl (e.g., n-butyl, n-pentyl, n-hexyl, 6,6-dimethylhexyl, n-heptyl
3.3 a compound of Formula III or 3.1, wherein Alk is n-hexyl or 6,6-dimethylhexyl;
3.4 a compound of Formula III, or any of 3.1-3.3, wherein A is —OR₉ or —N(R₁₄)(R₁₅);
3.5 a compound of Formula III, or any of 3.1-3.3, wherein A is —OR₉;
3.6 a compound of Formula III, or any of 3.1-3.5, wherein R₉ is H, —C₁₋₈alkyl (e.g., methyl, ethyl, n-propyl, isopropyl, 1-methylpropyl, t-butyl, n-butyl, 1,1-dimethylpropyl, 2,2-dimethylpropyl, hex-5-ynyl), -haloC₁₋₈alkyl (e.g., 2,2,2-trifluoroethyl), —C₁₋₄alkyl-OC(O)R₁₂, —C₁₋₄alkyl-O—C₁₋₄alkyl (e.g., —C(CH₃)(CH₃)OCH₃ or —C(CH₃)(CH₃)CH₂OCH₃), —C₁₋₄alkyl-C(O)-(morphylin-4-yl), —C₃₋₇cycloalkyl (e.g., cyclopentyl, cyclohexyl), C₃₋₇cycloalkyl-C₁₋₄alkyl (e.g., norbornan-2-yl-methyl) wherein the cycloalkyl is optionally substituted with hydroxy group; aryl (e.g., phenyl) or aryl-C₁₋₄alkyl (e.g, Benzyl, 1-methyl-2-phenylethyl), wherein said aryl is optionally substituted with one or more halo or C₁₋₄alkoxy (e.g., 3-chloro-phenylmethyl, 3-fluoro-phenylmethyl, 4-methoxy-3-fluorophenylmethyl, 2-methoxyphenyl, 2-(3,4-dihydroxyphenyl)ethyl);
3.7 a compound of Formula III, or any of 3.1-3.5, wherein R₉ is —C₁₋₈alkyl (e.g., methyl, ethyl, n-propyl, isopropyl, 1-methylpropyl, t-butyl, n-butyl, 1,1-dimethylpropyl, 2,2-dimethylpropyl, hex-5-ynyl);
3.8 a compound of Formula III, or any of 3.1-3.5, wherein R₉ is H,
3.9 a compound of Formula III, or any of 3.1-3.5, wherein R₉ is haloC₁₋₈alkyl (e.g., 2,2,2-trifluoroethyl), —C₁₋₄alkyl-OC(O)R₁₂, —C₁₋₄alkyl-O—C₁₋₄alkyl (e.g., —C(CH₃)(CH₃)OCH₃ or —C(CH₃)(CH₃)CH₂OCH₃), —C₁₋₄alkyl-C(O)-(morphylin-4-yl), —C₃₋₇cycloalkyl (e.g., cyclopentyl, cyclohexyl), C₃₋₇cycloalkyl-C₁₋₄alkyl (e.g., norbornan-2-yl-methyl) wherein the cycloalkyl is optionally substituted with hydroxy group; aryl (e.g., phenyl) or aryl-C₁₋₄alkyl (e.g, Benzyl, 1-methyl-2-phenylethyl), wherein said aryl is optionally substituted with one or more halo or C₁₋₄alkoxy (e.g., 3-chloro-phenylmethyl, 3-fluoro-phenylmethyl, 4-methoxy-3-fluorophenylmethyl, 2-methoxyphenyl, 2-(3,4-dihydroxyphenyl)ethyl);
3.10 a compound of Formula III, or any of 3.1-3.3, wherein A is —N(R₁₄)(R₁₅);
3.11 a compound of Formula 3.10, wherein R₁₄ and R₁₅ are independently H, —OH, —S(O)₂CH₃, —OBn or —C₁₋₄alkyl (e.g., methyl);
3.12 a compound of Formula III or any of 3.1-3.11, wherein R₁ is H, C₁₋₈ alkyl (e.g., methyl);
3.13 a compound of Formula III or any of 3.1-3.12, wherein R₂ is H, halo (e.g., chloro), —O—C₃₋₇cycloalkyl (e.g., —O-cyclopentyl), —O—C₀₋₇ alkylC₃₋₇cycloalkyl (e.g., —O-cyclopentyl, —O—CH₂-cyclopentyl), —N(R₄)(R₅), —(CH₂)—N(R₄)(R₅), —C₀₋₄-alkyl-C₃₋₇cycloalkyl (e.g., cyclopropyl, cyclopentylmethyl), heteroC₃₋₇cycloalkyl (e.g., pyrrolidin-1-yl), 1-cyclopropyl-6-fluoro-7-[4-piperazin-1-yl]-4-oxo-quinoline-3-carboxylic acid), C₁₋₈alkyl (e.g., methyl or ethyl) or —O—C₁₋₈alkyl (e.g., methoxy), wherein the alkyl group is optionally substituted with one or more halo (e.g., fluoro) or hydroxy groups (e.g., trifluoromethyl, —O—CH₂CH₂OH);
3.14 a compound of Formula III or any of 3.1-3.12, wherein R₂ is —N(R₄)(R₅);
3.15 a compound of Formula 3.14, wherein R₄ and R₅ are independently H, —C₀₋₄-alkyl-C₃₋₇cycloalkyl (e.g., cyclopropyl, cyclopentyl, cyclohexyl or cyclopentyl-methyl), heteroC₃₋₇cycloalkyl (e.g., pyrrolidinyl, e.g., pyrrolidin3-yl), aryl (e.g., phenyl or 2,2-dimethylpropyl), aryl-C₁₋₈alkyl wherein the aryl is optionally substituted with halo (e.g., 4-fluorophenylethyl), —(CH₂)₃—N(H)—(CH₂)₄—N(H)—(CH₂)₃—N(H)₂, —C₁₋₈alkyl (e.g., methyl) wherein said alkyl is optionally substituted with one or more hydroxy groups (e.g., 2,3-dihydroxypropyl, 2,3,4,5,6-pentahydroxyhexyl, hydroxyethyl);
3.16 a compound of Formula 3.14, wherein R₄ is H and R₅ is aryl (e.g., phenyl);
3.17 a compound of Formula 3.14, wherein R₄ and R₅ are —C₁₋₈alkyl (e.g., methyl);
3.18 a compound of Formula III or any of 3.1-3.17, wherein R₁₂ is C₁₋₈alkyl (e.g., methyl, ethyl, t-Butyl) or —OC₁₋₈alkyl (e.g., methoxy, ethoxy, t-butoxy);
3.19 a compound of Formula III or any of 3.1-3.18, wherein R₁₃ is H or C₁₋₄alkyl (e.g., methyl)
3.20 a compound of Formula III or any of 3.1-3.19 wherein said compound is selected from the following:

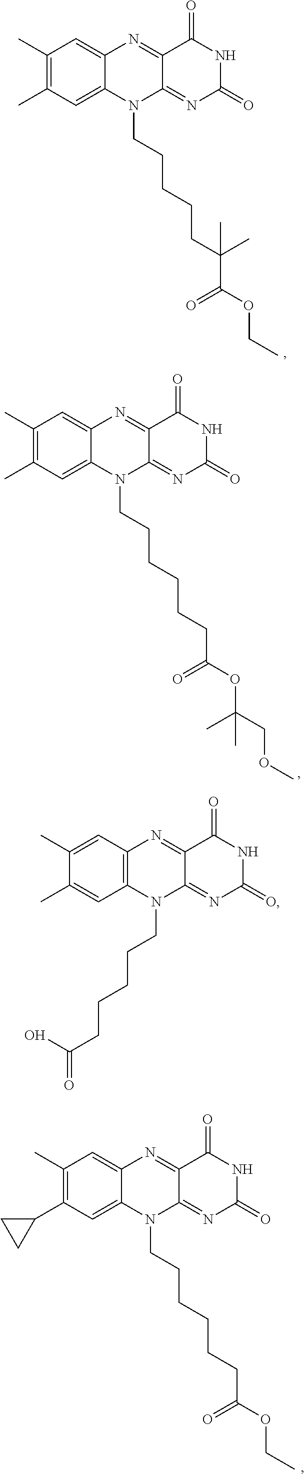

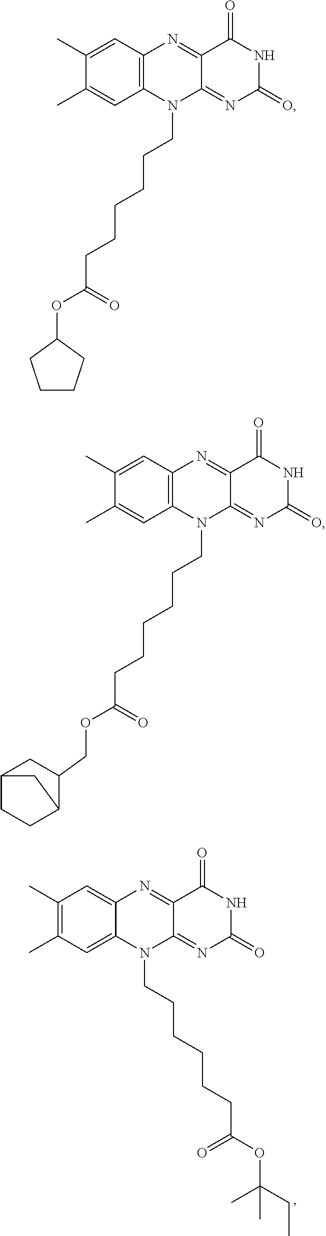

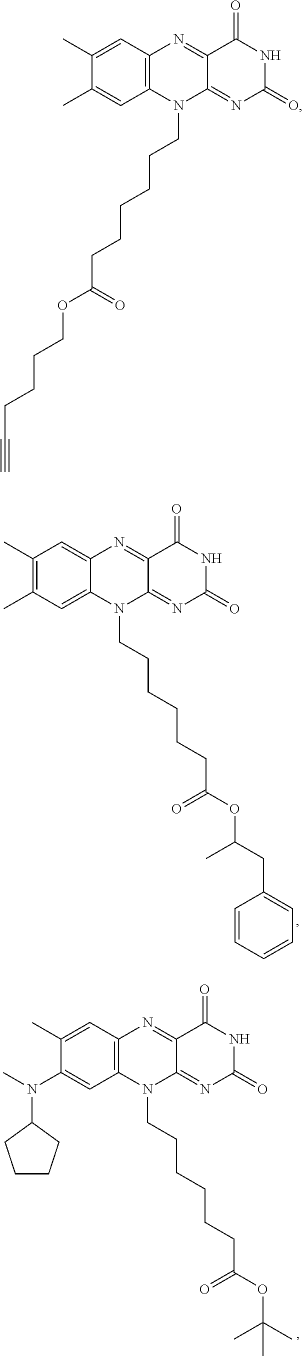

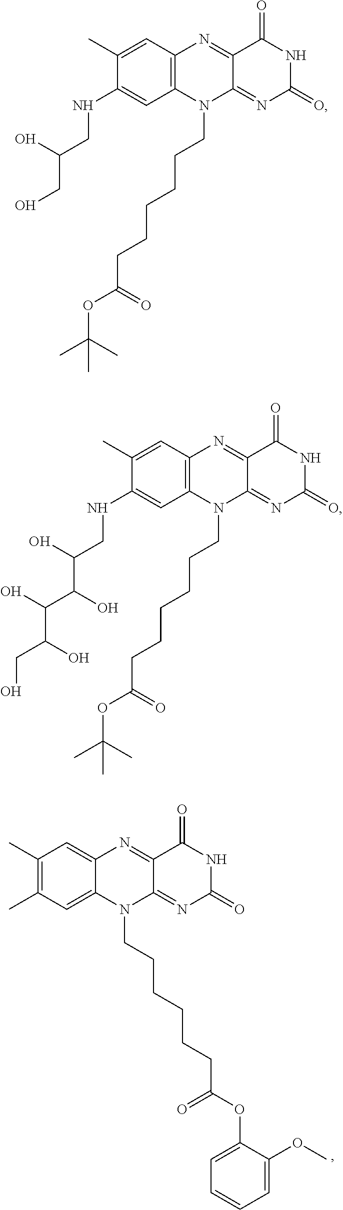

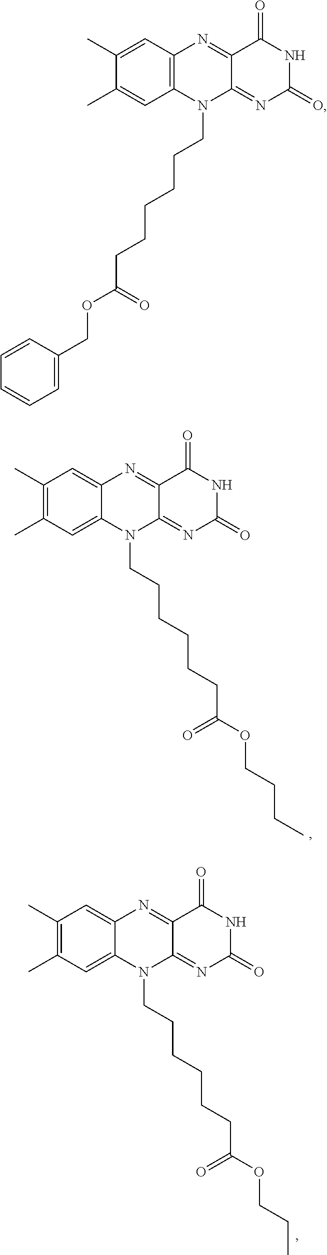

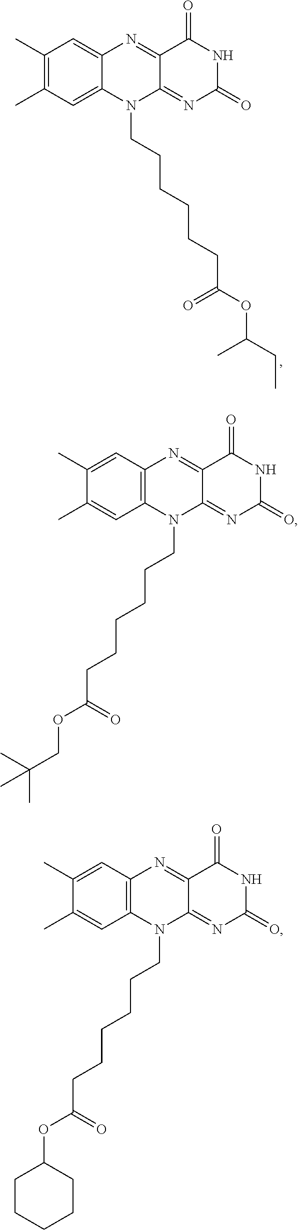

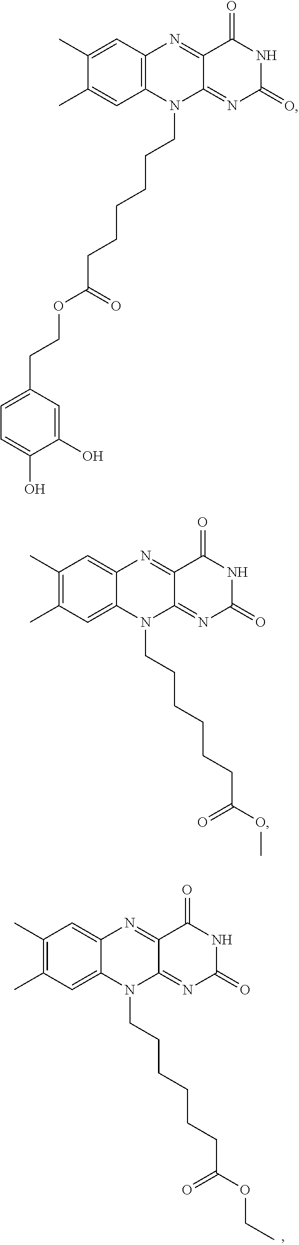

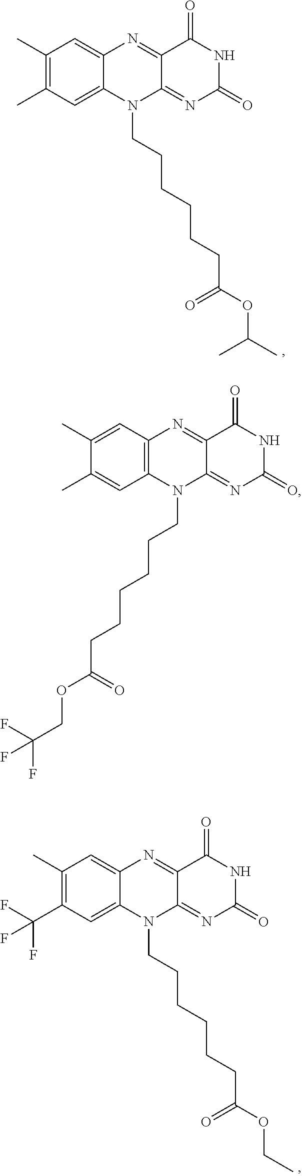

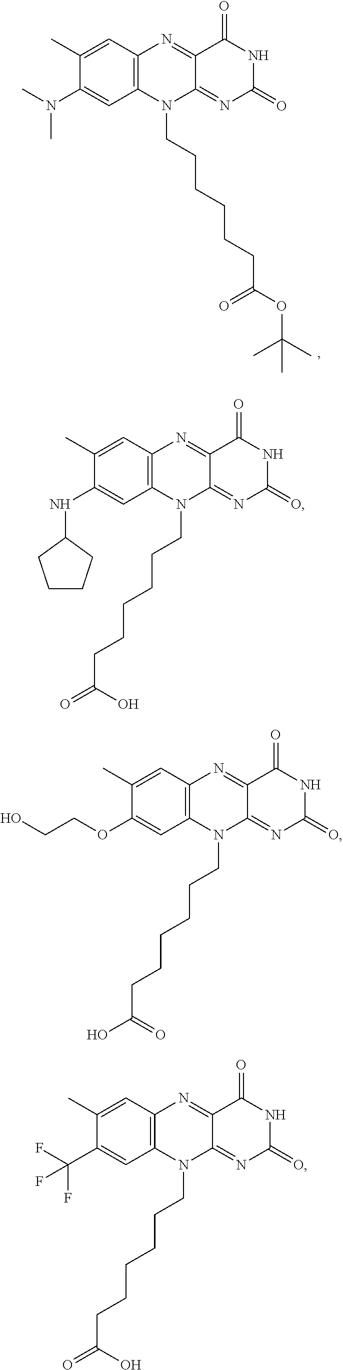

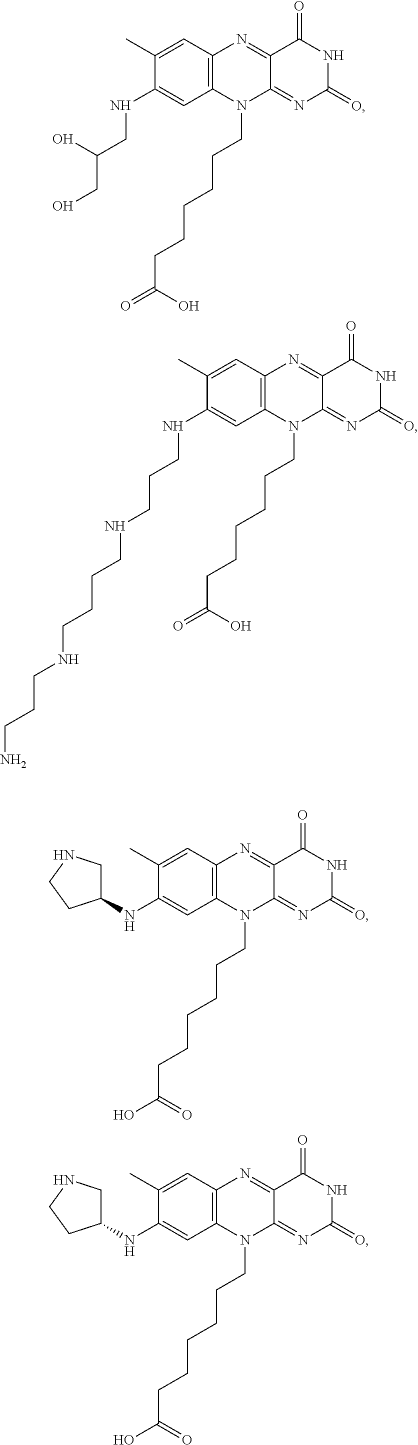

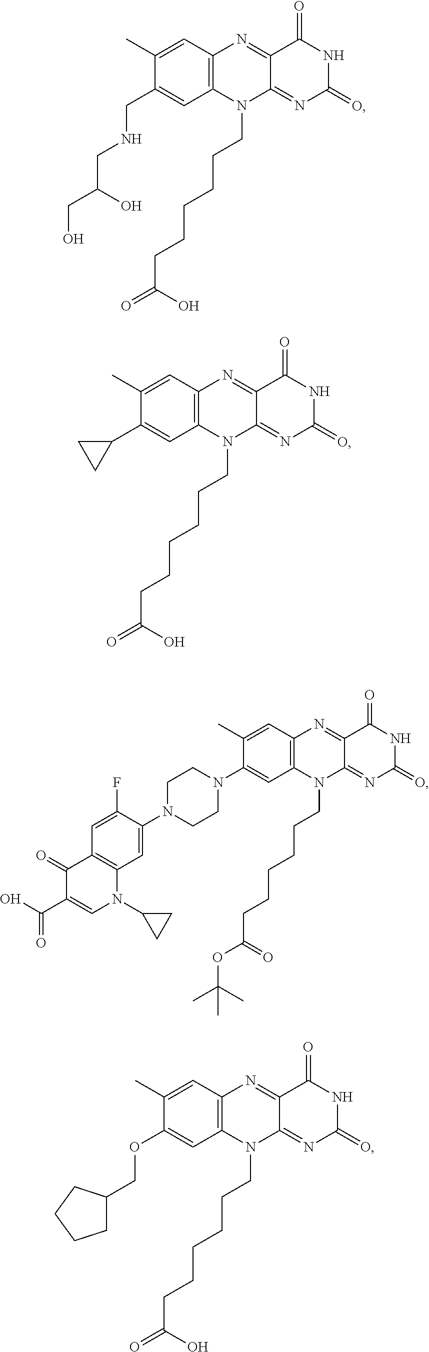

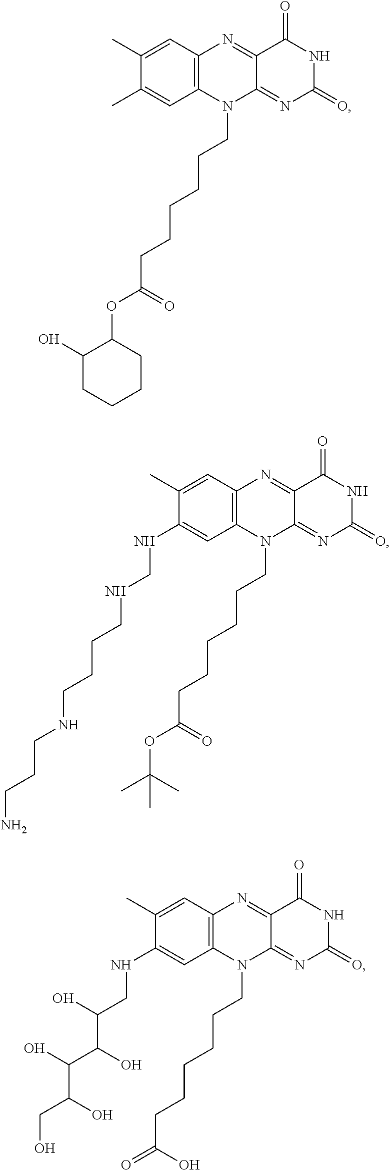

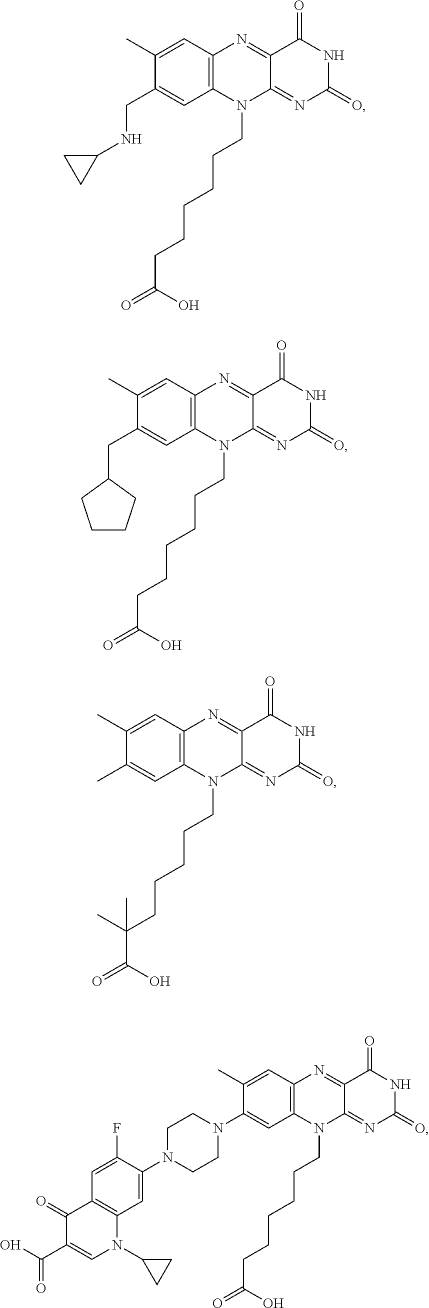

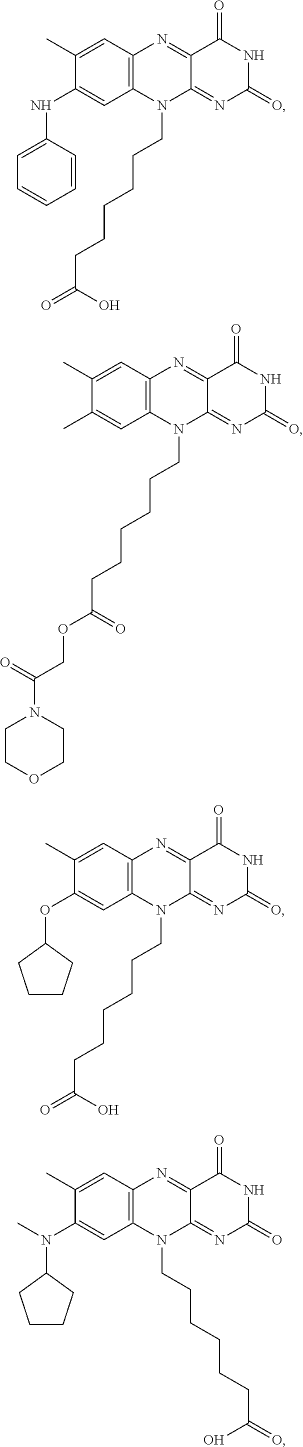

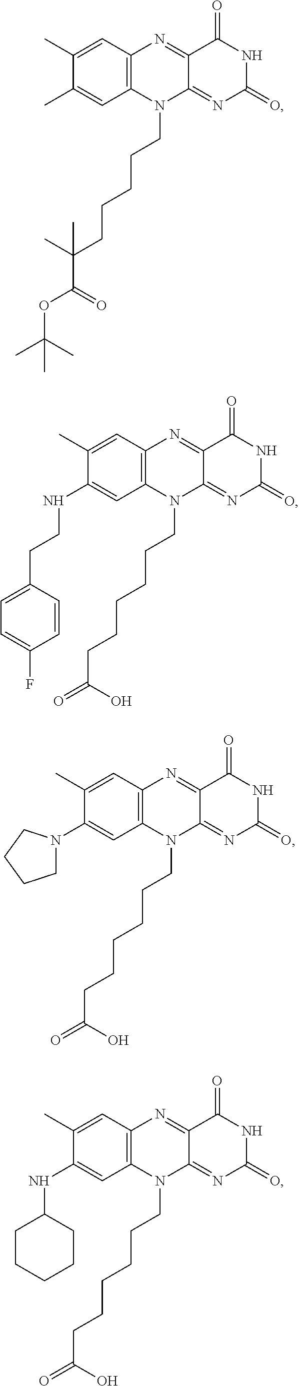

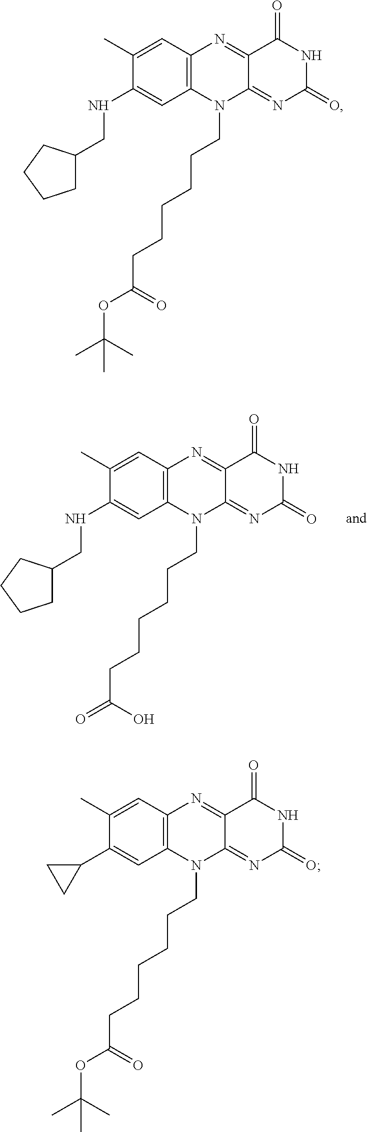

3.21 a compound of Formula III or any of 3.1-3.19 wherein said compound is selected from any one of the following:

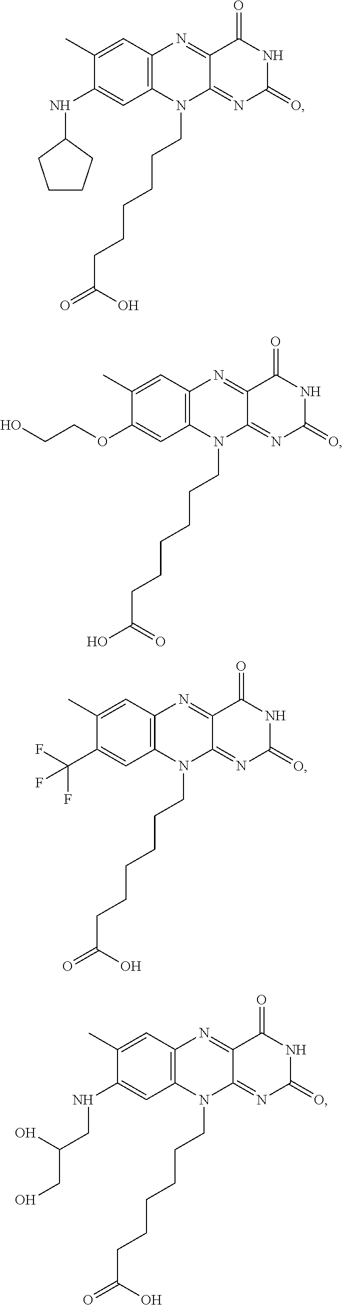

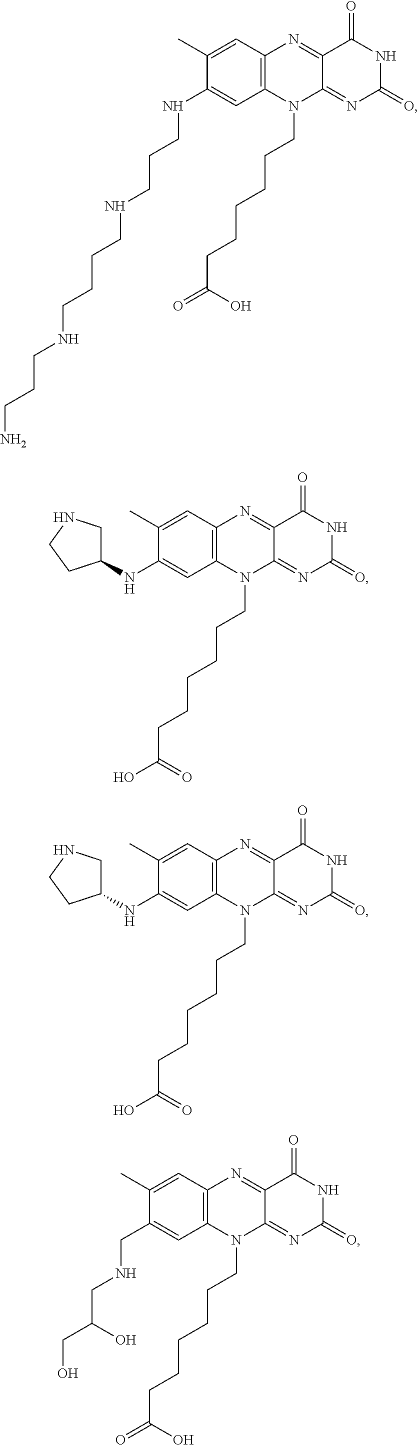

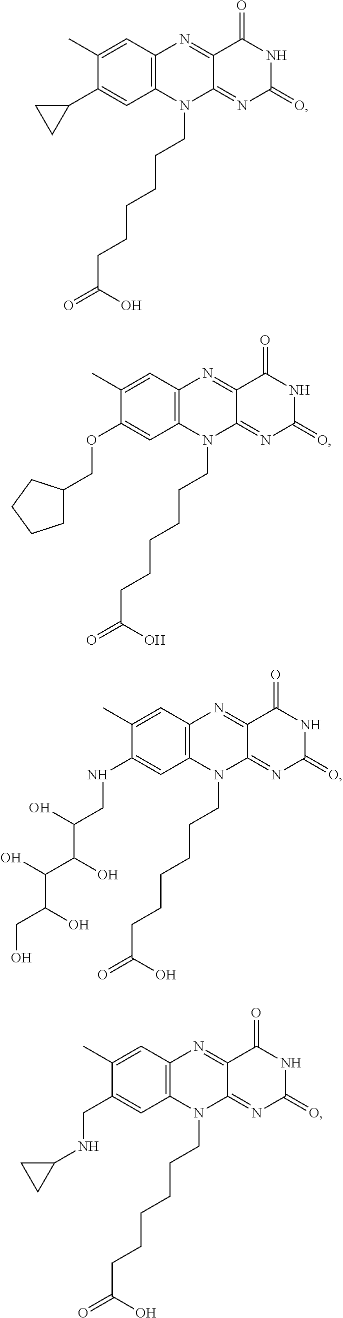

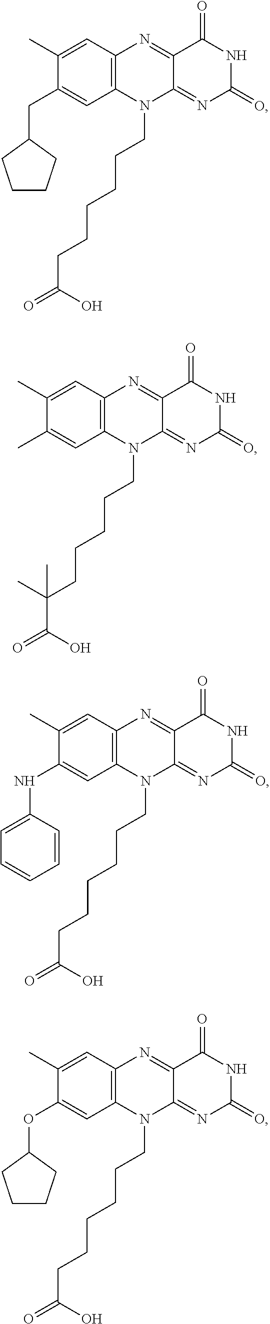

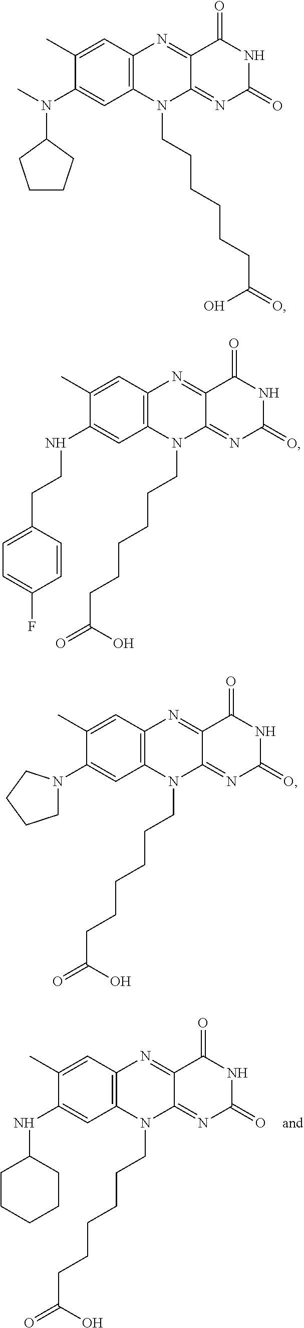

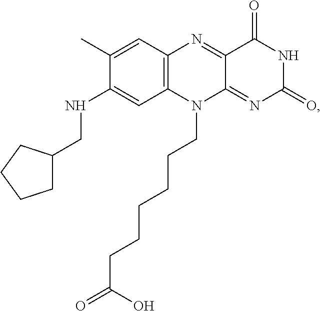

3.22 a compound of Formula III or any of 3.1-3.19 wherein said compound is selected from anyone of the following:

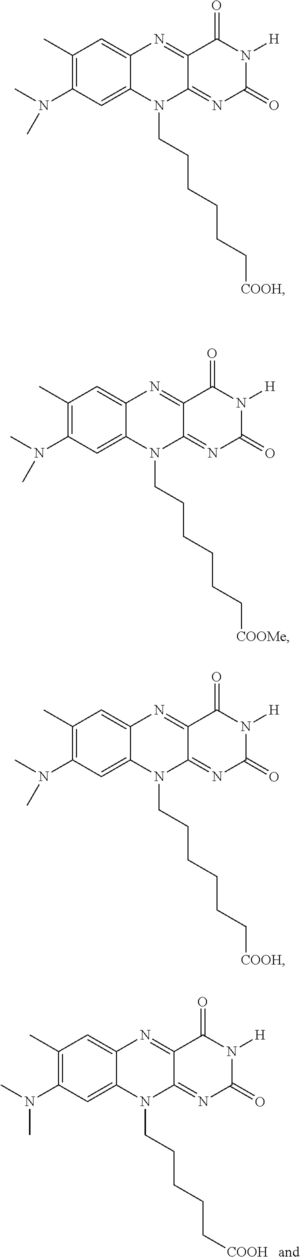

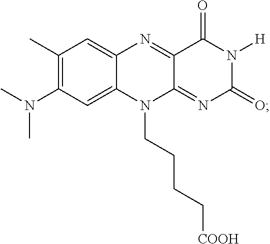

3.23 a compound of Formula III or any of 3.1-3.19 wherein said compound is selected from the following:

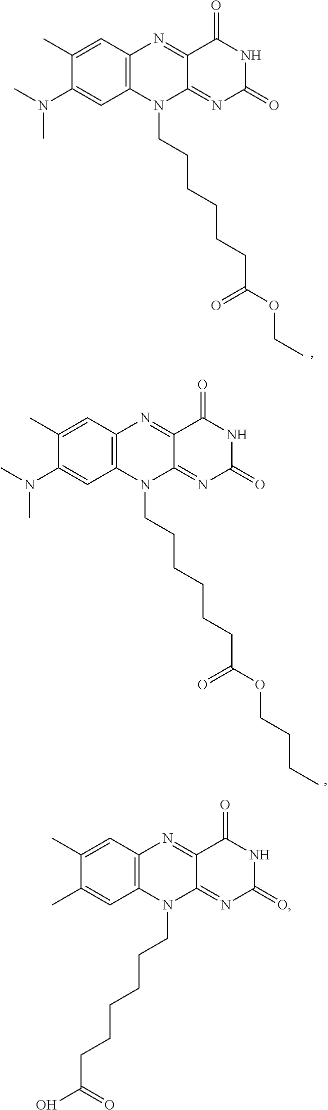

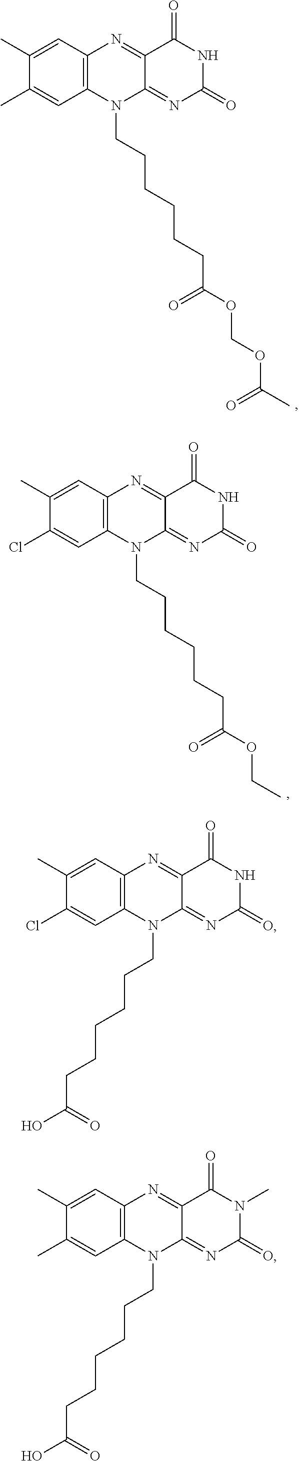

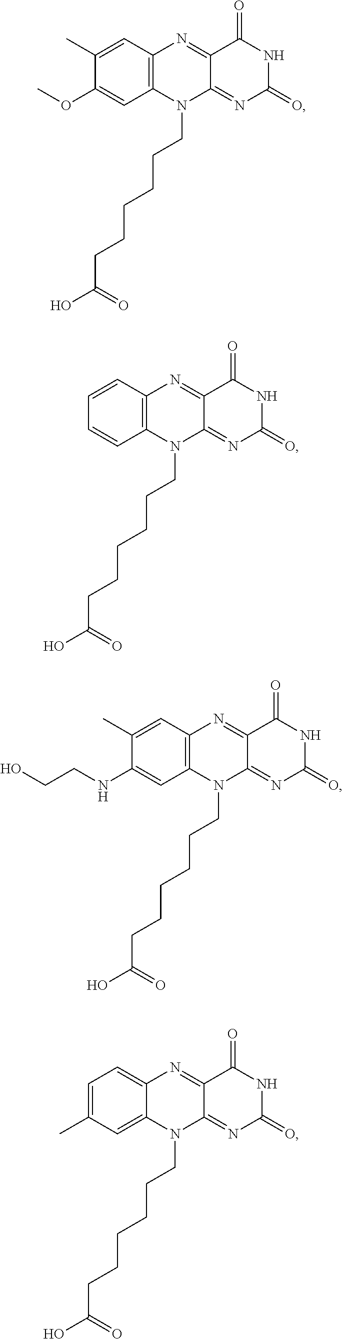

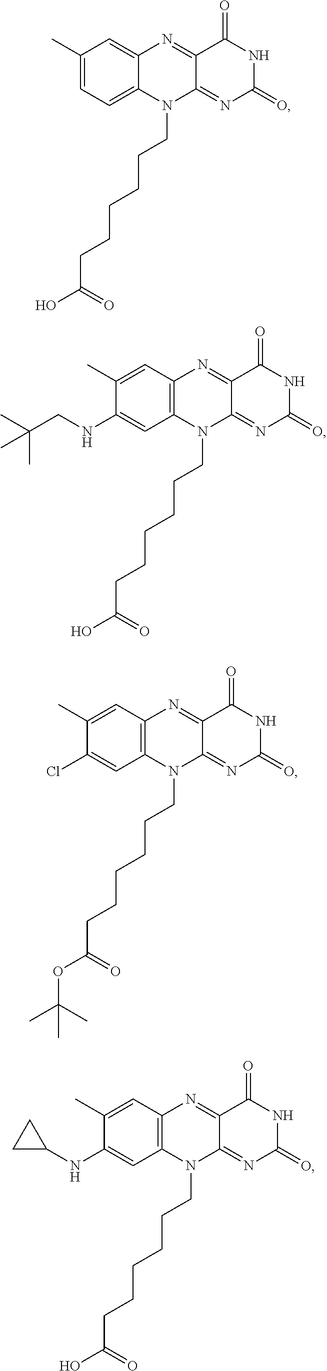

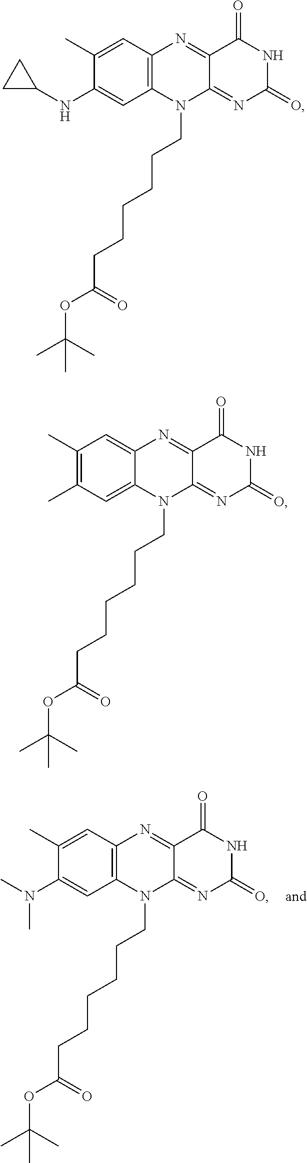

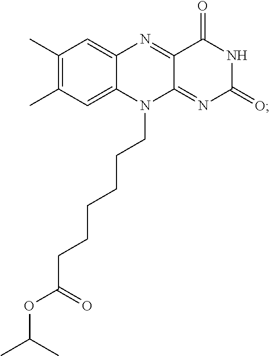

3.24 a compound of Formula III or any of 3.1-3.19 wherein said compound is selected from the following:

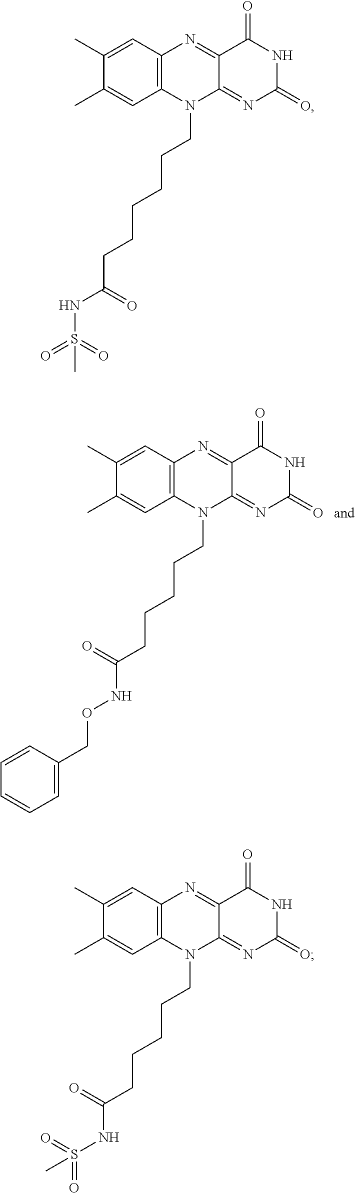

3.25 a compound of Formula III or any of 3.1-3.19 wherein said compound is:

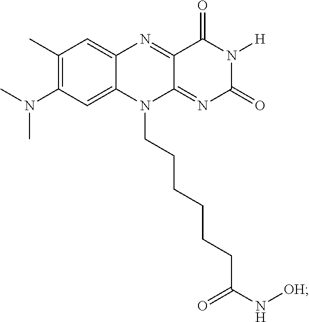

3.26 a compound of Formula III or any of 3.1-3.19 wherein said compound is:

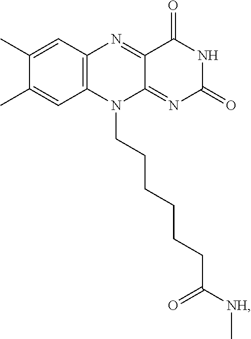

3.27 any of the preceding formulae wherein the Compound (a) binds to FMN riboswitch, e.g., with an IC₅₀ of less than or equal to 10 μM, preferably less than 1 μM, more preferably less than 100 nM, most preferably less than 10 nM in a binding assay, for example, as described in Example 1 and/or (b) has a minimum inhibitory concentration (MIC) of less than 128 μg/mL, preferably less than 32 μg/mL, in an assay, for example, as described in Example 1A;
   in free, salt or prodrug form.

In a particular aspect, the invention relates to a Compound of formula IV,

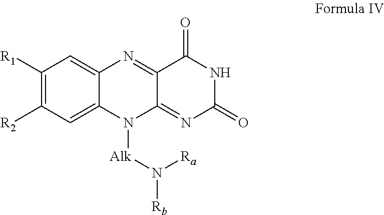

wherein:
(i) Alk is C₁₋₈ alkyl (e.g., ethyl or n-butyl);
(ii) Rₐ and R_{b} are independently H, —C₁₋₄alkyl (e.g., methyl), —(CH₂)₃C(NH₂)(COOH)CHF₂, —(CH₂)₃N(H)C(═NH)NH₂, —(CH₂)₅NH₂, —(CH₂)₂C(H)(OH)COOH, —C(O)(CH₂)₂COOH, —C₁₋₄alkyl-C(O)OR₉ (e.g., —CH₂CH₂CH₂CH₂C(O)OR₉, —CH₂CH₂CH₂C(O)OR₉, —CH₂CH₂C(O)OR₉ or —CH₂C(O)OR₉, —C(CH₃)(CH₃)C(O)OR₉), —C(O)CH₃, aryl (e.g., phenyl), —C(O)-aryl, aryl-C₁₋₈alkyl (e.g., benzyl, naphtha-1-ylmethyl, naphth-2-ylmethyl, phenylethyl, phenylpropyl, naphtha-1-ylethyl), heteroaryl, heteroaryl-C₁₋₄alkyl (e.g., pyrid-2-ylmethyl, pyrid-3-ylmethyl or quinoxalinyl), wherein said aryl and heteroaryl groups are optionally substituted with one or more groups selected from —C(O)OR₉, —S(O)₂NH₂, —CH₂NH₂, halo (e.g., chloro), C₁₋₄alkoxy (e.g., methoxy), C₁₋₄alkyl (e.g., methyl);
(iii) R₁ is H, C₁₋₈ alkyl (e.g., methyl);
(iv) R₂ is H, halo (e.g., chloro), —O—C₃₋₇cycloalkyl (e.g., —O-cyclopentyl), —N(R₄)(R₅), C₃₋₇cycloalkyl (e.g., cyclopropyl), C₁₋₈alkyl (e.g., methyl or ethyl) or —O—C₁₋₈alkyl wherein the alkyl group is optionally substituted with one or more halo or hydroxyl groups (e.g., trifluoromethyl, —O—CH₂CH₂OH);
(v) R₄ and R₅ are independently H, C₃₋₇cycloalkyl (e.g., cyclopropyl or cyclopentyl), C₁₋₈alkyl (e.g., methyl) wherein said alkyl is optionally substituted with one or more hydroxy groups (e.g., 2,3-dihydroxypropyl, 2,3,4,5,6-pentahydroxyhexyl);
(vi) R₉ is H or C₁₋₄alkyl (e.g., t-butyl, isopropyl, methyl);
(vii) R₁₂ is C₁₋₈alkyl (e.g., methyl, ethyl, t-Butyl) or —OC₁₋₈alkyl (e.g., methoxy, ethoxy, t-butoxy), in free, salt or prodrug form.

In another embodiment, the invention relates to the following formulae:
4.1 a compound of Formula IV, wherein Alk is C₁₋₈ alkyl (e.g., ethyl or n-butyl);
4.2 a compound of Formula IV, wherein Alk is ethyl;
4.3 a compound of Formula IV, wherein Alk is n-butyl;
4.4 a compound of Formula IV, 4.1, 4.2 or 4.3 is wherein Rₐ and R_{b} are independently H, —C₁₋₄alkyl (e.g., methyl), —(CH₂)₃C(NH₂)(COOH)CHF₂, —(CH₂)₃N(H)C(═NH)NH₂, —(CH₂)₅NH₂, —(CH₂)₂C(H)(OH)COOH, —C(O)(CH₂)₂COOH, —C₁₋₄alkyl-C(O)OR₉ (e.g., —CH₂CH₂CH₂CH₂C(O)OR₉, —CH₂CH₂CH₂C(O)OR₉, —CH₂CH₂C(O)OR₉, —CH₂C(O)OR₉ or —C(CH₃)(CH₃)C(O)OR₉), —C(O)CH₃, aryl (e.g., phenyl), —C(O)-aryl, aryl-C₁₋₄alkyl (e.g., benzyl, naphtha-1-ylmethyl, naphth-2-ylmethyl, phenylethyl, phenylpropyl, naphtha-1-ylethyl), heteroaryl, heteroaryl-C₁₋₄alkyl (e.g., pyrid-2-ylmethyl, pyrid-3-ylmethyl or quinoxalinyl), wherein said aryl and heteroaryl groups are optionally substituted with one or more groups selected from —C(O)OR₉, —NH₂, —S(O)₂NH₂, —CH₂NH₂, halo (e.g., chloro), C₁₋₄alkoxy (e.g., methoxy), C₁₋₄alkyl (e.g., methyl);
4.5 a compound of Formula IV, 4.1, 4.2 or 4.3, wherein Rₐ is H and R_{b} is aryl (e.g., phenyl), aryl-C₁₋₄alkyl (e.g., benzyl, naphtha-1-ylmethyl, naphth-2-ylmethyl, phenylethyl, phenylpropyl, naphtha-1-ylethyl), heteroaryl, heteroaryl-C₁₋₄alkyl (e.g., pyrid-2-ylmethyl, pyrid-3-ylmethyl or quinoxalinyl), wherein said aryl and heteroaryl groups are optionally substituted with one or more groups selected from —C(O)OR₉, —NH₂, —S(O)₂NH₂, —CH₂NH₂, halo (e.g., chloro), C₁₋₄alkoxy (e.g., methoxy), C₁₋₄alkyl (e.g., methyl);
4.6 a compound of Formula IV, or any of 4.1-4.5, wherein R₉ is H or C₁₋₄alkyl (e.g., t-butyl, isopropyl, methyl);
4.7 a compound of Formula IV, or any of 4.1-4.5, wherein R₉ is H,
4.8 a compound of Formula IV, or any of 4.1-4.5, wherein R₉ is C₁₋₄alkyl (e.g., t-butyl, isopropyl, methyl);
4.9 a compound of Formula IV or any of 4.1-4.8 wherein said compound is selected from any one of the following:

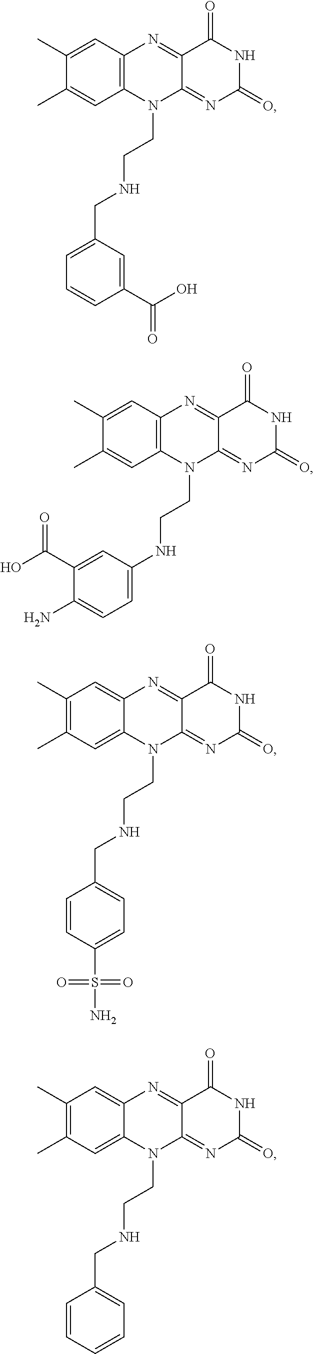

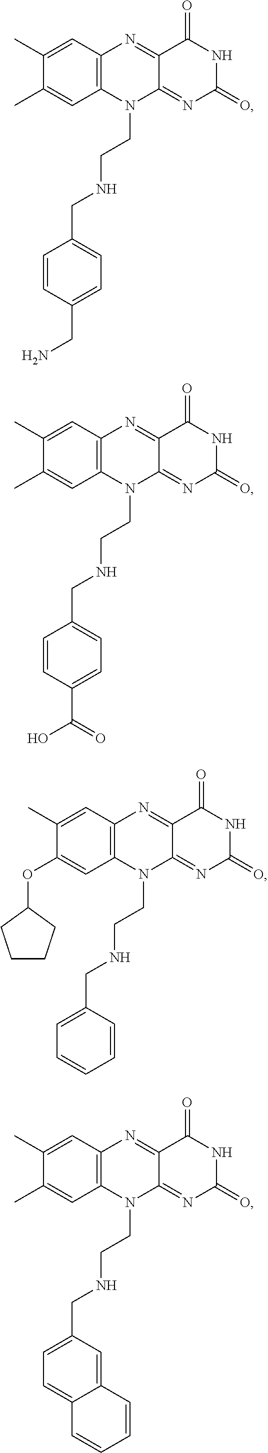

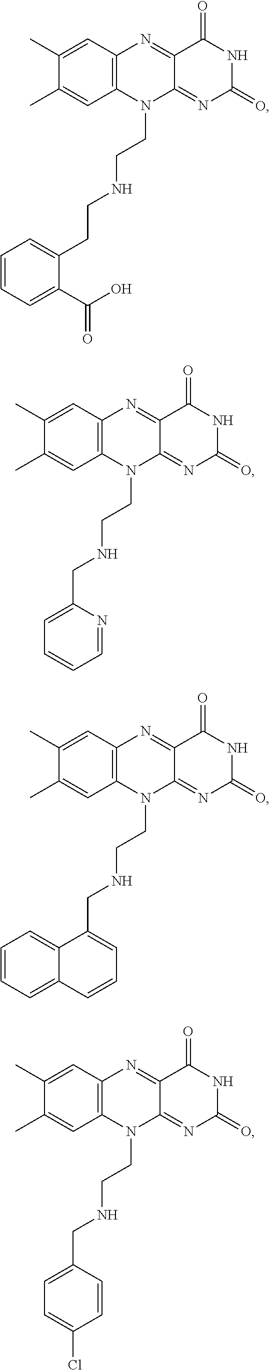

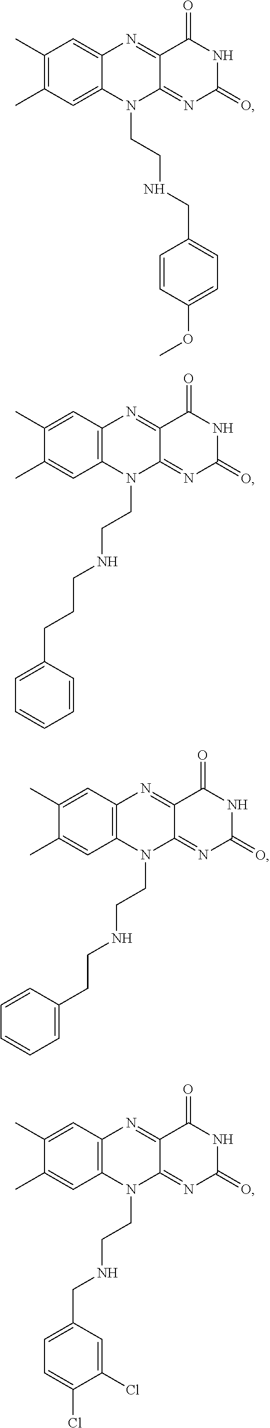

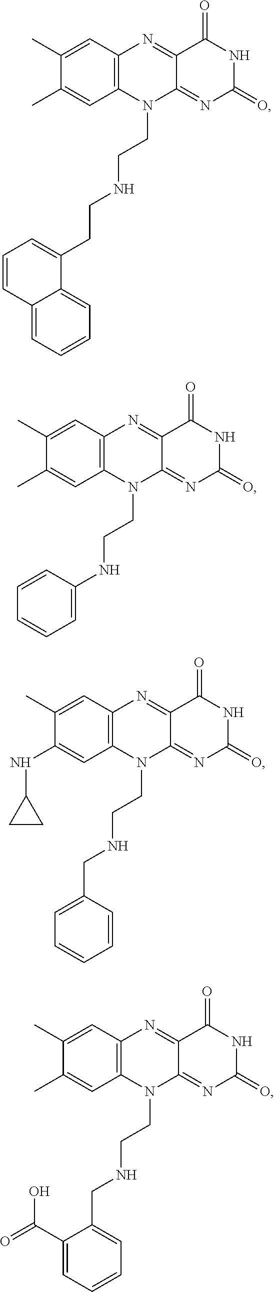

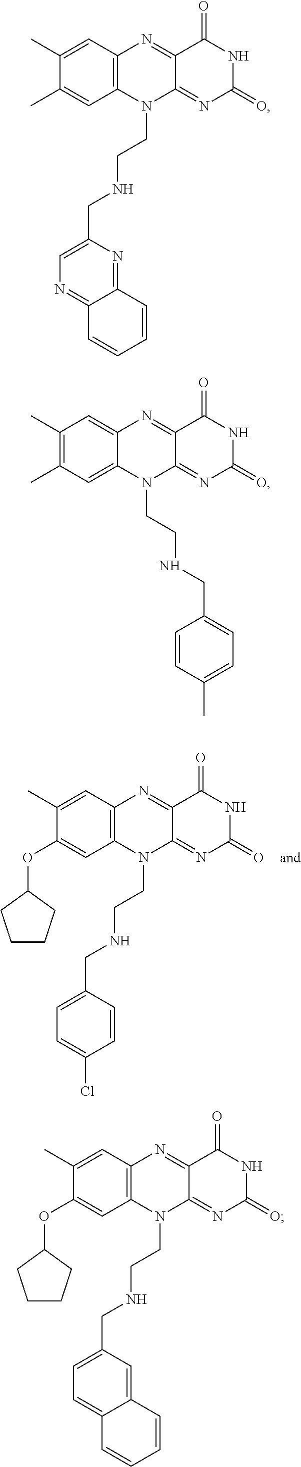

4.10 a compound of Formula IV or any of 4.1-4.6 wherein said compound is selected from any one of the following:

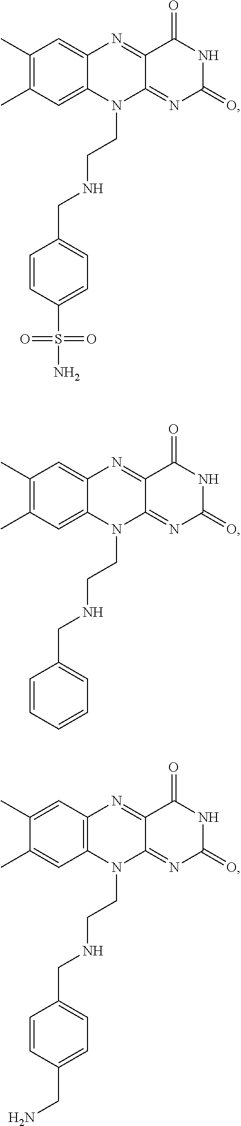

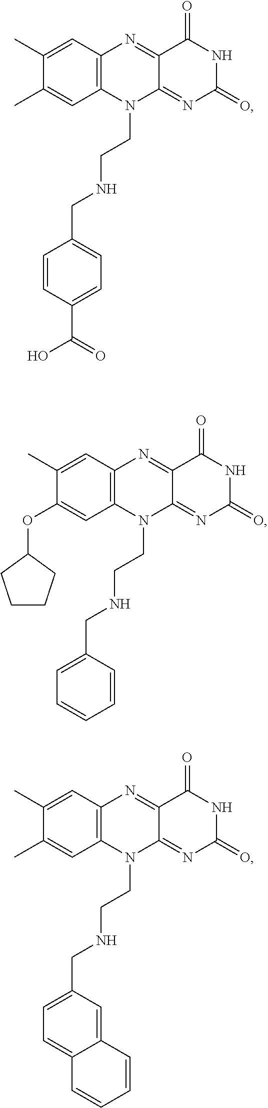

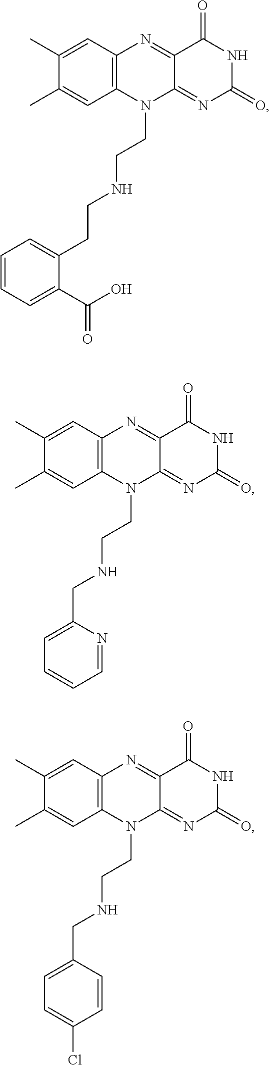

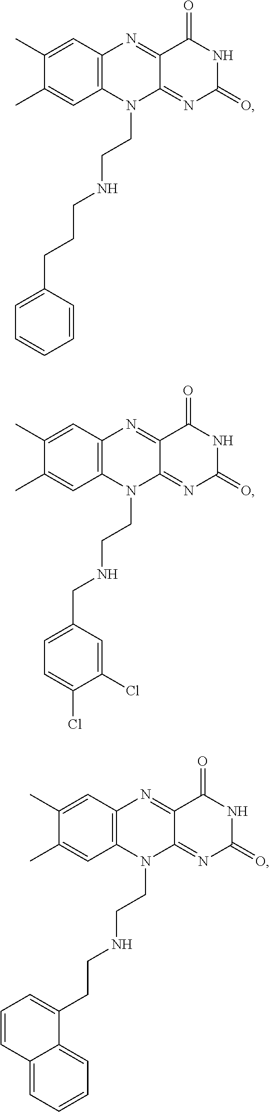

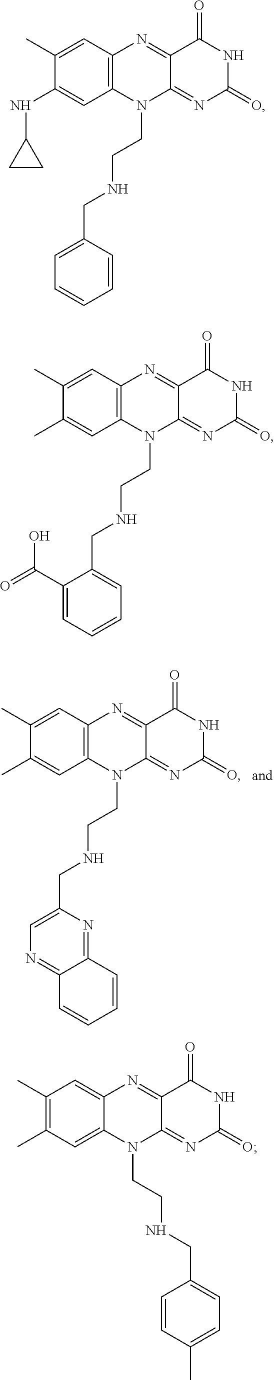

4.11 a compound of Formula IV or any of 4.1-4.6 wherein said compound is selected from any one of the following:

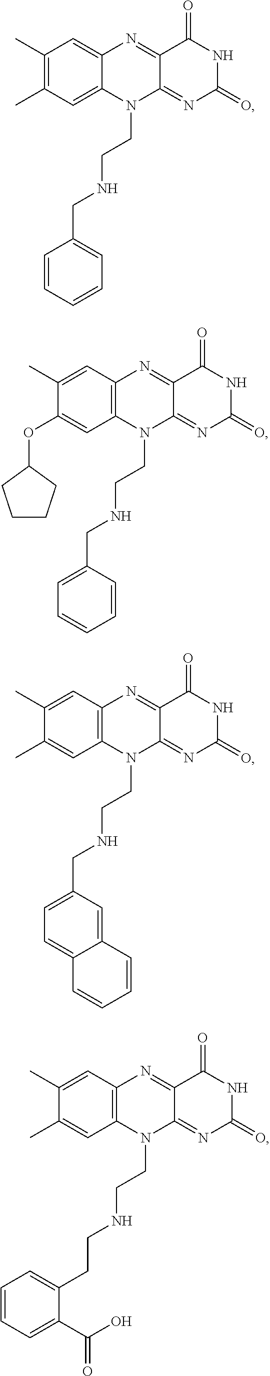

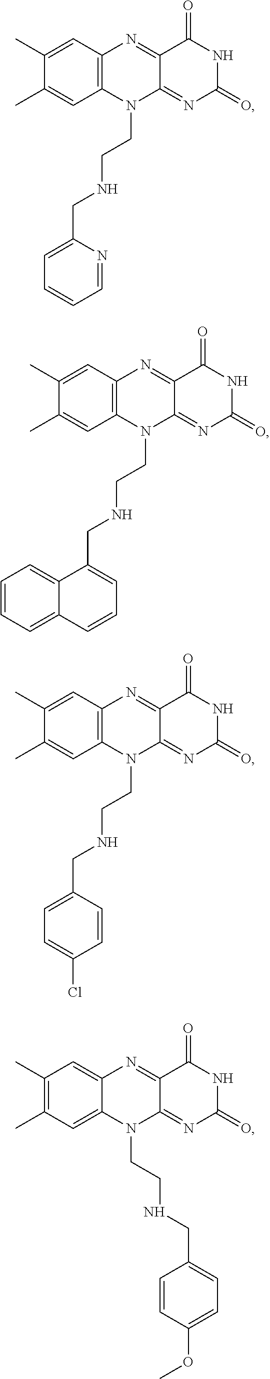

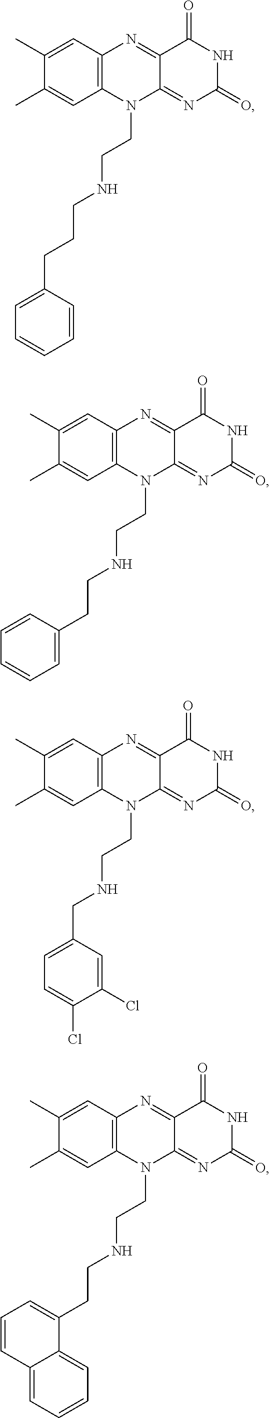

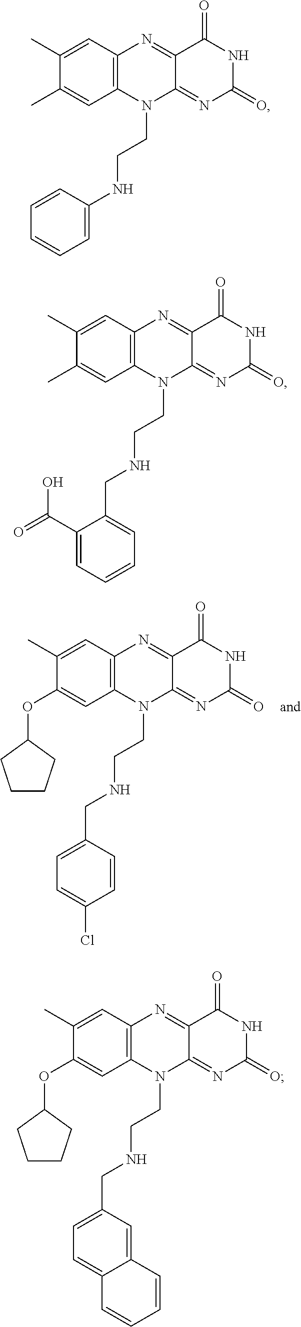

4.12 a compound of Formula IV or any of 4.1-4.6 wherein said compound is selected from any one of the following:

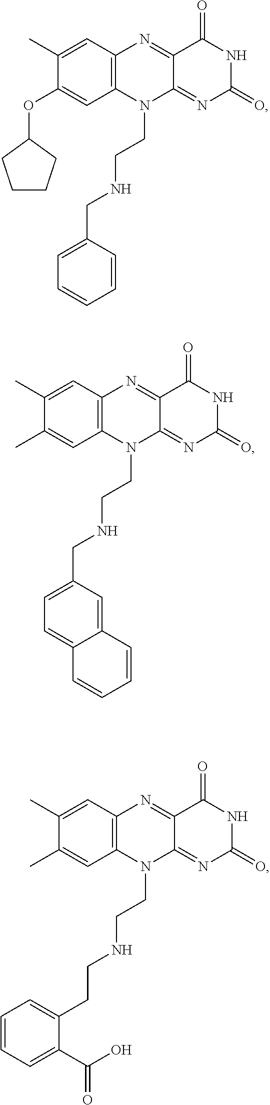

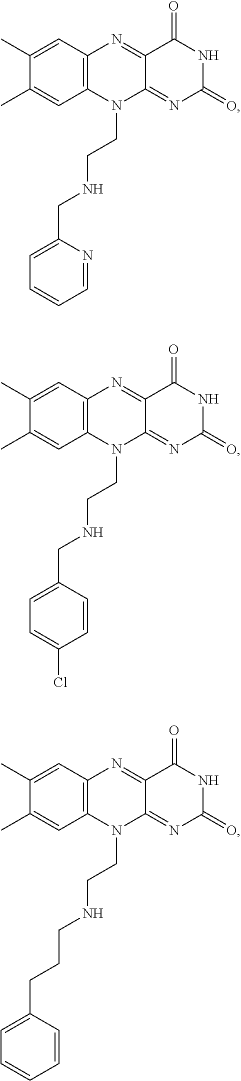

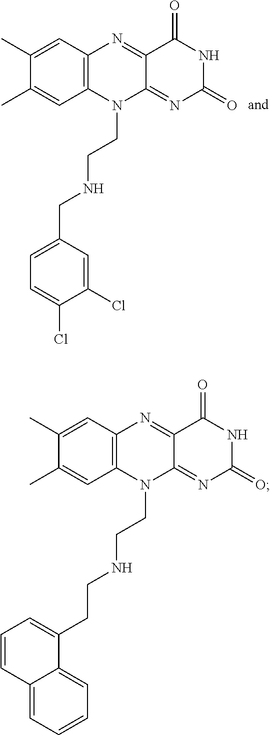

4.13 a compound of Formula IV or any of 4.1-4.6 wherein said compound is selected from any one of the following:

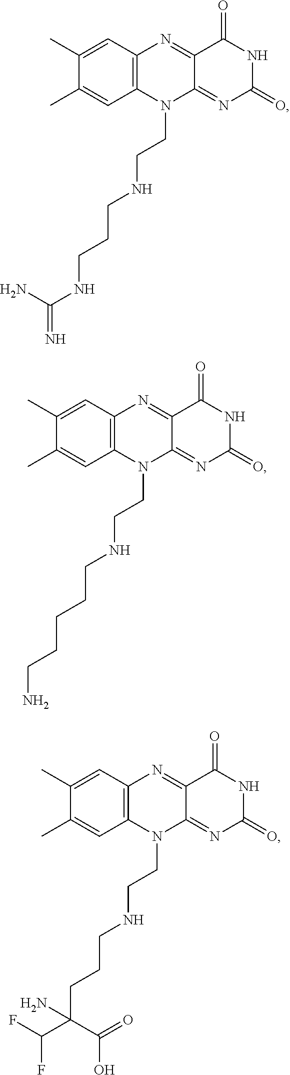

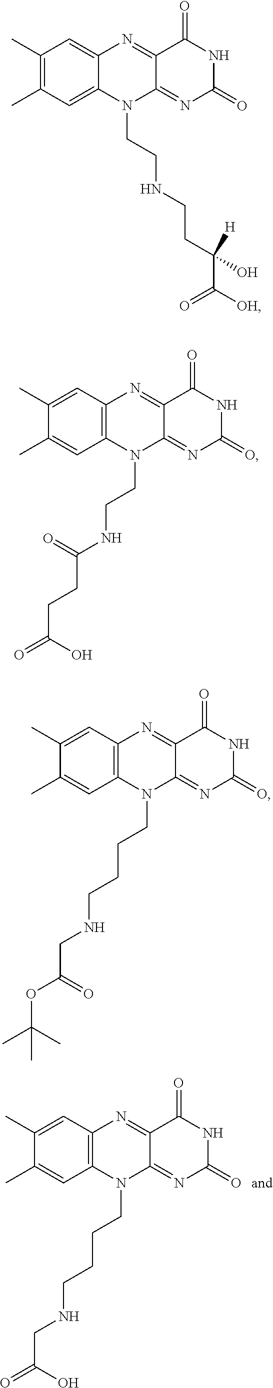

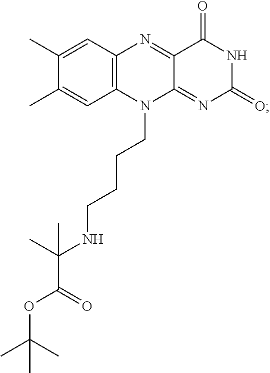

4.14 a compound of Formula IV or any of 4.1-4.6 wherein said compound is:

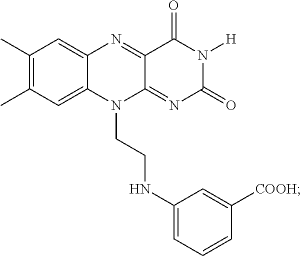

4.15 a compound of Formula IV or any of 4.1-4.6 wherein said compound is selected from:

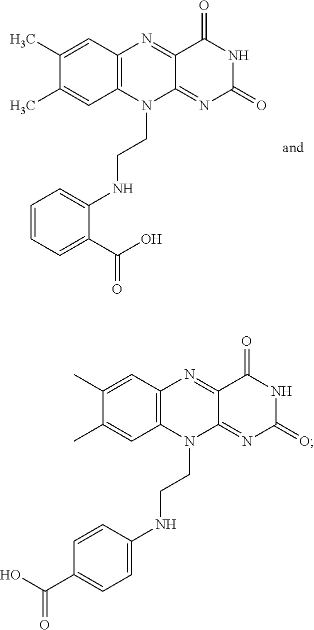

4.16 a compound of Formula IV, 4.1, 4.2 or 4.3, wherein Rₐ is —C₁₋₄alkyl (e.g., methyl) or —C₁₋₄alkyl-C(O)OR₉(e.g., —CH₂CH₂CH₂C(O)OR₉) and R_{b} is aryl (e.g., phenyl), aryl-C₁₋₄alkyl (e.g., benzyl, naphtha-1-ylmethyl, naphth-2-ylmethyl, phenylethyl, phenylpropyl, naphtha-1-ylethyl), heteroaryl, heteroaryl-C₁₋₄alkyl (e.g., pyrid-2-ylmethyl), wherein said aryl and heteroaryl groups are optionally substituted with one or more groups selected from —C(O)OR₉, —NH₂, —S(O)₂NH₂, —CH₂NH₂, halo (e.g., chloro), C₁₋₄alkoxy (e.g., methoxy), C₁₋₄alkyl (e.g., methyl);
4.17 a compound of Formula IV or any of 4.1, 4.2, 4.3, 4.12-4.16, wherein R₉ is H or C₁₋₄alkyl (e.g., t-butyl, isopropyl, methyl);
4.18 compound of Formula IV or any of 4.1, 4.2, 4.3, 4.12-4.16, wherein R₉ is H,
4.19 compound of Formula IV or any of 4.1, 4.2, 4.3, 4.12-4.16, wherein R₉ is C₁₋₄alkyl (e.g., t-butyl, isopropyl, methyl);
4.20 a compound of Formula IV or any of 4.1, 4.2, 4.3, 4.12-4.19, wherein said compound is selected from any one of the following:

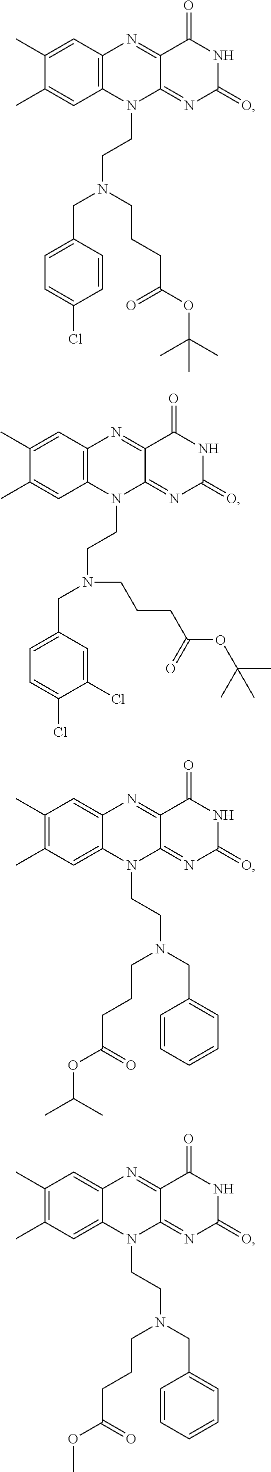

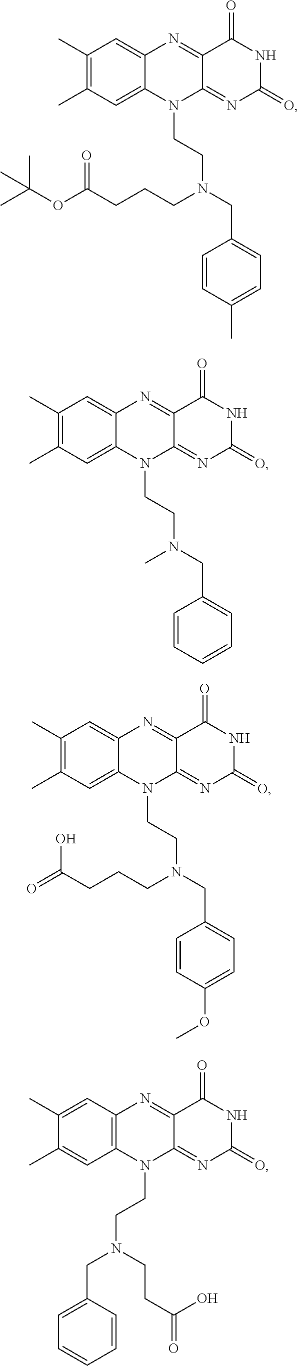

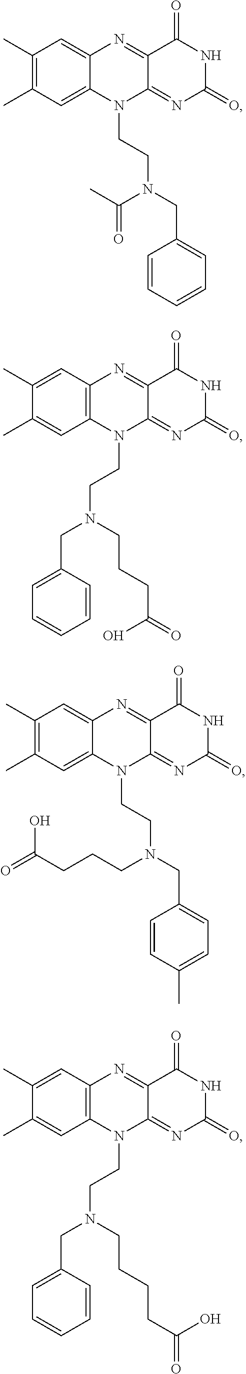

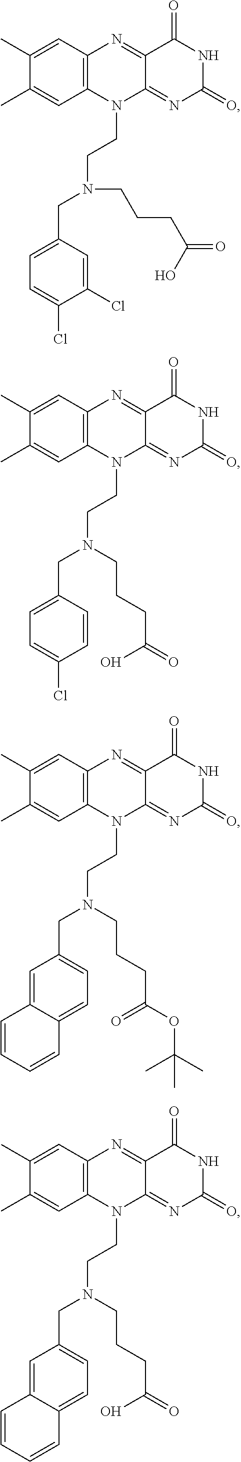

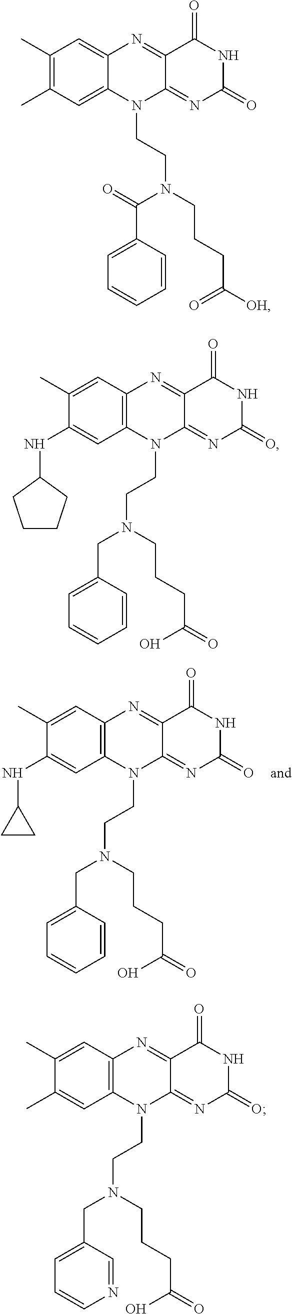

4.21 a compound of Formula IV or any of 4.1, 4.2, 4.3, 4.12-4.19, wherein said compound is selected from any one of the following:

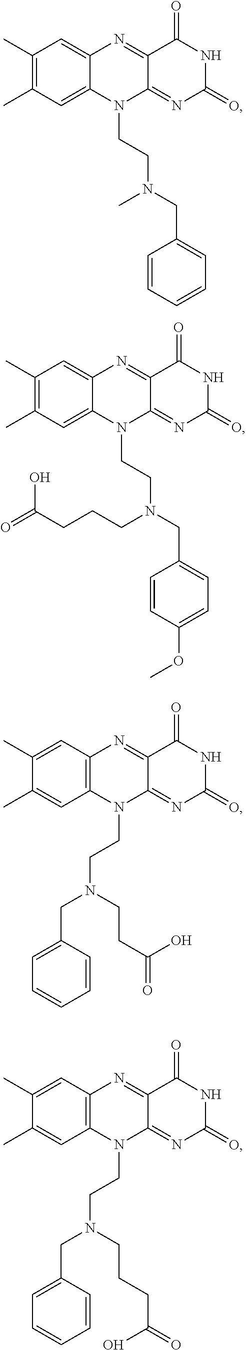

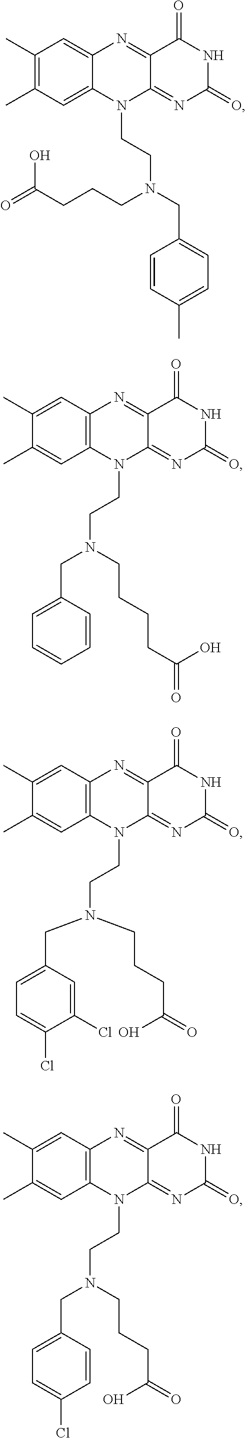

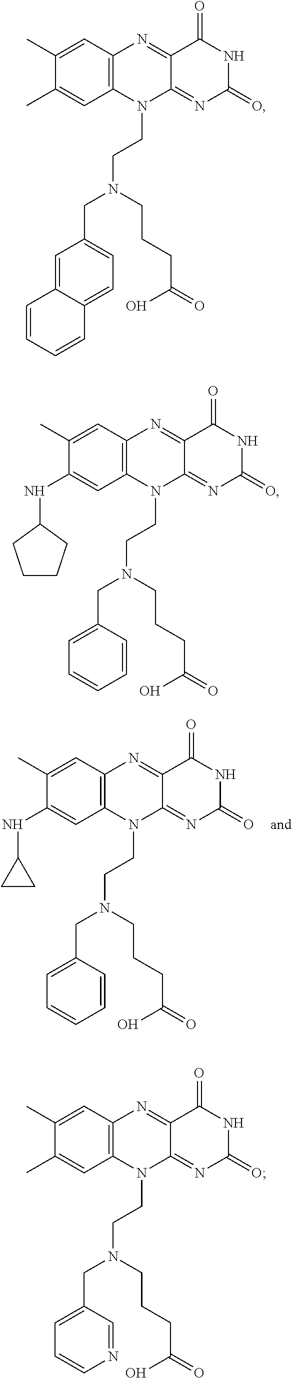

4.22 any of the preceding formulae wherein the Compound of Formula III (a) binds to FMN riboswitch, e.g., with an IC₅₀ of less than or equal to 10 μM, preferably less than 1 μM, more preferably less than 100 nM, most preferably less than 10 nM in a binding assay, for example, as described in Example 1 and/or (b) has a minimum inhibitory concentration (MIC) of less than 128 μg/mL, preferably less than 32 μg/mL, in an assay, for example, as described in Example 1A;
   in free, salt or prodrug form.

In still another embodiment, the invention relates to a compound of Formula V:

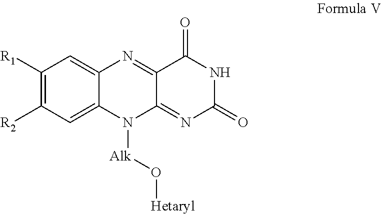

wherein Alk is C₁₋₆alkyl and hetaryl is heteroaryl (e.g., pyrimidin-2-yl) and R₁ and R₂ are independently H, C₁₋₄alkyl (e.g., methyl), in free or salt form.

In yet another embodiment, the invention relates to a compound of Formula VI:

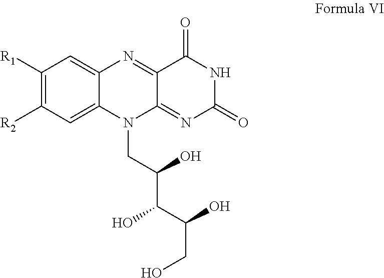

wherein R₁ is H or C₁₋₄alkyl (e.g., methyl) and R₂ is cyano, in free, salt or prodrug form.

In the first aspect, the invention provides a method for the treatment or prophylaxis of a bacterial infection (Method I) comprising administering to a subject in need thereof an effective amount of a compound of formula I, e.g., any of Methods 1.1-1.212, or a compound of any of Formulae I(i)-I(v), e.g., any of 1.213-1.265, in free, pharmaceutically acceptable salt or prodrug form, as herein before described, with the proviso that: (a) when R₁ is methyl and R₂ is chloro, then R₃ is not methyl; (b) when R₁ is H and R₂ is dimethylamine, then R₃ is not H; (c) when R₃ is (2R,3S,4S)-2,3,4,5-tetrahydroxypentyl or 5-dihydrogen phosphate (2R,3S,4S)-trihydroxypentyl, and R₁ is methyl, then R₂ is not methyl; (d) when R₃ is (2R,3S,4S)-2,3,4,5-tetrahydroxypentyl and R₁ is methyl, then R₂ is not dimethylamino; (e) when R₁ is methyl and R₂ is alkoxy, then R₃ is not 2,3,4,5-tetrahydroxypentyl; and (0 when the bacterial infection is an infection by chlamydophila psittacci, then R₃ is not —(CH₂)₂₋₆-phosphate, when R₁ and R₂ are independently selected from a group consisting of C₁₋₅ alkyl, C₁₋₅ alkoxy, amino, hydrogen and halogen group. In a further embodiment of this aspect, Method I further provides the proviso that when R₃ is 5-dihydrogen phosphate (2R,3S,4S)-trihydroxypentyl and R₁ is methyl, then R₂ is not dimethylamino.

In a preferred embodiment, Method I comprises administering to a subject in need thereof an effective amount of a compound of formula I, wherein R₃ is C₁₋₈ alkyl substituted with —COOR₉, —P(O)(OR₉)(OR₁₇), —P(O)(OR₉)(NR₁₃R₁₄), —P(O)(NR₁₃R₁₄)(NR₁₅R₁₆), —OP(O)(OR₉)(OR₁₇), —OP(O)(OR₉)(NR₁₃R₁₄), —OP(O)(NR₁₃R₁₄)(NR₁₅R₁₆), in free, pharmaceutically acceptable salt or prodrug form. In another preferred embodiment, Method I comprises administering to a subject in need thereof an effective amount of a compound of formula I, wherein R₃ is C₁₋₈ alkyl substituted with —COOR₉, —P(O)(OR₉)(OR₁₇), —P(O)(OR₉)(NR₁₃R₁₄), —P(O)(NR₁₃R₁₄)(NR₁₅R₁₆), —OP(O)(OR₉)(OR₁₇), —OP(O)(OR₉)(NR₁₃R₁₄), —OP(O)(NR₁₃R₁₄)(NR₁₅R₁₆), and at least one of R₉ and R₁₇ is C₁₋₈alkyl (e.g., methyl, ethyl or t-butyl), —C₁₋₄alkyl-OC(O)R₁₂, phenyl or Bn wherein phenyl and Bn are optionally substituted with halo or C₁₋₄alkoxy (3-chloro-phenylmethyl, 3-fluoro-phenylmethyl, 4-methoxy-3-fluorophenylmethyl), or at least one of R₁₃, R₁₄, R₁₅ and R₁₆ is —C₁₋₈alkyl-COOR₁₈ (e.g., —CH(methyl)-COOH, —CH(isopropyl)-COOH, —CH(isobutyl)-COOH, —CH(sec-butyl)-COOH), wherein the alkyl group of C₁₋₈alkyl-COOR₁₈ is optionally substituted with hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH), in free, pharmaceutically acceptable salt or prodrug form. In still another preferred embodiment, Method I comprises administering to a subject in need thereof an effective amount of a compound of formula I, wherein R₃ is C₁₋₈ alkyl substituted with —P(O)(OR₉)(NR₁₃R₁₄), —OP(O)(OR₉)(NR₁₃R₁₄), and at least one of R₉ and R₁₇ is C₁₋₈alkyl (e.g., methyl, ethyl or t-butyl), —C₁₋₄alkyl-OC(O)R₁₂, phenyl or Bn wherein phenyl and Bn are optionally substituted with halo or C₁₋₄alkoxy (3-chloro-phenylmethyl, 3-fluoro-phenylmethyl, 4-methoxy-3-fluorophenylmethyl), and at least one of R₁₃, R₁₄, R₁₅ and R₁₆ is —C₁₋₈alkyl-COOR₁₈ (e.g., —CH(methyl)-COOH, —CH(isopropyl)-COOH, —CH(isobutyl)-COOH, —CH(sec-butyl)-COOH), wherein the alkyl group of C₁₋₈alkyl-COOR₁₈ is optionally substituted with C₁₋₈allyl hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH), in free, pharmaceutically acceptable salt or prodrug form. In a particular embodiment, Method I comprises administering to a subject in need thereof an effective amount of a compound of formula 1.204, in free, pharmaceutically acceptable salt or prodrug form. In another embodiment, Method I comprises administering to a subject in need thereof an effective amount of a compound of formula 1.210, in free, pharmaceutically acceptable salt or prodrug form. In still another embodiment, Method I comprises administering to a subject in need thereof an effective amount of a compound of any of Formulae I(i)-I(v), e.g., any of 1.213-1.265, in free, pharmaceutically acceptable salt or prodrug form.

In still another embodiment of the first aspect, the invention provides a method for the treatment or prophylaxis of a bacterial infection (Method I(a)) comprising administering to a subject in need thereof an effective amount of a compound of formula III, e.g., any of formulae 3.1-3.27, in free, pharmaceutically acceptable salt or prodrug form. In a preferred embodiment, the invention provides Method I(a), wherein the compound of Formula III is a compound selected from any one of those described in formulae 3.20-3.26, in free, pharmaceutically acceptable salt or prodrug form.

In still another embodiment of the first aspect, the invention provides a method for the treatment or prophylaxis of a bacterial infection (Method I(b)) comprising administering to a subject in need thereof an effective amount of a compound of formula IV, e.g., any of formulae 4.1-4.22, in free, pharmaceutically acceptable salt or prodrug form. In a preferred embodiment, the invention provides Method I(a), wherein the compound of Formula III is a compound selected from any one of those described in formula 4.9-4.15 or 4.20-4.21, in free, pharmaceutically acceptable salt or prodrug form.

In another embodiment of the first aspect, the invention provides a method for the treatment or prophylaxis of a bacterial infection (Method I(c)) comprising administering to a subject in need thereof an effective amount of a compound of formula V, or VI, in free, pharmaceutically acceptable salt or prodrug form.

In a further embodiment, Method I, I(i) to I(v), I(a)-I(c) as hereinbefore described, are useful for the treatment or prophylaxis of a Gram-positive or Gram-negative bacterial infection (which methods shall be Method I-A, I(i)-A to I(v)-A, I(a)-A, I(b)-A and I(c)-A). In another specific embodiment, Method I, I(i)-I(v), I(a), I(b) and I(c) are useful for treating a bacterial infection including, but not limited to an infection by one or more of the following bacteria: Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis and Yersinia pestis (which methods shall be Method I-B, I(i)-B to I(v)-B, I(a)-B, I(b)-B). In addition to these bacteria, Method I, I(a), I(b) and I(c) are also useful for treating an infection by Bacillus subtilis, Streptococcus pyogenes, Borrelia burgdorferi and/or Borrelia burgdorferi bacteria. In a preferred embodiment, Method I, I(i)-I(v), I(a), I(b) and I(c) are useful for treating an infection by one or more of the following bacteria: Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii. In another preferred embodiment, Method I, I(i)-I(v), I(a), I(b) and I(c) are useful for treating an infection by the Staphylococcus aureus and/or Staphylococcus epidermidis bacteria. In a particular embodiment, Method I, I(i)-I(v), I(a), I(b) and I(c) are useful for treating a Staphylococcus aureus infection (Method I-C, I(i)-C to I(v)-C, I(a)-C, I(b)-C, I(c)-C).

In a further embodiment, Method I as hereinbefore described is useful for the treatment or prophylaxis of a disease, infection or condition selected from a group consisting of anthrax, staphylococcal scalded skin syndrome (staph infections), pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis, comprising administering to a subject in need thereof an effective amount of a Compound of Formula I, e.g., any of 1.1-1.212, or any of formulae I(i)-I(v), e.g., any of 1.213-1.265, preferably formula 1.259-1.264, in free, pharmaceutically acceptable salt or prodrug form (Method I-D).

In still another embodiment, Method I(a) as hereinbefore described is useful for the treatment or prophylaxis of a disease, infection or condition selected from a group consisting of anthrax, staphylococcal scalded skin syndrome (staph infections), pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis, comprising administering to a subject in need thereof an effective amount of a Compound of Formula III, e.g., any of 3.1-3.27, in free, pharmaceutically acceptable salt or prodrug form. (Method I(a)-D).

In yet another embodiment, Method I(b) as hereinbefore described is useful for the treatment or prophylaxis of a disease, infection or condition selected from a group consisting of anthrax, staphylococcal scalded skin syndrome (staph infections), pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis, comprising administering to a subject in need thereof an effective amount of a Compound of Formula IV, e.g., any of 4.1-4.22, preferably any of 4.9-4.15 or 4.20-4.21, in free, pharmaceutically acceptable salt or prodrug form. (Method I(b)-D).

In still another embodiment, Method I(c) as hereinbefore described is useful for the treatment or prophylaxis of a disease, infection or condition selected from a group consisting of anthrax, staphylococcal scalded skin syndrome (staph infections), pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis, comprising administering to a subject in need thereof an effective amount of a Compound of Formula V or VI, in free, pharmaceutically acceptable salt or prodrug form. (Method I(c)-D).

Without being bound to any particular theory, it is believed that various compounds of the current invention are useful in methods of treating a bacterial infection via a novel mechanism, e.g., by utilizing riboswitch-ligand binding to alter gene expression, thereby affecting downstream riboflavin biosynthesis. As such, various Compounds of the Invention, e.g., various Compounds of Formula I, e.g., various compounds of formulae 1.1-1.212, e.g., any of 1.204; various compounds of formulae I(i) —I(v), e.g., various compounds of Formulae 1.213-1.265, e.g., any of formulae 1.261 or 1.262; various compounds of formula III, e.g., various compounds of formulae 3.1-3.27, e.g., any of 3.21 or 3.22, various compounds of formula 3.23; or various compounds of formula IV, e.g., various compounds of formulae 4.1-4.22, e.g., any of formula 4.10, 4.12 or 4.21, in free, pharmaceutically acceptable salt or prodrug form, are effective in treating an infection wherein traditional antibiotics are rendered ineffective due to drug resistance. Therefore, in a particular embodiment, the invention provides Method I or any of Methods I-A to I-D as hereinbefore described wherein the infection is by an infectious agent which is resistant to a drug that is not a riboswitch ligand (Method I-E). In a further embodiment, the infection is resistant to one or more drugs selected from a group consisting of a penicillin, vancomycin, cephlorsporin and methicillin. In a particular embodiment, the infection is a methicillin-resistant Staphylococcus aureus infection. In another embodiment, the invention provides Method I(a) as hereinbefore described wherein the infection is by an infectious agent which is resistant to a drug that is not a riboswitch ligand, e.g., an infectious agent resistant to one or more drugs selected from a group consisting of a penicillin, vancomycin, cephlorsporin and methicillin, e.g., a methicillin-resistant Staphylococcus aureus infection (Method I(a)-E). In still another embodiment, the invention provides Method I(b) as hereinbefore described wherein the infection is by an infectious agent which is resistant to a drug that is not a riboswitch ligand, e.g., an infectious agent resistant to one or more drugs selected from a group consisting of a penicillin, vancomycin, cephlorsporin and methicillin, e.g., a methicillin-resistant Staphylococcus aureus infection (Method I(b)-E). In yet another embodiment, the invention provides Method I(c) as hereinbefore described wherein the infection is by an infectious agent which is resistant to a drug that is not a riboswitch ligand, e.g., an infectious agent resistant to one or more drugs selected from a group consisting of a penicillin, vancomycin, cephlorsporin and methicillin, e.g., a methicillin-resistant Staphylococcus aureus infection (Method I(c)-E).

In another embodiment, Methods I-A through I-E encompass a compound of Formula I as described in Method I, with the further proviso that when R₃ is 5-dihydrogen phosphate (2R,3S,4S)-trihydroxypentyl and R₁ is methyl, then R₂ is not dimethylamino.

In a second aspect, the invention provides a method for the treatment or prophylaxis of a fungal infection (Method II) comprising administering to a subject in need thereof an effective amount of a compound of formula I, e.g., any of formulae 1.1-1.212, or any of formulae I(i)-I(v), e.g., any of 1.213-1.265, as hereinbefore described, in free, pharmaceutically acceptable salt or prodrug form, with the proviso that: (a) when R₁ is methyl and R₂ is chloro, then R₃ is not methyl; (b) when R₁ is H and R₂ is dimethylamine, then R₃ is not H; and (c) when R₁ is H or C₁₋₆ alkyl, and R₂ is hydrogen, halo, C₁₋₆alkyl, C₁₋₆alkoxy, dialkylamino or —NHCH₂CH(OH)CH(OH)CH(OH)CH₂OH, then R₃ is not H, CH₂CH₂CH(OH)CH(OH)CH₂OH, —CH₂CH₂OH, CH₂CH(OH)CH(OH)CH(OH)—CH₂OH, CH₂CH(OH)CH(OH)CH(OH)CH(OH)CH₃ or CH₂CH(OH)CH(OH)CH(OH)—CH₂OPO₃.

In a preferred embodiment, Method II comprises administering to a subject in need thereof an effective amount of a compound of formula I, wherein R₃ is C₁₋₈ alkyl substituted with —COOR₉, —P(O)(OR₉)(OR₁₇), —P(O)(OR₉)(NR₁₃R₁₄), —P(O)(NR₁₃R₁₄)(NR₁₅R₁₆), —OP(O)(OR₉)(OR₁₇), —OP(O)(OR₉)(NR₁₃R₁₄), —OP(O)(NR₁₃R₁₄)(NR₁₅R₁₆), and at least one of R₉ and R₁₇ is C₁₋₈ alkyl (e.g., methyl, ethyl or t-butyl), —C₁₋₄alkyl-OC(O)R₁₂, phenyl or Bn wherein phenyl and Bn are optionally substituted with halo or C₁₋₄alkoxy (3-chloro-phenylmethyl, 3-fluoro-phenylmethyl, 4-methoxy-3-fluorophenylmethyl), or at least one of R₁₃, R₁₄, R₁₅ and R₁₆ is —C₁₋₈alkyl-COOR₁₈ (e.g., —CH(methyl)-COOH, —CH(isopropyl)-COOH, —CH(isobutyl)-COOH, —CH(sec-butyl)-COOH), wherein the alkyl group of C₁₋₈alkyl-COOR₁₈ is optionally substituted with hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH), in free, pharmaceutically acceptable salt or prodrug form. In still another preferred embodiment, Method II comprises administering to a subject in need thereof an effective amount of a compound of formula I, wherein R₃ is C₁₋₈alkyl substituted with —P(O)(OR₉)(NR₁₃R₁₄), —OP(O)(OR₉)(NR₁₃R₁₄), and at least one of R₉ and R₁₇ is C₁₋₈alkyl (e.g., methyl, ethyl or t-butyl), —C₁₋₄alkyl-OC(O)R₁₂, phenyl or Bn wherein phenyl and Bn are optionally substituted with halo or C₁₋₄alkoxy (3-chloro-phenylmethyl, 3-fluoro-phenylmethyl, 4-methoxy-3-fluorophenylmethyl), and at least one of R₁₃, R₁₄, R₁₅ and R₁₆ is —C₁₋₈alkyl-COOR₁₈ (e.g., —CH(methyl)-COOH, —CH(isopropyl)-COOH, —CH(isobutyl)-COOH, —CH(sec-butyl)-COOH), wherein the alkyl group of C₁₋₈alkyl-COOR₁₈ is optionally substituted with C₁₋₈alkyl hydroxyC₁₋₈alkyl (e.g., —CH(hydroxymethyl)-COOH), carboxyC₁₋₈alkyl (e.g., —CH(—CH₂COOH)—COOH or —CH(CH₂CH₂COOH)—COOH), in free, pharmaceutically acceptable salt or prodrug form. In yet another preferred embodiment, Method II comprises administering to a subject in need thereof an effective amount of a compound of formula 1.204 in free, pharmaceutically acceptable salt or prodrug form. In another preferred embodiment, Method I comprises administering to a subject in need thereof an effective amount of a compound of formula 1.210, in free, pharmaceutically acceptable salt or prodrug form. In still another embodiment, Method II comprises administering to a subject in need thereof an effective amount of a compound of formula 1.259-1.261, in free, pharmaceutically acceptable salt or prodrug form.

In another embodiment of the second aspect, the invention provides a method for the treatment or prophylaxis of a fungal infection (Method II(a)) comprising administering to a subject in need thereof an effective amount of a compound of formula III, e.g., any of 3.1-3.27, in free, pharmaceutically acceptable salt or prodrug form. In a preferred embodiment, Method II(a) comprises administering to a subject in need thereof an effective amount of a compound selected from any of those set forth in formula 3.21, in free, pharmaceutically acceptable salt or prodrug form.

In still another embodiment of the second aspect, the invention provides a method for the treatment or prophylaxis of a fungal infection (Method II(b)) comprising administering to a subject in need thereof an effective amount of a compound of formula IV, e.g., any of 4.1-4.22, in free, pharmaceutically acceptable salt or prodrug form. In a preferred embodiment, Method II(b) comprises administering to a subject in need thereof an effective amount of a compound selected from any of those set forth in any of formulae 4.9-4.15 or 4.20-4.21, in free, pharmaceutically acceptable salt or prodrug form.

In yet another embodiment of the second aspect, the invention provides a method for the treatment or prophylaxis of a fungal infection (Method II(c)) comprising administering to a subject in need thereof an effective amount of a compound of formula V or VI, in free, pharmaceutically acceptable salt or prodrug form.

In a third aspect, the invention provides use of a Compound of Formula I, e.g., any of 1.1-1.212, or any of formulae I(i)-I(v), e.g., any of 1.213-1.265, as hereinbefore described in Method I, in free, pharmaceutically acceptable salt or prodrug form, in the manufacture of a medicament for the treatment or prophylaxis of a bacterial infection. In a further aspect, the invention provides use of a Compound of Formula I, with the further proviso that when R₃ is 5-dihydrogen phosphate (2R,3S,4S)-trihydroxypentyl and R₁ is methyl, then R₂ is not dimethylamino as hereinbefore described in Method I in free, pharmaceutically acceptable salt or prodrug form, in the manufacture of a medicament for the treatment or prophylaxis of a bacterial infection. In another embodiment of the third aspect, the invention provides use of a Compound of Formula III, e.g., any of 3.1-3.27, preferably a compound selected from any of those set forth in formula 3.21, in free, pharmaceutically acceptable salt or prodrug form, in the manufacture of a medicament for the treatment or prophylaxis of a bacterial infection. In still another embodiment of the third aspect, the invention provides use of a Compound of Formula IV, e.g., any of 4.1-4.22, preferably a compound selected from any of those set forth in any of formulae 4.9-4.15 or 4.20-4.21, in free, pharmaceutically acceptable salt or prodrug form, in the manufacture of a medicament for the treatment or prophylaxis of a bacterial infection. In yet another embodiment of the third aspect, the invention provides use of a Compound of Formula V or VI, in free, pharmaceutically acceptable salt or prodrug form, in the manufacture of a medicament for the treatment or prophylaxis of a bacterial infection. In another specific embodiment, the infection is a Gram-positive or Gram-negative infection. In still another specific embodiment, the infection is an infection of one or more bacteria selected from a group consisting of Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis and Yersinia pestis. In addition to these bacteria, use of the compounds of the invention as hereinbefore described may also be for the treatment of an infection by the Bacillus subtilis, Streptococcus pyogenes and/or Borrelia burgdorferi bacteria. In a preferred embodiment, the bacteria is selected from any one of the following: Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii. In another preferred embodiment, the infection is by the Staphylococcus aureus and/or Staphylococcus epidermidis bacteria. In a further embodiment, the invention provides use as herein described in the manufacture of a medicament for the treatment or prophylaxis of a condition, disease or infection selected from anthrax, staphylococcal scalded skin syndrome (staph infections), pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis.

In yet another embodiment, the invention provides use of various Compounds of Formula I, e.g., various compounds of formulae 1.1-1.212, e.g., any of 1.204, or various compounds of formulae I(i)-I(v), e.g., various compounds of formulae 1.213-1.265, e.g., any of formula 1.261 or 1.262, as hereinbefore described in Methods I (i.e., use as hereinbefore described), wherein said infection is resistant to a drug that is not a riboswitch ligand. In a further embodiment, the invention provides use of a Compound of Formula I, with the further proviso that when R₃ is 5-dihydrogen phosphate (2R,3S,4S)-trihydroxypentyl and R₁ is methyl, then R₂ is not dimethylamino as hereinbefore described in Method I, in free, pharmaceutically acceptable salt or prodrug form, wherein said infection is resistant to a drug that is not a riboswitch ligand. In another further embodiment, the infection is resistant to one or more drugs selected from a group consisting of penicillin, vancomycin, cephlorsporin and methicillin. In a particular embodiment, the infection is a methicillin-resistant Staphylococcus aureus infection.

In still another embodiment, the invention provides use of a Compound of Formula III, e.g., various compounds of formulae 3.1-3.27, e.g., any of 3.21, in free, pharmaceutically acceptable salt or prodrug form, in the manufacture of a medicament for the treatment or prophylaxis of a bacterial infection as hereinbefore described, wherein the infection is by an infectious agent which is resistant to a drug that is not a riboswitch ligand, e.g., an infectious agent resistant to one or more drugs selected from a group consisting of a penicillin, vancomycin, cephlorsporin and methicillin, e.g., a methicillin-resistant Staphylococcus aureus infection. In yet another embodiment, the invention provides use of various Compound of Formula IV, e.g., various compounds of formulae 4.1-4.22, e.g., any of formula 4.10, 4.12 or 4.21, in free, pharmaceutically acceptable salt or prodrug form, in the manufacture of a medicament for the treatment or prophylaxis of a bacterial infection as hereinbefore described, wherein the infection is by an infectious agent which is resistant to a drug that is not a riboswitch ligand, e.g., an infectious agent resistant to one or more drugs selected from a group consisting of a penicillin, vancomycin, cephlorsporin and methicillin, e.g., a methicillin-resistant Staphylococcus aureus infection. In still another embodiment, the invention provides use of a Compound of Formula V or VI, wherein the infection is by an infectious agent which is resistant to a drug that is not a riboswitch ligand, e.g., an infectious agent resistant to one or more drugs selected from a group consisting of a penicillin, vancomycin, cephlorsporin and methicillin, e.g., a methicillin-resistant Staphylococcus aureus infection

In a fourth aspect, the invention provides use of a Compound of Formula I, e.g., any of 1.1-1.212 or any of formulae I(i)-I(v), e.g., any of 1.213-1.265, preferably formula 1.260 or 1.261, as hereinbefore described in Method II, in free, pharmaceutically acceptable salt or prodrug form, in the manufacture of a medicament for the treatment or prophylaxis of a fungal infection. In another embodiment of the fourth aspect, the invention provides use of a Compound of Formula III, e.g., any of 3.1-3.27, in free, pharmaceutically acceptable salt or prodrug form, in the manufacture of a medicament for the treatment or prophylaxis of a fungal infection. In another embodiment of the fourth aspect, the invention provides use of a Compound of Formula IV, e.g., any of 4.1-4.22, in free, pharmaceutically acceptable salt or prodrug form, in the manufacture of a medicament for the treatment or prophylaxis of a fungal infection. In still another embodiment of the fourth aspect, the invention provides use of a Compound of Formula V or VI, e.g., in free, pharmaceutically acceptable salt or prodrug form, in the manufacture of a medicament for the treatment or prophylaxis of a fungal infection

In a fifth aspect, the invention provides a pharmaceutical composition comprising a Compound of Formula I, e.g., any of 1.1-1.212, preferably 1.203-1.211, or any of formulae I(i)-I(v), e.g., any of 1.213-1.265, preferably formula 1.259-1.264 as hereinbefore described in any of Method I or II, in free, pharmaceutically acceptable salt form, in admixture with a pharmaceutically acceptable diluent or carrier.

In an sixth aspect, the invention provides a method for the treatment of an infection in a plant comprising administering to such plant an effective amount of a Compound of Formula I, e.g., any of 1.1-1.212, as hereinbefore described, preferably, 1.203-1.211, or any of formulae I(i)-I(v), e.g., any of 1.213-1.265, preferably formula 1.259-1.264, in free, salt or prodrug form. In another embodiment of the sixth aspect, the invention provides a method for the treatment of an infection in a plant comprising administering to such plant an effective amount of a Compound of Formula III, e.g., any of 3.1-3.27, as hereinbefore described, preferably 3.21-3.26, in free salt or prodrug form. In still another embodiment of the sixth aspect, the invention provides a method for the treatment of an infection in a plant comprising administering to such plant an effective amount of a Compound of Formula IV, e.g. any of 4.1-4.22, as hereinbefore described, preferably 4.10-4.15 or 4.20-4.21, in free, salt or prodrug form. In yet another embodiment of the sixth aspect, the invention provides a method for the treatment of an infection in a plant comprising administering to such plant an effective amount of a Compound of Formula V or VI in free, pharmaceutically acceptable salt form, as hereinbefore described. In another embodiment, the infection is a bacterial or fungal infection.

In a seventh aspect, the invention provides novel compound of Formula II, which comprises Compounds of Formula I, e.g., any of 1.1-1.212, as hereinbefore described in Method I or II, in free or salt form, which compound further comprises the following proviso:
(a) when R₁ is methyl and R₂ is chloro, then R₃ is not methyl;
(b) when R₁ is H and R₂ is dimethylamine, then R₃ is not H;
(c) when R₃ is (2R,3S,4S)-2,3,4,5-tetrahydroxypentyl or 5-dihydrogen phosphate (2R,3S,4S)-trihydroxypentyl, and R₁ is methyl, then R₂ is not methyl;
(d) when R₃ is (2R,3S,4S)-2,3,4,5-tetrahydroxypentyl and R₁ is methyl, then R₂ is not dimethylamino;
(e) when R₁ is methyl and R₂ is alkoxy, then R₃ is not 2,3,4,5-tetrahydroxypentyl;
(f) when R₁ and R₂ are independently selected from a group consisting of C₁₋₅ alkyl, C₁₋₅ alkoxy, amino, hydrogen and halogen group, R₃ is not —(CH₂)₂₋₆-phosphate.
(g) when R₃ is 5-dihydrogen phosphate (2R,3S,4S)-trihydroxypentyl and R₁ is methyl, then R₂ is not dimethylamino;
(h) when R₃ is —(CH₂)₂₋₆-phosphate or —(CH₂)₂₋₆-(sodium phosphate), then R₁ and R₂ are not C₁₋₅alkyl, C₁₋₅alkoxy, amino, hydrogen or halogen group;
(i) when R₁ is H or C₁₋₆alkyl, and R₂ is hydrogen, halo, C₁₋₆alkyl, C₁₋₆alkoxy, dialkylamino or —NHCH₂CH(OH)CH(OH)CH(OH)CH₂OH, then R₃ is not H, CH₂CH₂CH(OH)CH(OH)CH₂OH, —CH₂CH₂OH, CH₂CH(OH)CH(OH)CH(OH)—CH₂OH, CH₂CH(OH)CH(OH)CH(OH)CH(OH)CH₃ or CH₂CH(OH)CH(OH)—CH(OH)CH₂OPO₃;
(j) when R₁ and R₂ are both H, R₃ is not —(CH₂)₀₋₂CH₂—N(R′)₂ or —(CH₂)₀₋₂CH₂—N⁺(R′)₃—X⁻ wherein R′ is H or alkyl and X is Cl⁻, F⁻, oxalate, methosulfate, Br⁻;
(k) when R₁ is H and R₂ is chloro, then R₃ is not -alkyl-N(R^{a})(R^{b}) wherein R^{a} is alkyl and R^{b} is hydroxyalkyl;
(l) when R₁ and R₂ and are selected from H, amine, polyamine, halogen, saccharide or C₁₋₄ alkyl wherein the C atoms of the alkyl group may be replaced with N or O, wherein said alkyl group may be substituted with halogen, OH, NH₂, COOH, OR^{d}, NR^{d}R^{e}, CONR^{d}R^{e}, wherein R^{d} and R^{e} are independently alkyl, and aryl group, then R₃ is not H, amine, polyamine, halogen, saccharide or C₁₋₇ alkyl wherein the C atoms of the alkyl group may be replaced with N or O, wherein said alkyl group may be substituted with halogen, OH, NH₂, COOH, OR^{d}, NR^{d}R^{e}, CONR^{d}R^{e}, wherein R^{d} and R^{e} are independently alkyl, or aryl group;
(m) when R₁ is methyl and R₂ is —N(H)CH₃, then R₃ is not —CH₂—(CHOH)₃—CH₂OH;
(n) when R₁ and R₂ are both ethyl, then R₃ is not —CH₂—(CHOH)₃—CH₂OH;
(o) when R₁ and R₂ are methyl, then R₃ is not H, alkyl, polysaccharide or an alkyl etherified or acylated glycoside of polysaccharide;
(p) when R₃ is H, C₁₋₇alkyl, C₁₋₇haloalkyl, C₁₋₇hydroxyalkyl, C₁₋₇aminoalkyl, C₁₋₇ carboxyalkyl, or C₅₋₂₀arylC₁₋₇alkyl, then R₁ and R₂ are not H, halo, or C₁₋₇alkyl optionally substituted with —OH, halo, —COOH, —N(R_{f})(R_{g}), or —C(O)N(R_{f})(R_{g}), wherein R_{f} and R_{g} are independently H, C₁₋₇alkyl, C₃₋₂₀heterocycle, C₅₋₂₀aryl;
(q) when R₁ and R₂ are H or lower alkyl, the R₃ is not lower alkyl; and
(r) the Compound of Formula II is not riboflavin 5′-(hydrogensulfate), 7,8-dimethyl-10-(D-allityl) isoalloxazine.

In a particular embodiment, the compound of Formula II is as follows:
2.1 Formula II, wherein R₃ is C₁₋₈alkyl (e.g., n-butyl, n-pentyl, n-propyl, n-hexyl or n-heptyl), substituted with at least one —CN, —C(O)N(H)(R₈), —OR₁₀, —C₁₋₄alkyl-OC(O)R₁₂ or —OP(O)(OR₉)(OR₁₇), wherein R₉ and R₁₇ of —OP(O)(OR₉)(OR₁₇) are independently selected from C₁₋₈ alkyl(e.g., methyl, ethyl or t-butyl), phenyl or Bn optionally substituted with halo or C₁₋₄alkoxy (3-chloro-phenylmethyl, 3-fluoro-phenylmethyl, 4-methoxy-3-fluorophenylmethyl);
   R₁₀ is —C₁₋₈alkyl-OR₁₁ wherein R₁₁ is —C₁₋₄alkyl-OC(O)R₁₂, —C₁₋₈alkyl-C(O)N(H)R₈, —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl or aryl substituted with —COOR₉;
   all the other substituents are hereinbefore described in Formula I;
2.2 Formula II, wherein R₃ is C₁₋₈alkyl (e.g., n-butyl, n-pentyl, n-propyl, n-hexyl or n-heptyl), optionally substituted with at least one —C(O)OH, —OR₁₀, —C(O)N(R₆)(R₇), or —N(R₆)(R₇);
   R₂ is C₄₋₇ heterocycle (e.g., piperazinyl or pyrrolidinyl) optionally substituted or C₃₋₇cycloalkyl substituted with C₁₋₈alkyl (e.g., 4-methyl-piperazin-1-yl) or hydroxyC₁₋₈alkyl (e.g., 4-hydroxyethyl-piperazin-1-yl);
   all the other substituents are hereinbefore described in Formula I;
2.3 Formula II, wherein R₃ is C₁₋₈alkyl (e.g., n-butyl, n-pentyl, n-propyl, n-hexyl or n-heptyl), substituted with at least one —C(O)N(R₆)(R₇), or —N(R₆)(R₇);
   R₆ and R₇ are independently:
      (i) —C(O)OR₉, —C₁₋₈alkyl-C(O)OR₉, —C₁₋₈alkyl(amine)-C(O)OR₉ wherein R₉ is C₁₋₈alkyl;
      (ii) —C₁₋₈alkyl-C(O)N(H)R₈; or
      (iii) —C₁₋₈alkyl-P(O)(OR₉)₂, C₁₋₈alkyl-OP(O)(OR₉)₂, —C₁₋₈alkyl-N(H)—S(O)₂(CF₃), 7,8-dimethyl-isoalloxazin-10-yl-ethyl or aryl optionally substituted with —COOR₉;
   all the other substituents are hereinbefore described in Formula I;
2.4 Formula II, wherein R₃ is C₁₋₈alkyl (e.g., n-butyl, n-pentyl, n-propyl, n-hexyl or n-heptyl) substituted with —OP(O)(OR₉)(OR₁₇);
   R₉ and R₁₇ of —OP(O)(OR₉)(OR₁₇) are H;
   R₂ is C₆₋₈alkyl, C₆₋₈alkoxy, —N(R₄)(R₅), C₃₋₇cycloalkyl or C₄₋₇heterocycle (e.g., piperazinyl or pyrrolidinyl) optionally substituted with C₁₋₈alkyl (e.g., 4-methyl-piperazin-1-yl) or hydroxyC₁₋₈alkyl (e.g., 4-hydroxyethyl-piperazin-1-yl); or R₁ and R₂ are connected so as to form a cyclic ring structure containing —OCH₂CH₂O—;
   R₄ and R₅ are independently C₃₋₇cycloalkyl (e.g., cyclopropyl or cyclopentyl), C₄₋₇heterocycle (e.g., piperazinyl), C₃₋₈alkyl or C₁₋₈alkyl substituted with —OH, —C(O)OR₉, —N(R₆)(R₇) (e.g., amino, dimethylaminoethyl), C₁₋₈alkoxyl (e.g., methoxy), C₆₋₁₀aryl (e.g., phenyl), C₅₋₁₀heteroaryl (e.g., pyridinyl) optionally substituted with halo (e.g., 4-fluorophenyl), or C₄₋₇heterocycle optionally substituted with C₁₋₈alkyl (e.g., morpholin-4-yl or 4-methylpiperazin-1-yl); and
   all the other substituents are hereinbefore described in Formula I;
2.5 Formula 2.4, wherein R₄ and R₅ are independently C₃₋₇cycloalkyl (e.g., cyclopropyl or cyclopentyl), C₄₋₇heterocycle (e.g., piperazinyl), C₆₋₈alkyl or C₁₋₈alkyl substituted with —OH, —C(O)OR₉, —N(R₆)(R₇) (e.g., amino, dimethylamino), C₁₋₈alkoxyl (e.g., methoxy), C₆₋₁₀aryl (e.g., phenyl), C₅₋₁₀heteroaryl (e.g., pyridinyl) optionally substituted with halo (e.g., 4-fluorophenyl), or C₄₋₇heterocycle optionally substituted with C₁₋₈alkyl (e.g., morpholin-4-yl or 4-methylpiperazin-1-yl);
2.6 Formula 2.4, wherein R₄ and R₅ are independently selected from C₃₋₇cycloalkyl (e.g., cyclopropyl or cyclopentyl), C₄₋₇heterocycle (e.g., piperazinyl), and C₁₋₈alkyl substituted with one or more groups selected from —OR₁₁, —C(O)OR₉, —N(R₆)(R₇) (e.g., amino, dimethylamino), C₁₋₈alkoxyl (e.g., methoxy), C₆₋₁₀aryl (e.g., phenyl), C₅₋₁₀heteroaryl (e.g., pyridinyl) optionally substituted with halo (e.g., 4-fluorophenyl), and C₄₋₇heterocycle optionally substituted with C₁₋₈alkyl (e.g., morpholin-4-yl or 4-methylpiperazin-1-yl);
2.7 Formula II, wherein R₃ is C₁₋₈alkyl (e.g., n-butyl, n-pentyl, n-propyl, n-hexyl or n-heptyl), substituted with one or more groups selected from —OP(O)(OR₉)(OR₁₇), —CN, —C(O)OR₉, —C(O)N(H)(R₈), —OR₁₀, —C(O)N(R₆)(R₇), or —N(R₆)(R₇);
   R₉ and R₁₇ of OP(O)(OR₉)(OR₁₇) and —C(O)OR₉ are independently C₁₋₈ alkyl (e.g., methyl, ethyl or t-butyl), phenyl or Bn optionally substituted with halo or C₁₋₄alkoxy (3-chloro-phenylmethyl, 3-fluoro-phenylmethyl, 4-methoxy-3-fluorophenylmethyl), or —C₁₋₄alkyl-OC(O)R₁₂;
   all the other substituents are hereinbefore described in Formula I;
2.8 Formula II, wherein R₃ is C₁₋₈alkyl (e.g., n-butyl, n-pentyl, n-propyl, n-hexyl or n-heptyl), substituted with at least one —OR₁₀, group wherein R₁₀ is H;
   R₂ is —N(R₄)(R₅), C₃₋₇cycloalkyl substituted with or C₄₋₇heterocycle (e.g., piperazinyl or pyrrolidinyl) optionally substituted with C₁₋₈alkyl (e.g., 4-methyl-piperazin-1-yl) or hydroxyC₁₋₈alkyl (e.g., 4-hydroxyethyl-piperazin-1-yl);
   R₄ and R₅ are independently H, C₄₋₇heterocycle (e.g., piperazinyl) substituted with C₁₋₈alkyl (e.g., morpholin-4-yl or 4-methylpiperazin-1-yl), or C₁₋₈alkyl (e.g., methyl or ethyl) substituted with —C(O)OR₉ wherein R₉ of —C(O)OR₉ is C₁₋₈alkyl (e.g., methyl, ethyl or t-butyl), —C₁₋₄alkyl-OC(O)R₁₂, phenyl or Bn wherein said phenyl or Bn are optionally substituted with halo or C₁₋₄alkoxy (3-chloro-phenylmethyl, 3-fluoro-phenylmethyl, 4-methoxy-3-fluorophenylmethyl), —N(R₆)(R₇), C₆₋₁₀aryl (e.g., phenyl) or C₅₋₁₀heteroaryl (e.g., pyridinyl) wherein said aryl or heteroaryl is substituted with halo (e.g., 4-fluorophenyl), provided that R₄ and R₅ are not both H;
   R₆ and R₇ are independently selected from:
      (i) —C(O)OR₉, —C₁₋₈alkyl-C(O)OR₉, —C₁₋₄alkyl-OC(O)R₁₂ or —C₁₋₈alkyl(amine)-C(O)OR₉ wherein R₉ of —C(O)OR₉, —C₁₋₈alkyl-C(O)OR₉ or —C₁₋₈alkyl(amine)-C(O)OR₉ is C₁₋₈alkyl (e.g., methyl, ethyl or t-butyl), phenyl or Bn optionally substituted with halo or C₁₋₄alkoxy (3-chloro-phenylmethyl, 3-fluoro-phenylmethyl, 4-methoxy-3-fluorophenylmethyl);
      (ii) —C₁₋₈alkyl-C(O)N(H)R₈; or
      (iii)-C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl or aryl optionally substituted with —COOR₉;
   all the other substituents are hereinbefore described in Formula I;
2.9 Formula 2.8, wherein R₃ is further substituted with —OP(O)(OR₉)(OR₁₇);
2.10 Formula 2.8 or 2.9, wherein R₃ is 5-phosphate-(2S,3S,4R)-2,3,4-trihydroxypentyl;
2.11 Formula II, wherein R₃ is C₁₋₈alkyl (e.g., n-butyl, n-pentyl, n-propyl, n-hexyl or n-heptyl), substituted with a —N(R₆)(R₇);
   R₂ is C₁₋₈ alkyl (e.g., methyl or ethyl), C₁₋₈alkoxy (e.g., methoxy or ethoxy), —N(R₄)(R₅), C₃₋₇ cycloalkyl or C₄₋₇heterocycle (e.g., piperazinyl or pyrrolidinyl) optionally substituted with C₁₋₈alkyl (e.g., 4-methyl-piperazin-1-yl) or hydroxyC₁₋₈alkyl (e.g., 4-hydroxyethyl-piperazin-1-yl);
   all the other substituents are hereinbefore described in Formula I;
2.12 Formula II, wherein R₃ is C₁₋₈alkyl (e.g., n-butyl, n-pentyl, n-propyl, n-hexyl or n-heptyl), optionally substituted with —C(O)OR₉ wherein R₉ of —C(O)OR₉ is C₁₋₈alkyl (e.g., methyl, ethyl or t-butyl), —C₁₋₄alkyl-OC(O)R₁₂, phenyl or Bn wherein said phenyl and Bn are optionally substituted with halo or C₁₋₄alkoxy (3-chloro-phenylmethyl, 3-fluoro-phenylmethyl, 4-methoxy-3-fluorophenylmethyl);
   R₂ is H, halo (e.g., chloro), C₁₋₈alkyl (e.g., methyl or ethyl), C₁₋₈alkoxy (e.g., methoxy or ethoxy), —N(R₄)(R₅), C₃₋₇cycloalkyl or C₄₋₇heterocycle (e.g., piperazinyl or pyrrolidinyl) optionally substituted with C₁₋₈alkyl (e.g., 4-methyl-piperazin-1-yl) or hydroxyC₁₋₈alkyl (e.g., 4-hydroxyethyl-piperazin-1-yl);
   all the other substituents are hereinbefore described in Formula I;
2.13 Formula II, wherein R₃ is C₁₋₈alkyl substituted with at least one —COOR₉, or —OP(O)(OR₉)(OR₁₇)
   R₂ is —N(R₄)(R₅), C₃₋₇cycloalkyl or C₄₋₇heterocycle (e.g., piperazinyl or pyrrolidinyl) optionally substituted with C₁₋₈alkyl (e.g., 4-methyl-piperazin-1-yl) or hydroxyC₁₋₈alkyl (e.g., 4-hydroxyethyl-piperazin-1-yl)
   R₄ and R₅ are independently selected from H, C₃₋₇cycloalkyl (e.g., cyclopropyl or cyclopentyl), C₄₋₇heterocycle (e.g., piperazinyl), and C₃₋₈alkyl wherein said alkyl is optionally substituted with one or more —OR₁₁, —C(O)OR₉, —N(R₆)(R₇) (e.g., amino, dimethylamino), C₁₋₈alkoxyl (e.g., methoxy), C₆₋₁₀aryl (e.g., phenyl) or C₅₋₁₀heteroaryl (e.g., pyridinyl) optionally substituted with halo (e.g., 4-fluorophenyl), or C₄₋₇heterocycle optionally substituted with C₁₋₈allyl (e.g., morpholin-4-yl or 4-methylpiperazin-1-yl);
   all the other substituents are hereinbefore described in Formula I;
2.14 Formula II, wherein R₃ is —C₁₋₈alkyl-C(O)OR₉ or —C₁₋₈alkyl-OP(O)(OR₉)(OR₁₇) group wherein the alkyl group is optionally substituted with —OH;
   R₉ and R₁₇ of —C₁₋₈alkyl-C(O)OR₉ and —C₁₋₈alkyl-OP(O)(OR₉)(OR₁₇) are independently C₁₋₈alkyl (e.g., methyl, ethyl or t-butyl), —C₁₋₄allyl-OC(O)R₁₂, phenyl or Bn where in said phenyl and Bn are optionally substituted with halo or C₁₋₄alkoxy (3-chloro-phenylmethyl, 3-fluoro-phenylmethyl, 4-methoxy-3-fluorophenylmethyl);
   all the other substituents are hereinbefore described in Formula I;
2.15 Formula II, wherein R₃ is —C₁₋₈alkyl substituted with one or more groups selected from OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), or —P(O)(NR₁₃R₁₄)(NR₁₅R₁₆),
   all the other substituents are hereinbefore described in Formula I;
2.16 Formula 2.15, wherein R₃ is further substituted with one or more —OR₁₀ wherein R₁₀ is H;
   all the other substituents are hereinbefore described in Formula I;
2.17 A compound of Formula II, selected from:

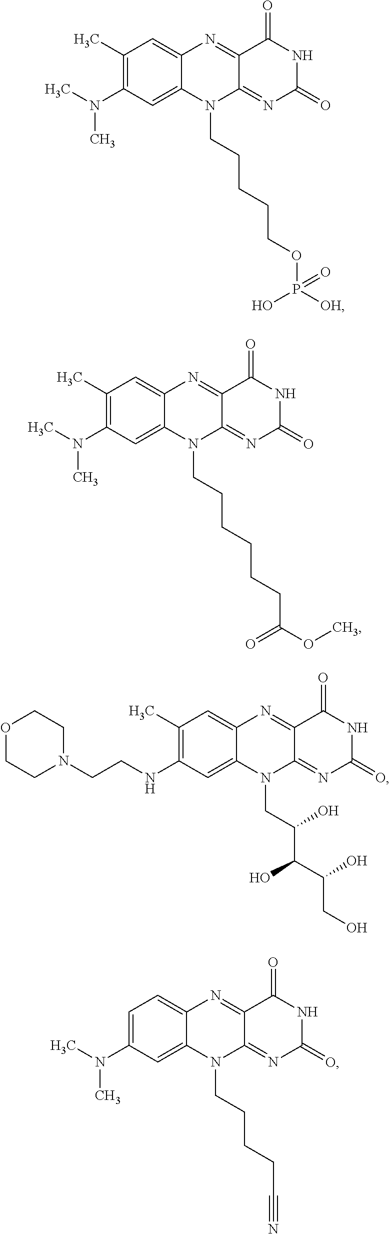

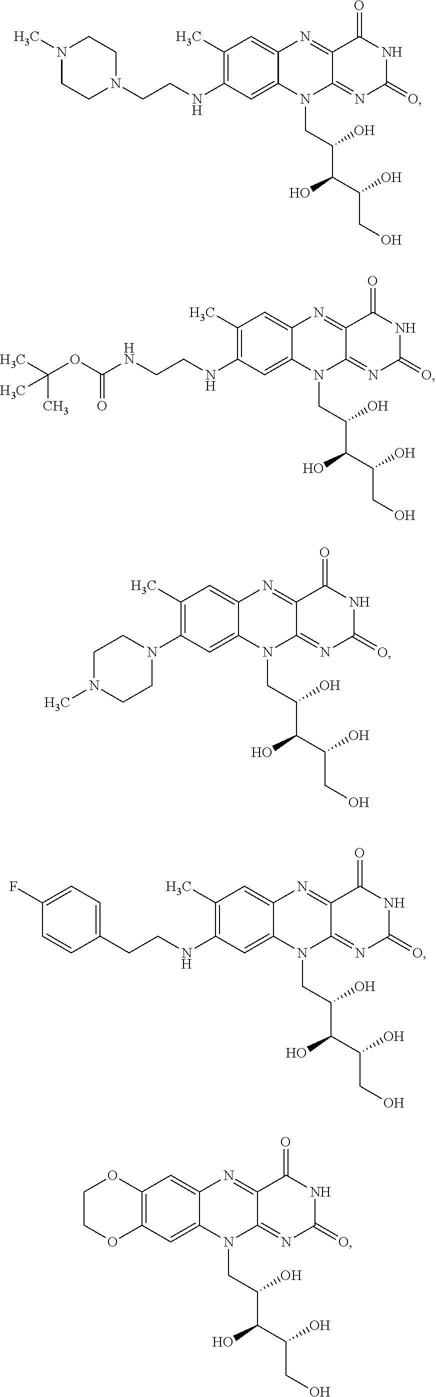

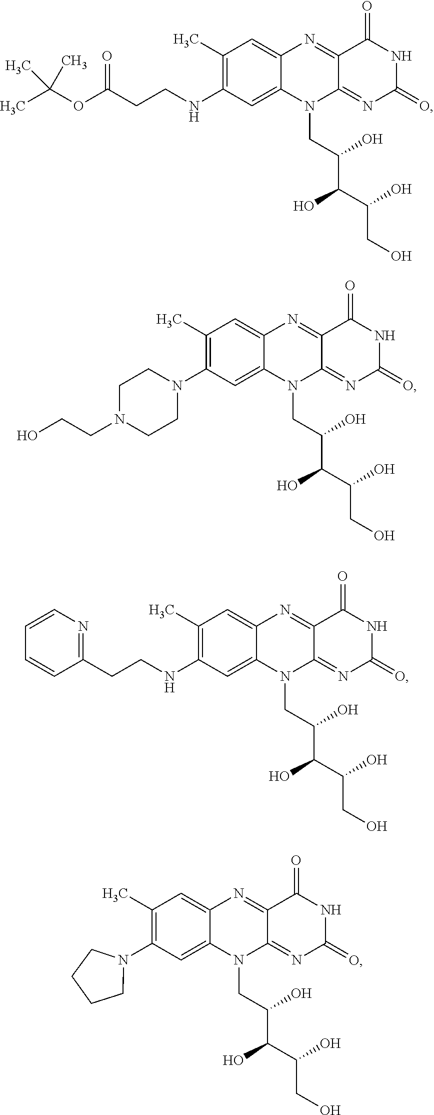

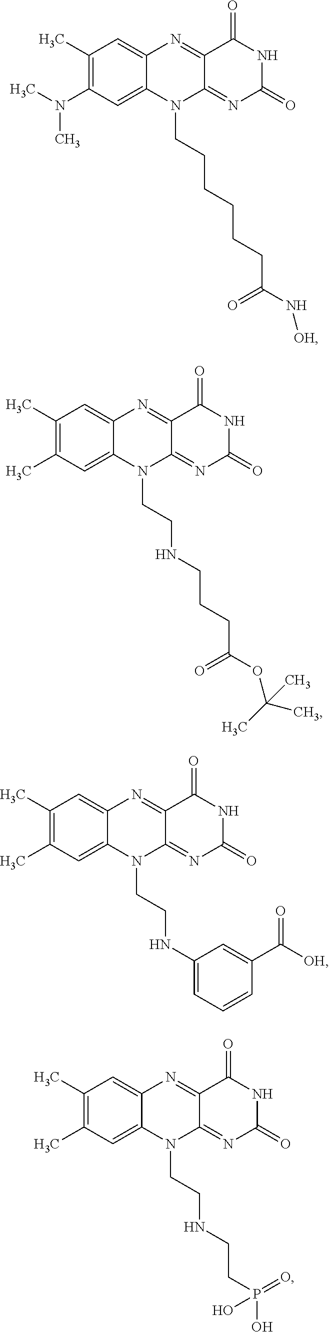

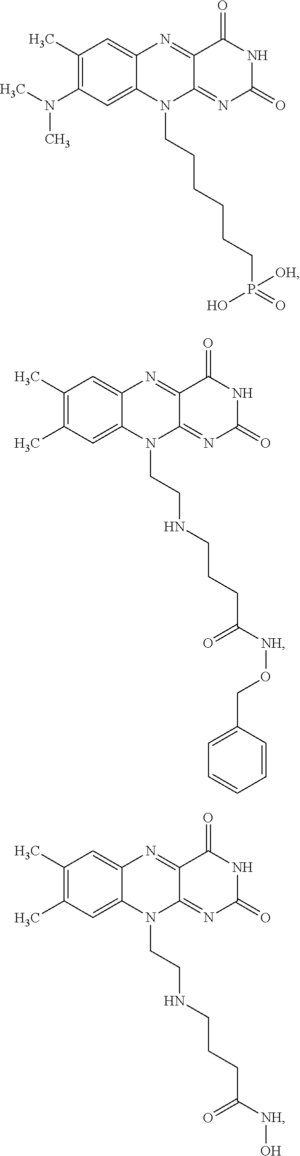

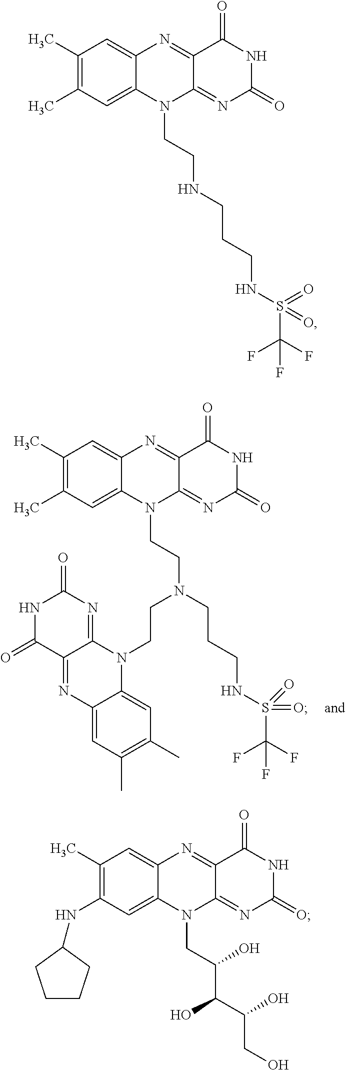

in free, salt or prodrug form.

In another embodiment, novel compound of Formula II or any of 1.1-1.202, 1.209 or any of 2.1-2.17 bind to FMN riboswitch, e.g., with an IC₅₀ of less than or equal to 10 μM, preferably less than 1 μM, more preferably less than 100 nM, most preferably less than 10 nM in a binding assay, for example, as described in Example 1. In an eighth aspect, the invention provides a novel compound wherein said compound is or any of formulae I(i)-I(v), e.g., any of 1.213-1.265, preferably formula 1.259, 1.260 or 1.261, in free, salt or prodrug form. In a further embodiment, the invention provides novel compound selected from any compounds disclosed in Table 1, in free, salt or prodrug form.

In still another embodiment of the eighth aspect, the invention provides novel compound of Formula III, e.g., any of 3.1-3.27, preferably a compound selected from any of those set forth in formula 3.20 or 3.21, in free, salt or prodrug form. In yet another embodiment of the eighth aspect, the invention provides novel compound of Formula IV, e.g., any of 4.1-4.22, preferably a compound selected from any of those set forth in formula 4.9 or 4.20, in free, salt or prodrug form. In another embodiment of the eighth aspect, the invention provides novel compound of Formula V or VI, free, salt or prodrug form.

In a ninth aspect, the invention provides a Compound of Formula II, e.g., any of 2.1-2.17 as novel FMN riboswitch ligand. The invention also provides a compound of or any of formulae I(i)-I(v), e.g., any of 1.213-1.265, preferably formula 1.261 or 1.262, in free, salt or prodrug form as novel FMN riboswitch ligand.

In another aspect, the invention provides a riboswitch ligand which comprises a compound of Formula I, e.g., any of 1.1-1.212 or any of formulae I(i)-I(v), e.g., any of 1.213-1.265, or Formula II, or any of 2.1-2.17, in free or salt form. In another embodiment, the riboswitch ligand of the invention bind to FMN riboswitch, e.g., with an IC₅₀ of less than or equal to 10 μM, preferably less than 1 μM, more preferably less than 100 nM, most preferably less than 10 nM in a binding assay, for example, as described in Example 1. In a further embodiment, the invention provides a riboswitch ligand selected from:

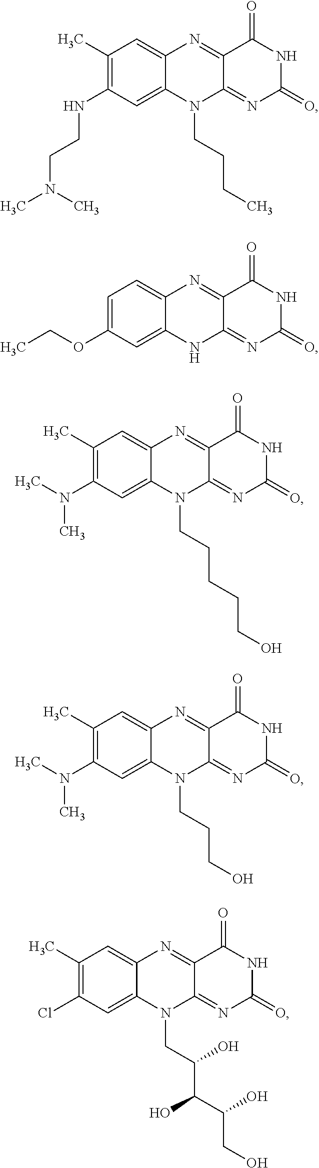

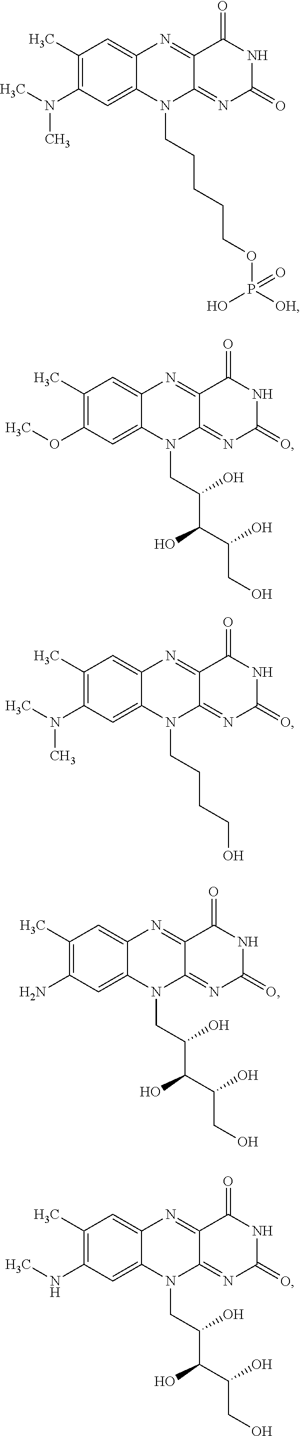

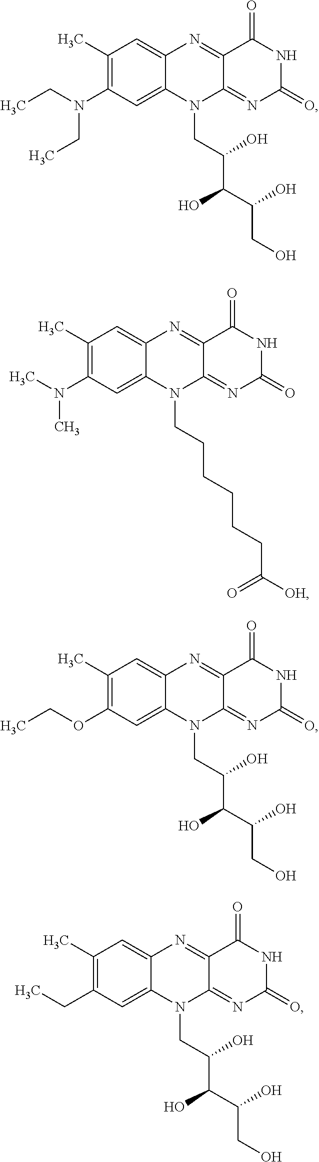

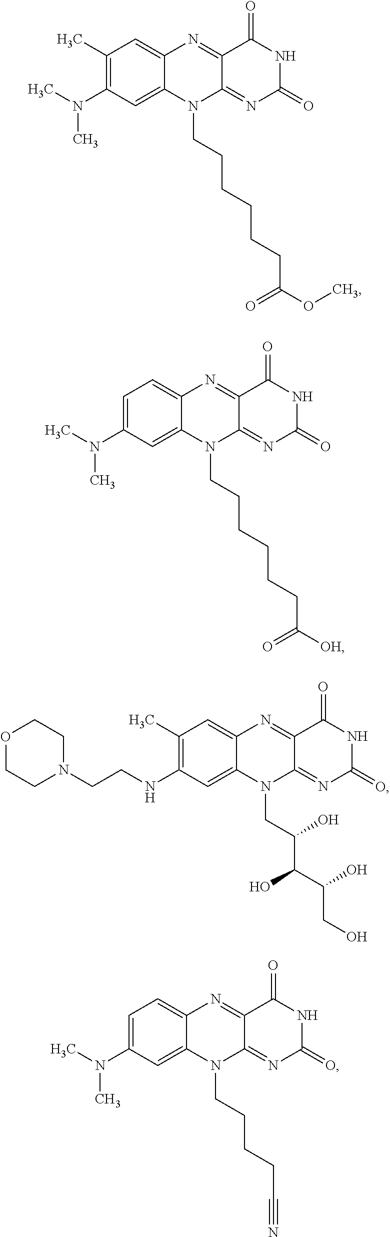

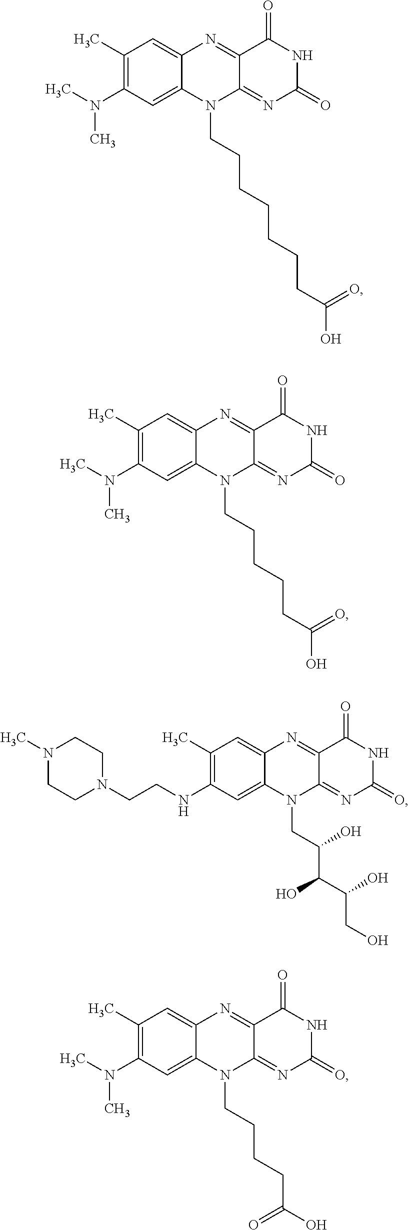

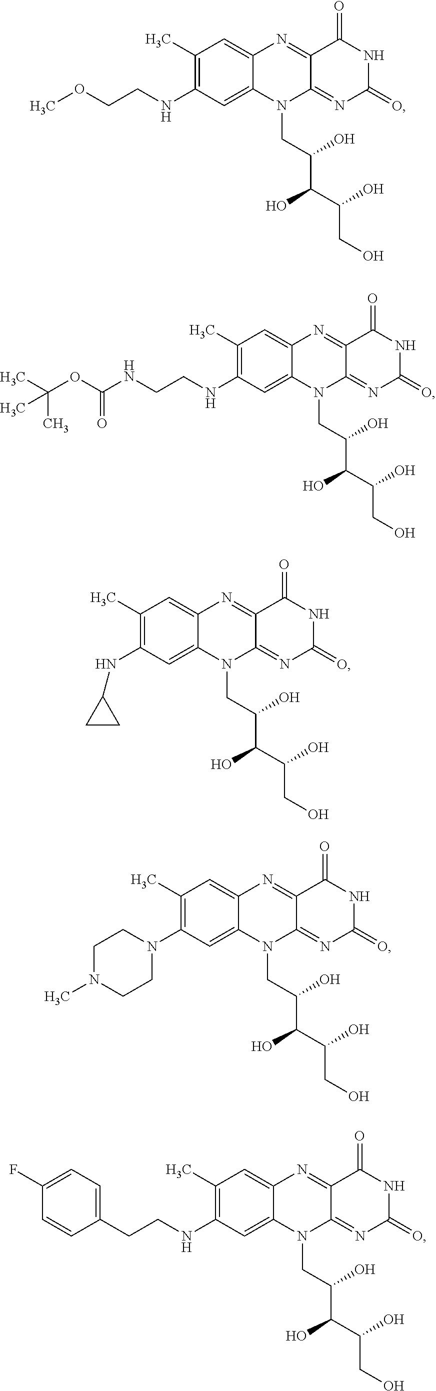

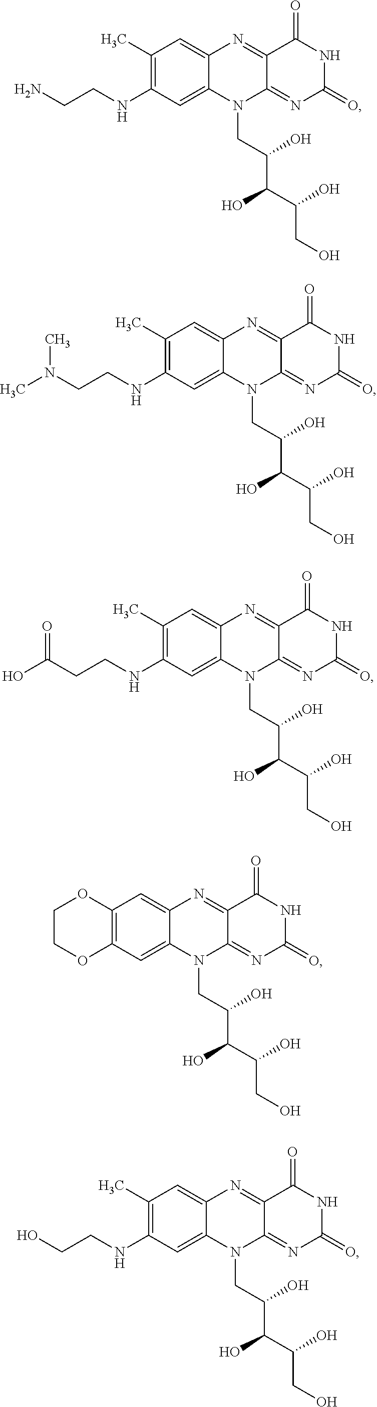

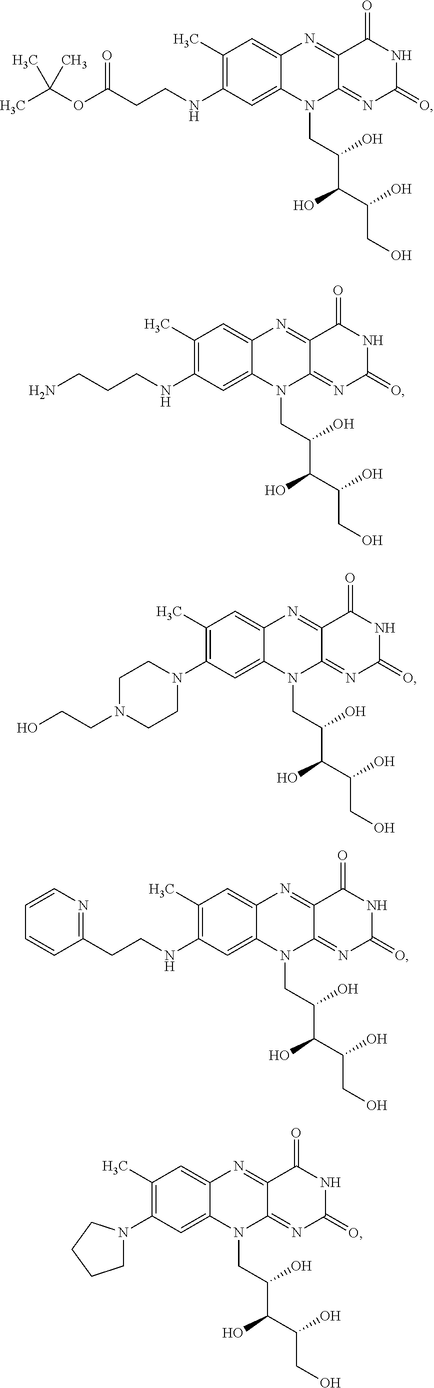

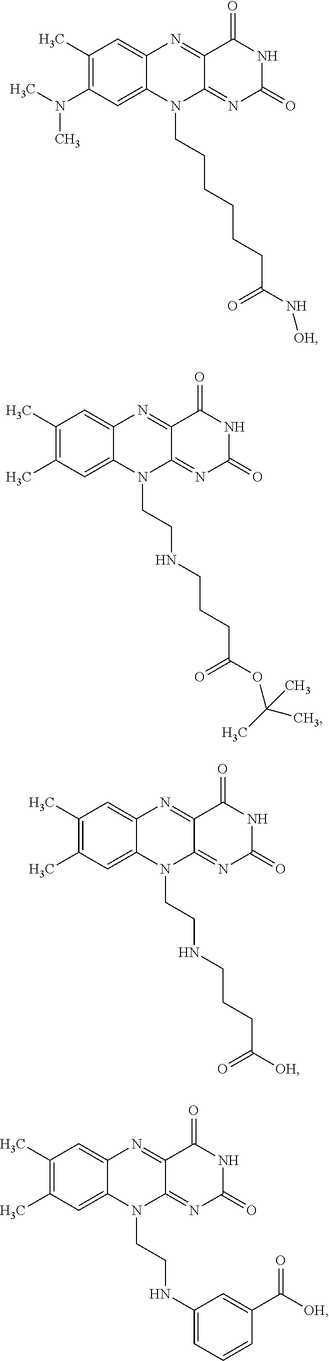

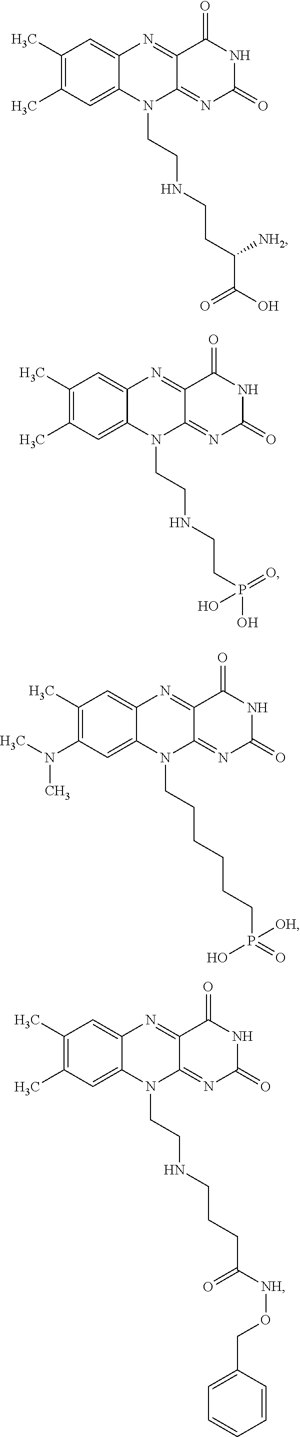

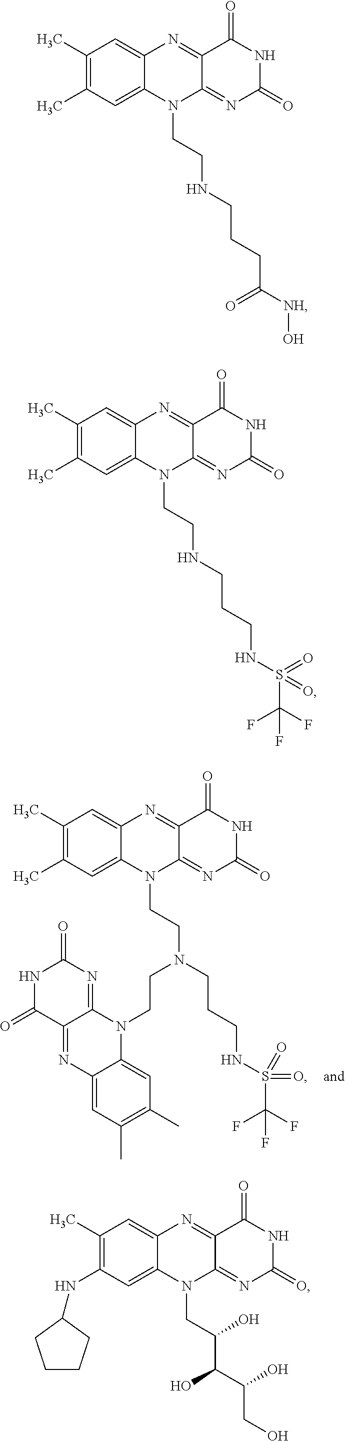

in free or salt form.

In another embodiment, the riboswich ligand is preferably a compound selected from those set forth in formula 1.261 or 1.262, in free or salt form.

In another embodiment of the ninth aspect, the invention provides a riboswitch ligand which comprises a compound of Formula III, e.g., any of 3.1-3.27, preferably a compound selected from any of those set forth in formula 3.21, in free, salt or prodrug form. In another embodiment, the riboswitch ligand of the invention binds to FMN riboswitch, e.g., with an IC₅₀ of less than or equal to 10 μM, preferably less than 1 μM, more preferably less than 100 nM, most preferably less than 10 nM in a binding assay, for example, as described in Example 1.

In yet another embodiment of the ninth aspect, the invention provides a riboswitch ligand which comprises a compound of Formula IV, e.g., any of 4.1-4.22, preferably a compound selected from any of those set forth in formula 4.10, 4.12 or 4.21, in free, salt or prodrug form.

In still another embodiment of the nineth aspect, the invention provides a riboswitch ligand which comprises a compound of Formula V or VI, in free, salt or prodrug form.

In the tenth aspect, the invention provides a pharmaceutical composition comprising a compound of the invention, e.g., any of formula I, e.g., any of formulae 1.1-1.212, or any of formulae I(i)-I(v), e.g., any of 1.213-1.265, preferably formula 1.259-1.264, or formula II, e.g., any of 2.1-2.17, in free, pharmaceutically acceptable salt form, in admixture with a pharmaceutically acceptable diluent or carrier.

In another embodiment of the tenth aspect, the invention provides a pharmaceutical composition comprising a Compound of Formula III, e.g., any of 3.1-3.27, in free, pharmaceutically acceptable salt or prodrug form, in admixture with a pharmaceutically acceptable diluents or carrier. In still another embodiment of the tenth aspect, the invention provides a pharmaceutical composition comprising a Compound of Formula IV, e.g., any of 4.1-4.22, in free, pharmaceutically acceptable salt or prodrug form, in admixture with a pharmaceutically acceptable diluents or carrier. In yet another embodiment of the tenth aspect, the invention provides a pharmaceutical composition comprising a Compound of Formula V or VI, in free, pharmaceutically acceptable salt or prodrug form, in admixture with a pharmaceutically acceptable diluents or carrier.

In still another aspect, the invention provides a method of preparing a Compound of formula I, e.g., any of formulae 1.1-1.212, or any of formulae I(i)-I(v), e.g., any of 1.213-1.265, or formula II comprising the step of reacting Int-4 as described below with a pyrimidine-2,4,5,6(1H,3H)-tetrone in the presence of boron oxide and an acid, e.g., mineral acid, e.g., acetic acid.

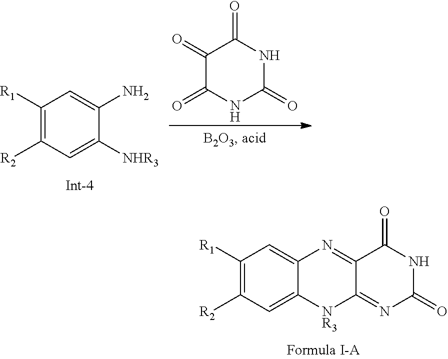

In still another embodiment, the invention provides a method of preparing a compound of formula I or II comprising the step of reacting Int-4A as described below with a violuric acid at elevated temperature (e.g., greater than 25° C., e.g., about 95° C.).

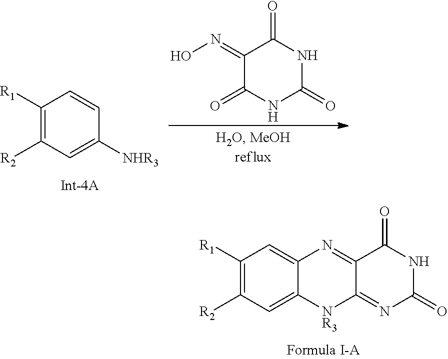

The invention further provides a method of preparing a compound of formula I or II as described below in Methods of Making Compounds of the Invention. In a further aspect, the invention provides a method of preparing a compound of formula I or II selected from any of the methods as described in any of Examples 2-92.

### DETAILED DESCRIPTION OF THE INVENTION

The term “riboswitch” or “riboswitches” is an art recognized term and refers to an mRNA which comprises a natural aptamer that binds target metabolite and an expression platform which changes in the RNA structure to regulate genes. The term “FMN riboswitch” refers to a riboswitch that binds a metabolite such as flavin mono-nucleotide (FMN) or ligands such as various Compounds of Formula I or II, e.g., various compounds of formulae 1.1-1.212, 2.1-2.17, various compounds of formulae I(i)-I(v), e.g., various compounds of formulae 1.213-1.265, various Compounds of Formula III, e.g., various compounds of formulae 3.1-3.27, various Compounds of Formula IV, e.g., various compounds of formulae 4.1-4.22, or various Compounds of Formula V or VI, e.g., any of the compounds in formulae 1.204, 1.210, 1.261, 1.262, 3.21, 4.10, 4.12 or 4.21, in free, salt or prodrug form and which affects downstream FMN biosynthesis and transport proteins.

“FMN riboswitch ligand” refers to any compound such as compounds of Formula I or II, e.g., various compounds of formulae 1.1-1.212, 2.1-2.17, formulae I(i) —I(v), e.g., various compounds of formulae 1.213-1.265, preferably formula 1.261 or 1.262, FMN or roseoflavin, or various compounds of Formula III, e.g., of formulae 3.1-3.27, various Compounds of Formula IV, e.g., of formulae of 4.1-4.22 or various Compounds of Formula V or VI, in free, salt or prodrug form which binds to the FMN riboswitch, e.g., via the FMN-binding aptamer called the RFN element, which is a highly conserved domain in the 5′-untranslated regions of prokaryotic mRNA. Without intended to be bound by any particular theory, it is believed the binding of the ligand to its riboswitch induces a conformational change in the bacterial mRNA such that the expression of the ORF is repressed, for example, such that the expression of enzymes responsible for riboflavin and FMN biosynthesis is repressed. This is achieved by inducing the mRNA to form (1) a terminator hairpin that halts RNA synthesis before the ORF can be synthesized or (2) a hairpin that sequesters the Shine-Dalgarno sequence and prevents the ribosome from binding to the mRNA so as to translate the ORF. Examples of FMN riboswitch ligands include, but are not limited to compounds of formulae 1.204, 1.210, 1.261, 1.262, 3.21, 4.10, 4.12 or 4.21, in free, salt or prodrug form.

The term “infection” encompasses any infection by bacteria and/or fungi In a particular embodiment, the term “infection” refers to a bacterial infection. In another embodiment, the infection is a Gram-positive or Gram-negative infection. In still another embodiment, the infection is an infection by one or more bacteria selected from a group consisting of Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis and Yersinia pestis. In addition, the infection is an infection by one or more bacteria selected from Bacillus subtilis, Streptococcus pyogenes and/or Borrelia burgdorferi. In a preferred embodiment, the infection is a Staphylococcus aureus and/or Staphylococcus epidermidis infection. In a further embodiment, the infection is a Staphylococcus aureus infection. In a particular embodiment, the infection is an infection which is resistant to a drug which is not a riboswitch ligand. In a further aspect of this particular embodiment, the infection is an infection which is resistant to one or more drugs selected from a group consisting of penicillin, vancomycin, cephlorsporin and methicillin. In a particular embodiment, the infection is a methicillin-resistant Staphylococcus aureus (MRSA) infection.

In other aspect, the term “infection” refers to a fungal infection. Examples of a fungal infection include but are not limited to infection by Microsporum, Trichophyton, Epidermophyton, Tinea (e.g., tinea versicolor, tinea pedis, tinea corporis), Histoplasma capsulatum, Coccidioides immitis, Blastomyces dermatidis, Candida (e.g., Candida albicans), Aspergillus, Fumigatus and Sporothrix xchenckii fungi. Examples of conditions caused by a fungal infection include, but are not limited to mycoses such as superficial, cutaneous, subcutaneous or systemic mycosis, e.g., coccidioidomycosis, histoplasmosis, blastomycosis, candidiasis (e.g., yeast infection or moniliasis), sporotrichosis and ringworm (e.g., athlete's foot, jock itch, scalp ringworm, nail ringworm, body ringworm, beard ringworm).

The term “bacteria” or “bacterial” include, but are not limited to Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis and Yersinia pestis. The term “bacteria” referred to in the current invention also includes Bacillus subtilis, Streptococcus pyogenes and/or Borrelia burgdorferi. Preferably, the bacteria referred to in the current the invention include but not limited to Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis and Streptococcus pyogenes. More preferably, the bacteria referred to in the current the invention include but not limited to Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii, Most preferably, the bacteria referred to in the current the invention include Staphylococcus aureus and/or Staphylococcus epidermidis.

If not otherwise specified or clear from context, the following terms as used herein have the following meetings:
a. “Alkyl” as used herein is a saturated or unsaturated hydrocarbon moiety, preferably saturated, e.g., one to eight or one to four carbon atoms in length, which may be linear or branched (e.g., n-butyl or tert-butyl), and may be optionally substituted, e.g., mono-, di-, or tri-substituted on any one of the carbon atom, e.g., with alkyl (e.g., methyl), alkoxy, halogen (e.g., chloro or fluoro), haloalkyl (e.g., trifluoromethyl), hydroxy, and carboxy. For example, “C₁-C₈ alkyl” denotes alkyl having 1 to 8 carbon atoms. Examples of alkyl include, but are not limited to, methyl, ethyl, n-propyl, propyl, n-butyl, i-butyl, sec-butyl, t-butyl, 3-methylpentyl, 4-methylpentyl, n-pentyl, n-hexyl and n-heptyl.
b. “Aryl” as used herein is a mono or bicyclic aromatic hydrocarbon, preferably phenyl or naphthyl, optionally substituted, e.g., with C₁-C₈alkyl (e.g., methyl), C₁-C₈alkoxy, halogen (e.g., chloro or fluoro), haloC₁-C₈alkyl (e.g., trifluoromethyl), hydroxy, carboxy, or an additional aryl or heteroaryl.
c. “Cycloalkyl” is intended to include monocyclic or polycyclic ring system comprising at least one aliphatic (non-aromatic) ring. Therefore, “cycloalkyl” may denote simply a cyclopropyl, cyclobutyl, cyclopentyl, cyclopentenyl, cyclohexyl, cyclohexenyl and the like.
d. Heterocycle as used herein refers to a monocyclic or polycyclic non-aromatic ring system wherein at least one carbon atom is replaced with a heteroatom selected from a group consisting of N, O, and S. Examples of heteroatom include morpholinyl (e.g., morpholin-4-yl), piperazinyl, piperidinyl, pyrrolidinyl and the like. Heterocycle of the invention may optionally be substituted with C₁₋₈alkyl (e.g., methyl).
e. Heteroaryl as used herein refers to a mono or bicyclic aromatic ring system comprises at least one aromatic ring containing at least one heteroatom independently selected from the group consisting of N, O and S. The heteroaryl ring may be attached to its pendant group at any heteroatom or carbon atom which results in a stable structure. The heteoraryl rings described herein may be substituted on the carbon or on the nitrogen atom if the resulting compound is stable. Examples of heteroaryl group include, but are not limited to pyridinyl (e.g., pyridine-2-yl), imidazolyl, thiazolyl, pyrazinyl, pyrimidinyl, quinoxalinyl, and the like. The heteroaryl group may also be optionally substituted with C₁₋₈alkyl (e.g., methyl), C₁₋₈alkoxy, halogen, hydroxy, haloalkyl or carboxy.

The substituents on the Compounds of the Invention, e.g., R₁-R₁₈ may be specifically or generally defined. Unless specified otherwise, R₁-R₁₈ are defined as in Formula I, II, III or IV. In other instances, R₁-R₁₈ are defined by the embodiment or claims to which it depends.

The Compounds of the Invention (e.g., Compounds of Formula I, e.g., any of 1.1-1.212 or any of formulae I(i)-I(v), e.g., any of 1.213-1.265, as described in any of Methods I or II; Compounds of Formula II, or any of 2.1-2.17; a Compound of Formula III, e.g., any of 3.1-3.27; a Compound of Formula IV, e.g., any of 4.1-4.22, or a Compound of Formula V or VI, as hereinbefore described, or a compound in Examples 2-92, as hereinafter described) may exist in free or salt form, e.g., as acid addition salts. An acid-addition salt of a compound of the invention which is sufficiently basic, for example, an acid-addition salt with, for example, an inorganic or organic acid, for example hydrochloric, hydrobromic, sulphuric, phosphoric, acid acetic, trifluoroacetic, citric, maleic acid, toluene sulfonic, propionic, succinic, glycolic, stearic, lactic, malic, tartaric, citric, ascorbic, pamoic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid, and the like. In addition a salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In this specification, unless otherwise indicated, language such as Compounds of the Invention is to be understood as embracing such Compounds of Formula I (e.g., any of 1.1-1.212 or any of formulae I(i)-I(v), e.g., any of 1.213-1.265, preferably formula 1.259, 1.260 or 1.261 as described in any of Methods I or II), Compounds of formula II (e.g., any of 2.1-2.17,), Compounds of Formula III (e.g., any of 3.1-3.27); a Compound of Formula IV (e.g., any of 4.1-4.22) or a Compound of Formula V or VI in any form, for example free or acid addition salt or prodrug form, or where the compounds contain acidic substituents, in base addition salt form. The Compounds of the Invention are intended for use as pharmaceuticals, therefore pharmaceutically acceptable salts are preferred.

Compounds of the Invention may or may not be used as pharmaceuticals. Therefore, salts which are unsuitable for pharmaceutical uses may be useful, for example, for the isolation or purification of free Compounds of the Invention or their pharmaceutically acceptable salts, and are therefore also included.

As Compounds of Formula II is a subset of Compounds of Formula I, Compounds of Formula II or any of 2.1-2.17 as hereinbefore described may also be useful for the same methods of use, e.g., any of Methods I, I-A to I-E, or II. In addition, the invention also encompasses use of a Compound of Formula II or any of 2.1-2.17, a Compound of Formula III, e.g., any of formulae 3.1-3.27, a Compound of Formula IV, e.g., any of formulae 4.1-4.22, or a Compound of Formula V or VI, as hereinbefore described in the manufacture of a medicament for the treatment or prophylaxis of an infection as hereinbefore described in Method I, I-A to I-E, I(a)-A to I(a)-E, I(b)-A to I(b)-E, I(c)-A to I(c)-E, or II (e.g., bacterial or fungal infection) or II(a), II(b) or II(c). In still another embodiment, the invention also encompasses a pharmaceutical composition comprising a Compound of Formula II or any of 2.1-2.17 as hereinbefore described, in free, pharmaceutically acceptable salt or prodrug form in an admixture with a pharmaceutically acceptable diluent or carrier.

The methods (Method I, I-A through I-E, I(a)-A through I(a)-E, I(b)-A through I(b)-E, I(c)-A through I(c)-E, Method II, II(a), II(b) and II(c)), use, pharmaceutical composition and riboswitch ligands of the current invention are intended to encompass all of the compounds of the invention, which includes any compounds of formula I, e.g, any of 1.1-1.212 or any of formulae I(i)-I(v), e.g., any of 1.213-1.265, formula II, e.g., 2.1-2.17, Formula III, e.g., any of 3.1-3.27, Formula IV, e.g., any of 4.1-4.22, or Formula IV or V, in free, salt or prodrug form. In one preferred embodiment, the compound is selected from any of those set forth in formula 1.261 or 1.262, in free, salt or prodrug form. In another preferred embodiment, the compound of Formula III is a compound selected from any one of those set forth in formula 3.21 or 3.22, in free, salt or prodrug form. In still another preferred embodiment, the compound of Formula IV is a compound selected from any one of those set forth in formula 4.10, 4.12 or 4.21, in free, salt or prodrug form.

The Compounds of the Invention may comprise one or more chiral carbon atoms. The compounds thus exist in individual isomeric, e.g., enantiomeric or diasteriomeric form or as mixtures of individual forms, e.g., racemic/diastereomeric mixtures. Any isomer may be present in which the asymmetric center is in the (R)-, (S)-, or (R,S)-configuration. The invention is to be understood as embracing both individual optically active isomers as well as mixtures (e.g., racemic/diasteromeric mixtures) thereof. Accordingly, the Compound of the Invention may be predominantly, e.g., in pure, or substantially pure, isomeric form, e.g., greater than 70% enantiomeric excess (“ee”), preferably greater than 80% ee, more preferably greater than 90% ee, most preferably greater than 95% ee. The purification of said isomers and the separation of said isomeric mixtures may be accomplished by standard techniques known in the art. Wherein R₃ is -2,3,4,5-tetrahydroxypentyl or 2,3,4-trihydroxypentyl-OP(O)(OH)₂ or 2,3,4-trihydroxypentyl-P(O)(OH)₂, the (2S,3S,4R) configuration is preferred. Therefore, in a particular embodiment of the invention, a compound of Formula I wherein R₃ is -2,3,4,5-tetrahydroxypentyl, the compound is predominantly pure in the (2S,3S,4R) form.

Geometric isomers by nature of substituents about a double bond or a ring may be present in cis (═Z) or trans (=E-) form, and both isomeric forms are encompassed within the scope of this invention.

Compounds of the Invention may in some cases also exist in prodrug form. The term “prodrug” is an art recognized term and refers to a drug precursors prior to administration, but generate or release the active metabolite in vivo following administration, via some chemical or physiological process. For example, when the Compounds of the Invention (e.g., Formula I, e.g., 1.1-1.212 or any of formulae I(i)-I(v), e.g., any of 1.213-1.265, as described in Method I or II, a Compound of Formula II, e.g., 2.1-2.17, a Compound of Formula III, e.g., any of 3.1-3.27, or Formula IV, e.g., any of 4.1-4.22) contain carboxy, phosphate or phosphonate substituents, these substituents may be esterified to form physiologically hydrolysable and acceptable esters (e.g., carboxylic acid, phosphate or phosphonate esters, e.g., —C(O)OR₉, —OP(O)(OR₉)(OR₁₇), —OP(O)(OR₉)(NR₁₃R₁₄)), —P(O)(OR₉)(OR₁₇), —P(O)(OR₉)(NR₁₃R₁₄)). As used herein, “physiologically hydrolysable and acceptable esters” means esters of Compounds of the Present Invention which are hydrolysable under physiological conditions to yield acids, e.g., carboxylic acid, phosphonic or phosphoric acid (in the case of Compounds of the Invention which have carboxy, phosphonate or phosphate substituents) on the one hand and HOR₉ or HOR₁₇ on the other hand, which are themselves physiologically tolerable at doses to be administered. Similarly, the invention encompasses a Compound of the Invention in, e.g., —OP(O)(OR₉)(NR₁₃R₁₄), —OP(O)(NR₁₃R₁₄)(NR₁₅R₁₆), —P(O)(OR₉)(NR₁₃R₁₄) or —P(O)(NR₁₃R₁₄)(NR₁₅R₁₆) prodrug form, wherein the phosphoramidates and phosphonamidates are hydrolysed so as to release the phosphoric or phosphonic acid. In still another embodiment, the invention encompasses a Compound of the Invention which contains an alcohol substituent, e.g., R₃ is hydroxyC₁₋₈alkyl, wherein said compound is the prodrug and is phosphorylated in vivo, e.g., by a kinase to form an active phosphate derivative. As will be appreciated the term thus embraces conventional pharmaceutical prodrug forms.

### Methods of Making Compounds of the Invention

The compounds of the Formula I, II, III, IV, V and VI and their salts may be made using the methods as described and exemplified herein and by methods similar thereto and by methods known in the chemical art. Such methods include, but not limited to, those described below. In the description of the synthetic methods described herein, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. Therefore, at times, the reaction may require to be run at elevated temperature or for a longer or shorter period of time. It is understood by one skilled in the art of organic synthesis that functionality present on various portions of the molecule must be compatible with the reagents and reactions proposed. If not commercially available, starting materials for these processes may be made by procedures, which are selected from the chemical art using techniques which are similar or analogous to the synthesis of known compounds. All references cited herein are hereby incorporated in their entirety by reference.

The synthetic methods for the Compounds of the Present Invention are illustrated below. The significances for the R groups are as set forth above for Formula I or II, unless otherwise indicated. In another embodiment, the significances of the substituents are as set forth in Formula III-VI unless otherwise indicated.

Compounds of Formula I or II may be prepared by reacting Int-4 with alloxan (i.e., pyrimidine-2,4,5,6(1H,3H)-tetrone), in the presence boron oxide and acid, e.g., acetic acid.

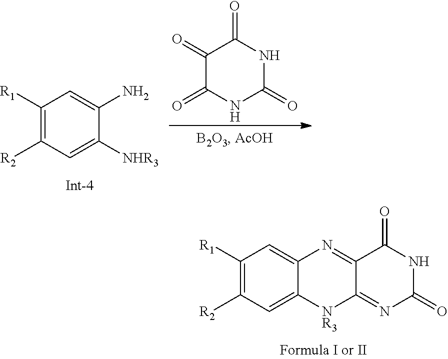

Alternatively, a compound of formula I or II may be prepared comprising the step of reacting Int-4A as described below with a violuric acid at elevated temperature (e.g. greater than 25° C. e.g. about 95° C.

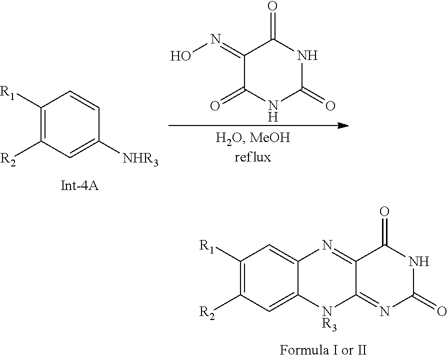

Wherein R₂ of Compound of Formula I or II is —N(R₄)(R₅), (e.g., methylamino, dimethylamino or other amino derivative), said compounds may also be prepared by reacting Int-5 with R₂—H wherein R₂ is —N(R₄)(R₅). This reaction may require heating, e.g., greater than 30° C., e.g., 30°-153° C., e.g., 100° C.

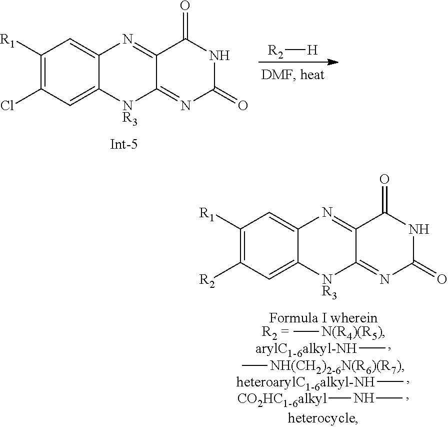

In another embodiment, Compounds of Formula I or II may also be prepared by further comprising the step of reducing, prior to the preparation of Int-5, the nitro group of Int-3 to an amine of Int-4 by using metal (e.g., zinc, tin, iron or sodium borohydride) and acid (e.g., hydrochloric acid). For example, a Compound of Formula I or II may be prepared by further comprising the step of reacting Int-3 with zinc and ammonia in a solvent such as water and ethanol.

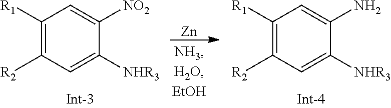

In a further embodiment, Compounds of Formula I may be prepared by further comprising, prior to the preparation of Int-3, the step of reacting Int-2 with a primary amine, R₃NH₂ in the presence of a base such as triethylamine.

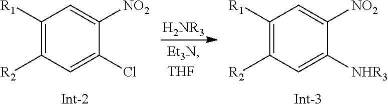

Alternatively, Int-4 wherein R₃ is (2S,3S,4R)-2,3,4,5-tetrahydroxypentyl may be prepared by reacting 3-chloro-4-methyl-phenylamine with acetic anhydride, nitrating the ring using nitric acid in the presence of acetic acid and then reacting the resulting product with a strong base such as sodium methoxide to obtain 5-chloro-4-methyl-2-nitro-phenylamine (Int-3A). Int-3A is then reacted with D-ribose in the presence of ammonium chloride to obtain Int-3B wherein R₃ is ribose. The ribose may undergo ring opening and the nitro group may be reduced to an amine using sodium borohydride and palladium on carbon to yield Int-4.

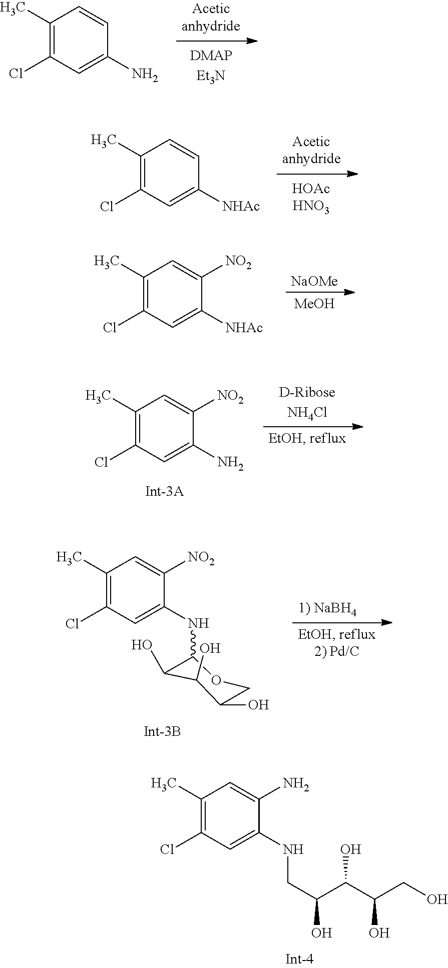

In still a further embodiment, Compounds of Formula I or II may be prepared, by further comprising the step of nitrating, prior to the preparation of Int-2, Intermediate-1 (Int-1), e.g., with sodium nitrate in the presence of an acid, e.g., mineral acid, e.g., sulfuric acid.

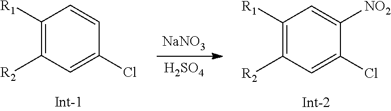

Phosphate derivatives of the Compounds of the Invention may be prepared by reacting a compound of formula I, wherein R₃ is C₁₋₆ alkyl substituted with hydroxy, e.g., R₃ is (2S,3S,4R)-2,3,4,5-tetrahydroxypentyl, with dichlorophosphoric acid.

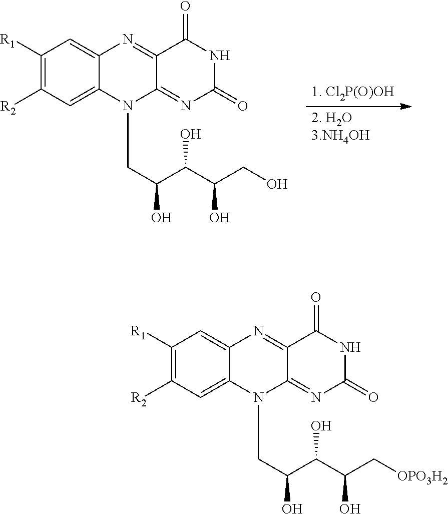

In a particular embodiment, Compounds of formula I or II, e.g., wherein R₁ is methyl, R₂ is dimethylamine and R₃ is -nC₄H₉, —(CH₂)₂OH, —(CH₂)₃OH, —(CH₂)₄OH or —(CH₂)₅OH may be prepared by (1) nitrating 2,4-dichloro-1-methylbenzene using sodium nitrate in the presence of sulfuric acid to yield 1,5-dichloro-2-methyl-4-nitrobenzene, which is then (2) reacted with R₃NH₂ in the presence of a base to yield 5-chloro-4-methyl-2-nitroaniline. This product is then (3) reacted with zinc and ammonium hydroxide in water/ethanol solvent to yield 4-chloro-5-methylbenzene-1,2-diamine, which is then (4) reacted with alloxan in the presence of borane oxide and acetic acid to obtain Int-5. Finally, a Compound of formula I or II as herein described is obtained by (5) reacting Int-5 with dimethylamine in DMF at 100° C.

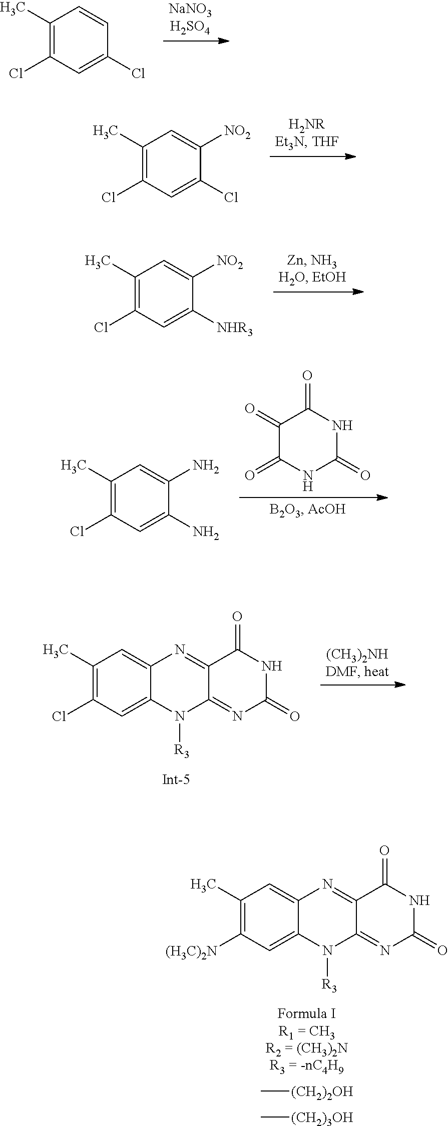

A Compound of Formula I or II having various R₂ substituents may be prepared by starting with a Compound of Formula I, wherein R₂ is halo (e.g., chloro) and reacting such compound with HN(R₄)(R₅).

In a particular embodiment, Compounds of formula I wherein R₃ is a dihydrogen phosphate alkyl may be prepared by reacting Int-4B with diethyl bromoalkylphosphonate (e.g., diethyl 6-bromohexylphosphonate) to yield Int-4A. Int-4A may be converted to Compound of Formula I or II, wherein R₃ is alkylphosphonate dialkyl ester by reacting with violuric acid. The phosphonate ester may be hydrolyzed by using an acid, e.g, hydrochloric acid.

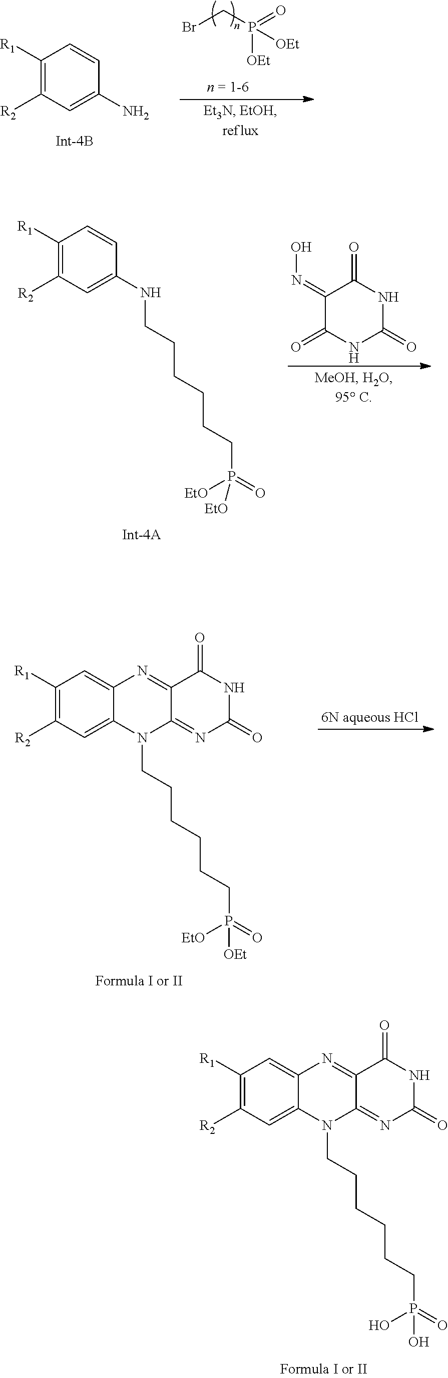

In a particular embodiment, Compounds of formula I, e.g., wherein R₃ is an alkyl amino-alkyltrifluorornethanesulfonamide (e.g. N-(3-(2-(7,8-dimethyl-2,4-dioxo-3,4-dihydrobenzo[g]pteridin-10(2H)-yl)ethylamino)propyl)-1,1,1-trifluoromethanesulfonamide) may be prepared by starting with a Compound of Formula I wherein R₃ is (2R,3S,4S)-2,3,4,5-tetrahydroxypentyl and reacting it with orthoperiodic acid and sulfuric acid to yield Int-6. Int-6 may then be converted to a Compound of Formula I or II, wherein R₃ is an alkyl amino-alkyltrifluoromethanesulfonamide, by reductive amination reaction, e.g., reacting Int-6 with N-(3-aminoalkyl)-trifluoromethanesulfonamide followed by a reducing agent, e.g., sodium cyanoborohydride to yield a sulfonamide derivative of a Compound of Formula I.

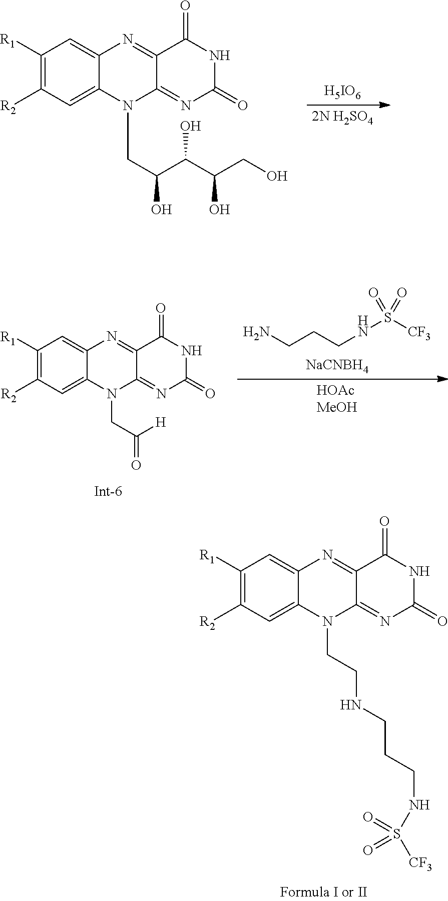

Similarly, Compounds of formula I, e.g., wherein R₃ is benzoic acid aminoalkyl (e.g., 3-(2-(7,8-dimethyl-2,4-dioxo-3,4-dihydrobenzo[g]pteridin-10(2H)-yl)ethylamino)benzoic acid) may be prepared by subjecting Int-6 to a reductive amination reaction as described above by reacting Int-6 with alkyl 3-aminobenzoate (e.g., t-butyl-3-aminobenzoate) followed by sodium cyanoborohydride to yield Int-7 below. Int-7 may be hydrolyzed using an acid, e.g., trifluoroacetic acid, to benzoic acid derivative of Formula I below.

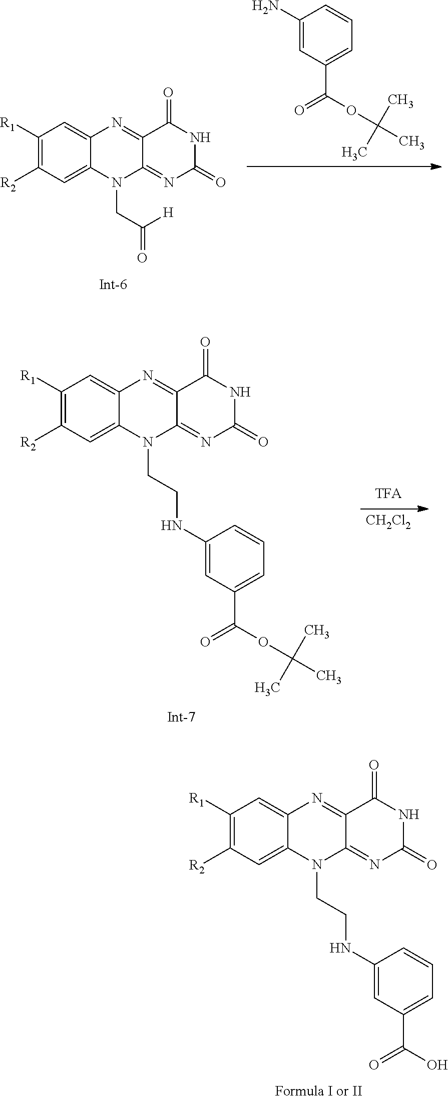

In a particular embodiment, Compounds of formula I, e.g., wherein R₃ is an -alkyl-CONHOH may be prepared by reacting a Compound of Formula I, wherein R₃ is an -alkyl-COOH with an alkyl chloroformate (e.g., isobutylchloroformate) and N-methylmorpholine followed by hydroxylamine hydroxychloride to yield the hydroxamide alkyl derivative.

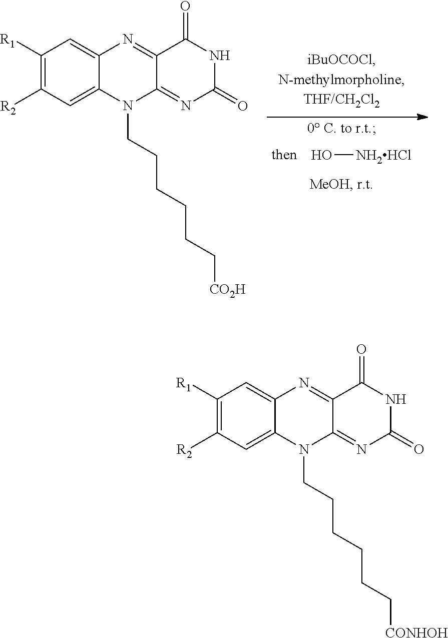

In a particular embodiment, Compounds of formula I wherein R₃ is an alkyl-N(R₆)(R₇) wherein R₆ is -alkyl-CONHOalkyl and R₇ is H may be prepared by protecting the amine of Int-9 with a protecting group, e.g. BOC anhydride to yield Int-10. Int-10 is then coupled with O-benzylhydroxylamine hydrochloride using HBTU and a base, e.g., diisopropylethylamine to yield Int-11, which is then deprotected using an acid, e.g, trifluoroacetic acid.

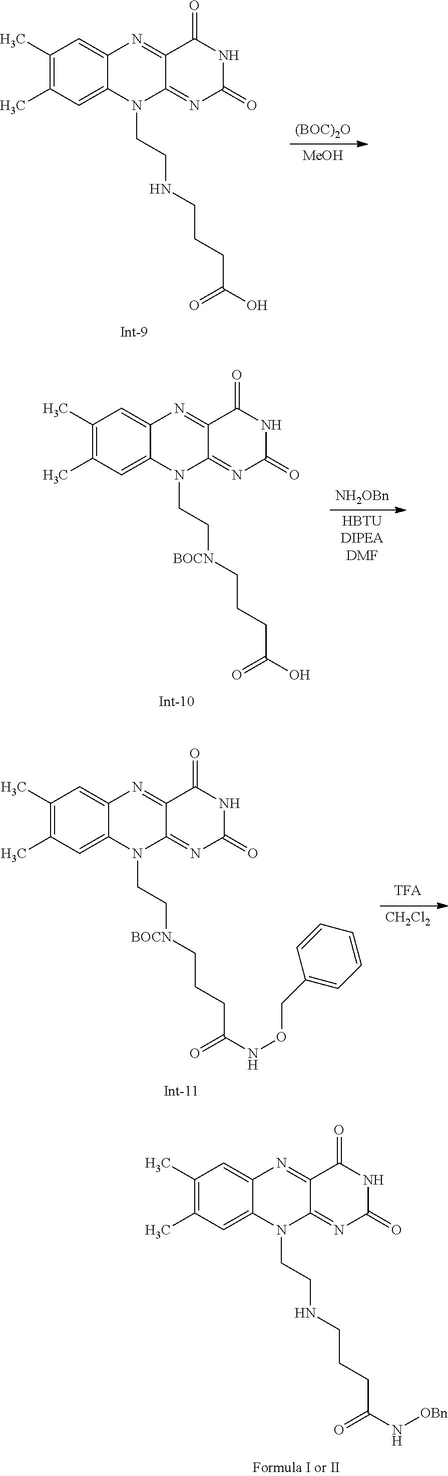

In certain aspect, the invention further provides methods of making the Compounds of the Invention, e.g., as set forth below. Compounds of formula I, wherein R₃ is arylalkylaminoethyl, R₂ is —N(R₄)(R₅) and R₁ is as hereinbefore described in Formula I, I(i)-I(v), II-VI, (e.g., 10-(2-(benzylamino)ethyl)-8-(cyclopropylamino)-7-methylbenzo[g]pteridine-2,4(3H,10H)-dione) may be prepared by subjecting Int-12 to a reductive amination reaction, e.g., by reacting Int-12 with an amine (e.g., phenylmethanamine) followed by a reduction reaction, e.g., using sodium borohydride or cyanoborohydride to yield Int-5 below. Int-5 may be reacted with R₂—H wherein R₂ is —N(R₄)(R₅) to give compounds of formula I below. This reaction may require heating, e.g., greater than 30° C., e.g., 30°-153° C., e.g., 70° C. In-12 may be prepared via oxidative cleavage of 8-chloro-7-methyl-10-((2S,3S,4R)-2,3,4,5-tetrahydroxypentyl)benzo[g]pteridine-2,4(3H,10H)-dione using orthoperiodic acid and sulfuric acid.

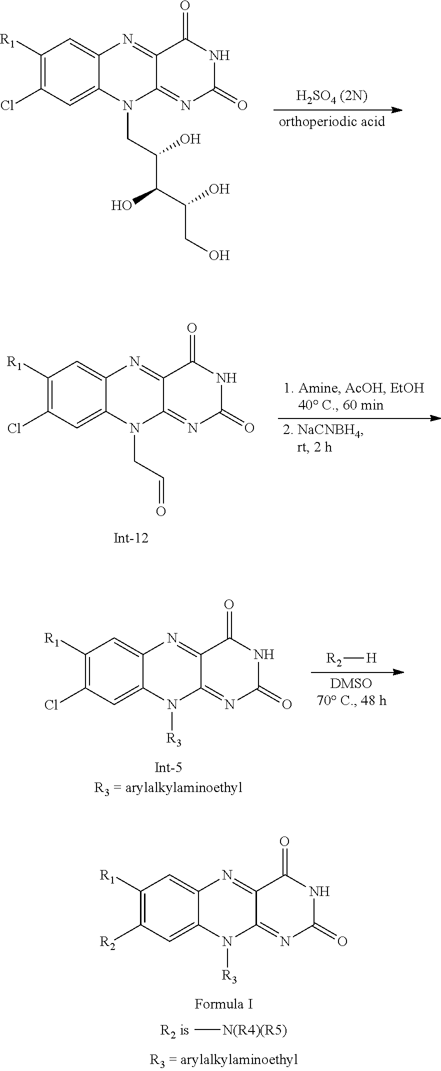

Compounds of formula I, wherein R₃ is arylalkylaminoethyl, R₂ is —O—C₃₋₇cycloalkyl or C₁₋₈alkoxy and R₁ is as hereinbefore described in Formula I, I(i)-I(v), II-VI, (e.g., 10-(2-(benzylamino)ethyl)-8-(cyclopentyloxy)-7-methylbenzo[g]pteridine-2,4(3H,10H)-dione) may also be prepared from an intermediate such as Int-5 in which the amino moiety of the arylalkylaminoethyl moiety is protected (e.g., carbamate group, e.g., BOC,) during the reaction of Int-5 with R₂—H wherein R₂ is —O—C₃₋₇cycloalkyl. The reaction may require base (e.g. sodium hydride) and heating. The protecting group (e.g., carbamate group) may be removed using acid (e.g. HCl) to give compounds of formula I wherein R₃ is arylalkylaminoethyl and R₂ is —O—C₃₋₇cycloalkyl.

Similarly, compounds of formula I, wherein R₃ is 4-(arylalkyl(ethyl)amino)-alkanoic acid, R₂ is —N(R₄)(R₅) and R₁ is as hereinbefore described in Formula I, I(i)-I(v), II-VI, (e.g., 4-(benzyl(2-(8-(cyclopentylamino)-7-methyl-2,4-dioxo-3,4-dihydrobenzo[g]pteridin-10(2H)-yl)ethyl)amino)butanoic acid) may also be prepared from Int-12 by subjecting Int-12 to a reductive amination reaction, e.g., by reacting Int-12 with an appropriate amine (e.g., 4-(benzylamino)butanoic acid) followed by a reduction reaction, e.g., using sodium borohydride or cyanoborohydride to yield Int-5 below (R₃=4-(arylalkyl(ethyl)amino)alkanoic acid). Int-5 may be reacted with R₂—H wherein R₂ is —N(R₄)(R₅) to give compounds of formula I. This reaction may require heating, e.g., greater than 30° C., e.g., 30°-153° C., e.g., 90° C.

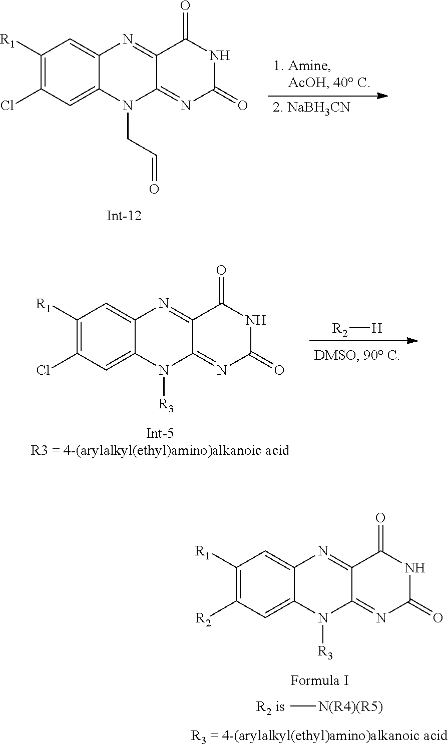

In a particular embodiment, Compounds of formula I wherein R₃ is an alkyl ester or alkyl acid and R₂ is —N(R₄)(R₅) and R₁ is as hereinbefore described in Formula I, I(i)-I(v), II-VI, (e.g., tert-butyl 7-[8-(cyclopropylamino)-7-methyl-2,4-dioxo-3,4-dihydrobenzo[g]pteridin-10(2H)-yl]heptanoate) may be prepared by reacting Int-5 with R₂—H wherein R₂ is —N(R₄)(R₅) to give compounds of formula I, wherein R₁ and R₉ are hereinbefore described. Wherein R₉ is not H (e.g., the compound is an ester), hydrolysis of the ester moiety using base (e.g. lithium hydroxide in THF/water) provides the corresponding acid of formula I below.

Similarly, reaction of Int-5 (R₃=alkyl ester) with R₂—Na wherein R₂ is —O—C₃₋₇cycloalkyl or C₁₋₈alkoxy provides a compound of formula I (e.g., 7-(8-methoxy-7-methyl-2,4-dioxo-3,4-dihydrobenzo[g]pteridin-10(2H)-yl)heptanoic acid) wherein R₂ is —O—C₃₋₇cycloalkyl or C₁₋₈alkoxy and R₃ is an alkyl acid.

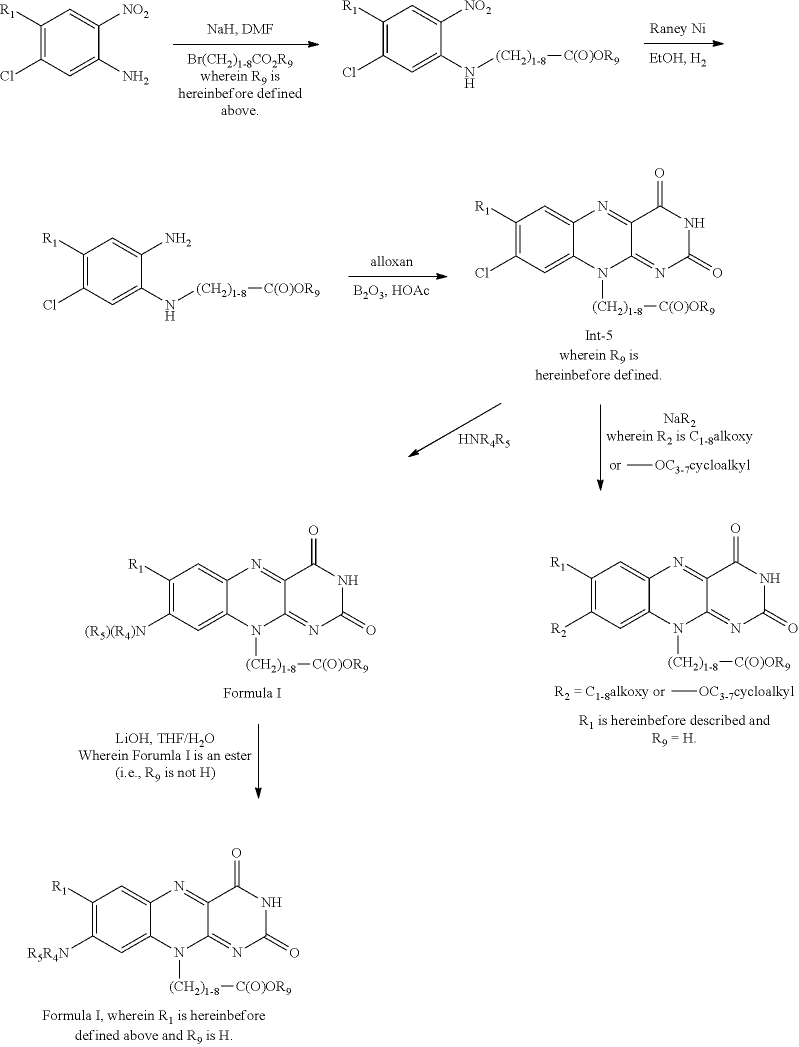

Compounds of Formula I wherein R₃ is C₁₋₈alkyl substituted with —P(O)(OR₉)(NR₁₃R₁₄), —OP(O)(NR₁₃R₁₄)(NR₁₅R₁₆), —P(O)(OR₉)(NR₁₃R₁₄), or —P(O)(NR₁₃R₁₄)(NR₁₅R₁₆) may be prepared by methods known in the art. One method of preparing such phosphoramidates of Formula I is to, e.g., react a compound of Formula I containing a hydroxyl substitutent, e.g., Formula I wherein R₃ is C₁₋₈alkyl substituted with —OH, with Cl—P(O)(OR₉)(NR₁₃R₁₄), Cl—P(O)(NR₁₃R₁₄)(NR₁₅R₁₆), e.g., phenyl methoxyalininyl phosphorocloridates, in the presence of a base, e.g., an amine base, e.g., N-methyl-imidazole, e.g., in a solvent such as dichloromethane or tetrahydrofuran. Preparation of Cl—P(O)(OR₉)(NR₁₃R₁₄), Cl—P(O)(NR₁₃R₁₄)(NR₁₅R₁₆), e.g., phenyl methoxyalininyl phosphorocloridates, as well as other phosphoramidates, bis-phosphoramidates and phosphonamidates may be prepared by using similar methods as those described in McGuigan et al., Antiviral Res. (1992) 17:311-321, McGuigan et al., Antiviral Res. (1991) 15:255-263, Serafinowska et al., J. Med. Chem. (1995) 38:1372-1379, Mehellou et al., Bioorg. Med. Chem. Lett. (2007) 17:3666-3669, Jones et al. (1991) 2:35-39 and U.S. Pat. No. 7,071,176, the contents of each of which are herein incorporated by reference in their entirety. Bis(phosphonamidates) of Compounds of Formula I can also be prepared by using methods similar to those described in WO 2006/023515 (the contents of which are incorporated by reference in their entirety), for example, activating the phosphonic acid substituent of a Compound of Formula I (e.g., wherein R₃ is alkyl substituted with —P(O)(OH)₂) with e.g., oxalyl chloride, and reacting the resulting compound with HN(R₁₃R₁₄)(R₁₅R₁₆), e.g., methylalanine or methylalanine ethyl ester, in the presence of a base, e.g., an amine base, e.g., diethylisopropylamine or diisopropylethylamine, triethylamine or the like.

### Methods of Using Compounds of the Invention

The Compounds of the Present Invention are useful for the treatment of an infection, particularly an infection by bacteria including but not limited to Moraxella catarrhalis, Klebsiella pneumoniae, Staphylococcus epidermidis, Streptococcus viridians, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenza, Listeria monocytogenes, Salmonella enterica, Vibrio choierae, Enterococcus faecalis and Yersinia pestis. In addition to these bacteria, The Compounds of the Present Invention are useful for the treatment of an infection, particularly an infection by bacteria including but not limited to Bacillus subtilis, Streptococcus pyogenes and/or Borrelia burgdorferi bacteria. In a preferred embodiment, the bacteria is selected from any one of the following: Staphylococcus aureus, Staphylococcus epidermidis, Bacillus subtilis, Enterococcus faecalis, Streptococcus pneumoniae, Streptococcus pyogenes, Escherichia coli, Pseudomonas aeruginosa, Klebsiella pneumoniae, Haemophilus influenzae, Acinetobacter baumannii. In another preferred embodiment, the infection is by the Staphylococcus aureus and/or Staphylococcus epidermidis bacteria.

The invention therefore provides methods of treatment of any one or more of the following conditions: anthrax infection, staphylococcal scalded skin syndrome (staph infections), pneumonia, impetigo, boils, cellulitis folliculitis, furuncles, carbuncles, scalded skin syndrome, abscesses, meningitis, osteomyelitis endocarditis, Toxic Shock Syndrome (TSS), septicemia, acute sinusitis, otitis media, septic arthritis, endocarditis, peritonitis, pericarditis, cellulitis, brain abscess, tularemia, urinary tract infection, empyema, food poisoning, diarrhea and conjunctivitis; comprising administering an effective amount of a Compound of Formula I, e.g., any of 1.1-1.212, or any of formulae I(i)-I(v), e.g., any of 1.213-1.265, preferably formula 1.261 or 1.262, as described in Method I, or formula II, in free, pharmaceutically acceptable salt or prodrug form, to a human or animal patient in need thereof. In other embodiments, the invention provides methods of treatment of the conditions set forth above comprising administering an effective amount of a Compound of Formula III, e.g., any of 3.1-3.27; a Compound of Formula IV, e.g., any of 4.1-4.22, a Compound of Formula V or VI, in free, pharmaceutically acceptable salt or prodrug form. The words “treatment” and “treating” are to be understood accordingly as embracing prophylaxis and treatment or amelioration of symptoms of disease as well as treatment of the cause of the disease.

The term “patient” as used herein encompasses human or non-human (e.g., animal).

Dosages employed in practicing the present invention will of course vary depending, e.g. on the particular disease or condition to be treated, the particular Compound of the Invention used, the mode of administration, and the therapy desired. Administration of a therapeutically active amount of the therapeutic compositions is defined as an amount effective, at dosages and for periods of time necessary to achieve the desired result. For example, a therapeutically effective amount of a Compound of the Present Invention reactive with at least a portion of FMN riboswitch may vary according to factors such as the disease state, age, sex, and weight of the individual, and the ability of the compound to elicit a desired response in the individual. Dosage regiment may be adjusted to provide the optimum therapeutic response. For example, several divided doses may be administered daily or the dose may be proportionally reduced as indicated by the exigencies of the therapeutic situation.

Pharmaceutical compositions comprising Compounds of the Present Invention may be prepared using conventional diluents or excipients and techniques known in the galenic art. Thus oral dosage forms may include tablets, capsules, solutions, suspensions and the like. The term “pharmaceutically acceptable carrier” as used herein is intended to include diluents such as saline and aqueous buffer solutions. The Compounds of the Present Invention may be administered in a convenient manner such as by injection such as subcutaneous, intravenous, by oral administration, inhalation, transdermal application, intravaginal application, topical application, intranasal, sublingual or rectal administration. Depending on the route of administration, the active compound may be coated in a material to protect the compound from the degradation by enzymes, acids and other natural conditions that may inactivate the compound. In a preferred embodiment, the compound may be orally administered. In another embodiment, the compound is administered via topical application.

In certain embodiment, the Compounds of the Invention may be administered alone or in conjunction, e.g., at or about the same time, simultaneously and separately or simultaneously in an admixture, with another agent, e.g., an agent to facilitate entry or permeability of the Compounds of the Invention into the cell, e.g., an antimicrobial cationic peptide. Antimicrobial cationic peptides include peptides which contain (1) a disulfide-bonded β-sheet peptides; (2) amphipathic α-helical peptides; (3) extended peptides; or (4) loop-structured peptides. Examples of cationic peptide include but are not limited to defensins, cecropins, melittins, magainins, indolicidins, bactenecin and protegrins. Other examples of antimicrobial cationic peptides include but are not limited to human neutrophil defensin-1 (1-INP-1), platelet microbicidal protein-1 (tPMP), inhibitors of DNA gyrase or protein synthesis, CP26, CP29, CP11CN, CP10A, Bac2A-NH₂ as disclosed in Friedrich et al., Antimicrob. Agents Chemother. (2000) 44(8):2086, the contents of which are hereby incorporated by reference in its entirety. Further examples of antibacterial cationic peptides include but are not limited to polymyxin e.g., polymixin B, polymyxin E or polymyxin nonapeptide. Therefore, in another embodiment, the Compounds of the Invention may be administered in conjunction with polymyxin, e.g., polymixin B, polymyxin E or polymyxin nonapeptide, preferably polymyxin B.

In still another embodiment, the Compounds of the Invention may be administered alone or in conjunction, e.g., at or about the same time, simultaneously and separately, or simultaneously in an admixture, with other antimicrobial agents, e.g., other antifungal or other systemic antibacterial (bactericidal or bacteriostatic) agents. Examples of bacterial agents include agents which inhibit bacterial cell wall synthesis (e.g., penicillins, cephalosporins, carbapenems, vancomycin), agents which damage cytoplasmic membrane (e.g., polymixins as discussed above), agents which modify the synthesis or metabolism of nucleic acids (e.g., quinolones, rifampin, nitrofurantoin), agents which inhibit protein synthesis (aminoglycosides, tetracyclines, chloramphenicol, erythomycin, clindamycin), agents which interfer with the folate synthesis (e.g., folate-inhibitors), agents which modify energy metabolism (e.g., sulfonamides, trimethoprim) and/or other antibiotics (beta-lactam antibiotic, beta-lactamase inhibitors). Specific anti-infective agents, particularly antibacterial and antifungal agents, are discussed in Remington: The Science and Practice of Pharmacy, Chapter 90, pp. 1626-1684 (21^{st} Ed., Lippincott Williams & Wilkins 2005), the contents of which are hereby incorporated by reference.

### Binding of Ligand to Riboswitch

### Example 1

An in-line probing assay, as described in Regulski and Breaker, “In-line probing analysis of riboswitches”, (2008), Methods in Molecular Biology, Vol 419, pp 53-67, the contents of which are incorporated by reference in its entirety, is used to estimate the dissociation binding constants for the interaction of each of the ligands described herein with an FMN riboswitch amplified from the genome of Bacillus subtilis. Precursor mRNA leader molecules are prepared by in vitro transcription from templates generated by PCR and [5′-³²P]-labeling using methods described previously (Regulski and Breaker, In-line probing analysis of riboswitches (2008), Methods in Molecular Biology Vol 419, pp 53-67). Approximately 5 nM of labeled RNA precursor is incubated for 41 hours at 25° C. in 20 mM MgCl₂, 50 mM Tris HCl (pH 8.3 at 25° C.) in the presence or absence of increasing concentrations of each ligand. In-line cleavage products are separated on 10% polyacrylamide gel electrophoresis (PAGE), and the resulting gel is visualized using a Molecular Dynamics Phosphorimager. The location of products bands corresponding to cleavage are identified by comparison to a partial digest of the RNA with RNase T1 (G-specific cleavage) or alkali (nonspecific cleavage).

In-line probing exploits the natural ability of RNA to self-cleave at elevated pH and metal ion concentrations (pH≈8.3, 25 mM MgCl₂) in a conformation-dependent manner. For self-cleavage to occur, the 2′-hydroxyl of the ribose must be “in-line” with the phosphate-oxygen bond of the internucleotide linkage, facilitating a S_{N}2P nucleophilic transesterification and strand cleavage. Typically, single-stranded regions of the Riboswitch are dynamic in the absence of an active ligand, and the internucleotide linkages in these regions can frequently access the required in-line conformation. Binding of an active ligand to the Riboswitch generally reduces the dynamics of these regions, thereby reducing the accessibility to the in-line conformation, resulting in fewer in-line cleavage events within those regions. These ligand-dependent changes in RNA cleavage can be readily detected by denaturing gel electrophoresis.

The experiments show that various Compounds of the invention, particularly 1.204, 1.210, 1.261, 1.262, 3.21, 4.10, 4.12 or 4.21, in free, salt or prodrug form, have a binding affinity to FMN riboswitch with an IC₅₀ value of less than, or equal to, 10 μM.

### Minimum Inhibitory Concentration

### Example 1A

The MIC assays are carried out in a final volume of 100 μL in 96-well clear round-bottom plates according to methods established by the Clinical Laboratory Standards Institute (CLSI). Briefly, test compound suspended in 100% DMSO (or another suitable solubilizing buffer) is added to an aliquot of media appropriate for a given pathogen to a total volume of 50 μL. This solution is serially diluted by 2-fold into successive tubes of the same media to give a range of test compound concentrations appropriate to the assay. To each dilution of test compound in media is added 50 μl of a bacterial suspension from an overnight culture growth in media appropriate to a given pathogen. Final bacterial inoculum is approximately 10⁵-10⁶ CFU/well. After growth for 18-24 hours at 37° C., the MIC is defined as the lowest concentration of antimicrobial agent that completely inhibits growth of the organism as detected by the unaided eye, relative to control for bacterial growth in the absence of added antibiotic. Ciprofloxacin is used as an antibiotic-positive control in each screening assay. Each of the bacterial cultures that are available from the American Type Culture Collection (ATCC, www.atcc.org) is identified by its ATCC number.

The experiments show that various compounds of the invention, e.g., various compounds set forth in formula 1.210, any of 1.259-1.264, e.g., 1.263, various compounds of formula III, e.g., various compounds of formula 3.20, various compounds of formulae 4.9-4.20, e.g., 4.11, have a minimum inhibitory concentration (MIC) of less than 128 μg/mL and in some instances, less than 32 μg/mL.

### Synthesis of Flavin Derivatives of the Invention:

Temperatures are given in degrees Celsius (° C.); unless otherwise stated, operations are carried out at room or ambient temperature, that is, at a temperature in the range of 18-25° C. Chromatography means flash chromatography on silica gel; thin layer chromatography (TLC) is carried out on silica gel plates. Samples were dissolved in deuterated solvents for NMR spectroscopy. NMR data is in the delta values of major diagnostic protons, given in parts per million (ppm) relative to the appropriate solvent signals. Conventional abbreviations for signal shape are used. For mass spectra (MS), the lowest mass major ion is reported for molecules where isotope splitting results in multiple mass spectral peaks. Solvent mixture compositions are given as volume percentages or volume ratios. In cases where the NMR spectra are complex, only diagnostic signals are reported. The LC-MS method is as described in Method C of analytical HPLC analysis below.

### General Methods for Analytical HPLC Analysis:

Method A: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: 95% aqueous (0 to 0.5 min); a gradient from 95% aqueous to 98% organic (0.5 to 10.5 min); 98% organic (2 min); a gradient from 98% organic to 95% aqueous (5.5 min); 95% aqueous (1 min).
Method B: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: 95% aqueous (0 to 0.5 min); a gradient from 95% aqueous to 100% organic (0.5 to 10.5 min); a gradient from 100% organic to 95% aqueous (2 min); 95% aqueous (4 min).
Method C: Analytical LCMS is performed using a YMC Combiscreen ODS-AQ, 5 μm, 4.6×50 mm column. The aqueous phase is 1% 2 mM NH₄OAc in 90:10 IPA:H₂O, 0.03% TFA in USP water. The organic phase is 1% 2 mM NH₄OAc in 90:10 IPA:H₂O, 0.03% TFA in acetonitrile. The elution profile is as follows: a gradient from 95% aqueous to 100% organic (0 to 10 min); 100% organic (2 min); a gradient from 100% organic to 95% aqueous (0.1 min); 95% aqueous (3 min).
Method D: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: a gradient from 95% aqueous to 75% aqueous (0 to 10 min); a second gradient from 75% aqueous to 98% organic (2.5 min); a third gradient to 95% aqueous (over 1 min).
Method E: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: a gradient from 95% aqueous to 40% aqueous (0 to 10 min); a second gradient from 40% aqueous to 2% aqueous (2 min); 2% aqueous (1 min); 2% aqueous to 95% aqueous (4 min).
Method F: Analytical HPLC is performed using a Luna Prep C₁₈, 100 Å 5 μm, 4.6×100 mm column. The aqueous phase is 0.1% TFA in USP water. The organic phase is 0.1% TFA in acetonitrile. The elution profile is as follows: a gradient from 95% aqueous to 60% aqueous (0 to 10 min); a second gradient from 60% aqueous to 2% aqueous (2 min); 2% aqueous (1 min); 2% aqueous to 95% aqueous (4 min).
System A: Agilent 1100 HPLC, Agilent Scalar C18 150×4.6 mm 5 micron column, 1.5 mL/min, Solvent A—Water (0.1% TFA), Solvent B—Acetonitrile (0.07% TFA), Gradient—10 min 95% A to 95% B; 5 min hold; then recycle, UV Detection @ 214 and 250 nm.
System B: Agilent 1100 HPLC, Agilent XDB C8 150×4.6 mm 5 micron column, 1.5 mL/min, Solvent A—Water (0.1% TFA), Solvent B—Acetonitrile (0.07% TFA), Gradient—10 min 95% A to 95% B; 5 min hold; then recycle, UV Detection @ 214 and 250 nm.
System C: Agilent 1100 HPLC, Agilent XDB C18 50×4.6 mm 1.8 micron column, 1.5 mL/min, Solvent A—Water (0.1% TFA), Solvent B—Acetonitrile (0.07% TFA), Gradient—7 min 95% A to 95% B; 1 min hold; then recycle, UV Detection @ 214 and 254 nm.
System D: Agilent 1100 HPLC, Agilent XDB C18 50×4.6 mm 1.8 micron column, 1.5 mL/min, Solvent A—Water (0.1% TFA), Solvent B—Acetonitrile (0.07% TFA), Gradient—5 min 95% A to 95% B; 1 min hold; then recycle, UV Detection @ 214 and 254 nm.

### General Procedure for Preparative HPLC Conditions.

Method 1: Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: 100% aqueous (0 to 3 min); a gradient from 100% aqueous to 98% organic (3 to 21 min); 98% organic (1 min); a gradient from 98% organic to 95% aqueous (1 min); 95% aqueous (1 min).
Method 2: Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: a gradient from 95% aqueous to 25% organic (0 to 10 min); a second gradient from 25% organic to 98% organic (over 2.5 min min); a third gradient to 95% aqueous (over 1 min).
Method 3: Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: isocratic conditions of 20% organic (0-3 min); a gradient from 80% aqueous to 50% organic (3 to 16 min); isocratic conditions of 50% organic (16 to 18 min); a second gradient from 50% organic to 70% organic (from 18 to 25 min); a third gradient from 70% organic to 100% organic (over 0.5 min); then isocratic conditions of 100% organic (over 1 min); a fourth gradient from 100% organic to 95% aqueous (over 1 min.).
Method 4: A SunFire™ Prep C18 OBD™ 5 μm, 30×100 mm column. The aqueous phase is 0.1% TFA in USP water. The organic phase is acetonitrile. The elution profile is as follows: a gradient from 100% aqueous to 60% organic (0 to 29 min); then to 98% organic (29 to 31 min); 98% organic (2 min); a gradient from 98% organic to 100% aqueous (2 min); 100% aqueous (2 min).
Method 5: Preparatory HPLC is performed using a SunFire™ Prep C18 OBD™ 5 μm, 30×100 mm column. The aqueous phase is 0.1% TFA in USP water. The organic phase is acetonitrile. The elution profile is as follows: isocratic conditions of 20% organic (0-3 min); a gradient from 80% aqueous to 50% organic (3 to 16 min); isocratic conditions of 50% organic (16 to 18 min); a second gradient from 50% organic to 70% organic (from 18 to 25 min); a third gradient from 70% organic to 100% organic (over 0.5 min); then isocratic conditions of 100% organic (over 1 min); a fourth gradient from 100% organic to 95% aqueous (over 1 min.)
Method 6: Varian PrepStar, Phenomenex Luna(2) C18 250×21.2 mm 10 micron column, 20 mL/min, Solvent B—Water (0.1% TFA), Solvent A-Acetonitrile (0.07% TFA), Gradient—10 min 5% A to 80% A; 5 min 80% A to 100% A; 5 min hold; then recycle, UV Detection @ 254 nm.

### TERMS AND ABBREVIATIONS

Cat.=catalytic,
CAN=ammonium cerium (IV) nitrate,
CBzCl=benzyl chloroformate
D-ribose=(2R,3R,4R)-2,3,4,5-tetrahydroxypentane,
equiv.=equivalent(s),
h=hour(s),
HATU=2-(1H-7-Azabenzotriazol-1-yl)-1,1,3,3-tetramethyl uronium hexafluorophosphate methanaminium,
HBTU=2-(1H-Benzotriazole-1-yl)-1,1,3,3-tetramethyluronium hexafluorophosphate,
ISCO=normal phase silica gel cartridges supplied by Teledyne ISCO,
Min.=minute(s)
PMB=p-methoxybenzyl,
POMCl=pivaloyloxymethylchloride,
PyBOP=benzotriazol-1-yl-oxytripyrrolidinophosphonium hexafluorophosphate,
rt=room temperature,
RNase T1=an endoribonuclease that specifically degrades single-stranded RNA at G residues,
TBAI=tetrabutylammonium iodide,
TLC=thin layer chromatography,
TMSBr=trimethylsilyl bromide,
Tris HCl=Tris (hydroxymethyl)aminomethane hydrochloride

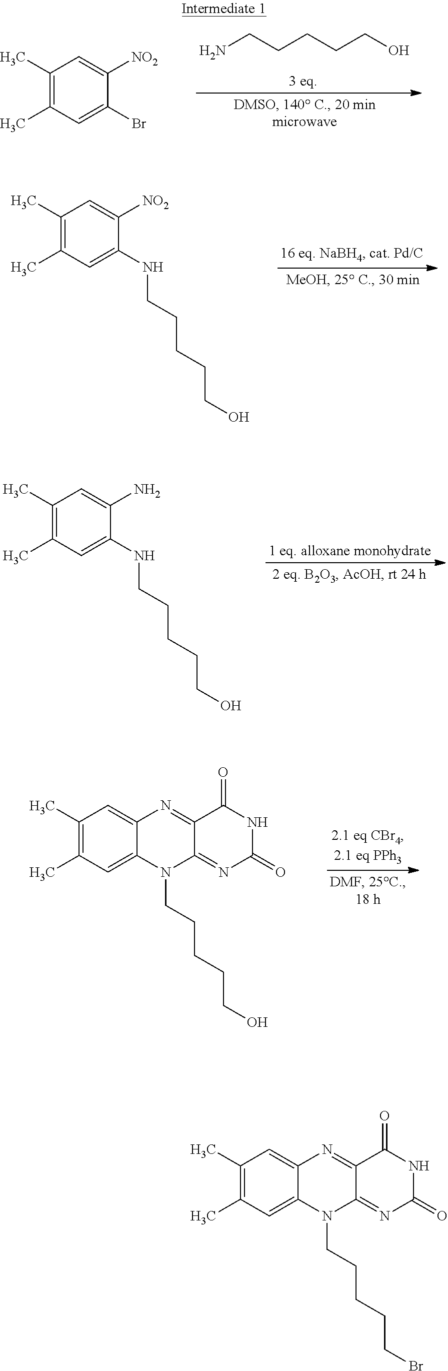

### Preparation of 10-(6-bromopentyl)-7,8-dimethylbenzo[g]pteridine-2,4(3H,10H)-dione

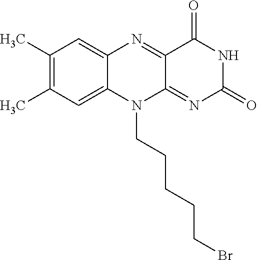

### Step 1 Preparation 5-(4,5-dimethyl-2-nitrophenylamino)pentan-1-ol

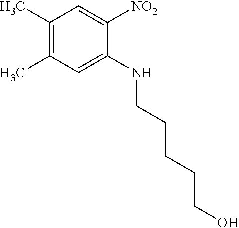

To a solution of 1-bromo-4,5-dimethyl-2-nitrobenzene (200 mg, 0.870 mmol) in anhydrous DMSO (1 mL), is added 5-aminopentan-1-ol (170 mg, 2.608 mmol). The reaction mixture is heated in a microwave at 140° C. for 20 min. The reaction mixture is concentrated under vacuum and diluted with water (5 mL) and the aqueous layer is extracted with DCM (3×5 mL). The organic layer is dried over Na₂SO₄, filtered, and concentrated under reduced pressure. Desired product (147 mg) is isolated (yield: 67%). ¹H NMR (400 MHz, CDCl₃) δ 1.55 (m, 2H), 1.66 (m, 2H), 1.79 (m, 2H), 2.20 (s, 3H), 2.29 (s, 3H), 2.38 (s, 2H), 3.32 (m, 2H), 3.71 (m, 2H), 6.64 (s, 1H), 7.95 (s, 1H).

### Step 2 Preparation of 5-(2-amino-4,5-dimethylphenylamino)pentan-1-ol

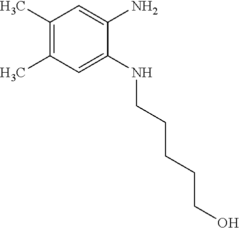

To a solution of 5-(4,5-dimethyl-2-nitrophenylamino)pentan-1-ol (147 mg, 0.583 mmol) in anhydrous MeOH (6 mL) under argon, is added Pd/C (8.4 mg) and sodium borohydride (64 mg, 1.68 mmol), the hydrogen atmosphere is retained with a balloon and the reaction mixture is stirred at room temperature for 30 min. The reaction mixture is filtered through celite, which is washed liberally with EtOH, and the solution is then concentrated to obtain the crude product as a clear, colourless oil which is used in the next step.

### Step 3 Preparation of 10-(5-hydroxypentyl)-7,8-dimethylbenzo[g]pteridine-2,4(3H,10H)-dione

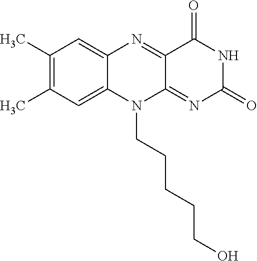

Crude 5-(2-amino-4,5-dimethylphenylamino)pentan-1-ol (0.583 mmol) is dissolved in glacial acetic acid (13 mL) under argon. Alloxan monohydrate (94 mg, 0.583 mmol) and boron oxide (81 mg, 1.165 mmol) are added to the stirring solution and the reaction is maintained under an argon atmosphere at 25° C. with stirring for 2 h. The reaction mixture is evaporated under vacuum and the residue is dry loaded on silica gel using DCM as a solvent and purified by BIOTAGE flash column chromatography using a gradient from 0 to 10% MeOH in DCM as eluent. Desired product (70 mg) is isolated (yield: 37%). ¹H NMR (400 MHz, DMSO) δ 1.48 (m, 4H), 1.70 (m, 2H), 2.38 (s, 3H), 2.49 (s, 3H), 3.40 (m, 2H), 4.40 (t, 1H), 4.55 (m, 2H), 7.78, (s, 1H), 7.88 (s, 1H), 11.28 (s, 1H). ESI(+) [M+Na]⁺=351.2.

### Step 4 Preparation of 10-(5-bromopentyl)-7,8-dimethylbenzo[g]pteridine-2,4(3H,10H)-dione

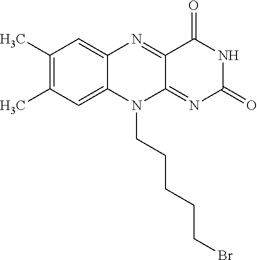

To a solution of 10-(5-hydroxypentyl)-7,8-dimethylbenzo[g]pteridine-2,4(3H,10H)-dione (72 mg, 0.219 mmol) and carbon tetrabromide (80 mg, 0.241 mmol) in anhydrous DMF (5 mL) at 0° C., is added triphenyl phosphine (152 mg, 0.460 mmol) portion-wise. The reaction mixture is stirred at room temperature for 18 h. The reaction mixture is concentrated under reduced pressure and the residue is dry loaded on silica gel using DCM:MeOH (50:50) as a solvent and purified by BIOTAGE flash column chromatography using a gradient from 0 to 2% MeOH in DCM as eluent. Desired product (60 mg) is isolated (yield: 70%). ¹H NMR (400 MHz, DMSO) δ 1.60 (m, 2H), 1.73 (m, 2H), 1.90 (m, 2H), 2.40 (s, 3H), 2.50 (s, 3H), 3.58 (t, 2H), 4.59 (m, 2H), 7.80 (s, 1H), 7.90 (s, 1H), 11.31 (s, 1H). ESI(+) m/z=391.1, 393.1.

### Example 2

### 12R,3S,4S)-5-(8-(dimethylamino)-7-methyl-2,4-dioxo-3,4-dihydrobenzo[g]pteridin-10(2H)-yl)-2,3,4-trihydroxypentyl dihydrogen phosphate

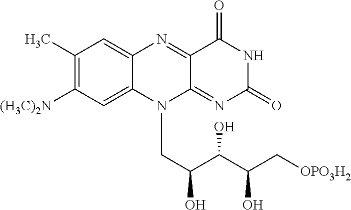

### Step 1 Preparation of (2R,3S,4S)-5-(8-(dimethylamino)-7-methyl-2,4-dioxo-3,4-dihydrobenzo[g]pteridin-10(2H)-14)-2,3,4-trihydroxypentyl dihydrogen phosphate

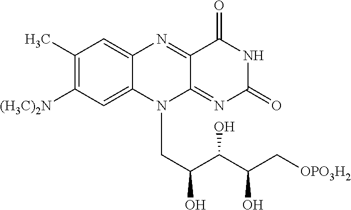

To roseoflavin (40 mg, 0.099 mmol) is added 0.3 mL of a solution of dichlorophosphoric acid (prepared by dropping water (7.2 mL, 400 mmol) over the course of 12 h to a flask containing phosphorus oxychloride (36.6 mL, 400 mmol), cooled to 0° C.). The mixture is stirred at room temperature for 4 h, at which point water (1 mL) is added. The solution quickly becomes warm. The solution is allowed to cool to room temperature, at which point it is neutralized with aqueous ammonium hydroxide. The reaction mixture is purified by preparative HPLC (Method 1). The desired product (23 mg) is isolated following lyophilization (Yield: 47.9%). ¹H NMR (400 MHz, D₂O) δ 2.39 (s, 3H), 3.17 (s, 6H), 3.97 (m, 3H), 4.33 (m, 2H), 4.86 (m, 2H), 6.66 (s, 1H), 7.35 (s, 1H); ESI(−) m/z=484.1.

### Example 3

### Phosphoric acid mono-[5-(8-dimethylamino-7-methyl-2,4-dioxo-3,4-dihydro-2H-benzo[g]pteridin-10-yl)-pentyl]ester

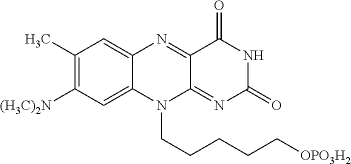

### Step 1 Preparation of Phosphoric acid 5-(8-dimethylamino-7-methyl-2,4-dioxo-3,4-dihydro-2H-benzo[g]pteridin-10-yl)-pentyl ester dimethyl ester

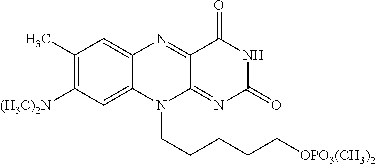

8-Dimethylamino-10-(5-hydroxy-pentyl)-7-methyl-10H-benzo[g]pteridine-2,4-dione (100 mg, 0.28 mmol) (prepared using the procedure described in example 10 steps 2-5 using 5-aminopentan-1-ol instead of butyl amine in step 2) is dissolved in dimethylchlorophosphate (2 mL), and the mixture heated to 50° C. while stirring. After 2 h, the reaction mixture is cooled to room temperature, and volatiles are removed under reduced pressure. The resulting residue is purified by preparative HPLC (Method 1). Phosphoric acid 5-(8-dimethylamino-7-methyl-2,4-dioxo-3,4-dihydro-2,4-benzo[g]pteridin-10-yl)-pentyl ester dimethyl ester (30 mg) is isolated (Yield: 23.0%). LC-MS m/z 466.2 [M+H], retention time 3.00 min.

### Step 2 Preparation of Phosphoric acid mono-[5-(8-dimethylamino-7-methyl-2,4-dioxo-3,4-dihydro-2H-benzo[g]pteridin-10-yl)-pentyl]ester

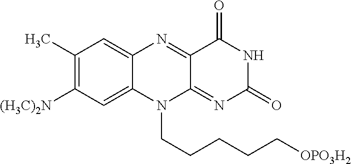

Phosphoric acid 5-(8-dimethylamino-7-methyl-2,4-dioxo-3,4-dihydro-2H-benzo[g]pteridin-10-yl)-pentyl ester dimethyl ester (30 mg, 0.064 mmol) is dissolved in anhydrous acetonitrile (15 mL), and the mixture is stirred at room temperature. Trimethylsilylbromide (80 μL, 0.60 mmol) is added dropwise to the stirring solution, and the reaction is stirred for 3 h at room temperature. The solution is concentrated under reduced pressure and the resulting residue is dissolved in 95:5 methanol:water (15 mL) and stirred at room temperature. After 1 h, 1.0 N aqueous HCl (5 mL) is added to the reaction, which is then stirred at room temperature for 12 h. The reaction mixture is purified by preparative HPLC (Method 1). Phosphoric acid mono-[5-(8-dimethylamino-7-methyl-2,4-dioxo-3,4-dihydro-2H-benzo[g]pteridin-10-yl)-pentyl]ester (14 mg) is isolated (Yield: 50.0%). LC-MS m/z 438.1 [M+H], retention time 2.07 min. ¹H NMR (400 MHz, CD₃OD) δ 1.69 (m, 2H), 1.83 (m, 2H), 1.93 (m, 2H), 2.55 (s, 3H), 3.22 (s, 6H), 4.05 (m, 2H), 4.72 (m, 2H), 6.86 (s, 1H), 7.80 (s, 1H).

### Example 4

### 8-(2-methoxyethylamino)-7-methyl-10-((2S,3S,4R)-2,3,4,5-tetrahydroxypentyl)benzo[g]pteridine-2,4(3H,10H)-dione

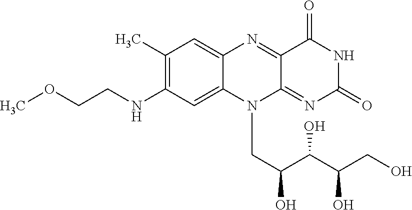

### Step 1 Preparation of N-(3-Chloro-4-methyl-phenyl)-acetamide

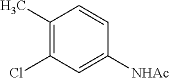

To a solution of 3-chloro-4-methyl-phenylamine (30 mL, 0.25 mol), Et₃N (104 mL, 0.75 mol), and DMAP (50 mg) in 750 mL of ethyl acetate at 0° C., is added acetic anhydride (22.4 ml, 0.237 mol) in 100 mL of ethyl acetate, dropwise over 1 h. Following completion of the addition, the reaction mixture is removed from the ice bath, and then stirred at room temperature for 5 h. The reaction is diluted with 200 mL of water and the organic layer is separated, and washed with brine. The organic layer is dried over Na₂SO₄, filtered, and concentrated under reduced pressure. The crude material is then triturated with hexanes and the solid filtered to yield 38.8 g of the desired product as an off-white solid (Yield: 84%).

### Step 2 Preparation of N-(5-Chloro-4-methyl-2-nitro-phenyl)-acetamide

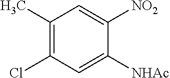

To a solution of N-(3-chloro-4-methyl-phenyl)-acetamide (38.8 g, 0.211 mol), in acetic anhydride (84 mL, 0.89 mol), and acetic acid (37 mL) cooled to −10° C., is added nitric acid (17 mL) in 20 mL of acetic acid, dropwise over 0.5 h. Following completion of the addition, an additional 30 mL of acetic anhydride is added and the reaction mixture is removed from the ice bath, and then stirred at room temperature for 4.5 h. The reaction is poured into 300 mL of cold water and the solid is filtered, washed with water, ethanol, and ethyl ether. The crude is recrystallized from EtOH, filtered, and washed with cold ethanol to yield 23.7 g of the desired product as yellow solid (Yield: 49%).

### Step 3 Preparation of 5-Chloro-4-methyl-2-nitro-phenylamine [Int-3A]

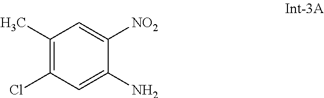

To a solution of N-(5-chloro-4-methyl-2-nitro-phenyl)-acetamide (23.7 g, 0.103 mol) in MeOH (80 mL) at room temperature is added sodium methoxide in methanol (25 weight percent, 20 mL). An additional 60 mL of MeOH is added for solubility and the solution is stirred for 5 h. The reaction is diluted with 500 mL of water and the solid is filtered, washed with water, isopropanol, and hexanes to yield 19.8 g of the desired product Int-3A as an orange solid (Yield: 100%).

### Step 4 Preparation of 2-(5-Chloro-4-methyl-2-nitro-phenylamino)-tetrahydro-pyran-3,4,5-triol [Int-3B]

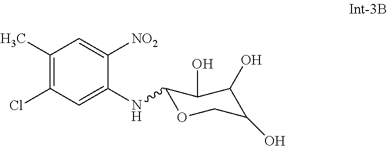

A solution of 5-chloro-4-methyl-2-nitro-phenylamine [Int-3A] (19.8 g, 0.1 mol), ammonium chloride (100 mg), and D-ribose (15.9 g, 0.1 mol) in EtOH (200 mL) is set to reflux temperature under an argon atmosphere and stirred for 12 h. The solution is concentrated under reduced pressure, suspended in DCM:MeOH (1:1), and filtered to remove unreacted starting material. The mother liquor is dry loaded on silica gel (20% MeOH in DCM) and purified by ISCO flash column chromatography. Starting material is eluted using DCM, and the product is eluted using 20% MeOH/DCM. The desired product (7.7 g) Int-3B is isolated as a sticky orange solid (Yield: 24%). Unreacted starting material (12.6 g) is also recovered.

### Step 5 Preparation of (2R,3S,4S)-5-(2-amino-5-chloro-4-methylphenylamino)-pentane-1,2,3,4-tetraol

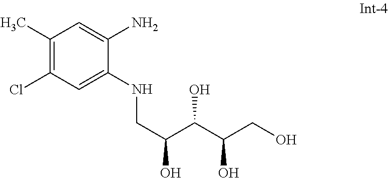

To a solution of 2-(5-chloro-4-methyl-2-nitro-phenylamino)-tetrahydro-pyran-3,4,5-triol [Int-3B] (20 g, 0.63 mol) in EtOH (200 mL), is added sodium borohydride, portionwise, such that evolution of gas is not too vigorous. The resulting mixture is heated at reflux for 12 h. The reaction mixture is then cooled to 0° C. and palladium/carbon (400 mg) is added and the mixture is stirred at room temperature for 1 h. An additional portion of sodium borohydride (3.0 g) is added to complete the reduction, which is indicated by the reaction becoming colourless. The reaction mixture is filtered through Celite, which is washed liberally with MeOH, and the solution is then concentrated to obtain the crude product Int-4 as a clear, colourless oil to be used directly in the next step. LC-MS m/z 290.9 [M+H], retention time 1.38 min.

### Step 6 Preparation of 8-chloro-7-methyl-104(2S,3S,4R)-2,3,4,5-tetrahydroxypentyl)-benzo[g]pteridine-2,4(3H,10H)-dione

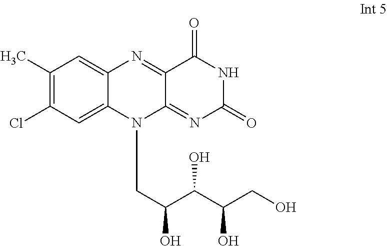

Crude 5-(2-amino-5-chloro-4-methyl-phenylamino)-pentane-1,2,3,4-tetraol (0.56 mmol) is dissolved in glacial acetic acid (6 mL) and stirred at room temperature. The flask is purged with argon for 20 min, following which alloxan monohydrate (90 mg, 0.56 mmol) and boron oxide (78 mg, 1.12 mmol) are added to the stirring solution. The reaction is maintained under an argon atmosphere and heated to 70° C. with stirring. After 14 h, the reaction is cooled to room temperature. Yellow precipitate is observed in solution. The solution is concentrated under reduced pressure, and the residue dissolved in DMSO (2 mL), filtered, and purified by preparatory HPLC (Method 1). 8-Chloro-7-methyl-10-(2,3,4,5-tetrahydroxy-pentyl)-10H-benzo[g]pteridine-2,4-dione (20 mg) is isolated following lyophilization of the appropriate fractions (Yield: 9%). LC-MS m/z 397.1 [M+H], retention time 1.58 min. ¹H NMR (400 MHz, DMSO-d6) δ 2.51 (s, 3H), 3.46 (m, 1H), 3.64 (m, 2H), 4.23 (m, 1H), 4.49 (m, 1H), 4.67 (m, 1H), 4.78 (m, 2H), 4.88 (m, 1H), 5.15 (m, 2H), 8.13 (s, 1H), 8.20 (s, 1H), 11.47 (s, 1H).

### Step 7 Preparation of 8-(2-methoxyethylamino)-7-methyl-10-(2S,3S,4R)-2,3,4,5-tetrahydroxypentyl)benzo[g]pteridine-2,4(3H,10H)-dione

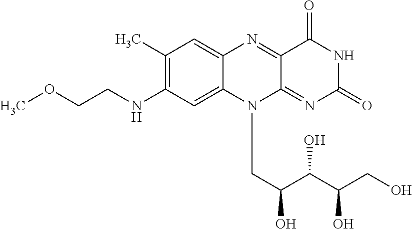

To a solution of 8-chloro-7-methyl-10-(2,3,4,5-tetrahydroxy-pentyl)-10H-benzo[g]pteridine-2,4-dione (50 mg, 0.13 mmol) in DMSO (2 mL) at room temperature, is added 2-methoxyethylamine (47 mg, 0.63 mmol), and the solution is stirred at 70° C. for 12 h. The reaction is cooled to room temperature, diluted with 3 mL of water, and purified by preparatory HPLC (Method 1). The desired product (45 mg) is isolated as a fluffy red solid (Yield: 82%). ¹H NMR (400 MHz, DMSO-d₆) δ 10.99 (S, 1H), 7.67 (S, 1H), 7.10 (br, 1H), 6.87 (S, 1H), 5.00 (br, 2H), 4.50 (br, 2H), 4.24 (br, 2H), 3.3-3.6 (m, 11H), 2.27 (s, 3H); LC-MS m/z 436.3 (M+H), retention time 4.32 min.

### Example 5

### 8-amino-7-methyl-10-((2S,3S,4R)-2,3,4,5-tetrahydroxypentyl)benzo[g]-pteridine-2,4(3H,10H)-dione

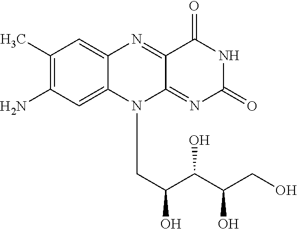

### Step 1 Preparation of 2-(4-Methyl-3-nitro-phenylamino)-tetrahydro-pyran-3,4,5-triol

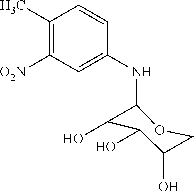

A solution of 4-methyl-3-nitro-phenylamine (2.0 g, 13.14 mmol), ammonium chloride (30 mg), and D-ribose (1.973 g, 13.14 mmol) in EtOH (75 mL) is refluxed under an argon atmosphere for 12 h. The solution is concentrated under reduced pressure. The residue is dry loaded on silica gel (20% MeOH in DCM) and purified by ISCO flash column chromatography using 20% MeOH in dichloromethane as eluent. Desired product (1.8 g) is isolated (Yield: 48%).

### Step 2 Preparation of 5-(4-Methyl-3-nitro-phenylamino)-pentane-1,2,3,4-tetraol

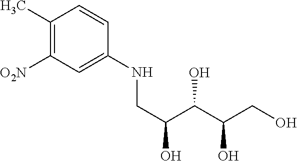

To an ice cooled solution of 2-(4-methyl-3-nitro-phenylamino)-tetrahydro-pyran-3,4,5-triol (1.8 g, 6.3 mmol) in EtOH (15 mL), is added sodium borohydride (1.7 g, 44 mmol). The reaction mixture is heated to reflux for 2.5 h. The reaction is cooled to room temperature and excess sodium borohydride is quenched using 1 M aqueous HCl. The mixture is neutralized using saturated, aqueous NaHCO₃ solution, and concentrated under reduced pressure. The residue is purified by flash column chromatography using methanol/tetrahydrofuran (0%-20%) as eluent to afford 1.2 g of the desired product. (Yield: 66%) LC-MS m/z 287.1 (M+H), retention time 1.95 min.

### Step 3 Preparation of 5-(3-Amino-4-methyl-phenylamino)-pentane-1,2,3,4-tetraol

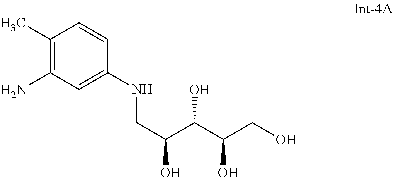

To a suspension of 5-(4-methyl-3-nitro-phenylamino)-pentane-1,2,3,4-tetraol (1.2 g, 4.2 mmol) in 25 mL of 30% aqueous ammonia and 10 ml ethanol cooled to 0° C., is added zinc dust (1.37 g, 21.0 mmol). The reaction mixture is stirred at 0° C. for 30 min., and then at room temperature for 5 h. After the reaction is complete (monitored by TLC) the mixture is filtered through a Celite pad, and the pad is rinsed with dichloromethane. The filtrate is concentrated under reduced pressure and the resulting material used in the next step without further purification. LC-MS m/z 257.1 (M+H), retention time 0.74 min.

### Step 4 Preparation of 8-Amino-7-methyl-10-(2,3,4,5-tetrahydroxy-pentyl)-10H-benzo[g]pteridine-2,4-dione

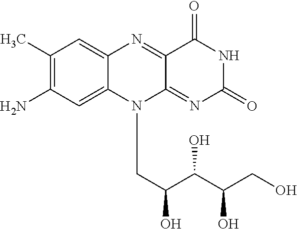

To a solution of 5-(3-amino-4-methyl-phenylamino)-pentane-1,2,3,4-tetraol [Int-4A] (crude product from step 3) in 20 mL MeOH, is added violuric acid monohydrate (740 mg, 4.20 mmol). The reaction mixture is heated to 70° C. for 18 hours. The solid is removed by filtration and the filtrate is concentrated and the resulting material is purified by preparatory HPLC (Method 1) to obtain 4.0 mg of the desired product (2% yield). ¹H NMR (400 MHz, DMSO-d₆) δ 2.24 (s, 3H), 3.56 (dd, 1H), 3.67 (m, 2H), 4.26 (d, 1H), 4.49 (d, 2H), 4.76 (t, 2H), 6.96 (s, 1H), 7.18 (br, 1H), 7.66 (s, 1H), 10.97 (s, 1H); LC-MS m/z 378.1 (M+H), retention time 1.00 min.

### Example 6

### 12-(2S,3S,4R,5-Tetrahydroxy-pentyl)-8,9-dihydro-12H-7,10-dioxa-1,3,5,12-tetraaza-napthacene-2,4-dione

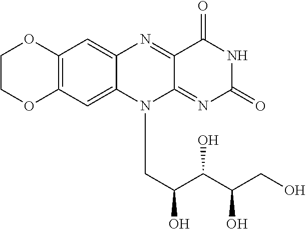

### Step 1 Preparation of 5-(2,3-Dihydro-benzo[1,4]dioxin-6-ylamino)-pentane-1,2R,3S,4S-tetraol

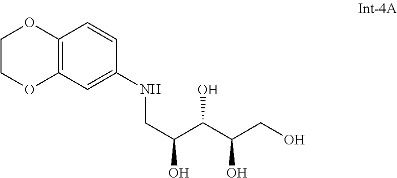

To a solution of 1,4-benzodioxan-6-amine (1.152 g, 7.620 mmol) in MeOH (15 mL) is added D-ribose (2.287 g, 15.24 mmol) and sodium cyanoborohydride (0.957 g, 15.24 mmol) and the mixture is heated to 70° C. for 2 h. The reaction is cooled to room temperature and 1 M aqueous HCl is added causing evolution of gas. The mixture is neutralized with saturated NaHCO₃ solution (10 mL). The product is extracted using EtOAc (5×30 mL). The organic layer is dried over Na₂SO₄, filtered, and concentrated to dryness. The crude product is used in the following step without further purification. LC-MS m/z 286.3 (M+H), retention time 0.71 min.

### Step 2 Preparation of 12-(2,3,4,5-Tetrahydroxy-pentyl)-8,9-dihydro-12H-7,10-dioxa-1,3,5,12-tetraaza-napthacene-2,4-dione

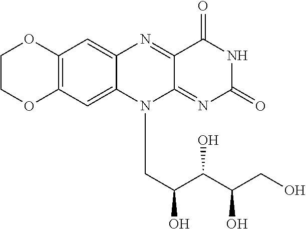

The crude compound obtained from the previous step, Int-4A, is dissolved in EtOH/H₂O (8 mL/8 mL) and violuric acid monohydrate (945 mg, 5.4 mmol) is added to the mixture, which is then heated to 110° C. for 18 hours. A precipitate forms and is filtered. The filtrate is concentrated, dissolved in 9 mL water and 1 mL DMSO, and purified by preparative HPLC (Method 1). After purification, 66.2 mg of desired product is isolated (2% yield, 2 steps). ¹H NMR (400 MHz, DMSO-d₆) δ 3.47 (br, 4H), 3.66 (m, 2H), 4.26 (m, 1H), 4.44 (m, 2H), 4.53 (m, 2H), 4.65 (d, 2H), 4.83 (t, 2H), 7.60 (s, 1H), 7.63 (s, 1H), 11.33 (s, 1H); LC-MS m/z 286.1 (M+H), retention time 1.15 min.

### Example 7

### 7-(8-Dimethylamino-2,4-dioxo-3,4-dihydro-2H-benzo[g]pteridin-10-14)-heptanoic acid

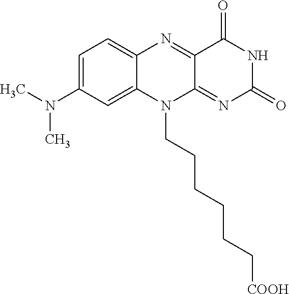

Step 1 Preparation of 7-(3-Dimethylamino-phenylamino)-heptanoic acid [Int-4A]

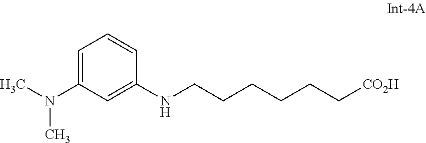

To a mixture of N,N-dimethyl-m-phenylenediamine dihydrochloride (663 mg, 3.17 mmol) and 7-bromoheptanoic acid (662 mg, 3.17 mmol) in 10 mL EtOH is added triethylamine (2.2 mL, 16 mmol). The reaction mixture becomes homogenous and is stirred at 90° C. for 21 h. The reaction is cooled to room temperature and solvent is removed under reduced pressure. The crude product Int-4A is used directly in the following step. LC-MS m/z 265.2 (M+H), retention time 2.33 min.

### Step 2 Preparation of 7-(8-Dimethylamino-2,4-dioxo-3,4-dihydro-2H-benzo[i]pteridin-10yl)-heptanoic acid

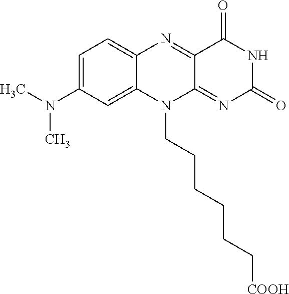

Violuric acid monohydrate (164 mg, 0.94 mmol) and crude 7-(3-dimethylamino-phenylamino)-heptanoic acid (250 mg, 0.940 mol) are dissolved in MeOH/H₂O (5 mL/5 mL) and the solution is heated to 70° C. for 18 h. The solvent is concentrated to dryness. The crude residue is dissolved in 6 mL of DMSO and purified by preparatory HPLC (Method 1). The product precipitates as a red solid in collected fractions. The solid is filtered, and washed with water and diethyl ether. Residual solvent is removed under vacuum and 6.91 mg of desired product is obtained (2% yield). ¹H NMR (400 MHz, DMSO-d₆) δ 1.58-1.35 (m, 6H), 1.73 (br, 2H), 2.22 (m, 2H), 3.25 (s, 3H), 3.27 (s, 3H), 4.56 (br, 2H), 6.54 (s, 1H), 7.25 (br, 1H), 7.84 (m, 1H), 10.98 (s, 1H), 11.98 (s, 1H); LC-MS m/z 385.9 (M+H), retention time 2.54 min.

### Example 8

### 6-(8-Dimethylamino-7-methyl-2,4-dioxo-3,4-dihydro-2H-benzo[g]pteridin-10-yl)-hexanoic acid

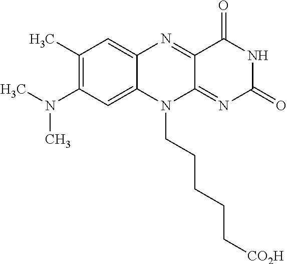

### Step 1 Preparation of 6-(3-Dimethylamino-4-methyl-phenylamino)-hexanoic acid

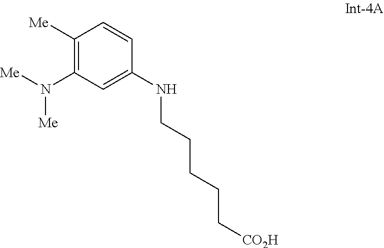

N¹,N¹,6-trimethylbenzene-1,3-diamine (794 mg, 5.28 mmol) and 6-bromo-hexanoic acid (1.030 g, 5.28 mmol) are dissolved in 10 mL EtOH, following which triethylamine (3.7 mL, 26.4 mmol) is added to this solution. The reaction mixture is then stirred at 95-100° C. After 4 h, 225 mg of 6-bromo-hexanoic acid is added to complete the reaction. Heating is continued for 3 more hours, following which the solution is cooled and kept at room temperature overnight. LC-MS analysis indicates the mixture contains approximately 20% dialkylated product. Solvent is removed and the crude residue Int-4A is dissolved in 15 mL MeOH and 15 mL water. Half of this mixture is used in the following step. LC-MS m/z 265.1 (M+H), retention time 1.59 min.

### Step 2 Preparation of 6-(8-Dimethylamino-7-methyl-2,4-dioxo-3,4-dihydro-2H-benzo[g]pteridin-10-yl)-hexanoic acid

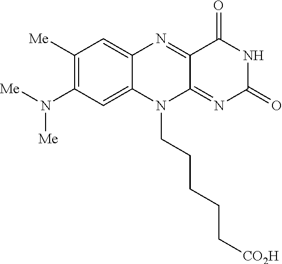

Into a 15 mL (1:1 MeOH:H₂O) solution containing the crude product from the previous step is added violuric acid monohydrate (462 mg, 4.64 mmol) and the mixture is heated to 95° C. for 6 hours. The reaction is cooled to room temperature and kept at that temperature for 2 days. A red solid precipitates in the reaction mixture, which is filtered, and the precipitate is washed with water (10 mL) and ether (10 mL). Recrystallization of the precipitate is performed by dissolving the solid in 5 mL of hot MeOH and crashing out the product by adding 2 mL of water. The solid is filtered, washed with 30 mL water and then 20 mL Et₂O. The red solid is dried under vacuum and 178.4 mg of desired product is isolated. The filtrate is concentrated, dissolved in 9 mL of DMSO, and purified by preparative HPLC to produce 19.2 mg of the desired product. A total of 197.6 mg of the desired product is isolated (24% yield). ¹H NMR (400 MHz, DMSO-d₆) δ 1.48 (m, 2H), 1.62 (m, 2H), 1.75 (m, 2H), 2.25 (t, 2H), 2.46 (s, 3H), 3.06 (s, 6H), 4.59 (br, 2H), 6.86 (S, 1H), 7.80 (s, 1H), 11.10 (s, 1H), 12.01 (s, 1H); LC-MS m/z 385.9 (M+H), retention time 2.64 min.

### Example 9

### 7-(8-Dimethylamino-7-methyl-2,4-dioxo-3,4-dihydro-2H-benzo[g]pteridin-10-yl)-heptanoic acid methyl ester

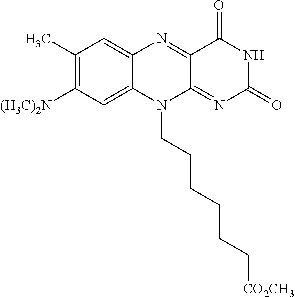

### Step 1 Preparation of 7-(8-Dimethylamino-7-methyl-2,4-dioxo-3,4-dihydro-2H-benzo[g]pteridin-10-yl)-heptanoic acid methyl ester

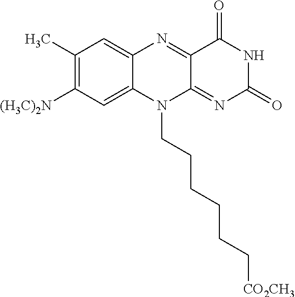

To a solution of 7-(8-dimethylamino-7-methyl-2,4-dioxo-3,4-dihydro-2H-benzo[g]pteridin-10-yl)-heptanoic acid (20 mg, 0.050 mmol) in anhydrous methanol (3 mL) is added concentrated sulfuric acid (30 μL). The reaction mixture is heated to reflux and stirred for 14 h and then cooled to room temperature and neutralized with triethylamine. The solution is filtered and purified by preparative HPLC (Method 1). 7-(8-Dimethylamino-7-methyl-2,4-dioxo-3,4-dihydro-2H-benzo[g]pteridin-10-yl)-heptanoic acid methyl ester (12 mg) is isolated following lyophilization (Yield: 58.0%). ¹H NMR (400 MHz, DMSO-d₆) δ 1.42 (m, 4H), 1.56 (m, 2H), 1.73 (m, 2H), 2.30 (m, 2H), 2.45 (s, 3H), 3.06 (s, 6H), 3.58 (s, 3H), 4.58 (m, 2H), 6.86 (s, 1H), 7.80 (s, 1H), 11.11 (s, 1H); LC-MS m/z 414.2 [M+H], retention time 3.63 min.

### Example 10

### 10-Butyl-8-dimethylamino-7-methyl-10H-benzo[g]pteridine-2,4-dione

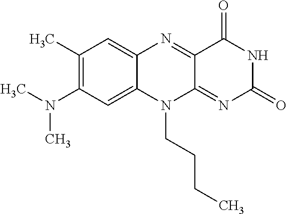

### Step 1 Preparation of 1,5-Dichloro-2-methyl-4-nitro-benzene

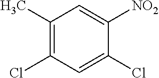

To a solution of 2,4-dichloro-1-methyl-benzene (10.3 g, 0.064 mol) in 60 mL of concentrated sulfuric acid at −10° C., is added nitric acid (2 mL, 0.06 mol) dropwise over 0.5 h, maintaining the reaction temperature at or below −10° C. After the reaction is complete, as monitored by TLC, the reaction mixture is poured into 200 mL of ice and the solid is filtered and washed with water. The crude is recrystallized from hexane and filtered to yield 10.4 g of the desired product as a yellow solid (Yield: 80%).

### Step 2 Preparation of Butyl-(5-chloro-4-methyl-2-nitro-phenyl)-amine [Int-3]

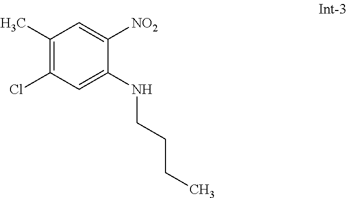

To a solution of 1,5-dichloro-2-methyl-4-nitro-benzene (900 mg, 4.37 mmol) in 50 mL of THF is added n-butylamine (1.6 g, 2.18 mL, 21.8 mmol) and triethylamine (660 mg, 0.9 mL, 6.56 mmol). The reaction mixture is heated to reflux for 15 h. The reaction mixture is concentrated under reduced pressure and the crude product purified by flash column chromatography using dichlormethane/hexanes (10%-30%) as eluent to yield 844 mg of the desired Int-3 product (Yield: 80%). LC-MS m/z 243.1 (M+H), retention time 6.90 min.

### Step 3 Preparation of N²-Butyl-4-chloro-5-methyl-benzene-1,2-diamine

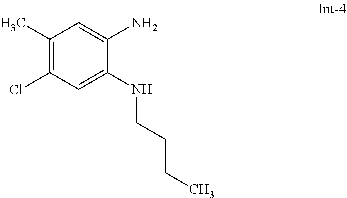

To a suspension of butyl-(5-chloro-4-methyl-2-nitro-phenyl)-amine [Int-3] (1.1 g, 4.53 mmol) in 10 mL of 30% aqueous ammonia and 10 mL of ethanol at 0° C., is added zinc dust (1.5 g, 22.8 mmol). The reaction is stirred at 0° C. for 15 min, at which point the ice bath is removed, and the reaction is stirred at room temperature for 5 h. After the reaction is complete, as monitored by TLC, the reaction mixture is filtered through a Celite pad, and rinsed with dichloromethane. The filtrate is concentrated under reduced pressure and purified by flash column chromatography using dichloromethane/hexanes (10%-100%) as eluent to yield 624 mg of the desired Int-4 product (Yield: 71%). LC-MS m/z 213.1 (M+H), retention time 3.34 min.

### Step 4 Preparation of 10-Butyl-8-chloro-7-methyl-10H-benzo[g]pteridine-2,4-dione

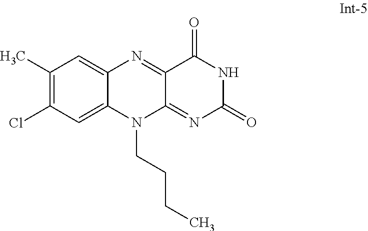

To a solution of N²-butyl-4-chloro-5-methyl-benzene-1,2-diamine [Int-4] (624 mg, 3.14 mmol) in 15 mL of acetic acid, under an argon atmosphere, is added alloxan monohydrate (503 mg, 3.14 mmol) and boron oxide (437 mg, 6.28 mmol). The reaction mixture is stirred at 60° C. for 3 h, cooled to room temperature, and stirred for 16 h. The reaction mixture is diluted with 50 mL water, neutralized with aqueous sodium bicarbonate, and extracted with dichloromethane (3×50 mL). The organic layers are combined, dried over magnesium sulfate, and concentrated under reduced pressure. The crude Int-5 product is purified by flash column chromatography using methanol/dichloromethane (0%-10%) as eluent to yield 600 mg of the desired Int-5 product (Yield: 60%). LC-MS m/z 319.1 (M+H), retention time 3.59 min.

### Step 5 Preparation of 10-Butyl-8-dimethylamino-7-methyl-10H-benzo[g]pteridine-2,4-dione

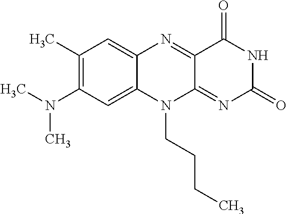

To a solution of 10-butyl-8-chloro-7-methyl-10H-benzo[g]pteridine-2,4-dione [Int-5] (80 mg, 0.25 mmol) in 3 mL of DMF is added N,N-dimethylamine (0.32 mL, 2M solution in THF, 0.63 mmol). The reaction mixture is heated at 100° C. for 12 h, cooled to room temperature, and purified by preparative HPLC (Method 1) to yield 12 mg of the desired product (Yield: 15%). ¹H NMR (400 MHz, DMSO-d₆) δ 0.99 (t, 3H), 1.46 (m, 2H), 1.73 (m, 2H), 2.45 (s, 3H), 3.06 (s, 6H), 4.5-4.7 (br, 2H), 6.86 (s, 1H), 7.80 (s, 1H), 11.13 (s, 1H); LC-MS m/z 328.3 (M+H), retention time 3.23 min.

### Example 11

### 5-(8-Dimethylamino-7-methyl-2,4-dioxo-3A-dihydro-2H-benzo[g]pteridin-10-yl)-pentanoic acid

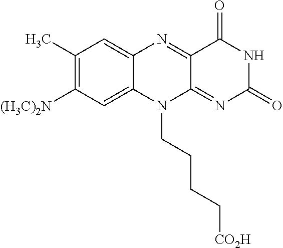

### Step 1 Preparation of 5-(3-Dimethylamino-4-methyl-phenylamino)-pentanenitrile

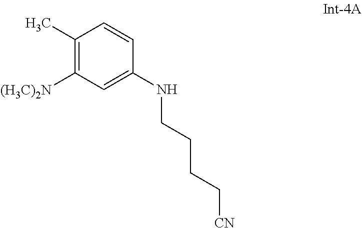

To a solution of 3-dimethylamino-4-methylaniline (632 mg, 4.21 mmol) in anhydrous ethanol (10 mL) is added triethylamine (1.76 mL, 12.63 mmol) and 5-bromovaleronitrile (682 mg, 4.21 mmol). The stirring solution is heated at reflux for 14 h. The solution is concentrated, and the crude product Int-4A is used directly in the following step. LC-MS m/z 232.1 [M+H], retention time 1.52 min.

### Step 2 Preparation of 5-(8-Dimethylamino-7-methyl-2,4-dioxo-3,4-dihydro-2H-benzo[g]pteridin-10-yl)-pentanenitrile

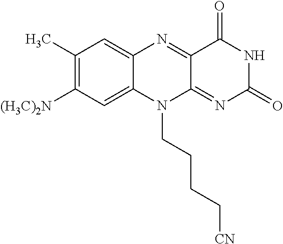

Crude 5-(3-dimethylamino-4-methyl-phenylamino)-pentanenitrile [Int-4A] (2 mmol) is dissolved in methanol (8 mL). Violuric acid monohydrate (350 mg, 2 mmol) is dissolved in water (8 mL) in a separate flask, and heated at 60° C. with stirring. The solution of 5-(3-dimethylamino-4-methyl-phenylamino)-pentanenitrile in methanol is added to the aqueous solution of violuric acid, and the mixture heated at 60° C. with stirring. After 6 h, the reaction mixture is removed from the heat, and the mixture is stored at room temperature in the dark for 16 h. The reaction mixture is concentrated, and the precipitate is filtered. The mother liquor is diluted with DMSO (5 mL) and purified by preparative HPLC (Method 1). The precipitate (38 mg) is also dissolved in DMSO and purified by preparative HPLC (Method 1). Lyophilization of combined fractions from the two preparative HPLC runs affords 713 mg (2.02 mmol) of desired product (Yield: 48.0% for two steps). ¹H NMR (400 MHz, DMSO-d₆) δ 1.75 (m, 2H), 1.88 (m, 2H), 2.46 (s, 3H), 2.64 (m, 2H), 3.07 (s, 6H), 4.65 (m, 2H), 6.89 (s, 1H), 7.81 (s, 1H), 11.11 (s, 1H); ESI(+) m/z 353.3 [M+H].

### Step 3 Preparation of 5-(8-Dimethylamino-7-methyl-2,4-dioxo-3,4-dihydro-2H-benzo[g]pteridin-10-yl)-pentanoic acid

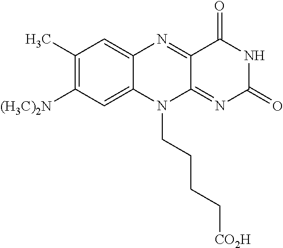

5-(8-Dimethylamino-7-methyl-2,4-dioxo-3,4-dihydro-2H-benzo[g]pteridin-10-yl)-pentanenitrile (514 mg, 1.46 mmol) is dissolved in 6 N aqueous HCl (8 mL), and the solution is heated at reflux with stirring. After 5 h, TLC indicates consumption of the starting material. The reaction mixture is cooled to room temperature and neutralized with triethylamine. The mixture is then filtered and purified by preparative HPLC (Method 1). HPLC fractions are allowed to stand in the dark at room temperature for 24 h. Fractions containing product (as indicated by TLC 10% MeOH in dichloromethane) are filtered, and the precipitate washed with water and air-dried. 5-(8-Dimethylamino-7-methyl-2,4-dioxo-3,4-dihydro-2H-benzo[g]pteridin-10-yl)-pentanoic acid (10 mg) is isolated (Yield: 1.8%). ¹H NMR (400 MHz, DMSO-d₆) δ 1.67 (m, 2H), 1.76 (m, 2H), 2.34 (t, 2H), 2.45 (s, 3H), 3.06 (s, 6H), 4.59 (m, 2H), 6.86 (s, 1H), 7.79 (s, 1H), 11.12 (s, 1H), 12.11 (br s, 1H); LC-MS m/z 372.2 [M+H], retention time 2.32 min.

### Example 12

### Preparation of 3-[7-Methyl-2,4-dioxo-10-(2,3,4,5-tetrahydroxy-pentyl)-2,3,4,10-tetrahydro-benzo pteridin-8-ylamino]-propionic acid

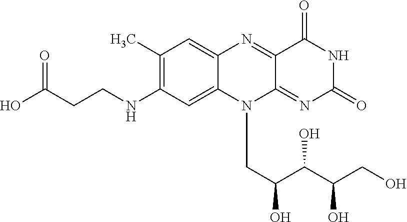

To a solution of 3-[7-methyl-2,4-dioxo-10-(2,3,4,5-tetrahydroxy-pentyl)-2,3,4,10-tetrahydro-benzo[g]pteridin-8-ylamino]-propionic acid tert-butyl ester (15 mg, 0.03 mmol) (prepared using the procedure for example 4, step 7) in DCM (2 mL) at room temperature is added TFA (2 mL), and the solution is stirred for 2 h. The solution is concentrated under reduced pressure, dissolved in ACN/water, and lyophilized to yield 5.3 mg of the product as a fluffy orange solid (Yield: 41%). ¹H NMR (400 MHz, DMSO-d₆) δ 12.36 (s, 1H), 10.96 (s, 1H), 7.07 (s, 1H), 6.86 (S, 1H), 5.4-4.2 (m, 7H), 3.63 (m, 2H), 2.68 (m, 2H), 2.26 (s, 3H); LC-MS m/z 450.3 (M+H), retention time 4.24 min.

### Example 13

### [6-(8-Dimethylamino-7-methyl-2,4-dioxo-3,4-dihydro-2H-benzo[g]pteridin-10-yl)-hexyl]-phosphonic acid

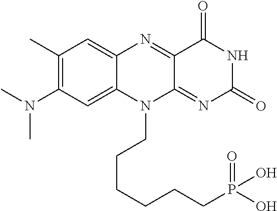

### Step 1 Preparation of Diethyl 6-bromohexylphosphonate

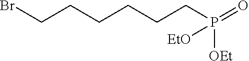

1,6-Dibromohexane (2 g, 8.2 mmol) and P(OEt)₃ (6.81 g, 4.1 mmol) are combined neat in a round bottom flask (25 mL). The mixture is then heated in an oil bath at 150° C. The progress of the reaction is monitored by LC-MS and the reaction is complete after 3 h. A mixture of monophosphonate and bisphosphonate is obtained. The desired compound is found by LC-MS to have m/z 302.9 (M+H), and retention time 4.84 min. The undesired product has LC-MS m/z 359.1 (M+H), and retention time 4.21 min. The compounds are visualized on TLC (100% hexane) by staining with phosphomolybdic acid solution. Crude product is used directly in the following step.

### Step 2 Preparation of Diethyl 6-(3-(dimethylamino)-4 methylphenylamino)-hexylphosphonate

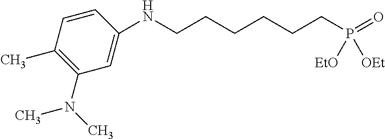

This compound is prepared using the procedure of Example 8, Step 1 and using diethyl 6-bromohexylphosphonate (1.23 g, 4.1 mmol) and N¹,N¹,6-trimethylbenzene-1,3-diamine (616 mg, 4.1 mmol). The monoalkylated product has LC-MS m/z 371.2 (M+H), retention time 3.18 min. The dialkylated product has LC-MS m/z 591.3 (M+H), retention time 4.38 min. The crude product is used in the next step without purification.

### Step 3 Preparation of Diethyl 6-(8-(dimethylamino)-7-methyl-2,4-dioxo-3,4-dihydrobenzo[g]pteridin-10(2H)-yl)hexylphosphonate

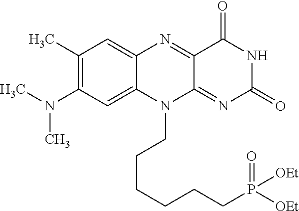

This compound is prepared using the procedure of Example 8, Step 2 and using diethyl 6-(3-(dimethylamino)-4 methylphenylamino)hexylphosphonate from step 2 and violuric acid monohydrate (718 mg, 4.4 mmol). The product is isolated by preparative HPLC (Method 1) as a red solid with 2.5% overall yield (50 mg, 0.1 mmol) for the 3 steps. ¹H NMR (300 MHz, CD₃OD) δ 7.83 (1H, s), 6.89 (1H, s),), 4.75 (2H, m), 4.10 (4H, m), 3.20 (6H, s), 2.54 (3H, s), 1.85 (4H, m), 1.63 (6H, m), 1.33 (6H, t); LC-MS m/z 492.2 (M+H), retention time 3.72 min.

### Step 4 Preparation of 6-(8-(Dimethylamino)-7-methyl-2,4-dioxo-3,4-dihydrobenzo[g]pteridin-10(2H)-yl)hexylphosphonic acid

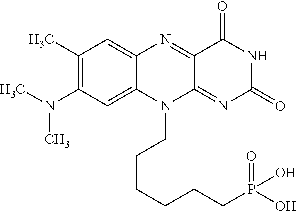

Diethyl 6-(8-(dimethylamino)-7-methyl-2,4-dioxo-3,4-dihydrobenzo[g]-pteridin-10(2H)-yl)hexylphosphonate (10 mg, 0.02 mmol) is dissolved in 6N aqueous HCl (5 mL) in a round bottom flask (10 mL) and heated at 100° C. in an oil bath for 12 hours. The reaction is monitored by LC-MS. Solvent is removed under reduced pressure and the residual solid is purified by HPLC (Preparative Method 2) and the desired product is isolated in 35% yield as a red solid (3.5 mg), LC-MS m/z 436.2 (M+H), retention time 2.53 min.

### Example 14

### (7,8-Dimethyl-2,4-dioxo-3,4-dihydro-2H-benzo[g]pteridin-10-yl)-acetaldehyde

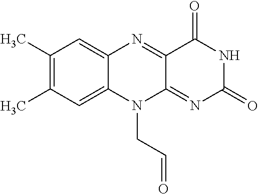

To a suspension of riboflavin (2.85 g, 7.5 mmol) in 2 N aqueous sulfuric acid (75 mL), cooled to 0° C. in a flask covered with tinfoil, is added orthoperiodic acid (6.3 g, 27.5 mmol). After 30 min, the reaction is allowed to warm to room temperature. After 2 h, the pH of the reaction solution is adjusted carefully to 1.5 by addition of solid sodium carbonate. The precipitate is then filtered off and the filtrate is basified (using solid sodium carbonate) to pH=3.9. The precipitate is filtered, washed liberally with cold water, ethanol, and diethyl ether to yield 2.0 g of the desired product as an orange solid (Yield: 94%). LC-MS m/z 285.1 [M+H].

### Example 15

### N-(3-(2-(7,8-dimethyl-2,4-dioxo-3,4-dihydrobenzo[g]pteridin-10(2H)-yl)ethylamino)propyl)-1,1,1-trifluoromethanesulfonamide

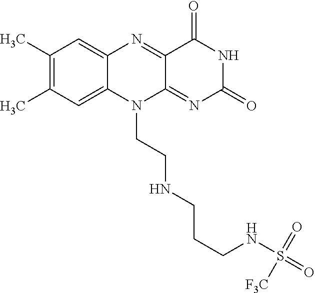

### Step 1 N-(3-aminopropyl)-1,1,1-trifluoromethanesulfonamide

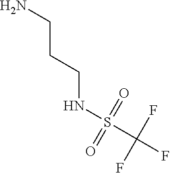

To a solution of 1,3-diaminopropane (2 mL, 24 mmol) and triethylamine (3.3 mL, 24 mmol) in anhydrous CH₂Cl₂ (10 mL) at 0° C., is added trifluoromethanesulfonic anhydride (0.8 mL, 5 mmol) dropwise over 10 min. After completion of the addition, the reaction mixture is removed from the ice bath, and then stirred at room temperature for 7 h. The reaction is diluted with water (20 mL) and the organic layer is separated, and washed with brine (20 mL). The organic layer is dried over Na₂SO₄, filtered, and concentrated under reduced pressure. The crude residue is then triturated with diethyl ether:ethyl acetate (1:1) and the solid is removed by filtration. The filtrate is concentrated and purified via silica gel chromatography (ISCO) (100% CH₂Cl₂ to 10% MeOH/CH₂Cl₂) over 15 minutes to obtain a yield of 163 mg of desired product as a white semi-solid (Yield: 17%). LC-MS m/z 207.0 [M+H].

### Step 2 Preparation of N-{3-[2-(7,8-Dimethyl-2,4-dioxo-3,4-dihydro-2H-benzo[g]pteridin-10-yl)-ethylamino]-propyl}-C,C,C-trilluoromethanesulfonamide

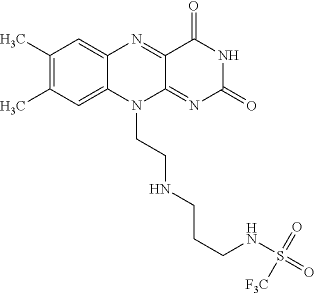

To a suspension of (7,8-dimethyl-2,4-dioxo-3,4-dihydro-2H-benzo[g]pteridin-10-yl)-acetaldehyde (100 mg, 0.35 mmol) in methanol (7 mL) is added N-(3-amino-propyl)-C,C,C-trifluoro-methanesulfonamide (72 mg, 0.35 mmol)) and acetic acid (0.1 mL) at room temperature. After 30 min., sodium cyanoborohydride (48 mg, 0.77 mmol) is added, and the solution is stirred for 16 h. The reaction mixture is concentrated, and the residue is dissolved in DMF/water, filtered, and purified by preparative HPLC (Method 1). N-{3-[2-(7,8-Dimethyl-2,4-dioxo-3,4-dihydro-2H-benzo[g]pteridin-10-yl)-ethylamino]-propyl}-C,C,C-trifluoromethanesulfonamide (21 mg) is isolated following lyophilization of the appropriate fractions (Yield: 13%). ¹H NMR (400 MHz, DMSO-d₆) δ 1.82 (t, 2H), 2.43 (s, 3H), 2.54 (s, 3H), 3.13 (m, 2H), 3.25 (m, 2H), 4.94 (m, 2H), 7.81 (d, 1H), 7.99 (m, 1H), 8.63 (brs, 1H), 9.56 (brs, 1H), 11.49 (s, 1H). LC-MS m/z 475.2 [M−H].

### Example 16

### N-(3-{Bis-[2-(7,8-dimethyl-2,4-dioxo-3,4-dihydro-2H-benzo[g]pteridin-10-yl)-ethyl]-amino}-propyl)-C,C,C-trifluoro-methanesulfonamide

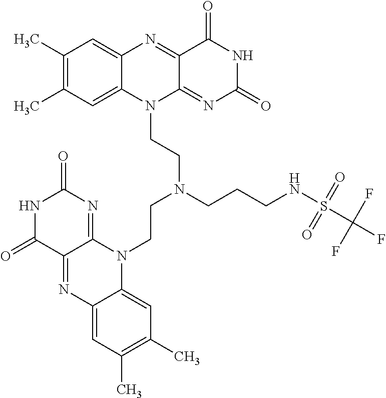

This is a byproduct of the above reaction and isolated from the HPLC run as described in example 15. The product (17 mg) is obtained as an orange solid (Yield: 10%). LC-MS m/z 743.3 [M+H].

### Example 17

### 3-[2-(7,8-Dimethyl-2,4-dioxo-3,4-dihydro-2H-benzo[g]pteridin-10-yl)-ethylamino]-benzoic acid

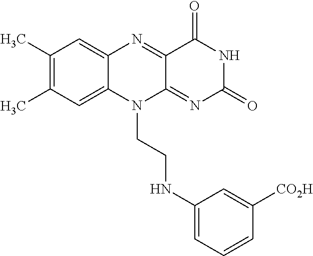

### Step 1 Preparation of 3-[2-(7,8-Dimethyl-2,4-dioxo-3,4-dihydro-2H-benzo[g]pteridin-10-yl)-ethylamino]-benzoic acid t-butyl ester

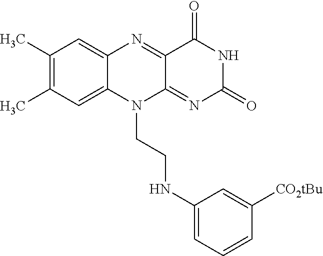

This product is prepared using the procedure of Example 15, Step 2 except (7,8-dimethyl-2,4-dioxo-3,4-dihydro-2H-benzo[g]pteridin-10-yl)-acetaldehyde (100 mg, 0.35 mmol) and tert-butyl 3-aminobenzoate (51 mg, 0.26 mmol) are used to obtain a yield of 22 mg of the desired product as a red powder (Yield: 18.3%). MS m/z 461.9 [M+H].

### Step 2 Preparation of 3-[2-(7,8-Dimethyl-2,4-dioxo-3,4-dihydro-2H-benzo[g]pteridin-10-yl)-ethylamino]-benzoic acid

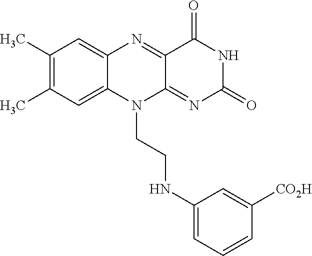

To a suspension of 3-[2-(7,8-dimethyl-2,4-dioxo-3,4-dihydro-2H-benzo[g]pteridin-10-yl)-ethylamino]-benzoic acid t-butyl ester (22 mg, 0.05 mmol) in CH₂Cl₂ (2 mL) is added trifluoroacetic acid (2 mL) at room temperature. After 2 h, the reaction mixture is concentrated and the residual material is dissolved in water/acetonitrile and lyophilized. 3-[2-(7,8-Dimethyl-2,4-dioxo-3,4-dihydro-2H-benzo[g]pteridin-10-yl)-ethylamino]-benzoic acid (17 mg) is isolated (Yield: 83.9%). ¹H NMR (400 MHz, DMSO-d₆) δ 2.29 (s, 3H), 2.36 (s, 3H), 3.60 (m, 2H), 4.74 (m, 2H), 6.90 (d, 1H), 7.07 (s, 1H), 7.16 (m, 2H), 7.53 (s, 1H), 7.86 (s, 1H), 11.36 (s, 1H), LC-MS m/z 404.1 [M−H].

### Example 18

### 7-(8-Dimethylamino-7-methyl-2,4-dioxo-3,4-dihydro-2H-benzo[g]-pteridin-10-yl)-heptanoic acid hydroxyamide

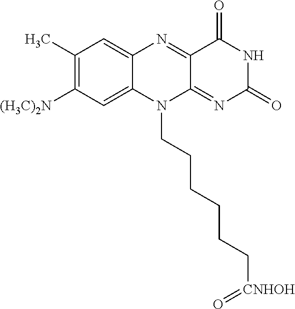

### Step 1 Preparation of 7-(8-Dimethylamino-7-methyl-2,4-dioxo-3,4-dihydro-2H-benzo[g]pteridin-10-yl)-heptanoic acid hydroxyamide

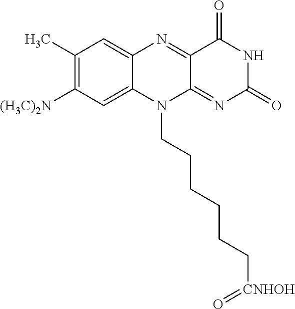

To a solution of 7-(8-dimethylamino-7-methyl-2,4-dioxo-3,4-dihydro-2H-benzo[g]pteridin-10-yl)-heptanoic acid (20 mg, 0.05 mmol) (prepared using a similar procedure to that of Example 8 using the appropriate starting materials) in a mixture of anhydrous THF (15 mL) and anhydrous CH₂Cl₂ (3 mL) at 0° C. (ice/water bath) under an argon atmosphere is added N-methylmorpholine (22 μL, 0.20 mmol) and isobutylchloroformate (26 μL, 0.20 mmol). The ice bath is removed and the solution stirred at room temperature for 2 h. Hydroxylamine hydrochloride (7 mg, 0.10 mmol) is added to the stirring solution at room temperature, and after stirring for 2 h, methanol (2 mL) is added to aid solubility. The solution is stirred for an additional 18 h. The solution is concentrated, and the residue dissolved in a 1:1 mixture of DMSO:water (4 mL). Crude product is purified by preparative HPLC (Method 1). Following lyophilization of appropriate fractions, 7-(8-dimethylamino-7-methyl-2,4-dioxo-3,4-dihydro-2H-benzo[g]pteridin-10-yl)-heptanoic acid hydroxyamide (6.0 mg, 0.0145 mmol) is isolated (Yield: 29%). NMR (400 MHz, DMSO-d₆) δ 1.49 (m, 6H), 1.74 (m, 2H), 1.97 (t, 2H), 2.46 (s, 3H), 3.06 (s, 6H), 4.58 (m, 2H), 6.86 (s, 1H), 7.81 (s, 1H), 8.67 (s, 1H), 10.36 (s, 1H), 11.13 (s, 1H); LC-MS m/z 415.1 [M+H], retention time 4.32 min.

### Example 19

### {2-[2-(7,8-Dimethyl-2,4-dioxo-3,4-dihydro-2H-benzo[g]pteridin-10-yl)-ethylamino]-ethyl}-phosphonic acid

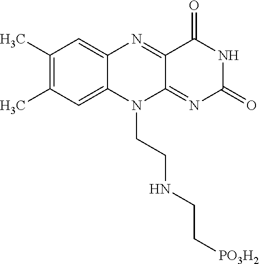

### Step 1 Preparation of {2-[2-(7,8-Dimethyl-2,4-dioxo-3,4-dihydro-2H-benzo[g]pteridin-10-14)-ethylamino]-ethyl}-phosphonic acid

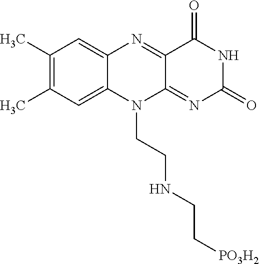

To a suspension of {2-[2-(7,8-dimethyl-2,4-dioxo-3,4-dihydro-2H-benzo[g]pteridin-10-yl)-ethylamino]-ethyl}-phosphonic acid diethyl ester (50 mg, 0.11 mmol) (prepared by reductive amination using a procedure similar to that of Example 15, Step 2 using (7,8-dimethyl-2,4-dioxo-3,4-dihydro-2H-benzo[g]pteridin-10-yl)-acetaldehyde (140 mg, 0.49 mmol) and (2-amino-ethyl)-phosphonic acid diethyl ester (71 mg, 0.39 mmol)) in anhydrous dichloromethane (2 mL) is added bromotrimethylsilane (0.5 mL). The reaction mixture is stirred at room temperature for 16 hours. After completion of the reaction (as monitored by TLC and LC/MS), the reaction is quenched by the addition of water (2 mL) at room temperature. After the solution is stirred for 15 min., methanol is added until the solution becomes homogeneous. The solution is stirred for an additional 15 min, and then concentrated under reduced pressure. The remaining residue is purified by preparative HPLC (Method 1). {2-[2-(7,8-Dimethyl-2,4-dioxo-3,4-dihydro-2H-benzo[g]pteridin-10-yl)-ethylamino]-ethyl}-phosphonic acid (11.0 mg, 0.0280 mmol) is isolated following lyophilization of appropriate fractions (Yield: 25%). ¹H NMR (400 MHz, CD₃OD) δ 2.11 (m, 2H), 2.50 (s, 3H), 2.62 (s, 3H), 3.65 (m, 2H), 5.07 (m, 2H), 7.81 (s, 1H), 8.04 (s, 1H); LC-MS m/z 394.0 [M+H], retention time 1.40 min.

### Example 20

### 4-[2-(7,8-Dimethyl-2,4-dioxo-3,4-dihydro-2H-benzo[g]pteridin-10-yl)-ethylamino]-butyric acid tert-butyl ester

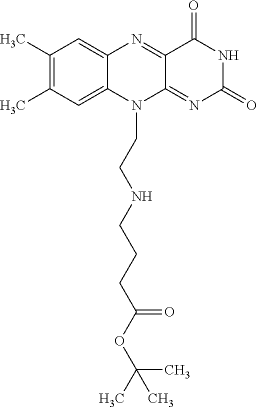

### Step 1 Preparation of 4-[2-(7,8-Dimethyl-2,4-dioxo-3,4-dihydro-2H-benzo[g]pteridin-10-yl)-ethylamino]-butyric acid tert-butyl ester

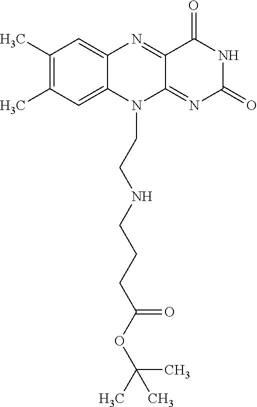

The reductive amination method of Example 15, step 2 is used starting with (7,8-dimethyl-2,4-dioxo-3,4-dihydro-2H-benzo[g]pteridin-10-yl)-acetaldehyde (403 mg, 1.62 mmol) and tert-butyl 4-aminobutanoate (349 mg, 1.78 mmol). The isolated yield of the desired product is 27%. ¹H NMR (400 MHz, d₆-DMSO) δ 1.41 (s, 9H), 1.76 (m, 2H), 2.32 (t, 2H), 2.43 (s, 3H), 2.53 (s, 3H), 3.07 (bm, 2H), 4.93 (t, 2H), 7.81 (s, 1H), 7.98 (s, 1H), 8.54 (bs, 2H), 11.48 (s, 1H), LC-MS m/z 428.2 [M+H] retention time 2.67 min.

### Example 21

### 4-[2-(7,8-Dimethyl-2,4-dioxo-3,4-dihydro-2H-benzo[g]pteridin-10-yl)-ethylamino]-butyric acid

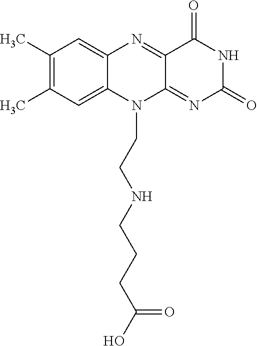

### Step 1 Preparation of 4-[2-(7,8-Dimethyl-2,4-dioxo-3,4-dihydro-2H-benzo[g]pteridin-10-yl)-ethylamino]-butyric acid

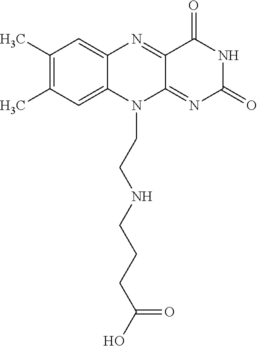

4-[2-(7,8-Dimethyl-2,4-dioxo-3,4-dihydro-2H-benzo[g]pteridin-10-yl)-ethylamino]-butyric acid is prepared by following the procedure of Example 17, Step 2 starting with 4-[2-(7,8-dimethyl-2,4-dioxo-3,4-dihydro-2H-benzo[g]pteridin-10-yl)-ethylamino]-butyric acid tert-butyl ester (120 mg, 0.280 mmol) and trifluoroacetic acid (3 mL) in dichloromethane (5 mL). The isolated yield of the desired product is 86%. ¹H NMR (400 MHz, CD₃OD) δ 2.00 (m, 2H), 2.50 (m, 2H), 2.51 (s, 3H), 2.64 (s, 3H), 3.22 (t, 2H), 3.64 (t, 2H), 5.09 (t, 2H), 7.81 (s, 1H), 8.03 (s, 1H). LC-MS m/z 372.2 [M+H] retention time 1.40 min.

### Example 22

### N-Benzyloxy-4-[2-(7,8-dimethyl-2,4-dioxo-3,4-dihydro-2H-benzo[g]-pteridin-10-yl)-ethylamino]-butyramide

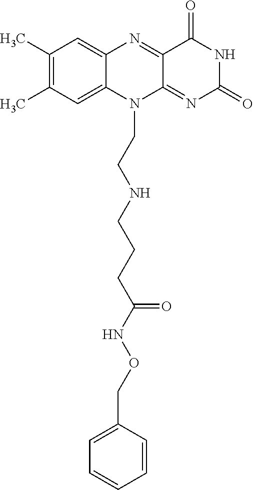

### Step 1 Preparation of 4-{tert-Butoxycarbonyl-[2-(7,8-dimethyl-2,4-dioxo-3,4-dihydro-2H-benzo[g]pteridin-10-yl)-ethyl]-amino}-butyric acid

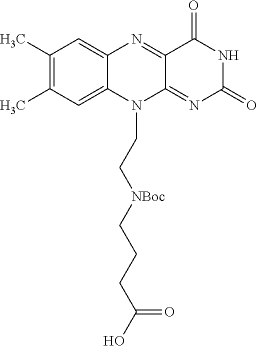

4-[2-(7,8-Dimethyl-2,4-dioxo-3,4-dihydro-2H-benzo[g]pteridin-10-yl)-ethylamino]-butyric acid (Example 21) (234 mg, 0.63 mmol) is dissolved in MeOH (6 mL), triethylamine (0.5 mL) and di-tert-butyl-dicarbonate (206 mg, 0.945 mmol) is added. The reaction mixture is stirred at room temperature for 1 h. Upon completion of the reaction (as monitored by TLC), solvent is removed and the crude product is used in the following step without any purification LC-MS m/z 472.0 [M+H], retention time 3.31 min.

### Step 2 Preparation of (3-Benzyloxycarbamoyl-propyl)-[2-(7,8-dimethyl-2,4-dioxo-3,4-dihydro-2H-benzo[g]pteridin-10-yl)-ethyl]-carbamic acid tert-butyl ester

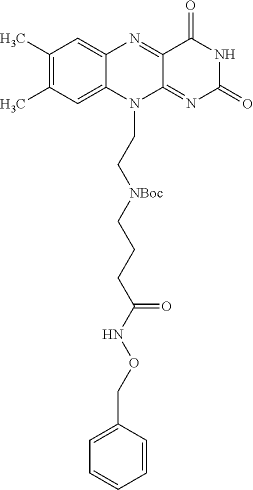

4-{tert-Butoxycarbonyl-[2-(7,8-dimethyl-2,4-dioxo-3,4-dihydro-2H-benzo[g]pteridin-10-yl)-ethyl]-amino}-butyric acid is dissolved in DMF (3 mL). O-benzotriazol-1-yl)-N,N,N′,N′-tetramethyluroniumhexafluoro phosphate (358 mg, 0.945 mmol) is added to this solution and stirred at room temperature for 5 minutes. O-benzylhydroxylamine hydrochloride (201 mg, 1.26 mmol) is added along with diisopropylethylamine (0.5 mL) and the mixture is stirred for 3.5 h. The reaction mixture is diluted with water (6 mL) and purified by preparative HPLC (Method 1). After lyophilization, 161 mg of the desired product is isolated (44% yield). LC-MS m/z 577.0 [M+H], retention time 4.14 min.

### Step 3 Preparation of N-Benzyloxy-4-[2-(7,8-dimethyl-2,4-dioxo-3,4-dihydro-2H-benzo[g]pteridin-10-yl)-ethylamino]-butyramide

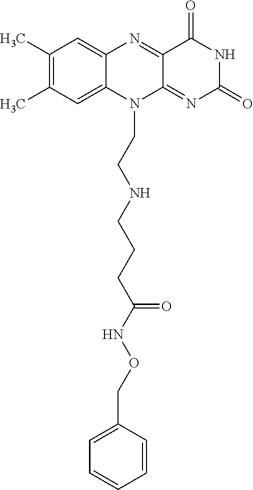

To a solution of (3-benzyloxycarbamoyl-propyl)-[2-(7,8-dimethyl-2,4-dioxo-3,4-dihydro-2H-benzo[g]pteridin-10-yl)-ethyl]-carbamic acid tert-butyl ester (12.9 mg, 0.022 mmol) in dichloromethane (0.5 mL) is added trifluoroacetic acid (0.3 mL). After 3 h of stirring at room temperature, all of the starting material is consumed (as indicated by TLC). The reaction mixture is concentrated under reduced pressure and the residue is dried using a vacuum pump, affording 6.6 mg of the desired product as the TFA salt (63% yield). ¹H NMR (400 MHz, CD₃OD) δ 1.96 (m, 2H), 2.25 (t, 2H), 2.49 (s, 3H), 2.63 (s, 3H), 3.19 (t, 2H), 3.65 (t, 2H), 4.78 (s, 2H), 5.11 (t, 2H), 7.37 (s, 5H), 7.82 (s, 1H), 7.98 (s, 1H), LC-MS m/z 477.0 [M+H], retention time 2.20 min.

### Example 23

### 4-[2-(7,8-Dimethyl-2,4-dioxo-3,4-dihydro-2H-benzo[g]pteridin-10-yl)-ethylamino]-N-hydroxy-butyramide

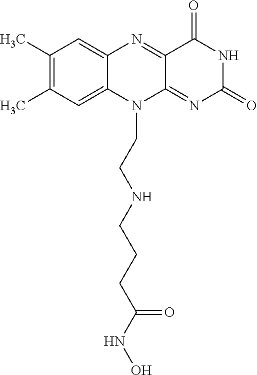

### Step 1 Preparation of [2-(7,8-Dimethyl-2,4-dioxo-3,4-dihydro-2H-benzo[g]pteridin-10-yl)-ethyl]-(3-hydroxycarbamoyl-propyl)-carbamic acid tert-butyl ester

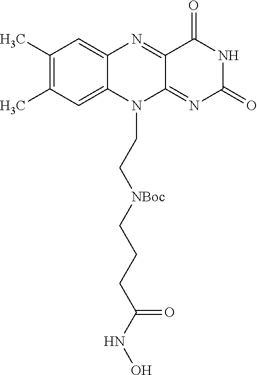

(3-Benzyloxycarbamoyl-propyl)-[2-(7,8-dimethyl-2,4-dioxo-3,4-dihydro-2H-benzo[g]pteridin-10-yl)-ethyl]-carbamic acid tert-butyl ester (150 mg, 0.26 mmol) is dissolved in MeOH (10 mL). The reaction mixture is purged with argon, following which palladium/carbon (10 weight percent, 30 mg) is added. The reaction mixture is placed under an atmosphere of hydrogen at room temperature using a balloon filled with hydrogen. After completion of the reaction (as monitored by TLC) the mixture is filtered through a Celite pad and the Celite is washed with methanol (30 mL). The filtrate is concentrated under reduced pressure and the crude product is used in the final step. LC-MS m/z 486.9 [M+H], retention time 2.48 min.

### Step 2 Preparation of 4-[2-(7,8-Dimethyl-2,4-dioxo-3,4-dihydro-2H-benzo[g]pteridin-10-yl)-ethylamino]-N-hydroxy-butyramide

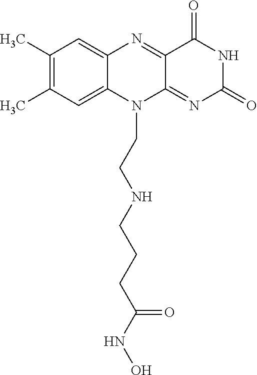

[2-(7,8-Dimethyl-2,4-dioxo-3,4-dihydro-2H-benzo[g]pteridin-10-yl)-ethyl]-(3-hydroxycarbamoyl-propyl)-carbamic acid tert-butyl ester is dissolved in dichloromethane (3.5 mL) and trifluoroacetic acid (1 mL) is added. The mixture is stirred at room temperature for 2 h. The reaction mixture is concentrated under reduced pressure and the crude product is dissolved in water (6 mL) and purified by preparative HPLC (Method 1). The yield of this reaction is 68 mg (68%) after two steps. ¹H NMR (400 MHz, CD₃OD) δ 1.96 (m, 2H), 2.31 (t, 2H), 2.51 (s, 3H), 2.64 (s, 3H), 3.23 (t, 2H), 3.64 (t, 2H), 5.11 (t, 2H), 7.82 (s, 1H), 8.03 (s, 1H), LC-MS m/z 387.1 [M+H], retention time 1.38 min.

### Example 24

### Preparation of 7-(7,8-Dimethyl-2,4-dioxo-3,4-dihydro-2H-benzo[g]pteridin-10-yl)-heptanoic acid tert-butyl ester

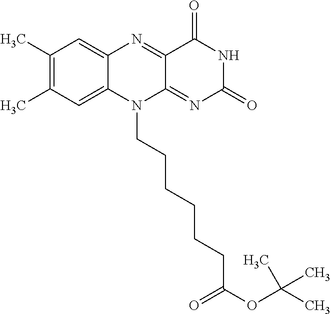

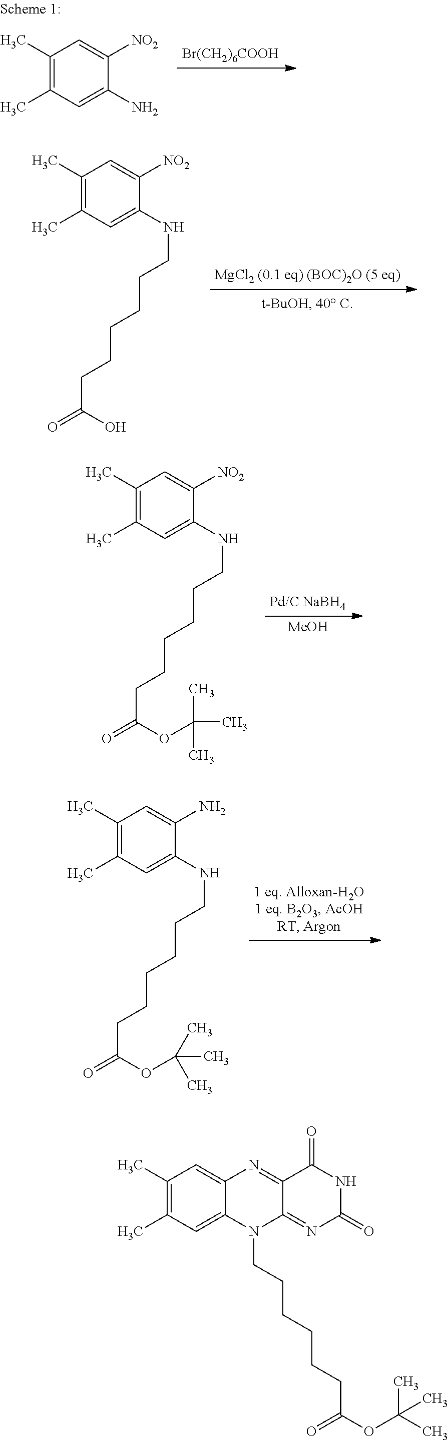

### Step 1 Preparation of 7-(4,5-Dimethyl-2-nitro-phenylamino)-heptanoic acid

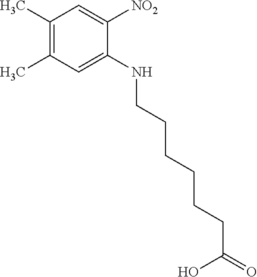

4,5-Dimethyl-2-nitro-phenylamine (2.4 g, 14 mmol) is dissolved in DMF (40 mL) and stirred at 0° C. Sodium hydride (0.57 g, 14 mmol) is added and the reaction mixture is warmed to rt. After 30 min, the mixture is cooled to 0° C. at which point a solution of 7-bromoheptanoic acid (1.0 g, 4.8 mmol) in DMF (2 mL) is added dropwise. The mixture is then allowed to warm to rt slowly over 4 h. The reaction mixture is stirred with water (20 mL) for 30 min then concentrated under vacuum. The solid is then dry loaded on silica gel and ISCO flash column purification is performed using 0 to 10% methanol in DCM over 15 minutes as the mobile phase to afford the crude product as an orange oil. Purification using preparative HPLC Method 1 afforded 7-(4,5-dimethyl-2-nitro-phenylamino)-heptanoic acid (506 mg) as an orange oil (yield: 49%). LC-MS m/z 295.0 [M+H], retention time=3.94 min.

### Step 2: Preparation of 7-(4,5-Dimethyl-2-nitro-phenylamino)-heptanoic acid tert-butyl ester

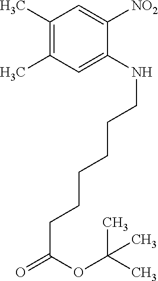

7-(4,5-Dimethyl-2-nitro-phenylamino)-heptanoic acid (2.7 g, 0.0092 mol) is dissolved in t-BuOH (40 mL) with stirring at 40° C. tert-Butoxycarbonyl anhydride (10.04 g, 0.046 mol) and magnesium chloride (0.09 g, 0.0009 mol) are added and the reaction mixture is kept under argon and allowed to stir at 40° C. for 48 h. The mixture is lyophilized and the crude is dissolved in water and extracted with ethyl acetate. The organic layer is dried with sodium sulfate and concentrated to give 3.1 g of an orange oil (crude yield=97%). LC-MS m/z 350.9 [M+H]⁺, retention time 7.66 min.

### Step 3: Preparation of 7-(2-Amino-4,5-dimethyl-phenylamino)-heptanoic acid tert-butyl ester

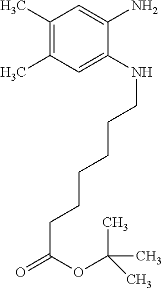

Prepared using a similar procedure to that of Example 25, step 2.

### Step 4: Preparation of 7-(7,8-Dimethyl-2,4-dioxo-3,4-dihydro-2H-benzo[g]pteridin-10-yl)-heptanoic acid tert-butyl ester

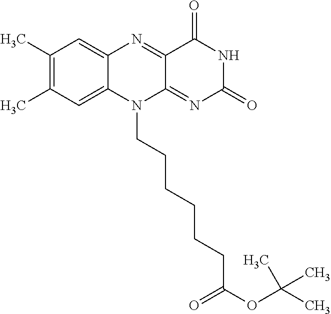

Prepared using a similar procedure to that of Example 25, step 3. Purified by preparative HPLC (Method 3). ¹H NMR (400 MHz, DMSO-d₆): δ 1.3-1.38 (m, 3H), 1.39 (s, 9H), 1.4-1.53 (m, 5H), 1.68 (m, 2H), 2.2 (t, 2H), 2.39 (s, 3H), 4.54 (m, 2H), 7.76 (s, 1H), 7.86 (s, 1H), 11.3 (s, 1H). LC-MS m/z 427.19 [M+H]⁺, retention time 8.44 min.

### Example 25

### Isopropyl 7-(7,8-dimethyl-2,4-dioxo-3,4-dihydrobenzo[g]pteridin-10(2H)-yl)heptanoate

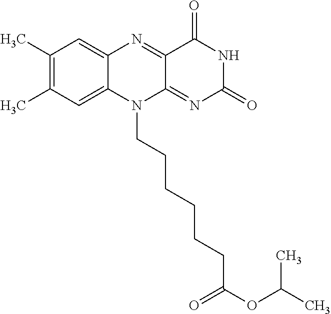

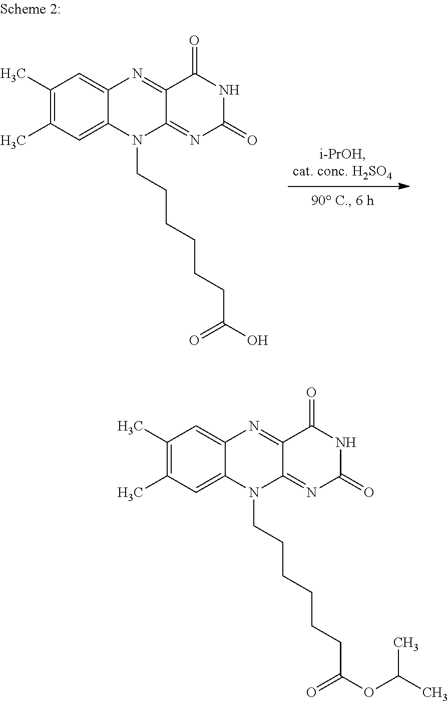

### Step 1 Preparation of 7-(4,5-Dimethyl-2-nitro-phenylamino)-heptanoic acid

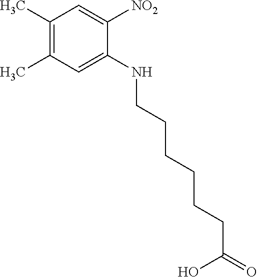

4,5-Dimethyl-2-nitro-phenylamine (2.4 g, 14 mmol) is dissolved in DMF (40 mL) and stirred at 0° C. Sodium hydride (0.57 g, 14 mmol) is added and the reaction mixture is warmed to rt. After 30 min. the mixture is cooled to 0° C. at which point a solution of 7-aminoheptanoic acid (1.0 g, 4.8 mmol) in DMF (2 mL) is added dropwise. The mixture is then allowed to warm to rt slowly over 4 h. The reaction mixture is stirred with water (20 mL) for 30 min and then concentrated under vacuum. The solid is then dry loaded onto silica gel and passed through a ISCO flash column using 0 to 10% methanol in DCM over 15 minutes as the mobile phase to afford the crude product as an orange oil. This oil is then purified using preparative HPLC (method 1) to obtain 7-(4,5-dimethyl-2-nitro-phenylamino)-heptanoic acid (506 mg) as an orange oil (yield: 49%). LC-MS m/z 295.0 [M+H], retention time=3.94 min.

### Step 2 Preparation of 7-(2-Amino-4,5-dimethyl-phenylamino)-heptanoic acid

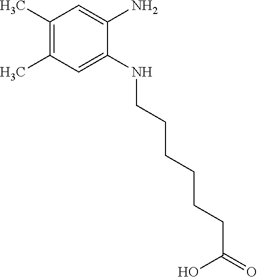

7-(4,5-Dimethyl-2-nitro-phenylamino)-heptanoic acid (70 mg, 0.24 mmol) is dissolved in methanol (5 mL) and palladium/carbon (20 mg) is added followed by sodium borohydride (91 mg, 2.4 mmol) with stirring at RT. The reaction mixture is filtered through celite after 1 h and washed with methanol. The filtrate is concentrated under vacuum to obtain the product (63 mg) as a slightly brown oil (yield: 100%).

### Step 3 Preparation of 7-(7,8-Dimethyl-2,4-dioxo-3,4-dihydro-2H-benzo[g]pteridin-10-yl)-heptanoic acid

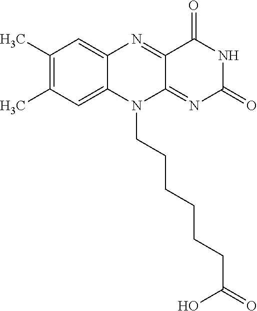

7-(2-Amino-4,5-dimethyl-phenylamino)-heptanoic acid (64 mg, 0.24 mmol) is dissolved in acetic acid (5 mL) followed by addition of boron trioxide (33 mg, 0.48 mmol) and alloxan monohydrate (38 mg, 0.24 mmol). After 3 h the reaction mixture is concentrated under vacuum and purified using preparative HPLC (method 1). 7-(7,8-Dimethyl-2,4-dioxo-3,4-dihydro-2H-benzo[g]pteridin-10-yl)-heptanoic acid (24 mg) is isolated following lyophilization of the appropriate fractions (yield: 27%). ¹H NMR (400 MHz, DMSO-d6) δ 11.37 (s, 1H), 7.92 (s, 1H), 7.83 (s, 1H), 4.55 (m, 2H), 2.40 (s, 3H), 2.19 (m, 2H), 1.68 (m, 2H), 1.47 (m, 4H), 1.33 (m, 2H). MS m/z 369.3 [M−H]⁻, retention time=6.12 min.

### Step 4 Preparation of Isopropyl 7-(7,8-dimethyl-2,4-dioxo-3,4-dihydrobenzo[g]pteridin-10(2H)-yl)heptanoate

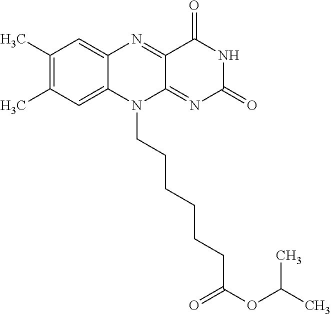

To a solution of 7-(7,8-dimethyl-2,4-dioxo-3,4-dihydrobenzo[g]pteridin-10(2H)-yl) heptanoic acid (from step 3) (1 g, 2.70 mmol) is added anhydrous isopropanol (60 mL) followed by concentrated sulfuric acid (0.5 mL). The reaction mixture is stirred at 90° C. for 6 h. The reaction mixture is neutralized with solid sodium bicarbonate, filtered and concentrated under vacuum at 25° C., and the residue is dry loaded onto silica gel using DCM as a solvent and purified by column chromatography (silica) using gradient elution (from 0% to 50% acetone in DCM). The desired product is isolated by evaporation, under vacuum at 25° C., of the fractions following of lyophilization (966 mg, 86% yield). ¹H NMR (400 MHz, CDCl₃) δ: 1.25 (d, 6H), 1.46 (m, 2H), 1.58 (m, 2H), 1.68 (m, 2H), 1.89 (m, 2H), 2.31 (t, 2H), 2.48 (s, 3H), 2.59 (s, 3H), 4.71, (m, 2H), 5.02 (m, 1H), 7.43 (s, 1H), 8.09 (s, 1H), 8.43 (s, 1H). ESI(+) m/z=413.1.

### Example 26

### ((2-(2-(7,8-dimethyl-2,4-dioxo-3,4-dihydrobenzo[g]pteridin-10(2H)-yl)acetamido)ethyl)phosphoryl)bis(oxy)bis(methylene) bis(2,2-dimethylpropanoate)

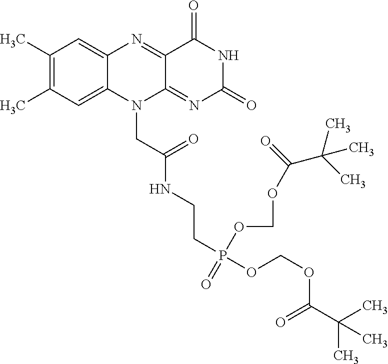

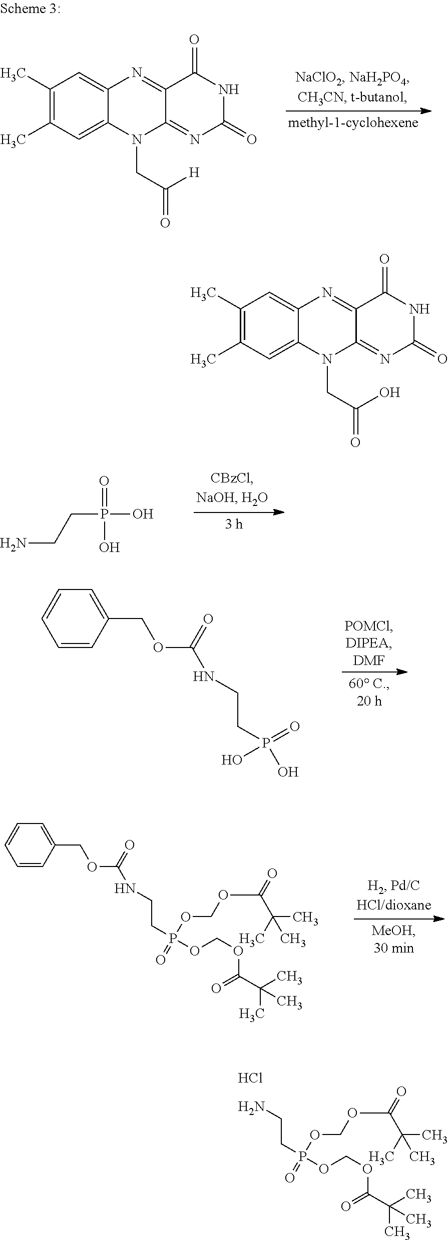

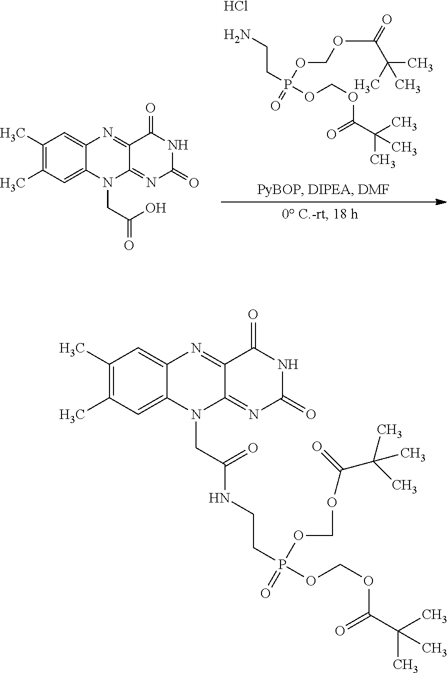

### Step 1 Preparation of (7,8-Dimethyl-2,4-dioxo-3,4-dihydro-2H-benzo[g]pteridin-10-yl)-acetic acid

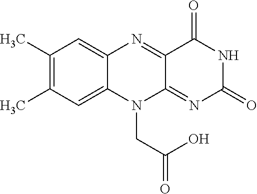

To a suspension of (7,8-dimethyl-2,4-dioxo-3,4-dihydro-2H-benzo[g]pteridin-10-yl)-acetaldehyde (prepared by the method of Example 14) (50 mg, 0.18 mmol) in acetonitrile (2 ml), tert-butanol (8 mL), and methyl-1-cyclohexene (3 mL) at 0° C., a solution of sodium chlorite (122 mg, 1.35 mmol) and sodium dihydrogen phosphate (148 mg, 1.23 mmol) in water (2 mL) is added dropwise over 5 min. After 2 h, the reaction mixture is diluted with water and the organic layer is discarded. The aqueous phase is concentrated under vacuum and the resultant crude mixture is purified via preparative HPLC. (7,8-Dimethyl-2,4-dioxo-3,4-dihydro-2H-benzo[g]pteridin-10-yl)-acetic acid (36 mg) is isolated following lyophilization of the appropriate fractions (yield: 68%). LC-MS m/z 301.1 [M+H], retention time=1.68 min.

### Step 2 Preparation of 2-(benzyloxycarbonylamino)ethylphosphonic acid

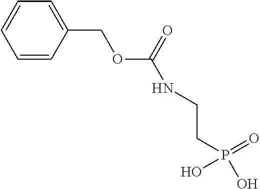

To a stirred solution of sodium hydroxide (5.76 g, 14.4 mmol) in water (40 mL) is added aminoethyl phosphonic acid (6 g, 4.8 mmol). Benzyl chloroformate (10.64 g, 6.2 mmol) is added in several portions controlling the temperature at approximately 40° C. The reaction mixture is stirred at RT for 3 h. The mixture is extracted twice with diethyl ether (2×20 mL) to remove benzyl chloroformate. The aqueous layer is acidified with concentrated HCl to pH 1-2. A white solid crashed out. The solid is collected by filtration and washed with acetonitrile. The solid is dissolved in methanol (20 mL) and treated with Dowex H⁺ resin (16 g) that has been prewashed with water and methanol. The mixture is stirred at rt for 2 h, filtered and rinsed with methanol. The filtrate is concentrated to obtain 2-(benzyloxycarbonylamino)ethylphosphonic acid (8.7 g) as a thick oil (yield 70%). LC-MS m/z 259.9 [M+H], retention time=1.76 min.

### Step 3 Preparation of ((2-(benzyloxycarbonylamino)phosphoryl)bis(oxy)bis (methylene)bis(2,2-dimethylpropanoate)

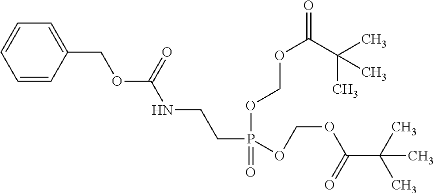

To a stirred solution of 2-(benzyloxycarbonylamino)ethylphosphonic acid (4.3 g, 1.6 mmol) in anhydrous DMF (20 mL), is added DIPEA (6.4 g, 4.9 mmol) followed by chloromethyl pivalate (7.5 g, 4.9 mmol). The reaction mixture is stirred at 60° C. for 20 h. The reaction mixture is concentrated in vacuo, the residue is diluted with water (20 mL) followed by extraction with ethyl acetate (3×25 mL). The combined organic fractions are dried over sodium sulfate and filtered. The filtrate is concentrated to obtain ((2-(benzyloxycarbonylamino)phosphoryl)bis(oxy)bis(methylene)bis(2,2-dimethylpropanoate) (7.2 g) (yield: 29%). LC-MS m/z 488.0 [M+H], retention time=4.94 min.

### Step 4 Preparation of ((2-aminoethyl)phosphoryl)bis(oxy)bis(methylene)bis(2,2-dimethylpropanoate) hydrochloride

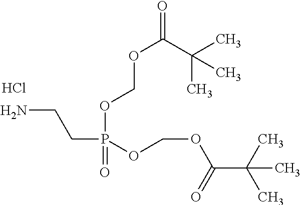

A solution of ((2-(benzyloxycarbonylamino)phosphoryl) bis(oxy)bis(methylene)bis(2,2-dimethylpropanoate) (2.01 g, 4.12 mmol) in methanol (100 ml) is purged with argon for 10 min. Then, 4N HCl/dioxane (0.8 mL, 4.94 mmol) is added followed by palladium/carbon (500 mg) and the reaction mixture is placed under an atmosphere of hydrogen for 30 min. The reaction mixture is filtered through a celite pad and the filtrate is concentrated under reduced pressure to dryness. The residue is redissolved in DCM (5 mL). Hexanes are added to precipitate out the desired product which is filtered to obtain ((2-aminoethyl)phosphoryl)bis(oxy)bis(methylene)bis(2,2-dimethylpropanoate)hydrochloride (1.41 g, 88%). LC-MS m/z 354.0 [(M−HCl)+H], retention time=2.82 min.

### Step 5 Preparation of ((2-(2-(7,8-dimethyl-2,4-dioxo-3,4-dihydrobenzo[g]pteridin-10(2H)-yl)acetamido)ethyl)phosphoryl)bis(oxy)bis(methylene)bis(2,2-dimethylpropanoate)

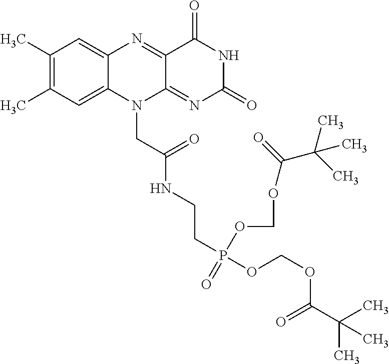

A suspension of (7,8-dimethyl-2,4-dioxo-3,4-dihydro-2H-benzo[g]pteridin-10-yl)-acetic acid (560 mg, 1.86 mmol) (from step 1) and ((2-aminoethyl)phosphoryl)_{b} is (oxy)bis(methylene)-bis(2,2-dimethylpropanoate) hydrochloride (from step 4) (799 mg, 2.05 mmol) in DMF (35 mL), is stirred at 0° C. and purged with argon for 10 min and DIPEA (0.65 mL, 3.73 mmol) is added to the reaction mixture. PyBOP (874 mg, 1.68 mmol) is added in three portions (at 30 min intervals) while maintaining the reaction mixture at 0° C. The reaction mixture is allowed to warm to room temperature and stirred for 18 h. The reaction mixture is concentrated and the residue is diluted with water (20 mL) and extracted with ethyl acetate (3×25 mL). The combined organic fractions are dried over sodium sulfate and filtered. The filtrate is concentrated and the crude is purified by silica gel chromatography (ISCO) using 100% DCM to 10% MeOH/DCM as eluent to obtain ((2-(2-(7,8-dimethyl-2,4-dioxo-3,4-dihydrobenzo[g]pteridin-10(2H)-yl)acetamido)ethyl)phosphoryl)bis(oxy)bis(methylene)bis-(2,2-dimethylpropanoate) (209 mg, 18%). ¹H NMR (400 MHz, CDCl₃) δ: 1.24 (s, 18H), 2.12 (m, 2H), 2.45 (s, 3H), 2.56 (s, 3H), 3.58 (m, 2H), 5.48 (s, 2H), 5.64 (m, 4H), 7.56 (t, 1H), 7.58 (s, 1H), 8.03 (s, 1H), 8.98 (s, 1H); LC-MS m/z 636.1 [M+H], retention time=5.36 min.

### Example 27

### 2-(2-(7,8-Dimethyl-2,4-dioxo-3,4-dihydrobenzo[g]pteridin-10(2H)-yl)acetamido)ethylphosphonic acid

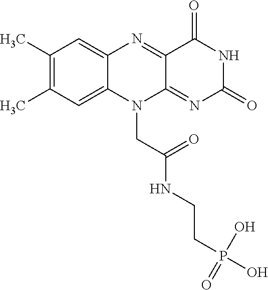

### Step 1 Preparation of diethyl 242-(7,8-dimethyl-2,4-dioxo-3,4-dihydrobenzo[g]pteridin-10(2H)-yl)acetamido)ethylphosphonate

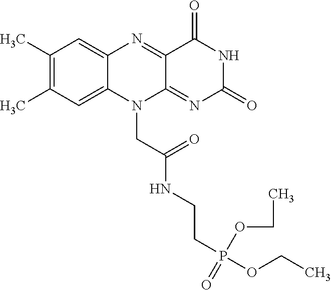

A suspension of (7,8-dimethyl-2,4-dioxo-3,4-dihydro-2H-benzo[g]pteridin-10-yl)-acetic acid (23 mg, 0.077 mmol) and diethyl 2-aminoethylphosphonate (27.9 mg, 0.154 mmol) in DMF (1 mL), is stirred at rt under argon for 10 min and DIPEA (0.1 mL) is added to the reaction mixture. HATU (58.5 mg, 0.154 mmol) is added and the mixture is stirred for 18 h at rt. Water (6 mL) is added to dissolve the mixture and the mixture was filtered and purified using preparative HPLC (method 1) to give the desired product (6.5 mg). LC-MS m/z 464.0 [M+H], retention time=2.22 min.

### Step 2 Preparation of 2-(2-(7,8-dimethyl-2,4-dioxo-3,4-dihydrobenzo[g]pteridin-10(2H)-yl)acetamido)ethylphosphonic acid

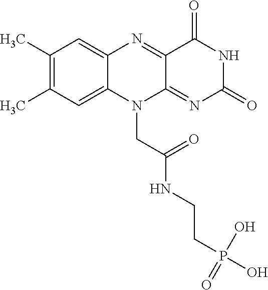

Diethyl 2-(2-(7,8-dimethyl-2,4-dioxo-3,4-dihydrobenzo[g]pteridin-10(2H)-yl)acetamido)ethylphosphonate is deprotected using a similar procedure to that of Example 19 using bromotrimethylsilane to give 2-(2-(7,8-dimethyl-2,4-dioxo-3,4-dihydrobenzo[g]pteridin-10(2H)-yl)acetamido)ethylphosphonic acid. LC-MS m/z 406.1 [M−H]⁻, retention time=3.52 min.

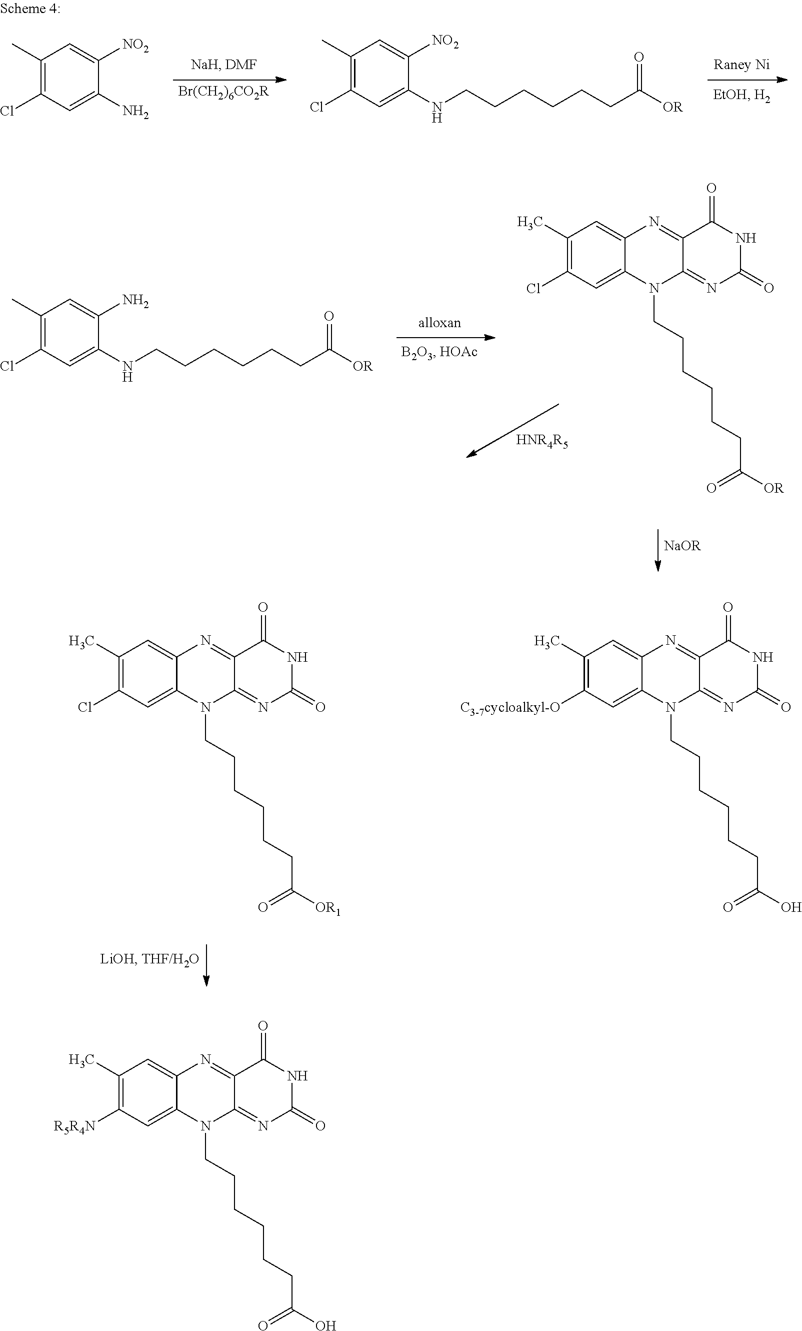

### Example 28

### Ethyl 7-(8-chloro-7-methyl-2,4-dioxo-3,4-dihydrobenzo[g]pteridin-10(2H)-yl)heptanoate

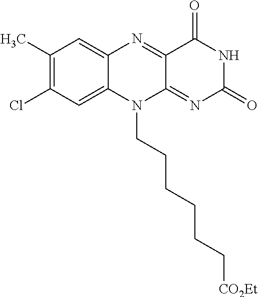

### Step 1 Preparation of Ethyl 7-(5-chloro-4-methyl-2-nitro-phenylamino)-heptanoate

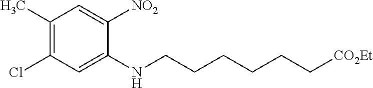

A solution of 4-amino-2-chloro-5-nitrotoluene (0.507 g, 2.72 mmol) in dry DMF (10 mL, 0.2 mol) is cooled at 0° C. under nitrogen and sodium hydride (109 mg, 60%, 2.72 mmol) is added as a solid. Hydrogen evolution was observed and the mixture is allowed to warm to rt and stirred for 30 min. Ethyl 7-bromoheptanoate (0.635 mL, 3.26 mmol) is then added drop wise via syringe and stirring continued at rt for 3 h. The reaction was concentrated in vacuo to remove DMF and the residue partitioned between DCM and saturated ammonium chloride. The layers are separated, the aqueous is extracted with DCM, and the organics are combined, dried with anhydrous sodium sulfate and concentrated. Chromatography on silica gel (Whatman, 230-400 mesh, 90 g, elution with 5% EtOAc/hexane) gave 605 mg (65%) of desired product as an orange oil. ¹H NMR (400 MHz, CDCl₃) δ 1.26 (3H, t), 1.43 (4H, m), 1.65 (2H, m), 1.73 (2H, m), 2.28 (3H, s), 2.32 (2H, t), 3.25 (2H, m), 4.13 (2H, q), 6.86 (1H, s), 7.88 (1H, m), 8.04 (1H, s); MS (ESI+) for C₁₆H₂₃ClN₂O₄ m/z 343.2 (M+H)+; HPLC retention time: 9.55 min. (System A).

### Step 2 Preparation of Ethyl 7-(2-Amino-5-chloro-4-methyl-phenylamino)-heptanoate

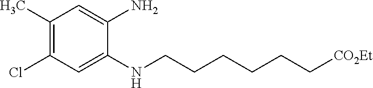

A solution of ethyl 7-(5-chloro-4-methyl-2-nitro-phenylamino)-heptanoate (64.0 mg, 0.19 mmol) in ethanol (4.0 mL) is stirred at rt and activated (wet) Raney Nickel (6.4 mg, 0.11 mmol) is added. The reaction is placed under an atmospheric pressure of hydrogen, evacuated & purged (4×) and stirred at rt. After complete (1 h), the reaction was diluted with ethyl acetate, filtered through Celite and concentrated to give 55 mg (94%) of desired product as a clear colorless oil. ¹H NMR (400 MHz, CDCl₃) δ 1.26 (3H, t), 1.41 (4H, m), 1.66 (4H, m), 2.22 (3H, s), 2.31 (2H, t), 3.04 (2H, t), 3.24 (3H, br. s.), 4.13 (2 H, q), 6.56 (1H, s), 6.60 (1H, s); MS (ESI+) for C₁₆H₂₅ClN₂O₂ m/z 313.2 (M+H)+; HPLC retention time: 6.55 min. (System A).

### Step 3 Preparation of Ethyl 7-(8-Chloro-7-methyl-2,4-dioxo-3,4-dihydrobenzo[g]pteridin-10(2H)-yl)heptanoate

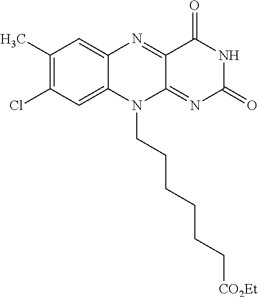

To a well-stirred solution of ethyl 7-(2-amino-5-chloro-4-methyl-phenylamino)-heptanoate (260.0 mg, 0.83 mmol) in acetic acid (4.0 mL) at rt under nitrogen is added alloxan (133.0 mg, 0.83 mmol) and boron oxide (62.53 mg, 1.7 mmol). The reaction is heated at 60° C. for 3 h, cooled to rt and concentrated. The residue is partitioned between saturated aqueous sodium bicarbonate solution and DCM (50 mL each) and extracted with DCM (3×25 mL). The organics are combined, dried with anhydrous sodium sulfate and is concentrated. Chromatography of the residue on silica gel (Silicycle, 230-400 mesh prepack, 40 g, elution with 10, 15 and 20% acetone/DCM) provided 151 mg (43%) of the desired product as a yellow solid. ¹H NMR (400 MHz, CDCl₃) δ 1.26 (3H, t), 1.44 (2H, m), 1.56 (2H, m), 1.67 (2H, m), 1.87 (2H, m), 2.33 (2 H, t), 2.57 (3H, s), 4.13 (2H, q), 4.64 (2H, m), 7.65 (1H, s), 8.18 (1H, s), 8.67 (1H, br s.); MS (ESI+) for C₂₀H₂₃ClN₄O₄ m/z 419.1 (M+H)+; HPLC retention time: 5.71 min. (System B).

### Example 29

### 7-(8-Chloro-7-methyl-2,4-dioxo-3,4-dihydrobenzo[g]pteridin-10(2H)-yl)heptanoic acid

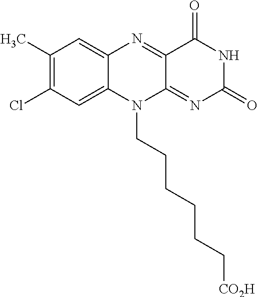

To a well-stirred slurry of ethyl 7-(8-chloro-7-methyl-2,4-dioxo-3,4-dihydrobenzo[g]pteridin-10(2H)-yl)heptanoate (35 mg, 0.084 mmol) in THF (2.0 mL, 25 mmol) at rt is added 1.0 M LiOH in water (1.0 mL). The reaction is allowed to stir for 3 h and is quenched with acetic acid (57 μL, 1.0 mmol). The mixture is allowed to stir for 1 h, the precipitate collected, washed with water and air dried to give 21 mg (64%) of the desired product as an amorphous yellow solid. ¹H NMR (400 MHz, DMSO-d6) δ 1.34 (2 H, m), 1.45 (2H, m), 1.51 (2H, m), 1.67 (2H, m), 2.21 (2H, t), 4.53 (2H, t), 8.11 (1H, s), 8.13 (1H, s), 11.39 (1H, s), 11.99 (1H, br s.); MS (ESI+) for C₁₈H₁₉ClN₄O₄ m/z 391.1 (M+H)+; HPLC retention time: 4.70 min. (System B).

### Example 30

### 7-(8-Methoxy-7-methyl-2,4-dioxo-3,4-dihydrobenzo[g]pteridin-10(2H)-yl)heptanoic acid

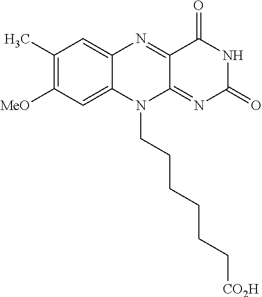

A 25 mL sealed tube is charged with ethyl 7-(8-chloro-7-methyl-2,4-dioxo-3,4-dihydrobenzo[g]pteridin-10(2H)-yl)heptanoate (51 mg, 0.12 mmol), sodium methoxide, 25% wt in MeOH (0.5 mL) and methanol (3.0 mL). The reaction is sealed under dry nitrogen and heated at 80° C. for 18 h. The reaction is cooled, concentrated, re-suspended in water and acidified with acetic acid (0.1 mL, 2 mmol). The precipitate that forms is collected and air dried to provide 25 mg (53%) of desired product as a yellow solid. ¹H NMR (400 MHz, DMSO-d6) δ 1.36 (2H, m), 1.46 (2H, m), 1.52 (2H, m), 1.73 (2H, m), 2.21 (2H, t), 2.30 (3H, s), 4.09 (3H, s), 4.65 (2H, br s), 7.16 (1H, s), 7.92 (1 H, s), 11.23 (1H, s), 11.99 (1H, s); MS (ESI+) for C₁₉H₂₂N₄O₅ m/z 387.1 (M+H)+. HPLC retention time: 4.44 min. (System B).

### Example 31

### 7-[8-(2-Hydroxy-ethylamino)-7-methyl-2,4-dioxo-3,4-dihydro-2H-benzo[g]pteridin-10-yl]-heptanoic acid

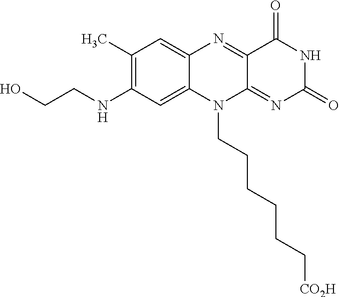

### Step 1 Preparation of Ethyl 7-8-[(2-hydroxyethyl)amino]-7-methyl-2,4-dioxo-3,4-dihydrobenzo[g]pteridin-10(2H)-ylheptanoate

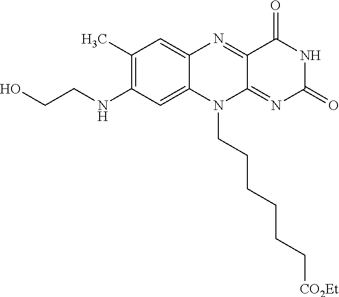

To a well-stirred slurry of ethyl 7-(8-chloro-7-methyl-2,4-dioxo-3,4-dihydrobenzo[g]pteridin-10(2H)-yl)heptanoate (81.0 mg, 0.193 mmol) in dry DMF (3.0 mL) at rt under nitrogen is added ethanolamine (0.115 mL, 1.91 mmol). The reaction is heated at 60° C. for 18 h, cooled to rt and concentrated in vacuo. The red residue is subjected to preparative reverse phase chromatography to give 31 mg (36%) of desired product as a red amorphous solid. ¹H NMR (400 MHz, DMSO-d6) δ 1.16 (3H, t), 1.35 (2 H, m), 1.442 (2H, m), 1.54 (2H, m), 1.70 (2H, m), 2.28 (5H, m), 3.52 (2H, m), 3.68 (2 H, t), 4.03 (2H, q), 4.54 (2H, m), 6.59 (1H, s), 7.10 (1H, m), 7.66 (1H, s), 10.95 (1H, s); MS (ESI+) for C₂₂H₂₉N₅O₅ m/z 444.2 (M+H)+; HPLC retention time: 4.31 min. (System B).

### Step 2 Preparation of 7-[8-(2-Hydroxy-ethylamino)-7-methyl-2,4-dioxo-3,4-dihydro-2H-benzo[g]pteridin-10-yl]-heptanoic Acid

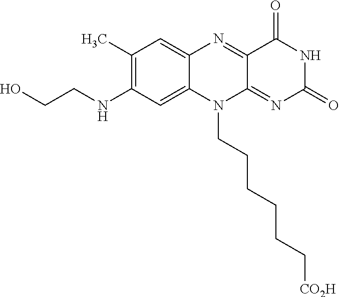

To a well stirred slurry of ethyl 7-8-[(2-hydroxyethyl)amino]-7-methyl-2,4-dioxo-3,4-dihydrobenzo[g]pteridin-10(2H)-ylheptanoate (25.0 mg, 0.06 mmol), water (1.0 mL) and THF (3.0 mL) at rt is added 1.0 M LiOH in water (0.5 mL). The reaction is stirred at rt for 1 h and quenched with acetic acid (30 uL, 0.5 mmol). This mixture is concentrated and re-suspended in water (5 mL). The precipitate is collected and dried under vacuum to give 15 mg (64%) of a red solid. ¹H NMR (400 MHz, DMSO-d₆) δ 1.35 (2H, m), 1.43 (2H, m), 1.50 (2H, m), 1.70 (2H, m), 2.20 (2H, t), 2.26 (3H, s), 3.51 (2

H, q), 3.68 (2H, t), 4.54 (2H, m), 6.59 (1H, s), 7.07 (1H, t), 7.65 (1H, s), 10.92 (1H, s); MS (ESI+) for C₂₀H₂₅N₅O₅ m/z 416.2 (M+H)+; HPLC retention time: 4.60 min. (System A).

### Example 32

### 7-[8-(2-Hydroxy-ethoxy)-7-methyl-2,4-dioxo-3,4-dihydro-2H-benzo[g]pteridin-10-yl]-heptanoic acid

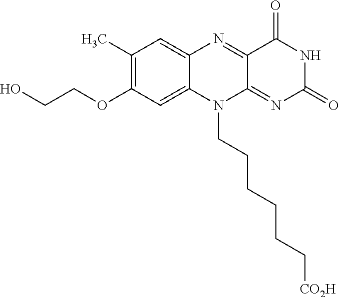

To a well-stirred slurry of sodium hydride (97.4 mg, 60%, 2.44 mmol) in dry THF (6.0 mL) at rt under dry nitrogen is added 1,2-ethanediol (0.136 mL, 2.44 mmol) slowly via syringe. Gas evolution is observed and this mixture is stirred at rt for 1 h. Solid ethyl 7-(8-chloro-7-methyl-2,4-dioxo-3,4-dihydrobenzo[g]pteridin-10(2H)-yl)heptanoate (51 mg, 0.12 mmol) is then added and this mixture is stirred at rt under dry nitrogen for 3 days. Acetic acid (0.25 mL), is added and the reaction mixture is concentrated and subjected to preparative reverse phase chromatography which provided 10 mg (20%) of desired product as a yellow solid. ¹H NMR (400 MHz, DMSO-d6) δ 1.35 (2H, m), 1.44 (2H, m), 1.51 (2H, m), 1.72 (2H, m), 2.19 (2H, m), 2.32 (3H, s), 3.84 (2 H, m), 4.34 (2H, m), 4.62 (2H, m), 5.03 (1H, br s), 7.19 (1H, s), 7.92 (1H, s), 11.24 (1 H, br s), 11.98 (1H, br s); MS (ESI+) for C₂₀H₂₄N₄O₆ m/z 417.0 (M+H)+; HPLC retention time: 4.84 min. (System A).

### Example 33

### 7-(8-Cyclopentylamino-7-methyl-2,4-dioxo-3,4-dihydro-2H-benzo[g]pteridin-10-yl)-heptanoic acid

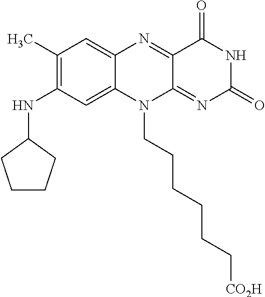

### Step 1 Preparation of ethyl 7-[8-(cyclopentylamino)-7-methyl-2,4-dioxo-3,4-dihydrobenzo[g]pteridin-10(2H)-yl]heptanoate

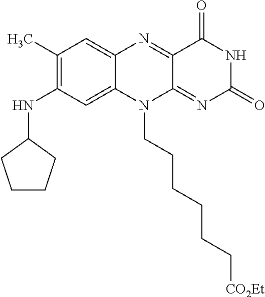

To a well-stirred slurry of ethyl 7-(8-chloro-7-methyl-2,4-dioxo-3,4-dihydrobenzo[g]-pteridin-10(2H)-yl)heptanoate (60.0 mg, 0.143 mmol) in dry DMF (4.0 mL) at rt under nitrogen is added cyclopentylamine (141 μL, 1.43 mmol). The reaction is heated at 60° C. for 18, cooled to rt and concentrated in vacuo. The red residue was subjected to preparative reverse phase chromatography to give 31 mg of a red amorphous solid that is then re-chromatographed on silica gel (ISCO Redi-Sep 12 g, elution with DCM then 1, 2, 3 and 4% MeOH/DCM) to give 10.2 mg (15%) of desired product as a red solid. ¹H NMR (400 MHz, DMSO-d6) δ 1.15 (3H, t), 1.36 (2H, m), 1.45 (2H, m), 1.54 (2H, m), 1.61 (2H, m), 1.73 (6H, m), 2.07 (2H, d), 2.28 (5H, m), 4.03 (2H, q), 4.16 (1 H, m), 4.56 (2H, br s), 6.49 (1H, s), 6.68 (1H, d), 7.65 (1H, s), 10.93 (1H, s); MS (ESI+) for C₂₅H₃₃N₅O₄ m/z 468.1 (M+H)+; HPLC retention time: 3.59 min. (System C).

### Step 2 Preparation of 7-(8-Cyclopentylamino-7-methyl-2,4-dioxo-3,4-dihydro-2H-benzo[g]pteridin-10-14)-heptanoic Acid

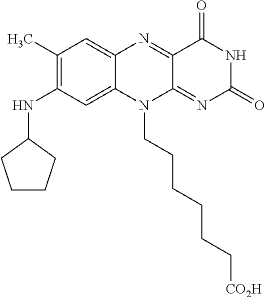

A mixture of ethyl 7-[8-(cyclopentylamino)-7-methyl-2,4-dioxo-3,4-dihydrobenzo[g]pteridin-10(2H)-yl]heptanoate (50.0 mg, 0.096 mmol) and 1.0 M LiOH in 4:1 THF/water (5 mL) is stirred at rt for 3 h. Acetic acid (0.2 mL, 4 mmol) is added and the reaction mixture is concentrated. The residue is purified by preparative reverse phase chromatography to give 16 mg (37%) of desired product as a red amorphous solid. ¹H NMR (400 MHz, DMSO-d6) δ 1.37 (2H, m), 1.48 (4H, m), 1.62 (2H, m), 1.72 (6H, m), 2.07 (2H, m), 2.20 (2H, t), 2.28 (3H, s), 4.17 (1H, m), 4.56 (2H, m), 6.49 (1H, s), 6.67 (1H, d), 7.65 (1H, s), 10.93 (1H, s), 11.99 (1H, br s); MS (ESI+) for C₂₃H₂₉N₅O₄ m/z 440.2 (M+H)+. HPLC retention time: 6.04 min. (System A).

### Example 34

### 7-8-[(2,2-Dimethylpropyl)amino]-7-methyl-2,4-dioxo-3,4-dihydrobenzo[21 pteridin-10(2H)-ylheptanoic acid

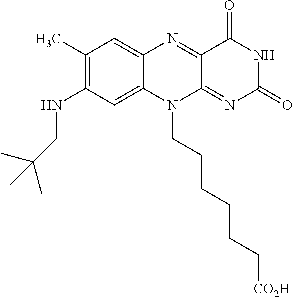

### Step 1 Preparation of Ethyl 7-84(2,2-dimethylpropyl)amino]-7-methyl-2,4-dioxo-3,4-dihydrobenzo[g]pteridin-10(2H)-ylheptanoate

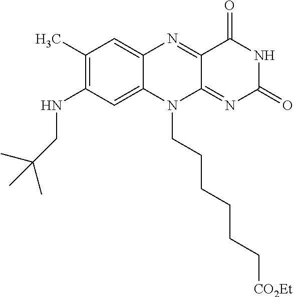

To a well-stirred slurry of ethyl 7-(8-chloro-7-methyl-2,4-dioxo-3,4-dihydrobenzo-[g]pteridin-10(2H)-yl)heptanoate (90.0 mg, 0.215 mmol) in dry DMF (6.0 mL) at rt under nitrogen is added neopentylamine (0.251 mL, 2.15 mmol). The reaction mixture is heated at 80° C. for 18 h, cooled to rt and concentrated in vacuo. The red residue was purified by chromatography on silica gel (Silicycle, 230-400 mesh, 40 g, elution with DCM then 2, and 3% MeOH/DCM) to give 45 mg (44%) of a red solid. ¹H NMR (400 MHz, CDCl₃) δ 1.12 (9 H, s), 1.26 (3H, t), 1.28 (2H, m), 1.44 (2H, m), 1.67 (2H, m), 1.87 (2H, m), 2.32 (5H, m), 3.17 (2H, d), 4.12 (2H), 4.65 (2H, m), 4.97 (1H, m), 6.41 (1H, s), 7.88 (1H, s), 8.25 (1H, s); MS (ESI+) for C₂₅H₃₅N₅O₄ m/z 470.2 (M+H)+. HPLC retention time: 3.66 min. (System C).

### Step 2 Preparation of 7-8-[(2,2-Dimethylpropyl)amino]-7-methyl-2,4-dioxo-3,4-dihydrobenzo[g]pteridin-10(2H)-ylheptanoic acid

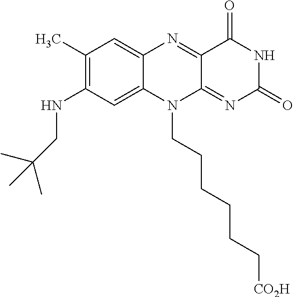

To a well stirred slurry of ethyl 7-8-[(2,2-dimethylpropyl)amino]-7-methyl-2,4-dioxo-3,4-dihydrobenzo[g]pteridin-10(2H)-ylheptanoate (29.0 mg, 0.062 mmol) and water (1.0 mL) in THF (3.0 mL) at rt was added 1.0 M LiOH in water (0.5 mL). The reaction mixture is stirred at rt for 1 h and quenched with acetic acid (50 uL, 0.09 mmol). This mixture is concentrated and re-suspended in water (5 mL). The precipitate is collected and dried under vacuum to give 24 mg (88%) of desired product as a red solid. ¹H NMR (400 MHz, DMSO-d6) δ 0.98 (9 H, s), 1.19 (2H, m), 1.35 (2H, m), 1.50 (4H, m), 1.65 (2H, m), 2.20 (2H), 2.30 (3H, s), 4.56 (2H, br. s.), 6.61 (1H, s), 6.91 (1H), 7.65 (1H, s), 10.91 (1H, s), 11.99 (1H, s); MS (ESI+) for C₂₃H₃₁N₅O₄ m/z 442.1 (M+H)+. HPLC retention time: 3.01 min. (System C).

### Example 35

### 7-[8-(Cyclopropylamino)-7-methyl-2,4-dioxo-3,4-dihydrobenzo[g]pteridin-10(2H)-yl]heptanoic acid

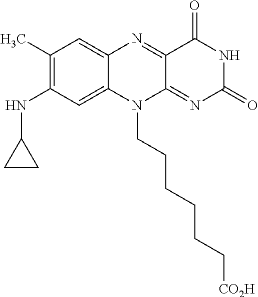

### Step 1 Preparation of Ethyl 7-[8-(Cyclopropylamino)-7-methyl-2,4-dioxo-3,4-dihydrobenzo[g]pteridin-10(2H)-yl]heptanoate

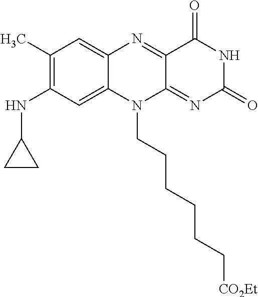

To a well-stirred slurry of ethyl 7-(8-chloro-7-methyl-2,4-dioxo-3,4-dihydrobenzo-[g]pteridin-10(2H)-yl)heptanoate (72 mg, 0.17 mmol) in dry DMF (3.1 mL) at rt under nitrogen is added cyclopropylamine (0.119 mL, 1.72 mmol). The reaction is heated at 60° C. for 24 h, cooled to rt and concentrated in vacuo. Chromatography of the residue on silica gel (Silicycle, 230-400 mesh, 40 g, elution with DCM then 2 and 3% MeOH/DCM) provides 31 mg (41%) of desired material as a red solid. ¹H NMR (400 MHz, CDCl₃) δ 0.75 (2H, m), 1.04 (2H, m), 1.25 (3H), 1.54 (2H, m), 1.60 (2H, br s), 1.66 (2H, m), 1.91 (2H, m), 2.27 (3H, s), 2.31 (2H), 2.72 (1H, m), 4.12 (2H), 4.68 (2 H, m), 6.90 (1H, s), 7.86 (1H, s), 8.34 (1H, s); MS (ESI+) for C₂₃H₂₉N₅O₄ m/z 440.1 (M+H)+. HPLC retention time: 3.01 min. (System C).

### Step 2 Preparation of 748-(Cyclopropylamino)-7-methyl-2,4-dioxo-3,4-dihydrobenzo[g]pteridin-10(21-1)-1711heptanoic acid

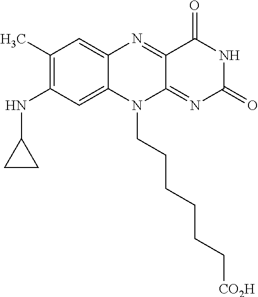

To a well stirred slurry of ethyl 7-[8-(cyclopropylamino)-7-methyl-2,4-dioxo-3,4-dihydrobenzo[g]pteridin-10(2H)-yl]heptanoate (27.1 mg, 0.062 mmol) and water (1.0 mL) in THF (3.0 mL) at rt was added 1.0 M LiOH in water (0.5 mL). The reaction is stirred at rt for 1 h and acetic acid (50 uL, 0.9 mol) is then added. The mixture is concentrated and re-suspended in water (5 mL). The precipitate is collected and dried under vacuum to give 18 mg (70%) of desired product as a red solid. ¹H NMR (400 MHz, DMSO-d6) δ 0.66 (2H, m), 0.92 (2H, m), 1.38 (2H, m), 1.52 (4H, m), 1.76 (2H, m), 2.20 (2H, m), 2.23 (3H, s), 2.71 (1H, br s), 4.54 (2H, br s), 6.88 (1H, s), 7.43 (1H, s), 7.66 (1H, s), 10.97 (1H, s), 12.00 (1H, br s); MS (ESI+) for C₂₁H₂₅N₅O₄ m/z 412.1 (M+H)+. HPLC retention time: 2.58 min. (System D).

### Example 36

### tert-Butyl 7-[8-(Cyclopropylamino)-7-methyl-2,4-dioxo-3,4-dihydrobenzo[g]pteridin-10(2H)-ylheptanoate

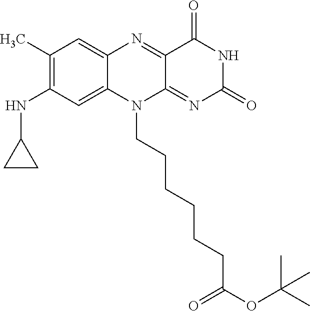

### Step 1 Preparation of ten-Butyl 74(5-chloro-4-methyl-2-nitrophenyl)amino]-heptanoate

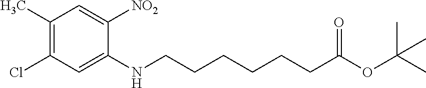

A solution of 4-amino-2-chloro-5-nitrotoluene (0.762 g, 4.1 mmol) in dry DMF (20 mL) is cooled at 0° C. under nitrogen and sodium hydride (163 mg, 60%, 4.1 mmol) is added as a solid. Hydrogen evolution is observed and the mixture is allowed to warm to rt and is stirred for 30 min. tert-Butyl 7-bromoheptanoate (1.30 g, 0.00490 mol) is then added dropwise via syringe and stirring is continued at rt for 6 h. The reaction is concentrated in vacuo and the residue is partitioned between DCM and saturated, aqueous ammounium chloride (100 mL each). The layers are separated, the aqueous extracted with DCM, and the organics combined, dried with anhydrous sodium sulfate and concentrated. Chromatography on silica gel (Silicycle, 230-400 mesh, 350 g, elution with 5-10% EtOAc/hexane) provides 1.52 g (62%) of desired product as an orange oil. ¹H NMR (400 MHz, CDCl₃) δ 1.41 (9 H, 3), 1.43 (4H, m), 1.61 (2H, m), 1.73 (2H, m), 2.23 (2H, t), 2.28 (3H, s), 3.25 (2H, m), 6.86 (1H, s), 7.89 (1H, m), 8.04 (1H, s); MS (ESI+) for C₁₈H₂₇ClN₂O₄ m/z 393.1 (M+Na)+. HPLC retention time: 6.14 min. (System D).

### Step 2 Preparation of tert-Butyl 7-[(2-Amino-5-chloro-4-methylphenyl)amino]-heptanoate

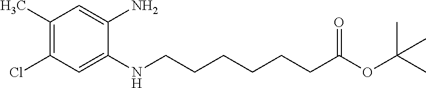

A well-stirred slurry of tert-butyl 7-[(5-chloro-4-methyl-2-nitrophenyl)amino]heptanoate (940.0 mg, 2.53 mmol) and Raney nickel (80 mg, 1.0 mol) in ethanol (15 mL) are exposed to 1 atm of hydrogen (balloon) for 6 h. The reaction is colorless when complete. The mixture is filtered through Celite and the filtrate concentrated in vacuo to provide 864 mg (98%) of the desired product as an oil. ¹H NMR (400 MHz, CDCl₃) δ 1.38 (2H, m), 1.44 (9 H, s), 1.63 (4H, m), 2.22 (3H, s), 2.23 (2H, m), 3.04 (2H), 3.23 (3H, m), 6.56 (1H, s), 6.60 (1H, s); MS (ESI+) for C₁₈H₂₇ClN₂O₂ m/z 341.1 (M+H)+. HPLC retention time: 3.98 min. (System D).

### Step 3 Preparation of tert-Butyl 7-(8-chloro-7-methyl-2,4-dioxo-3,4-dihydrobenzo[g]pteridin-10(2H)-yl)heptanoate

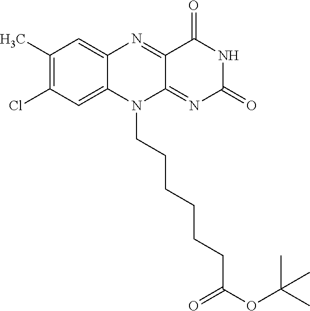

To a well-stirred mixture of tert-butyl 7-[(2-amino-5-chloro-4-methylphenyl)amino]heptanoate (860.0 mg, 2.52 mmol) and boron oxide (0.019 mg, 5.04 mmol) in acetic acid (10 mL) at rt under nitrogen is added alloxan (404 mg, 2.52 mmol) and the reaction is heated at 60° C. for 30 min. The residue is partitioned between DCM and saturated, aqueous sodium bicarbonate solution (100 mL each) and the layers are separated. The aqueous layer is extracted with DCM (3×30 mL) and the organics are combined, dried and concentrated. Chromatography of the residue on silica gel (Silicycle, 230-400 mesh, 150 g, elution with 2 and 4% EtOH in chloroform) provides 328 mg (29%) of the desired product as an amorphous yellow solid. ¹H NMR (400 MHz, DMSO-d6) δ 1.33 (2H, m), 1.38 (9 H, s), 1.44 (2H, m), 1.50 (2H, m), 1.66 (2H, m), 2.18 (2H), 4.53 (2H), 8.10 (1H, s), 8.13 (1H, s), 11.39 (1H, s); MS (ESI+) for C₂₂H₂₇ClN₄O₄ m/z 447.0 (M+H)+; HPLC retention time: 4.16 min. (System D)

### Step 4 Preparation of tert-Butyl 7-[-(cyclopropylamino)-7-methyl-2,4-dioxo-3,4-dihydrobenzo[g]pteridin-10(2H)-yl]heptanoate (I) and tert-Butyl 7-[8-(dimethylamino)-7-methyl-2,4-dioxo-3,4-dihydrobenzo[g]pteridin-10(2H)-yl]heptanoate (II)

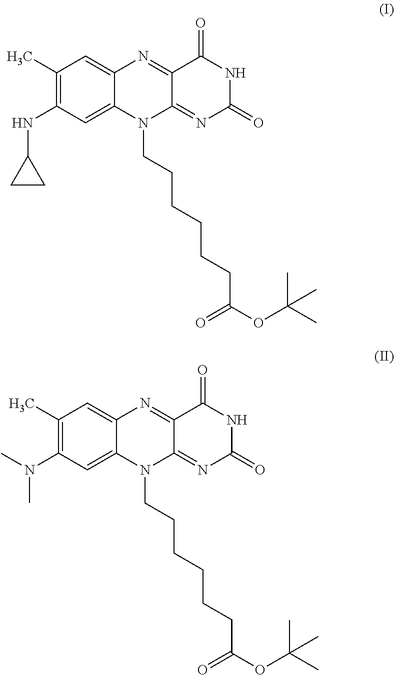

To a well-stirred slurry of tert-butyl 7-(8-chloro-7-methyl-2,4-dioxo-3,4-dihydrobenzo[g]pteridin-10(2H)-yl)heptanoate (51.0 mg, 0.114 mmol) in dry DMF (3.0 mL) at rt under nitrogen is added cyclopropylamine (0.10 mL, 1.4 mmol). The reaction is heated at 60° C. for 18 h, cooled to rt and concentrated in vacuo. The red residue is purified by chromatography on silica gel (Silicycle, 230-400 mesh, 40 g, elution with DCM then 2, 3 then 4% MeOH/DCM) to give 27 mg (50%) of tert-butyl 7-[8-(cyclopropylamino)-7-methyl-2,4-dioxo-3,4-dihydrobenzo pteridin-10(2H)-yl]heptanoate (I) as a red solid. ¹H NMR (400 MHz, DMSO-d6) δ 0.66 (2H, m), 0.91 (2 H, m), 1.33 (2H, m), 1.36 (9 H, s), 1.50 (4H, m), 1.75 (2H, m), 2.18 (2H2.23 (3H, s), 2.72 (1H, m), 4.54 (2H, m), 6.88 (1H, s), 7.43 (1H, s), 7.66 (1H, s), 10.97 (1H, s); MS (ESI+) for C₂₅H₃₃N₅O₄ m/z 468.1 (M+H)+; HPLC retention time: 3.63 min. (System C).

In addition, 23 mg (44%) of tert-butyl 7-[8-(dimethylamino)-7-methyl-2,4-dioxo-3,4-dihydrobenzo[g]pteridin-10(2H)-yl]heptanoate (II) was isolated from this column as the result of dimethylamine being present or generated during the reaction. ¹H NMR (400 MHz, CDCl₃) δ 1.44 (2H, m), 1.45 (9 H, s), 1.55 (2H, m), 1.62 (2H, m), 1.89 (2H, m), 2.23 (2H), 2.49 (3H, s), 3.12 (6H, s), 4.65 (2H, m), 6.70 (1H, s), 7.95 (1H, s), 8.29 (1H, br s); MS (ESI+) for C₂₄H₃₃N₅O₄ m/z 456.1 (M+H)+. HPLC retention time: 3.74 min. (System C).

### Example 37

### 4-[2-(7,8-Dimethyl-2,4-dioxo-3,4-dihydrobenzo[g]pteridin-10(2H)-yl)ethyoxy]butanoic acid

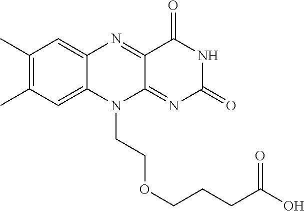

### Step 1 Preparation of tert-Butyl 4-{2-[(4,5-dimethyl-2-nitrophenyl)amino]ethoxy}butanoate

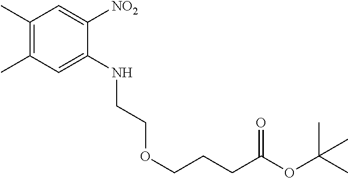

A solution of 1-bromo-4,5-dimethyl-2-nitrobenzene (0.163 g, 0.708 mmol) and tert-butyl 4-(2-aminoethoxy)butanoate [prepared using a procedure similar to that described in EP0655439A2 (1994)] in DMSO (1.4 mL) is heated at 100° C. under an atmosphere of nitrogen. After 20 hours, the orange solution is partitioned between ethyl acetate and aqueous sodium bicarbonate. The organic layer is washed twice more with aqueous sodium bicarbonate and then brine; it is dried with sodium sulfate, filtered, and concentrated in vacuo. The red residue is flash chromatographed on a 20×80 mm silica gel column eluted with 50% chloroform/hexanes and 100% chloroform, then 5% methanol/chloroform. The methanol/chloroform fraction is chromatographed again, eluted with 5% ethyl acetate/hexanes and 10% ethyl acetate/hexanes to give 0.11 g of desired product as an orange oil. (Yield: 44%). ¹H NMR (400 MHz, CDCl₃) δ 8.16 (br s, 1H), 7.95 (s, 1H), 6.67 (s, 1H), 3.72 (t, 2H), 3.55 (t, 2H), 3.49 (m, 2H), 2.35 (t, 2H), 2.29 (s, 3H), 2.20 (s, 3H), 1.91 (m, 2H), 1.46 (s, 9 H). HPLC retention time 5.30 min. (System D).

### Step 2 Preparation of tert-Butyl 4-(2-(7,8-dimethyl-2,4-dioxo-3,4-dihydrobenzo[g]pteridin-10(2H)-yl)ethoxy)butanoate (I) and 4-(2-(7,8-Dimethyl-2,4-dioxo-3,4-dihydrobenzo[g]pteridin-10(2H)-yl)ethoxy)butanoic acid (II)

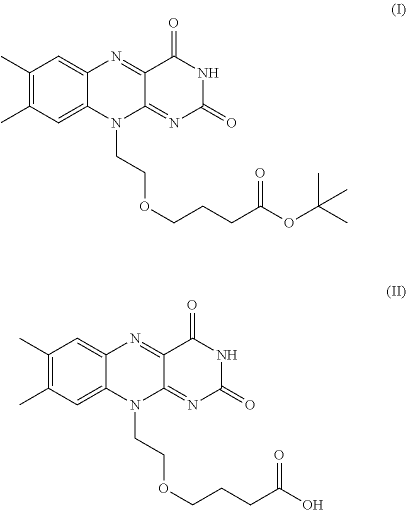

A well-stirred slurry of Raney nickel (25 mg, 0.43 mmol) and tert-butyl 4-{2-[(4,5-dimethyl-2-nitrophenyl)aminoethoxy}butanoate (0.110 g, 0.312 mmol) in ethanol (3 mL) is alternately evacuated then covered with 1 atm hydrogen (3×) (balloon). After stirring overnight at rt, the mixture is filtered through Celite®, and concentrated then azeotroped with toluene to give a brown oil, 0.117 g. The brown oil intermediate is dissolved in acetic acid (3 mL) and alloxan monohydrate (50.0 mg, 0.312 mmol) is added. The mixture is heated at 80° C. under an atmosphere of nitrogen and after an hour, diboron trioxide (43.4 mg, 0.624 mmol) is added and heating continued for 3 hours at 80° C. and 60° C. for another 13 hours. The reaction mixture consisted of both the tert-butyl ester and the corresponding carboxylic acid. The reaction was concentrated in vacuo and the residue is flash chromatographed on a 28×80 mm silica gel column, eluted with 1% to 5% methanol/methylene chloride and 5% of (10% formic acid in methanol)/methylene chloride (200 mL) and 10% methanol/methylene chloride (200 mL). Fractions with product were combined and concentrated in vacuo to give 18 mg of tert-butyl 4-(2-(7,8-dimethyl-2,4-dioxo-3,4-dihydrobenzo[g]pteridin-10(2H)-yl)ethoxy)butanoate (I) as an amorphous orange solid. (Yield: 13%). ¹H NMR (400 MHz, DMSO-d₆) δ 11.33 (s, 1H), 7.89 (s, 1H), 7.88 (s, 1H), 4.79 (m, 2H), 3.79 (t, 2H), 3.38 (t, 2H), 2.50 (s, 3H), 2.41 (s, 3H), 2.07 (t, 2H), 1.59 (m, 2H), 1.35 (s, 9 H). Mass spec (ESI+) for C₂₂H₂₈N₄O₅ m/z 429.1 (M+H)+. HPLC retention time 3.52 min. (System D).

Late, impure chromatography fractions were subjected to preparative plate silica chromatography (20×20 cm plates silica gel 60 F254, 0.5 mm thickness, eluted with 10% ethanol/chloroform). The appropriate yellow band is removed, slurried with methanol/dichloromethane, and filtered through Celite®. Solvent is removed in vacuo to give 6.4 mg of 4-(2-(7,8-dimethyl-2,4-dioxo-3,4-dihydrobenzo[g]pteridin-10(2H)-yl)ethoxy)butanoic acid (II) as an amorphous yellow solid. (Yield: 5.5%). ¹H NMR (400 MHz, DMSO-d₆) δ 11.32 (br s, 1H), 7.89 (s, 2H), 4.79 (t, 2H), 3.78 (t, 2H), 3.39 (m, 2H), 2.50 (s, 3H), 2.40 (s, 3H), 2.05 (t, 2H), 1.61 (m, 2H). Mass spec (ESI+) for C₁₈H₂₀N₄O₅ m/z 373.0 (M+H)+. HPLC retention time 2.38 min. (System D).

### Example 38

### Methyl 4-(2-(7,8-dimethyl-2,4-dioxo-3,4-dihydrobenzo[g]pteridin-10(2H)-yl)ethoxy)butanoate

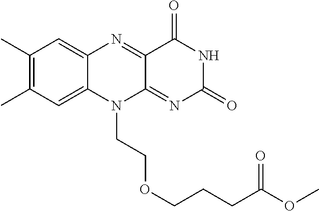

tert-Butyl-4-(2-(7,8-dimethyl-2,4-dioxo-3,4-dihydrobenzo[g]pteridin-10(2H)-yl)ethoxy)butanoate (11 mg, 0.026 mmol) was dissolved in methanol (5 mL) and cooled on an ice bath. Methanol (5.0 mL,) was cooled in an ice bath and acetyl chloride (1.0 mL, 14 mmol) was added. After 10 minutes, it was added to the above solution and the ice bath was removed. After stirring 2 days, concentration in vacuo gave brown oil. Preparative plate silica chromatography (20×20 cm plates silica gel 60 F254, 0.5 mm thickness), eluted with 10% ethanol/chloroform gave 6.1 mg yellow solid. (Yield: 61%). ¹H NMR (400 MHz, DMSO-d₆) δ 11.33 (s, 1H), 7.89 (s, 1H), 7.87 (s, 1H), 4.78 (t, 2H), 3.78 (t, 2H), 3.53 (s, 3H), 3.39 (m, 2H), 2.49 (s, 3H), 2.40 (s, 3H), 2.18 (t, 2H), 1.64 (m, 2H). Mass spec (ESI+) for C₁₉H₂₂N₄O₅ m/z 387.1 (M+H)+. HPLC retention time 2.78 minutes (System D).

### Example 39

### 7-Methyl-2,4-dioxo-10-((2S,3S,4R)-2,3,4,5-tetrahydroxypentyl)-2,3,4,10-tetrahydrobenzo[g]pteridine-8-carbonitrile

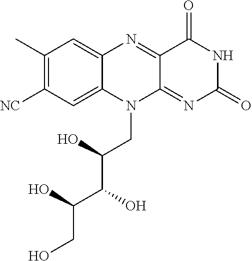

Prepared using a procedure similar to that described in The Journal of Biological Chemistry (1998), 273, 8975-8982, Yerramilli V. S, N. Murthy and Vincent Massey. ¹H NMR (400 MHz, D₂O) δ 8.31 (s, 1H), 8.01 (s, 1H), 4.78 (m, 2H), 4.28 (m, 1H), 3.86 (m, 1H), 3.78 (m, 1H), 3.75 (m, 1H), 3.61 (m, 1H), 2.57 (m, 3H). Mass spec (ESI+) for C₁₇H₁₇N₅O₆ m/z 388.1 (M+H)+, 410.1 (M+Na)+. HPLC retention time 1.98 min. (System D).

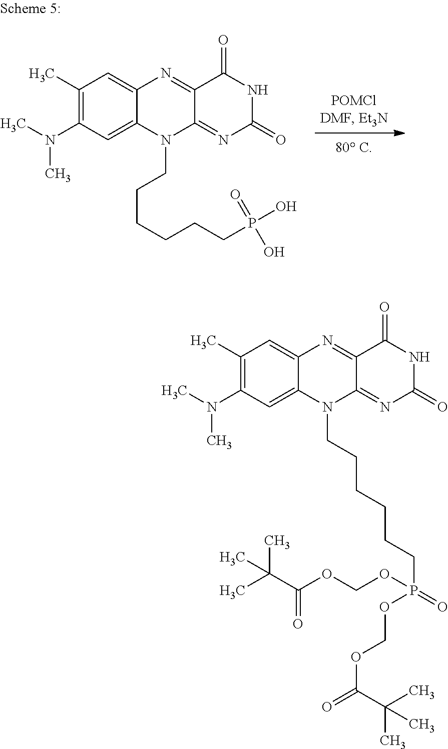

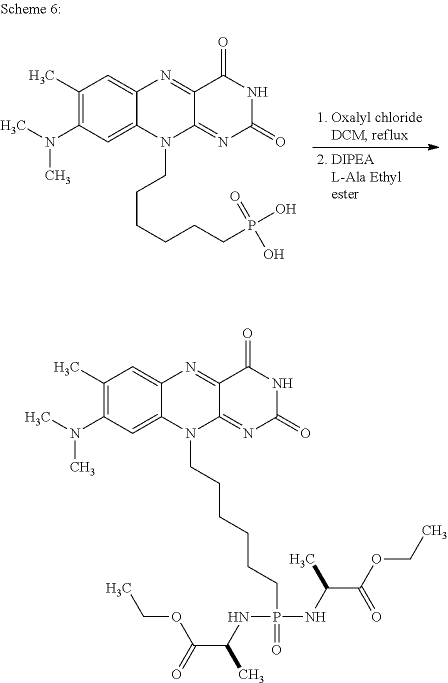

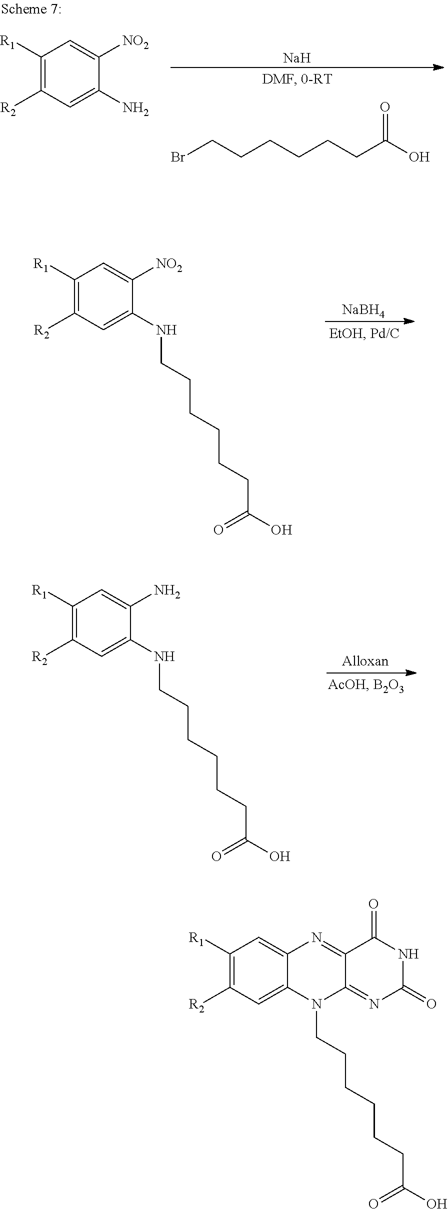

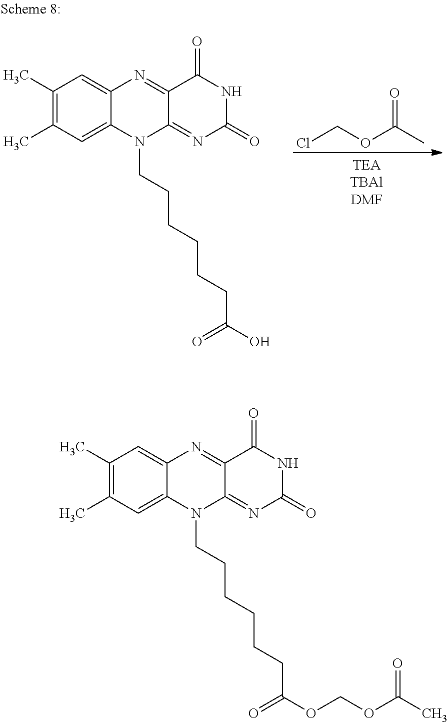

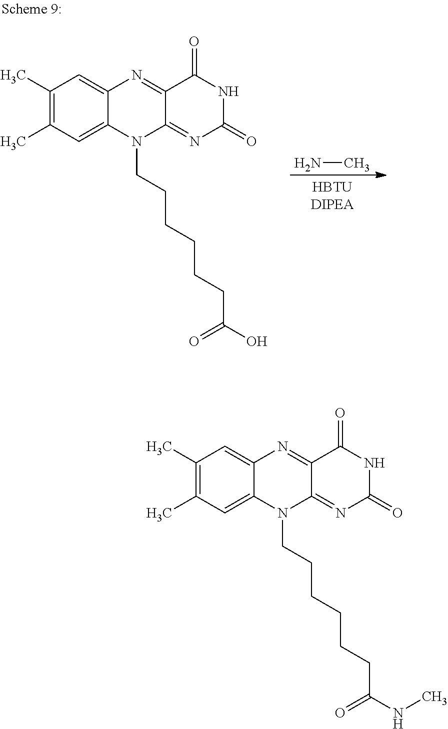

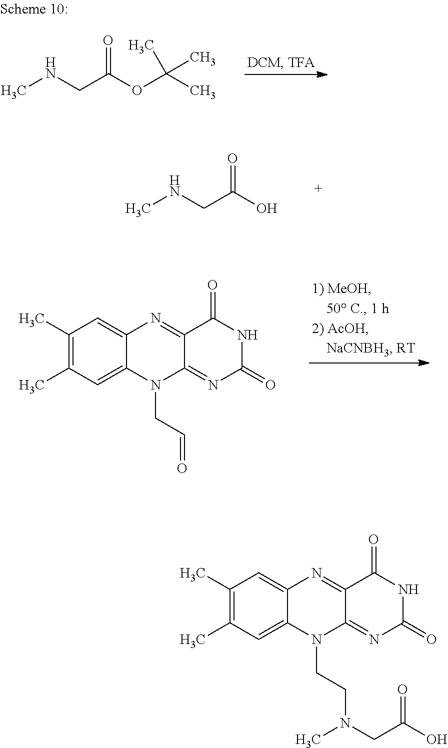

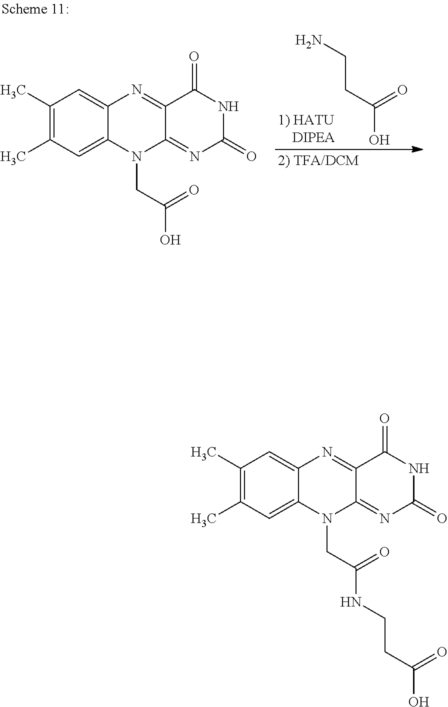

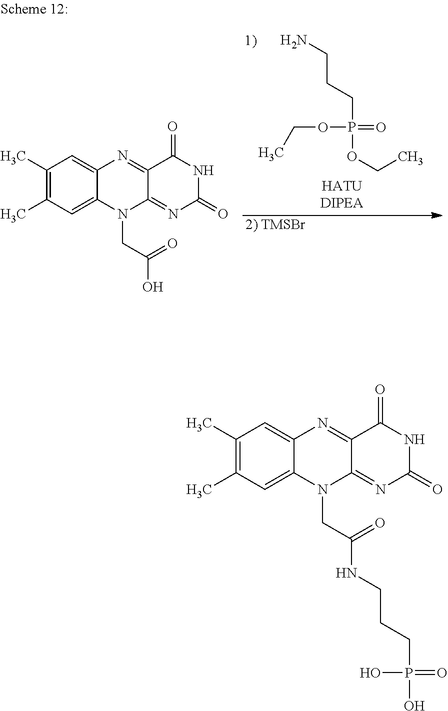

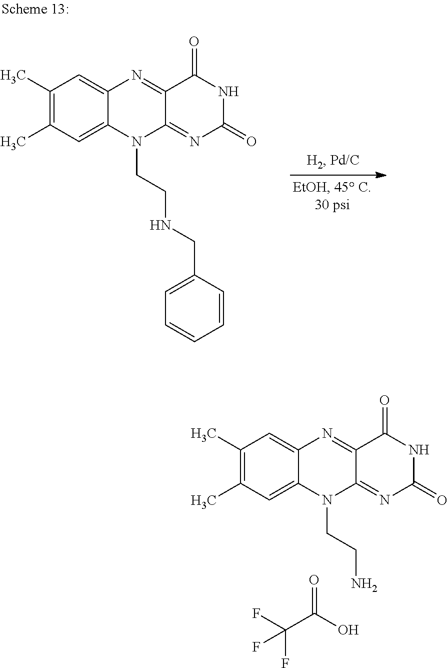

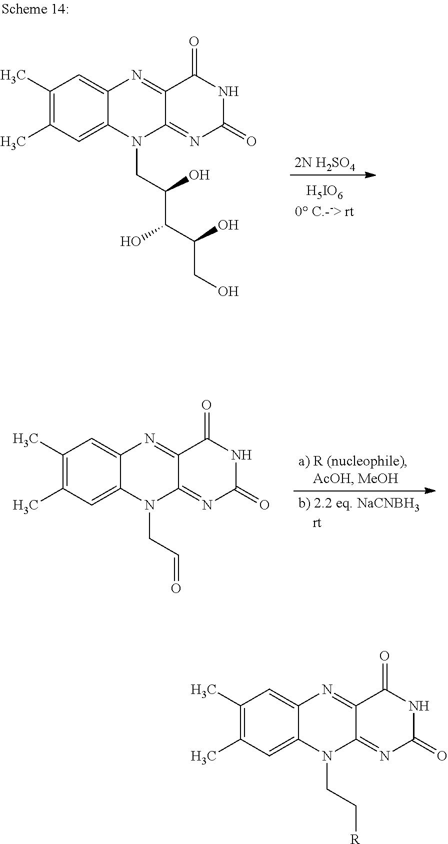

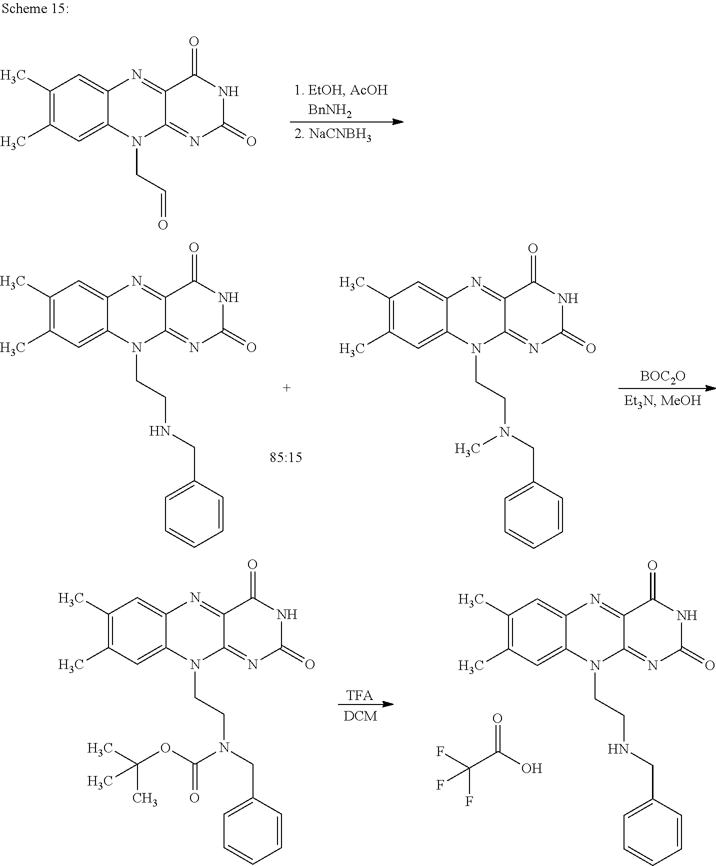

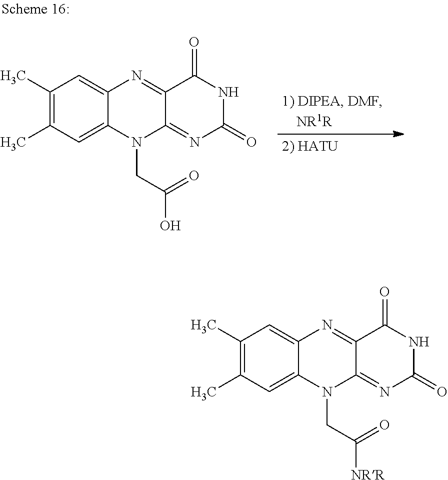

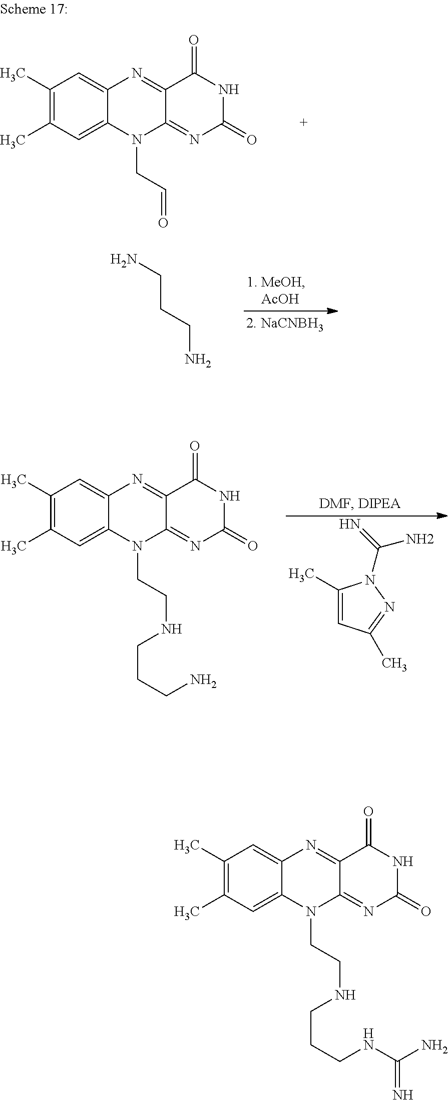

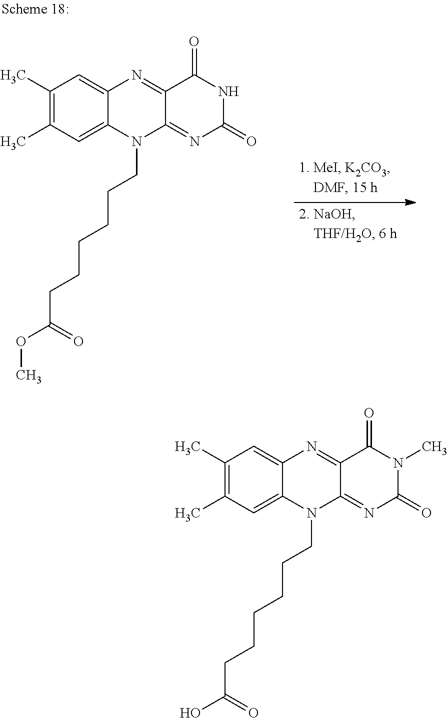

### Example 40

### 10-(2-Aminoethyl)-7,8-dimethylbenzo[g]pteridine-2,4(3H,10H)-dione-2,2,2-trifluoroacetate salt

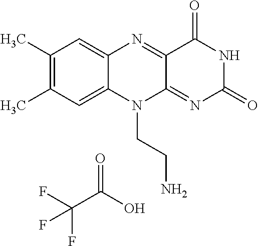

A solution of 10-(2-(benzylamino)ethyl)-7,8-dimethylbenzo[g]pteridine-2,4(3H,10H)-dione (see Example 55 for preparation) (395 mg, 1.05 mmol) and Pd/C (75 mg) in absolute EtOH (100 ml) is stirred under an atmosphere of hydrogen at 30 psi and 45° C. overnight. The mixture is filtered through a celite pad. The filtrate is concentrated under reduced pressure to dryness to obtain a crude product (230 mg, 77%). Crude product (19.5 mg, 0.07 mmol) is dissolved in MeOH (8 ml) and purified by preparative HPLC (Method 2). Lyophilization of the combined fractions affords desired product (5.0 mg, 14%) as a brown solid. ¹H NMR (400 MHz, DMSO-d₆) δ 2.42 (s, 3H), 2.50 (s, 3H), 4.20 (m, 2H), 4.87 (m, 2H), 7.81 (s, 1H), 7.88 (m, 3H), 7.97 (s, 1H), 11.45 (s, 1H).

### Example 41

### 7-(3,7,8-Trimethyl-2,4-dioxo-3,4-dihydro-2H-benzo[g]pteridin-10-yl)-heptanoic acid

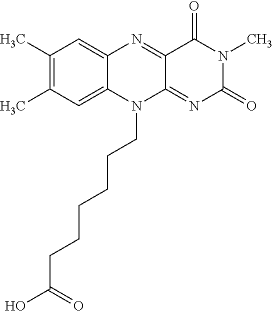

### Step 1 Preparation of 7-(4,5-Dimethyl-2-nitro-phenylamino)-heptanoic acid

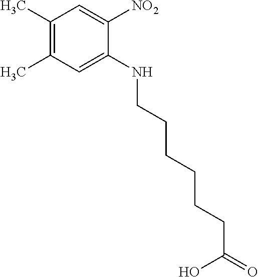

4,5-Dimethyl-2-nitro-phenylamine (2.4 g, 14 mmol) was dissolved in DMF (40 mL) and set to 0° C. with stirring and then sodium hydride (0.57 g, 14 mmol) was added and the reaction mixture was warmed to room temperature. After 30 min. the mixture was cooled to 0° C. at which point a solution of 7-aminoheptanoic acid (1.0 g, 4.8 mmol) in DMF (2 mL) was added dropwise. The mixture is then allowed to warm to room temperature slowly over 4 h. The reaction mixture is stirred with water (20 mL) for 30 min. and then concentrated under vacuum. The solid is then dry loaded onto silica gel and ISCO flash column purification is performed (0 to 10% methanol in DCM) as the mobile phase to afford the crude product as an orange oil. The crude product is then purified using preparative HPLC (Method 1) to obtain 7-(4,5-dimethyl-2-nitro-phenylamino)-heptanoic acid (506 mg) as an orange oil (Yield: 49%). LC-MS m/z 295.0 [M+H], retention time=3.94 min.

### Step 2 Preparation of 7-(2-Amino-4,5-dimethyl-phenylamino)-heptanoic acid

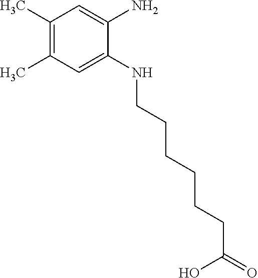

7-(4,5-Dimethyl-2-nitro-phenylamino)-heptanoic acid (70 mg, 0.24 mmol) is dissolved in methanol (5 mL) and Pd/C (20 mg) is added followed by sodium borohydride (91 mg, 2.4 mmol) with stirring. The reaction mixture is filtered through celite after 1 h and is washed with methanol. The filtrate is concentrated under vacuum to obtain the product (63 mg) as a slightly brown oil (Yield: 100%).

### Step 3 Preparation of 7-(7,8-Dimethyl-2,4-dioxo-3,4-dihydro-2H-benzo[g]pteridin-10-yl)-heptanoic acid

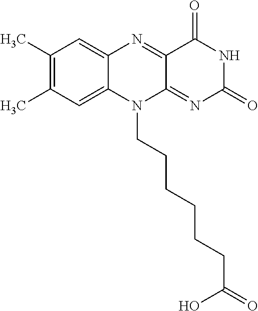

7-(2-Amino-4,5-dimethyl-phenylamino)-heptanoic acid (64 mg, 0.24 mmol) is dissolved in acetic acid (5 mL) and then boron trioxide (33 mg, 0.48 mmol) and alloxan monohydrate (38 mg, 0.24 mmol) are added. After 3 h, the reaction mixture is concentrated under vacuum and purified using preparative HPLC (Method 1). 7-(7,8-Dimethyl-2,4-dioxo-3,4-dihydro-2H-benzo[g]pteridin-10-yl)-heptanoic acid (24 mg) is isolated following lyophilization of the appropriate fractions (Yield: 27%). ¹H NMR (400 MHz, DMSO-d6) δ 11.37 (s, 1H), 7.92 (s, 1H), 7.83 (s, 1H), 4.55 (m, 2H), 2.40 (s, 3H), 2.19 (m, 2H), 1.68 (m, 2H), 1.47 (m, 4H), 1.33 (m, 2H). MS m/z 369.3 [M−H]—, retention time=6.12 min.

### Step 4 Preparation of Methyl 7-(7,8-dimethyl-2,4-dioxo-3,4-dihydrobenzo[g]pteridin-10(2H)-yl)heptanoate

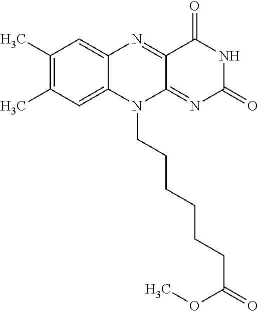

The preparation of this compound is similar to that of Example 25 using the acid from the previous step and methanol as a substitute for i-PrOH.

### Step 5 Preparation of 7-(3,7,8-Trimethyl-2,4-dioxo-3,4-dihydro-2H-benzo[g]pteridin-10-yl)-heptanoic acid methyl ester

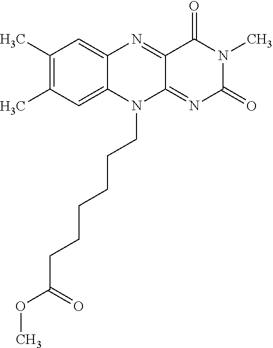

7-(7,8-Dimethyl-2,4-dioxo-3,4-dihydro-2H-benzo[g]pteridin-10-yl)-heptanoic acid methyl ester (from the previous step) (350 mg, 0.91 mmol) is dissolved in DMF (2 mL). Methyl iodide (2 mL) and K₂CO₃ (251 mg, 182 mmol) are added to the reaction mixture and the mixture is stirred at rt for 15 h. The reaction mixture is filtered and the solid is washed with MeOH (10 mL). The filtrate is evaporated to a volume of 6.5 mL. The solution is diluted with water (1 mL) and purified by preparative HPLC (Method 1). The desired product is isolated in 12% (45 mg) yield after lyophilization. This product is used in the following step without further purification.

### Step 6 Preparation of 7-(3,7,8-Trimethyl-2,4-dioxo-3,4-dihydro-2H-benzo[g]pteridin-10-yl)-heptanoic acid

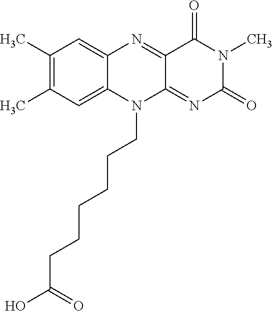

7-(3,7,8-Trimethyl-2,4-dioxo-3,4-dihydro-2H-benzo[g]pteridin-10-yl)-heptanoic acid methyl ester (24.5 mg, 0.061 mmol) is suspended in H₂O (1.5 mL) and NaOH (26 mg, 0.61 mmol) is added to this suspension. THF (0.1 mL) is added to dissolve the rest of the acid. The reaction mixture is stirred at room temperature for 6 h When the reaction is complete (as monitored by LCMS, ˜5 h), the pH of the reaction is adjusted to 1 by 1M aqueous HCl and is stirred for 30 min. The reaction mixture is filtered and purified by preparative HPLC (Method 4). After lyophilization, the desired compound (3.9 mg) is isolated in 17% yield. NMR (400 MHz, MeOD-d4) δ 1.48 (m, 2H), 1.58 (m, 2H), 1.67 (m, 2H), 1.90 (m, 2H), 2.34 (m, 2H), 2.50 (s, 3H), 2.62 (s, 3H), 3.48 (3H), 4.78 (t, 2H), 7.81 (s, 1H), 8.02 (s, 1H).

### Example 42

### ((6(8-(Dimethylamino)-7-methyl-2,4-dioxo-3,4-dihydrobenzo[g]-pteridin-10(2H)-yl)hexyl)phosphoryl)bis(oxy)bis(methylene)bis(2,2-dimethylpropanoate)

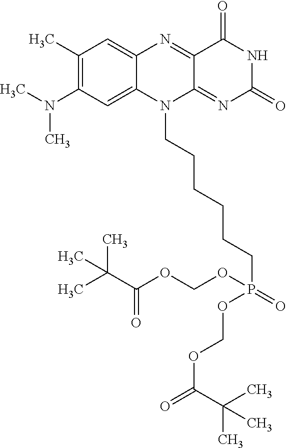

POMCl (327 mg, 2.17 mmol) is added to a suspension of 6-(8-(dimethylamino)-7-methyl-2,4-dioxo-3,4-dihydrobenzo[g]pteridin-10(2H)-yl)hexylphosphonic acid (122 mg, 0.28 mmol) and Et₃N (2 ml) in anhydrous DMF (10 ml). The reaction mixture is heated at 80° C. under an argon atmosphere for 24 h. Et₂O (20 ml) is added and the mixture is stirred at rt. The resulting solid is filtered off and the filtrate is concentrated under reduced pressure, dissolved in ACN (6 ml)/water (2 ml) and purified by preparative HPLC (Method 2). Lyophilization of combined fractions (LCMS) affords 11.4 mg (0.008 mmol) of desired product as a red solid. The product is further purified via preparative TLC (10% MeOH in DCM) to afford desired product (9.0 mg, 4.8%) as a red solid. ¹H NMR (400 MHz, DMSO-d₆) δ 1.14 (s, 18H), 1.44 (m, 6H), 1.71 (m, 2H), 1.80 (m, 2H), 2.45 (s, 3H), 3.05 (s, 6H), 4.58 (m, 2H), 5.58 (m, 4H), 6.86 (s, 1H), 7.81 (s, 1H), 11.12 (s, 1H).

### Example 43

### (2S,2′S)-Diethyl-2,2′-((6-(8-(dimethylamino)-7-methyl-2,4-dioxo-3,4-dihydrobenzo[g]pteridin-10(2H)-yl)hexyl)phosphoryl)bis(azanediyl)dipropanoate

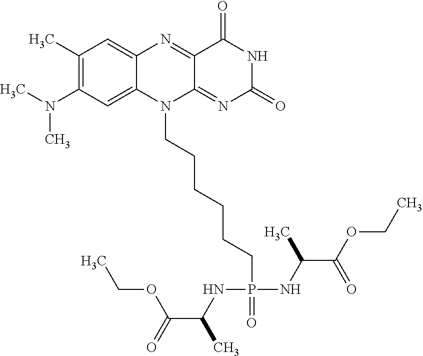

### Step 1 Preparation of 6-(8-(Dimethylamino)-7-methyl-2,4-dioxo-3,4-dihydrobenzo[g]pteridin-10(2H)-yl)hexylphosphonic acid

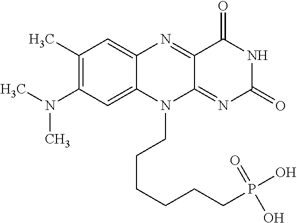

The above compound is prepared as in Example 13

### Step 2 Preparation of (2S,2′S)-Diethyl-2,2′-((6-(8-(dimethylamino)-7-methyl-2,4-dioxo-3,4-dihydrobenzo[g]pteridin-10(2H)yl)hexyl)phosphoryl)bis(azanediyl) dipropanoate

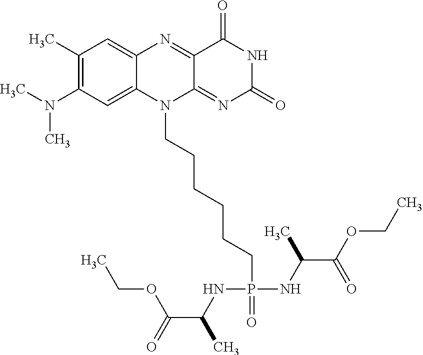

Oxalyl chloride (140 mg, 1.1 mmol) is added to a suspension of 6-(8-(dimethylamino)-7-methyl-2,4-dioxo-3,4-dihydrobenzo[g]pteridin-10(2H)-yl)hexylphosphonic acid (40 mg, 0.09 mmol) in anhydrous DCM (10 ml) followed by DMF (0.2 ml). The reaction mixture becomes homogenous and is refluxed under an argon atmosphere for 2 h. The solvent is concentrated to dryness. The resulting solid is dissolved in anhydrous DCM (10 ml), cooled to 0° C., then L-alanine ethyl ester (152 mg, 0.98 mmol) is added, followed by DIPEA (0.2 ml, 1.2 mmol). The reaction is warmed to rt with stirring for 1 h. The solution is concentrated under reduced pressure, dissolved in ACN (6 ml)/water (2 ml) and purified by preparative HPLC (Method 2). Lyophilization of combined fractions (LCMS) affords desired product (5.4 mg, 9.2%) of as a purple solid. ¹H NMR (400 MHz, MeOD-d4) δ 1.31 (m, 6H), 1.65 (m, 14H), 1.93 (m, 2H), 2.62 (d, 3H) and 2.63 (d, 3H), 3.31 (s, 6H), 4.25 (m, 4H), 4.67 (m, 2H), 4.60-4.74 (m, 1H), 4.80-5.80 (m, 1H), and 6.94 (s, 1H), 7.94 and 7.98 (s, 1H).

### Example 44

### 7(7,8-Dimethyl-2,4-dioxo-3,4-dihydro-2H-benzo[g]pteridin-10-yl)-heptanoic acid

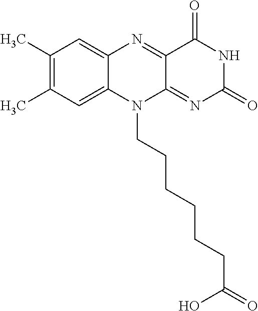

### Step 1 Preparation of 7-(4,5-Dimethyl-2-nitro-phenylamino)-heptanoic acid

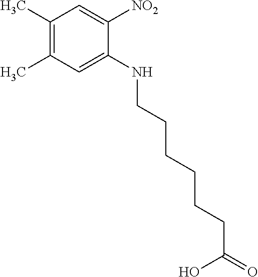

4,5-Dimethyl-2-nitro-phenylamine (2.4 g, 14 mmol) is dissolved in DMF (40 mL) and stirred at 0° C. and then sodium hydride (0.57 g, 14 mmol) is added and the reaction mixture is warmed to room temperature. After 30 min., the mixture is cooled to 0° C. at which point a solution of 7-aminoheptanoic acid (1.0 g, 4.8 mmol) in DMF (2 mL) is added dropwise. The mixture is then allowed to warm to room temperature slowly over 4 h. The reaction mixture is stirred with water (20 mL) for 30 min. then concentrated under vacuum. The solid is then dry loaded onto silicagel and ISCO flash column purification is performed (0 to 10% methanol in DCM) as the mobile phase to afford the desired product as an orange oil 7-(4,5-dimethyl-2-nitro-phenylamino)-heptanoic acid (506 mg) (Yield: 49%). LC-MS m/z 295.0 [M+H], retention time=3.94 min.

### Step 2 Preparation of 7-(2-Amino-4,5-dimethyl-phenylamino)-heptanoic acid

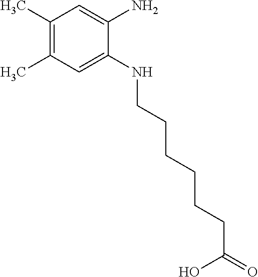

7-(4,5-Dimethyl-2-nitro-phenylamino)-heptanoic acid (70 mg, 0.24 mmol) is dissolved in methanol (5 mL) and Pd/C (20 mg) is added followed by sodium borohydride (91 mg, 2.4 mmol) with stirring. The reaction mixture is filtered through celite after 1 h and the celite is washed with methanol. The filtrate is concentrated under vacuum to obtain the desired product (63 mg) as a slightly brown oil (Yield: 100%).

### Step 3 Preparation of 7-(7,8-Dimethyl-2,4-dioxo-3,4-dihydro-2H-benzo[g]pteridin-10-yl)-heptanoic acid

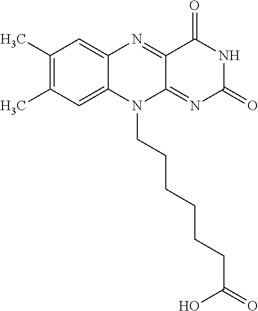

7-(2-Amino-4,5-dimethyl-phenylamino)-heptanoic acid (64 mg, 0.24 mmol) is dissolved in acetic acid (5 mL) then boron trioxide (33 mg, 0.48 mmol) and alloxan monohydrate (38 mg, 0.24 mmol) are added. After 3 h, the reaction mixture is concentrated under vacuum and purified using preparative HPLC (Method 1). 7-(7,8-Dimethyl-2,4-dioxo-3,4-dihydro-2H-benzo[g]pteridin-10-yl)-heptanoic acid (24 mg) is isolated following lyophilization of the appropriate fractions (Yield: 27%). ¹H NMR (400 MHz, DMSO-d6) δ 11.37 (s, 1H), 7.92 (s, 1H), 7.83 (s, 1H), 4.55 (m, 2H), 2.40 (s, 3H), 2.19 (m, 2H), 1.68 (m, 2H), 1.47 (m, 4H), 1.33 (m, 2H). MS m/z 369.3 [M−H]⁻, retention time=6.12 min.

### Example 41

### 7-(2,4-Dioxo-3,4-dihydrobenzo[g]pteridin-10(2H)-yl)heptanoic acid

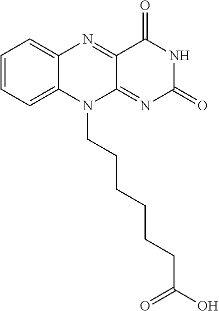

7-(2,4-Dioxo-3,4-dihydrobenzo[g]pteridin-10(2H)-yl)heptanoic acid is synthesized from 7-(2-aminophenylamino)heptanoic acid (382 mg, 1.62 mmol) and alloxan (260 mg, 1.61 mmol) following the procedure described for Example 44. The reaction mixture is concentrated under vacuum and the resulting residue is purified by column chromatography (silica gel) using gradient elution (0 to 10% MeOH in DCM). The desired product is isolated following evaporation under vacuum (288 mg, 52% yield). ¹H NMR (400 MHz, DMSO) δ 1.33 (m, 2H), 1.45 (m, 4H), 1.69 (m, 2H), 2.03 (t, 2H), 4.54 (m, 2H), 7.62 (m, 1H), 7.91 (m, 2H), 8.11 (d, 1H), 11.37 (bs, 2H); ESI(+) m/z=343.0.

### Example 46

### Acetoxymethyl 7-(7,8-dimethyl-2,4-dioxo-3,4-dihydrobenzo[g]pteridin-10(2H)-yl)heptanoate

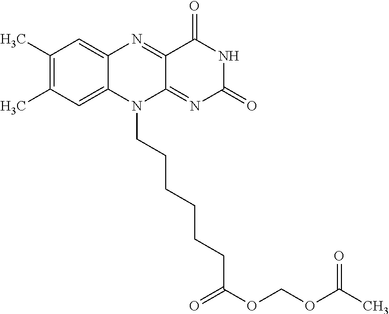

A mixture of 7-(7,8-dimethyl-2,4-dioxo-3,4-dihydrobenzo[g]pteridin-10(2H)-yl)heptanoic acid (30 mg, 0.081 mmol), chloromethylacetate (44 mg, 0.405 mmol), triethylamine (45 μL, 0.324 mmol), and tetrabutylammonium iodide (150 mg, 0.405 mmol) in 2 mL of anhydrous DMF (2 mL) were stirred at 25° C. for 24 h. The reaction mixture is concentrated under vacuum and the residue is dry loaded on silica gel using DCM as solvent and purified by BIOTAGE flash column chromatography using gradient from 1 to 5% MeOH in DCM as eluent. Desired product (11 mg) is isolated in 29% yield. ¹H NMR (400 MHz, CDCl₃) δ 1.48 (m, 2H), 1.54 (m, 2H), 1.69 (m, 2H), 1.87 (m, 2H), 2.04 (s, 3H), 2.39 (m, 2H), 2.45 (s, 3H), 2.57 (s, 3H), 4.69 (m, 2H), 5.69 (s, 2H), 7.40 (s, 1H), 8.07 (s, 1H), 8.36 (s, 1H); ESI(+) m/z=443.0.

### Example 47

### 7-(7,8-dimethyl-2,4-dioxo-3,4-dihydrobenzo[g]pteridin-10(2H)-yl)-N-methylheptanamide

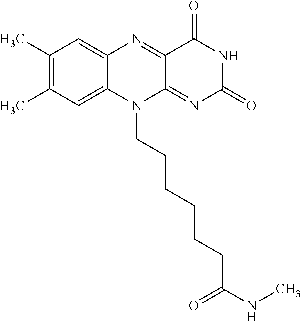

A mixture of 7-(7,8-dimethyl-2,4-dioxo-3,4-dihydrobenzo[g]pteridin-10(2H)-yl)heptanoic acid (30 mg, 0.081 mmol), HBTU (31 mg, 0.081 mmol), DIPEA (42 μL, 0.243 mmol), and 2M methylamine in THF solution (405 μL, 0.81 mmol) in 2 mL of anhydrous DMF (2 mL) were stirred at 25° C. for 16 h. The reaction mixture is concentrated under vacuum and the residue is dry loaded on silica gel and purified by BIOTAGE flash column chromatography using a gradient from 1 to 2% MeOH in DCM as eluent. Desired product (21 mg) is isolated in 68% yield. ¹H NMR (400 MHz, CDCl₃) δ 1.48 (m, 2H), 1.56 (m, 2H), 1.72 (m, 2H), 1.87 (m, 2H), 2.22 (t, 2H), 2.46 (s, 3H), 2.58 (s, 3H), 2.80 (d, 3H), 4.68 (m, 2H), 5.89 (bs, 1H), 7.42 (s, 1H), 8.07 (s, 1H) 8.41 (s, 1H); ESI(+) m/z=384.1.

### Example 48

### 2-(2-(7,8-Dimethyl-2,4-dioxo-3,4-dihydrobenzo[2]pteridin-10(2H)-yl)ethylamino)benzoic acid

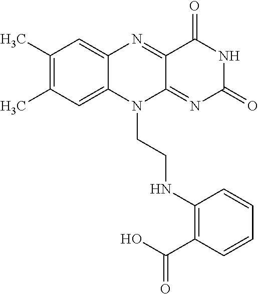

### Step 1 Preparation of tert-Butyl 2-(2-(7,8-dimethyl-2,4-dioxo-3,4-dihydrobenzo[g]pteridin-10(2H)-yl)ethylamino)

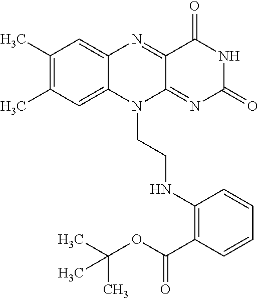

To a solution of (7,8-dimethyl-2,4-dioxo-3,4-dihydro-2H-benzo[g]pteridin-10-yl)-acetaldehyde from Example 14 (50 mg, 0.176 mmol) in methanol (5 mL) are added tert-butyl 2-aminobenzoate (37 mg, 0.193 mmol) and AcOH (75 μL) at room temperature. The reaction is stirred at 50° C. for 30 min. The reaction is cooled to room temperature and sodium cyanoborohydride (25 mg, 0.39 mmol) is added and the reaction mixture is stirred at 50° C. for 24 h. The product precipitates from the reaction mixture and is filtered and washed with MeOH (10 mL) and dried overnight under vacuum (49 mg, 61% yield). ¹H NMR (400 MHz, DMSO) δ 1.45 (s, 9H), 2.29 (s, 3H), 2.36 (s, 3H), 3.34 (bs, 1H), 3.72 (m, 2H), 4.74 (m, 2H), 6.62 (t, 1H), 7.31 (d, 1H), 7.41 (t, 1H), 7.60 (s, 1H), 7.74 (d, 1H), 7.86 (s, 1H), 11.35 (bs, 1H) ESI(+) m/z=462.0.

### Step 2 Preparation of 2-(2-(7,8-Dimethyl-2,4-dioxo-3,4-dihydrobenzo[g]pteridin-10(2H)-yl)ethylamino)benzoic acid

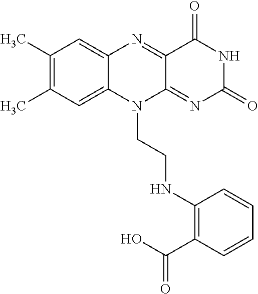

To a solution of tert-butyl 2-(2-(7,8-dimethyl-2,4-dioxo-3,4-dihydrobenzo[g]pteridin-10(2H)-yl)ethylamino)benzoate (29 mg, 0.063 mmol) in anhydrous DCM (2 mL) is added TFA (500 μL, 6.45 mmol) and the mixture is stirred at 40° C. for 16 h. The solution is concentrated under reduced pressure, and the residue is dissolved in DMSO (1 mL), filtered, and purified by preparatory HPLC (Method 1). The desired product (14.0 mg) is isolated following lyophilization (Yield: 55%). ¹H NMR (400 MHz, DMSO) δ 2.33 (s, 3H), 2.37 (s, 3H), 3.72 (m, 2H), 4.75 (m, 2H), 6.60 (t, 1H), 7.31 (d, 1H), 7.39 (m, 1H), 7.7 (s, 1H), 7.76 (dd, 1H), 7.86 (s, 1H), 11.36 (s, 1H), 12.50 (b s, 1H) ESI(+) m/z=370.0.

### Example 49

### {[2-(7,8-Dimethyl-2,4-dioxo-3,4-dihydro-2H-benzo[g]pteridin-10-yl)-ethyl]-methyl-amino}-acetic acid

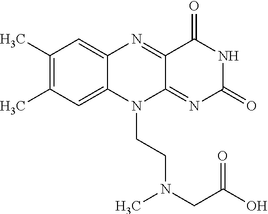

### Step 1 Preparation of 2-(Methylamino)acetic acid

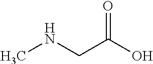

tert-Butyl 2-(methylamino)acetate (250 mg, 1.4 mmol) is dissolved in DCM (2 mL) and TFA (2 mL) is added at room temperature and stirred for 2 h. The reaction mixture is then concentrated to dryness. The residue is mixed with TEA (0.5 mL) and again concentrated to dryness to afford the product as a clear oil and is used directly in the next step.

### Step 2 Preparation of {[2-(7,8-Dimethyl-2,4-dioxo-3,4-dihydro-2H-benzo[g]pteridin-10-yl)-ethyl]-methyl-amino}-acetic acid

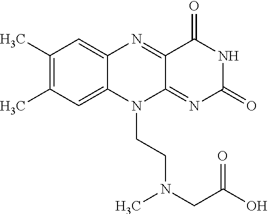

Dimethyl-2,4-dioxo-3,4-dihydro-2H-benzo[g]pteridin-10-yl)-acetaldehyde (100 mg, 0.35 mmol) from Example 14 and 2-(methylamino)acetic acid (133 mg, 1.0 mmol) are suspended in methanol (15 mL) and stirred at 50° C. After one hour, the mixture is cooled to room temperature and acetic acid (0.1 mL) is added, followed by sodium cyanoborohydride (55 mg, 0.9 mmol). The resulting mixture was stirred for 16 h. Dilute with water (3 mL) and purify via prep HPLC (Method 1) to obtain the product (62 mg) as a yellow solid (Yield: 50%). LCMS m/z 358.13 [M+H]⁺, retention time=5.13 min

### Example 50

### (S)-2-Amino-6-[2-(7,8-dimethyl-2,4-dioxo-3,4-dihydro-2H-benzo[g]pteridin-10-yl)-ethylamino]-hexanoic acid

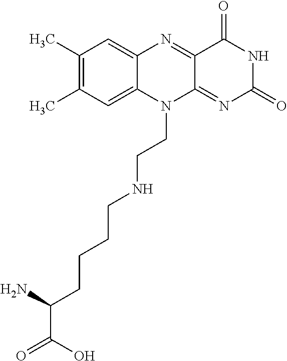

### Step 1 Preparation of (S)-2-tert-Butoxycarbonylamino-6-[2-(7,8-dimethyl-2,4-dioxo-3,4-dihydro-2H-benzo[g]pteridin-10-yl)-ethylamino]-hexanoic acid

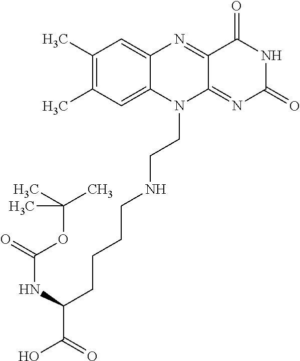

Into a suspension of 2-(7,8-dimethyl-2,4-dioxo-3,4-dihydro-2H-benzo[g]pteridin-10-yl)-acetaldehyde (68 mg, 0.239 mmol) in MeOH (2.5 mL) are added (S)-6-amino-2-tert-butoxycarbonylamino-hexanoic acid (65 mg, 0.263 mol), NaCNBH₃ (18 mg, 0.287 mmol), and AcOH (0.1 mL) respectively and stirred at room temperature for 24 h. The reaction mixture is heated at 40° C. for 16 h. The solvent is removed under vacuum and the crude is used in the following step without further purification.

### Step 2 Preparation of (S)-2-Amino-6-[2-(7,8-dimethyl-2,4-dioxo-3,4-dihydro-2H-benzo[g]pteridin-10-yl)-ethylamino]-hexanoic acid

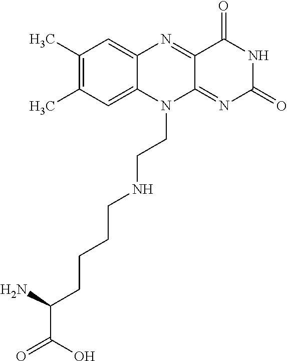

The crude product from step 1 is dissolved in TFA (2 mL) and DCM (2 mL) and stirred at room temperature for 24 h. The solvent is removed and the crude product is dissolved in H₂O (8 mL) and purified by preparative HPLC (Method 2). After lyophilization the desired compound (26 mg) is obtained in 26% yield after two steps. ¹H NMR (400 MHz, MeOH-d4) δ 1.36 (s, 2H), 1.61 (m, 2H), 1.82 (m, 2H), 2.00 (m, 2H), 2.48 (s, 3H), 2.61 (s, 3H), 3.22 (t, 2H), 3.67 (t, 2H), 3.99 (t, 1H) 5.10 (t, 2H), 7.81 (s, 1H), 7.91 (s, 1H).

### Example 51

### 2-[2-(7,8-Dimethyl-2,4-dioxo-3,4-dihydro-2H-benzo[g]pteridin-10-yl)-ethylamino]-pentanedioic acid di-tert-butyl ester

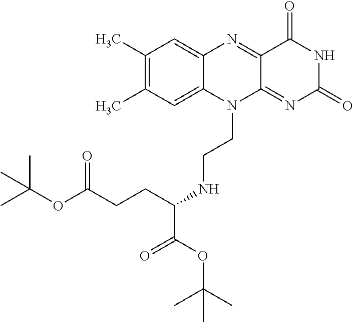

To a suspension of (7,8-dimethyl-2,4-dioxo-3,4-dihydro-2H-benzo[g]pteridin-10-yl)-acetaldehyde (see Example 14) (100 mg, 0.352 mmol) in methanol (5 mL) is added L-glutamic acid di-tert-butyl ester hydrochloride (104 mg, 0.352 mmol) at room temperature followed by glacial acetic acid (0.1 mL). The reaction mixture is heated to 40° C. and stirred for 1 h. Then sodium cyanoborohydride (49 mg, 0.775 mmol) is added, and the solution is stirred for 16 h at 20° C. The reaction mixture is concentrated; the residue is dissolved in DMF/water (1/3) and purified by preparative HPLC (Method 1). 2-[2-(7,8-Dimethyl-2,4-dioxo-3,4-dihydro-2H-benzo[g]pteridin-10-yl)-ethylamino]-pentanedioic acid di-tert-butyl ester (30 mg) is isolated following lyophilization of the appropriate fractions (Yield: 16%). ¹H NMR (400 MHz, DMSO-d6) δ 1.40 (s, 9H), 1.46 (s, 9H), 1.94 (m, 1H), 2.05 (m, 1H), 2.29 (m, 1H), 2.42 (s, 3H), 2.53 (s, 3H), 3.36 (m, 2H), 4.14 (m, 1H), 4.86 (m, 1H), 5.00 (m, 1H), 7.82 (s, 1H), 7.86 (s, 1H), 9.30 (br s, 1H), 11.46 (s, 1H).

### Example 52

### 3-[2-(7,8-Dimethyl-2,4-dioxo-3,4-dihydro-2H-benzo[g]pteridin-10-yl)-acetylamino]-propionic acid

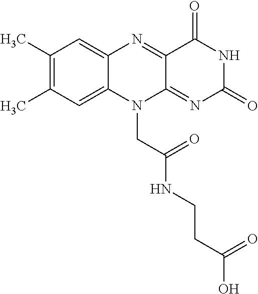

### Step 1 Preparation of 3-[2-(7,8-Dimethyl-2,4-dioxo-3,4-dihydro-2H-benzo[g]pteridin-10-yl)-acetylamino]-propionic acid tert-butyl ester

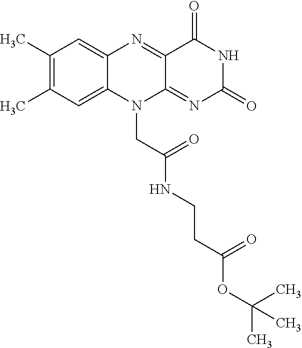

To a suspension of (7,8-dimethyl-2,4-dioxo-3,4-dihydro-2H-benzo[g]pteridin-10-yl)-acetic acid (100 mg, 0.34 mmol) and 3-amino-propionic acid tert-butyl ester (99 mg, 0.68 mmol) in DMF (3 mL), DIPEA (0.13 mL, 0.68 mmol) and HATU (128 mg, 0.68 mmol) are added sequentially at room temperature. The temperature is then increased to 40° C. and the reaction mixture is stirred for 18 h. The reaction mixture is cooled to room temperature, diluted with water (3 mL) and purified using preparative HPLC purification (Method 1). 3-[2-(7,8-Dimethyl-2,4-dioxo-3,4-dihydro-2H-benzo[g]pteridin-10-yl)-acetylamino]-propionic acid tert-butyl ester (16 mg) is isolated following lyophilization of the appropriate fractions (Yield: 11%). LC-MS m/z 428.0 [M+H]⁺. Retention time=2.75 min.

### Step 2 Preparation of 3-[2-(7,8-Dimethyl-2,4-dioxo-3,4-dihydro-2H-benzo[g]pteridin-10-yl)-acetylamino]-propionic acid

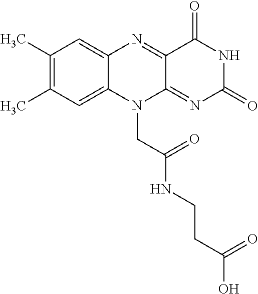

To a suspension of 3-[2-(7,8-dimethyl-2,4-dioxo-3,4-dihydro-2H-benzo[g]pteridin-10-yl)-acetylamino]-propionic acid tert-butyl ester (7 mg, 0.02 mmol) in DCM (2 mL) is added trifluoroacetic acid (2 mL) at room temperature. After 2 h, the reaction mixture is concentrated and the residual material is purified using prep HPLC (Method 1) and the appropriate fractions were lyophilized to obtain 3-[2-(7,8-dimethyl-2,4-dioxo-3,4-dihydro-2H-benzo[g]pteridin-10-yl)-acetylamino]-propionic acid (3.2 mg) as a yellow solid (Yield: 53%). ¹H NMR (400 MHz, DMSO-d6) δ 11.42 (s, 1H), 8.41 (s, 1H), 7.93 (s, 1H), 7.49 (s, 1H), 5.71 (s, 1H), 5.28 (m, 2H), 2.46 (s, 3H), 2.40 (s, 3H), MS m/z 371.9 [M+H]⁺, retention time=6.24 min.

### Example 53

### {3-[2-(7,8-Dimethyl-2,4-dioxo-3,4-dihydro-2H-benzo[g]pteridin-10-yl)-acetylamino]-propyl}-phosphonic acid

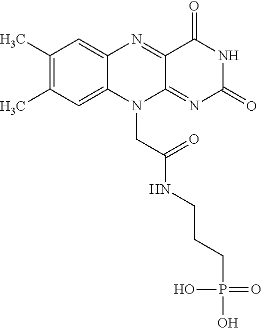

### Step 1 Preparation of {3-[2-(7,8-Dimethyl-2,4-dioxo-3,4-dihydro-2H-benzo[g]pteridin-10-yl)-acetylamino]-propyl}-phosphonic acid diethyl ester

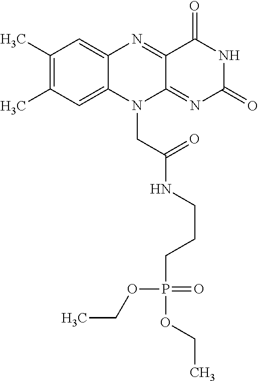

To a suspension of (7,8-dimethyl-2,4-dioxo-3,4-dihydro-2H-benzo[g]pteridin-10-yl)-acetic acid (50 mg, 0.17 mmol) and (3-amino-propyl)-phosphonic acid diethyl ester (33 mg, 0.17 mmol) in DMF (5 mL), DIPEA (0.09 mL, 0.34 mmol) and HATU (64 mg, 0.17 mmol) are added sequentially at room temperature. After 18 h the reaction mixture is diluted with water (3 mL) and purified using preparative HPLC purification (Method 1). {3-[2-(7,8-dimethyl-2,4-dioxo-3,4-dihydro-2H-benzo[g]pteridin-10-yl)-acetylamino]-propyl}-phosphonic acid diethyl ester (20 mg) is isolated following lyophilization of the appropriate fractions (Yield: 25%). LC-MS m/z 478.0 [M+H], retention time=2.48 min.

### Step 2 Preparation of {3-[2-(7,8-Dimethyl-2,4-dioxo-3,4-dihydro-2,1-benzo[g]Pteridin-10-yl)-acetylamino]-propyl}-phosphonic acid

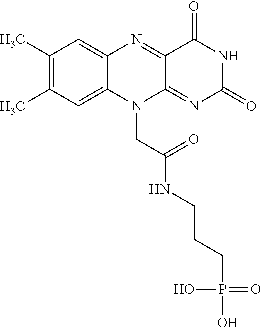

{3-[2-(7,8-Dimethyl-2,4-dioxo-3,4-dihydro-2H-benzo[g]pteridin-10-yl)-acetylamino]-propyl}-phosphonic acid diethyl ester (20 mg, 0.04 mmol) is dissolved in DCM (3 mL) and then trimethylsilylbromide is added (0.5 mL) at room temperature and the solution is stirred for 2 days. The solution is then concentrated under vacuum and purified using prep HPLC (Method 1) to obtain {3-[2-(7,8-dimethyl-2,4-dioxo-3,4-dihydro-2H-benzo[g]pteridin-10-yl)-acetylamino]-propyl}-phosphonic acid (4.2 mg) as a yellow solid (Yield: 24%). ¹H NMR (400 MHz, DMSO-d6) δ 11.41 (s, 1H), 8.29 (s, 1H), 7.94 (s, 1H), 7.58 (s, 1H), 5.29 (m, 2H), 2.47 (s, 3H), 2.40 (s, 3H), 1.60 (m, 2H), 1.47 (m, 2H), MS m/z 420.4 [M−H]—, retention time=4.34 min.

### Example 54

### 10-(2-(4-(aminomethyl)benzylamino)ethyl)-7,8-dimethylbenzo[g]pteridine-2,4(3H,10H)-dione

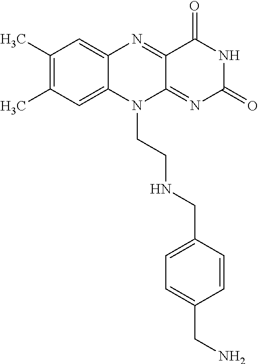

### Step 1 Preparation of 10-(2-(4-(Aminomethyl)benzylamino)ethyl)-7,8-dimethylbenzo[g]Pteridine-2,4(3H,10H)-dione

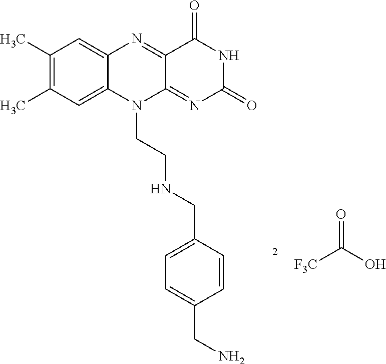

To a suspension of (7,8-dimethyl-2,4-dioxo-3,4-dihydro-2H-benzo[g]pteridin-10-yl)-acetaldehyde (Example 14) (50 mg, 0.18 mmol) in methanol (5 mL) at room temperature is added 1,4-phenylenedimethanamine (48 mg, 0.35 mmol), acetic acid (0.075 mL) and sodium cyanoborohydride (24 mg, 0.39 mmol), and the solution is stirred at room temperature for 72 h. The reaction mixture is mixed with silica gel, concentrated, and purified by column chromatography (CH₂Cl₂/MeOH/Et₃N, 90:10:1). The resulting mixture is further purified by preparative HPLC (Method 1). 10-(2-(4-(Aminomethyl)benzylamino)ethyl)-7,8-dimethylbenzo[g]pteridine-2,4(3H,10H)-dione (16 mg) is isolated following lyophilization of the appropriate fractions (Yield: 23%). ¹H NMR (400 MHz, DMSO-d6) δ 2.42 (s, 3H), 2.54 (d, 3H), 4.07 (s, 2H), 4.36 (s, 2H), 5.10 (s, 2H), 7.50 (d, 2H), 7.56 (d, 2H), 7.89 (s, 1H), 7.96 (s, 1H), 8.32 (br s, 3H) 9.29 (br s, 2H), 11.45 (s, 1H). LC-MS m/z 405.1 [M−H], retention time 4.16 min.

### Example 55

### 10-(2-(Benzylamino)ethyl)-7,8-dimethylbenzo[g]pteridine-2,4(3H,10H)-dione 2,2,2-trifluoroacetate

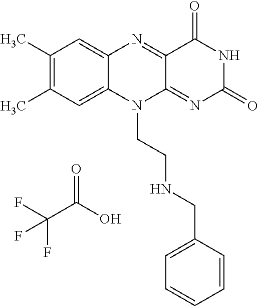

### Step 1 Preparation of 10-(2-(Benzylamino)ethyl)-7,8-dimethylbenzo[g]Pteridine-2,4(3H,10H)-dione

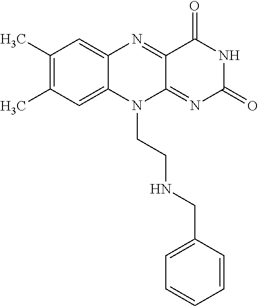

Crude material is prepared by reductive amination using a procedure similar to that of Step 2, Example 15 using 2-(7,8-dimethyl-2,4-dioxo-3,4-dihydrobenzo[g]pteridin-10(2H)-yl)acetaldehyde (Example 14) and benzylamine. This product is contaminated with 10-(2-(benzyl(methyl)amino)ethyl)-7,8-dimethylbenzo[g]pteridine-2,4(3H,10H)-dione. The next two steps are performed to isolate desired product.

### Step 2 Preparation of tert-Butyl benzyl(2-(7,8-dimethyl-2,4-dioxo-3,4-dihydrobenzo[g]pteridin-10(2H)-yl)ethyl)carbamate

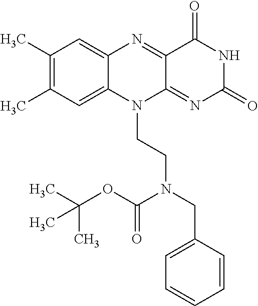

To a solution of crude 10-(2-(benzylamino)ethyl)-7,8-dimethylbenzo[g]pteridine-2,4(3H,10H)-dione (7.53 mmol) in MeOH (200 mL) is added di-tert-butyl dicarbonate (5.2 g, 23.8 mmol) and Et₃N (4 ml). The reaction was concentrated under reduced pressure and purified via silica gel chromatography (ISCO) (100% DCM to 10% MeOH/DCM) over 1 h to obtain desired product (1.85 g, 54%) as a brown solid.

### Step 3 Preparation of 10-(2-(Benzylamino)ethyl)-7,8-dimethylbenzo[g]pteridine-2,4(3H,10H)-dione 2,2,2-trifluoroacetate

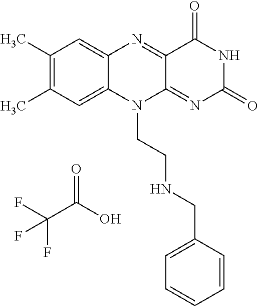

To a solution of tert-butyl benzyl(2-(7,8-dimethyl-2,4-dioxo-3,4-dihydrobenzo[g]pteridin-10(2H)-yl)ethyl)carbamate (50 mg, 0.11 mmol) in DCM (2 mL) is added TFA (2 mL) at rt. After 2 h, the reaction mixture is concentrated and the residual material is dissolved in MeOH (10 ml) and purified by preparative HPLC (Method 2). Lyophilization of combined fractions affords desired product (33.6 mg, 65%) as a brown solid. ¹H NMR (400 MHz, DMSO-d₆) δ 2.42 (s, 3H), 2.53 (s, 3H), 4.35 (s, 2H), 5.00 (m, 2H), 7.43 (m, 3H), 7.52 (m, 2H), 7.83 (s, 1H), 7.96 (s, 1H), 9.02 (s, 2H), 11.49 (s, 1H).

### Example 56

### N-[2-(7,8-Dimethyl-2,4-dioxo-3,4-dihydro-2H-benzo[g]pteridin-10-yl)-ethyl]-succinamic acid

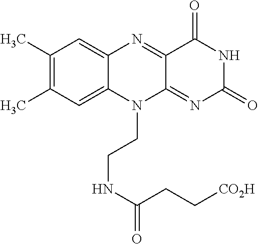

The mixture of 10-(2-aminoethyl)-7,8-dimethylbenzo[g]pteridine-2,4(3H,10H)-dione (15 mg, 0.053 mmol) (see Example 40)) and succinic anhydride (15 mg, 0.15 mmol) in pyridine (2 mL) is stirred at room temperature for 5 h. The reaction mixture is concentrated to dryness, dissolved in DMF/water (1/3) and purified by preparative HPLC (Method 4) to give, after isolation and lyophilization, N-[2-(7,8-dimethyl-2,4-dioxo-3,4-dihydro-2H-benzo[g]pteridin-10-yl)-ethyl]-succinamic acid (3.4 mg, yield: 17%). ¹H NMR (400 MHz, DMSO-d6) δ 2.18 (m, 2H), 2.34 (m, 2H), 2.40 (s, 3H), 3.47 (m, 2H), 4.46 (m, 2H), 7.87 (s, 1H), 7.91 (s, 1H), 8.11 (m, 1H), 11.35 (s, 1H), 12.10 (br.s. 1H).

### Example 57

### 7,8-Dimethyl-10-(5-(2-oxopyrimidin-1(2H)-yl)pentyl)benzo[g]pteridine-2,4(3H,10H)-dione (I) and 7,8-dimethyl-10-(5-(pyrimidin-2-yloxy)pentyl)benzo[g]pteridine-2,4(3H,10H)-dione (II)

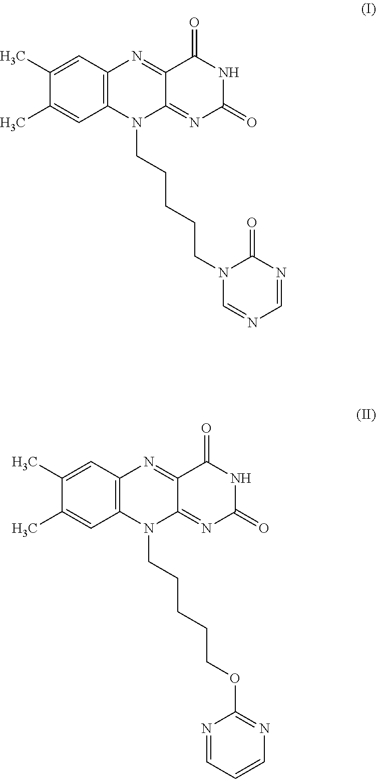

To a suspension of pyrimidin-2(1H)-one hydrochloride (100 mg, 0.755 mmol) and potassium carbonate (104 mg, 0.755 mmol) in anhydrous DMF (5 mL) is added 10-(5-bromopentyl)-7,8-dimethylbenzo[g]pteridine-2,4(3H,10H)-dione (see Intermediate 1 for preparation) (50 mg, 0.128 mmol) at room temperature. The mixture is then heated to 50° C. and stirred for 8 h, concentrated (50° C.), dissolved in DMF/water (1/3) and purified by preparative HPLC (Method 1) to give, after lyophilization, 7,8-dimethyl-10-[5-(2-oxo-2H-pyrimidin-1-yl)-pentyl]-10H-benzo[g]pteridine-2,4-dione (I) (10.9 mg, yield: 21%). ¹H NMR (400 MHz, DMSO-d6) δ 1.45 (m, 2H), 1.76 (m, 4H), 2.39 (s, 3H), 2.51 (s, 3H), 3.95 (m, 2H), 4.57 (m, 2H), 6.58 (dd, 1H), 7.83 (s, 1H), 7.90 (s, 1H), 8.50 (dd, 1H), 8.61 (m, 1H), 11.31 (s, 1H); LC-MS m/z 407.1 [M+H]⁺ and 7,8-Dimethyl-10-[5-(pyrimidin-2-yloxy)-pentyl]-10H-benzo[g]pteridine-2,4-dione (II) (4 mg, yield: 7.7%). ¹H NMR (400 MHz, DMSO-d6) δ 1.59 (m, 2H), 1.81 (m, 4H), 2.40 (s, 3H), 4.32 (m, 2H), 4.61 (m, 2H), 7.12 (m, 1H), 7.83 (s, 1H), 7.91 (s, 1H), 8.58 (d, 2H), 11.30 (s, 1H).

### Example 58

### 1-(3-(2-(7,8-Dimethyl-2,4-dioxo-3,4-dihydrobenzo[g]pteridin-10(2H)-yl)ethylamino)propyl)guanidine

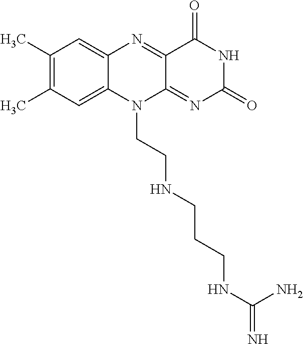

### Step 1 Preparation of 10-(2-(3-Aminopropylamino)ethyl)-7,8-dimethylbenzo[g]pteridine-2,4(3H,10H)-dione

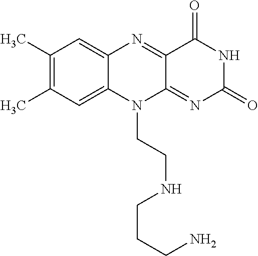

Prepared by reductive amination using a procedure similar to that of Example 15, step 2, using 2-(7,8-dimethyl-2,4-dioxo-3,4-dihydrobenzo[g]pteridin-10(2H)-yl)acetaldehyde (Example 14)) and propane-1,3-diamine (15 equivalents) as starting materials.

### Step 2 Preparation of 1-(3-(2-(7,8-Dimethyl-2,4-dioxo-3,4-dihydrobenzo[2]Pteridin-10(2H)-yl)ethylamino)propyl)guanidine

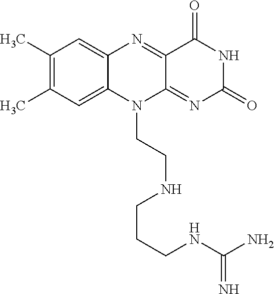

A mixture of 10-(2-(3-aminopropylamino)ethyl)-7,8-dimethylbenzo[g]pteridine-2,4(3H,10H)-dione (66 mg, 0.19 mmol), 3,5-dimethyl-1H-pyrazole-1-carboximidamide (33 mg, 0.16) and DIPEA (45 mg, 0.348 mmol) in DMF (3 ml) are stirred at rt for 16 h. The reaction is concentrated under reduced pressure, dissolved in ACN (5 ml)/water (2 ml) and purified by preparative HPLC (Method 2). Lyophilization of combined fractions affords desired product (30.5 mg, 41%). ¹H NMR (400 MHz, CD₃OD) δ 2.03 (m, 2H), 2.50 (s, 3H), 2.62 (s, 3H), 3.37 (m, 4H), 3.67 (m, 2H), 5.11 (m, 2H), 7.83 (s, 1H), 8.00 (s, 1H).

### Example 59

### 4-((2-(7,8-Dimethyl-2,4-dioxo-3,4-dihydrobenzo[g]pteridin-10(2H)-yl)ethylamino)methyl)benzoic acid

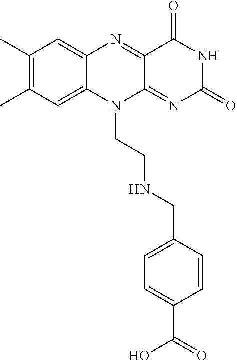

To a solution of (7,8-dimethyl-2,4-dioxo-3,4-dihydro-2H-benzo[g]pteridin-10-yl)-acetaldehyde (Example 14)) (50 mg, 0.176 mmol) in methanol (5 mL) are added 4-(aminomethyl)benzoic acid (26.6 mg, 0.176 mmol) and acetic acid (75 μL) at rt. The reaction is stirred at 50° C. for 30 min. The reaction is cooled to room temperature and sodium cyanoborohydride (25 mg, 0.39 mmol) is added and the reaction mixture is stirred at 50° C. for 24 h. The product precipitates from the reaction mixture and is filtered and washed with MeOH (10 mL) and dried overnight under vacuum (50.0 mg, 69% yield). ¹H NMR (400 MHz, DMSO) δ 2.38 (s, 3H), 2.46 (s, 3H), 2.91 (m, 2H), 3.84 (s, 2H), 4.70 (m, 2H), 7.39 (d, 2H), 7.82 (m, 4H), 11.30 (s, 1H). ESI(−) m/z=418.5

### Example 60

### 2-Amino-5-(2-(7,8-dimethyl-2,4-dioxo-3,4-dihydrobenzo[g]pteridin-10(2H)-yl)ethylamino)benzoic acid

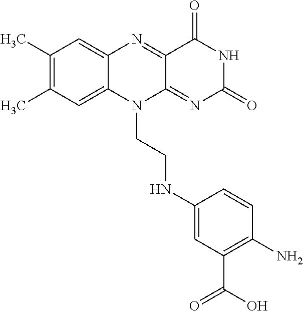

2-Amino-5-(2-(7,8-dimethyl-2,4-dioxo-3,4-dihydrobenzo[g]pteridin-10(2H)-yl)ethylamino)benzoic acid is synthesized from (7,8-dimethyl-2,4-dioxo-3,4-dihydro-2H-benzo[g]pteridin-10-yl)-acetaldehyde (Example 14) (50 mg, 0.176 mmol) and 2,5-diaminobenzoic acid dihydrochloride (44 mg, 0.195 mmol) following the procedure described for Example 59. The solution is concentrated under reduced pressure, and the residue dissolved in DMSO (1 mL), filtered, and purified by preparatory HPLC (Method 2). The desired product (12.7 mg) is isolated after lyophilization of the fractions (yield: 18.6%). ¹H NMR (400 MHz, DMSO) δ 2.30 (s, 3H), 2.38 (s, 3H), 2.56 (m, 2H), 4.72 (m, 2H), 6.18 (m, 1H), 6.73 (m, 2H), 7.44 (m, 2H), 7.53 (s, 1H), 7.89 (s, 1H), 11.38 (s, 1H) ESI(−) m/z=419.1.

### Example 61

### 7-{7-methyl-2,4-dioxo-8-[(3R)-pyrrolidin-3-ylamino]-3,4-dihydrobenzo[g]pteridin-10(2H)-yl}heptanoic acid trifluoroacetate salt

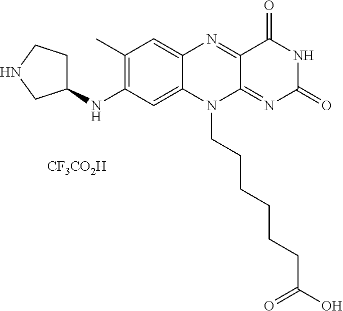

### Step 1 Preparation of tert-Butyl (3R)-3-{[10-(7-ethoxy-7-oxoheptyl)-7-methyl-2,4-dioxo-2,3,4,10-tetrahydrobenzo[g]pteridin-8-yl]amino}pyrrolidine-1-carboxylate

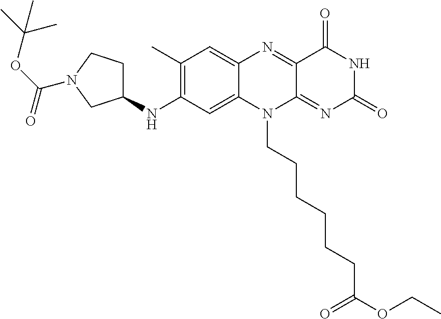

A sealed 20 mL scintillation vial containing a solution of ethyl 7-(8-chloro-7-methyl-2,4-dioxo-3,4-dihydrobenzo[g]pteridin-10(2H)-yl)heptanoate (103 mg, 0.24 mmol), tert-butyl (3R)-3-aminopyrrolidine-1-carboxylate (229 mg, 1.23 mmol) and NMP (5.0 mL) is stirred at 90° C. for 24 h. The reaction is cooled to rt and partitioned between saturated sodium bicarbonate and DCM (50 mL each). The layers are separated and the aqueous extracted with DCM (3×25 mL). The organics are combined, dried with anhydrous sodium sulfate and concentrated. The residue is subjected to preparative reverse phase chromatography to give 34 mg (24%) of desired product as an amorphous red solid after lyophilization. ¹H NMR (400 MHz, DMSO-d₆) δ 11.0 (1H, s), 7.70 (1H, s), 6.79 (1H, m), 6.58 (1H, s), 4.01 (2H, q), 2.30 (3H, s), 1.41 & 1.39 (9H, s), 1.16 (3H, t). MS (ESI+) for C₂₉H₄₀N₆O₆ m/z 569.1 (M+H)⁺, retention time: 3.63 min (System C).

### Step 2 Preparation of 7-[-{[(3R)-1-I-Butoxycarbonyl)pyrrolidin-3-yl]amino}-7-methyl-2,4-dioxo-3,4-dihydrobenzo[g]pteridin-10(2H)-yl]heptanoic acid

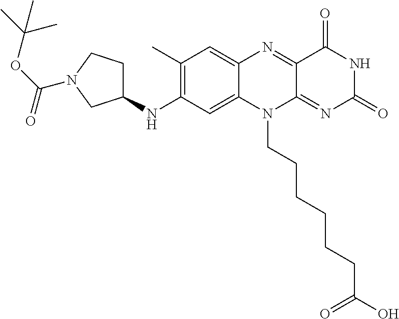

A solution of tert-butyl (3R)-3-{[10-(7-ethoxy-7-oxoheptyl)-7-methyl-2,4-dioxo-2,3,4,10-tetrahydrobenzo[g]pteridin-8-yl]amino}pyrrolidine-1-carboxylate (89.0 mg, 0.156 mmol) and 1.0 M lithium hydroxide (0.5 mL, 0.5 mmol) in THF (2.0 mL,) and water (0.5 mL) is stirred at rt for 2 h. Acetic acid (0.10 mL, 1.8 mmol) is added and the reaction mixture is concentrated. The red residue is suspended in water (5 mL), filtered, washed with water and dried in vacuo to give 52 mg (61%) of the desired product as a red solid. ¹H NMR (400 MHz, DMSO-d₆) δ 11.0 (1H, s), 7.69 (1H, s), 6.78 (m, 1H), 6.58 (1H, s), 2.29 (3H, s), 1.41 and 1.39 (9H, s). MS (ESI+) for C₂₇H₃₆N₆O₆ m/z 541.2 (M+H)+, retention time: 3.07 min (System C).

### Step 3 Preparation of 7-{7-Methyl-2,4-dioxo-8-[(3R)-pyrrolidin-3-ylamino]-3,4-dihydrobenzo[g]pteridin-10(2H)-yl}heptanoic acid trifluoroacetate salt

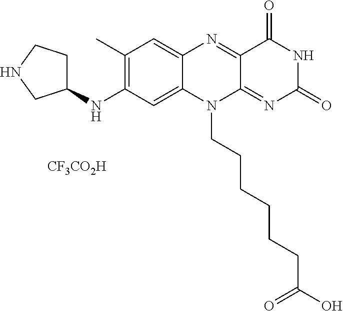

A slurry of 7-[8-{[(3R)-1-(tert-butoxycarbonyl)pyrrolidin-3-yl]amino}-7-methyl-2,4-dioxo-3,4-dihydrobenzo[g]pteridin-10(2H)-yl]heptanoic acid (35.0 mg, 0.065 mmol) in TFA (0.9 mL) and water (6.0 mL) is stirred at rt for 18 h. This solution is then lyophilized to give 34 mg (94%) of desired product as a red solid. ¹H NMR (400 MHz, D₂O) δ 7.30 (1H, s), 6.11 (1H, s), 4.47 (1H, m), 4.38 (1H, m), 4.29 (1H, m), 3.68 (1H, m), 3.55-3.46 (3H, m), 2.45 (1H, M), 2.28 (3H, m), 2.14 (3H, s), 1.63 (2H, m), 1.51 (2H, m) 1.38-1.30 (4H, m). MS (ESI+) for C₂₂H₂₈N₆O₄ m/z 441.2 (M+H)+, retention time: 1.99 min (System C).

### Example 62

### 7-{7-Methyl-2,4-dioxo-8-[(3S)-pyrrolidin-3-ylamino]-3,4-dihydrobenzo[g]pteridin-10(2H)-yl}heptanoic acid trifluoroacetate salt

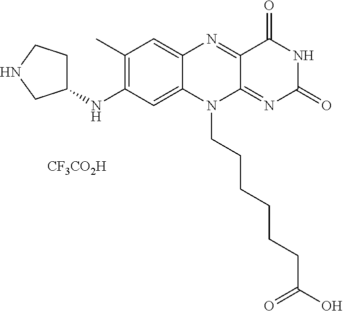

### Step 1 Preparation of tert-Butyl (3S)-3-{[10-(7-ethoxy-7-oxoheptyl)-7-methyl-2,4-dioxo-2,3,4,10-tetrahydrobenzo[g]pteridin-8-yl]amino}pyrrolidine-1-carboxylate

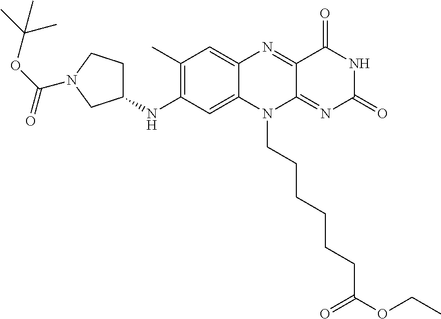

A sealed 20 mL scintillation vial containing a solution of ethyl 7-(8-chloro-7-methyl-2,4-dioxo-3,4-dihydrobenzo[g]pteridin-10(2H)-yl)heptanoate (50.0 mg, 0.12 mmol) and tert-butyl (3S)-3-aminopyrrolidine-1-carboxylate (100 mg, 0.6 mmol) in NMP (3.0 mL) is stirred at 90° C. for 48 h. The reaction is concentrated and subjected to preparative reverse phase chromatography to provide 20 mg (30%) of the desired product as a red solid after lyophilization. ¹H NMR (400 MHz, DMSO-d₆) δ 11.0 (1H, s), 7.70 (1H, s), 6.79 (1H, m), 6.58 (1H, s), 4.01 (2H, q), 2.30 (3H, s), 1.39 (9H, s), 1.16 (3H, t). MS (ESI+) for C₂₉H₄₀N₆O₆ m/z 569.1 (M+H)+, retention time: 3.65 min (System C).

### Step 2 Preparation of 7-[8-{[(3S)-1-I-Butoxycarbonyl)pyrrolidin-3-yl]amino}-7-methyl-2,4-dioxo-3,4-dihydrobenzo[g]pteridin-10(2H)-yl]heptanoic acid

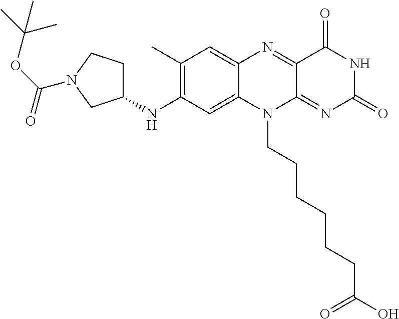

To a well-stirred slurry of tert-butyl (35)-3-{[10-(7-ethoxy-7-oxoheptyl)-7-methyl-2,4-dioxo-2,3,4,10-tetrahydrobenzo[g]pteridin-8-yl]amino}pyrrolidine-1-carboxylate (20.0 mg, 0.04 mmol) and water (1.0 mL) in THF (3.0 mL,) at rt is added 1.0 M lithium hydroxide (0.50 mL). The reaction is stirred at rt for 1 h and quenched with acetic acid (50 uL). This mixture is concentrated and re-suspended in water (5 mL). The precipitate is collected and dried in vacuo to give 11 mg (58%) of desired product as a red solid. ¹H NMR (400 MHz, DMSO-d₆) δ 11.9 (1H, br s), 11.0 (1H, s), 7.69 (1H, s), 6.79 (m, 1H), 6.58 (1H, s), 2.29 (3H, s), 1.41 & 1.39 (9H, s). MS (ESI+) for C₂₇H₃₆N₆O₆ m/z 541.0 (M+H)+, retention time: 3.07 min (System C).

### Step 3 Preparation of 7-{7-Methyl-2,4-dioxo-8-[(3S)-pyrrolidin-3-ylamino]-3,4-dihydrobenzo[g]pteridin-10(2H)-yl}heptanoic acid trifluoroacetate salt

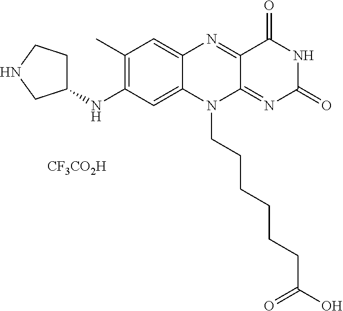

A slurry of 7-[8-{[(3S)-1-(tert-butoxycarbonyl)pyrrolidin-3-yl]amino}-7-methyl-2,4-dioxo-3,4-dihydrobenzo[g]pteridin-10(2H)-yl]heptanoic acid (34.5 mg, 0.064 mmol) in TFA (0.3 mL) and water (2.0 mL) is stirred at rt for 18 h. The solution is lyophilized to give 33 mg of desired product as a red solid (93%). ¹H NMR (400 MHz, D₂O) δ 7.30 (1H, s), 6.11 (1H, s), 4.47 (1H, m), 4.38 (1H, m), 4.29 (1H, m), 3.68 (1H, m), 3.55-3.46 (3H, m), 2.45 (1H, M), 2.28 (3H, m), 2.14 (3H, s), 1.63 (2H, m), 1.51 (2H, m), 1.38-1.30 (4H, m). MS (ESI+) for C₂₂H₂₈N₆O₄ m/z 441.2 (M+H)+, retention time: 1.99 min (System C).

### Example 63

Sodium 7-[7-methyl-2,4-dioxo-8-(trifluoromethyl)-3,4-dihydrobenzo[g]pteridin-10(2H)-yl]heptanoate

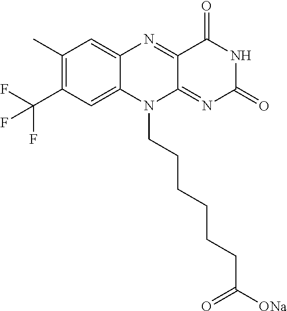

### Step 1 Preparation of Ethyl 7-{[4-methyl-3-(trifluoromethyl)phenyl]amino}-heptanoate

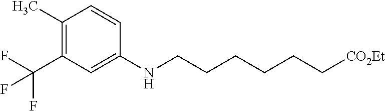

An N₂ flushed solution of ethyl 7-bromoheptanoate (1.3 mL, 6.7 mmol), 4-methyl-3-(trifluoromethyl)aniline (1.3 mL, 9.0 mmol), and DIPEA (2.0 mL, 11 mmol) is shaken at 70° C. After 18 h, the mixture is removed from heat, diluted with heptane (10 ml) and is allowed to stand at room temperature for 5 days. The mixture is then filtered (5×10 mL heptane rinses). The heptane layer is washed with water (50 mL), stripped to dryness, and then chromatographed on silica gel using a gradient from heptane to 20% CH₂Cl₂/heptane. This provides desired product as a colorless oil (1.66 g, 75% yield). MS (ESI+) for C₁₇H₂₄F₃NO₂ m/z 332.15 (M+H)+, retention time 6.44 min (System B).

### Step 2 Preparation of Ethyl 7-{[4-methyl-2-nitro-5-(trifluoromethyl)phenyl]amino}heptanoate

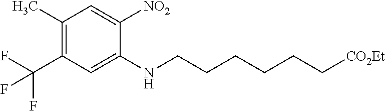

A well-stirred mixture of ethyl 7-{[4-methyl-3-(trifluoromethyl)phenyl]amino}-heptanoate (0.333 g, 1.00 mmol) and acetic acid (4 mL, 70 mmol) is cooled to 10-12° C. (internally monitored). To this mixture is added nitric acid (0.0936 mL, 2.01 mmol) dropwise followed by acetic anhydride (1.0 mL, 1.0E1 mmol) and the cooling bath is removed. After 72 h, the mixture is mixed with ice (5 mL), diluted with water (10 mL), saturated aqueous NaHCO₃(10 mL), and heptanes (30 mL). More saturated aqueous NaHCO₃ is added until the mixture is basic, then the organic layer is isolated, washed with water and concentrated in vacuo to a reddish oil. The residue is chromatographed on silica gel using 2% EtOAc/heptane to give the desired product as a red oil (0.080 g, 21% yield). ¹H NMR (400 MHz, CDCl₃) δ 1.26 (3H, t), 1.36-1.53 (4H, m), 1.60-1.81 (4H, m), 2.32 (2H, t), 2.38 (3H, br s), 3.27-3.35 (2H, m), 4.14 (2H, q), 7.12 (1H, s), 7.90 (1H, br s), 8.08 (1H, s). MS (ESI+) for C₁₇H₂₃F₃N₂O₄ m/z 377.0 (M+H)⁺, retention time 7.87 min (System B).

### Step 3 Preparation of Ethyl 7-{[2-amino-4-methyl-5-(trifluoromethyl)phenyl]amino}heptanoate

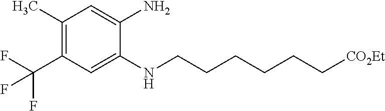

An N₂ flushed mixture of ethyl 7-{[4-methyl-2-nitro-5-(trifluoromethyl)phenyl]amino}-heptanoate (73 mg, 0.19 mmol) and Raney nickel (0.03 mL in H₂O) in EtOH (2 mL) is stirred under an H₂ balloon. After overnight stirring, additional Raney nickel (0.03 mL in H₂O) is added and stirring under H₂ is continued. After 72 h, the mixture is filtered through a 1 cm silica gel column (1 mL EtOH rinse), fresh Raney nickel (0.03 mL in H₂O) is added and stirring under H₂ is resumed. After 2 hr, the reaction mixture is filtered and stripped to provide the desired product as an oil (65 mg, 83% yield). MS (ESI+) for C₁₇H₂₅F₃N₂O₂ m/z 347.1 (M+H)⁺, retention time 5.65 min (System B).

### Step 4 Preparation of Ethyl 7-[7-methyl-2,4-dioxo-8-(trifluoromethyl)-3,4-dihydrobenzo[g]pteridin-10(2H)-yl]heptanoate

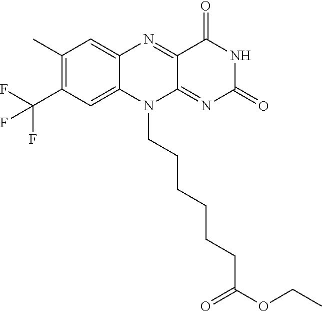

An N₂ flushed mixture of ethyl 7-{[2-amino-4-methyl-5-(trifluoromethyl)phenyl]-amino}heptanoate (62 mg, 0.12 mmol), alloxan monohydrate (0.015 g, 0.094 mmol) and boric acid (0.03 g, 0.5 mmol) in acetic acid (1.5 mL) is stirred at rt. After 18 h, additional boric acid (0.03 g, 0.5 mmol) is added and stirring is continued. After another 18 h, additional alloxan monohydrate (0.020 g, 0.12 mmol) is added and the mixture is heated to 50° C. for 24 h. The mixture is concentrated and chromatographed on silica gel using a gradient from 20 to 50% EtOAc/DCM. This provided 70 mg of a semi-purified solid that is subjected to preparative silica gel chromatography (divided onto two 0.1 mm prep silica gel plates and elution with 5% Et₀H/DCM). This provides 14 mg (17%) of the desired product as a yellow solid. ¹H NMR (400 MHz, DMSO-d₆) δ 1.16 (3H, t), 1.29-1.48 (4H, m), 1.49-1.58 (2H, m), 1.63-1.76 (2H, m), 2.18-2.36 (2H, m), 2.59(3H, s), 3.94-4.10 (2H, m), 4.62 (2H, br s), 8.06 (1H, s), 8.22 (1H, s), 11.49 (1H, br s). MS (ESI−) for C₂₁H₂₃F₃N₄O₄ m/z 451.1 (M−H)⁻, retention time: 5.83 min (System B).

### Step 5 Preparation of Sodium 7-[7-methyl-2,4-dioxo-8-(trifluoromethyl)-3,4-dihydrobenzo[g]pteridin-10(2H)-yl]heptanoate

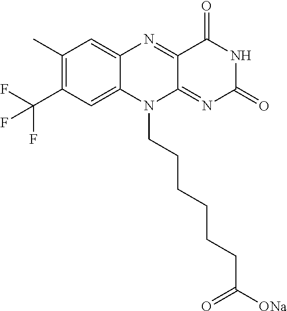

To a solution of ethyl 7-[7-methyl-2,4-dioxo-8-(trifluoromethyl)-3,4-dihydrobenzo[g]pteridin-10(2H)-yl]heptanoate (13.5 mg, 0.0239 mmol) in THF (0.5 mL) is added 12 M aqueous HCl (0.010 mL, 0.12 mmol). After 4 days, additional 12 M aqueous HCl (0.002 mL, 0.02 mmol) is added. After 5 days, the mixture is cooled in ice, diluted with ½ mL of ice and brought to pH 8-9 with aqueous NaHCO₃ at which point two layers formed. The layers are separated and the aqueous layer is washed with EtOAc (4×1 mL) and purified on reverse phase silica (C18, elution with water then methanol). Concentration provides the desired product as an orange solid (6 mg, 47%). ¹H NMR (400 MHz, DMSO-d₆) δ 1.18-1.47 (7 H, m), 1.65 (2H, br s), 1.76-1.88 (2H, m), 2.55 (3H, br s), 4.51 (2H, br s), 7.92 (1H, br s), 8.10 (1H, br s). MS (ESI−) for C₁₉H₁₈F₃N₄NaO₄ m/z 423.0 (M−H)⁻, retention time 4.92 min (System B).

### Example 64

### Potassium 7-(7,8-dimethyl-2,4-dioxo-3,4-dihydrobenzo[g]pteridin-10(2H)-yl)-5-hydroxyheptanoate

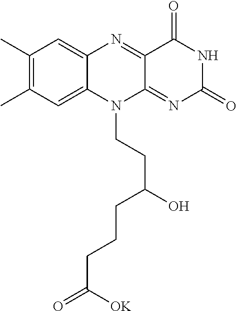

### Step 1 Preparation of 6-{2-[(4,5-Dimethyl-2-nitrophenyl)amino]ethyl}tetrahydro-2H-pyran-2-one

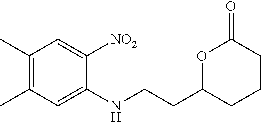

A mixture of 4,5-dimethyl-2-nitroaniline (0.16 g, 0.97 mmol), 6-(2-bromoethyl)-tetrahydro-2H-pyran-2-one (0.167 g, 0.806 mmol) [Molander, G. A.; McKie, J. A. J. Org. Chem. 1993 58, 7216], and DIPEA (3 mL, 20 mmol) in a capped vial is shaken at 120° C. After 6 h 40 minutes, the reaction mixture is cooled, concentrated and chromatographed on silica gel (DCM followed by 0.3% MeOH/DCM) giving desired product as an orange/red solid (0.110 g, 46% yield). MS (ESI+) for C₁₅H₂₀N₂O₄ m/z 293.3 (M+H)⁺, retention time 5.99 min (System B).

### Step 2 Preparation of 6-{2-[(2-Amino-4,5-dimethylphenyl)amino]ethyl}tetrahydro-2H-pyran-2-one

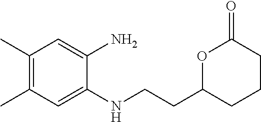

An N₂ flushed mixture of 6-{2-[(4,5-dimethyl-2-nitrophenyl)amino]ethyl}tetrahydro-2H-pyran-2-one (0.108 g, 0.369 mmol) and Raney nickel (0.03 mL in H₂O) in EtOH (30 mL) is stirred under H₂ (balloon). After 18 h, additional Raney nickel (0.03 mL in H₂O) is added and stirring under H₂ is continued. After 3 ch, the reaction mixture is filtered and concentrated to an oil (95 mg, 98%). MS (ESI+) for C₁₅H₂₂N₂O₂ m/z 263.16 (M+H)⁺, retention time 4.11 min (System B).

### Step 3 Preparation of 7,8-Dimethyl-10-[2-(6-oxotetrahydro-2H-pyran-2-yl)ethyl]benzo[g]pteridine-2,4(3H,10H)-dione

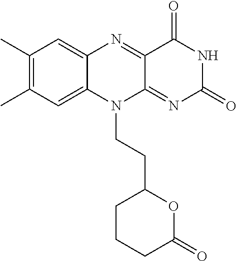

An N₂ flushed mixture of 6-{2-[(2-amino-4,5-dimethylphenyl)amino]ethyl}tetrahydro-2H-pyran-2-one (0.120 g, 0.274 mmol), alloxan monohydrate (0.066 g, 0.41 mmol) and boric acid (0.1 g, 2 mmol) in acetic acid (3 mL) is stirred at room temperature. After 18 h, additional alloxan monohydrate (0.0330 g, 0.206 mmol) is added. After 2 days, the reaction mixture is concentrated to a dark red solid. The solid is suspended in water (5 mL) and extracted with DCM (3×3 mL). The combined organic layers are stripped to dryness, mixed with THF (5 mL) and filtered (2×1 mL THF washes). The filtered solid is washed with MeOH (3×2 mL) and dried under high vacuum overnight to give a yellow-orange solid (11 mg, 31% yield). ¹H NMR (400 MHz, DMSO-d₆) δ 1.50-1.64 (1H, m), 1.92-2.10 (3H, m), 2.40 (3H, s), 4.46-4.84 (3H, m), 7.78 (1 H, s), 7.92 (1H, s), 11.32 (1H, br s). MS (ESI−) for C₁₉H₂₀N₄O₄ m/z 367.0 (M−H)⁻, retention time: 4.14 min (System B).

### Step 4 Preparation of Potassium 7-(7,8-dimethyl-2,4-dioxo-3,4-dihydrobenzo[g]pteridin-10(2H)-yl)-5-hydroxyheptanoate

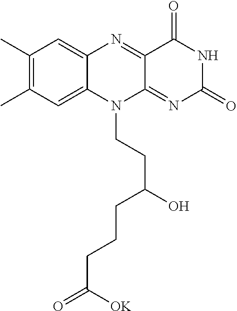

A mixture of 7,8-dimethyl-10-[2-(6-oxotetrahydro-2H-pyran-2-yl)ethyl]benzo[g]pteridine-2,4(3H,10H)-dione (6 mg, 0.01 mmol) and potassium hydroxide (1.2 mg, 0.018 mmol) in water (1.2 mL) is stirred at room temperature for 1 h. The orange solution is transferred (in water) to a 0.5 g C18 reverse phase silica gel column and eluted with water (5 mL fractions being collected). The product containing fractions are combined and concentrated to give desired product as an orange solid (6.0 mg, 86%). ¹H NMR (400 MHz, D₂O) δ 1.38-2.03 (7 H, m), 2.34-2.37 (8 H, m), 2.48 (3H, s), 3.76 (1H, br s), 7.61 (1H, br s), 7.66 (1H, s). MS (ESI+) for C₁₉H₂₁KN₄O₅ m/z 424.88 (M)⁺, retention time: 3.87 min (System B).

### Example 65

### 7-(8-Cyclopropyl-7-methyl-2,4-dioxo-3,4-dihydrobenzo[g]pteridin-10(2H)-yl)]heptanoic acid

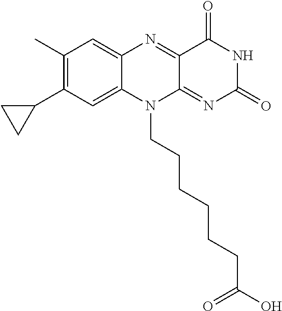

### Step 1 Preparation of Ethyl 7-[(5-cyclopropyl-4-methyl-2-nitrophenyl)amino]-heptanoate

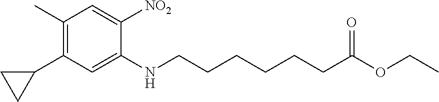

A mixture of ethyl 7-[(5-chloro-4-methyl-2-nitrophenyl)amino]heptanoate (218 mg, 0.636 mmol), cyclopropylboronic acid) (81.9 mg, 0.954 mmol), [1,1′-bis(diphenylphosphino)ferrocene]dichloropalladium(II), complex with dichloromethane (1:1) (31 mg, 0.038 mmol), and cesium carbonate (622 mg, 1.91 mmol) in N,N-dimethylacetamide (4.38 mL), rapidly stirred under nitrogen, is placed into a preheated 150° C. bath for 15 minutes. The reaction is allowed to cool to room temperature, stir open to air for 24 hours, and is then partitioned between aqueous sodium bicarbonate and DCM. The organic layer is washed with aqueous sodium bicarbonate then brine and is dried with anhydrous sodium sulfate. After concentration, the residue is subjected to flash chromatography on a 40×95 mm silica gel column (Silicycle, 230-400 mesh, elution with 75% DCM/hexanes) to give 85.2 mg of desired product as orange oil. MS (ESI+) for C₁₉H₂₈N₂O₄ m/z 349.2 (M+H)+, retention time: 5.62 min (System D).

### Step 2 Preparation of Ethyl 7-[(2-amino-5-cyclopropyl-4-methylphenyl)amino]-heptanoate

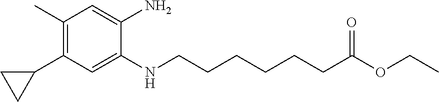

A well-stirred mixture of Raney nickel (50 mg, 0.85 mmol) and ethyl 7-[(5-cyclopropyl-4-methyl-2-nitrophenyl)amino]heptanoate (99 mg, 0.28 mmol) in ethanol (6 mL) is alternately evacuated then covered with 1 atmosphere of hydrogen (3×) (balloon). After an hour at room temperature, the mixture is filtered through Celite® and concentrated to give 90 mg (98%) as an oil. MS (ESI+) for C₁₉H₃₀N₂O₂ m/z 319.3 (M+H)+, retention time: 3.60 min (System D).

### Step 3 Preparation of Ethyl 7-(8cyclopropyl-7-methyl-2,4-dioxo-3,4-dihydrobenzo[g]pteridin-10(2H)-yl]heptanoate

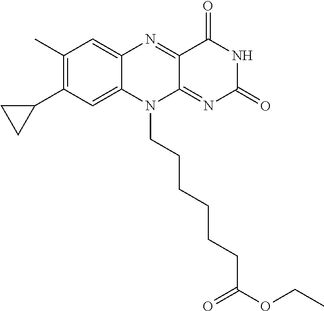

Ethyl 7-[(2-amino-5-cyclopropyl-4-methylphenyl)amino]heptan-oate (90 mg, 0.28 mmol) is dissolved in acetic acid (5 mL) and concentrated to dryness. The residue is dissolved in acetic acid (3 mL), and boric acid (87.8 mg, 1.42 mmol) is added followed by alloxan monohydrate (50.0 mg, 0.312 mmol). The flask is wrapped in aluminum foil and the solution is stirred at room temperature under nitrogen for 3 hours. The reaction is concentrated in vacuo and the residue is flash chromatographed on a C18 reverse phase silica gel column eluted with acetonitrile/water. This provided a mixture that is concentrated and flash chromatographed on a 28×75 mm silica gel column (Silicycle, 230-400 mesh, elution with 50% and 80% ethyl acetate/DCM) to give 68 mg (56%) of product as yellow solid. ¹H NMR (400 MHz, CDCl₃) δ 0.86 (m, 2H), 1.28 (m, 5 H), 1.46 (m, 2H), 1.56 (m, 2H), 1.69 (m, 2H), 1.88 (m, 2H), 2.18 (m, 1H), 2.35 (t, 2H), 2.62 (s, 3H), 4.15 (m, 2H), 4.71 (br s, 2H), 7.15 (s, 1H), 8.09 (s, 1H), 8.43 (s, 1H). MS (ESI+) for C₂₄H₂₉N₄O₄ m/z 425.2 (M+H)+, retention time: 3.70 min (System D).

### Step 4 Preparation of 7-(8-Cyclopropyl-7-methyl-2,4-dioxo-3,4-dihydrobenzo[g]pteridin-10(2H)-yl)]heptanoic acid

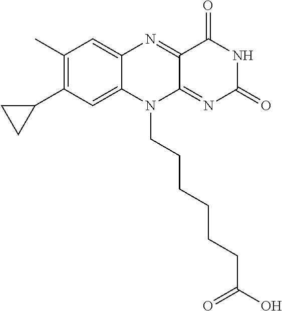

7-(8-Cyclopropyl-7-methyl-2,4-dioxo-3,4-dihydrobenzo[g]pteridin-10(2H)-yl)]heptanoate (4) (45.0 mg, 0.106 mmol) is suspended in THF (3.0 mL) with rapid stirring. Lithium hydroxide (aqueous 1.0M, 1.06 mL) is added at room temperature, and after 1 hour the reaction is quenched with acetic acid (0.060 mL, 1.06 mmol). The mixture is concentrated in vacuo and re-suspended in water. The solid is collected by filtration, washed with water, and dried under vacuum to give 34.0 mg of desired product as yellow solid. ¹H NMR (400 MHz, DMSO-d₆) δ 0.94 (m, 2H), 1.15 (m, 2H), 1.37 (m, 2H), 1.44 (m, 2H), 1.52 (m, 2H), 1.68 (m, 2H), 2.21 (m, 3H), 2.55 (s, 3H), 4.62 (m, 2 H), 7.23 (s, 1H), 7.92 (s, 1H), 11.3 (s, 1H), 12.0 (s, 1H). MS (ESI+) for C₂₁H₂₄N₄O₄ m/z 397.1 (M+H)⁺, retention time: 3.10 min (System D).

### Example 66

### N-(Benzyloxy)-6-(7,8-dimethyl-2,4-dioxo-3,4-dihydrobenzo[g]pteridin-10(2H)-yl)hexanamide

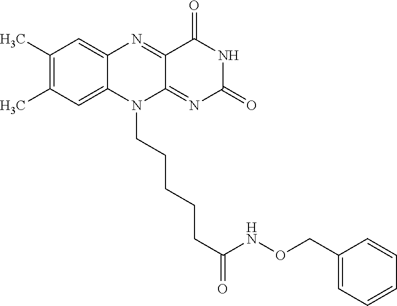

To a stirred suspension of 7-(7,8-dimethyl-2,4-dioxo-3,4-dihydrobenzo[g]pteridin-10(2H)-yl)hexanoic acid (80 mg, 0.16 mmol) (prepared using the synthesis of steps 1-3 of Example 25) in DCM (1 mL), at 0° C. under argon, thionyl chloride (2 mL) is added. The reaction mixture is stirred for 10 min, warmed to room temperature, and stirred for 30 min at room temperature. The reaction mixture is concentrated under vacuo. The residue is dissolved in DCM (5 mL) and TEA (0.5 mL). DMAP (5 mg) and O-benzylhydroxylamine hydrochloride (107 mg, 0.67 mmol) is added under argon. The reaction mixture is stirred at rt overnight. The reaction mixture is concentrated under vacuo and purified by silica gel column chromatography using 0 to 10% MeOH in DCM as eluent. N-(benzyloxy)-6-(7,8-dimethyl-2,4-dioxo-3,4-dihydrobenzo[g]pteridin-10(2H)-yl)hexanamide (71 mg, 69%) is obtained as a yellow solid after concentrating the desired fractions under vacuo. ¹H NMR (400 MHz, CDCl₃) δ 1.66 (m, 2H), 1.90 (m, 4H), 2.34 (m, 2H), 2.49 (s, 3H), 2.61 (s, 3H), 4.60 (m, 2H), 5.0 (s, 2H), 5.42 (s, 1H), 7.34 (m, 2H), 7.45 (m, 3H), 8.11 (s, 1H), 8.51 (s, 1H), 10.00 (s, 1H). LC-MS m/z 462.1 [M+H]⁺, retention time 4.33 min.

### Example 67

### 6-(7,8-Dimethyl-2,4-dioxo-3,4-dihydrobenzo[g]pteridin-10(2H)-yl)-N-hydroxyhexanamide

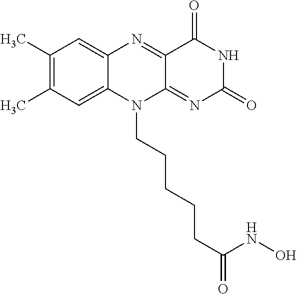

N-(benzyloxy)-6-(7,8-dimethyl-2,4-dioxo-3,4-dihydrobenzo[g]pteridin-10(2H)-yl) hexanamide (52 mg, 0.11 mmol) (Example above) is dissolved in methanol (5 mL), purged with argon for 10 min. Palladium on carbon (10%, 20 mg) is added, followed by hydrogen (via balloon). The reaction mixture is stirred under hydrogen at rt overnight. The reaction mixture is filtered through celite and the celite is washed with methanol (10 mL). The filtrate is concentrated under vacuo to obtain crude 6-(7,8-dimethyl-2,4-dioxo-3,4-dihydrobenzo[g]pteridin-10(2H)-yl)-N-hydroxyhexanamide (48 mg). The residue is purified by preparative TLC using 10% methanol in DCM as eluent to afford desired 6-(7,8-dimethyl-2,4-dioxo-3,4-dihydrobenzo[g]pteridin-10(2H)-yl)-N-hydroxyhexanamide (12.0 mg, 30%). ¹H NMR (400 MHz, MeOH-d₄) δ 1.63 (m, 2H), 1.77 (m, 2H), 1.92 (m, 2H), 2.21 (t, 2H), 2.49 (s, 3H), 2.61 (s, 3H), 4.73 (t, 2H), 7.76 (s, 1H), 7.94 (s, 1H). LC-MS m/z 372.0 [M+H]⁺, retention time 3.56 min.

### Example 68

### 7-(7,8-Dimethyl-2,4-dioxo-3,4-dihydrobenzo[g]pteridin-10(2H)-yl)-N-(methyl sulfonyl)hexanamide

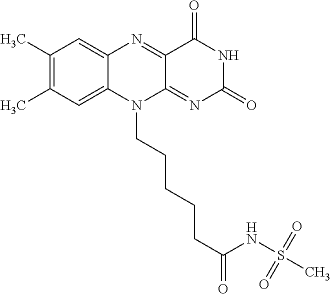

To a stirred suspension of 7-(7,8-dimethyl-2,4-dioxo-3,4-dihydrobenzo[g]pteridin-10(2H)-yl)hexanoic acid (prepared using the synthesis of steps 1-3 of Example 25) (57 mg, 0.16 mmol) in DCM (1 mL), at 0° C. under argon, thionyl chloride (2 mL) is added. The reaction mixture is stirred, warmed to room temperature, and stirred for 30 min at room temperature. The reaction mixture is concentrated under vacuo. In the meantime, to a stirred solution of methanesulfonamide (23 mg, 0.24 mmol) in anhydrous THF (5 mL) is added sodium hydride (5 mg, 0.32 mmol) under argon. The reaction mixture is stirred at rt for 30 min and then transferred to the acid chloride prepared earlier. The reaction mixture is stirred at rt overnight. The reaction mixture is concentrated under vacuum and purified by preparative HPLC (Method 2). Lyophilization of the combined desired fractions affords 7-(7,8-dimethyl-2,4-dioxo-3,4-dihydrobenzo[g]pteridin-10(2H)-yl)-N-(methylsulfonyl)hexanamide (7.4 mg, 11%) as a yellow solid. ¹H NMR (400 MHz, MeOH-d₄) δ 1.53 (m, 2H), 1.68 (m, 2H), 1.85 (m, 2H), 2.16 (t, 2H), 2.44 (s, 3H), 2.57 (s, 3H), 2.92 (s, 3H), 4.71 (t, 2H), 7.75 (s, 1H), 7.92 (s, 1H). LC-MS m/z 434.1 [M+H]⁺, retention time 2.48 min.

### Example 69

### 7-(7,8-Dimethyl-2,4-dioxo-3,4-dihydrobenzo[g]pteridin-10(2H)-yl)-N-(methylsulfonyl)heptanamide

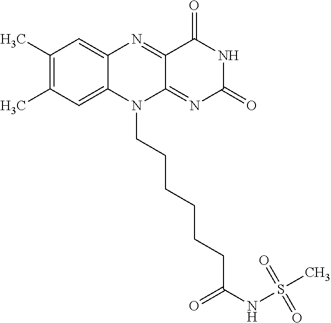

A mixture of 7-(7,8-dimethyl-2,4-dioxo-3,4-dihydrobenzo[g]pteridin-10(2H)-yl)heptanoic acid (52 mg, 0.14 mmol) (prepared using the synthesis of step 3 of Example 25), HATU (70 mg, 0.18 mmol), DIPEA (0.1, 0.57 mmol), and methanesulfonamide (40 mg, 0.42 mmol) in anhydrous DMF (5 mL) are stirred at 25° C. for 3 h. The reaction mixture is concentrated under vacuum and purified by preparative HPLC (Method 2). Lyophilization of the combined desired fractions affords desired product (6.5 mg, 11%) as a yellow solid. ¹H NMR (400 MHz, DMSO-d₆) δ 1.32 (m, 2H), 1.47 (m, 4H), 1.71 (m, 2H), 2.05 (t, 2H), 2.40 (s, 3H), 2.53 (s, 3H), 2.80 (s, 3H), 4.56 (t, 2H), 7.82 (s, 1H), 7.90 (s, 1H), 11.28 (s, 1H). LC-MS m/z 447.9 [M+H]⁺, retention time 4.02 min.

### Example 70

### 10-(2-(3,4-Dichlorobenzylamino)ethyl)-7,8-dimethylbenzo[g]pteridine-2,4(3H,10H)-dione hydrochloride

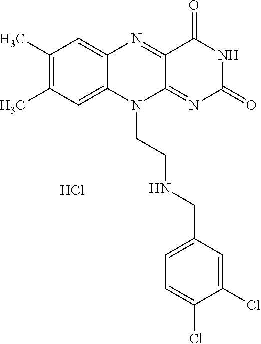

### Step 1 Preparation of 10-(2-(3,4-Dichlorobenzylamino)ethyl)-7,8-dimethylbenzo[g]pteridine-2,4(3H,10H)-dione

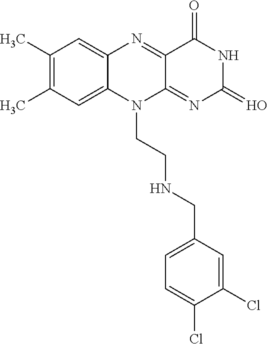

To a solution of (7,8-dimethyl-2,4-dioxo-3,4-dihydro-2H-benzo[g]pteridin-10-yl)-acetaldehyde (Example 14) (44 mg, 0.176 mmol) in ethanol (5 mL) are added (3,4-dichlorophenyl)methanamine (0.1 ml, 0.75 mmol) and AcOH (0.1 ml) at room temperature. The reaction is stirred at 40° C. for 60 min. The reaction mixture is cooled to room temperature and sodium cyanoborohydride (28 mg, 0.44 mmol) is added and the reaction mixture is stirred at rt for 2 h. The solvent is removed under vacuum and the crude is used in the following step without further purification.

### Step 2 Preparation of tert-Butyl 3,4-dichlorobenzyl(2-(7,8-dimethyl-2,4-dioxo-3,4-dihydrobenzo[g]pteridin-10(2H)-yl)ethyl)carbamate

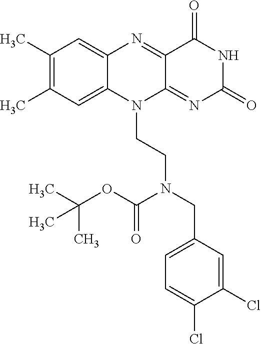

Into a suspension of 10-(2-(3,4-dichlorobenzylamino)ethyl)-7,8-dimethylbenzo[g]pteridine-2,4(3H,10H)-dione in MeOH (5 mL) is added di-tert-butyl dicarbonate (150 mg, 0.69 mmol), and triethylamine (0.1 mL, 0.72 mmol) and the mixture is stirred at room temperature for 16 h. The solvent is removed under vacuum and the crude is purified by preparative TLC (5% MeOH in DCM) to afford desired product (54 mg) as a yellow solid (63% over three steps). LC-MS m/z 543.9 [M+H]⁺, retention time 6.12 min.

### Step 3 Preparation of 10-(2-(3,4-Dichlorobenzylamino)ethyl)-7,8-dimethylbenzo[g]pteridine-2,4(3H,10H)-dione hydrochloride

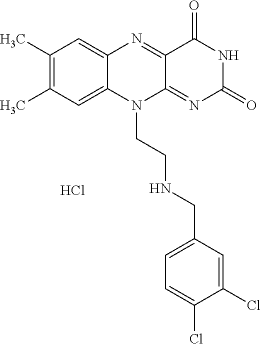

To tert-butyl 3,4-dichlorobenzyl(2-(7,8-dimethyl-2,4-dioxo-3,4-dihydrobenzo[g]pteridin-10(2H)-yl)ethyl)carbamate (54 mg) is added 4 N HCl in dioxane (1 ml) and the mixture is stirred for 2 h at rt. The reaction is monitored by LCMS (Method C). Once the reaction is complete, Et₂O (3 ml) is added and the solid is collected by filtration. The desired product (15.5 mg) is isolated as an HCl salt (Yield: 33%). ¹H NMR (400 MHz, DMSO-_{d6}) δ 2.42 (s, 3H), 2.55 (s, 3H), 3.17 (s, 2H), 4.35 (s, 2H), 4.99 (s, 2H), 7.61 (d, 1H), 7.72 (d, 1H), 7.91 (m, 1H), 7.95 (s, 1H), 8.08 (dd, 1H), 9.55 (s, 2H), 11.46 (s, 1H). LC-MS m/z 443.9 [(M-HCl)+H]⁺, retention time 4.41 min.

### Example 71

### 7,8-Dimethyl-10-(2-(naphthalen-2-ylmethylamino)ethyl)benzo[g]pteridine-2,4(3H,10H)-dione hydrochloride

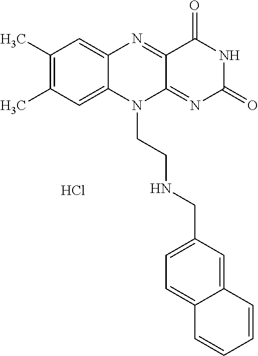

### Step 1 Preparation of 7,8-Dimethyl-10-(2-(naphthalen-2-Ylmethylamino)ethyl)benzo[g]pteridine-2,4(3H,10H)-dione

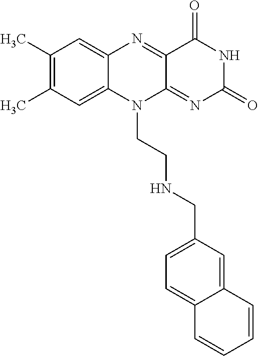

The preparation of this compound is similar to that of Example 70 using 10-(2-aminoethyl)-7,8-dimethylbenzo[g]pteridine-2,4(3H,10H)-dione (Example 40) and 2-naphthaldehyde.

### Step 2 Preparation of 7 tert-Butyl 2-(7,8-dimethyl-2,4-dioxo-3,4-dihydrobenzo[g]pteridin-10(2H)-yl)ethyl(naphthalen-2-ylmethyl)carbamate

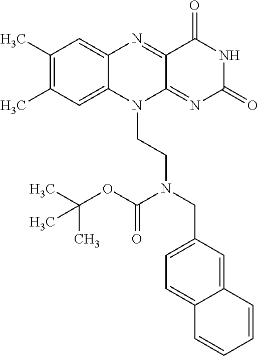

The preparation of this compound is similar to that of Example 70, Step 2.

### Step 3 Preparation of 7,8-Dimethyl-10-(2-(naphthalen-2-ylmethylamino)ethyl)benzo[g]pteridine-2,4(3H,10H)-dione hydrochloride

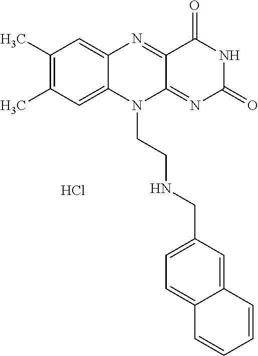

The preparation of this compound is similar to that of Example 70, Step 3. ¹H NMR (400 MHz, DMSO-_{d6}) δ 2.41 (s, 3H), 2.54 (s, 3H), 3.40 (s, 2H), 4.48 (s, 2H), 5.05 (s, 2H), 7.55-7.60 (m, 2H), 7.75 (d, 1H), 7.88-8.10 (m, 4H), 8.13 (m, 1H), 8.18 (s, 1H), 9.76 (s, 2H), 11.46 (s, 1H). LC-MS m/z 426.0 [(M−HCl)+H]⁺, retention time 3.87 min.

### Example 72

### N-Benzyl-N-(2-(7,8-dimethyl-2,4-dioxo-3,4-dihydrobenzo[g]pteridin-10(2H)-yl)ethyl)acetamide

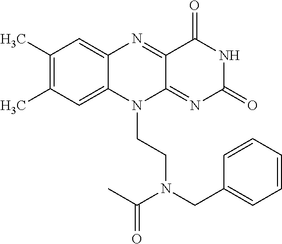

To a solution of 10-(2-(benzylamino)ethyl)-7,8-dimethylbenzo[g]pteridine-2,4(3H,10H)-dione (314 mg, 0.83 mmol) (Example 55) in DCM (10 ml) is added DIPEA (0.45 ml, 2.5 mmol) and acetic anhydride (0.15 ml, 1.58 mmol). The mixture is stirred at rt for 10 min. Solvent is concentrated under reduced pressure to obtain a crude product (147 mg). Crude product (31 mg) is dissolved in MeOH (8 ml) and purified by preparative HPLC (Method 2). Lyophilization of the combined desired fractions affords desired product (13.2 mg, 18% overall yield) as a yellow solid. ¹H NMR (400 MHz, DMSO-d₆) δ 1.95 (s) and 2.22 (s) (rotamers, total of 3H), 2.40 (s) and 2.41 (s) (rotamers, total of 3H), 2.50 (s, 3H), 3.54 (t) and 3.66 (t) (rotamers, total of 2H), 4.55 (s) and 4.70 (s) (rotamers, total of 2H), 4.74 (s, 2H), 7.00-7.40 (m, 5H), 7.65 (s) and 7.85 (s) (rotamers, total of 1H), 7.89 (s, 1H), 11.34 (s) and 11.39 (s) (rotamers, total of 1H). LC-MS m/z 417.9 [M+H]⁺, retention time 4.56 min.

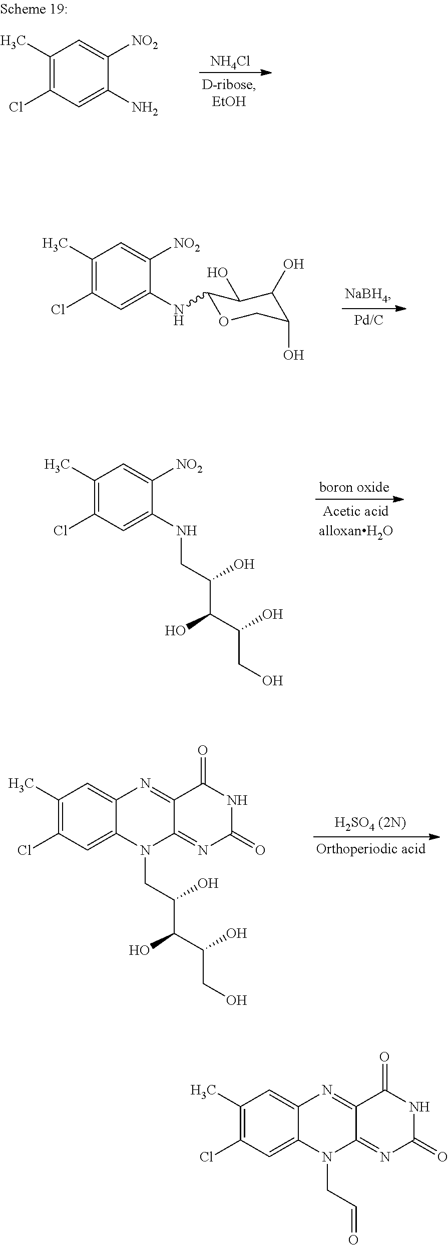

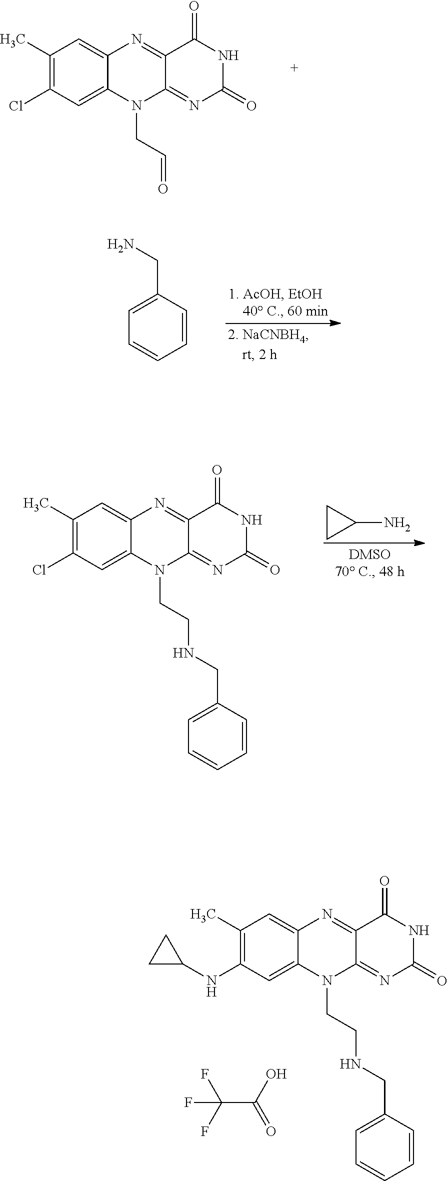

### Example 73

### 10-(2-(Benzylamino)ethyl)-8-(cyclopropylamino)-7-methylbenzo[g]pteridine-2,4(3H,10H)-dione 2,2,2-trifluoroacetate

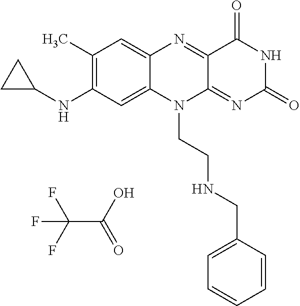

### Step 1 Preparation of 2-(5-Chloro-4-methyl-2-nitro-phenylamino)-tetrahydro-pyran-3,4,5-triol

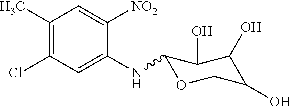

A solution of 5-chloro-4-methyl-2-nitro-phenylamine (19.8 g, 0.1 mol), ammonium chloride (0.1 g), and D-ribose (15.9 g, 0.1 mol) in EtOH (200 mL) is refluxed and stirred over night. The reaction mixture is concentrated under reduced pressure and resuspended in DCM:MeOH (1:1) and the precipitated unreacted staring material is removed by filtration. The mother liquor is dry loaded on silica gel using DCM:MeOH (1:1) and ISCO flash column chromatography is performed. 100% DCM is used until the first peak elutes, then 20% MeOH/DCM is used to elute the 11.5 g of orange product as a sticky solid (Yield: 40%) and 9.58 g of unreacted starting material is recovered. LC-MS m/z 318.7 [M+H], retention time 2.83 min. The product is used in the next step without further purification.

### Step 2 Preparation of 5-(5-Chloro-4-methyl-2-amino-phenylamino)-pentane-1,2,3,4-tetraol

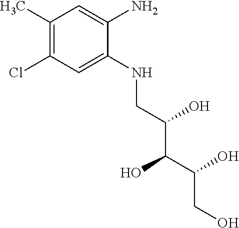

To a solution of 2-(5-chloro-4-methyl-2-nitro-phenylamino)-tetrahydro-pyran-3,4,5-triol (6.87 g, 0.02 mol) in EtOH (125 mL) is added sodium borohydride (1.65 g, 0.043 mol) portionwise such that evolution of gas is controlled to not overflow the contents of the flask. The resulting mixture is heated at reflux for 4 h. The reaction mixture is then cooled to 0° C. at which point Pd/C (300 mg) is added along with additional sodium borohydride (1.65 g, 0.043 mol). The reaction mixture is then allowed to stir at room temperature for 2 h. The reaction is complete when it is observed that the reaction has become colourless. The reaction mixture is filtered through celite and washed liberally with MeOH, and finally concentrated to obtain the crude product, (as a clear purple oil) to be used directly in the next step. LC-MS: m/z 290.9 [M+H], retention time 1.38 min.

### Step 3 Preparation of 8-Chloro-7-methyl-10-(2,3,4,5-tetrahydroxy-pentyl)-10H-benzo[g]pteridine-2,4-dione

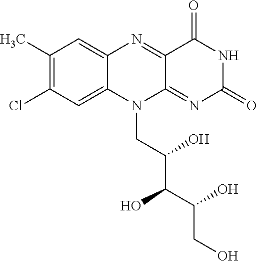

Crude 5-(2-amino-5-chloro-4-methyl-phenylamino)-pentane-1,2,3,4-tetraol (0.022 mol) is dissolved in glacial acetic acid (80 mL), covered in foil, and stirred at room temperature. At which point, the flask is purged with argon for 20 min, and alloxan monohydrate (3.45 g, 0.022 mol), boron trioxide (1.35 g, 0.022 mol) are added to the stirring solution. The reaction is maintained under an argon atmosphere and left to react at room temperature for 3 h and a yellow precipitate is observed in solution. The solution is concentrated under reduced pressure and the residue is dissolved in water (300 mL), put in an ice bath, and the precipitate formed in solution is filtered. The resulting filtrate is purified by preparatory HPLC in 10 mL segments (30 injections) using Method 1. 8-Chloro-7-methyl-10-(2,3,4,5-tetrahydroxy-pentyl)-10H-benzo[g]pteridine-2,4-dione (455 mg) is isolated following lyophilization of the appropriate fractions (Yield: 5.3%). LC-MS m/z 397.1 [M+H], retention time 1.58 min. ¹H NMR (400 MHz, DMSO-d6) δ 2.51 (s, 3H), 3.46 (m, 1H), 3.64 (m, 2H), 4.23 (m, 1H), 4.49 (m, 1H), 4.67 (m, 1H), 4.78 (m, 2 H), 4.88 (m, 1H), 5.15 (m, 2H), 8.13 (s, 1H), 8.20 (s, 1H), 11.47 (s, 1H).

### Step 4 Preparation of (8-Chloro-7-methyl-2,4-dioxo-3,4-dihydro-2H-benzo[g]pteridin-10-yl)-acetaldehyde

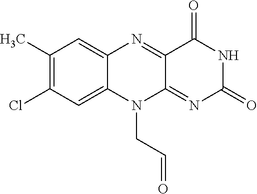

To a cooled suspension (0° C.) of 8-chloro-7-methyl-10-(2,3,4,5-tetrahydroxy-pentyl)-10H-benzo[g]pteridine-2,4-dione (0.235 g, 0.0006 mol) in 2 N aqueous sulfuric acid (60 mL), (in a flask covered with foil), is added (dropwise) orthoperiodic acid (0.41 g, 0.0018 mol), which is dissolved in water (25 mL). After 30 min, the reaction is allowed to warm to rt and is stirred until it becomes a clear, yellow solution. The pH of the reaction solution is then adjusted carefully to 3.8-3.9 (using a pH meter) by addition of solid sodium carbonate [it is extremely important that the pH is monitored carefully, otherwise going over a pH of 3.9 does not allow for the product to precipitate out of solution]. The precipitate is then filtered off and washed liberally with cold water, ethanol, and diethyl ether to yield 0.089 g of the desired product as an orange solid (Yield: 49%). LC-MS m/z 305.1 [M+H] retention time: 1.69 min.

### Step 5 Preparation of 10-(2-(Benzylamino)ethyl)-8-chloro-7-methylbenzo[g]pteridine-2,4(3H,10H)-dione

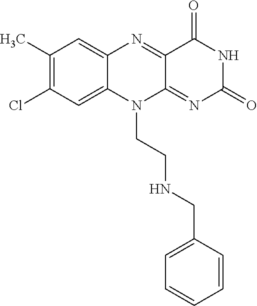

To a solution of (2-(8-chloro-7-methyl-2,4-dioxo-3,4-dihydrobenzo[g]pteridin-10(2H)-yl)acetaldehyde (157 mg, 0.51 mmol) in ethanol (5 mL) is added phenylmethanamine (0.3 ml, 2.75 mmol) and AcOH (0.3 ml) at room temperature. The reaction is stirred at 40° C. for 60 min. The reaction is cooled to room temperature and sodium cyanoborohydride (95 mg, 1.53 mmol) is added and the reaction mixture is stirred at rt for 2 h. The solvent is removed under vacuum and the crudem material (166 mg) is used in the following step without further purification.

### Step 6 Preparation of 10-(2-(Benzylamino)ethyl)-8-(cyclopropylamino)-7-methylbenzo[g]pteridine-2,4(3H,10H)-dione 2,2,2-trifluoroacetate

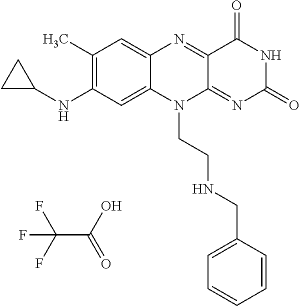

10-(2-(Benzylamino)ethyl)-8-chloro-7-methylbenzo[g]pteridine-2,4(3H,10H)-dione (27 mg, 0.68 mmol) is dissolved in DMSO (4 mL) and then cyclopropylamine (73 mg, 1.28 mmol) is added. The reaction is stirred at 70° C. for 48 h. The reaction mixture is concentrated under vacuum and purified using preparative HPLC (Method 3). 10-(2-(benzylamino)ethyl)-8-(cyclopropylamino)-7-methylbenzo[g]pteridine-2,4(3H,10H)-dione (2.21 mg) is isolated following lyophilization of the appropriate fractions (Yield: 9%). ¹H NMR (400 MHz, CD₃OD) δ 0.83 (m, 2H), 1.10 (m, 2H), 2.21 (s, 3H), 2.82 (m, 1H), 3.89 (s, 2H), 4.48 (s, 2H), 5.11 (s, 2H), 6.90 (s, 1H), 7.01 (s, 1H), 7.43 (m, 3H), 7.55 (m, 2H). LC-MS m/z 417.0 [(M-TFA)+H]⁺, retention time 3.51 min.

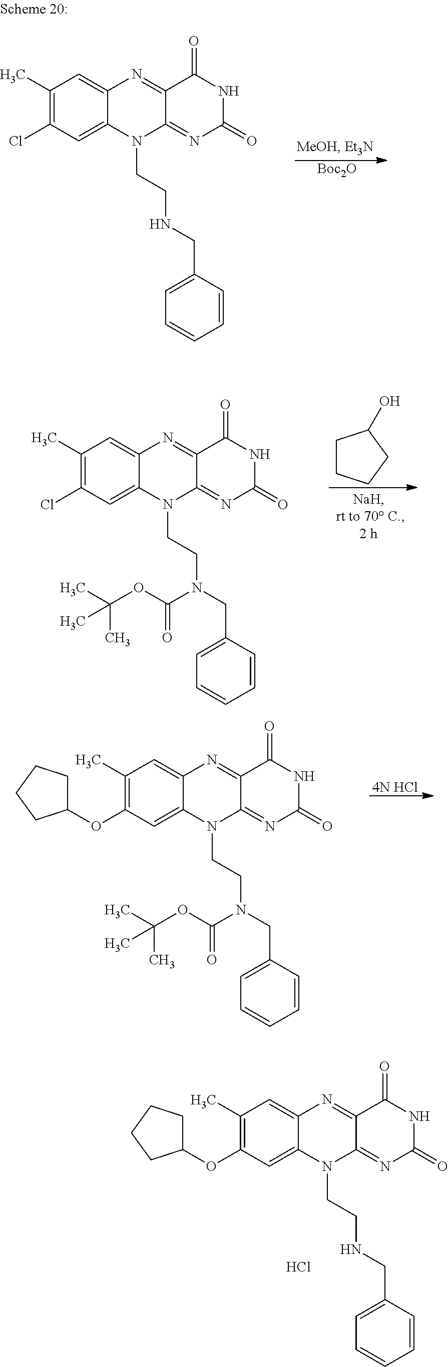

### Example 74

### 10-(2-(benzylamino)ethyl)-8-(cyclopentyloxy)-7-methylbenzo[g]pteridine-2,4(3H,10H)-dione

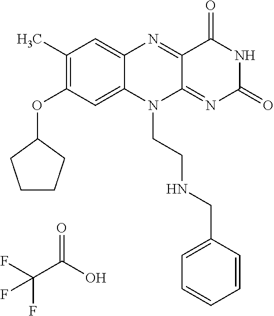

### Step 1 Preparation of tert-Butyl benzyl(2-(8-chloro-7-methyl-2,4-dioxo-3,4-dihydrobenzo[g]pteridin-10(2H)-yl)ethyl)carbamate

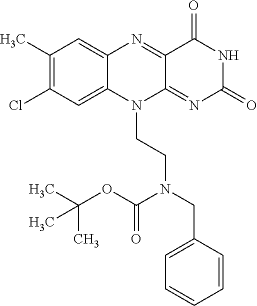

To a solution of crude 10-(2-(benzylamino)ethyl)-8-chloro-7-methylbenzo[g]pteridine-2,4(3H,10H)-dione (Step 5, Example 73) (84 mg, 0.28 mmol) in MeOH (200 mL) is added di-tert-butyl dicarbonate (200 mg, 0.96 mmol) and Et₃N (0.1 mL). The mixture is stirred for 2 h at rt. The reaction is concentrated under reduced pressure and purified via silica gel chromatography (ISCO) (100% DCM to 10% MeOH/DCM) over 1 h to obtain the desired product (45 mg, 43%) as a solid. LC-MS m/z 495.9 [M+H]⁺, retention time 5.19 min.

### Step 2 Preparation of tert-Butyl benzyl(2-(8-(cyclopentyloxy)-7-methyl-2,4-dioxo-3,4-dihydrobenzo[g]pteridin-10(2H)-yl)ethyl)carbamate

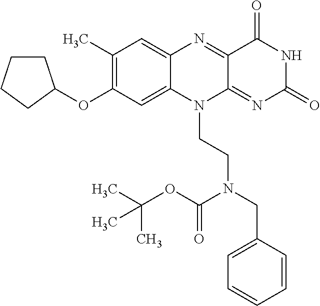

To a solution of NaH (26 mg, 0.65 mmol) in cyclopentanol (3 ml) is added tert-butyl benzyl(2-(8-chloro-7-methyl-2,4-dioxo-3,4-dihydrobenzo[g]pteridin-10(2H)-yl)ethyl)carbamate (43 mg, 0.086 mmol) in cyclopentanol (1 mL). The reaction is stirred at 70° C. for 2 h. The reaction is concentrated under reduced pressure and purified via preparative TLC (5% MeOH in DCM) to afford the desired product (12 mg, 26%) as a solid. LC-MS m/z 545.9 [M+H]⁺, retention time 5.97 min.

### Step 3 Preparation of 10-(2-(benzylamino)ethyl)-8-(cyclopentyloxy)-7-methylbenzo[g]pteridine-2,4(3H,10H)-dione

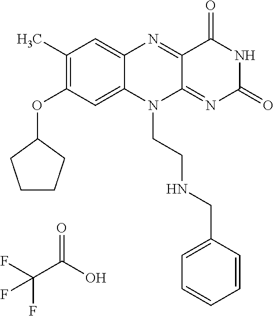

To tert-butyl benzyl(2-(8-(cyclopentyloxy)-7-methyl-2,4-dioxo-3,4-dihydrobenzo[g]pteridin-10(2H)-yl)ethyl)carbamate (12 mg) is added 4 N HCl in dioxane (2 ml) and the mixture is stirred for 2 h at rt. The reaction is monitored by LCMS (Method C). Once the reaction is completed, Et₂O (3 mL) is added and the solid is collected by filtration. The solid is further purified by preparative HPLC (Method 3). Desired product (3.9 mg) is isolated as a yellow solid following lyophilization of the appropriate fractions (Yield: 32%). ¹H NMR (400 MHz, CD₃OD) δ 1.72-1.84 (m, 2H), 1.84-2.04 (m, 4H), 2.08-2.20 (m, 2H), 2.33 (s, 3H), 3.71 (t, 2H), 4.41 (s, 2H), 5.15 (t, 2 H), 5.30 (s, 1H), 7.08 (s, 1H), 7.44 (m, 3H), 7.53 (m, 2H), 7.76 (s, 1H). LC-MS m/z 446.1 [(M-TFA)+H]⁺, retention time 4.35 min.

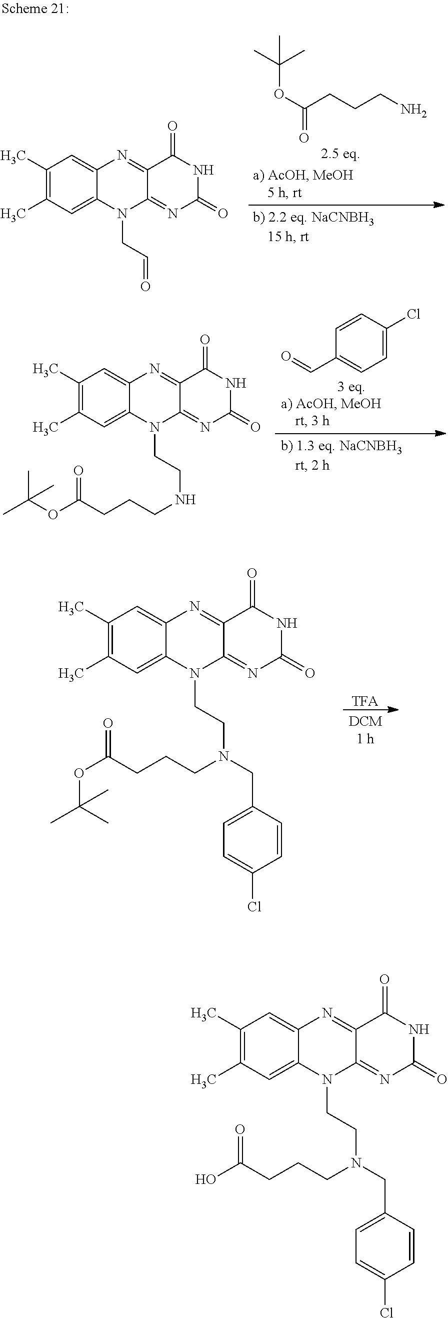

### Example 75

### 4-{(4-Chloro-benzyl)-[2-(7,8-dimethyl-2,4-dioxo-3,4-dihydro-2H-benzo[g]pteridin-10-yl)-ethyl]-amino}-butyric acid

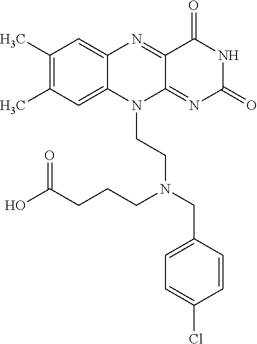

### Step 1: Preparation of 4-[2-(7,8-Dimethyl-2,4-dioxo-3,4-dihydro-2H-benzo[g]pteridin-10-yl)-ethylamino]-butyric acid tert butyl ester

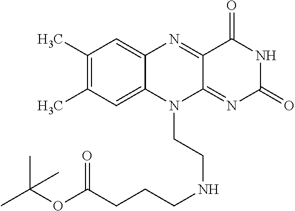

Prepared similarly to Example 20.

### Step 2: Preparation of 4-{(4-Chloro-benzyl)-[2-(7,8-dimethyl-2,4-dioxo-3,4-dihydro-2H-benzo[g]pteridin-10-yl)-ethyl]-amino}-butyric acid tert-butyl ester

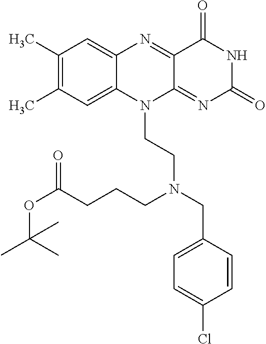

To a suspension of 4-[2-(7,8-dimethyl-2,4-dioxo-3,4-dihydro-2H-benzo[g]pteridin-10-yl)-ethylamino]-butyric acid tert butyl ester (95 mg, 0.22 mmol) in methanol (30 mL) is added 4-chloro-benzaldehyde (94 mg, 0.7 mmol) at room temperature. Glacial acetic acid (10 drops) is added and allowed to stir at room temperature for 3 h. Sodium cyanoborohydride (28 mg, 0.44 mmol) is added, and the solution is stirred for 4 h. The reaction mixture is concentrated, and the residue is dissolved in DMSO, filtered, and purified by preparative HPLC (Method 1). 4-{(4-Chloro-benzyl)-[2-(7,8-dimethyl-2,4-dioxo-3,4-dihydro-2H-benzo[g]pteridin-10-yl)-ethyl]-amino}-butyric acid tert-butyl ester (5.7 mg) is isolated following lyophilization of the appropriate fractions (Yield: 4.0%). ¹H NMR (400 MHz, CD₃OD) δ 1.45 (s, 9 H), 1.68 (m, 2H), 2.23 (t, 2H), 2.49 (s, 6H), 2.68 (t, 2H), 2.91 (m, 2H), 3.46 (s, 2H), 4.8 (m, 2H), 6.78 (d, 2H), 6.96 (d, 2H), 7.56 (s, 1H), 7.93 (s, 1H). LC-MS m/z 552.0 [M+H]⁺, retention time 4.59 min.

### Step 3: Preparation of 4-{(4-Chloro-benzyl)-[1-(7,8-dimethyl-2,4-dioxo-3,4-dihydro-2H-benzo[g]pteridin-10-yl)-ethyl]-amino}-butyric acid

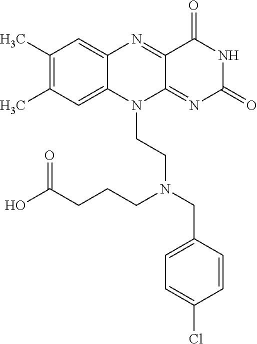

4-{(4-Chloro-benzyl)-[2-(7,8-dimethyl-2,4-dioxo-3,4-dihydro-2H-benzo[g]pteridin-10-yl)-ethyl]-amino}-butyric acid tert-butyl ester (4.6 mg, 0.00832 mmol) is dissolved in a 1:1 mixture of DCM/TFA (3 mL) and allowed to stir at rt for 1 h. The reaction mixture is concentrated and lyophilized without further purification to give 4-{(4-Chloro-benzyl)-[2-(7,8-dimethyl-2,4-dioxo-3,4-dihydro-2H-benzo[g]pteridin-10-yl)-ethyl]-amino}-butyric acid (3.82 mg, Yield: 93%) as a yellow powder. LC-MS m/z 495.9 [M+H]⁺, retention time 3.88 min. ¹H NMR (400 MHz, CD₃OD) δ 2.29 (m, 2H), 2.49 (m, 5H), 2.60 (s, 3H), 3.41 (m, 2H), 3.73 (m, 2H), 4.54 (s, 2H), 5.06 (m, 2H), 7.40 (d, 2 H), 7.58 (d, 2H), 7.72 (s, 1H), 7.92 (s, 1H).

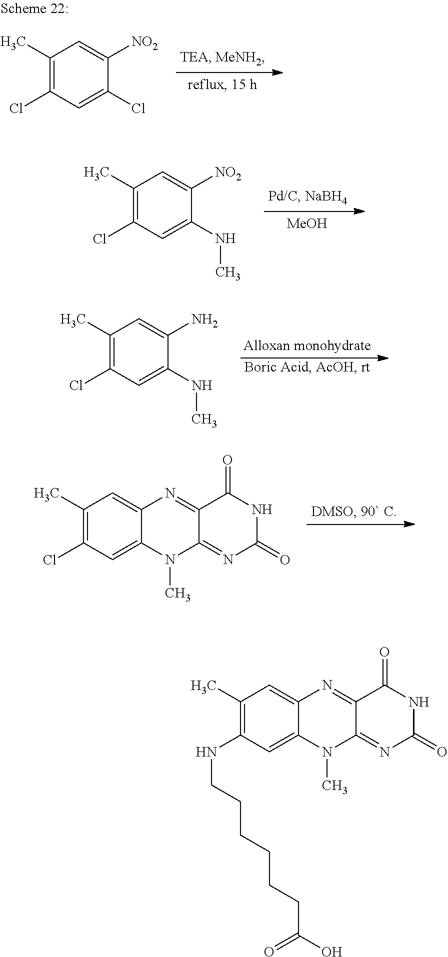

### Example 76

### Preparation of 7-(7,10-Dimethyl-2,4-dioxo-2,3,4,10-tetrahydrobenzo[g]pteridin-8-ylamino)heptanoic acid

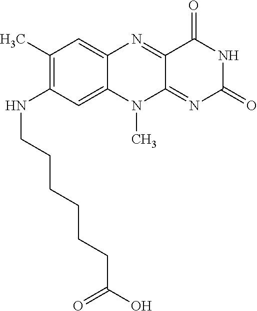

### Step 1 Preparation of 1,5-Dichloro-2-methyl-4-nitro-benzene

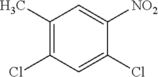

To a solution of 2,4-dichloro-1-methyl-benzene (10.3 g, 0.064 mol) in concentrated sulfuric acid (60 mL) at −10° C., is added nitric acid (2 mL, 0.06 mol) dropwise over 0.5 h, maintaining the reaction temperature at or below −10° C. After the reaction is complete, as monitored by TLC, the reaction mixture is poured into ice (200 mL) and the solid is filtered and washed with water. The crude is recrystallized from hexane and filtered to yield the desired product (10.4 g) as a yellow solid (Yield: 80%).

### Step 2 Preparation of 5-Chloro-NA-dimethyl-2-nitroaniline

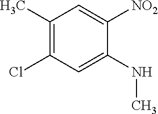

To a solution of 1,5-dichloro-2-methyl-4-nitro-benzene 3.0 g, 13 mmol) in THF (50 mL) is added methylamine hydrochloride (0.94 g, 14 mmol) and triethylamine (10 mL). The reaction mixture is heated to reflux in a sealed flask for 15 h. The reaction mixture is concentrated under reduced pressure and the crude product is purified by flash column chromatography using dichlormethane/hexanes (20%) to DCM (over 20 min) as eluent to yield the desired product (800 mg, 31%). LC-MS m/z 201.1 (M+H), retention time 4.76 min.

### Step 3 Preparation of 5-Chloro-N1,4-dimethylbenzene-1,2-diamine

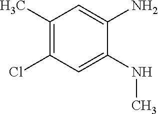

To a suspension of 5-chloro-N,4-dimethyl-2-nitroaniline (0.8 g, 4 mmol) in methanol (40 mL) at room temperature under Ar, is added 10% Pd/C (100 mg), followed by the portionwise addition of sodium borohydride (750 mg, 20 mmol). The reaction is stirred at room temperature for 30 min. After the reaction is complete, as monitored by TLC, the reaction mixture is filtered through a celite pad, which is rinsed with methanol. The filtrate is concentrated under reduced pressure and the residue is used in the next step.

### Step 4 Preparation of 8-Chloro-7,10-dimethylbenzo[g]pteridine-2,4(3H,10H)-dione

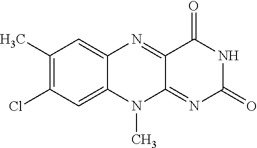

To a solution of 5-chloro-N1,4-dimethylbenzene-1,2-diamine (678 mg, 4 mmol) in acetic acid (25 mL), under an argon atmosphere, is added alloxan monohydrate (640 mg, 4 mmol) and boric acid (494 mg, 8 mmol). The reaction mixture is stirred at rt for 16 h and then is concentrated under reduced pressure. The solid is then suspended in a 1/1 mixture of methanol/water (100 mL) and the precipitate is collected via suction filtration. The precipitate is washed with water (10 mL), isopropanol (10 mL), then diethyl ether (10 mL) to afford crude product (596 mg) by LCMS. The crude product is purified by preparative HPLC (Method 1) to obtain the desired product. LC-MS m/z 277.1 (M+H), retention time 2.33 min.

### Step 5 Preparation of 7-(7,10-Dimethyl-2,4-dioxo-2,3,4,10-tetrahydrobenzo[g]-pteridin-8-ylamino)heptanoic acid

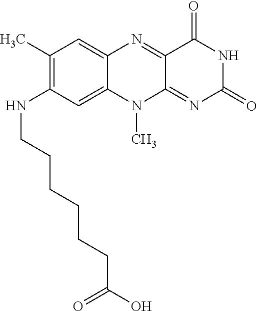

To a solution of 8-chloro-7,10-dimethylbenzo[g]pteridine-2,4(3H,10H)-dione (25 mg, 0.09 mmol) in DMF (5 mL) is added 7-aminoheptanoic acid (78 mg, 0.54 mmol). The reaction mixture is heated at 70° C. for 2 h, then at 90° C. for a further 16 h. The product is purified first using prep TLC (5% MeOH/DCM) then by preparative HPLC (Method 1) to yield the desired product (7.2 mg, 21%). ¹H NMR (400 MHz, DMSO-d₆) δ 1.38 (m, 4H), 1.54 (m, 2H), 1.69 (m, 2H), 2.23 (m, 5H), 3.43 (m, 2H), 3.92 (s, 3H), 6.50 (s, 1H), 7.16 (t, 1H), 7.65 (s, 1H), 10.90 (s, 1H), 11.99 (s, 1H). LC-MS m/z 386.3 (M+H), retention time 2.55 min.

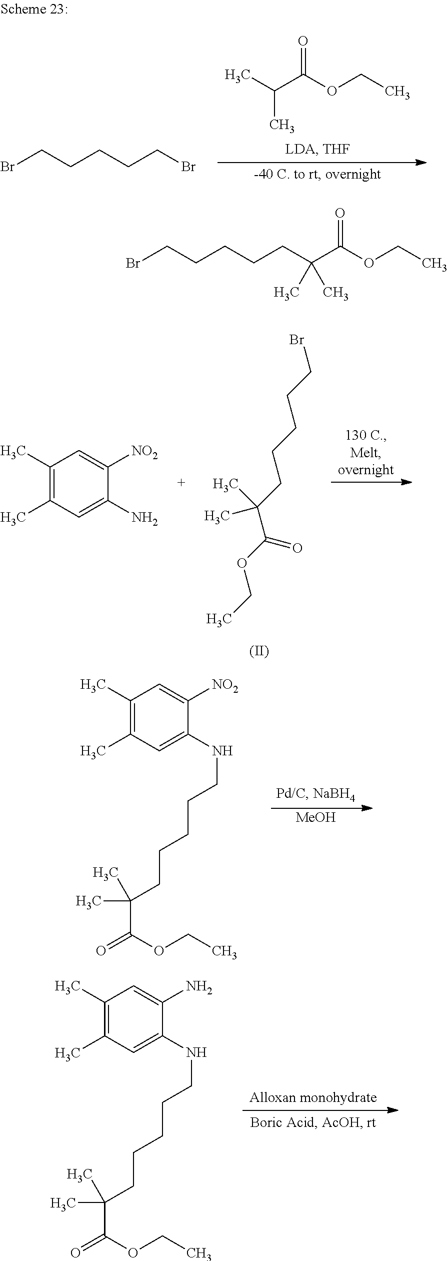

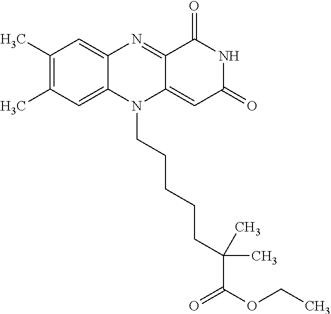

### Example 77

### Ethyl 7-(7,8-dimethyl-2,4-dioxo-3,4-dihydrobenzo[g]pteridin-10(2H)-yl)-2,2-dimethylheptanoate

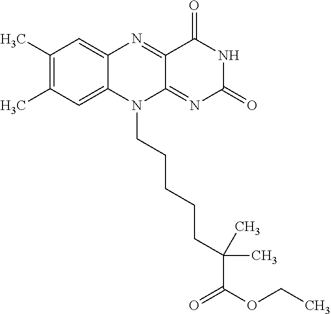

### Step 1 Preparation of Ethyl 7-bromo-2,2-dimethylheptanoate

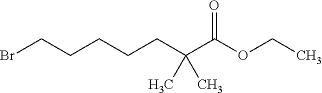

To a stirred solution of ethyl isobutyrate (1.4 g, 12.05 mmol) in anhydrous THF (30 mL) under argon, at −40° C., is added dropwise a solution of lithium diisopropylamide (2.0 M solution in THF, 6.0 mL, 12.05 mmol). After 1 h, dibromopentane (3.95 g, 17.2 mmol) is added. The mixture is stirred for 30 min at −40° C. and then allowed to warm to room temperature and stirred overnight. The reaction is quenched with ice cold water (5 mL). The THF is removed under reduced pressure. The residue is extracted with ethyl acetate (3×20 mL). The combined organic layers are washed with brine, dried over anhydrous Na₂SO₄ and concentrated to afford a crude yellow oil (5.3 g). The residue is purified by silica gel column chromatography using a gradient from hexanes to EtOAc/hexanes (10/90). Desired ethyl 7-bromo-2,2-dimethylheptanoate (2.13 g, Yield: 67%) is obtained as an oil after concentrating the desired fractions under vacuo. ¹H NMR (CDCl₃) δ 1.46 (s, 6H), 1.24 (m, 5H), 1.41 (m, 2H), 1.51 (m, 2H), 1.84 (m, 2H), 3.83 (t, 2H), 4.10 (q, 2H).

### Step 2 Preparation of Ethyl 7-(4,5-dimethyl-2-nitrophenylamino)-2,2-dimethylheptanoate

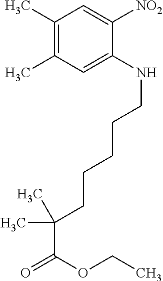

4,5-Dimethyl-2-nitro-phenylamine (332 mg, 2.0 mmol) was melted with ethyl 7-bromo-2,2-dimethylheptanoate (530 mg, 2.0 mmol) in a vial at 130° C. overnight. The reaction is monitored by LCMS. The reaction mixture is dissolved in DCM and purified by silica gel column chromatography using 0 to 30% EtOAc in hexanes as eluent. Desired ethyl 7-(4,5-dimethyl-2-nitrophenylamino)-2,2-dimethylheptanoate (376 mg) is obtained after concentrating the desired fractions under vacuo (Yield: 54%). LC-MS m/z 351.0 [M+H], retention time=6.76 min.

### Step 3 Preparation of Ethyl 7-(2-amino-4,5-dimethylphenylamino)-2,2-dimethylheptanoate

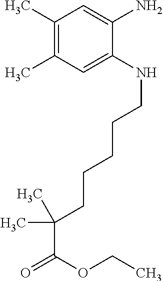

Ethyl 7-(4,5-dimethyl-2-nitrophenylamino)-2,2-dimethylheptanoate (376 mg, 1.07 mmol) is dissolved in methanol (10 mL) and palladium on carbon (10%, 50 mg) is added followed by sodium borohydride (122 mg, 3.22 mmol) with stirring at rt. The reaction mixture is filtered through celite after 30 min and the celite is washed with methanol (10 mL). The filtrate is concentrated under vacuum. The residue is dissolved in water (20 mL), followed by extraction with EtOAc (2×15 mL). The organic layers are combined, dried over sodium sulfate, and filtered. The filtrate is concentrated under vacuo to obtain ethyl 7-(2-amino-4,5-dimethylphenylamino)-2,2-dimethylheptanoate (272 mg) as desired compound (Yield: 80%). LC-MS m/z 321.0 [M+H], retention time=3.73 min.

### Step 4 Preparation of Ethyl 7-(7,8-dimethyl-2,4-dioxo-3,4-dihydrobenzo[g]pteridin-10(2H)-yl)-2,2-dimethylheptanoate

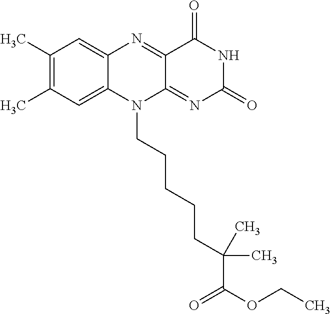

Ethyl 7-(2-amino-4,5-dimethylphenylamino)-2,2-dimethylheptanoate (272 mg, 0.85 mmol) is dissolved in acetic acid (5 mL) followed by addition of boric acid (52 mg, 0.85 mmol) and alloxan monohydrate (136 mg, 0.85 mmol). After 30 min, the reaction mixture is concentrated under vacuum and purified by silica gel column chromatography using 0 to 5% MeOH in DCM as eluent. Ethyl 7-(7,8-dimethyl-2,4-dioxo-3,4-dihydrobenzo[g]pteridin-10(2H)-yl)-2,2-dimethylheptanoate (178 mg, 49%) is obtained after concentrating the desired fractions under vacuo. ¹H NMR (CDCl₃) δ 1.26 (s, 6H), 1.26 (t, 3H), 1.28 (m, 2H), 1.55 (m, 4H), 1.89 (m, 2H), 2.48 (s, 3H), 2.60 (s, 3H), 4.14 (q, 2H), 4.70 (m, 2H), 7.44 (s, 1H), 8.09 (s, 1H), 8.47 (s, 1H). LC-MS m/z 427.1 [M+H], retention time=5.05 min.

### Example 78

### 7-(7,8-Dimethyl-2,4-dioxo-3,4-dihydrobenzo[g]Pteridin-10(2H)-14)-2,2-dimethylheptanoic acid

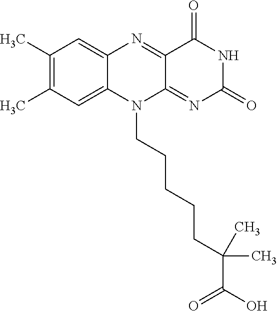

To a solution of ethyl 7-(7,8-dimethyl-2,4-dioxo-3,4-dihydrobenzo[g]pteridin-10(2H)-yl)-2,2-dimethylheptanoate (Example above) (21 mg, 0.049 mmol) in water (2 mL) is added concentrated hydrochloric acid (2 mL). The reaction mixture is stirred at 85° C. for 2 h and then cooled to room temperature, and concentrated under vacuo. The residue is purified by preparative TLC using 15% acetone in DCM as eluent. 7-(7,8-Dimethyl-2,4-dioxo-3,4-dihydrobenzo[g]pteridin-10(2H)-yl)-2,2-dimethylheptanoic acid (14.7 mg, 75%) is isolated by preparative TLC. ¹H NMR (400 MHz, MeOH-d₄) δ 1.18 (s, 6H), 1.42 (m, 2H), 1.60 (m, 4H), 1.94 (m, 2H), 2.57 (s, 3H), 2.70 (s, 3H), 4.86 (m, 2H), 8.08 (s, 1H), 8.15 (s, 1H); LC-MS m/z 399.1 [M+H], retention time 3.07 min.

### Example 79

### 7-(8-(2,3-Dihydroxypropylamino)-7-methyl-2,4-dioxo-3,4-dihydrobenzo[g]pteridin-10(2H)-yl)heptanoic acid

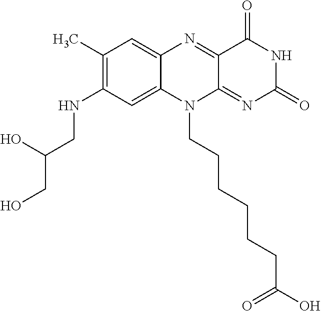

### Step 1 Preparation of tert-Butyl 7-(8-chloro-7-methyl-2,4-dioxo-3,4-dihydrobenzo[g]Pteridin-10(2H)-yl)heptanoate

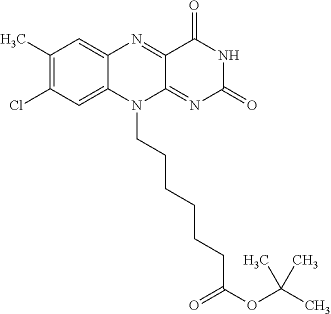

tert-Butyl 7-(8-chloro-7-methyl-2,4-dioxo-3,4-dihydrobenzo[g]pteridin-10(2H)-yl)heptanoate is prepared using the procedure of Example 24 and the appropriately substituted starting materials. LC-MS m/z 446.9, 448.9 (3:1) [M+H], retention time 3.84 min.

### Step 2 Preparation of tert-Butyl 7-(8-(2,3-dihydroxypropylamino)-7-methyl-2A-dioxo-3,4-dihydrobenzo[g]pteridin-10(2H)-yl)heptanoate

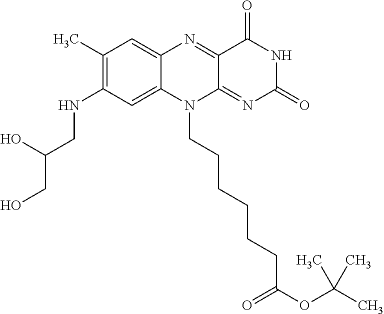

To a suspension of tert-butyl 7-(8-chloro-7-methyl-2,4-dioxo-3,4-dihydrobenzo[g]pteridin-10(2H)-yl)heptanoate (50 mg, 0.11 mmol) in DMSO (3 mL) at room temperature is added 2,3-dihydroxypropylamine (100 mg, 1.1 mmol). The reaction vessel is sealed and the solution is heated to 90° C. with stirring for 16 h. The reaction mixture is then purified by preparative HPLC (Method 1). tert-Butyl 7-(8-(2,3-dihydroxypropylamino)-7-methyl-2,4-dioxo-3,4-dihydrobenzo[g]pteridin-10(2H)-yl)heptanoate is isolated following lyophilization of the appropriate fractions (36 mg, Yield: 65%). ¹H NMR (400 MHz, DMSO-d6) δ 1.38 (s, 9 H), 1.50 (m, 2H), 1.70 (m, 2 H), 2.18 (t, 2H), 2.33 (s, 3H), 3.45 (m, 5H), 3.58 (s, 2H), 3.78 (d, 2H), 4.51 (s, 2H), 4.89 (s, 1H), 5.07 (s, 1H), 6.66 (s, 1H), 7.15 (s, 1H), 7.66 (s, 1H), 10.96 (s, 1H). LC-MS m/z 502.0 [M+H], retention time 2.77 min.

### Step 3: Preparation of 7-(8-(2,3-Dihydroxypropylamino)-7-methyl-2,4-dioxo-3,4-dihydrobenzo[g]pteridin-10(2H)-yl)heptanoic acid

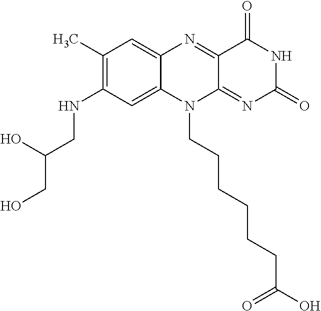

tert-Butyl 7-(8-(2,3-dihydroxypropylamino)-7-methyl-2,4-dioxo-3,4-dihydrobenzo[g]pteridin-10(2H)-yl)heptanoate (19 mg, 0.038 mmol) is suspended in 4 M aqueous HCl (4 mL). The reaction vessel is sealed and the solution is heated to 50° C. with stirring for 4 h. The reaction mixture is lyophilized to give 7-(8-(2,3-dihydroxypropylamino)-7-methyl-2,4-dioxo-3,4-dihydrobenzo[g]pteridin-10(2H)-yl)heptanoic acid (12 mg, Yield: 72%). ¹H NMR (400 MHz, DMSO-d6) δ 1.58 (m, 6H), 1.70 (m, 2H), 2.25 (m, 5H), 3.45 (m, 3H), 3.62 (m, 3H), 3.78 (s, 1H), 4.51 (s, 2H), 6.68 (s, 1H), 7.22 (s, 1H), 7.67 (s, 1H), 11.00 (s, 1H). LC-MS m/z 446.2 [M+H], retention time 2.01 min.

### Example 80

### 7-(8-(Cyclopentylmethyl)amino)-7-methyl-2,4-dioxo-3,4-dihydrobenzo[g]pteridin-10(2H)-yl)heptanoic acid

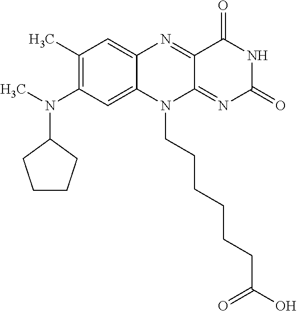

### Step 1 Preparation of tert-Butyl 7-(8-(cyclopentyl(methyl)amino)-7-methyl-2,4-dioxo-3,4-dihydrobenzo[g]pteridin-10(2H)-yl)heptanoate

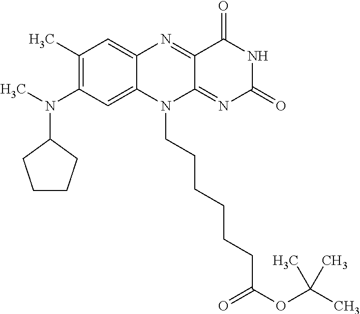

Prepared by a procedure similar to that of Example 79, Step 1, using tert-butyl 7-(8-chloro-7-methyl-2,4-dioxo-3,4-dihydrobenzo[g]pteridin-10(2H)-yl)heptanoate (Example 79, Step 1) (50 mg) and N-methylcyclopentanamine (109 mg, 10 equivalents) in DMSO (3 ml). Purified by preparative HPLC (Method 1). tert-Butyl 7-(8-(cyclopentyl(methyl)amino)-7-methyl-2,4-dioxo-3,4-dihydrobenzo[g]pteridin-10(2H)-yl)heptanoate is isolated following lyophilization of the appropriate fractions (11 mg, Yield: 20%). ¹H NMR (400 MHz, DMSO-d6) δ 1.38 (s, 9 H), 1.51 (m, 7 H), 1.70 (m, 6 H), 1.85 (m, 2H), 2.18 (t, 2H), 2.41 (s, 3H), 2.87 (s, 3H), 4.07 (m, 2H), 4.58 (s, 2H), 6.99 (s, 1H), 7.83 (s, 1H), 11.14 (s, 1H). LC-MS m/z 510.2 [M+H], retention time 4.28 min.

### Step 2 Preparation of 7-(8-(Cyclopentyl(methyl)amino)-7-methyl-2,4-dioxo-3,4-dihydrobenzo[g]pteridin-10(2H)-yl)heptanoic acid

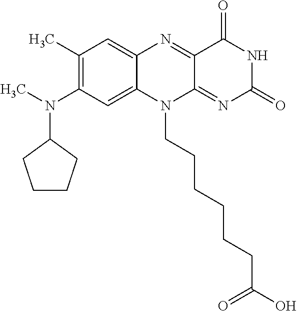

Prepared by a procedure similar to that of Example 79 using tert-butyl 7-(8-(cyclopentyl(methyl)amino)-7-methyl-2,4-dioxo-3,4-dihydrobenzo[g]pteridin-10(2H)-yl)heptanoate (8 mg, 0.016 mmol). 7-(8-(cyclopentyl(methyl)amino)-7-methyl-2,4-dioxo-3,4-dihydrobenzo[g]pteridin-10(2H)-yl)heptanoic acid (4 mg, Yield: 57%) is obtained after lyophilization. ¹H NMR (400 MHz, DMSO-d₆) δ 1.48 (m, 8 H), 1.70 (m, 6H), 1.86 (m, 2H), 2.20 (t, 2H), 2.42 (s, 3H), 2.88 (s, 3H), 4.06 (m, 1H), 4.58 (s, 2H), 6.99 (s, 1 H), 7.83 (s, 1H), 11.14 (s, 1H), 12.00 (s, 1H). LC-MS m/z 454.3 [M+H], retention time 3.26 min.

### Example 81

### 7-(7-Methyl-2,4-dioxo-84(2S,3R,4R,5R)-2,3,4,5,6-pentahydroxyhexylamino)-3,4-dihydrobenzo[g]pteridin-10(2H)-yl)heptanoic acid

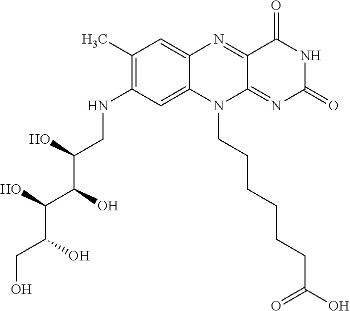

### Step 1 Preparation of tert-Butyl 7-(7-methyl-2,4-dioxo-84(2S,3R,4R,5R)-2,3,4,5,6-pentahydroxyhexylamino)-3,4-dihydrobenzo[g]pteridin-10(2H)-yl)heptanoate

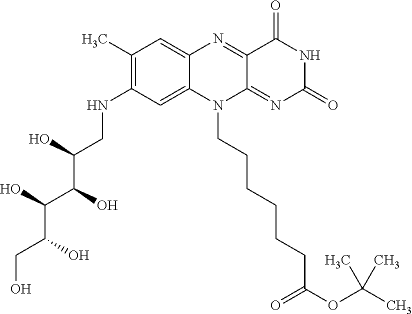

Prepared by nucleophilic aromatic substitution by a procedure similar to that of Example 79, using tert-butyl 7-(8-chloro-7-methyl-2,4-dioxo-3,4-dihydrobenzo[g]pteridin-10(2H)-yl)heptanoate (Example 79, Step 1)(50 mg) and (2R,3R,4R,5S)-6-aminohexane-1,2,3,4,5-pentanol (199 mg, 10 equivalents) in DMSO (3 ml), and purified by preparative HPLC (Method 1). tert-Butyl 7-(7-methyl-2,4-dioxo-8-((2S,3R,4R,5R)-2,3,4,5,6-pentahydroxyhexylamino)-3,4-dihydrobenzo[g]pteridin-10(2H)-yl)heptanoate (37 mg, Yield: 57%) is isolated following lyophilization of the appropriate fractions. ¹H NMR (400 MHz, DMSO-d6) δ 1.43 (m, 15 H), 1.71 (m, 2H), 2.18 (t, 2H), 2.27 (s, 3H), 2.33 (m, 1H), 2.67 (m, 1H), 3.64 (m, 8 H), 3.91 (m, 2H), 4.55 (m, 3H), 6.67 (s, 1H), 7.21 (m, 1H), 7.66 (s, 1H), 10.95 (s, 1H). LC-MS m/z 592.1 [M+H], retention time 2.56 min.

### Step 2 Preparation of 7-(7-Methyl-2,4-dioxo-8-((2S,3R,4R,5R)-2,3,4,5,6-pentahydroxyhexylamino)-3,4-dihydrobenzo[g]pteridin-10(2H)-yl)heptanoic acid

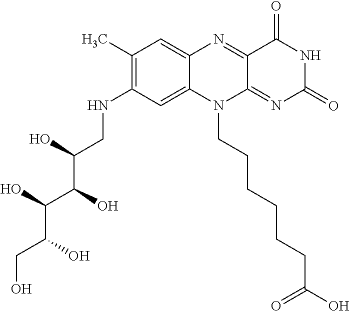

Prepared from tert-butyl 7-(7-methyl-2,4-dioxo-8-((2S,3R,4R,5R)-2,3,4,5,6-pentahydroxyhexylamino)-3,4-dihydrobenzo[g]pteridin-10(2H)-yl)heptanoate (33 mg, 0.056 mmol) by suspending starting material in 4 N HCl in dioxane (5 ml) with stirring at room temperature for 1 h. The product is precipitated using Et₂O then centrifuged and the supernatant is decanted, this process is then repeated three times, with the final pellet being dried under reduced pressure to give 7-(7-methyl-2,4-dioxo-8-((2S,3R,4R,5R)-2,3,4,5,6-pentahydroxyhexylamino)-3,4-dihydrobenzo[g]pteridin-10(2H)-yl)heptanoic acid (23 mg, Yield: 75%). ¹H NMR (400 MHz, DMSO-d6) δ 1.35 (m, 2H), 1.51 (m, 4H), 1.60 (s, 3H), 1.72 (m, 2H), 2.22 (t, 2H), 2.34 (s, 3H), 3.42 (m, 1H), 3.61 (t, 1H), 3.75 (m, 2H), 3.94 (m, 1H), 4.57 (d, 2H), 5.11 (br s, 5H), 6.83 (s, 1H), 7.78 (s, 1H), 7.85 (s, 1H), 11.67 (s, 1H). LC-MS m/z 536.2 [M+H], retention time 3.27 min.

### Example 82

### 7-(8-(3-(4-(3-Aminopropylamino)butylamino)propylamino)-7-methyl-2,4-dioxo-3,4-dihydrobenzo[g]pteridin-10(2H)-yl)heptanoic acid

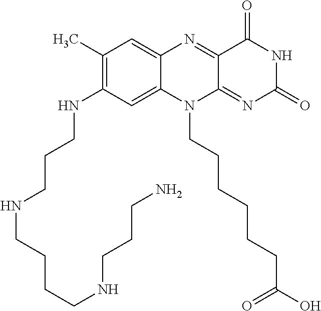

### Step 1: Preparation of tert-Butyl 7-(8-(3-(4-(3-aminopropylamino)butylamino)propylamino)-7-methyl-2,4-dioxo-3,4-dihydrobenzo[g]pteridin-10(2H)-yl)heptanoate

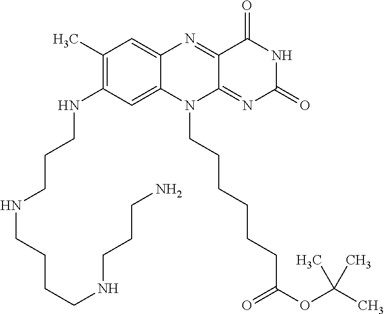

N,N′-Bis(3-aminopropyl)-1,4-butanediamine tetrahydrochloride (383 mg, 1.1 mmol) is suspended in dry DMSO (2 ml) at room temperature. NaH (52 mg, 2.2 mmol, pre-washed 3× with hexanes) is added carefully and the mixture is allowed to stir at room temperature for 20 minutes. tert-Butyl 7-(8-chloro-7-methyl-2,4-dioxo-3,4-dihydrobenzo[g]pteridin-10(2H)-yl)heptanoate (Example 79, Step 1) (50 mg, 0.11 mmol) is then added to the reaction vessel and dry DMSO (1 ml) is used to wash down all reagents from the sides of the flask. The reaction mixture is then stirred at 90° C. for 16 h. After cooling, the reaction mixture is purified by preparative HPLC (Method 1) to give tert-Butyl 7-(8-(3-(4-(3-aminopropylamino)butylamino)propylamino)-7-methyl-2,4-dioxo-3,4-dihydrobenzo[g]pteridin-10(2H)-yl)heptanoate after lyophilization of the appropriate fractions (45 mg, Yield: 57%). ¹H NMR (400 MHz, DMSO-d6) δ 1.38 (s, 9 H), 1.42 (m, 2H), 1.51 (quint., 2H), 1.61 (br s, 3H), 1.72 (m, 2H), 1.86 (m, 2H), 1.99 (m, 2H), 2.08 (s, 1H), 2.19 (t, 2H), 2.28 (s, 3H), 2.94 (m, 9 H), 3.54 (d, 3H), 4.58 (m, 2 H), 6.50 (s, 1H), 7.16 (m, 1H), 7.70 (s, 1H), 7.85 (m, 1H), 8.67 (m, 3H), 10.99 (s, 1H). LC-MS m/z 613.3 [M+H], retention time 2.26 min.

### Step 2 Preparation of 7-(8-(3-(4-(3-Aminopropylamino)butylamino)propylamino)-7-methyl-2,4-dioxo-3,4-dihydrobenzo[d]pteridin-10(2H)-yl)heptanoic acid

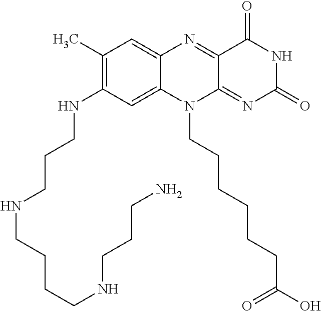

Prepared using tert-butyl 7-(8-(3-(4-(3-aminopropylamino)butylamino)propylamino)-7-methyl-2,4-dioxo-3,4-dihydrobenzo[g]pteridin-10(2H)-yl)heptanoate (33 mg, 0.054 mmol) in the same manner as in Example 81 to give 7-(8-(3-(4-(3-aminopropylamino)butylamino)propylamino)-7-methyl-2,4-dioxo-3,4-dihydrobenzo[g]pteridin-10(2H)-yl)heptanoic acid (27 mg, Yield: 90%). ¹H NMR (400 MHz, DMSO-d6) δ 1.38 (m, 2H), 1.50 (m, 3H), 1.60 (s, 1H), 1.74 (br s, 5H), 2.00 (m, 3H) 2.23 (t, 2H), 2.33 (br s, 2H), 2.95 (m, 7H), 3.43 (m, 1H), 3.66 (s, 2H), 4.20 (br s, 5H), 4.63 (s, 2H), 6.59 (s, 1H), 7.60 (s, 1H), 7.74 (s, 1H), 8.10 (s, 2H), 9.23 (d, 3H), 11.26 (s, 1H). LC-MS m/z 557.3 [M+H], retention time 3.27 min.

### Example 83

### 7-(8-(Cyclopentyloxy)-7-methyl-2,4-dioxo-3,4-dihydrobenzo[g]pteridin-10(2H)-yl)heptanoic acid

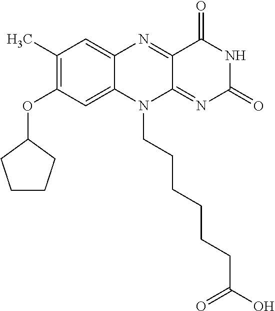

NaH (26 mg, 1.1 mmol) is added to a flask containing cyclopentanol (4 ml), and the resulting mixture is allowed to stir at room temperature for 20 minutes. tert-Butyl 7-(8-chloro-7-methyl-2,4-dioxo-3,4-dihydrobenzo[g]pteridin-10(2H)-yl)heptanoate (Example 79, Step 1) (50 mg, 0.11 mmol) is then added to the reaction vessel and the reaction mixture is stirred at 100° C. for 16 h. After cooling, the reaction mixture is quenched with acetic acid and the mixture is evaporated under reduced pressure to give a dark solid. The residue is purified by preparative HPLC (using an aqueous phase containing USP water with no TFA, acetonitrile as the organic phase, and a gradient from 100% aqueous to 98% organic over 29 minutes). After lyophilization of the appropriate fractions, 7-(8-(cyclopentyloxy)-7-methyl-2,4-dioxo-3,4-dihydrobenzo[g]pteridin-10(2H)-yl)heptanoic acid is obtained (5 mg, Yield: 10%). ¹H NMR (400 MHz, DMSO-d6) δ 1.36 (m, 2H), 1.49 (m, 4H), 1.72 (m, 6H), 1.83 (m, 2H), 2.03 (m, 2H), 2.18 (t, 2H), 2.26 (s, 3H), 4.64 (m, 2H), 5.30 (m, 1H), 7.10 (s, 1H), 7.90 (s, 1H), 11.22 (s, 1H). LC-MS m/z 441.1 [M+H], retention time 5.31 min.

### Example 84

### 7-(8-(Cyclopentylmethoxy)-7-methyl-2,4-dioxo-3,4-dihydrobenzo[g]pteridin-10(2H)-yl)heptanoic acid

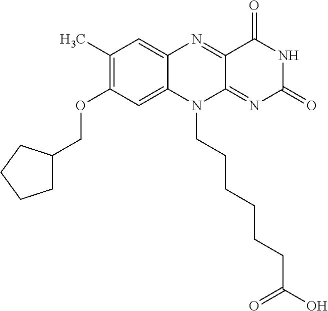

NaH (26 mg, 1.1 mmol) is added to a flask containing cyclopentylmethanol (110 mg, 1.1 mmol) in dry DMSO (1 ml), and the resulting mixture is allowed to stir at room temperature for 20 minutes. tert-Butyl 7-(8-chloro-7-methyl-2,4-dioxo-3,4-dihydrobenzo[g]pteridin-10(2H)-yl)heptanoate (Example 79, Step 1) (50 mg, 0.11 mmol) is then added to the reaction vessel with dry DMSO (1 ml) and the reaction mixture is stirred at 90° C. for 3 h. After cooling, the reaction mixture is quenched with acetic acid and the volatiles are removed by evaporation under reduced pressure. The resulting oily mixture is purified first by preparative TLC using a mobile phase consisting of 10% MeOH in DCM. A mixture is obtained containing the product and various impurities, which is then repurified by preparative TLC using a mobile phase consisting of 5% MeOH in DCM, and preparative HPLC (using an aqueous phase containing USP water with no TFA, acetonitrile as the organic phase, and a gradient from 100% aqueous to 98% organic over 29 minutes) was used for the final purification. After lyophilization of the appropriate fractions, 7-(8-(cyclopentylmethoxy)-7-methyl-2,4-dioxo-3,4-dihydrobenzo[g]pteridin-10(2H)-yl)heptanoic acid is obtained (4.2 mg, Yield: 9%). ¹H NMR (400 MHz, DMSO-d6) δ 1.38 (t, 2H), 1.45 (m, 4H), 1.51 (m, 2H), 1.59 (m, 2H), 1.64 (m, 2H), 1.73 (m, 2H), 1.84 (m, 2H), 2.21 (t, 2H), 2.30 (s, 3H), 2.43 (quin., 1H), 4.21 (d, 2H), 4.65 (t, 2H), 7.15 (s, 1H), 7.92 (s, 1H), 11.22 (s, 1H), 12.00 (br.s, 1H). LC-MS m/z 455.2 [M+H], retention time 5.67 min.

### Example 85

### 7-(4-(10-(6-Carboxyhexyl)-7-methyl-2,4-dioxo-2,3,4,10-tetrahydrobenzo[g]pteridin-8-yl)piperazin-1-yl)-1-cyclopropyl-6-fluoro-4-oxo-1,4-dihydroquinoline-3-carboxylic acid

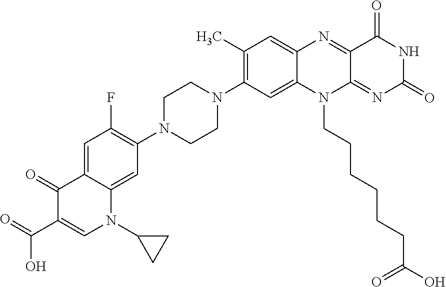

### Step 1 Preparation of 7-(4-(10-(7-tert-Butoxy-7-oxoheptyl)-7-methyl-2,4-dioxo-2,3,4,10-tetrahydrobenzo[g]pteridin-8-yl)piperazin-1-yl)-1-cyclopropyl-6-fluoro-4-oxo-1,4-dihydroquinoline-3-carboxylic acid

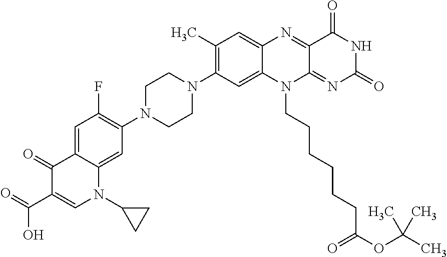

1-Cyclopropyl-6-fluoro-4-oxo-7-(piperazin-1-yl)-1,4-dihydroquinoline-3-carboxylic acid (33 mg, 1.0 mmol) is suspended in dry DMSO (1 ml), and NaH (5 mg, 0.2 mmol) is added carefully with stirring at room temperature. After 20 minutes, tert-butyl 7-(8-chloro-7-methyl-2,4-dioxo-3,4-dihydrobenzo[g]pteridin-10(2H)-yl)heptanoate (Example 79, Step 1) (50 mg, 0.11 mmol) and diisopropylethylamine (0.019 ml, 0.2 mmol) are added in DMSO (2 ml), and the reaction mixture is heated to 90° C. for 40 h, then 115° C. for 4 h. The mixture is cooled and acidified with conc. AcOH (0.5 ml) and then with conc. HCl, added dropwise, to pH 4.5, and extracted with DCM (10 ml) four times. The combined organic fractions are washed successively with water acidified to pH 4.5 (10 mL) and brine, and then evaporated. The resulting mixture is purified by preparative HPLC (Method 1), and the product is precipitated from the mobile phase upon standing overnight. The product is collected by filtration, washed with water, then collected by dissolving the product cake with DCM, and then removing the solvent under reduced pressure to give 7-(4-(10-(7-tert-butoxy-7-oxoheptyl)-7-methyl-2,4-dioxo-2,3,4,10-tetrahydrobenzo[g]pteridin-8-yl)piperazin-1-yl)-1-cyclopropyl-6-fluoro-4-oxo-1,4-dihydroquinoline-3-carboxylic acid (13 mg, Yield: 18%). NMR (400 MHz, DMSO-d6) δ 1.00 (m, 1H), 1.23 (m, 2H), 1.31 (m, 2H), 1.36 (s, 9H), 1.37 (q, 2H), 1.48 (m, 4H), 1.76 (m, 2H), 2.19 (t, 2H), 3.44-3.86 (m, 6H), 4.63 (s, 2H), 6.76 (s, 1H), 7.20 (s, 1H), 7.69 (d, 1H), 7.96 (m, 2H), 8.70 (s, 1H), 11.24 (s, 1H). LC-MS m/z 742.1 [M+H], retention time 5.65 min.

### Step 2 Preparation of 7-(4-(10-(6-Carboxyhexyl)-7-methyl-2,4-dioxo-2,3,4,10-tetrahydrobenzo[g]pteridin-8-11)piperazin-1-yl)-1-cyclopropyl-6-fluoro-4-oxo-1,4-dihydroquinoline-3-carboxylic acid

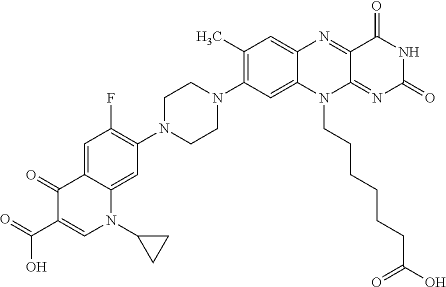

7-(4-(10-(7-tert-Butoxy-7-oxoheptyl)-7-methyl-2,4-dioxo-2,3,4,10-tetrahydrobenzo[g]pteridin-8-yl)piperazin-1-yl)-1-cyclopropyl-6-fluoro-4-oxo-1,4-dihydroquinoline-3-carboxylic acid (6.5 mg, 0.0088 mmol) is suspended in TFA (1 ml) and DCM (1 ml), and is stirred overnight at room temperature. After evaporating the solvent, the resulting solid is dissolved in a minimum amount of hot DMF, precipitated with USP water and collected by filtration, then loaded onto a preparative TLC plate and eluted with 10% MeOH in DCM with 0.1% AcOH to give 7-(4-(10-(6-carboxyhexyl)-7-methyl-2,4-dioxo-2,3,4,10-tetrahydrobenzo[g]pteridin-8-yl)piperazin-1-yl)-1-cyclopropyl-6-fluoro-4-oxo-1,4-dihydroquinoline-3-carboxylic acid (4.1 mg, Yield: 68%). ¹H NMR (400 MHz, DMSO-d6) δ 0.85 (m, 2H), 1.07 (m, 1H), 1.23 (m, 5H), 1.45 (m, 4H), 1.72 (s, 3H), 2.01 (m, 2H), 2.73 (s, 1H), 2.89 (s, 1H), 4.63 (m, 2H), 7.22 (m, 1H), 7.50 (m, 1H), 7.68 (m, 1H), 8.00 (m, 2H), 8.60 (m, 1H), 11.20 (m, 1H). LC-MS m/z 686.1 [M+H], retention time 5.72 min.

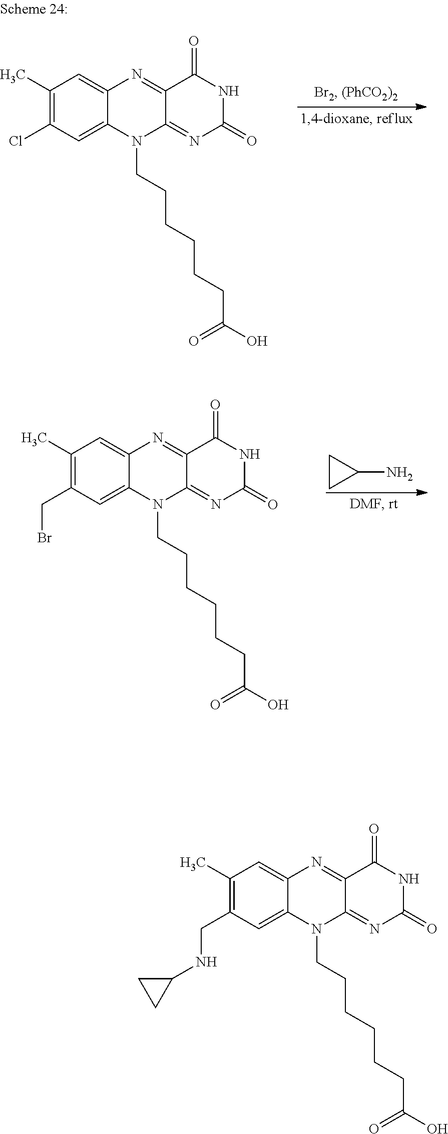

### Example 86

### 7-(8-((Cyclopropylamino)methyl)-7-methyl-2,4-dioxo-3,4-dihydrobenzo[g]pteridin-10(2H)-yl)heptanoic acid

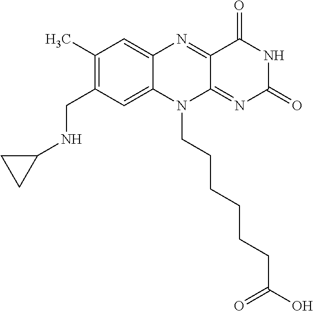

### Step 1 Preparation of 7-(8-(Bromomethyl)-7-methyl-2,4-dioxo-3,4-dihydrobenzo[2]pteridin-10(2H)-yl)heptanoic acid

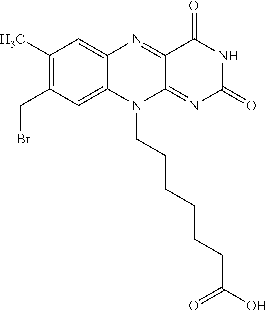

A suspension of 7-(7,8-dimethyl-2,4-dioxo-3,4-dihydrobenzo[g]pteridin-10(2H)-yl)heptanoic acid (Example 25, Step 3) (370 mg, 1 mmol) in 1,4-dioxane (10 mL) is heated to reflux. Two solutions are prepared: Br₂ (351 mg, 2.2 mmol) in 1,4-dioxane (4 ml); and benzoyl peroxide (121 mg, 0.5 mmol) in 1,4-dioxane (1 ml). A portion of the benzoyl peroxide solution (0.5 ml) is added in one portion to the refluxing mixture, followed by a portion-wise addition of the entirety of the bromine mixture over 15 minutes. At 15 minutes, an additional portion of the benzoyl peroxide solution (0.25 ml) is added, and the remainder (0.25 ml) is added after a further 30 minutes. The reaction is removed from the heat 70 minutes after the initial addition, cooled to room temperature and the solvent is removed under reduced pressure. Upon the addition of CHCl₃ (40 ml) a brown solid precipitates from solution, which is filtered and washed with CHCl₃ (3×10 ml) and dried under reduced pressure to obtain 7-(8-(bromomethyl)-7-methyl-2,4-dioxo-3,4-dihydrobenzo[g]pteridin-10(2H)-yl)heptanoic acid (201 mg, Yield: 45%) as a brown powdery solid which is used without further purification. LC-MS m/z 448.9/450.9 (1:1) [M+H], retention time 4.18 min.

### Step 2 Preparation of 7-(84(Cyclopropylamino)methyl)-7-methyl-2,4-dioxo-3,4-dihydrobenzo[g]pteridin-10(2H)-yl)heptanoic acid

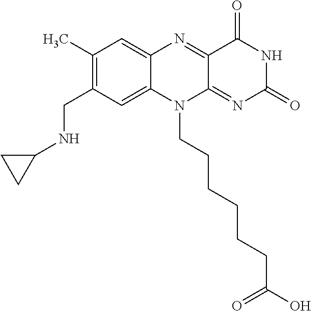

7-(8-(Bromomethyl)-7-methyl-2,4-dioxo-3,4-dihydrobenzo[g]pteridin-10(2H)-yl)heptanoic acid (35 mg, 0.08 mmol) and cyclopropylamine (9 mg, 0.16 mmol) are suspended in DMF (1 ml) and stirred at room temperature for 16 h. The reaction mixture is then purified by preparative HPLC (Method 1, using methanol in place of aceonitrile in the organic phase). 7-(84(Cyclopropylamino)methyl)-7-methyl-2,4-dioxo-3,4-dihydrobenzo[g]pteridin-10(2H)-yl)heptanoic acid is obtained after lyophilization of the appropriate fractions (7.5 mg, Yield: 22%). ¹H NMR (400 MHz, DMSO-d₆) δ 0.86 (d, 4H), 1.37 (d, 2H), 1.52 (m, 4H), 1.76 (m, 2H), 2.23 (t, 2H), 2.55 (s, 3H), 2.84 (m, 1H), 4.51 (m, 4H), 7.92 (s, 1H), 8.04 (s, 1H), 9.26 (br s, 1H), 11.40 (s, 1H). LC-MS m/z 426.0 [M+H], retention time 3.76 min.

### Example 87

### 7-(84(2,3-Dihydroxypropylamino)methyl)-7-methyl-2,4-dioxo-3,4-dihydrobenzo[g]pteridin-10(2H)-yl)heptanoic acid 2,2,2-trifluoroacetate

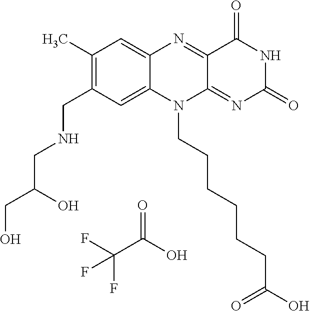

Prepared using a procedure similar to that of Example 86 using 7-(8-(bromomethyl)-7-methyl-2,4-dioxo-3,4-dihydrobenzo[g]pteridin-10(2H)-yl)heptanoic acid (35 mg, 0.08 mmol) and 2,3-dihydroxypropylamine (15 mg, 0.16 mmol) in DMF (1 ml). 7-(8-((2,3-Dihydroxypropylamino)methyl)-7-methyl-2,4-dioxo-3,4-dihydrobenzo[g]pteridin-10(2H)-yl)heptanoic acid 2,2,2-trifluoroacetate is obtained after lyophilization of the appropriate fractions (5.7 mg, Yield: 17%). ¹H NMR (400 MHz, DMSO-d6) δ 1.46 (m, 6H), 1.75 (m, 2H), 2.22 (t, 2H), 2.52 (s, 3H), 3.00 (t, 1H), 3.24 (m, 2H), 3.90 (m, 1H), 4.45 (s, 2H), 4.54 (s, 2H), 4.98 (br s, 1H), 5.60 (s, 1H), 7.99 (s, 1H), 8.02 (s, 1H), 9.14 (br s, 2H), 11.40 (s, 1H), 12.02 (br s, 1H). LC-MS m/z 460.3 [M+H], retention time 1.77 min.

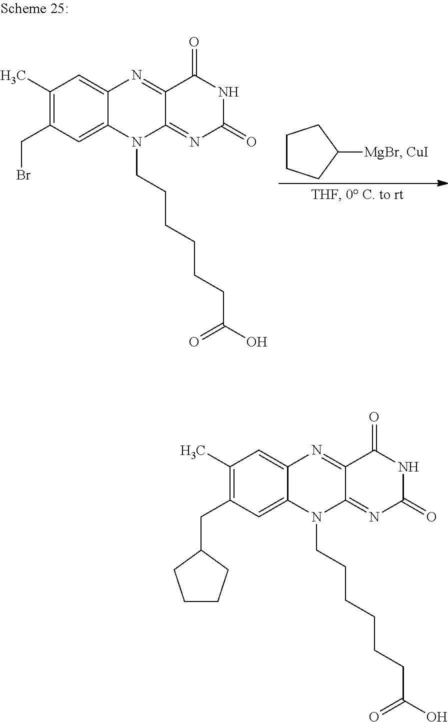

### Example 88

### 7-(8-(Cyclopentylmethyl)-7-methyl-2,4-dioxo-3,4-dihydrobenzo[g]pteridin-10(2H)-yl)heptanoic acid

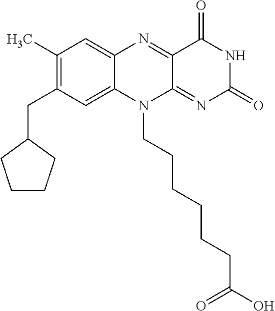

Into an oven-dried vial is added CuI (10 mg, 0.052 mmol), cyclopentyl magnesium bromide (2.0 M, 110 μl, 0.22 mmol) and anyhydrous THF (1 ml) at 0° C. under argon. 7-(8-(Bromomethyl)-7-methyl-2,4-dioxo-3,4-dihydrobenzo[g]pteridin-10(2H)-yl)heptanoic acid (Example 86, step 1)) (50 mg, 0.11 mmol) is dissolved in dry THF (4 ml), then added to the reaction vial. The reaction mixture is allowed to warm to room temperature over 16 h, and then quenched with NH₄Cl. After extracting the aqueous phase with DCM, the combined organic layers are basified and extracted with 2M NaOH (3×10 ml). The alkaline aqueous phase is washed with EtOAc (2×10 ml), then acidified with 2M HCl to pH<3 and extracted with DCM (3×20 ml). The organic phase is then washed with brine (2×20 ml), dried with Na₂SO₄, filtered and evaporated. The residue is purified by preparative HPLC (Method 1). 7-(8-(Cyclopentylmethyl)-7-methyl-2,4-dioxo-3,4-dihydrobenzo[g]pteridin-10(2H)-yl)heptanoic acid is obtained after lyophilization of the appropriate fractions (1.1 mg, Yield: 2%). ¹H NMR (400 MHz, CD₃OD) δ 0.90 (m, 2H), 1.31 (m, 4H), 1.45 (q, 2H), 1.59 (m, 5H), 1.76 (m, 2H), 1.87 (t, 2H), 2.27 (m, 2H), 2.50 (s, 3H), 2.94 (t, 2H), 4.75 (m, 2H), 7.69 (s, 1H), 7.98 (s, 1H). LC-MS m/z 439.1 [M+H], retention time 4.92 min.

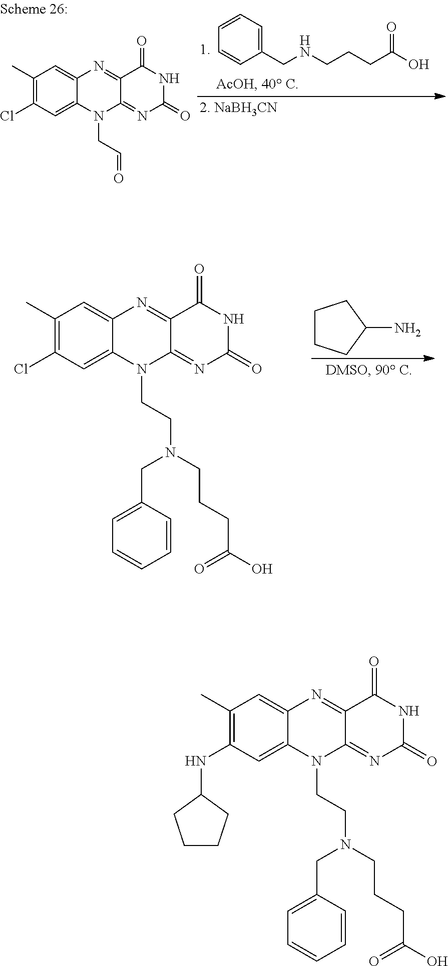

### Example 89

### 4-(Benzyl(2-(8-(cyclopentylamino)-7-methyl-2,4-dioxo-3,4-dihydrobenzo[g]pteridin-10(2H)-yl)ethyl)amino)butanoic acid

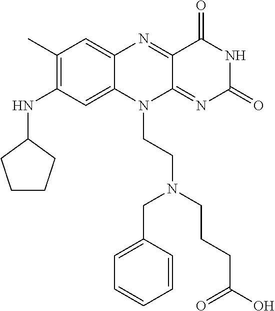

### Step 1: Preparation of 4-(Benzyl(2-(8-chloro-7-methyl-2,4-dioxo-3,4-dihydrobenzo[g]pteridin-10(211)-yl)ethyl)amino)butanoic acid

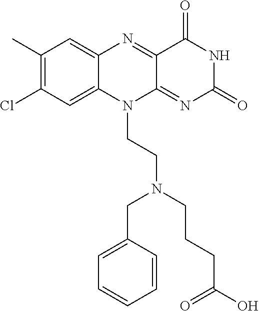

4-(Benzyl(2-(8-chloro-7-methyl-2,4-dioxo-3,4-dihydrobenzo[g]pteridin-10(2H)-yl)ethyl)amino)butanoic acid is prepared in the same manner as Example 15, Step 2 starting from commercially available 4-(benzylamino)butanoic acid and (8-chloro-7-methyl-2,4-dioxo-3,4-dihydro-2H-benzo[g]pteridin-10-yl)-acetaldehyde (Example 73, Step 4) LC-MS m/z 481.9, 483.9 (3:1) [M+H], retention time 3.71 min.

### Step 2 Preparation of 4-(Benzyl(2-(8-(cyclopentylamino)-7-methyl-2,4-dioxo-3,4-dihydrobenzo[g]pteridin-10(211)-yl)ethyl)amino)butanoic acid

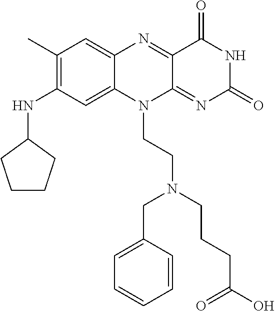

4-(Benzyl(2-(8-(cyclopentylamino)-7-methyl-2,4-dioxo-3,4-dihydrobenzo[g]pteridin-10(2H)-yl)ethyl)amino)butanoic acid is prepared from 4-(benzyl(2-(8-chloro-7-methyl-2,4-dioxo-3,4-dihydrobenzo[g]pteridin-10(2H)-yl)ethyl)amino)butanoic acid and cyclopentylamine in the same manner as Example 79, Step 2. LC-MS m/z 531.1 [M+H], retention time 4.27 min.

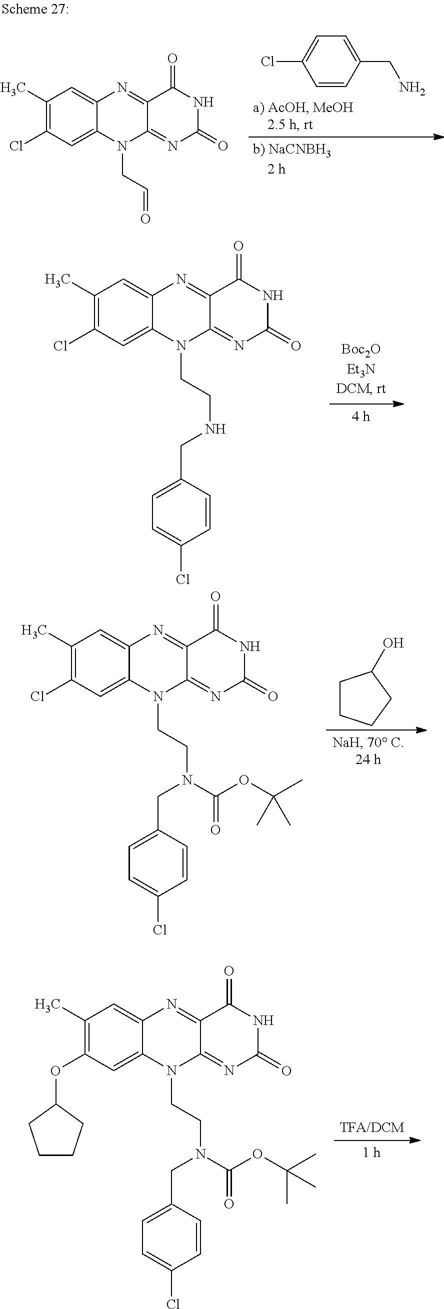

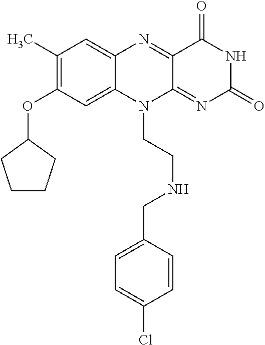

### Example 90

### 10-[2-(4-Chloro-benzylamino)-ethyl]-8-cyclopentyloxy-7-methyl-10H-benzo[g]pteridine-2,4-dione

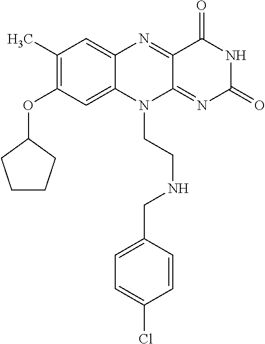

### Step 1 Preparation of 8-Chloro-10-[2-(4-chloro-benzylamino)-ethyl]-7-methyl-10H-benzo[g]pteridine-2,4-dione

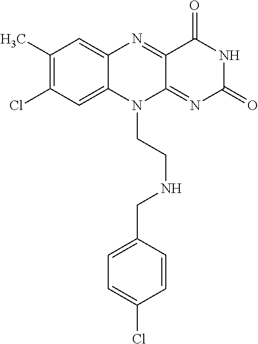

Prepared similarly to Example 73, Step 5.

### Step 2 Preparation of (4-Chloro-benzyl)-[2-(8-chloro-7-methyl-2,4-dioxo-3,4-dihydro-2H-benzo[g]pteridin-10-yl)-ethyl]-carbamic acid tert-butyl ester

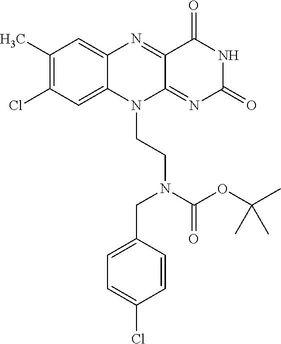

Prepared similarly to Example 74, Step 1.

### Step 3 Preparation of (4-Chloro-benzyl)-[2-(8-cyclopentyloxy-7-methyl-2,4-dioxo-3,4-dihydro-2H-benzo[g]pteridin-10-yl)-ethyl]-carbamic acid tert-butyl ester

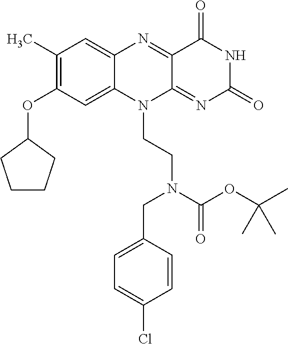

Prepared similarly to Example 74, Step 2.

### Step 4 Preparation of 10-[2-(4-Chloro-benzylamino)-ethyl]-8-cyclopentyloxy-7-methyl-10H-benzo[g]pteridine-2,4-dione

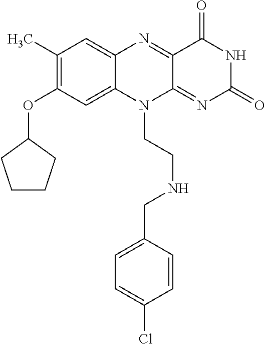

(4-Chloro-benzyl)-[2-(8-cyclopentyloxy-7-methyl-2,4-dioxo-3,4-dihydro-2H-benzo[g]pteridin-10-yl)-ethyl]-carbamic acid tert-butyl ester (3.98 mg, 0.00686 mmol) is dissolved in a 1:1 mixture of DCM/TFA (3 mL) and stirred at rt for 1 h. The reaction mixture is concentrated and lyophilized without further purification to give 10-[2-(4-chloro-benzylamino)-ethyl]-8-cyclopentyloxy-7-methyl-10H-benzo[g]pteridine-2,4-dione (4.03 mg, Yield: 100%) as a yellow powder. ¹H NMR (400 MHz, CD₃OD) δ 1.80 (m, 2H), 1.95 (m, 4H), 2.12 (m, 2H), 2.34 (s, 3H), 3.69 (m, 2H), 4.40 (s, 2H), 5.14 (t, 2H), 5.30 (m, 1H), 7.01 (s, 1H), 7.48 (dd, 2H), 7.51 (dd, 2H), 7.87 (s, 1H). LC-MS m/z 480.0 [M+H]⁺, retention time 5.03 min.

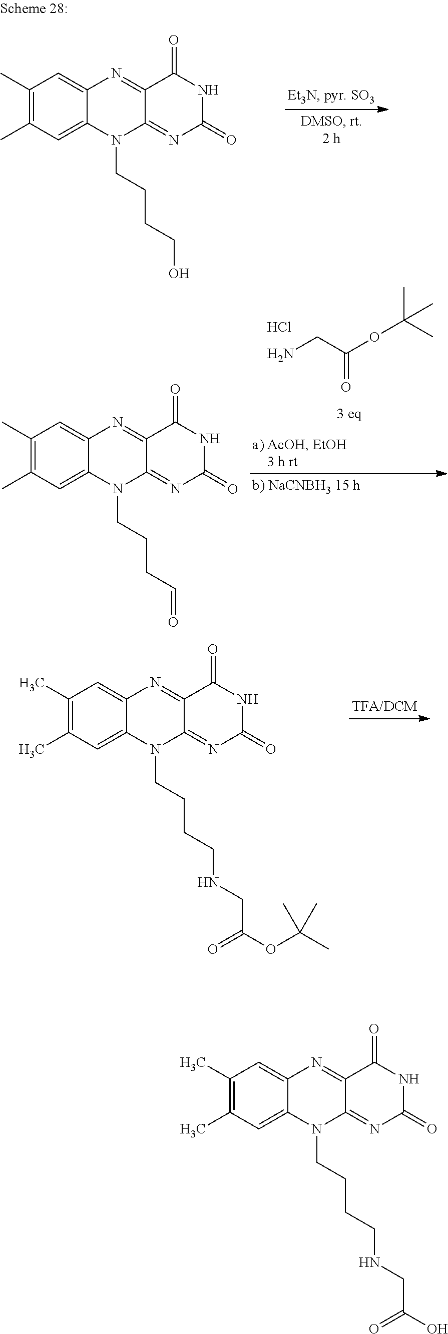

### Example 91

### [4-(7,8-Dimethyl-2,4-dioxo-3,4-dihydro-2H-benzo[g]pteridin-10-yl)-butylamino]-acetic acid

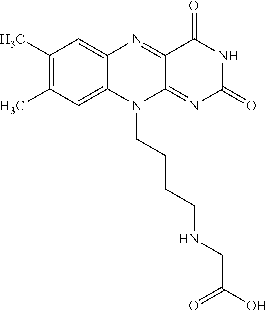

### Step 1 Preparation of 4-(7,8-Dimethyl-2,4-dioxo-3,4-dihydro-2H-benzo[g]pteridin-10-yl)-butyraldehyde

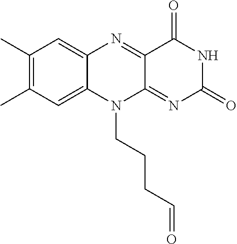

10-(4-Hydroxy-butyl)-7,8-dimethyl-10H-benzo[g]pteridine-2,4-dione (prepared similarly to Example 77, Steps 2-4 150 mg, 0.48 mmol) is dissolved in DMSO (5 mL) and then triethylamine (145 mg, 1.44 mmol) is added and stirred at rt. After 45 minutes, pyridine sulfur trioxide complex is added to the reaction mixture and the mixture is stirred at rt for an additional hour. The crude reaction mixture dissolved in DMSO is carried on to the next step.

### Step 2 Preparation of [4-(7,8-Dimethyl-2,4-dioxo-3,4-dihydro-2H-benzo[g]pteridin-10-yl)-butylamino]-acetic acid tert-butyl ester

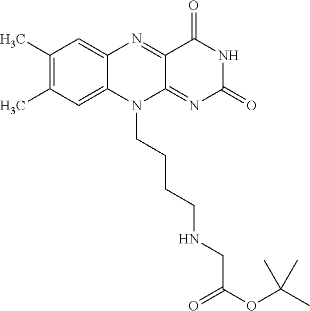

Prepared similarly to Example 73, Step 5.

### Step 3 Preparation of [4-(7,8-Dimethyl-2,4-dioxo-3,4-dihydro-2H-benzo[E]pteridin-10-yl)-butylamino]-acetic acid

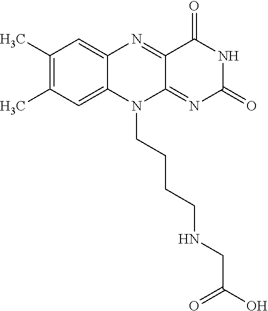

[4-(7,8-Dimethyl-2,4-dioxo-3,4-dihydro-2H-benzo[g]pteridin-10-yl)-butylamino]-acetic acid tert-butyl ester (8.0 mg, 0.0187 mmol) is dissolved in a 1:1 mixture of DCM/TFA (3 mL) and stirred at rt for 1 h. The reaction mixture is concentrated and lyophilized without further purification to give [4-(7,8-dimethyl-2,4-dioxo-3,4-dihydro-2H-benzo[g]pteridin-10-yl)-butylamino]-acetic acid (6.47 mg, Yield: 93%) as a yellow powder. ¹H NMR (400 MHz, CD₃OD) δ 1.92 (m, 2H), 2.01 (m, 2H), 2.48 (s, 3H), 2.60 (s, 3H), 3.27 (m, 2H), 3.93 (s, 2H), 4.78 (t, 2H), 7.80 (s, 1H), 7.97 (s, 1H). LC-MS m/z 372.1 [M+H]⁺, retention time 3.41 min.

### Example 92

### tert-Butyl 7-(8-Cyclopropyl-7-methyl-2,4-dioxo-3,4-dihydrobenzo[g]pteridin-10(2H)-yl)heptanoate

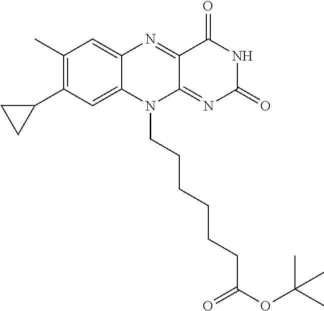

### Step 1 Preparation of 4-Amino-2-cyclopropyl-5-nitrotoluene

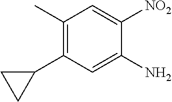

A well-stirred slurry of 4-amino-2-chloro-5-nitrotoluene (500 mg, 2.7 mmol), cyclopropylboronic acid (460 mg, 5.4 mmol) and Cs₂CO₃ (2.60 g, 8.0 mmol) in anhydrous 1,4-dioxane (10.0 mL) is sparged with nitrogen for 10 min. [1,1′-Bis(diphenylphosphino)ferrocene]dichloropalladium(II), complex with dichloromethane (1:1) (440 mg, 0.54 mmol) is added and sparging continued for another 10 min. The reaction is sealed under nitrogen and heated at 90° C. for 24 h. The reaction is cooled, diluted with DCM (100 mL) and filtered through Celite®. The organics are washed with saturated bicarbonate solution, brine, dried with anhydrous sodium sulfate and concentrated. The residue is chromatographed on silica gel (Silicycle, 230-400 mesh, 150 g, elution with 10% ethyl acetate/hexane) to give 290 mg of an orange solid. ¹H NMR (400 MHz, CDCl₃) δ ppm 0.68 (2H, m), 1.02 (2H, m), 1.86 (1H, m), 2.31 (3H, s), 5.91 (2H, br s), 6.32 (1H, s), 7.88 (1H, s). MS (ESI+) for C₁₀H₁₂N₂O₂ m/z 193.2 (M+H)⁺, retention time: 4.17 min (System C).

### Step 2 Preparation of tert-Butyl 7-[(5-cyclopropyl-4-methyl-2-nitrophenyl)amino]heptanoate

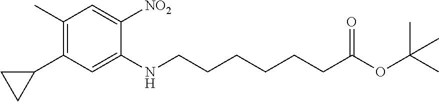

A solution of 4-amino-2-cyclopropyl-5-nitrotoluene (190.0 mg, 0.9885 mmol) in dry DMF (5.0 mL) is cooled at 0° C. under nitrogen and sodium hydride (39.5 mg, 0.988 mmol) is added as a solid. Hydrogen evolution is observed and the mixture is allowed to warm to rt and stir for 30 min. tert-Butyl 7-bromoheptanoate (314 mg, 1.19 mmol) in DMF (2 mL) is then added dropwise via syringe and stirring is continued at rt for 18 h. The reaction is concentrated in vacuo to remove DMF and the residue is partitioned between DCM and saturated ammonium chloride (25 mL each). The layers are separated, the aqueous is extracted with DCM (3×25 mL), and the organics are combined, dried with anhydrous sodium sulfate and concentrated. The residue is chromatographed on silica gel (Silicycle, 230-400 mesh, 50 g, elution with 5% EtOAc/Hexane) to give 120 mg of desired product as an orange solid. ¹H NMR (400 MHz, CDCl₃) 5 ppm 0.70 (2H, m), 1.04 (2H, m), 1.42 (4H, m), 1.44 (9 H, s), 1.62 (2H, m), 1.71 (2H, m), 1.88 (1H, m), 2.23 (2H, t), 2.32 (3H, s), 3.25 (2H, m), 6.35 (1H, s), 7.93 (1H, s), 7.98 (1H, br s.). MS (ESI+) for C₂₁H₃₂N₂O₄ m/z 399.2 (M+Na)⁺, retention time: 6.13 min (System C).

### Step 3 Preparation of tert-Butyl 7-[(2-amino-5-cyclopropyl-4-methylphenyl)amino]heptanoate

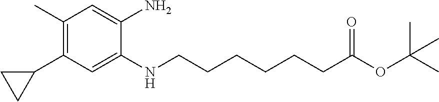

A solution of tert-butyl 7-[(5-cyclopropyl-4-methyl-2-nitrophenyl)amino]heptanoate (120.0 mg, 0.3187 mmol) in ethanol (5.0 mL) is stirred at rt and activated (wet) Raney Nickel (20 mg, 0.3 mmol) is added. The reaction is then placed under an atmospheric pressure of hydrogen gas and evacuated and purged 4 times prior to allowing the reaction to stir at rt. After 18 h, the mixture is filtered through Celite® and concentrated to give 110 mg of desired product as a clear, colorless oil that is used immediately without further purification. MS (ESI+) for C₂₁H₃₄N₂O₂ m/z 347.3 (M+H)+, retention time: 3.92 min (System C).

### Step 4 Preparation of tert-Butyl 7-(8-Cyclopropyl-7-methyl-2,4-dioxo-3,4-dihydrobenzo[g]pteridin-10(2H)-0)heptanoate

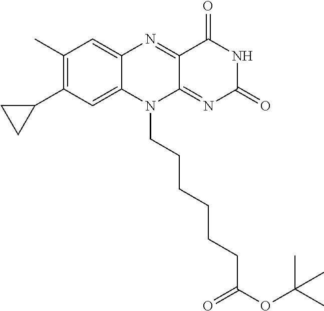

To a well-stirred mixture of tert-butyl 7-[(2-amino-5-cyclopropyl-4-methylphenyl)amino]heptanoate (110.0 mg, 0.32 mmol) and boron oxide (23.9 mg, 0.64 mmol) in acetic acid (1 mL) at rt under nitrogen is added alloxan (50.8 mg, 0.32 mmol) and the reaction is heated at 60° C. for 60 min. The residue is partitioned between DCM and saturated sodium bicarbonate solution (30 mL each) and the layers are separated. The aqueous layer is extracted with DCM (3×30 mL) and the organics are combined, dried with anhydrous sodium sulfate and concentrated. The residue is chromatographed on silica gel (Silicycle, 230-400 mesh, 50 g, elution with 2% EtOH in chloroform) to give 37 mg of desired product as an amorphous yellow solid. ¹H NMR (400 MHz, DMSO-d6) δ ppm 0.93 (2H, m), 1.14 (2H, m), 1.39 (9H, s), 1.41 (4H, m), 1.51 (2H, m), 1.66 (2H, m), 2.19 (3H, m), 2.55 (3H, s), 4.61 (2H, m), 7.22 (1H, s), 7.91 (1H, s), 11.29 (1H, s). MS (ESI+) for C₂₅H₃₂N₄O₄ m/z 453.2 (M+H)+, retention time: 4.23 min (System C).

By using the methods described above and by selecting the appropriate starting materials, other compounds of the invention are prepared and characterized. These compounds, together with the Examples described above, are summarized in Table 1. The invention therefore encompasses and claims each and all compounds in Table 1 collectively and/or separately as well as compositions containing the same, methods of treatment comprising administering such compound(s) and uses of these compound(s) in the menufacture of a medicament for the prophylaxis or treatment of the disorders as hereinbefore described.

TABLE 1

| | | | LC/MS | | | |
|---|---|---|---|---|---|---|
| | | | re- | | | |
| | | LC-MS | tention | | | |
| En- | | MH+ | time | LC/MS | | |
| try | Structures | (m/z) | (min) | method | Preparation | Name |
| 1 | 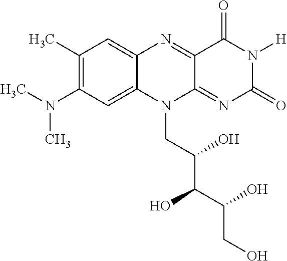 | | | | | 8-(dimethylamino)-7- methyl-10-((2S,3S,4R)- 2,3,4,5- tetrahydroxypentyl)benzo[g] pteridine-2,4(3H,10H)-dione |
| 2 | 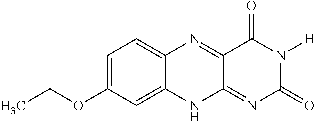 | | | | | 8-ethoxybenzo[g]pteridine- 2,4(3H,10H)-dione |
| 3 | 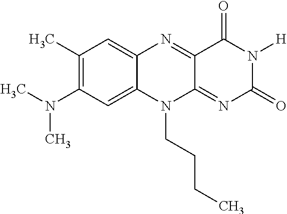 | 328.3 | 5.9 | Method A | Prepared using the synthesis of Example 10 | 10-butyl-8- (dimethylamino)-7- methylbenzo[g]pteridine- 2,4(3H,10H)-dione |
| 4 | 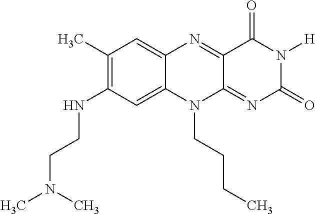 | 371.2 | 4.9 | Method A | Prepared using the synthesis of Example 10 | 10-butyl-8-(2- (dimethylamino)ethylamino)- 7-methylbenzo[g]pteridine- 2,4(3H,10H)-dione |
| 5 | 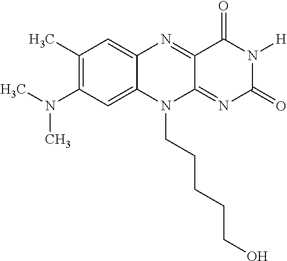 | 358.3 | 5.4 | Method A | Prepared using the synthesis of Example 10 | 8-(dimethylamino)-10-(5- hydroxypentyl)-7- methylbenzo[g]pteridine- 2,4(3H,10H)-dione |
| 6 | 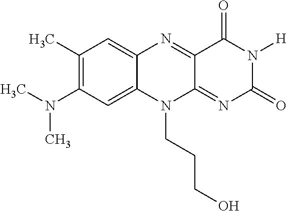 | 330.2 | 5.1 | Method A | Prepared using the synthesis of Example 10 | 8-(dimethylamino)-10-(3- hydroxypropyl)-7- methylbenzo[g]pteridine- 2,4(3H,10H)-dione |
| 7 | 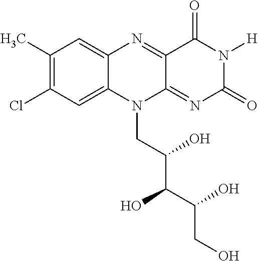 | 397.1 | 4.9 | Method A | Intermediate in the synthesis of Example 4 | 8-chloro-7-methyl-10- ((2S,3S,4R)-2,3,4,5- tetrahydroxypentyl)benzo[g] pteridine-2,4(3H,10H)-dione |
| 8 | 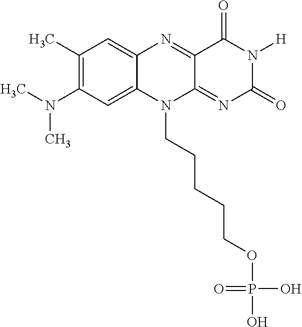 | 436.1 (M − 1) | 5.3 | Method A | Prepared using the synthesis of Example 3 | 5-(8-(dimethylamino)-7- methyl-2,4-dioxo-3,4- dihydrobenzo[g]pteridin- 10(2H)-yl)pentyl dihydrogen phosphate |
| 9 | 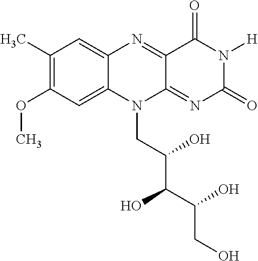 | 390.9 | 4.1 | Method B | Prepared using the synthesis of Example 4 | 8-methoxy-7-methyl-10- ((2S,3S,4R)-2,3,4,5- tetrahydroxypentyl)benzo[g] pteridine-2,4(3H,10H)-dione |
| 10 | 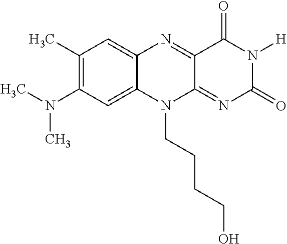 | 344.3 | 4.8 | Method B | Prepared using the synthesis of Example 10 | 8-(dimethylamino)-10-(4- hydroxybutyl)-7- methylbenzo[g]pteridine- 2,4(3H,10H)-dione |
| 11 | 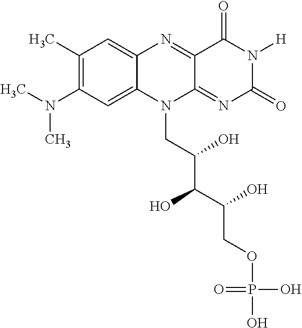 | 484.1 (M − H) | 3.7 | Method B | Prepared using the synthesis of Example 2 | (2R,3S,4S)-5-(8- (dimethylamino)-7-methyl- 2,4-dioxo-3,4- dihydrobenzo[g]pteridin- 10(2H)-yl)-2,3,4- trihydroxypentyl dihydrogen phosphate |
| 12 | 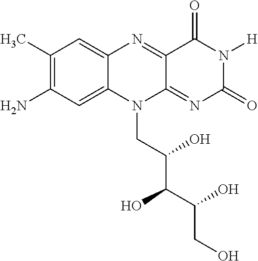 | 378.2 | 4.2 | Method A | Prepared using the synthesis of Example 5 | 8-amino-7-methyl-10- ((2S,3S,4R)-2,3,4,5- tetrahydroxypentyl)benzo[g] pteridine-2,4(3H,10H)-dione |
| 13 | 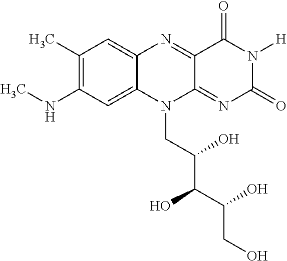 | 392.1 | 1.9 | Method B | Prepared using the synthesis of Example 4 | 7-methyl-8-(methylamino)- 10-((2S,3S,4R)-2,3,4,5- tetrahydroxypentyl)benzo[g] pteridine-2,4(3H,10H)-dione |
| 14 | 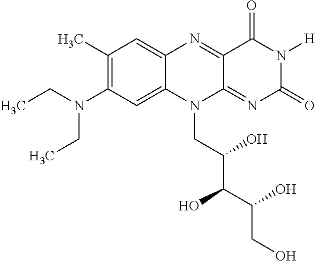 | 434.2 | 2.3 | Method B | Prepared using the synthesis of Example 4 | 8-(diethylamino)-7-methyl- 10-((2S,3S,4R)-2,3,4,5- tetrahydroxypentyl)benzo[g] pteridine-2,4(3H,10H)-dione |
| 15 | 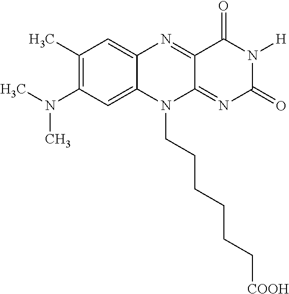 | 398.1 | 3.4 | Method B | Prepared using the synthesis of Example 8 | 7-(8-(dimethylamino)-7- methyl-2,4-dioxo-3,4- dihydrobenzo[g]pteridin- 10(2H)-yl)heptanoic acid |
| 16 | 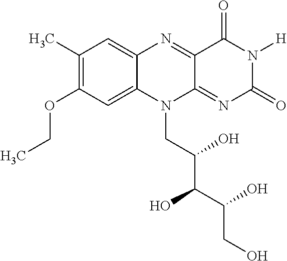 | 407.2 | 2.1 | Method C | Prepared using the synthesis of Example 4 | 8-ethoxy-7-methyl-10- ((2S,3S,4R)-2,3,4,5- tetrahydroxypentyl)benzo[g] pteridine-2,4(3H,10H)-dione |
| 17 | 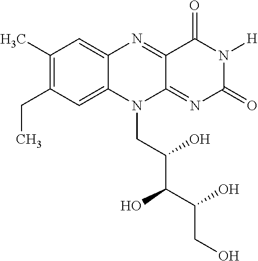 | 391.2 | 2.4 | Method B | Prepared using the synthesis of Example 4 using 3-Ethyl- 4- Methylaniline hydrochloride as starting material | 8-ethyl-7-methyl-10- ((2S,3S,4R)-2,3,4,5- tetrahydroxypentyl)benzo[g] pteridine-2,4(3H,10H)-dione |
| 18 | 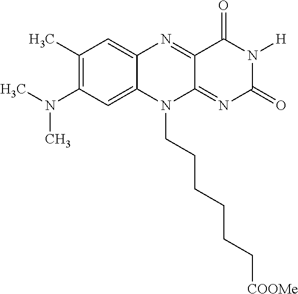 | 414.3 | 3.8 | Method B | Prepared using the synthesis of Example 9 | methyl 7-(8- (dimethylamino)-7-methyl- 2,4-dioxo-3,4- dihydrobenzo[g]pteridin- 10(2H)-yl)heptanoate |
| 19 | 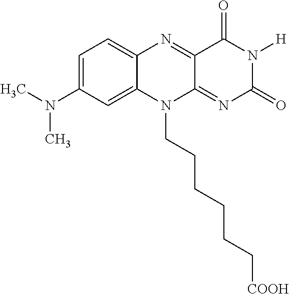 | 384.1 (M − H) | 3 | Method B | Prepared using the synthesis of Example 7 | 7-(8-(dimethylamino)-2,4- dioxo-3,4- dihydrobenzo[g]pteridin- 10(2H)-yl)heptanoic acid |
| 20 | 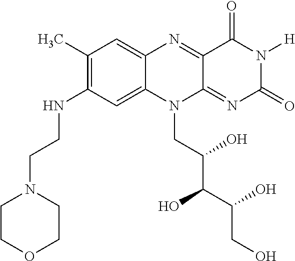 | 491.3 | 1.5 | Method B | Prepared using the synthesis of Example 4 | 7-methyl-8-(2- morpholinoethylamino)-10- ((2S,3S,4R)-2,3,4,5- tetrahydroxypentyl)benzo[g] pteridine-2,4(3H,10H)-dione |
| 21 | 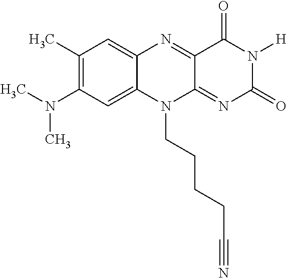 | 353.3 | 3 | Method B | Prepared using the synthesis of Example 11 | 5-(8-(dimethylamino)-7- methyl-2,4-dioxo-3,4- dihydrobenzo[g]pteridin- 10(2H)-yl)pentanenitrile |
| 22 | 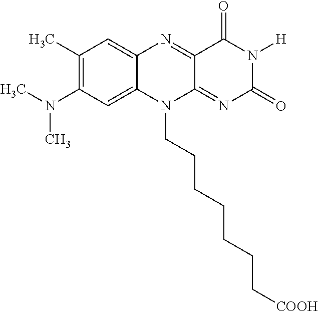 | 414.3 | 3.6 | Method B | Prepared using the synthesis of Example 8 | 8-(8-(dimethylamino)-7- methyl-2,4-dioxo-3,4- dihydrobenzo[g]pteridin- 10(2H)-yl)octanoic acid |
| 23 | 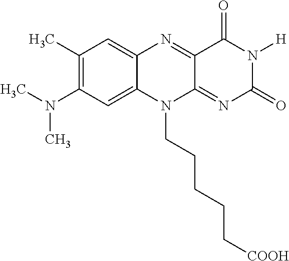 | 386.3 | 3.1 | Method B | Prepared using the synthesis of Example 8 | 6-(8-(dimethylamino)-7- methyl-2,4-dioxo-3,4- dihydrobenzo[g]pteridin- 10(2H)-yl)hexanoic acid |
| 24 | 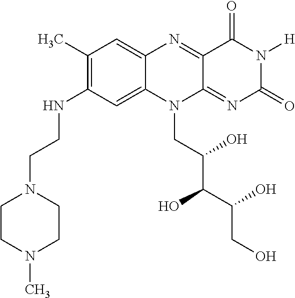 | 504.4 | 1.5 | Method B | Prepared using the synthesis of Example 4 | 7-methyl-8-(2-(4- methylpiperazin-1- yl)ethylamino)-10- ((2S,3S,4R)-2,3,4,5- tetrahydroxypentyl)benzo[g] pteridine-2,4(3H,10H)-dione |
| 25 | 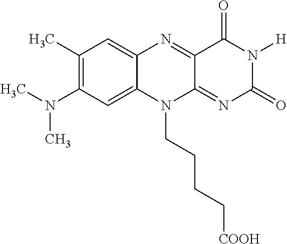 | 372.2 | 2.3 | Method C | Prepared using the synthesis of Example 11 | 5-(8-(dimethylamino)-7- methyl-2,4-dioxo-3,4- dihydrobenzo[g]pteridin- 10(2H)-yl)pentanoic acid |
| 26 | 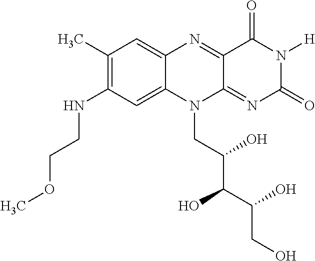 | 436.3 | 4.3 | Method A | Prepared using the synthesis of Example 4 | 8-(2-methoxyethylamino)-7- methyl-10-((2S,3S,4R)- 2,3,4,5- tetrahydroxypentyl)benzo[g] pteridine-2,4(3H,10H)-dione |
| 27 | 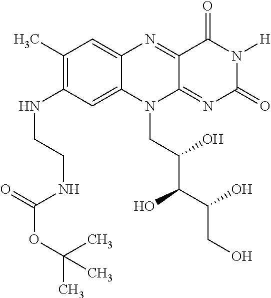 | 521.3 | 5.2 | Method A | Prepared using the synthesis of Example 4 | tert-butyl 2-(7-methyl-2,4- dioxo-10-((2S,3S,4R)- 2,3,4,5-tetrahydroxypentyl)- 2,3,4,10- tetrahydrobenzo[g]pteridin- 8-ylamino)ethylcarbamate |
| 28 | 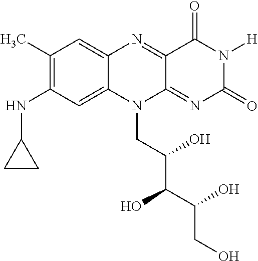 | 418.3 | 4.9 | Method A | Prepared using the synthesis of Example 4 | 8-(cyclopropylamino)-7- methyl-10-((2S,3S,4R)- 2,3,4,5- tetrahydroxypentyl)benzo[g] pteridine-2,4(3H,10H)-dione |
| 29 | 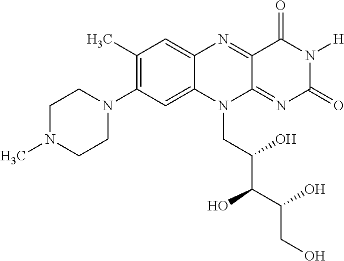 | 461.3 | 3.9 | Method A | Prepared using the synthesis of Example 4 | 7-methyl-8-(4- methylpiperazin-1-yl)-10- ((2S,3S,4R)-2,3,4,5- tetrahydroxypentyl)benzo[g] pteridine-2,4(3H,10H)-dione |
| 30 | 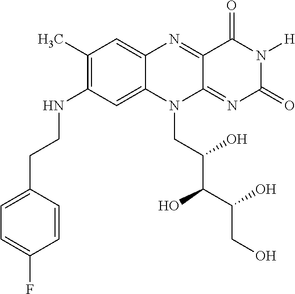 | 500.3 | 5.5 | Method A | Prepared using the synthesis of Example 4 | 8-(4-fluorophenethylamino)- 7-methyl-10-((2S,3S,4R)- 2,3,4,5- tetrahydroxypentyl)benzo[g] pteridine-2,4(3H,10H)-dione |
| 31 | 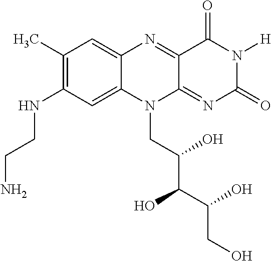 | 421.3 | 3.7 | Method A | Prepared using the synthesis of Example 4 and step 2 of Example 17 | 8-(2-aminoethylamino)-7- methyl-10-((2S,3S,4R)- 2,3,4,5- tetrahydroxypentyl)benzo[g] pteridine-2,4(3H,10H)-dione |
| 32 | 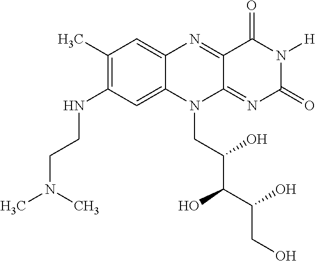 | 449.3 | 3.7 | Method A | Prepared using the synthesis of Example 4 | 8-(2- (dimethylamino)ethylamino)- 7-methyl-10-((2S,3S,4R)- 2,3,4,5- tetrahydroxypentyl)benzo[g] pteridine-2,4(3H,10H)dione |
| 33 | 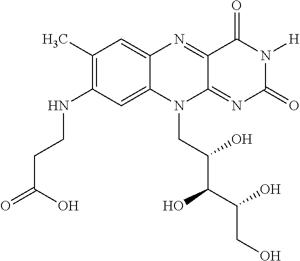 | 450.3 | 4.2 | Method A | Prepared using the synthesis of Example 4 and step 2 of Example 17 | 3-(7-methyl-2,4-dioxo-10- ((2S,3S,4R)-2,3,4,5- tetrahydroxypentyl)- 2,3,4,10- tetrahydrobenzo[g]pteridin- 8-ylamino)propanoic acid |
| 34 | 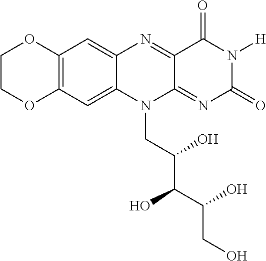 | 407.3 | 4.1 | Method A | Prepared using the synthesis of Example 6 | 12-(2S,3S,4R,5- tetrahydroxy-pentyl)-8,9- dihydro-12H-7,10-dioxa- 1,3,5,12-tetraaza- napthacene-2,4-dione |
| 35 | 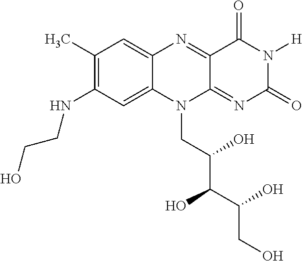 | 422.3 | 4 | Method A | Prepared using the synthesis of Example 4 | 8-(2-hydroxyethylamino)-7- methyl-10-((2S,3S,4R)- 2,3,4,5- tetrahydroxypentyl)benzo[g] pteridine-2,4(3H,10H)-dione |
| 36 | 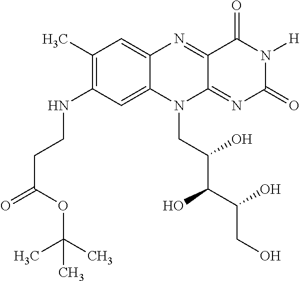 | 506.2 | 5.4 | Method A | Prepared using the synthesis of Example 4 | tert-butyl 3-(7-methyl-2,4- dioxo-10-((2S,3S,4R)- 2,3,4,5-tetrahydroxypentyl)- 2,3,4,10- tetrahydrobenzo[g]pteridin- 8-ylamino)propanoate |
| 37 | 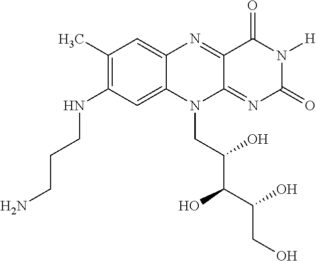 | 435.3 | 3.8 | Method A | Prepared using the synthesis of Example 4 | 8-(3-aminopropylamino)-7- methyl-10-((2S,3S,4R)- 2,3,4,5- tetrahydroxypentyl)benzo[g] pteridine-2,4(3H,10H)-dione |
| 38 | 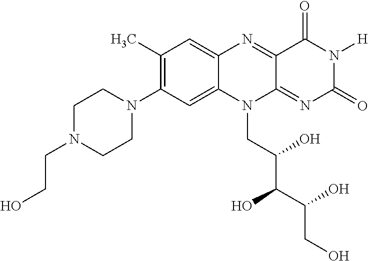 | 491.4 | 3.8 | Method A | Prepared using the synthesis of Example 4 | 8-(4-(2- hydroxyethyl)piperazin-1- yl)-7-methyl-10- ((2S,3S,4R)-2,3,4,5- tetrahydroxypentyl)benzo[g] pteridine-2,4(3H,10H)-dione |
| 39 | 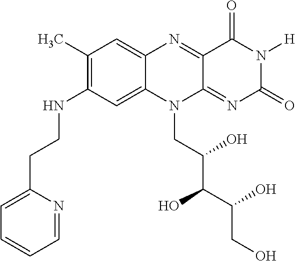 | 483.3 | 4.1 | Method A | Prepared using the synthesis of Example 4 | 7-methyl-8-(2-(pyridin-2- yl)ethylamino)-10- ((2S,3S,4R)-2,3,4,5- tetrahydroxypentyl)benzo[g] pteridine-2,4(3H,10H)-dione |
| 40 | 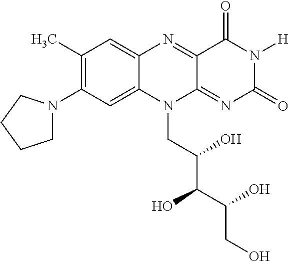 | 432.3 | 4.7 | Method A | Prepared using the synthesis of Example 4 | 7-methyl-8-(pyrrolidin-1- yl)-10-((2S,3S,4R)-2,3,4,5- tetrahydroxypentyl)benzo[g] pteridine-2,4(3H,10H)-dione |
| 41 | 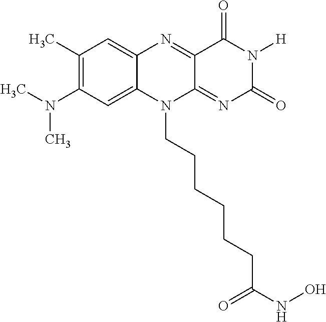 | 413.2 (M − H) | 5.1 | Method A | Prepared using the synthesis of Example 18 | 7-(8-(dimethylamino)-7- methyl-2,4-dioxo-3,4- dihydrobenzo[g]pteridin- 10(2H)-yl)-N- hydroxyheptanamide |
| 42 | 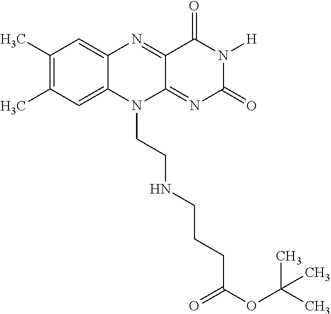 | 428.2 | 5.6 | Method A | Prepared using the synthesis of Reductive Amination of Example 20 | tert-butyl 4-(2-(7,8- dimethyl-2,4-dioxo-3,4- dihydrobenzo[g]pteridin- 10(2H)- yl)ethylamino)butanoate |
| 43 | 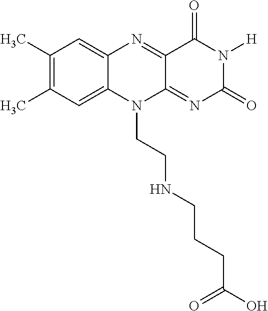 | 372.2 | 4.4 | Method A | Prepared using the synthesis of step 2, Example 15 | 4-(2-(7,8-dimethyl-2,4- dioxo-3,4- dihydrobenzo[g]pteridin- 10(2H)- yl)ethylamino)butanoic acid |
| 44 | 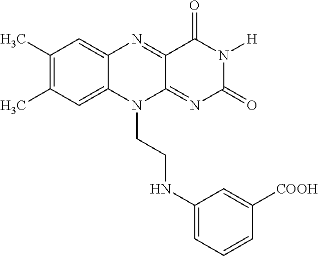 | 404. 1 (M − H) | 6.2 | Method A | Prepared using the syntheses of Example 15, step 2 and Example 17, step 2. | 3-(2-(7,8-dimethyl-2,4- dioxo-3,4- dihydrobenzo[g]pteridin- 10(2H)- yl)ethylamino)benzoic acid |
| 45 | 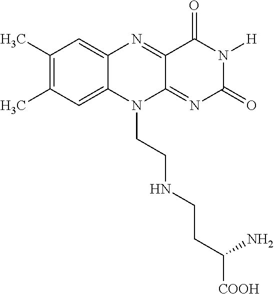 | 385.1 (M − H) | 4 | Method A | Prepared using the method of Example 17 | (S)-2-amino-4-(2-(7,8- dimethyl-2,4-dioxo-3,4- dihydrobenzo[g]pteridin- 10(2H)- yl)ethylamino)butanoic acid |
| 46 | 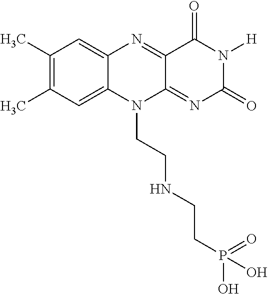 | 394 | 3.9 | Method A | Prepared using the syntheses of Example 19. | 2-(2-(7,8-dimethyl-2,4- dioxo-3,4- dihydrobenzo[g]pteridin- 10(2H)- yl)ethylamino)ethylphos- phonic acid |
| 47 | 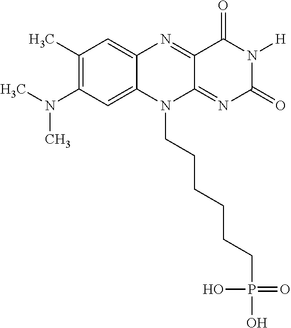 | 436.3 | 9.2 | Method D | Prepared using the synthesis of Example 13 | 6-(8-(dimethylamino)-7- methyl-2,4-dioxo-3,4- dihydrobenzo[g]pteridin- 10(2H)-yl)hexylphosphonic acid |
| 48 | 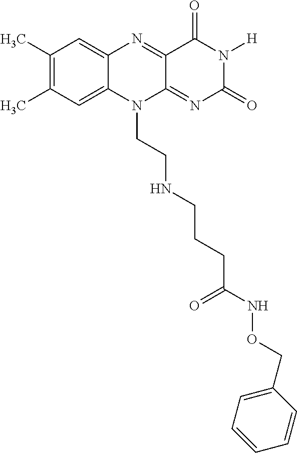 | 477 | 5.8 | Method A | Prepared using the synthesis of Example 22 | N-(benzyloxy)-4-(2-(7,8- dimethyl-2,4-dioxo-3,4- dihydrobenzo[g]pteridin- 10(2H)- yl)ethylamino)butanamide |
| 49 | 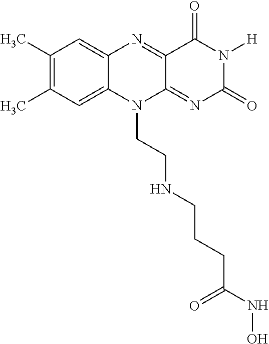 | 387.1 | 4.4 | Method A | Prepared using the synthesis of Example 23 | 4-(2-(7,8-dimethyl-2,4- dioxo-3,4- dihydrobenzo[g]pteridin- 10(2H)-yl)ethylamino)-N- hydroxybutanamide |
| 50 | 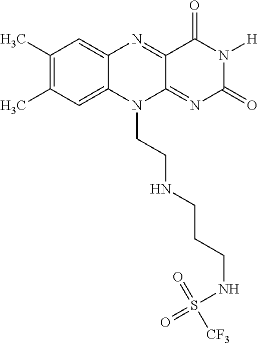 | 475.2 | 6 | Method A | Prepared using the synthesis of Example 15 | N-(3-(2-(7,8-dimethyl-2,4- dioxo-3,4- dihydrobenzo[g]pteridin- 10(2H)- yl)ethylamino)propyl)-1,1,1- trifluoromethanesulfonamide |
| 51 | 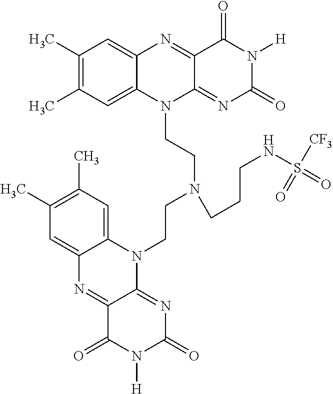 | 743.3 | 6.7 | Method A | Prepared using the synthesis of Example 16 | N-(3-(bis(2-(7,8-dimethyl- 2,4-dioxo-3,4- dihydrobenzo[g]pteridin- 10(2H)- yl)ethyl)amino)propyl)- 1,1,1- trifluoromethanesulfonamide |
| 52 | 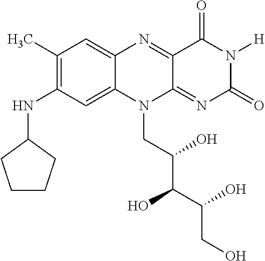 | 446.3 | 5.08 | Method A | Prepared using the synthesis of Example 4 | 8-(cyclopentylamino)-7- methyl-10-((2S,3S,4R)- 2,3,4,5- tetrahydroxypentyl)benzo[g] pteridine-2,4(3H,10H)-dione |
| 53 | 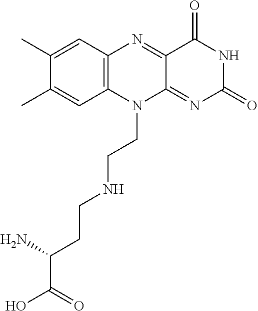 | 387.1 | 4.08 | Method A | Prepared using a procedure similar to that of Example 50 except that the reaction was run at rt for 16 h. | (2R)-2-amino-4-[(2-{7,8- dimethyl-2,4-dioxo- 2H,3H,4H,10H- benzo[g]pteridin-10- yl}ethyl)amino]butanoic acid |
| 54 | 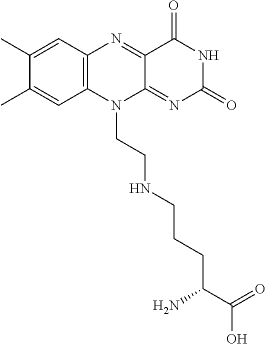 | 401.1 | 4.2 | Method A | Prepared using a procedure similar to that of example 50 except that the reaction was run at rt for 23 h. | (2R)-2-amino-5-[(2-{7,8- dimethyl-2,4-dioxo- 2H,3H,4H,10H- benzo[g]pteridin-10- yl}ethyl)amino]pentanoic acid |
| 55 | 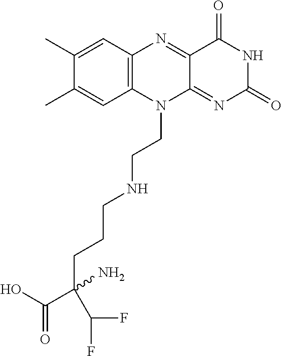 | 451.2 | 4.14 | Method A | Prepared using the procedure of Example 15, Step 2 | 2-amino-2-(difluoromethyl)- 5-[(2-{7,8-dimethyl-2,4- dioxo-2H,3H,4H,10H- benzo[g]pteridin-10- yl}ethyl)amino]pentanoic acid |
| 56 | 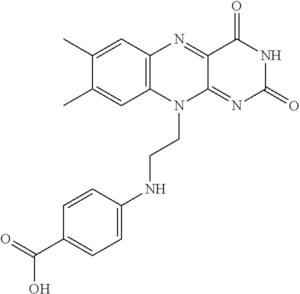 | 404.1 [M − H]− | 2.38 | Method C | Prepared using the procedure of Example 48 | 4-[(2-{7,8-dimethyl-2,4- dioxo-2H,3H,4H,10H- benzo[g]pteridin-10- yl}ethyl)amino]benzoic acid |
| 57 | 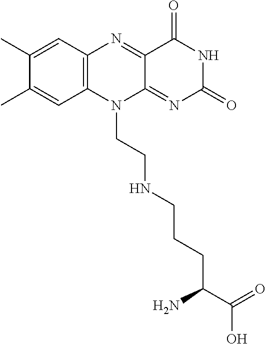 | 401.2 | 4.09 | Method A | Prepared using the procedure of Example 50 | (2S)-2-amino-5-[(2-{7,8- dimethyl-2,4-dioxo- 2H,3H,4H,10H- benzo[g]pteridin-10- yl}ethyl)amino]pentanoic acid |
| 58 | 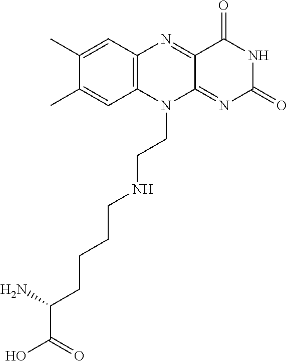 | 415.3 | 4.21 | Method A | Prepared using the procedure of Example 50 | (2R)-2-amino-6-[(2-{7,8- dimethyl-2,4-dioxo- 2H,3H,4H,10H- benzo[g]pteridin-10- yl}ethyl)amino]hexanoic acid |
| 59 | 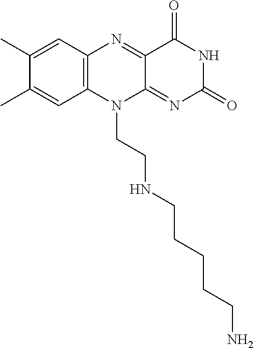 | 371.2 | 4.3 | Method A | Prepared using the procedure of Example 50 | 10-{2-[(5- aminopentyl)amino]ethyl}- 7,8-dimethyl- 2H,3H,4H,10H- benzo[g]pteridine-2,4-dione |
| 60 | 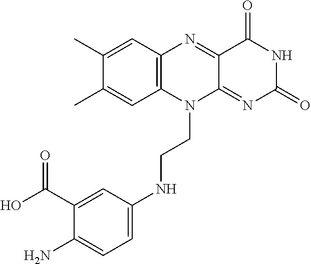 | 419.1 | 4.62 | Method A | Prepared using the procedure of Example 50 | 2-amino-5-[(2-{7,8- dimethyl-2,4-dioxo- 2H,3H,4H,10H- benzo[g]pteridin-10- yl}ethyl)amino]benzoic acid |
| 61 | 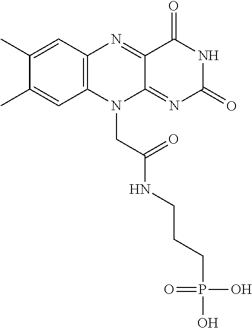 | MH− 420.4 | 4.343 | Method A | Prepared using the procedure of Example 53 | [3-(2-{7,8-dimethyl-2,4- dioxo-2H,3H,4H,10H- benzo[g]pteridin-10- yl}acetamido)propyl]phos- phonic acid |
| 62 | 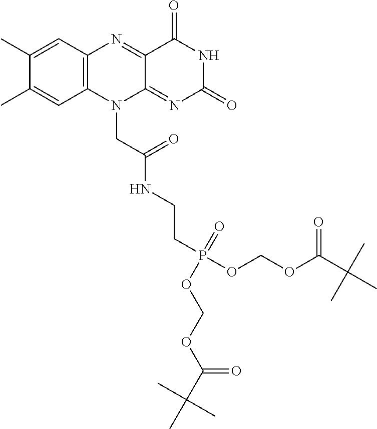 | 636.1 | 7.99 | Method A | Prepared using the procedure of Example 26 | ({[2-(2-{7,8-dimethyl-2,4- dioxo-2H,3H,4H,10H- benzo[g]pteridin-10- yl}acetamido)ethyl]{[(2,2- dimethylpropanoyl)oxy]meth- oxy})phosphoryl}oxy)meth- yl 2,2-dimethylpropanoate |
| 63 | 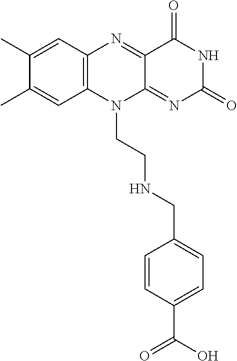 | 418.5 {M − H]− | 5.18 | Method A | Prepared using the procedure of Example 59 | 4-{[(2-{7,8-dimethyl-2,4- dioxo-2H,3H,4H,10H- benzo[g]pteridin-10- yl}ethyl)amino]methyl} benzoic acid |
| 64 | 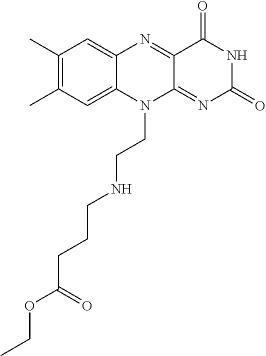 | 400.1 | 5.14 | Method A | Prepared using the procedure of Example 51 | ethyl 4-[(2-{7,8-dimethyl- 2,4-dioxo-2H,3H,4H,10H- benzo[g]pteridin-10- yl}ethyl)amino]butanoate |
| 65 | 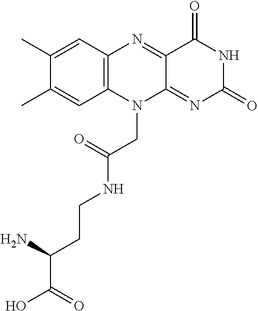 | 399.3 [M− H]− | 4.34 | Method A | Prepared using the procedure of Example 52 | (2S)-2-amino-4-(2-{7,8- dimethyl-2,4-dioxo- 2H,3H,4H,10H- benzo[g]pteridin-10- yl}acetamido)butanoic acid |
| 66 | 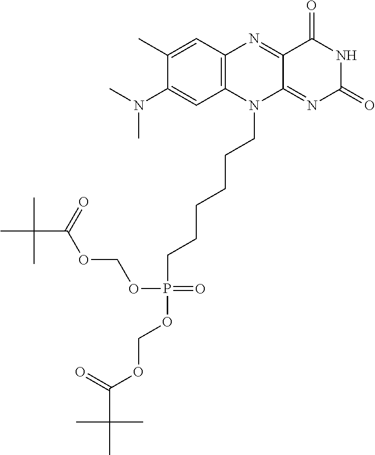 | 664.2 | 5.83 | Method C | Prepared using the procedure of Example 42 | [({6-[8-(dimethylamino)-7- methyl-2,4-dioxo- 2H,3H,4H,10H- benzo[g]pteridin-10- yl]hexyl}({[(2,2- dimethylpropanoyl)oxy]meth- oxy})phosphoryl)oxy]meth- yl 2,2-dimethylpropanoate |
| 67 | 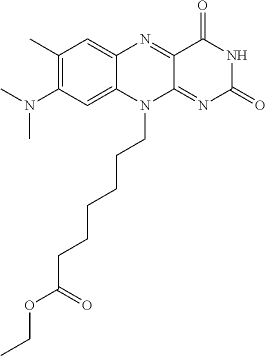 | 428.4 | 6.61 | Method A | Prepared using the procedure of Example 9 | ethyl 7-[8-(dimethylamino)- 7-methyl-2,4-dioxo- 2H,3H,4H,10H- benzo[g]pteridin-10- yl]heptanoate |
| 68 | 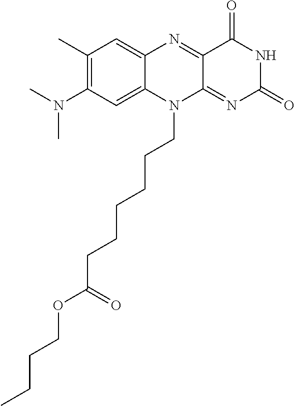 | 456.4 | 7.44 | Method A | Prepared using the procedure of Example 9 | butyl 7-[8-(dimethylamino)- 7-methyl-2,4-dioxo- 2H,3H,4H,10H- benzo[g]pteridin-10- yl]heptanoate |
| 69 | 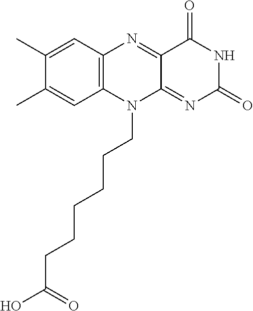 | MH− 396.3 | 6.121 | Method A | Prepared using the procedure of Example 44 | 7-{7,8-dimethyl-2,4-dioxo- 2H,3H,4H,10H- benzo[g]pteridin-10- yl}heptanoic acid |
| 70 | 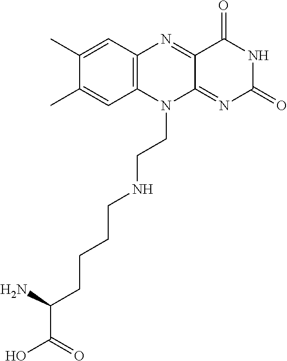 | 415.3 | 4.28 | Method A | Prepared using the procedure of Example 50 | (2S)-2-amino-6-[(2-{7,8- dimethyl-2,4-dioxo- 2H,3H,4H,10H- benzo[g]pteridin-10- yl}ethyl)amino]hexanoic acid |
| 71 | 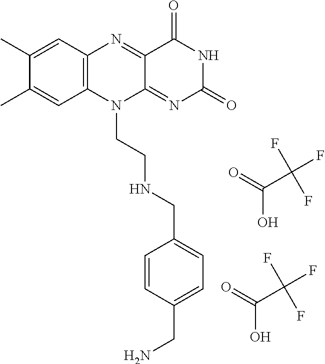 | 405.1 | 4.16 | Method C | Prepared using the procedure of Example 54 | 10-[2-({[4- (aminomethyl)phenyl]methyl} amino)ethyl]-7,8- dimethyl-2H,3H,4H,10H- benzo[g]pteridine-2,4-dione; bis(2,2,2-trifluoroacetic acid) |
| 72 | 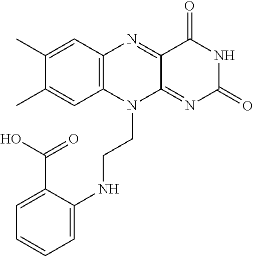 | 404.1 | 6.1 | Method A | Prepared using the procedure of Example 48 | 2-[(2-{7,8-dimethyl-2,4- dioxo-2H,3H,4H,10H- benzo[g]pteridin-10- yl}ethyl)amino]benzoic acid |
| 73 | 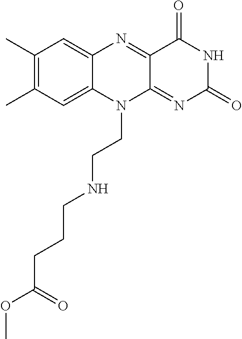 | 386.3 | 5.03 | Method A | Prepared using the procedure of Example 51 | methyl 4-[(2-{7,8-dimethyl- 2,4-dioxo-2H,3H,4H,10H- benzo[g]pteridin-10- yl}ethyl)amino]butanoate |
| 74 | 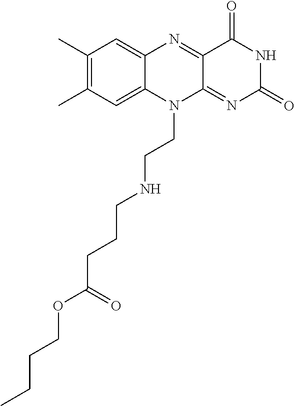 | 428.3 | 5.86 | Method A | Prepared using the procedure of Example 51 | butyl 4-[(2-{7,8-dimethyl- 2,4-dioxo-2H,3H,4H,10H- benzo[g]pteridin-10- yl}ethyl)amino]butanoate |
| 75 | 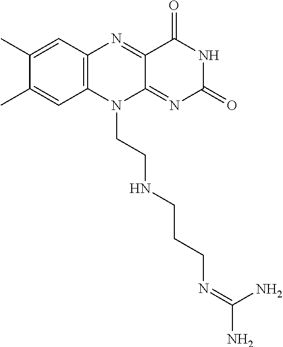 | 385.3 | 4.25 | Method A | Prepared using the procedure of Example 58 | 2-{3-[(2-{7,8-dimethyl-2,4- dioxo-2H,3H,4H,10H- benzo[g]pteridin-10- yl}ethyl)amino]propyl} guanidine |
| 76 | 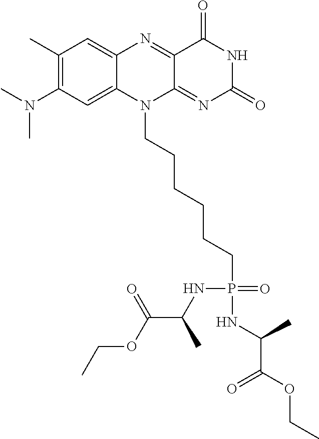 | 634.4 | 3.31 | Method C | Prepared using the procedure of Example 43 | ethyl (2S)-2-[({6-[8- (dimethylamino)-7-methyl- 2,4-dioxo-2H,3H,4H,10H- benzo[g]pteridin-10- yl]hexyl}({[(2S)-1-ethoxy- 1-oxopropan-2- yl]amino})phosphoryl) amino]propanoate |
| 77 | 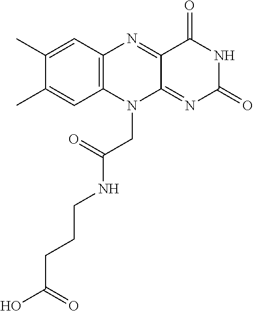 | 386.1 | 1.8 | Method C | Prepared using the procedure of Example 52 | 4-(2-{7,8-dimethyl-2,4- dioxo-2H,3H,4H,10H- benzo[g]pteridin-10- yl}acetamido)butanoic acid |
| 78 | 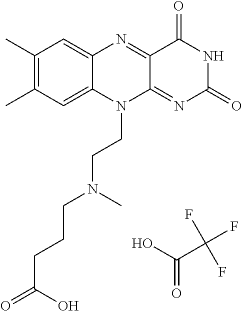 | 386.3 | 4.43 | Method A | Prepared using the procedure of Example 49 | 2,2,2-trifluoroacetic acid; 4- [(2-{7,8-dimethyl-2,4- dioxo-2H,3H,4H,10H- benzo[g]pteridin-10- yl}ethyl)(methyl)amino] butanoic acid |
| 79 | 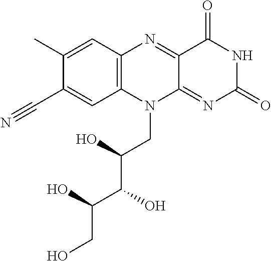 | 410.1 (M + Na)+ | 1.98 | System D | Prepared using the procedure of Example 39 | 7-methyl-2,4-dioxo-10- [(2S,3S,4R)-2,3,4,5- tetrahydroxypentyl]- 2H,3H,4H,10H- benzo[g]pteridine-8- carbonitrile |
| 80 | 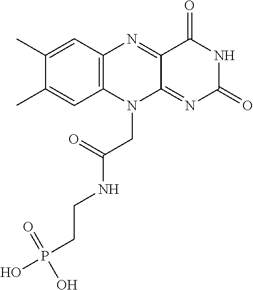 | 406.1 [M − H]− | 3.52 | Method B | Prepared using the procedure of Example 53 | [2-(2-{7,8-dimethyl-2,4- dioxo-2H,3H 4H,10H-, benzo[g]pteridin-10- yl}acetamido)ethyl]phos- phonic acid |
| 81 | 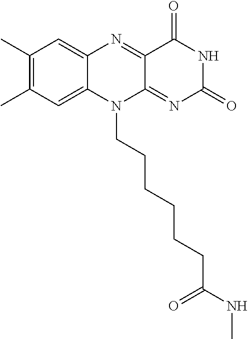 | 384.1 | 2.52 | Method C | Prepared using the procedure of Example 47 | 7-{7,8-dimethyl-2,4-dioxo- 2H,3H,4H,10H- benzo[g]pteridin-10-yl}-N- methylheptanamide |
| 82 | 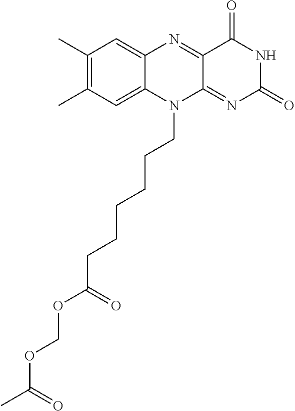 | 443.0 | 4.78 | Method B | Prepared using the procedure of Example 46 | (acetyloxy)methyl 7-{7,8- dimethyl-2,4-dioxo- 2H,3H,4H,10H- benzo[g]pteridin-10- yl}heptanoate |
| 83 | 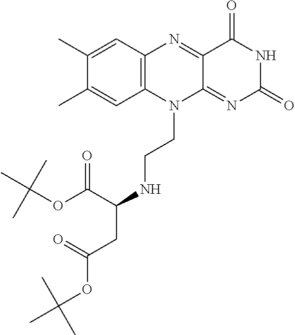 | 514 | 3.02 | Method C | Prepared using the procedure of Example 51 | 1,4-di-tert-butyl (2S)-2-[(2- {7,8-dimethyl-2,4-dioxo- 2H,3H,4H,10H- benzo[g]pteridin-10- yl}ethyl)amino]butanedioate |
| 84 | 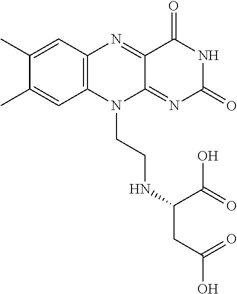 | 402 | 1.43 | Method C | Prepared using the procedure of Example 12 | (2S)-2-[(2-{7,8-dimethyl- 2,4-dioxo-2H,3H,4H,10H- benzo[g]pteridin-10- yl}ethyl)amino]butanedioic acid |
| 85 | 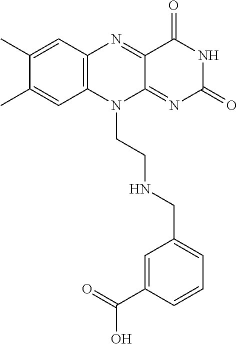 | 418.2 (M − H) | 5.86 | Method C | Prepared using the procedure of Example 48, Step 1 | 3-{[(2-{7,8-dimethyl-2,4- dioxo-2H,3H,4H,10H- benzo[g]pteridin-10- yl}ethyl)amino]methyl} benzoic acid |
| 86 | 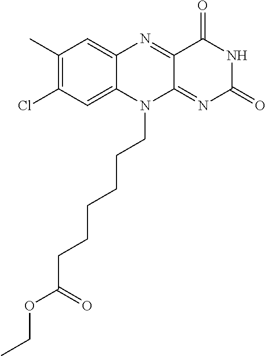 | 419.1 | 5.71 | System B | Prepared using the procedure of Example 28 | ethyl 7-{8-chloro-7-methyl- 2,4-dioxo-2H,3H,4H,10H- benzo[g]pteridin-10- yl}heptanoate |
| 87 | 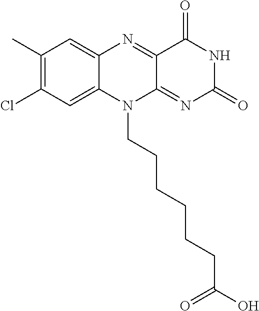 | 391.1 | 4.7 | System B | Prepared using the procedure of Example 29 | 7-{8-chloro-7-methyl-2,4- dioxo-2H,3H,4H,10H- benzo[g]pteridin-10- yl}heptanoic acid |
| 88 | 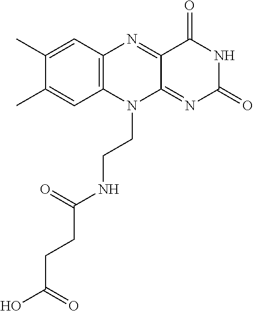 | 386 | 2.01 | Method C | Prepared using the procedure of Example 56 | 3-[(2-{7,8-dimethyl-2,4- dioxo-2H,3H,4H,10H- benzo[g]pteridin-10- yl}ethyl)carbamoyl]propanoic acid |
| 89 | 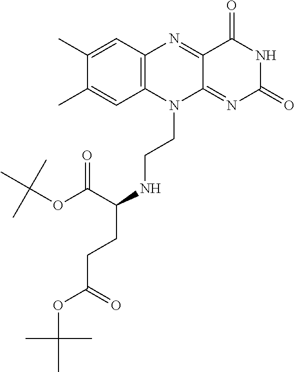 | 528.1 | 3 | Method C | Prepared using the procedure of Example 51 | 1,5-di-tert-butyl (2S)-2-[(2- {7,8-dimethyl-2,4-dioxo- 2H,3H,4H,10H- benzo[g]pteridin-10- yl}ethyl)amino]pentanedioate |
| 90 | 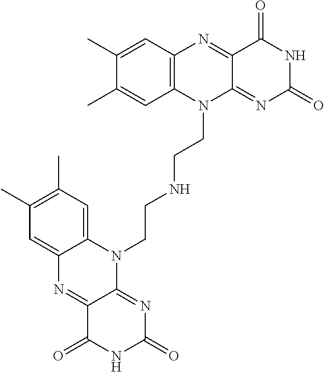 | 554.1 | 3.79 | Method B | Prepared using the procedure of Example 51 | 10-{2-[(2-{7,8-dimethyl- 2,4-dioxo-2H,3H,4H,10H- benzo[g]pteridin-10- yl}ethyl)amino]ethyl}-7,8- dimethyl-2H,3H,4H,10H- benzo[g]pteridine-2,4-dione |
| 91 | 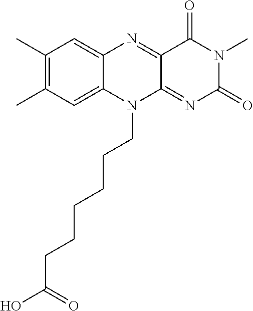 | 385.2 | 2.77 | Method C | Prepared using the procedure of Example 41 | 7-{3,7,8-trimethyl-2,4- dioxo-2H,3H,4H,10H- benzo[g]pteridin-10- yl}heptanoic acid |
| 92 | 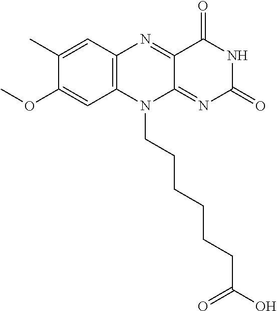 | 387.1 | 4.44 | System B | Prepared using the procedure of Example 30 | 7-{8-methoxy-7-methyl-2,4- dioxo-2H,3H,4H,10H- benzo[g]pteridin-10- yl}heptanoic acid |
| 93 | 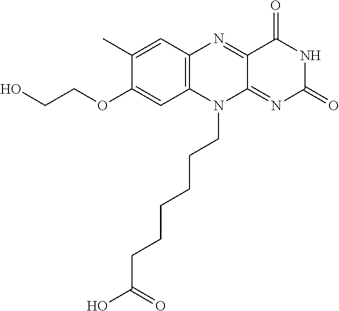 | 417.0 | 4.84 | System A | Prepared using the procedure of Example 32 | 7-[8-(2-hydroxyethoxy)-7- methyl-2,4-dioxo- 2H,3H,4H,10H- benzo[g]pteridin-10- yl]heptanoic acid |
| 94 | 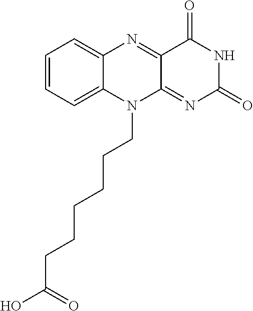 | 343.0 | 2.19 | Method C | Prepared using the procedure of Example 45 | 7-{2,4-dioxo- 2H,3H,4H,10H- benzo[g]pteridin-10- yl}heptanoic acid |
| 95 | 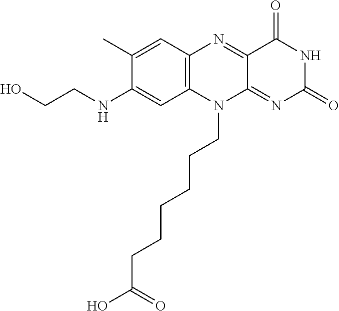 | 416.2 | 4.60 | System A | Prepared using the procedure of Example 31 | 7-{8-[(2- hydroxyethyl)amino]-7- methyl-2,4-dioxo- 2H,3H,4H,10H- benzo[g]pteridin-10- yl}heptanoic acid |
| 96 | 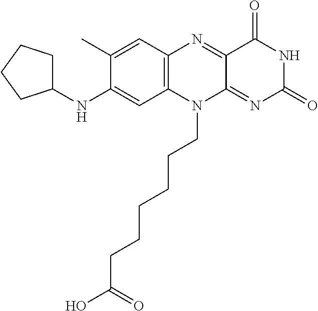 | 440.2 | 6.04 | System A | Prepared using the procedure of Example 33 | 7-[8-(cyclopentylamino)-7- methyl-2,4-dioxo- 2H,3H,4H,10H- benzo[g]pteridin-10- yl]heptanoic acid |
| 97 | 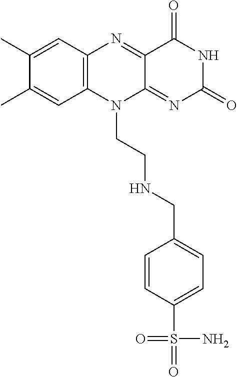 | 455.1 | 1.67 | Method B | Prepared using the procedure of Example 48, Step 1 | 4-{[(2-{7,8-dimethyl-2,4- dioxo-2H,3H,4H,10H- benzo[g]pteridin-10- yl}ethyl)amino]methyl} benzene-1-sulfonamide |
| 98 | 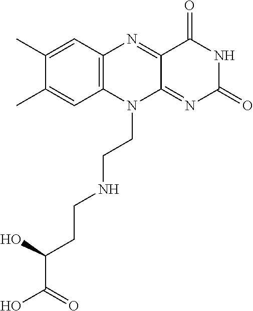 | 388 | 1.47 | Method C | Prepared using the procedure of Example 50 | (2S)-4-[(2-{7,8-dimethyl- 2,4-dioxo-2H,3H,4H,10H- benzo[g]pteridin-10- yl}ethyl)amino]-2- hydroxybutanoic acid |
| 99 | 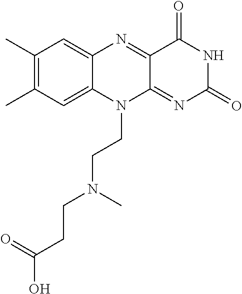 | MH− 370.1 | 4.763 | Method A | Prepared using the procedure of Example 49 | 3-[(2-{7,8-dimethyl-2,4- dioxo-2H,3H,4H,10H- benzo[g]pteridin-10- yl}ethyl)(methyl)amino] propanoic acid |
| 100 | 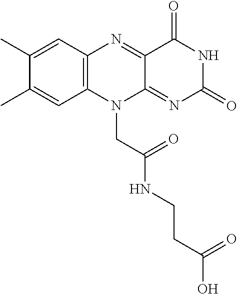 | 371.9 | 6.242 | Method A | Prepared using the procedure of Example 52 | 3-(2-{7,8-dimethyl-2,4- dioxo-2H,3H,4H,10H- benzo[g]pteridin-10- yl}acetamido)propanoic acid |
| 101 | 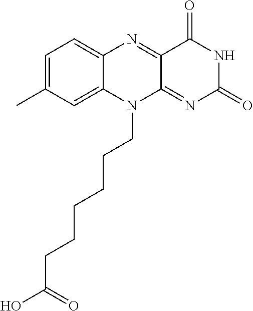 | 357.13 | 6.272 | Method A | Prepared using the procedure of Example 44 | 7-{8-methyl-2,4-dioxo- 2H,3H,4H,10H- benzo[g]pteridin-10- yl}heptanoic acid |
| 102 | 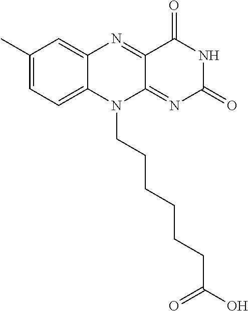 | MH− 355.13 | 6.346 | Method A | Prepared using the procedure of Example 44 | 7-{7-methyl-2,4-dioxo- 2H,3H,4H,10H- benzo[g]pteridin-10- yl}heptanoic acid |
| 103 | 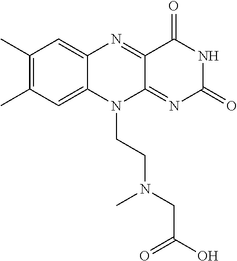 | 358.13 | 5.13 | Method A | Prepared using the procedure of Example 49 | 2-[(2-{7,8-dimethyl-2,4- dioxo-2H,3H,4H,10H- benzo[g]pteridin-10- yl}ethyl)(methyl)amino] acetic acid |
| 104 | 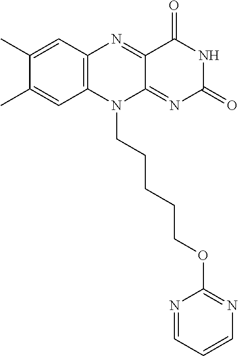 | 407.1 | 2.86 | Method C | Prepared using the procedure of Example 57 | 7,8-dimethyl-10-[5- (pyrimidin-2-yloxy)pentyl]- 2H,3H,4H,10H- benzo[g]pteridine-2,4-dione |
| 105 | 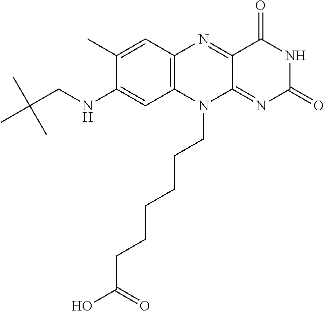 | 442.1 | 3.01 | System C | Prepared using the procedure of Example 34 | 7-{8-[(2,2- dimethylpropyl)amino]-7- methyl-2,4-dioxo- 2H,3H,4H,10H- benzo[g]pteridin-10- yl}heptanoic acid |
| 106 | 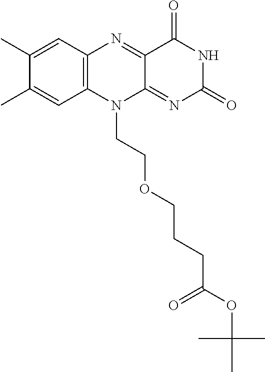 | 429.1 | 3.52 | System D | Prepared using the procedure of Example 37, Step 2 | tert-butyl 4-(2-{7,8- dimethyl-2,4-dioxo- 2H,3H,4H,10H- benzo[g]pteridin-10- yl}ethoxy)butanoate |
| 107 | 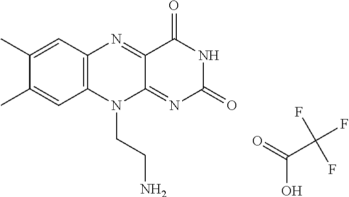 | 286 | 3.24 | Method C | Prepared using the procedure of Example 40 | 10-(2-aminoethyl)-7,8- dimethyl-2H,3H,4H,10H- benzo[g]pteridine-2,4-dione; 2,2,2-trifluoroacetic acid |
| 108 | 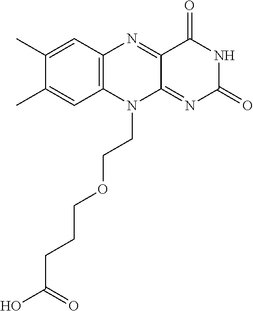 | 373.0 | 2.38 | System D | Prepared using the procedure of Example 37 | 4-(2-{7,8-dimethyl-2,4- dioxo-2H,3H,4H,10H- benzo[g]pteridin-10- yl}ethoxy)butanoic acid |
| 109 | 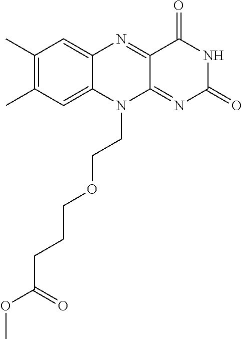 | 387.1 | 2.78 | System D | Prepared using the procedure of Example 38 | methyl 4-(2-{7,8-dimethyl- 2,4-dioxo-2H,3H,4H,10H- benzo[g]pteridin-10- yl}ethoxy)butanoate |
| 110 | 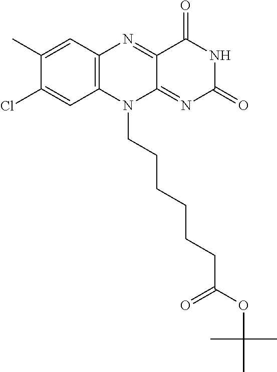 | 447.0 | 4.2 | System D | Prepared using the procedure of Example 36, Step 3 | tert-butyl 7-{8-chloro-7- methyl-2,4-dioxo- 2H,3H,4H,10H- benzo[g]pteridin-10- yl}heptanoate |
| 111 | 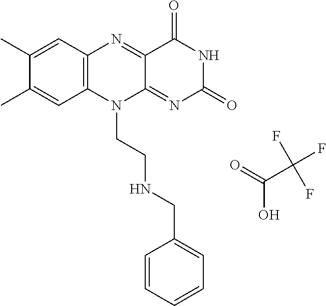 | 376.1 | 2.11 | Method C | Prepared using the procedure of Example 55 | 10-[2-(benzylamino)ethyl]- 7,8-dimethyl- 2H,3H,4H,10H- benzo[g]pteridine-2,4-dione; 2,2,2-trifluoroacetic acid |
| 112 | 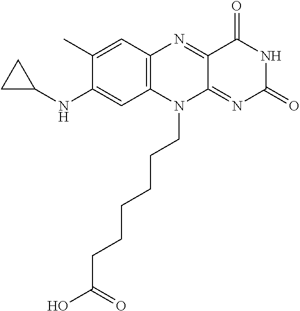 | 412.1 | 2.58 | System D | Prepared using the procedure of Example 35 | 7-[8-(cyclopropylamino)-7- methyl-2,4-dioxo- 2H,3H,4H,10H- benzo[g]pteridin-10- yl]heptanoic acid |
| 113 | 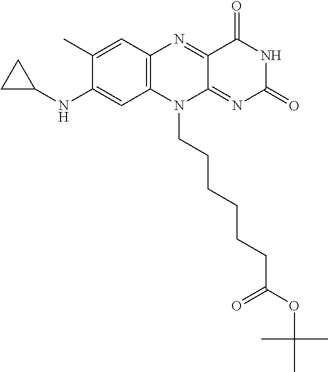 | 468.1 | 3.63 | System C | Prepared using the procedure of Example 36 | tert-butyl 7-[8- (cyclopropylamino)-7- methyl-2,4-dioxo- 2H,3H,4H,10H- benzo[g]pteridin-10- yl]heptanoate |
| 114 | 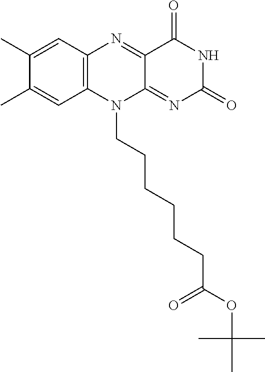 | 427.1 | 3.64 | Method B | Prepared using the procedure of Example 24 | tert-butyl 7-{7,8-dimethyl- 2,4-dioxo-2H,3H,4H,10H- benzo[g]pteridin-10- yl}heptanoate |
| 115 | 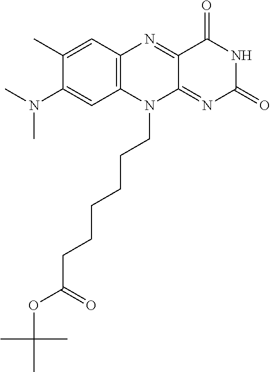 | 456.1 | 3.74 | System C | Prepared using the procedure of Example 36, step 4 | tert-butyl 7-[8- (dimethylamino)-7-methyl- 2,4-dioxo-2H,3H,4H,10H- benzo[g]pteridin-10- yl]heptanoate |
| 116 | 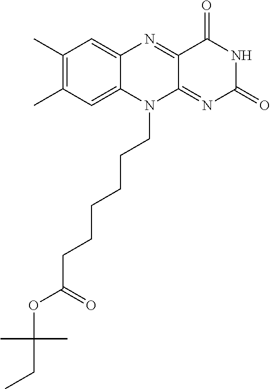 | 440.9 [M + H]+ 463.0 [M + Na]+ | 8.81 | Method A | Prepared using the procedure of Example 66 | 2-methylbutan-2-yl 7-{7,8- dimethyl-2,4-dioxo- 2H,3H,4H,10H- benzo[g]pteridin-10- yl}heptanoate |
| 117 | 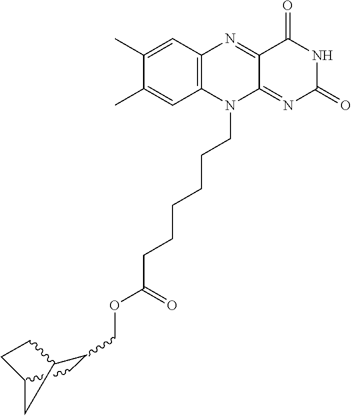 | 479.2 | 5.65 | Method C | Prepared using the procedure of Example 25 | bicyclo[2.2.1]heptan-2- ylmethyl 7-{7,8-dimethyl- 2,4-dioxo-2H,3H,4H,10H- benzo[g]pteridin-10- yl}heptanoate |
| 118 | 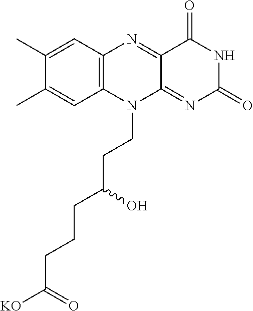 | 424.88 | 3.87 | System B | Prepared using the procedure of Example 64 | potassium 7-{7,8-dimethyl- 2,4-dioxo-2H,3H,4H,10H- benzo[g]pteridin-10-yl}-5- hydroxyheptanoate |
| 119 | 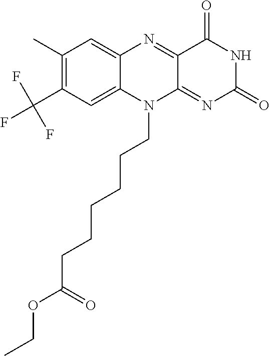 | 451.1 | 5.83 | System B | Prepared using the procedure of Example 63, Step 4 | ethyl 7-[7-methyl-2,4-dioxo- 8-(trifluoromethyl)- 2H,3H,4H,10H- benzo[g]pteridin-10- yl]heptanoate |
| 120 | 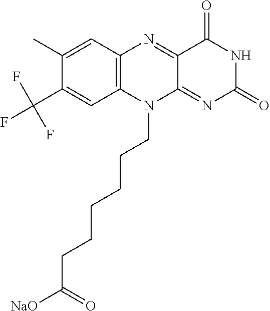 | 423.0 (M − H)− | 4.92 | System B | Prepared using the procedure of Example 63 | sodium 7-(7-methyl-2,4- dioxo-8-(trifluoromethyl)- 3,4- dihydrobenzo[g]pteridin- 10(2H)-yl)heptanoate |
| 121 | 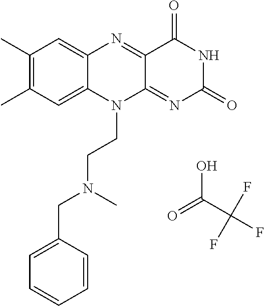 | 389.9 | 3.9 | Method C | Prepared using the procedure of Example 49 | 10-{2- [benzyl(methyl)amino]ethyl}- 7,8-dimethyl- 2H,3H,4H,10H- benzo[g]pteridine-2,4-dione; 2,2,2-trifluoroacetic acid |
| 122 | 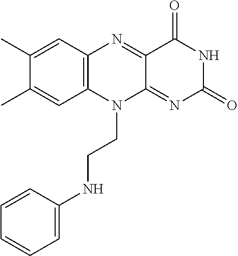 | 361.9 | 4.82 | Method C | Prepared using the procedure of Example 49 | 7,8-dimethyl-10-[2- (phenylamino)ethyl]- 2H,3H,4H,10H- benzo[g]pteridine-2,4-dione |
| 123 | 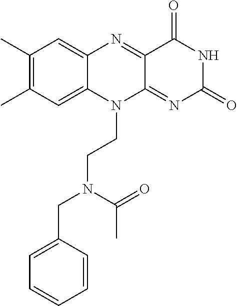 | 417.9 | 4.56 | Method C | Prepared using the procedure of Example 72 | N-benzyl-N-(2-{7,8- dimethyl-2,4-dioxo- 2H,3H,4H,10H- benzo[g]pteridin-10- yl}ethyl)acetamide |
| 124 | 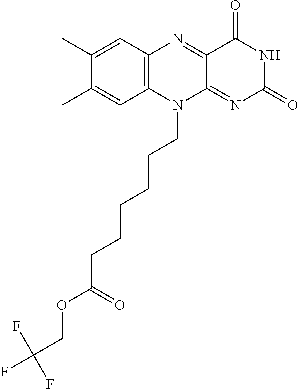 | 453.1 | 5.51 | Method C | Prepared using the procedure of Example 25 | 2,2,2-trifluoroethyl 7-{7,8- dimethyl-2,4-dioxo- 2H,3H,4H,10H- benzo[g]pteridin-10- yl}heptanoate |
| 125 | 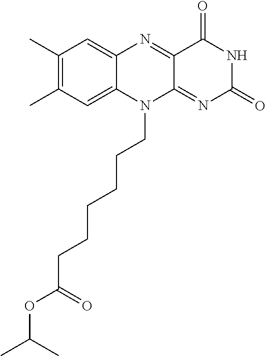 | ESI(+) m/z = 413.1 | | | Prepared using the procedure of Example 25 | propan-2-yl 7-{7,8- dimethyl-2,4-dioxo- 2H,3H,4H,10H- benzo[g]pteridin-10- yl}heptanoate |
| 126 | 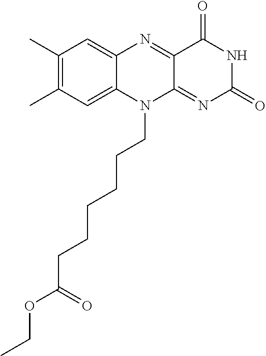 | 399.0 | 5.19 | Method C | Prepared using the procedure of Example 25 | ethyl 7-{7,8-dimethyl-2,4- dioxo-2H,3H,4H,10H- benzo[g]pteridin-10- yl}heptanoate |
| 127 | 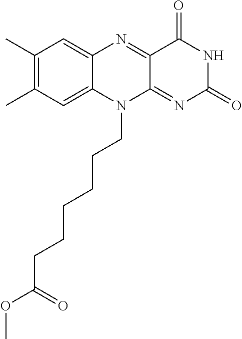 | 385.0 | 4.94 | Method C | Prepared using the procedure of Example 25 | methyl 7-{7,8-dimethyl-2,4- dioxo-2H,3H,4H,10H- benzo[g]pteridin-10- yl}heptanoate |
| 128 | 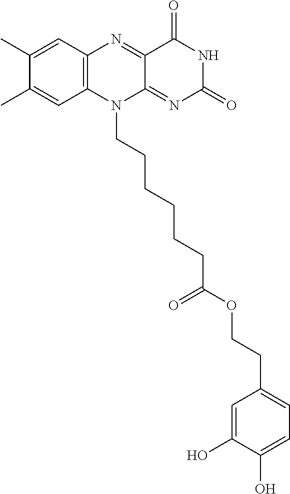 | 507.0 | 5.03 | Method C | Prepared using the procedure of Example 25 | 2-(3,4- dihydroxyphenyl)ethyl 7- {7,8-dimethyl-2,4-dioxo- 2H,3H,4H,10H- benzo[g]pteridin-10- yl}heptanoate |
| 129 | 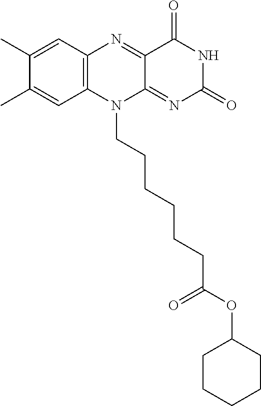 | 453.0 | 6.01 | Method C | Prepared using the procedure of Example 25 | cyclohexyl 7-{7,8-dimethyl- 2,4-dioxo-2H,3H,4H,10H- benzo[g]pteridin-10- yl}heptanoate |
| 130 | 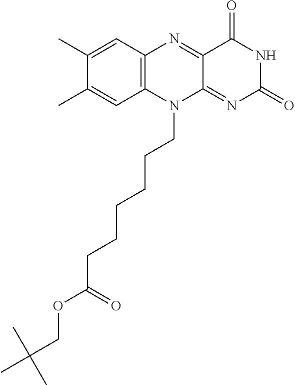 | 441.0 | 5.96 | Method C | Prepared using the procedure of Example 25 | 2,2-dimethylpropyl 7-{7,8- dimethyl-2,4-dioxo- 2H,3H,4H,10H- benzo[g]pteridin-10- yl}heptanoate |
| 131 | 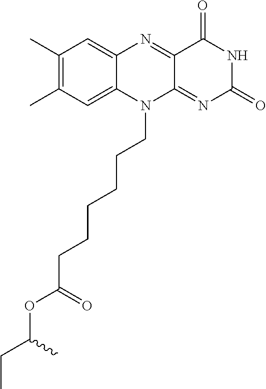 | 426.9 | 5.69 | Method C | Prepared using the procedure of Example 25 | butan-2-yl 7-{7,8-dimethyl- 2,4-dioxo-2H,3H,4H,10H- benzo[g]pteridin-10- yl}heptanoate |
| 132 | 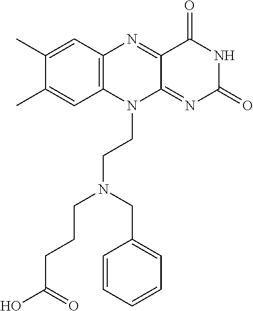 | 461.9 | 3.95 | Method C | Prepared using the procedure of Example 75, Step 2 | 4-[benzyl(2-{7,8-dimethyl- 2,4-dioxo-2H,3H,4H,10H- benzo[g]pteridin-10- yl}ethyl)amino]butanoic acid |
| 133 | 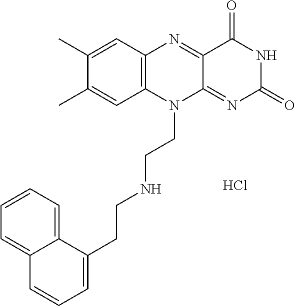 | 440.1 | 4.46 | Method C | Prepared using the procedure of Example 70 | 7,8-dimethyl-10-(2-{[2- (naphthalen-1- yl)ethyl]amino}ethyl)- 2H,3H,4H,10H- benzo[g]pteridine-2,4-dione hydrochloride |
| 134 | 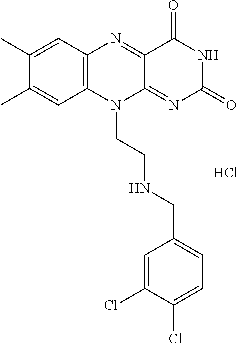 | 443.9 | 4.41 | Method C | Prepared using the procedure of Example 70 | 10-(2-{[(3,4- dichlorophenyl)methyl] amino}ethyl)-7,8-dimethyl- 2H,3H,4H,10H- benzo[g]pteridine-2,4-dione hydrochloride |
| 135 | 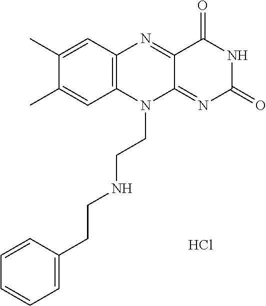 | 390.1 | 4.04 | Method C | Prepared using the procedure of Example 70 | 7,8-dimethyl-10-{2-[(2- phenylethyl)amino]ethyl}- 2H,3H,4H,10H- benzo[g]pteridine-2,4-dione hydrochloride |
| 136 | 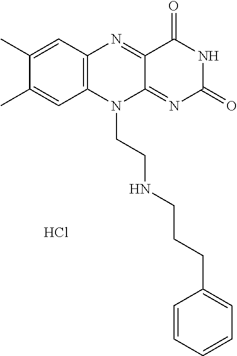 | 404.1 | 4.28 | Method C | Prepared using the procedure of Example 70 | 7,8-dimethyl-10-{2-[(3- phenylpropyl)amino]ethyl}- 2H,3H,4H,10H- benzo[g]pteridine-2,4-dione hydrochloride |
| 137 | 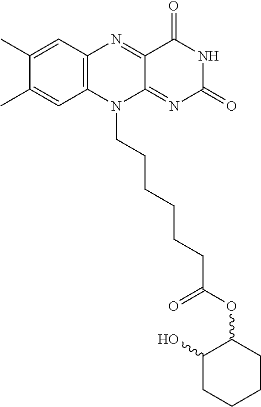 | 469.1 | 5.06 | Method C | Prepared using the procedure of Example 25 | 2-hydroxycyclohexyl 7- {7,8-dimethyl-2,4-dioxo- 2H,3H,4H,10H- benzo[g]pteridin-10- yl}heptanoate |
| 138 | 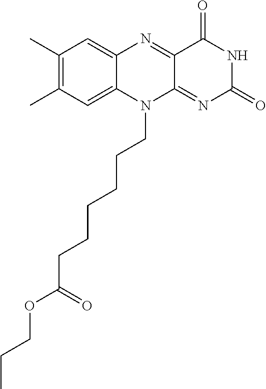 | 413.2 | 5.46 | Method C | Prepared using the procedure of Example 25 | propyl 7-{7,8-dimethyl-2,4- dioxo-2H,3H,4H,10H- benzo[g]pteridin-10- yl}heptanoate |
| 139 | 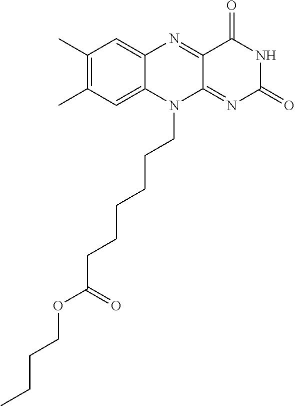 | 427.2 | 5.75 | Method C | Prepared using the procedure of Example 25 | butyl 7-{7,8-dimethyl-2,4- dioxo-2H,3H,4H,10H- benzo[g]pteridin-10- yl}heptanoate |
| 140 | 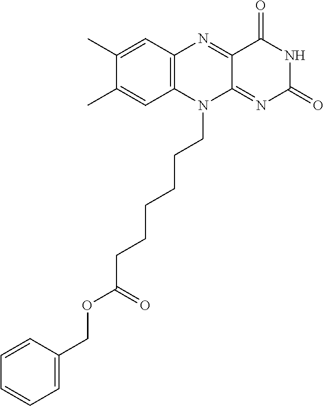 | 461.2 | 5.72 | Method C | Prepared using the procedure of Example 24, Step 2 | benzyl 7-{7,8-dimethyl-2,4- dioxo-2H,3H,4H,10H- benzo[g]pteridin-10- yl}heptanoate |
| 141 | 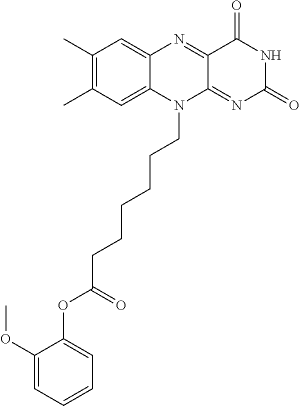 | 477.1 | 5.58 | Method C | Prepared using the procedure of Example 24, Step 2 | 2-methoxyphenyl 7-{7,8- dimethyl-2,4-dioxo- 2H,3H,4H,10H- benzo[g]pteridin-10- yl}heptanoate |
| 142 | 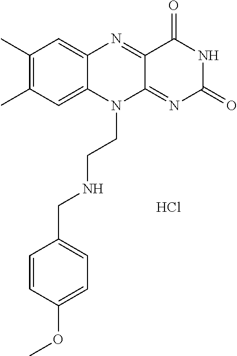 | 406 | 3.93 | Method C | Prepared using the procedure of Example 70 | 10-(2-{[(4- methoxyphenyl)methyl] amino}ethyl)-7,8-dimethyl- 2H,3H,4H,10H- benzo[g]pteridine-2,4-dione hydrochloride |
| 143 | 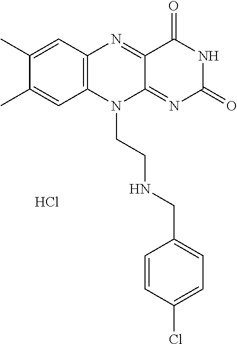 | 410.1 | 4.17 | Method C | Prepared using the procedure of Example 70 | 10-(2-{[(4- chlorophenyl)methyl]amino}- ethyl)-7,8-dimethyl- 2H,3H,4H,10H- benzo[g]pteridine-2,4-dione hydrochloride |
| 144 | 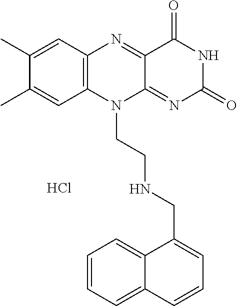 | 426 | 4.11 | Method C | Prepared using the procedure of Example 70 | 7,8-dimethyl-10-{2- [(naphthalen-1- ylmethyl)amino]ethyl}- 2H,3H,4H,10H- benzo[g]pteridine-2,4-dione hydrochloride |
| 145 | 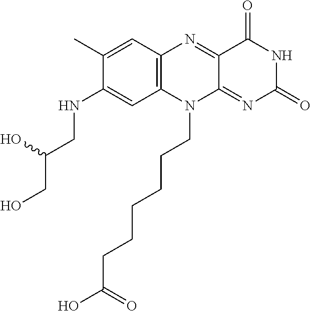 | 446.2 | 2.01 | Method C | Prepared using the procedure of Example 79 | 7-{8-[(2,3- dihydroxypropyl)amino]-7- methyl-2,4-dioxo- 2H,3H,4H,10H- benzo[g]pteridin-10- yl}heptanoic acid |
| 146 | 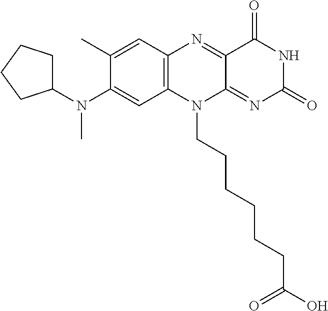 | 454.3 | 3.26 | Method C | Prepared using the procedure of Example 80 | 7-{8- [cyclopentyl(methyl)amino]- 7-methyl-2,4-dioxo- 2H,3H,4H,10H- benzo[g]pteridin-10- yl}heptanoic acid |
| 147 | 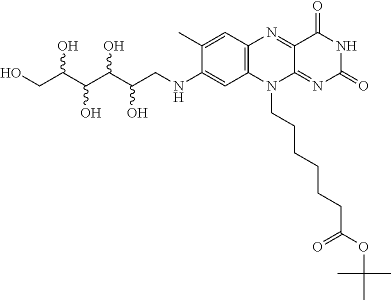 | 592.1 | 2.56 | Method C | Prepared using the procedure of Example 81 | tert-butyl 7-{7-methyl-2,4- dioxo-8-[(2,3 ,4,5,6- pentahydroxyhexyl)amino]- 2H,3H,4H,10H- benzo[g]pteridin-10- yl}heptanoate |
| 148 | 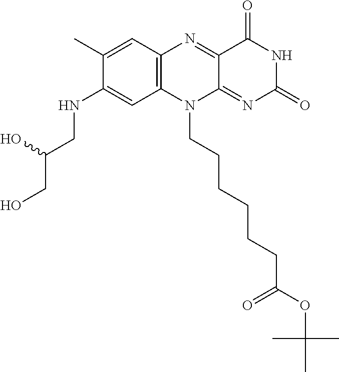 | 502 | 2.77 | Method C | Prepared using the procedure of Example 79 | tert-butyl 7-{8-[(2,3- dihydroxypropyl)amino]-7- methyl-2,4-dioxo- 2H,3H,4H,10H- benzo[g]pteridin-10- yl}heptanoate |
| 149 | 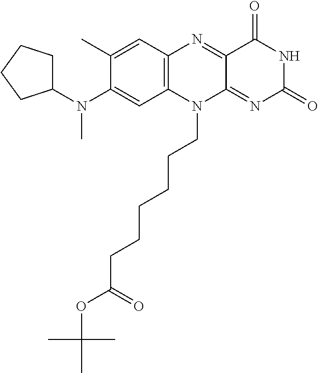 | 510.2 | 4.28 | Method C | Prepared using the procedure of Example 80 | tert-butyl 7-{8- [cyclopentyl(methyl)amino]- 7-methyl-2,4-dioxo- 2H,3H,4H,10H- benzo[g]pteridin-10- yl}heptanoate |
| 150 | 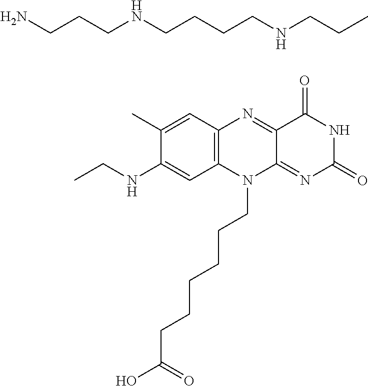 | 557.3 | 3.27 | Method C | Prepared using the procedure of Example 82 | 7-(8-{[3-({4-[(3- aminopropyl)amino]butyl} amino)propyl]amino}-7- methyl-2,4-dioxo- 2H,3H,4H,10H- benzo[g]pteridin-10- yl)heptanoic acid |
| 151 | 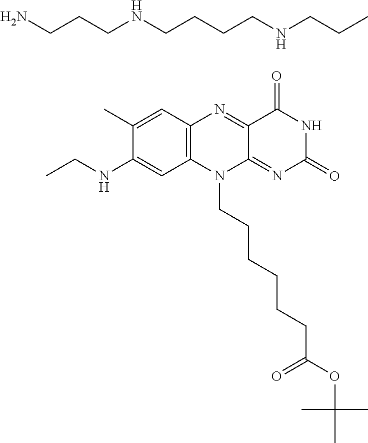 | 613.3 | 2.26 | Method C | Prepared using the procedure of Example 82 | tert-butyl 7-(8-{[3-({4-[(3- aminopropyl)amino]butyl} amino)propyl]amino}-7- methyl-2,4-dioxo- 2H,3H,4H,10H- benzo[g]pteridin-10- yl)heptanoate |
| 152 | 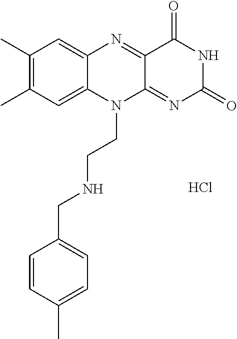 | 390.2 | 2.58 | Method C | Prepared using the procedure of Example 70 | 7,8-dimethyl-10-(2-{[(4- methylphenyl)methyl]amino} ethyl)-2H,3H,4H,10H- benzo[g]pteridine-2,4-dione hydrochloride |
| 153 | 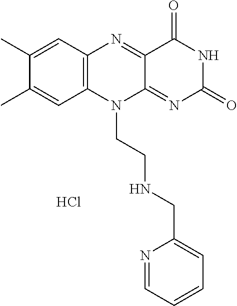 | 377.1 | 3.57 | Method C | Prepared using the procedure of Example 70 | 7,8-dimethyl-10-{2- [(pyridin-2- ylmethyl)amino]ethyl}- 2H,3H,4H,10H- benzo[g]pteridine-2,4-dione hydrochloride |
| 154 | 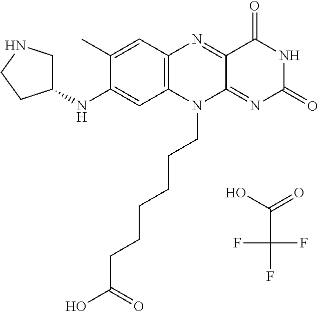 | 441.2 | 1.99 | Method C | Prepared using the procedure of Example 61 | 2,2,2-trifluoroacetic acid; 7- {7-methyl-2,4-dioxo-8- [(3R)-pyrrolidin-3- ylamino]-2H,3H,4H,10H- benzo[g]pteridin-10- yl}heptanoic acid |
| 155 | 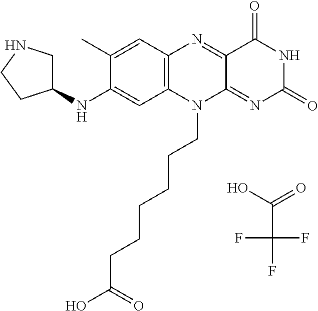 | 441.2 | 1.99 | System C | Prepared using the procedure of Example 62 | 2,2,2-trifluoroacetic acid; 7- {7-methyl-2,4-dioxo-8- [(3S)-pyrrolidin-3-ylamino]- 2H,3H,4H,10H- benzo[g]pteridin-10- yl}heptanoic acid |
| 156 | 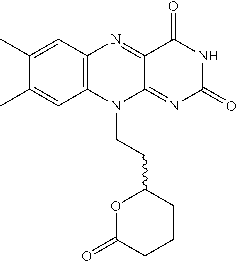 | 367.0 | 4.14 | System B | Prepared using the procedure of Example 64, Step 3 | 7,8-dimethyl-10-[2-(6- oxooxan-2-yl)ethyl]- 2H,3H,4H,10H- benzo[g]pteridine-2,4-dione |
| 157 | 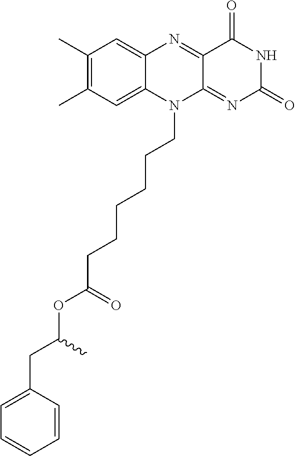 | 489.1 | 5.41 | Method C | Prepared using the procedure of Example 25 | 1-phenylpropan-2-yl 7-{7,8- dimethyl-2,4-dioxo- 2H,3H,4H,10H- benzo[g]pteridin-10- yl}heptanoate |
| 158 | 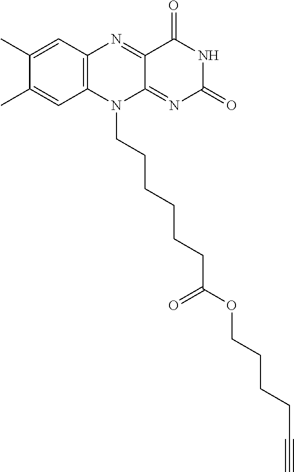 | 451.3 | 5.02 | Method C | Prepared using the procedure of Example 25 | hex-5-yn-1-yl 7-{7,8- dimethyl-2,4-dioxo- 2H,3H,4H,10H- benzo[g]pteridin-10- yl}heptanoate |
| 159 | 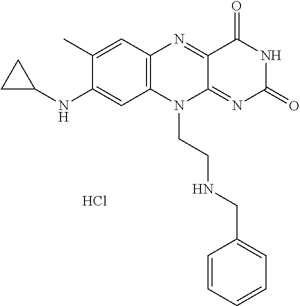 | 417 | 3.51 | Method C | Prepared using the procedure of Example 73 | 10-[2-(benzylamino)ethyl]- 8-(cyclopropylamino)-7- methyl-2H,3H,4H,10H- benzo[g]pteridine-2,4-dione hydrochloride |
| 160 | 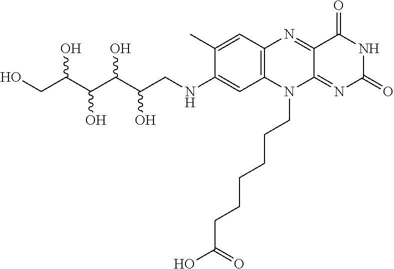 | 536.2 | 3.27 | Method C | Prepared using the procedure of Example 81 | 7-{7-methyl-2,4-dioxo-8- [(2,3,4,5,6- pentahydroxyhexyl)amino]- 2H,3H,4H,10H- benzo[g]pteridin-10- yl}heptanoic acid |
| 161 | 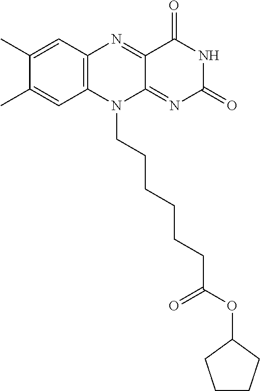 | 439.1 | 5.09 | Method C | Prepared using the procedure of Example 25 | cyclopentyl 7-{7,8- dimethyl-2,4-dioxo- 2H,3H,4H,10H- benzo[g]pteridin-10- yl}heptanoate |
| 162 | 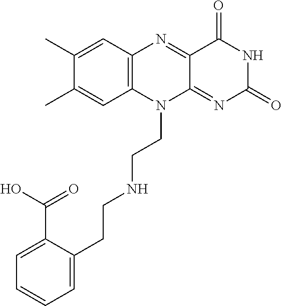 | 434 | 3.5 | Method C | Prepared using the procedure of Example 70 | 2-{2-[(2-{7,8-dimethyl-2,4- dioxo-2H,3H,4H,10H- benzo[g]pteridin-10- yl}ethyl)amino]ethyl} benzoic acid |
| 163 | 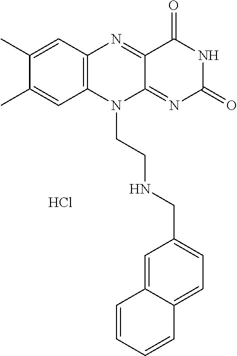 | 426 | 3.87 | Method C | Prepared using the procedure of Example 71 | 7,8-dimethyl-10-{2- [(naphthalen-2- ylmethyl)amino]ethyl}- 2H,3H,4H,10H- benzo[g]pteridine-2,4-dione hydrochloride |
| 164 | 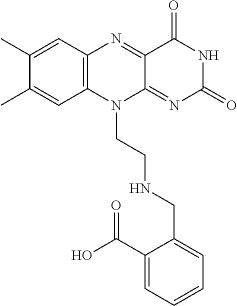 | 420 | 3.36 | Method C | Prepared using the procedure of Example 71 | 2-{[(2-{7,8-dimethyl-2,4- dioxo-2H,3H,4H,10H- benzo[g]pteridin-10- yl}ethyl)amino]methyl} benzoic acid |
| 165 | 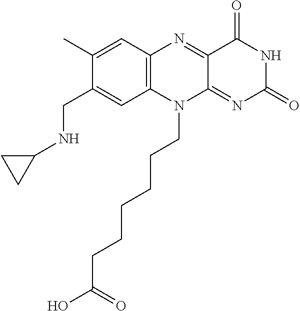 | 426 | 3.17 | Method C | Prepared using the procedure of Example 86 | 7-{8- [(cyclopropylamino)methyl]- 7-methyl-2,4-dioxo- 2H,3H,4H,10H- benzo[g]pteridin-10- yl}heptanoic acid |
| 166 | 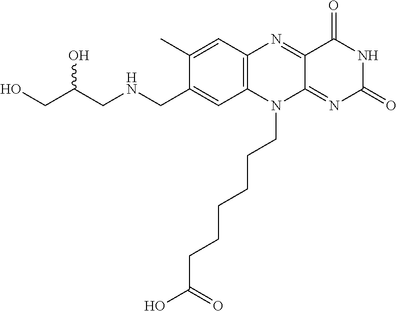 | 460.3 | 1.77 | Method C | Prepared using the procedure of Example 81 | 7-(8-{[(2,3- dihydroxypropyl)amino]meth- yl}-7-methyl-2,4-dioxo- 2H,3H,4H,10H- benzo[g]pteridin-10- yl)heptanoic acid |
| 167 | 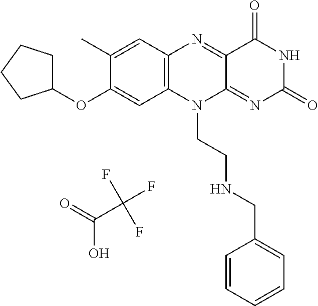 | 446.1 | 4.35 | Method C | Prepared using the procedure of Example 74 | 10-[2-(benzylamino)ethyl]- 8-(cyclopentyloxy)-7- methyl-2H,3H,4H,10H- benzo[g]pteridine-2,4-dione; 2,2,2-trifluoroacetic acid |
| 168 | 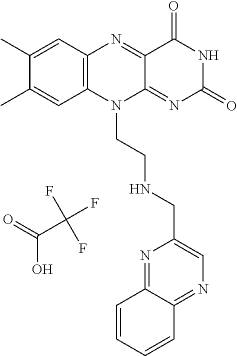 | 428.1 | 3.55 | Method C | Prepared using the procedure of Example 71 | 2,2,2-trifluoroacetic acid; 7,8-dimethyl-10-{2- [(quinoxalin-2- ylmethyl)amino]ethyl}- 2H,3H,4H,10H- benzo[g]pteridine-2,4-dione |
| 169 | 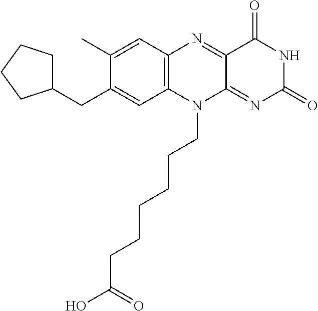 | 439.1 | 4.92 | Method C | Prepared using the procedure of Example 88 | 7-[8-(cyclopentylmethyl)-7- methyl-2,4-dioxo- 2H,3H,4H,10H- benzo[g]pteridin-10- yl]heptanoic acid |
| 170 | 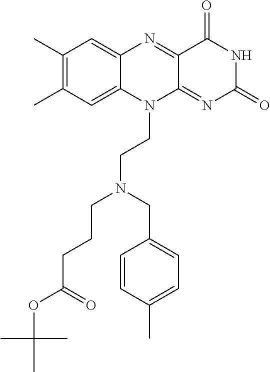 | 532.1 | 4.45 | Method C | Prepared using the procedure of Example 75 | tert-butyl 4-[(2-{7,8- dimethyl-2,4-dioxo- 2H,3H,4H,10H- benzo[g]pteridin-10- yl}ethyl)[(4- methylphenyl)methyl]amino] butanoate |
| 171 | 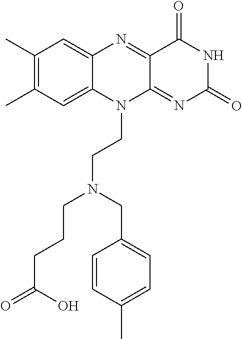 | 476.1 | 3.74 | Method C | Prepared using the procedure of Example 75 | 4-[(2-{7,8-dimethyl-2,4- dioxo-2H,3H,4H,10H- benzo[g]pteridin-10- yl}ethyl)[(4- methylphenyl)methyl]amino] butanoic acid |
| 172 | 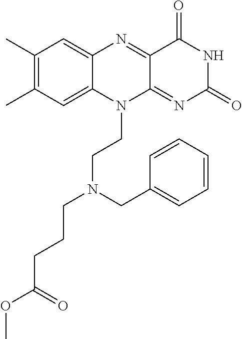 | 476.0 | 3.81 | Method C | Prepared using the procedure of Example 25 | methyl 4-[benzyl(2-{7,8- dimethyl-2,4-dioxo- 2H,3H,4H,10H- benzo[g]pteridin-10- yl}ethyl)amino]butanoate |
| 173 | 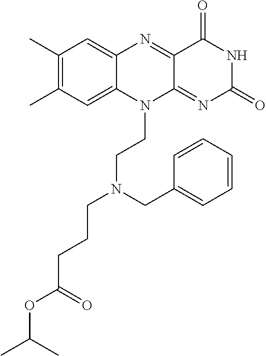 | 504.1 | 4.14 | Method C | Prepared using the procedure of Example 25 | propan-2-yl 4-[benzyl(2- {7,8-dimethyl-2,4-dioxo- 2H,3H,4H,10H- benzo[g]pteridin-10- yl}ethyl)amino]butanoate |
| 174 | 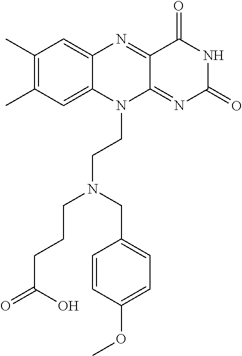 | 491.9 | 3.61 | Method C | Prepared using the procedure of Example 75 | 4-[(2-{7,8-dimethyl-2,4- dioxo-2H,3H,4H,10H- benzo[g]pteridin-10- yl}ethyl)[(4- methoxyphenyl)methyl] amino]butanoic acid |
| 175 | 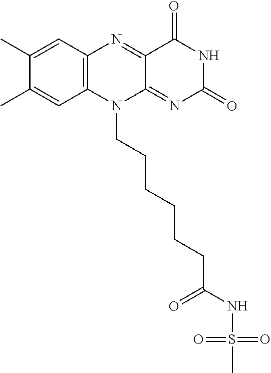 | 447.9 | 4.02 | Method C | Prepared using the procedure of Example 69 | 7-{7,8-dimethyl-2,4-dioxo- 2H,3H,4H,10H- benzo[g]pteridin-10-yl}-N- methanesulfonylheptanamide |
| 176 | 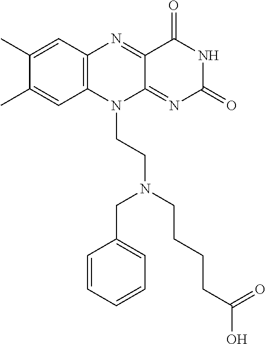 | 476 | 3.66 | Method C | Prepared using the procedure of Example 75 | 5-[benzyl(2-{7,8-dimethyl- 2,4-dioxo-2H,3H,4H,10H- benzo[g]pteridin-10- yl}ethyl)amino]pentanoic acid |
| 177 | 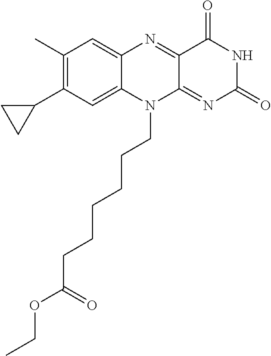 | 425.2 | 3.7 | System D | Prepared using the procedure of Example 65, Step 3 | ethyl 7-{8-cyclopropyl-7- methyl-2,4-dioxo- 2H,3H,4H,10H- benzo[g]pteridin-10- yl}heptanoate |
| 178 | 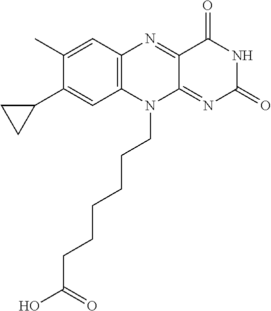 | 397.1 | 3.1 | System D | Prepared using the procedure of Example 65 | 7-{8-cyclopropyl-7-methyl- 2,4-dioxo-2H,3H,4H,10H- benzo[g]pteridin-10- yl}heptanoic acid |
| 179 | 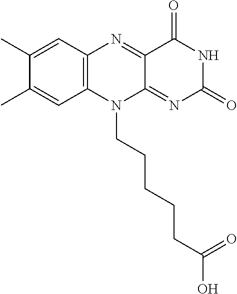 | 357 | 2.75 | Method C | Prepared using the procedure of Example 44 | 6-{7,8-dimethyl-2,4-dioxo- 2H,3H,4H,10H- benzo[g]pteridin-10- yl}hexanoic acid |
| 180 | 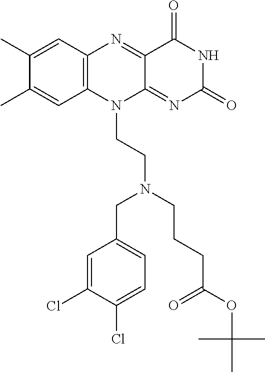 | 586 | 4.82 | Method C | Prepared using the procedure of Example 75 | tert-butyl 4-{[(3,4- dichlorophenyl)methyl](2- {7,8-dimethyl-2,4-dioxo- 2H,3H,4H,10H- benzo[g]pteridin-10- yl}ethyl)amino}butanoate |
| 181 | 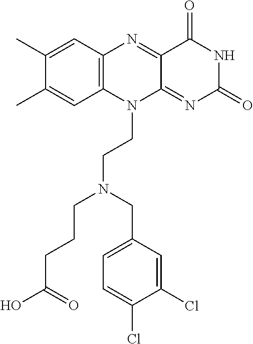 | 529.9 | 8.85 | Method B | Prepared using the procedure of Example 75 | 4-{[(3,4- dichlorophenyl)methyl](2- {7,8-dimethyl-2,4-dioxo- 2H,3H,4H,10H- benzo[g]pteridin-10- yl}ethyl)amino}butanoic acid |
| 182 | 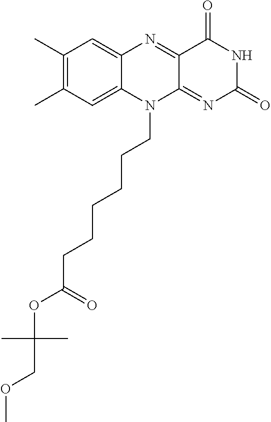 | 456.9 [M + H]+ 479.1 [M + Na]+ | 4.84 | Method C | Prepared using a procedure similar to that of Example 24, Step 2 (stirred at 70° C.) | 1-methoxy-2-methylpropan- 2-yl 7-{7,8 dimethyl-2,4- dioxo-2H,3H,4H,10H- benzo[g]pteridin-10- yl}heptanoate |
| 183 | 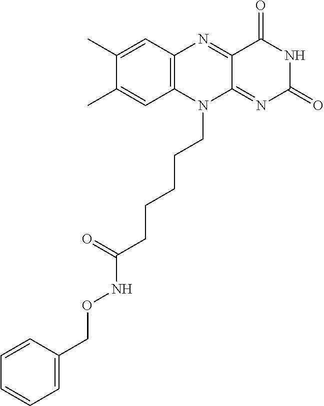 | 462.1 | 4.33 | Method C | Prepared using the procedure of Example 66 | N-(benzyloxy)-6-{7,8- dimethyl-2,4-dioxo- 2H,3H,4H,10H- benzo[g]pteridin-10- yl}hexanamide |
| 184 | 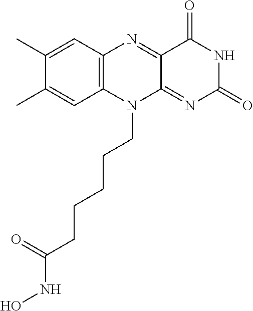 | 372 | 3.56 | Method C | Prepared using the procedure of Example 67 | 6-{7,8-dimethyl-2,4-dioxo- 2H,3H,4H,10H- benzo[g]pteridin-10-yl}-N- hydroxyhexanamide |
| 185 | 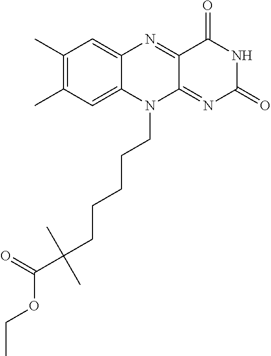 | 427.1 | 5.05 | Method C | Prepared using the procedure of Example 77 | ethyl 7-{7,8-dimethyl-2,4- dioxo-2H,3H,4H,10H- benzo[g]pteridin-10-yl}-2,2- dimethylheptanoate |
| 186 | 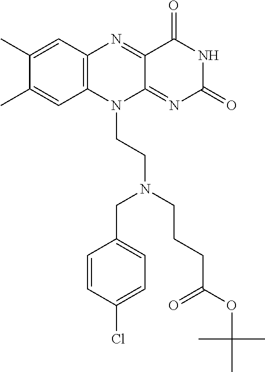 | 552 | 4.59 | Method C | Prepared using the procedure of Example 75 | tert-butyl 4-{[(4- chlorophenyl)methyl](2- {7,8-dimethyl-2,4-dioxo- 2H,3H,4H,10H- benzo[g]pteridin-10- yl}ethyl)amino}butanoate |
| 187 | 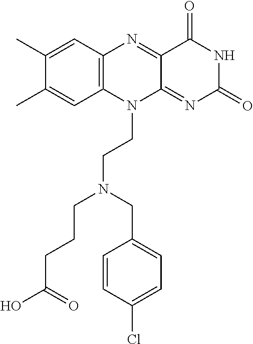 | 495.9 | 3.88 | Method C | Prepared using the procedure of Example 75 | 4-{[(4- chlorophenyl)methyl](2- {7,8-dimethyl-2,4-dioxo- 2H,3H,4H,10H- benzo[g]pteridin-10- yl}ethyl)amino}butanoic acid |
| 188 | 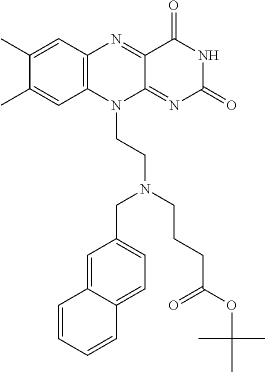 | 568.1 | 4.57 | Method C | Prepared using the procedure of Example 75 | tert-butyl 4-[(2-{7,8- dimethyl-2,4-dioxo- 2H,3H,4H,10H- benzo[g]pteridin-10- yl}ethyl)(naphthalen-2- ylmethyl)amino]butanoate |
| 189 | 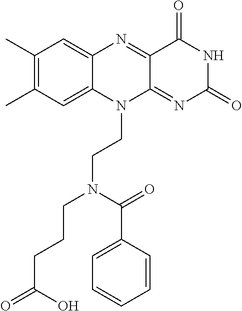 | 475.9 | 3.81 | Method C | Prepared using the procedure of Example 52, Step 1-2 using the product of Example 20 as starting material | 4-[N-(2-{7,8-dimethyl-2,4- dioxo-2H,3H,4H,10H- benzo[g]pteridin-10- yl}ethyl)-1- phenylformamido]butanoic acid |
| 190 | 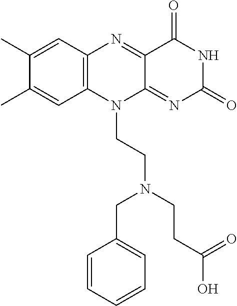 | 448 | 3.58 | Method C | Prepared using the procedure of Example 75 | 3-[benzyl(2-{7,8-dimethyl- 2,4-dioxo-2H,3H,4H,10H- benzo[g]pteridin-10- yl}ethyl)amino]propanoic acid |
| 191 | 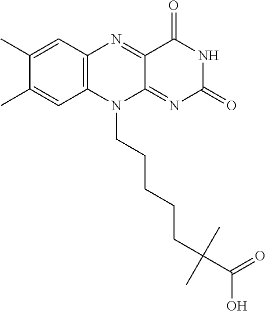 | 399.1 | 3.07 | Method C | Prepared using the procedure of Example 78 | 7-{7,8-dimethyl-2,4-dioxo- 2H,3H,4H,10H- benzo[g]pteridin-10-yl}-2,2- dimethylheptanoic acid |
| 192 | 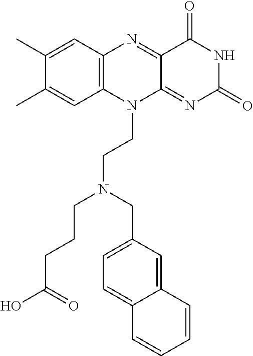 | 512.1 | 4.28 | Method C | Prepared using the procedure of Example 75 | 4-[(2-{7,8-dimethyl-2,4- dioxo-2H,3H,4H,10H- benzo[g]pteridin-10- yl}ethyl)(naphthalen-2- ylmethyl)amino]butanoic acid |
| 193 | 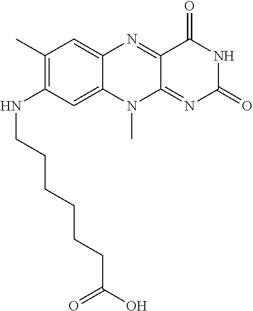 | 386.3 | 2.55 | Method C | Prepared using the procedure of Example 76 | 7-({7,10-dimethyl-2,4- dioxo-2H,3H,4H,10H- benzo[g]pteridin-8- yl}amino)heptanoic acid |
| 194 | 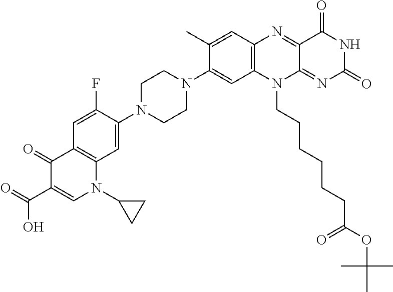 | 742.1 | 5.65 | Method C | Prepared using the procedure of Example 85 | 7-(4-{10-[7-(tert-butoxy)-7- oxoheptyl]-7-methyl-2,4- dioxo-2H,3H,4H,10H- benzo[g]pteridin-8- yl}piperazin-1-yl)-1- cyclopropyl-6-fluoro-4-oxo- 1,4-dihydroquinoline-3- carboxylic acid |
| 195 | 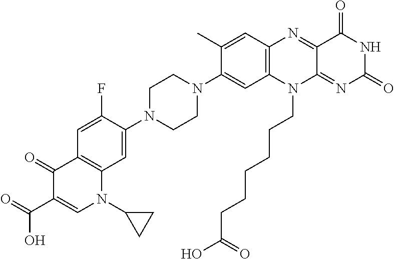 | 686.3 | 5.72 | Method C | Prepared using the procedure of Example 85 | 7-{4-[10-(6-carboxyhexyl)- 7-methyl-2,4-dioxo- 2H,3H,4H,10H- benzo[g]pteridin-8- yl}piperazin-1-yl}-1- cyclopropyl-6-fluoro-4-oxo- 1,4-dihydroquinoline-3- carboxylic acid |
| 196 | 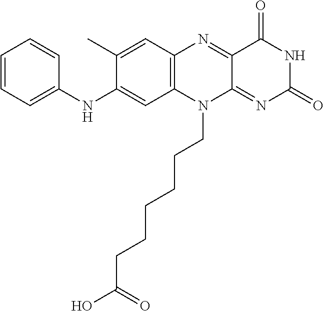 | 448.2 | 4.54 | Method C | Prepared using the procedure of Example 4, Step 7 | 7-[7-methyl-2,4-dioxo-8- (phenylamino)- 2H,3H,4H,10H- benzo[g]pteridin-10- yl]heptanoic acid |
| 197 | 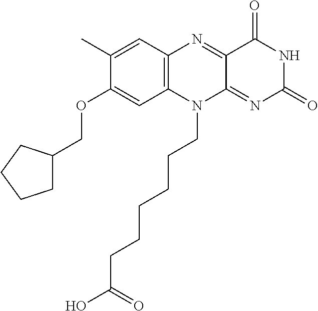 | 455.2 | 5.67 | Method C | Prepared using the procedure of Example 84 | 7-[8-(cyclopentylmethoxy)- 7-methyl-2,4-dioxo- 2H,3H,4H,10H- benzo[g]pteridin-10- yl]heptanoic acid |
| 198 | 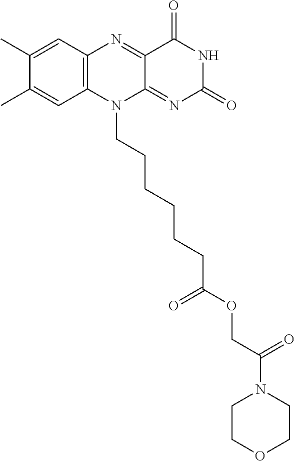 | 498.1 | 4.58 | Method C | Prepared using the procedure similar to that of Example 69 (strirred at 65° C.) | 2-(morpholin-4-yl)-2- oxoethyl 7-{7,8-dimethyl- 2,4-dioxo-2H,3H,4H,10H- benzo[g]pteridin-10- yl}heptanoate |
| 199 | 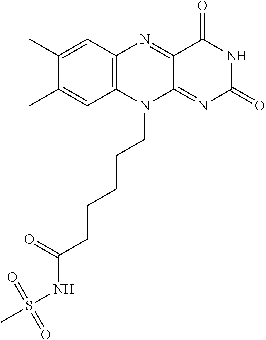 | 434.1 | 2.48 | Method C | Prepared using the procedure of Example 68 | 6-{7,8-dimethyl-2,4-dioxo- 2H,3H,4H,10H- benzo[g]pteridin-10-yl}-N- methanesulfonylhexanamide |
| 200 | 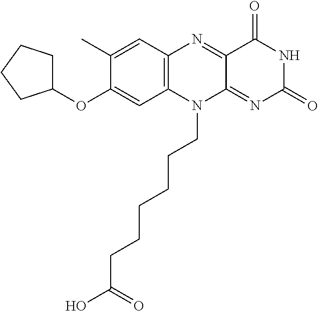 | 441.1 | 5.31 | Method C | Prepared using the procedure of Example 83 | 7-[8-(cyclopentyloxy)-7- methyl-2,4-dioxo- 2H,3H,4H,10H- benzo[g]pteridin-10- yl]heptanoic acid |
| 201 | 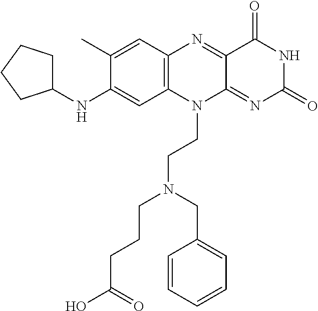 | 531.1 | 4.27 | Method C | Prepared using the procedure of Example 89 | 4-[benzyl({2-[8- (cyclopentylamino)-7- methyl-2,4-dioxo- 2H,3H,4H,10H- benzo[g]pteridin-10- yl]ethyl})amino]butanoic acid |
| 202 | 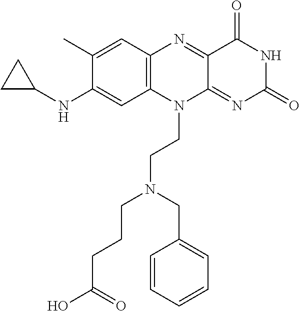 | 503.1 | 3.82 | Method C | Prepared using the procedure of Example 89 | 4-[benzyl({2-[8- (cyclopropylamino)-7- methyl-2,4-dioxo- 2H,3H,4H,10H- benzo[g]pteridin-10- yl]ethyl})amino]butanoic acid |
| 203 | 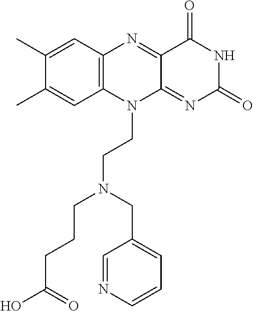 | 463 | 3.54 | Method C | Prepared using the procedure of Example 75 | 4-[(2-{7,8-dimethyl-2,4- dioxo-2H,3H,4H,10H- benzo[g]pteridin-10- yl}ethyl)(pyridin-3- ylmethyl)amino]butanoic acid |
| 204 | 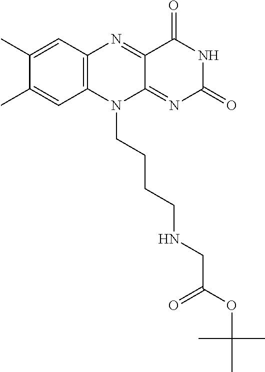 | 428.1 | 4.07 | Method C | Prepared using the procedure of Example 91, Steps 1-2 | tert-butyl 2-[(4-{7,8- dimethyl-2,4-dioxo- 2H,3H,4H,10H- benzo[g]pteridin-10- yl}butyl)amino]acetate |
| 205 | 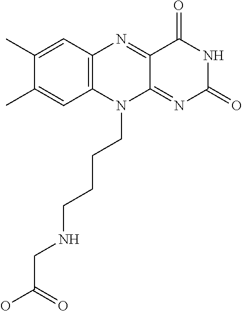 | 372.1 | 3.48 | Method C | Prepared using the procedure of Example 91 | 2-[(4-{7,8-dimethyl-2,4- dioxo-2H,3H,4H,10H- benzo[g]pteridin-10- yl}butyl)amino]acetic acid |
| 206 | 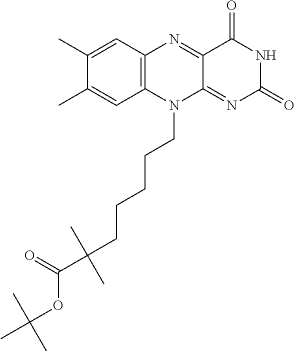 | 455 | 6.24 | Method C | Prepared using a procedure similar to that of Example 24, Step 2 [reaction conducted at 60° C.] | tert-butyl 7-{7,8-dimethyl- 2,4-dioxo-2H,3H,4H,10H- benzo[g]pteridin-10-yl}-2,2- dimethylheptanoate |
| 207 | 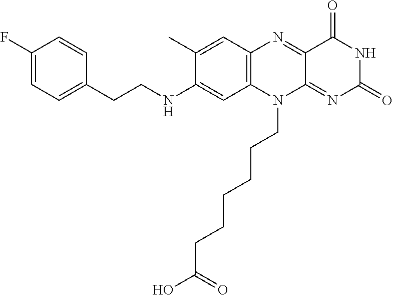 | 494.3 | 3.15 | Method C | Prepared using the procedure of Example 79, Step 1-3 | 7-(8-{[2-(4- fluorophenyl)ethyl]amino}- 7-methyl-2,4-dioxo- 2H,3H,4H,10H- benzo[g]pteridin-10- yl)heptanoic acid |
| 208 | 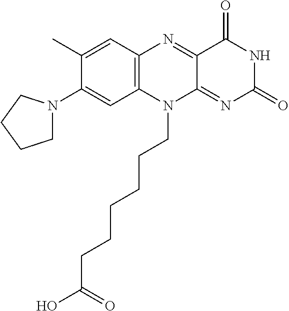 | 426.3 | 2.84 | Method C | Prepared using the procedure of Example 79, Step 1-3 | 7-[7-methyl-2,4-dioxo-8- (pyrrolidin-1-yl)- 2H,3H,4H,10H- benzo[g]pteridin-10- yl]heptanoic acid |
| 209 | 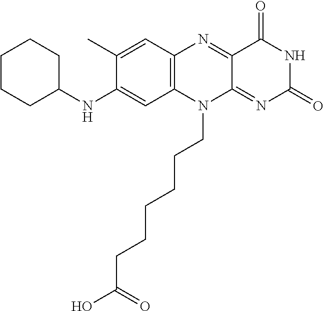 | 454.1 | 4.96 | Method C | Prepared using the procedure of Example 79, Step 1-3 | 7-[8-(cyclohexylamino)-7- methyl-2,4-dioxo- 2H,3H,4H,10H- benzo[g]pteridin-10- yl]heptanoic acid |
| 210 | 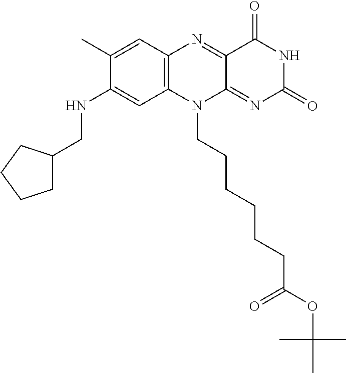 | 510 | 6.67 | Method B | Prepared using the procedure of Example 79, Step 1-3 | tert-butyl 7-{8- [(cyclopentylmethylamino]- 7-methyl-2,4-dioxo- 2H,3H,4H,10H- benzo[g]pteridin-10- yl}heptanoate |
| 211 | 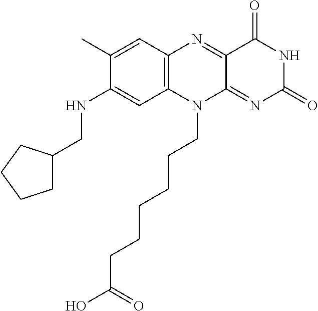 | 454.2 | 5 | Method C | Prepared using the procedure of Example 79, Step 1-3 | 7-{8- [(cyclopentylmethyl)amino]- 7-methyl-2,4-dioxo- 2H,3H,4H,10H- benzo[g]pteridin-10- yl}heptanoic acid |
| 212 | 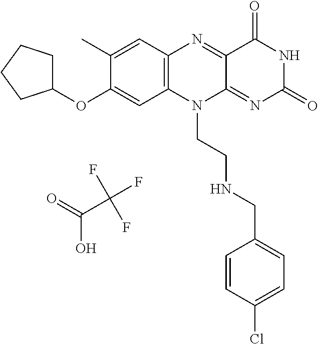 | 480 | 5.03 | Method C | Prepared using the procedure of Example 90 | 10-(2-{[(4- chlorophenyl)methyl]amino} ethyl)-8-(cyclopentyloxy)- 7-methyl-2H,3H,4H,10H- benzo[g]pteridine-2,4-dione; 2,2,2-trifluoroacetic acid |
| 213 | 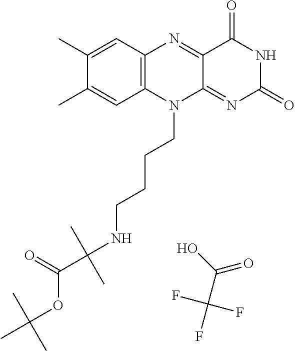 | 456 | 4.38 | Method C | Prepared using the procedure of Example 91 | 2,2,2-trifluoroacetic acid; tert-butyl 2-[(4-{7,8- dimethyl-2,4-dioxo- 2H,3H,4H,10H- benzo[g]pteridin-10- yl}butyl)amino]-2- methylpropanoate |
| 214 | 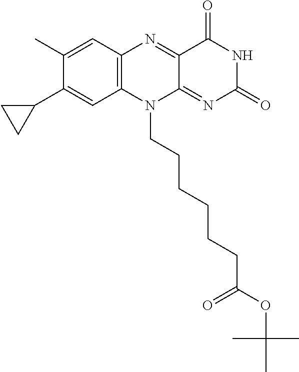 | 453.2 | 4.23 | System C | Prepared using the procedure of Example 92 | tert-butyl 7-{8-cyclopropyl- 7-methyl-2,4-dioxo- 2H,3H,4H,10H- benzo[g]pteridin-10- yl}heptanoate |
| 215 | 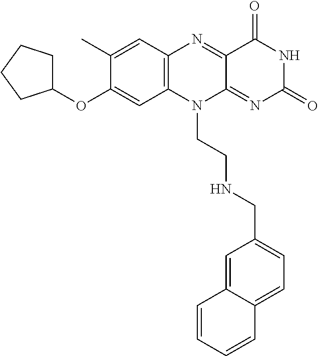 | 496.1 | 7.12 | Method B | Prepared using the procedure of Example 90 | 8-(cyclopentyloxy)-7- methyl-10-{2-[(naphthalen- 2-ylmethyl)amino]ethyl}- 2H,3H,4H,10H- benzo[g]pteridine-2,4-dione |

1. A method for the treatment or prophylaxis of a bacterial infection comprising administering to a patient in need of such treatment an effective amount of a compound selected from:
a) a Compound of formula I(i): 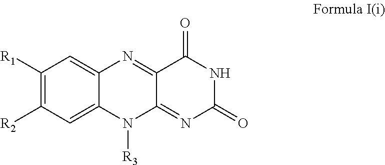
wherein
(i) R₁ is H, C₁₋₈ alkyl or C₃₋₇ cycloalkyl;
(ii) R₂ is H, halo, C₁₋₈alkyl, C₁₋₈alkoxy, —N(R₄)(R₅), C₃₋₇cycloalkyl or C₄₋₇heterocycle wherein said heterocycle is optionally substituted with C₁₋₈alkyl or hydroxyC₁₋₈alkyl; or
(iii) R₁ and R₂ are connected so as to form a cyclic ring structure optionally containing one or more heteroatoms selected from N, O and S;
(iv) R₃ is H or C₁₋₈ alkyl, wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₀), —CN, —C(O)OR₉, —C(O)N(H)(R₈), —OR₁₀, —C(O)N(R₆)(R₇), and —N(R₆)(R₇);
(v) R₄ and R₅ are independently selected from H, C₃₋₇ cycloalkyl, C₄₋₇heterocycle, and C₁₋₈alkyl wherein said alkyl is optionally substituted with one or more groups selected from —OR₁₁, —C(O)OR₉, —N(R₆)(R₇), C₁₋₈alkoxyl, C₆₋₁₀aryl, C₅₋₁₀ heteroaryl wherein said aryl or heteroaryl are optionally substituted with halo, and C₄₋₇heterocycle wherein said heterocycle is optionally substituted with C₁₋₈alkyl;
(vi) R₆ and R₇ are independently selected from H, C₁₋₈alkyl, —C₁₋₈alkyl-OR₁₁, —C(O)OR₉, —C₁₋₈alkyl-C(O)OR₉, —C₁₋₈alkyl(amine)-C(O)OR₉, C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-C₁₋₈alkyl and aryl wherein said aryl and alkyl are optionally substituted with —COOR₉;
(vii) R₈ is H, C₁₋₈alkyl, OR₁₁ or —OBn;
(viii) R₉ and R₁₇ are independently selected from H, C₁₋₈alkyl, —C₁₋₄alkyl-OC(O)R₁₂, phenyl and Bn wherein said phenyl and Bn are optionally substituted with one or more halo or C₁₋₄alkoxy;
(ix) R₁₀ is H, C₁₋₈alkyl, C₁₋₈alkyl-OR₁₁, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl, or aryl wherein said aryl and alkyl are optionally substituted with —COOR₉, or —C₁₋₄alkyl-OC(O)R₁₂;
(x) R₁₁ is H, or —C₁₋₄alkyl-OC(O)R₁₂;
(xi) R₁₂ is C₁₋₈alkyl or OC₁₋₈alkyl;
(xii) R₁₃, R₁₄, R₁₅ and R₁₆ are independently selected from H, C₁₋₈alkyl, and —C₁₋₈alkyl-COOR₁₈, wherein the alkyl group of C₁₋₈-alkyl-COOR₁₈ is optionally substituted with hydroxyC₁₋₈alkyl, carboxyC₁₋₈alkyl;
(xiii) R₁₈ is H or C₁₋₈alkyl;
in free, pharmaceutically acceptable salt or prodrug form;
b) a Compound of formula I: 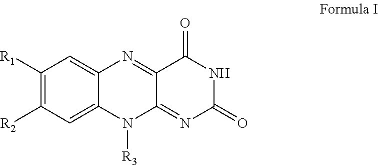
wherein
(i) R₁ is H, C₁₋₈alkyl or C₃₋₇cycloalkyl;
(ii) R₂ is H, halo, C₁₋₈ alkyl, C₁₋₈alkoxy, —N(R₄)(R₅), C₃₋₇cycloalkyl or C₄₋₇heterocycle wherein said heterocycle is optionally substituted with C₁₋₈alkyl or hydroxyC₁₋₈alkyl;
(iii) R₃ is H or C₁₋₈ alkyl, wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇); or
(iv) R₁ and R₂ are connected so as to form a cyclic ring structure optionally containing one or more heteroatoms selected from N, O and S;
(v) R₄ and R₅ are independently selected from H, C₃₋₇cycloalkyl, C₄₋₇heterocycle, and C₁₋₈ alkyl wherein said alkyl is optionally substituted with one or more groups selected from —OR₁₁, —C(O)OR₉, —N(R₆)(R₇), C₁₋₈alkoxyl, C₆₋₁₀ aryl, C₅₋₁₀heteroaryl wherein said aryl or heteroaryl are optionally substituted with halo, and C₄₋₇heterocycle wherein said heterocycle is optionally substituted with C₁₋₈ alkyl;
(vi) R₆ and R₇ are independently selected from H, C₁₋₈ alkyl, —C₁₋₈ alkyl-OR₁₁, —C(O)OR₉, —C₁₋₈alkyl-C(O)OR₉, —C₁₋₈alkyl(amine)-C(O)OR₉, —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl and aryl wherein said aryl is optionally substituted with —COOR₉;
(vii) R₈ is H, C₁₋₈alkyl, OR₁₁ or —OBn;
(viii) R₉ and R₁₇ are independently selected from H, C₁₋₈alkyl, —C₁₋₄alkyl-OC(O)R₁₂, phenyl and Bn wherein said phenyl and Bn are optionally substituted with one or more halo or C₁₋₄alkoxy;
(ix) R₁₀ is H, C₁₋₈ alkyl, —C₁₋₈alkyl-OR₁₁, —C₁₋₈alkyl-C(O)OR₉, —C₁₋₈alkyl(amine)-C(O)OR₉, —C₁₋₈alkyl-C(O)N(H)R₈, —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl, or aryl wherein said aryl is optionally substituted with —COOR₉, or —C₁₋₄alkyl-OC(O)R₁₂;
(x) R₁₁ is H, or —C₁₋₄alkyl-OC(O)R₁₂;
(xi) R₁₂ is C₁₋₈alkyl or —OC₁₋₈alkyl;
(xii) R₁₃, R₁₄, R₁₅ and R₁₆ are independently selected from H, C₁₋₈alkyl, and —C₁₋₈alkyl-COOR₁₈, wherein the alkyl group of C₁₋₈alkyl-COOR₁₈ is optionally substituted with hydroxyC₁₋₈alkyl, carboxyC₁₋₈alkyl;
(xiii) R₁₈ is H or C₁₋₈alkyl;
in free, pharmaceutically acceptable salt or prodrug form;
c) a Compound of formula I(ii): 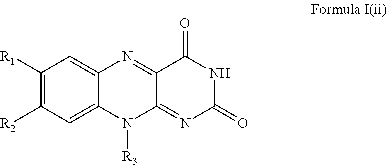
wherein
(i) R₁ is H, C₁₋₈ alkyl;
(ii) R₂ is H, halo, C₁₋₈alkyl, C₁₋₈alkoxy, —N(R₄)(R₅);
(iii) R₃ is C₁₋₈ alkyl, wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇);
(iv) R₄ and R₅ are independently selected from H, C₃₋₇ cycloalkyl, and C₁₋₈alkyl wherein said alkyl is optionally substituted with one or more groups selected from —OR₁₁;
(v) R₆ and R₇ are independently selected from H, C₁₋₈alkyl, —C₁₋₈alkyl-C(O)OR₉, —C₁₋₈alkyl(amine)-C(O)OR₉, —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), 7,8-dimethyl-isoalloxazin-10-yl-C₁₋₈alkyl and aryl wherein said aryl and alkyl are optionally substituted with —COOR₉;
(vi) R₈ is H, C₁₋₈alkyl;
(vii) R₉ and R₁₇ are independently selected from H, C₁₋₈alkyl, ethyl, n —C₁₋₄alkyl-OC(O)R₁₂, phenyl and Bn wherein said phenyl and Bn are optionally substituted with one or more halo or C₁₋₄alkoxy;
(viii) R₁₀ is —C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-C₁₋₈alkyl, or aryl wherein said aryl and alkyl are optionally substituted with —COOR₉, or —C₁₋₄alkyl-OC(O)R₁₂;
(ix) R₁₁ is H, or —C₁₋₄alkyl-OC(O)R₁₂;
(x) R₁₂ is C₁₋₈alkyl;
(xi) R₁₃, R₁₄, R₁₅ and R₁₆ are independently selected from H, C₁₋₈alkyl, and —C₁₋₈alkyl-COOR₁₈, wherein the alkyl group of C₁₋₈alkyl-COOR₁₈ is optionally substituted with hydroxyC₁₋₈alkyl, carboxyC₁₋₈alkyl; and
(xii) R₁₈ is H or C₁₋₈alkyl;
in free, pharmaceutically acceptable salt or prodrug form;
d) a Compound of formula I(iii): 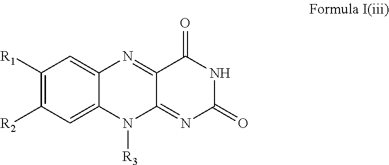
wherein
(i) R₁ is H, C₁₋₈alkyl;
(ii) R₂ is H, halo, C₁₋₈alkyl, C₁₋₈alkoxy, —N(R₄)(R₅);
(iii) R₃ is C₁₋₈ alkyl, wherein the alkyl group is optionally substituted with one or more groups selected from —P(O)(OR₉)(OR₁₇), —P(O)(OR₉)(NR₁₃R₁₄), —P(O)(NR₁₃R₁₄)(NR₁₅R₁₆), —C(O)OR₉, —OR₁₀, —C(O)N(R₆)(R₇), and —N(R₆)(R₇);
(iv) R₄ and R₅ are independently selected from H, C₃₋₇ cycloalkyl, and C₁₋₈alkyl wherein said alkyl is optionally substituted with one or more groups selected from —OH;
(v) R₆ and R₇ are independently selected from H, —C₁₋₈alkyl, C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl, wherein said aryl and alkyl are optionally substituted with —COOR₉;
(vi) R₉ and R₁₇ are independently selected from H, C₁₋₈alkyl, n —C₁₋₄alkyl-OC(O)R₁₂;
(vii) R₁₀ is H, —C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆);
(viii) R₁₂ is C₁₋₈alkyl;
(ix) R₁₃, R₁₄, R₁₅ and R₁₆ are independently selected from H, C₁₋₈alkyl, and —C₁₋₈alkyl-COOR₁₈;
(x) R₁₈ is H or C₁₋₈alkyl;
in free, pharmaceutically acceptable salt or prodrug form;
e) a Compound of formula I(iv): 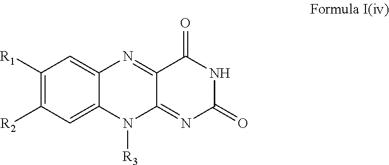
wherein
(i) R₁ is H, C₁₋₈ alkyl;
(ii) R₂ is H, halo, C₁₋₈alkyl, C₁₋₈alkoxy, —N(R₄)(R₅);
(iii) R₃ is C₁₋₈alkyl-N(R₆)(R₇), C₁₋₈alkyl-C(O)N(R₆)(R₇), C₁₋₈alkyl-P(O)(OR₉)(OR₁₇), C₁₋₈alkyl-P(O)(OR₉)(NR₁₃R₁₄), C₁₋₈alkyl-P(O)(NR₁₃R₁₄)(NR₁₅R₁₆), C₁₋₈alkyl-C(O)OR₉, C₁₋₈alkyl-OR₁₀;
(iv) R₄ and R₅ are independently selected from H, C₃₋₇ cycloalkyl, and C₁₋₈alkyl wherein said alkyl is optionally substituted with one or more groups selected from —OR₁₁;
(V) R₆ and R₇ are independently selected from H, C₁₋₈alkyl, —C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl and aryl wherein said aryl and alkyl are optionally substituted with —COOR₉;
(vi) R₉ and R₁₇ are independently selected from H, C₁₋₈alkyl, —C₁₋₄alkyl-OC(O)R₁₂;
(vii) R₁₀ is H, —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl wherein said aryl and alkyl are optionally substituted with —COOR₉;
(viii) R₁₂ is C₁₋₈alkyl;
(ix) R₁₃, R₁₄, R₁₅ and R₁₆ are independently selected from H, C₁₋₈alkyl, and —C₁₋₈alkyl-COOR₁₈;
(x) R₁ g is H or C₁₋₈alkyl;
in free, pharmaceutically acceptable salt or prodrug form;
f) a Compound of formula I(v): 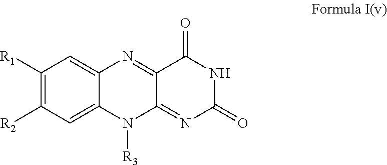
wherein
(i) R₁ is H, C₁₋₈ alkyl;
(ii) R₂ is H, halo, C₁₋₈alkyl, C₁₋₈alkoxy, —N(R₄)(R₅);
(iii) R₃ is C₂alkyl-N(R₆)(R₇), C₁₋₂alkyl-C(O)N(R₆)(R₇), C₁₋₈alkyl-P(O)(OR₉)(OR₁₇), C₁₋₈alkyl-P(O)(OR₉)(NR₁₃R₁₄), C₁₋₈alkyl-P(O)(NR₁₃R₁₄)(NR₁₅R₁₆), C₁₋₈alkyl-C(O)OR₉, C₁₋₈alkyl-OR₁₀;
(iv) R₄ and R₅ are independently selected from H, C₃₋₇ cycloalkyl, and C₁₋₈alkyl wherein said alkyl is optionally substituted with one or more groups selected from —OR₁₁;
(v) R₆ and R₇ are independently selected from —C₁₋₈alkyl-C(O)OR₉, —C₁₋₈alkyl(amine)-C(O)OR₉, C₁₋₈alkyl-P(O)(OR₉)(OR₁₇), —C₁₋₈alkyl-P(O)(OR₉)(NR₁₃R₁₄), —C₁₋₈alkyl-P(O)(NR₃R₁₄)(NR₁₅R₁₆), 7,8-dimethyl-isoalloxazin-10-yl-C₁₋₈alkyl and aryl wherein said aryl and alkyl are optionally substituted with —COOR₉;
(vi) R₉ and R₁₇ are independently selected from H, C₁₋₈alkyl, —C₁₋₄alkyl-OC(O)R₁₂;
(vii) R₁₀ is H, —C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl wherein said aryl and alkyl are optionally substituted with —COOR₉;
(viii) R₁₂ is C₁₋₈alkyl;
(ix) R₁₃, R₁₄, R₁₅ and R₁₆ are independently selected from H, C₁₋₈alkyl, and —C₁₋₈alkyl-COOR₁₈;
(x) R₁₈ is H or C₁₋₈alkyl;
in free, pharmaceutically acceptable salt or prodrug form;
g) a Compound of formula III: 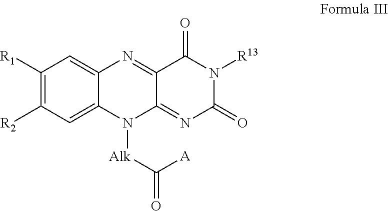
wherein:
(i) Alk is C₁₋₈ alkyl;
(ii) A is —OR₉ or —N(R₁₄)(R₁₅);
(iii) R₉ is H, —C₁₋₈alkyl, haloC₁₋₈alkyl, —C₁₋₄alkyl-OC(O)R₁₂, —C₁₋₄alkyl-O—C₁₋₄alkyl —C₁₋₄ alkyl-C(O)-(morphylin-4-yl), —C₃₋₇cycloalkyl, C₃₋₇cycloalkyl-C₁₋₄alkyl wherein the cycloalkyl is optionally substituted with hydroxy group; aryl or aryl-C₁₋₄alkyl, wherein said aryl is optionally substituted with one or more halo or C₁₋₄alkoxy;
(iv) R₁ is H, C₁₋₈ alkyl;
(v) R₂ is H, halo, —O—C₃₋₇cycloalkyl, —O—C₀₋₇ alkylC₃₋₇cycloalkyl, —N(R₄)(R₅), —(CH₂)—N(R₄)(R₅), —C₀₋₄alkyl-C₃₋₇cycloalkyl, heteroC₃₋₇cycloalkyl, 1-cyclopropyl-6-fluoro-7-[4-piperazin-1-yl]-4-oxo-quinoline-3-carboxylic acid), C₁₋₈alkyl or O—C₁₋₈alkyl, wherein the alkyl group is optionally substituted with one or more halo or hydroxy groups;
(vi) R₄ and R₅ are independently
a. H,
b. —O₀₋₄alkyl-C₃₋₇cycloalkyl,
c. heteroC₃₋₇cycloalkyl,
d. aryl,
e. aryl-C₁₋₈alkyl wherein the aryl is optionally substituted with halo,
f. —(CH₂)₃—N(H)—(CH₂)₄—N(H)—(CH₂)₃—N(H)₂,
g. —C₁₋₈alkyl wherein said alkyl is optionally substituted with one or more hydroxy groups;
(vii) R₁₂ is C₁₋₈alkyl or OC₁₋₈alkyl; and
(viii) R₁₃ is H or C₁₋₄alkyl;
(ix) R₁₄ and R₁₅ are independently H, —OH, —S(O)₂CH₃, —OBn or —C₁₋₄alkyl,
in free, pharmaceutically acceptable salt or prodrug form.
h) a Compound of formula IV: 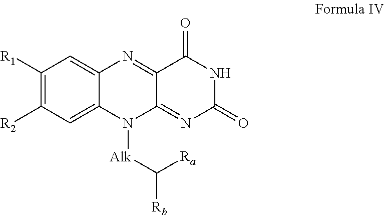
wherein:
(i) Alk is C₁₋₈ alkyl;
(ii) Rₐ and R_{b} are independently H, —C₁₋₄alkyl, —(CH₂)₃C(NH₂)(COOH)CHF₂, —(CH₂)₃N(H)C(═NH)NH₂, —(CH₂)₅NH₂, —(CH₂)₂C(H)(OH)COOH, —C(O)(CH₂)₂COOH, —C₁₋₄alkyl-C(O)OR₉ (e.g., —CH₂CH₂CH₂CH₂C(O)OR₉, —CH₂CH₂CH₂C(O)OR₉, —CH₂CH₂C(O)OR₉ or —CH₂C(O)OR₉, —C(CH₃)(CH₃)C(O)OR₉), —C(O)CH₃, aryl (e.g., phenyl), C(O)-aryl, aryl-C₁₋₄alkyl, heteroaryl, heteroaryl-C₁₋₄alkyl, wherein said aryl and heteroaryl groups are optionally substituted with one or more groups selected from —C(O)OR₉, —NH₂, —S(O)₂NH₂, —CH₂NH₂, halo, C₁₋₄alkoxy, C₁₋₄alkyl;
(iii) R₁ is H, C₁₋₈ alkyl;
(iv) R₂ is H, halo, —O—C₃₋₇cycloalkyl, —N(R₄)(R₅), C₃₋₇cycloalkyl, C₁₋₈alkyl or —O—C₁₋₈alkyl wherein the alkyl group is optionally substituted with one or more halo or hydroxyl groups;
(v) R₄ and R₅ are independently H, C₃₋₇cycloalkyl, C₁₋₈alkyl wherein said alkyl is optionally substituted with one or more hydroxy groups;
(vi) R₉ is H or C₁₋₄alkyl;
(vii) R₁₂ is C₁₋₈alkyl or —OC₁₋₈alkyl,
in free, pharmaceutically acceptable salt or prodrug form;
i) a Compound of formula V: 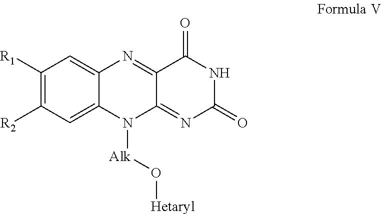
wherein Alk is C₁₋₆alkyl and hetaryl is heteroaryl and R₁ and R₂ are independently H, C₁₋₄alkyl, in free, pharmaceutically acceptable salt or prodrug form;
and
j) a Compound of formula VI: 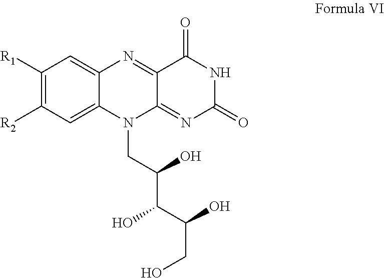
wherein R₁ is H or C₁₋₄alkyl and R₂ is cyano, in free, pharmaceutically acceptable salt or prodrug form,
with the proviso that: (a) when R₁ is methyl and R₂ is chloro, then R₃ is not methyl; (b) when R₁ is H and R₂ is dimethylamine, then R₃ is not H; (c) when R₃ is (2R,3S,4S)-2,3,4,5-tetrahydroxypentyl or 5-dihydrogen phosphate (2R,3S,4S)-trihydroxypentyl, and R₁ is methyl, then R₂ is not methyl; (d) when R₃ is (2R,3S,4S)-2,3,4,5-tetrahydroxypentyl and R₁ is methyl, then R₂ is not dimethylamino; (e) when R₁ is methyl and R₂ is alkoxy, then R₃ is not 2,3,4,5-tetrahydroxypentyl; and (f) when the bacterial infection is an infection by chlamydophila psittacci, then R₃ is not —(CH₂)₂₋₆-phosphate, when R₁ and R₂ are independently selected from a group consisting of C₁₋₅alkyl, C₁₋₅alkoxy, amino, hydrogen and halogen group.

2. (canceled)

3. (canceled)

4. (canceled)

5. (canceled)

6. (canceled)

7. (canceled)

8. (canceled)

9. (canceled)

10. (canceled)

11. (canceled)

12. The method according to claim 1, wherein the infection is a Gram-positive or Gram-negative bacterial infection.

13. The method according to claim 1, wherein the bacterial infection is selected from a group consisting of Moraxella catarrhalis, Klebsiella pneumoniae, Staphylococcus epidermidis, Streptococcus viridans, Enterococcus faecium, Staphylococcus aureus, Bacillus anthracis, Francisella tularensis, Streptococcus pneumoniae, Pseudomonas aeruginosa, Acinetobacter baumannii, Brucella melitensis, Escherichia coli, Haemophilus influenzae, Listeria monocytogenes, Salmonella enterica, Vibrio cholerae, Enterococcus faecalis, Yersinia pestis, Bacillus subtilis, Streptococcus pyogenes and Borrelia burgdorferi.

14. The method according to claim 1, wherein the bacterial infection is Staphylococcus aureus infection.

15. The method according to claim 1, wherein the compound is selected from a group consisting of 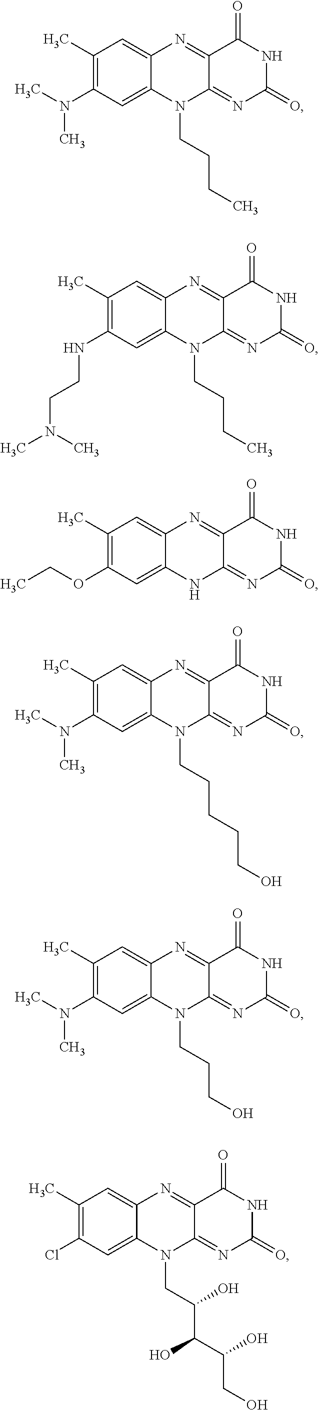 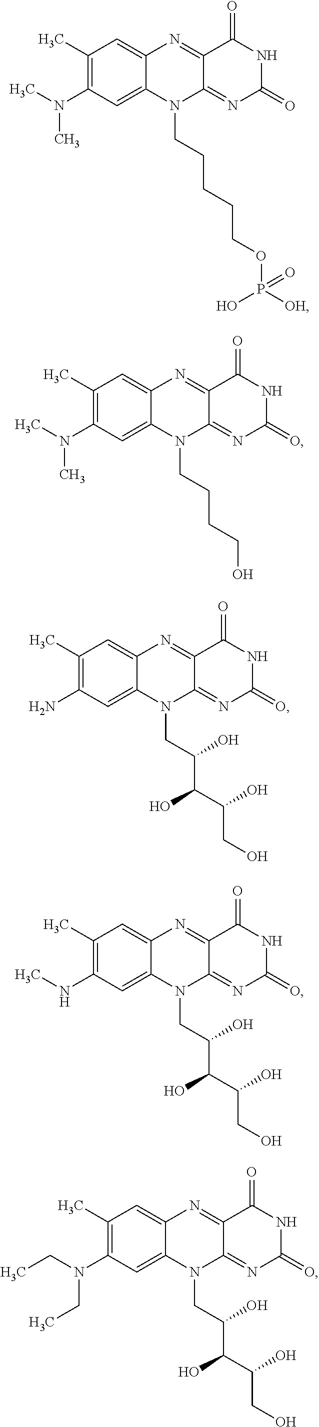 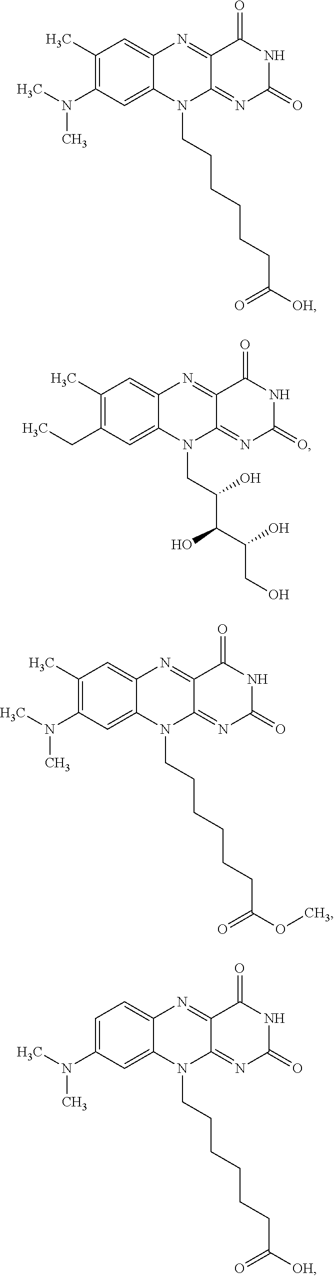 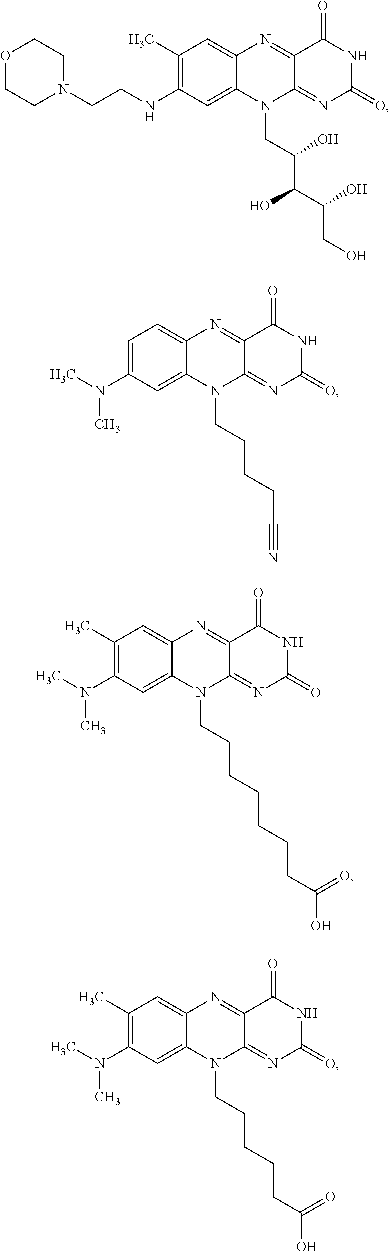 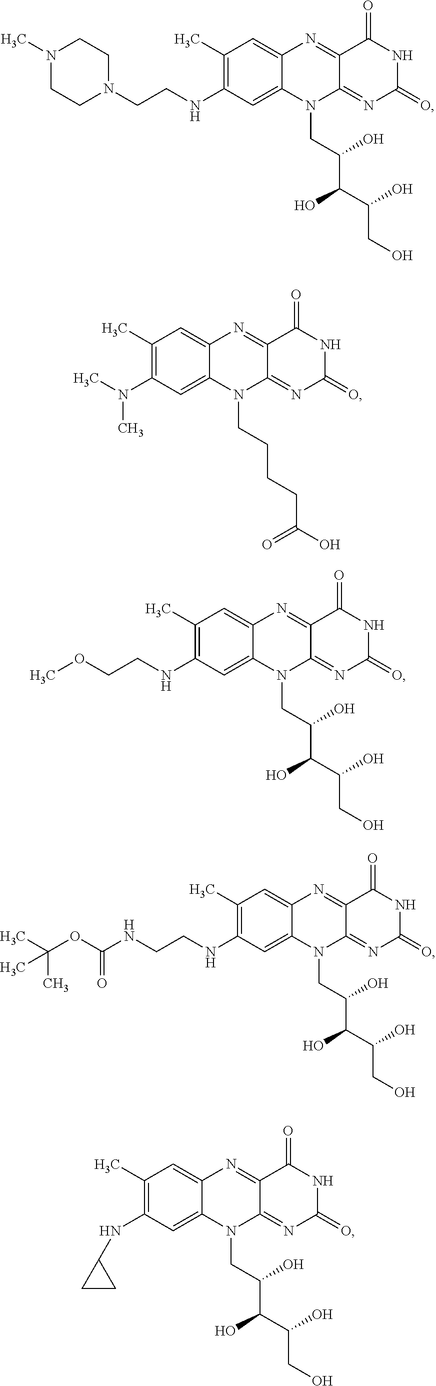 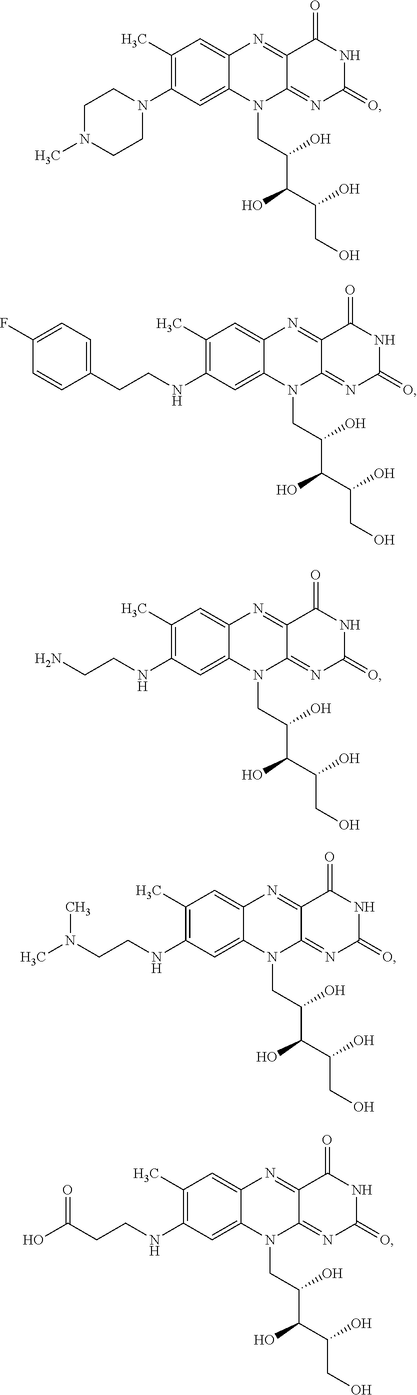 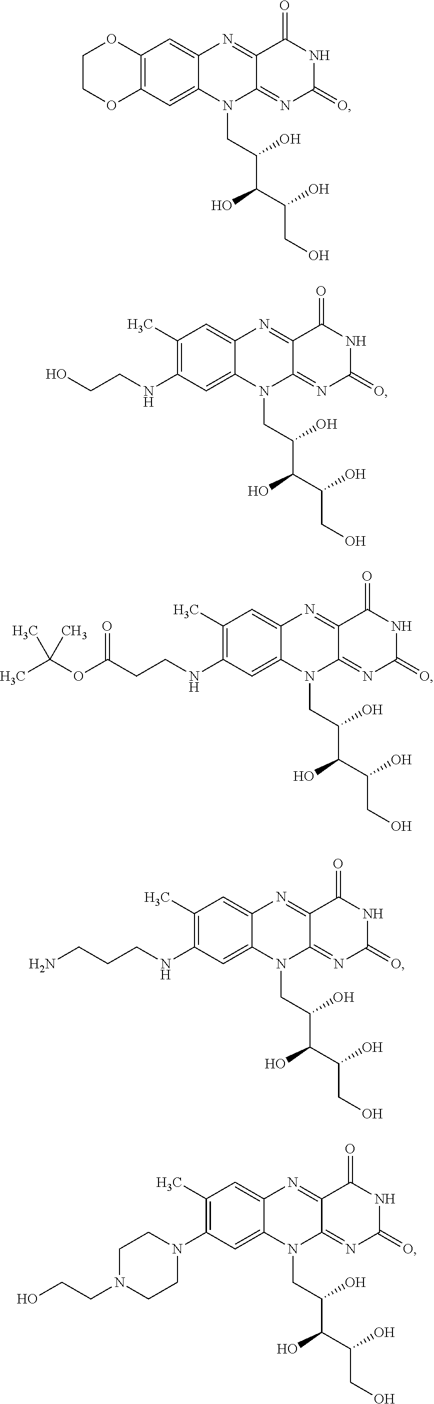 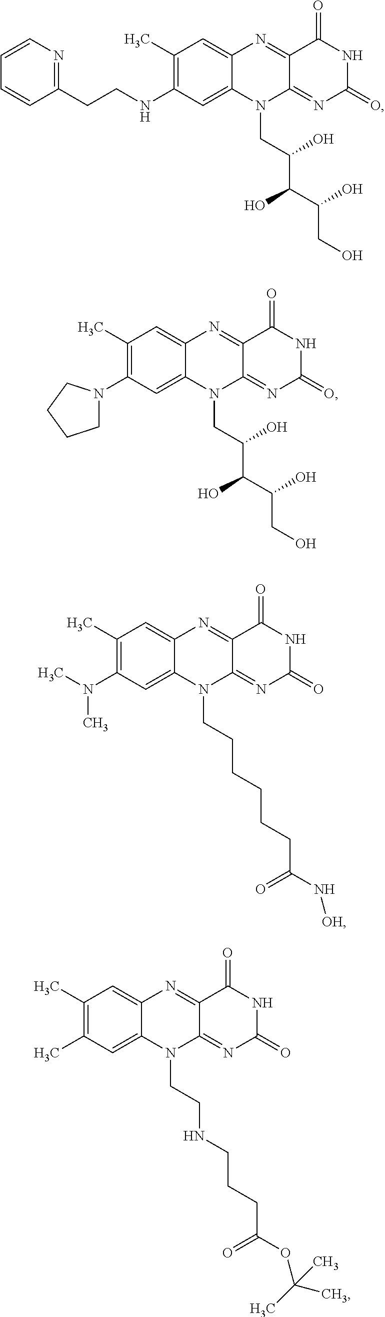 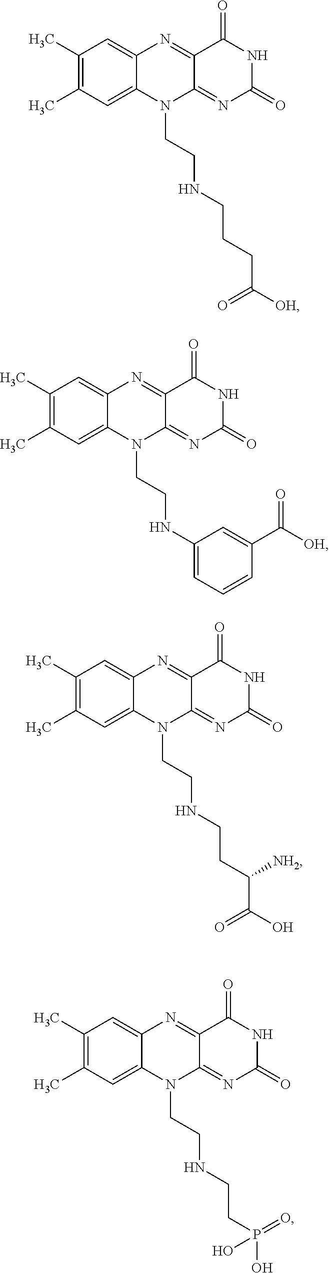 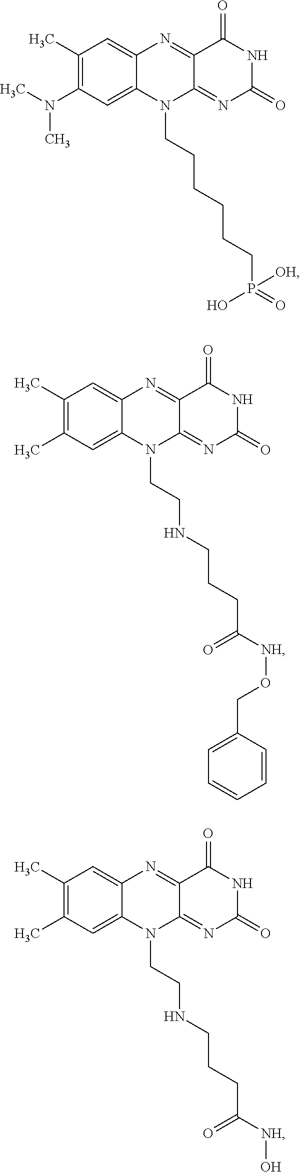 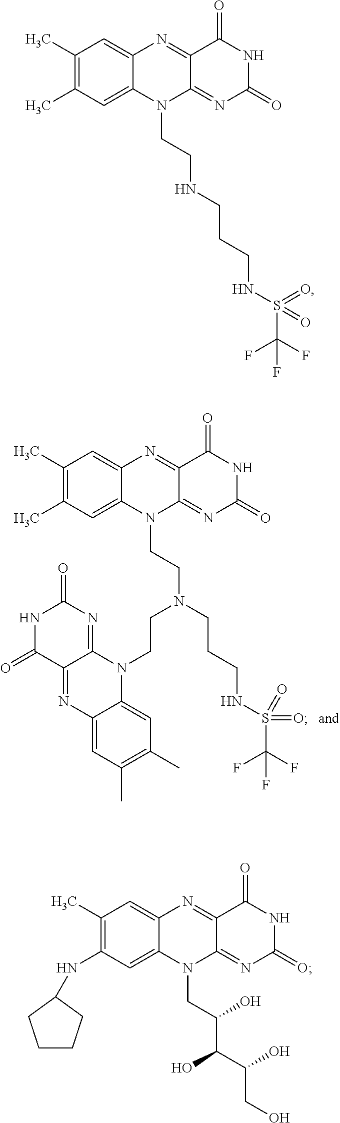
in free, pharmaceutically acceptable salt or prodrug form.

16. The method according to claim 1, wherein the compound is selected from a group consisting of 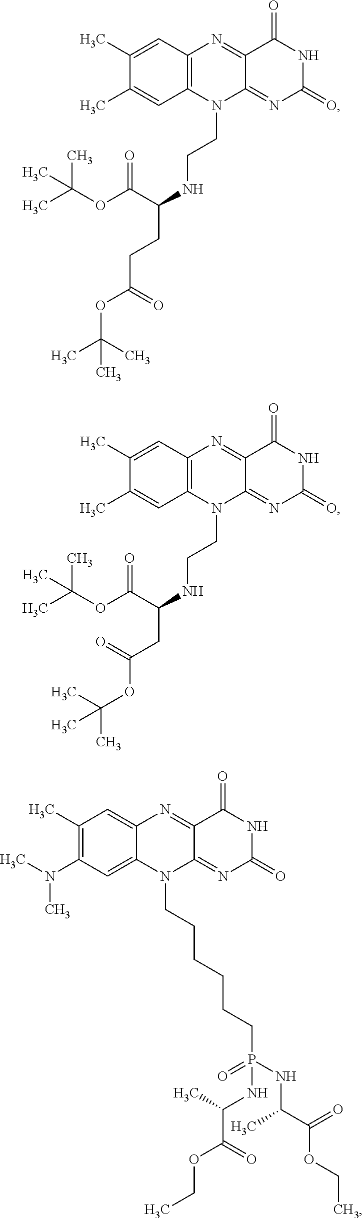 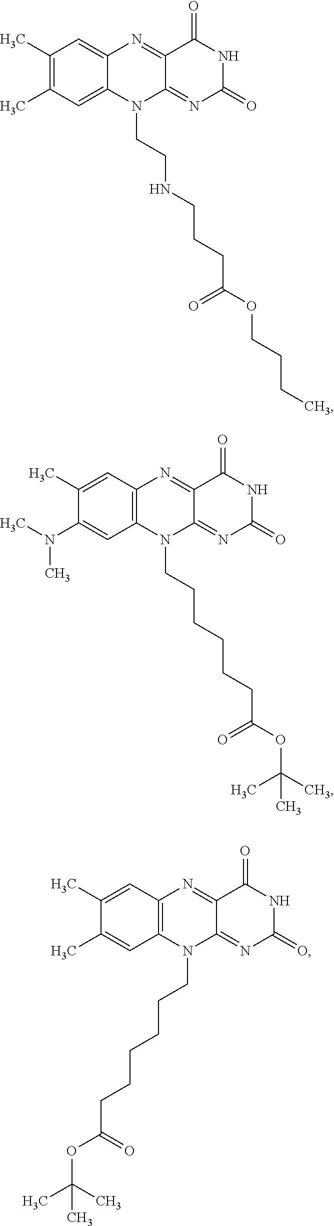 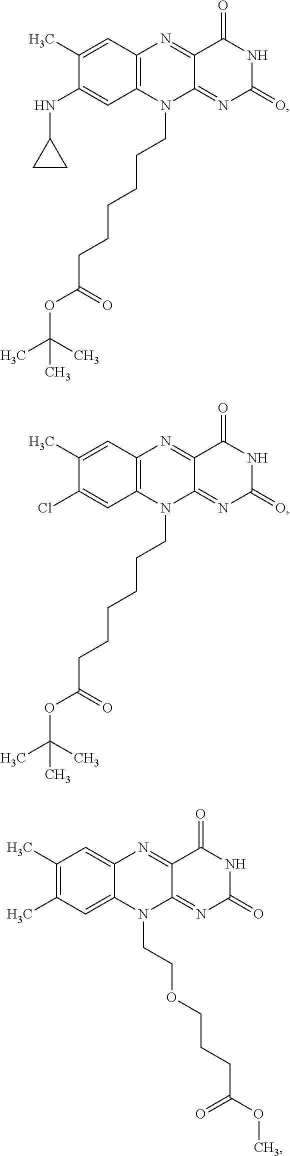 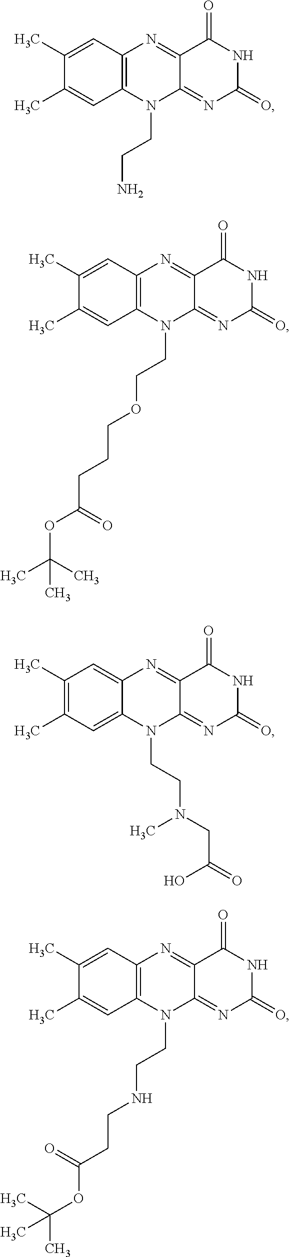 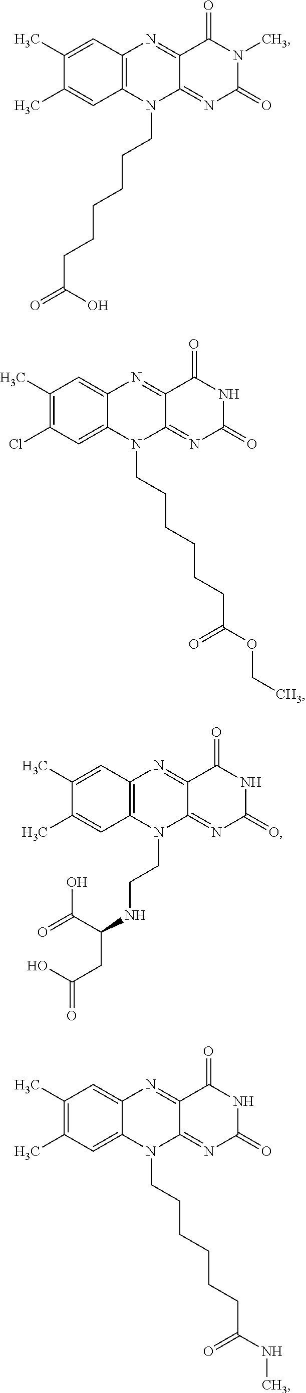 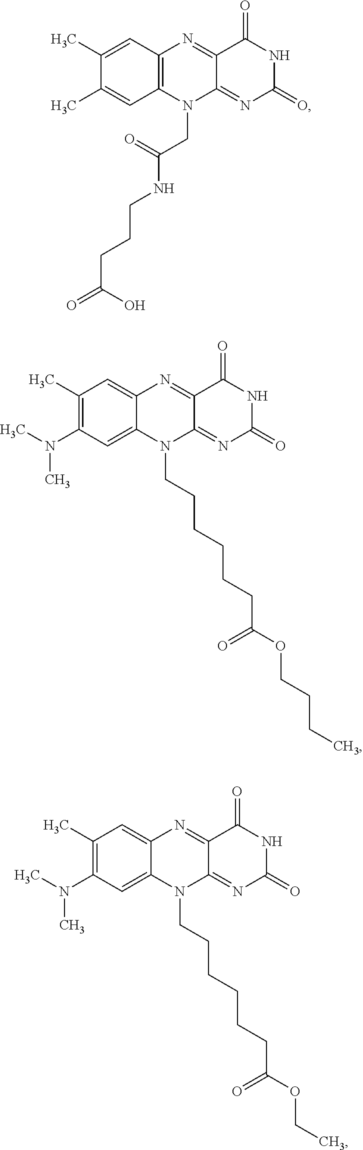 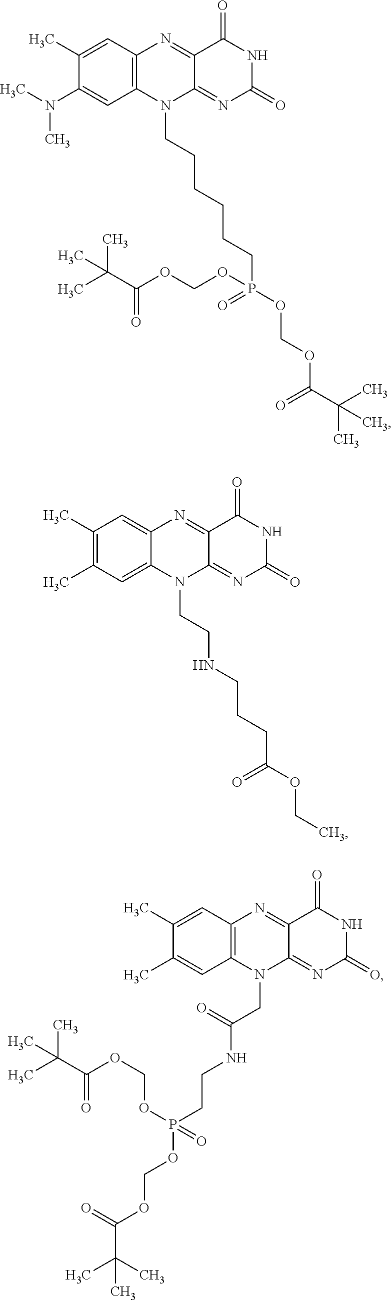 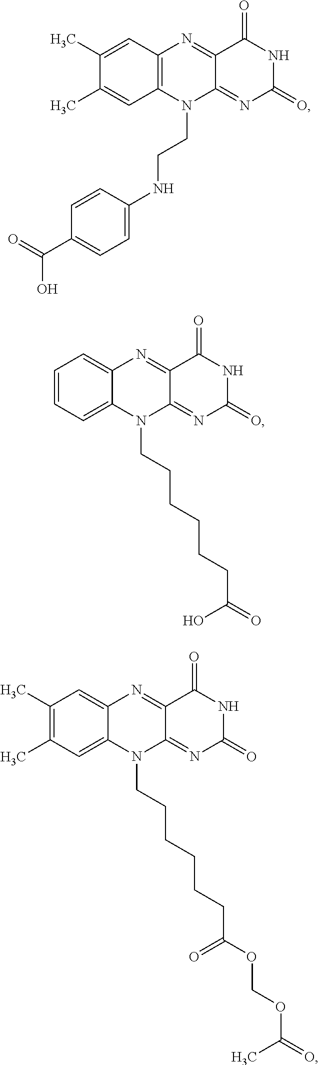 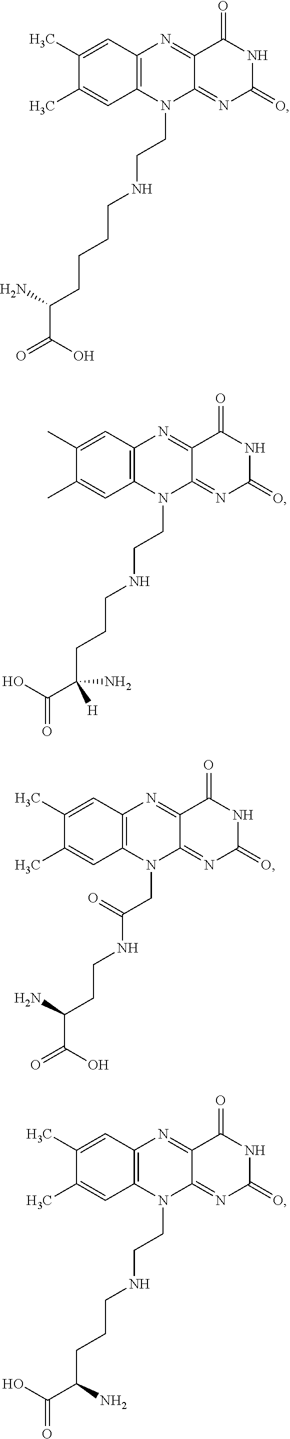 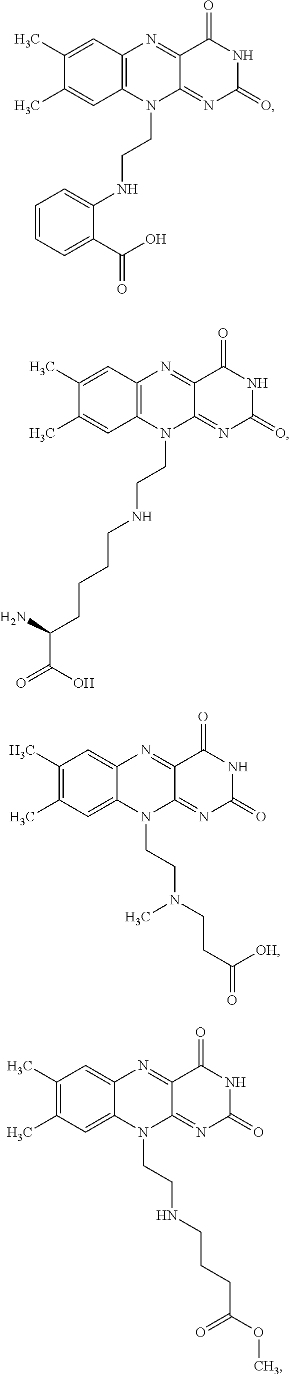 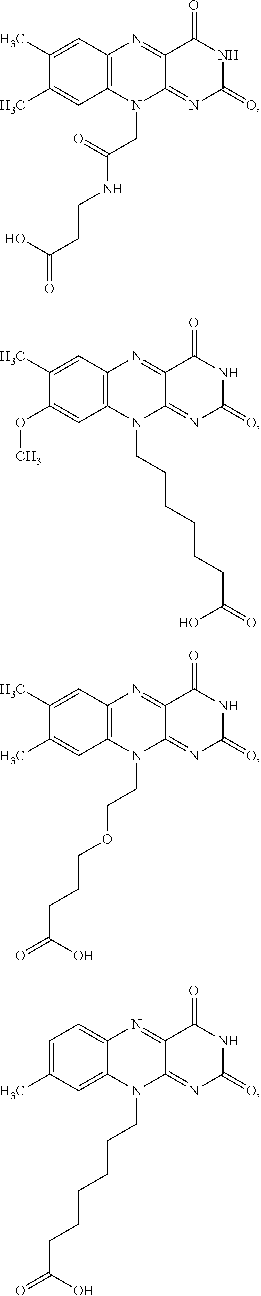 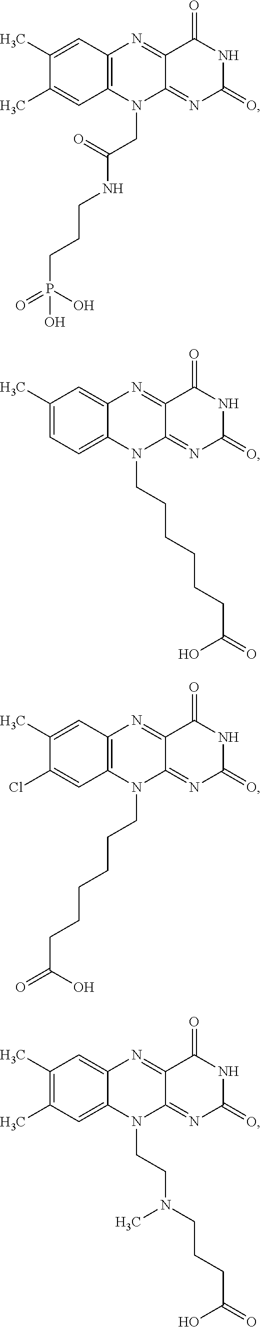 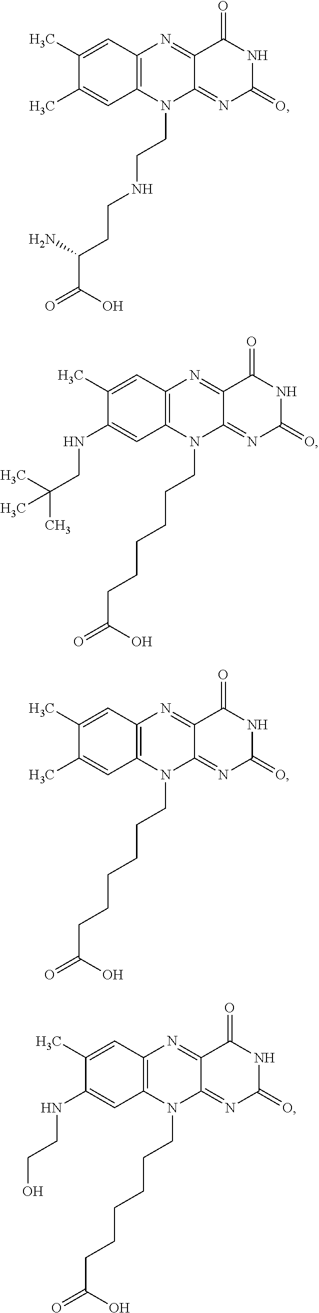 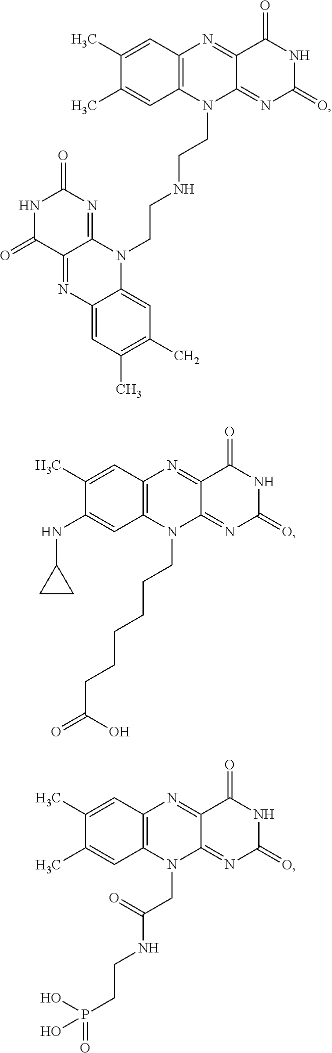
in free, pharmaceutically acceptable salt or prodrug form.

17. The method according to claim 1, wherein the compound is selected from a group consisting of 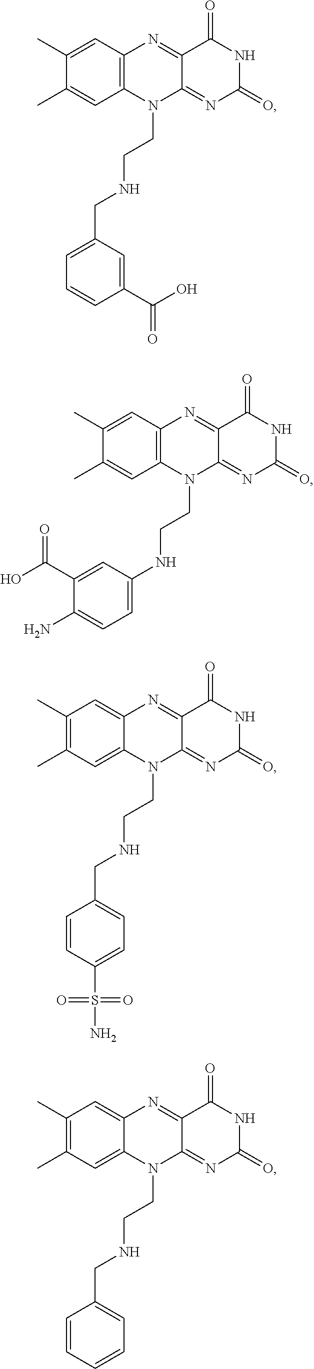 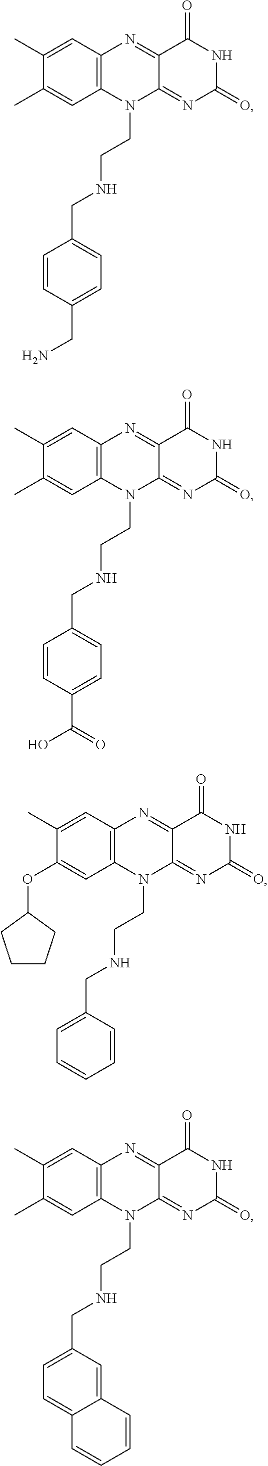 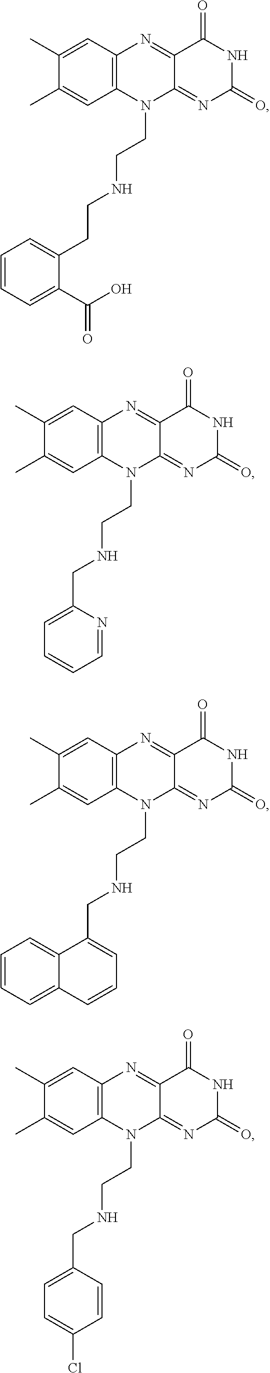 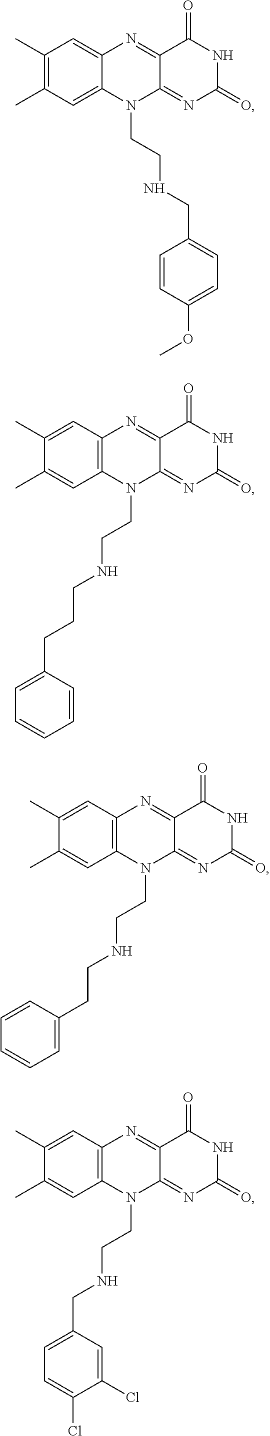 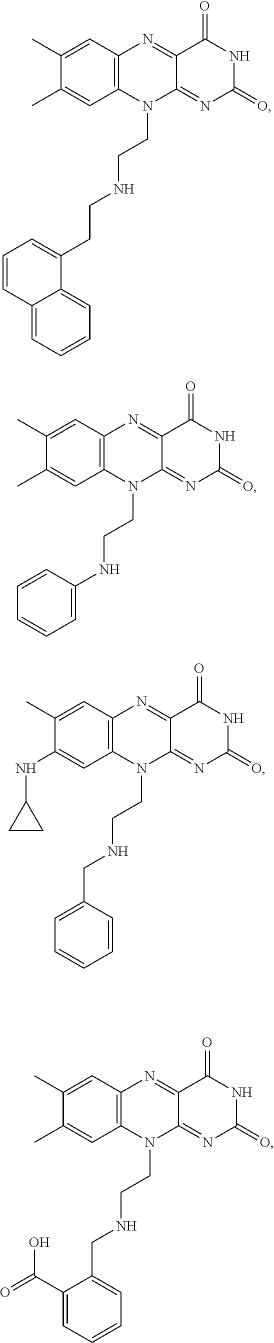 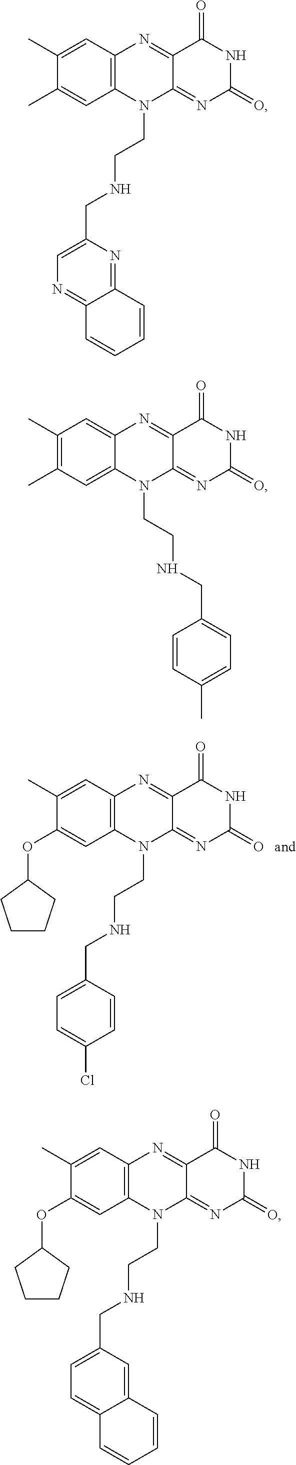
in free, pharmaceutically acceptable salt or prodrug form.

18. The method according to claim 1, wherein the compound is selected from a group consisting of: 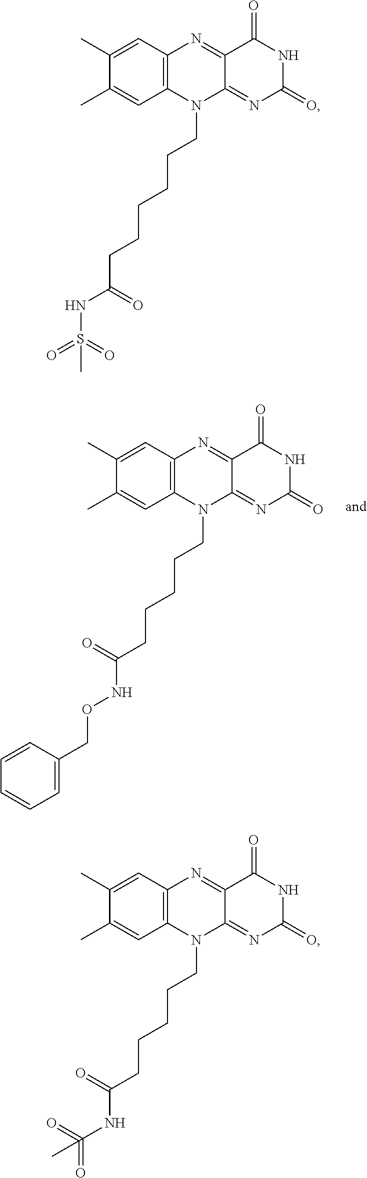
in free, pharmaceutically acceptable salt or prodrug form.

19. The method according to claim 1, wherein the compound is selected from a group consisting of: 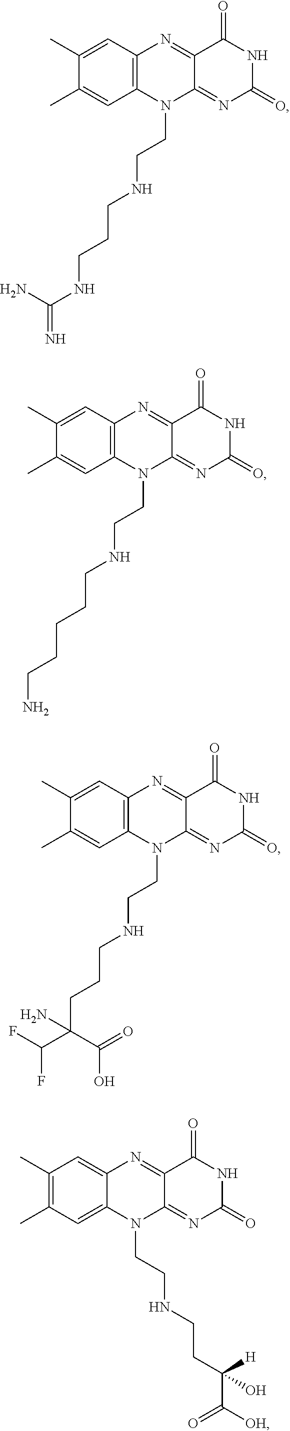 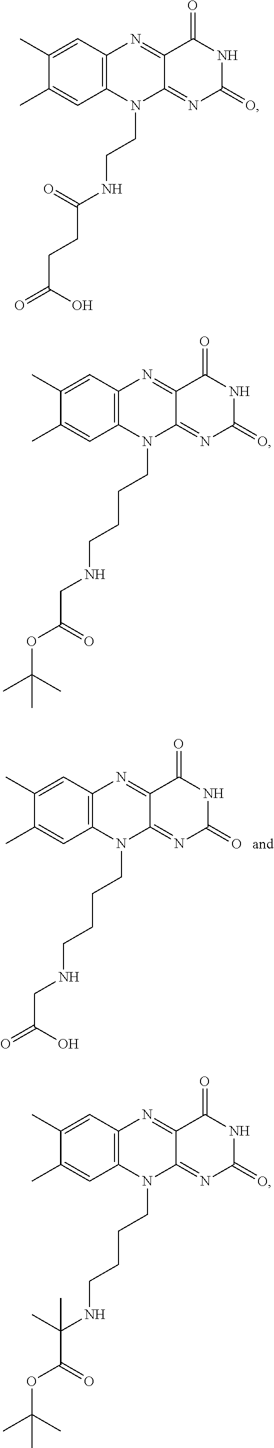
in free, pharmaceutically acceptable salt or prodrug form.

20. The method according to claim 1, wherein the compound is selected from a group consisting of: 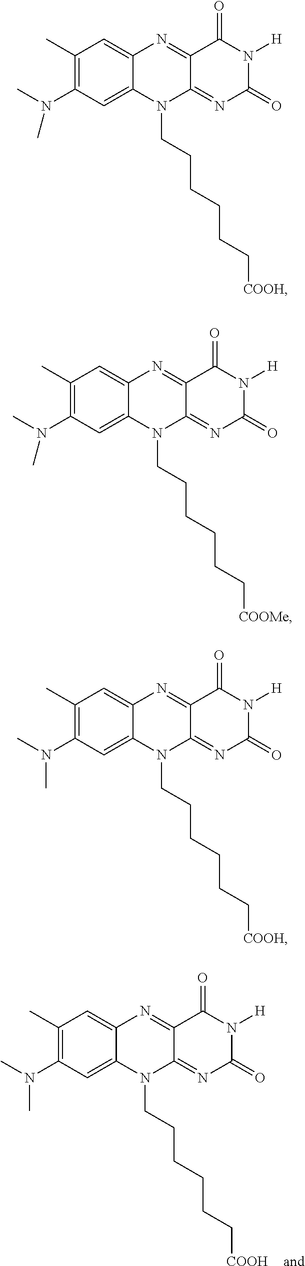 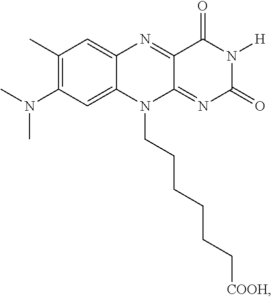
in free, pharmaceutically acceptable salt or prodrug form.

21. The method according to claim 1, wherein the compound is selected from a group consisting of: 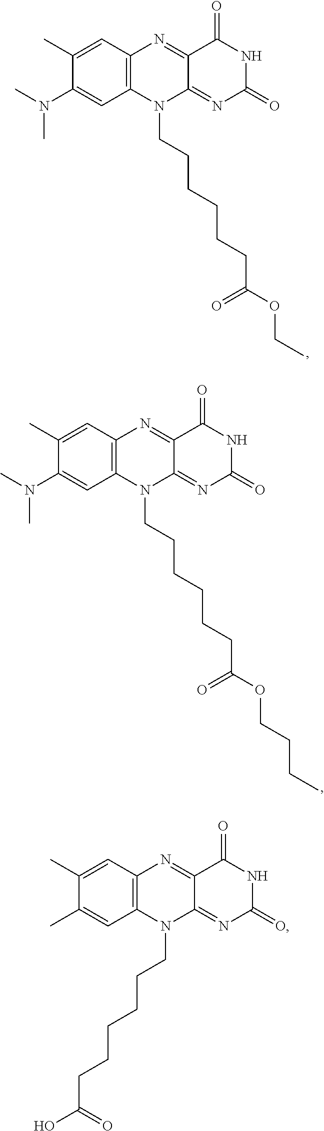 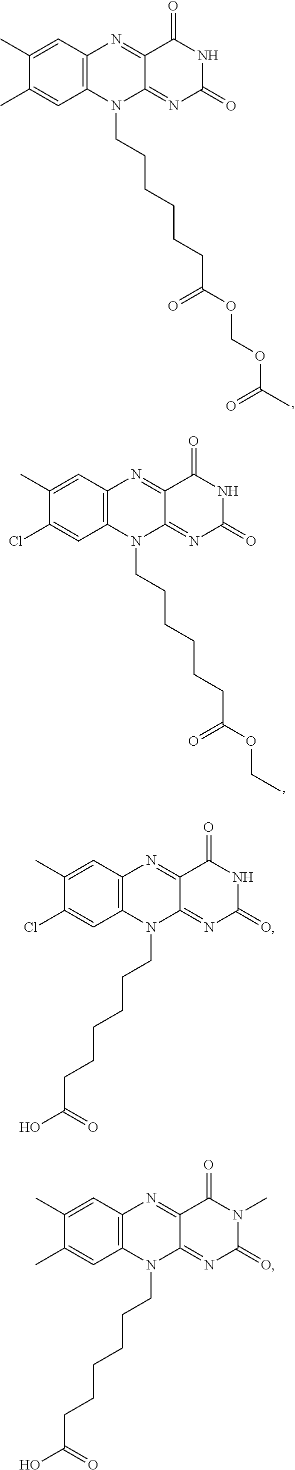 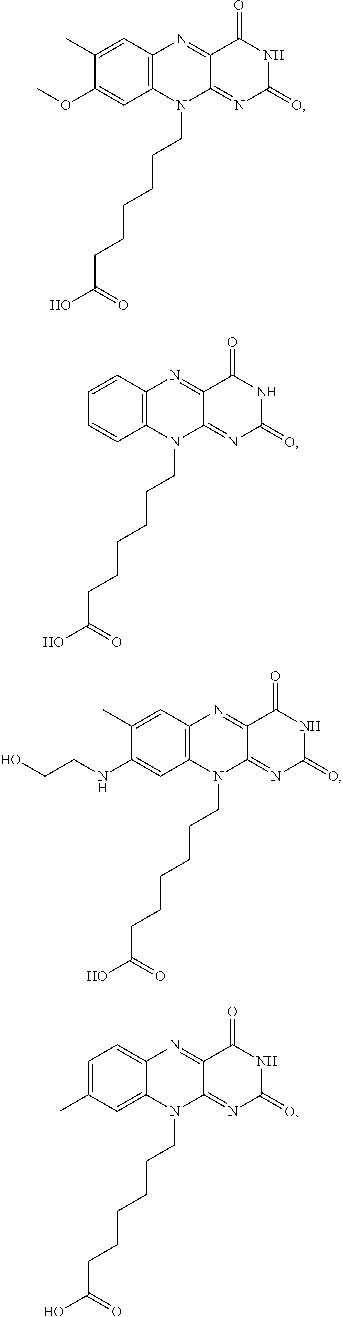 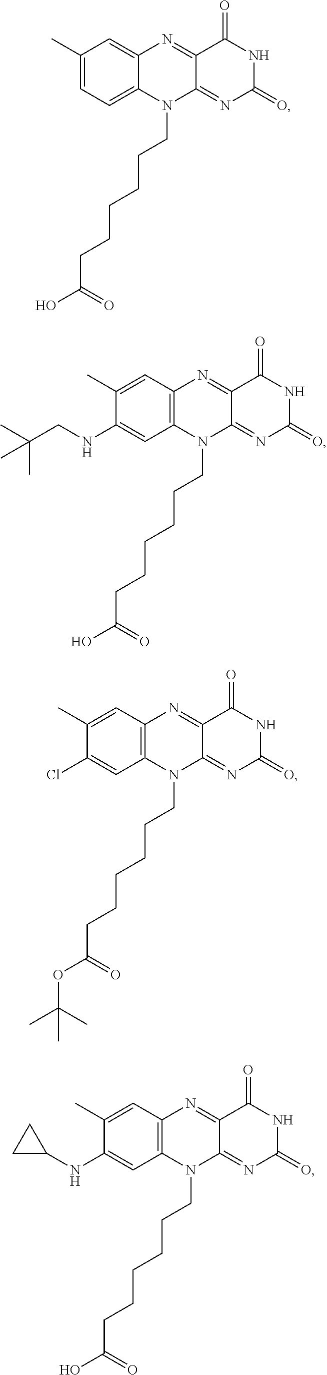 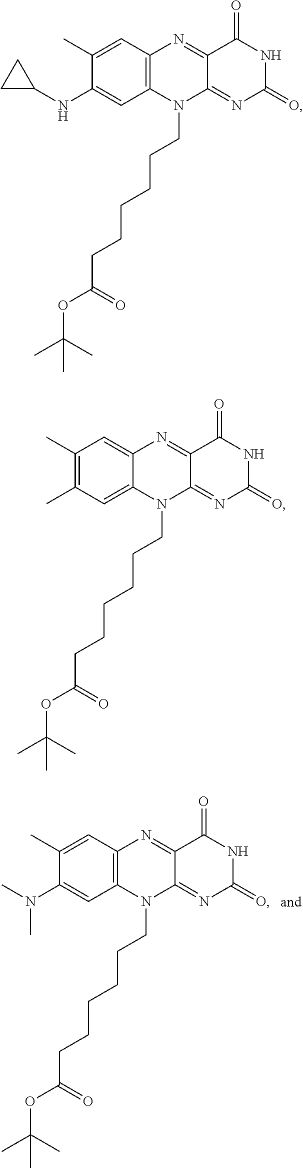 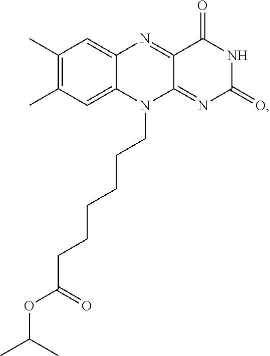
in free, pharmaceutically acceptable salt or prodrug form.

22. The method according to claim 1, wherein the compound is selected from a group consisting of: 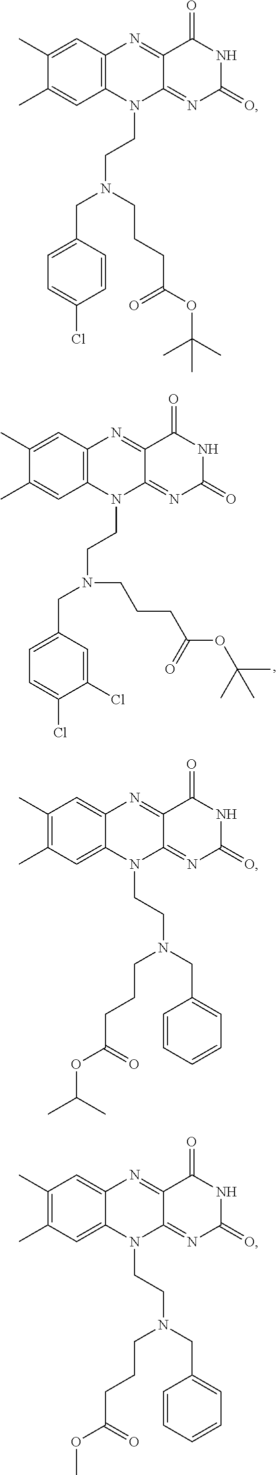 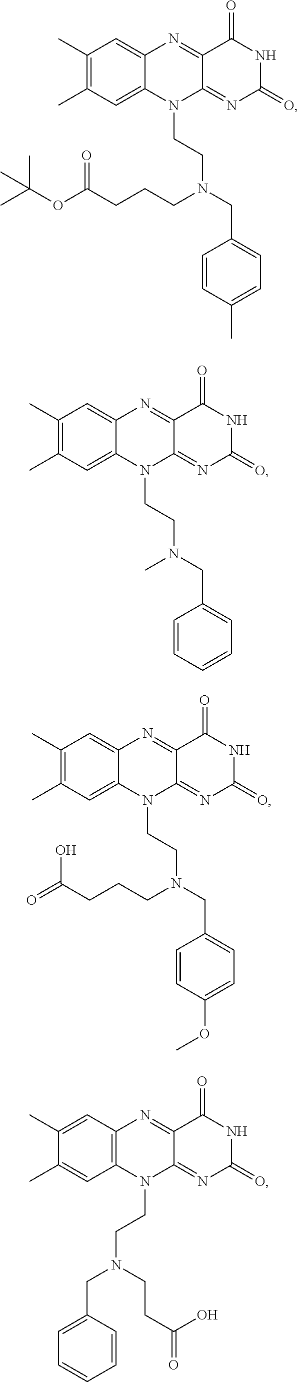 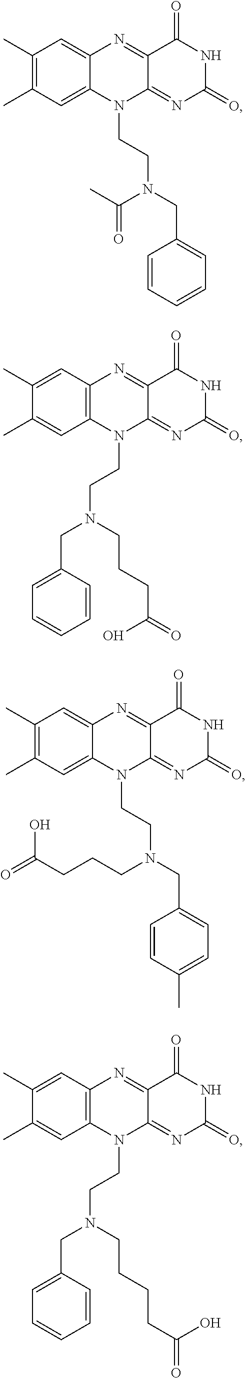 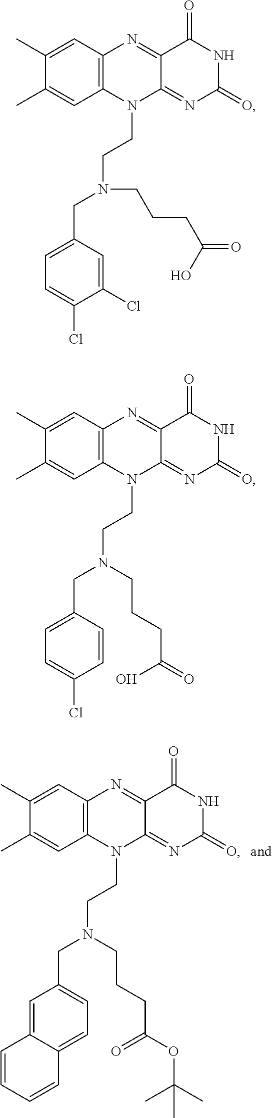 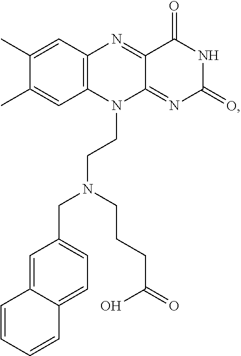
in free, pharmaceutically acceptable salt or prodrug form.

23. The method according to claim 1, wherein the Compound of Formula I further provides a proviso that when R₃ is 5-dihydrogen phosphate (2R,3S,4S)-trihydroxypentyl and R₁ is methyl, then R₂ is not dimethylamino.

24. (canceled)

25. (canceled)

26. (canceled)

27. (canceled)

28. The compound according to claim 30, wherein said compound is a compound of Formula III, 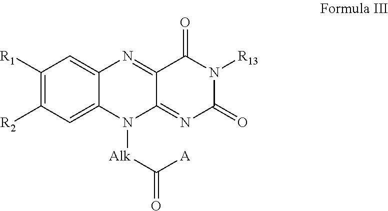
wherein:
(i) Alk is C₁₋₈ alkyl;
(ii) A is —OR₉ or —N(R₁₄)(R₁₅);
(iii) R₉ is H, —C₁, haloC₁₋₈alkyl, —C₁₋₄alkyl-OC(O)R₁₂, —C₁₋₄alkyl-O—C₁₋₄alkyl —C₁₋₄-alkyl-C(O)-(morphylin-4-yl), —C₃₋₇cycloalkyl, C₃₋₇cycloalkyl-C₁₋₄alkyl wherein the cycloalkyl is optionally substituted with hydroxy group; aryl or aryl-C₁₋₄alkyl, wherein said aryl is optionally substituted with one or more halo or C₁₋₄alkoxy;
(iv) R₁ is H, C₁₋₈ alkyl;
(v) R₂ is H, halo, —O—C₃₋₇cycloalkyl, O—C_{O-7} alkylC₃₋₇cycloalkyl, —N(R₄)(R₅), —(CH₂)—N(R₄)(R₅), —C₀₋₄alkyl-C₃₋₇cycloalkyl, heteroC₃₋₇cycloalkyl, 1-cyclopropyl-6-fluoro-7-[4-piperazin-1-yl]-4-oxo-quinoline-3-carboxylic acid, C₁₋₈alkyl or O—C₁₋₈alkyl, wherein the alkyl group is optionally substituted with one or more halo or hydroxy groups;
(vi) R₄ and R₅ are independently
a. H,
b. —C₀₋₄alkyl-C₃₋₇cycloalkyl,
c. heteroC₃₋₇cycloalkyl,
d. aryl,
e. aryl-C₁₋₈alkyl wherein the aryl is optionally substituted with halo,
f. —(CH₂)₃—N(H)—(CH₂)₄—N(H)—(CH₂)₃—N(H)₂,
g. —C₁₋₈alkyl wherein said alkyl is optionally substituted with one or more hydroxy groups;
(vii) R₁₂ is C₁₋₈alkyl or —OC₁₋₈alkyl; and
(viii) R₁₃ is H or C₁₋₄alkyl;
(ix) R₁₄ and R₁₅ are independently H, —OH, —S(O)₂CH₃, —OBn or —C₁₋₄alkyl,
in free, salt or prodrug form.

29. The compound according to claim 30, wherein said compound is a compound of Formula IV: 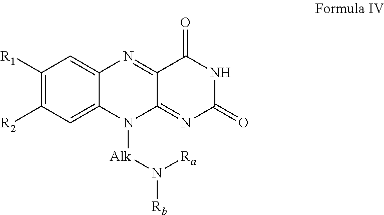
wherein:
(i) Alk is C₁₋₈alkyl;
(ii) Rₐ and R_{b} are independently H, —C₁₋₄alkyl, —(CH₂)₃C(NH₂)(COOH)CHF₂, —(CH₂)₃N(H)C(═NH)NH₂, —(CH₂)₅NH₂, —(CH₂)₂C(H)(OH)COOH, —C(O)(CH₂)₂COOH, —C₁₋₄alkyl-C(O)OR₉ (e.g., —CH₂CH₂CH₂CH₂C(O)OR₉, —CH₂CH₂CH₂C(O)OR₉, —CH₂CH₂C(O)OR₉ or —CH₂C(O)OR₉, —C(CH₃)(CH₃)C(O)OR₉), —C(O)CH₃, aryl, C(O)-aryl, aryl-C₁₋₄alkyl, heteroaryl, heteroaryl-C₁₋₄alkyl, wherein said aryl and heteroaryl groups are optionally substituted with one or more groups selected from —C(O)OR₉, —NH₂, —S(O)₂NH₂, —CH₂NH₂, halo, C₁₋₄alkoxy, C₁₋₄alkyl;
(iii) R₁ is H, C₁₋₈ alkyl;
(iv) R₂ is H, halo, O—C₃₋₇cycloalkyl, —N(R₄)(R₅), C₃₋₇cycloalkyl, C₁₋₈alkyl or —O—C₁₋₈alkyl wherein the alkyl group is optionally substituted with one or more halo or hydroxyl groups;
(v) R₄ and R₅ are independently H, C₃₋₇cycloalkyl, C₁₋₈alkyl wherein said alkyl is optionally substituted with one or more hydroxy groups;
(vi) R₉ is H or C₁₋₄alkyl;
(vii) R₁₂ is C₁₋₈alkyl or —OC₁₋₈alkyl,
in free, salt or prodrug form.

30. A compound selected from:
a) a Compound of formula I(i): 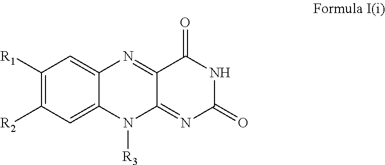
wherein
(i) R₁ is H, C₁₋₈ alkyl or C₃₋₇ cycloalkyl;
(ii) R₂ is H, halo, C₁₋₈alkyl, C₁₋₈alkoxy, —N(R₄)(R₅), C₃₋₇cycloalkyl or C₄₋₇heterocycle wherein said heterocycle is optionally substituted with C₁₋₈alkyl or hydroxyC₁₋₈alkyl; or
(iii) R₁ and R₂ are connected so as to form a cyclic ring structure optionally containing one or more heteroatoms selected from N, O and;
(iv) R₃ is H or C₁₋₈ alkyl, wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇);
(v) R₄ and R₅ are independently selected from H, C₃₋₇ cycloalkyl, C₄₋₇heterocycle, and C₁₋₈alkyl wherein said alkyl is optionally substituted with one or more groups selected from —OR₁₁, —C(O)OR₉, —N(R₆)(R₇), C₁₋₈alkoxyl, C₆₋₁₀aryl, C₅₋₁₀ heteroaryl wherein said aryl or heteroaryl are optionally substituted with halo, and C₄₋₇heterocycle wherein said heterocycle is optionally substituted with C₁₋₈alkyl;
(vi) R₆ and R₇ are independently selected from H, C₁₋₈alkyl, —OR₁₁, —C(O)OR₉, —C₁₋₈alkyl-C(O)OR₉, —C₁₋₈alkyl(amine)-C(O)OR₉, —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-C₁₋₈alkyl and aryl wherein said aryl and alkyl are optionally substituted with —COOR₉;
(vii) R₈ is H, C₁₋₈alkyl, —OR₁₁ or —OBn;
(viii) R₉ and R₁₇ are independently selected from H, C₁₋₈alkyl, —C₁₋₄alkyl-OC(O)R₁₂, phenyl and Bn wherein said phenyl and Bn are optionally substituted with one or more halo or C₁₋₄alkoxy;
(ix) R₁₀ is H, C₁₋₈alkyl, —C₁₋₈alkyl-OR₁₁, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl, or aryl wherein said aryl and alkyl are optionally substituted with —COOR₉, or —C₁₋₄alkyl-OC(O)R_{12;}
(x) R₁₁ is H, or —C₁₋₄alkyl-OC(O)R₁₂;
(xi) R₁₂ is C₁₋₈alkyl or —OC₁₋₈alkyl;
(xii) R₁₃, R₁₄, R₁₅ and R₁₆ are independently selected from H, C₁₋₈alkyl, and —C₁₋₈alkyl-COOR₁₈, wherein the alkyl group of C₁₋₈alkyl-COOR₁₈ is optionally substituted with hydroxyC₁₋₈alkyl, carboxyC₁₋₈alkyl;
(xiii) R₁₈ is H or C₁₋₈alkyl;
in free, salt or prodrug form,
b) a Compound of formula I: 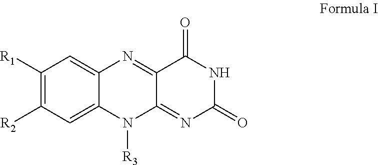
wherein
(i) R₁ is H, C₁₋₈ alkyl or C₃₋₇ cycloalkyl;
(ii) R₂ is H, halo, C₁₋₈alkyl, C₁₋₈alkoxy, —N(R₄)(R₅), C₃₋₇cycloalkyl or C₄₋₇heterocycle wherein said heterocycle is optionally substituted with C₁₋₈alkyl or hydroxyC₁₋₈alkyl;
(iii) R₃ is H or C₁₋₈ alkyl, wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇); or
(iv) R₁ and R₂ are connected so as to form a cyclic ring structure optionally containing one or more heteroatoms selected from N, O and S;
(v) R₄ and R₅ are independently selected from H, C₃₋₇ cycloalkyl, C₄₋₇heterocycle, and C₁₋₈alkyl wherein said alkyl is optionally substituted with one or more groups selected from —OR₁₁, —C(O)OR₉, —N(R₆)(R₇) (e.g., amino or dimethylamino), C₁₋₈alkoxyl, C₆₋₁₀aryl, C₅₋₁₀ heteroaryl wherein said aryl or heteroaryl are optionally substituted with halo, and C₄₋₇heterocycle wherein said heterocycle is optionally substituted with C₁₋₈alkyl;
(vi) R₆ and R₇ are independently selected from H, C₁₋₈alkyl, —C₁₋₈alkyl-OR₁₁, —C(O)OR₉, —C₁₋₈alkyl-C(O)OR₉, —C₁₋₈alkyl(amine)-C(O)OR₉ (e.g., —CH₂CH₂CH(NH₂)COOH), —C₁₋₈alkyl-C(O)N(H)R₈, —C₁₋₈alkyl-P(O)(OR₉)(OR₁₇)₅—C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄) —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl and aryl wherein said aryl is optionally substituted with —COOR₉;
(vii) R₈ is H, C₁₋₈alkyl, —OR₁₁ or —OBn;
(viii) R₉ and R₁₇ are independently selected from H, C₁₋₈alkyl, —C₁₋₄alkyl-OC(O)R₁₂, phenyl and Bn wherein said phenyl and Bn are optionally substituted with one or more halo or C₁₋₄alkoxy;
(ix) R₁₀ is H, C₁₋₈alkyl, —C₁₋₈alkyl-OR₁₁, —C₁₋₈alkyl-C(O)OR₉, —C₁₋₈alkyl(amine)-C(O)OR₉, —C₁₋₈alkyl-C(O)N(H)R₈, —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-ethyl, or aryl wherein said aryl is optionally substituted with —COOR₉, or —C₁₋₄alkyl-OC(O)R₁₂;
(x) R₁₁ is H, or —C₁₋₄alkyl-OC(O)R₁₂;
(xi) R₁₂ is C₁₋₈alkyl or —OC₁₋₈alkyl;
(xii) R₁₃, R₁₄, R₁₅ and R₁₆ are independently selected from H, C₁₋₈alkyl, and —C₁₋₈alkyl-COOR₁₈, wherein the alkyl group of C₁₋₈alkyl-COOR₁₈ is optionally substituted with hydroxyC₁₋₈alkyl, carboxyC₁₋₈alkyl; and
(xiii) R₁₈ is H or C₁₋₈alkyl;
in free, salt or prodrug form.
c) a Compound of formula I(ii): 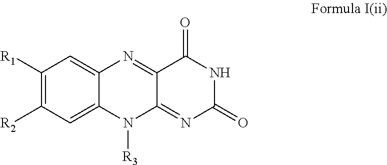
wherein
(i) R₁ is H, C₁₋₈ alkyl;
(ii) R₂ is H, halo, C₁₋₈alkyl, C₁₋₈alkoxy, —N(R₄)(R₅);
(iii) R₃ is C₁₋₈ alkyl, wherein the alkyl group is optionally substituted with one or more groups selected from —OP(O)(OR₉)(OR₁₇), —OP(O)(OR₉)(NR₁₃R₁₄), —OP(O)(NR₁₃R₁₄)(NR₁₅R₁₆), —P(O)(OR₉)(OR₁₇), —P(O)(OR₉)(NR₁₃R₁₄), —P(O)(NR₁₃R₁₄)(NR₁₅R₁₆), —CN, —C(O)OR₉, —C(O)N(H)(R₈), —OR₁₀, —C(O)N(R₆)(R₇), and —N(R₆)(R₇);
(iv) R₄ and R₅ are independently selected from H, C₃₋₇ cycloalkyl, and C₁₋₈alkyl wherein said alkyl is optionally substituted with one or more groups selected from —OR₁₁;
(v) R₆ and R₇ are independently selected from H, —C₁₋₈alkyl, —C₁₋₈alkyl-C(O)OR₉—C₁₋₈alkyl(amine)-C(O)OR₉, —C₁₋₈alkyl-C(O)N(H)R₈, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄)—C₁₋₈alkyl-OP(O)(NR₁₃R₁₄)(NR₁₅R₁₆), 7,8-dimethyl-isoalloxazin-10-yl-C₁₋₈alkyl and aryl wherein said aryl and alkyl are optionally substituted with —COOR₉;
(vi) R₈ is H, C₁₋₈alkyl;
(vii) R₉ and R₁₇ are independently selected from H, C₁₋₈alkyl, —C₁₋₄alkyl-OC(O)R₁₂, phenyl and Bn wherein said phenyl and Bn are optionally substituted with one or more halo or C₁₋₄alkoxy;
(viii) R₁₀ is —C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), —C₁₋₈alkyl-OP(O)(OR₉)(OR₁₇), —C₁₋₈alkyl-OP(O)(OR₉)(NR₁₃R₁₄), —C₁₋₈alkyl-OP(O)(NR₁₃R₁₄)(NR₁₅R₁₆), —C₁₋₈alkyl-N(H)—S(O)₂(CF₃), 7,8-dimethyl-isoalloxazin-10-yl-C₁₋₈alkyl, or aryl wherein said aryl and alkyl are optionally substituted with —COOR₉, or —C₁₋₄alkyl-OC(O)R₁₂;
(ix) R₁₁ is H, or —C₁₋₄alkyl-OC(O)R₁₂;
(x) R₁₂ is C₁₋₈alkyl or —OC₁₋₈alkyl;
(xi) R₁₃, R₁₄, R₁₅ and R₁₆ are independently selected from H, C₁₋₈alkyl, and —C₁₋₈alkyl-COOR₁₈, wherein the alkyl group of C₁₋₈alkyl-COOR₁₈ is optionally substituted with hydroxyC₁₋₈alkyl, carboxyC₁₋₈alkyl; and
(xii) R₁₈ is H or C₁₋₈alkyl;
in free, salt or prodrug form;
d) a Compound of formula I(iii): 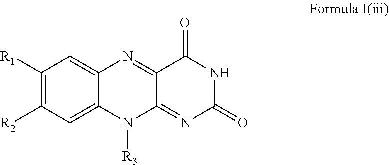
wherein
(i) R₁ is H, C₁₋₈ alkyl;
(ii) R₂ is H, halo, C₁₋₈alkyl, C₁₋₈alkoxy, —N(R₄)(R₅);
(iii) R₃ is C₁₋₈ alkyl, wherein the alkyl group is optionally substituted with one or more groups selected from —P(O)(OR₉)(OR₁₇), —P(O)(OR₉)(NR₁₃R₁₄), —P(O)(NR₁₃R₁₄)(NR₁₅R₁₆), —C(O)OR₉, —OR₁₀, —C(O)N(R₆)(R₇), and —N(R₆)(R₇);
(iv) R₄ and R₅ are independently selected from H, C₃₋₇ cycloalkyl, and C₁₋₈alkyl wherein said alkyl is optionally substituted with one or more groups selected from —OH;
(v) R₆ and R₇ are independently selected from H, —C₁₋₈alkyl, —C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl, wherein said aryl and alkyl are optionally substituted with —COOR₉;
(vi) R₉ and R₁₇ are independently selected from H, C₁₋₈alkyl, —C₁₋₄alkyl-OC(O)R₁₂;
(vii) R₁₀ is H, —C₁₋₈ alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆);
(viii) R₁₂ is C₁₋₈alkyl;
(ix) R₁₃, R₁₄, R₁₅ and R₁₆ are independently selected from H, C₁₋₈alkyl, and —C₁₋₈alkyl-COOR₁₈;
(x) R₁₈ is H or C₁₋₈alkyl;
in free, salt or prodrug form;
e) a Compound of formula I(iv): 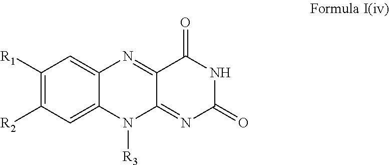
wherein
(i) R₁ is H, C₁₋₈ alkyl;
(ii) R₂ is H, halo, C₁₋₈alkyl, C₁₋₈alkoxy, —N(R₄)(R₅);
(iii) R₃ is C₁₋₈alkyl-N(R₆)(R₇), C₁₋₈alkyl-C(O)N(R₆)(R₇), C₁₋₈alkyl-P(O)(OR₉)(OR₁₇), C₁₋₈alkyl-P(O)(OR₉)(NR₁₃R₁₄), C₁₋₈alkyl-P(O)(NR₁₃R₁₄)(NR₁₅R₁₆), C₁₋₈alkyl-C(O)OR₉, C₁₋₈alkyl-OR₁₀;
(iv) R₄ and R₅ are independently selected from H, C₃₋₇ cycloalkyl, and C₁₋₈alkyl wherein said alkyl is optionally substituted with one or more groups selected from —OR₁₁;
(v) R₆ and R₇ are independently selected from H, C₁₋₈alkyl, —C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl and aryl wherein said aryl and alkyl are optionally substituted with —COOR₉;
(vi) R₉ and R₁₇ are independently selected from H, C₁₋₈alkyl, —C₁₋₄alkyl-OC(O)R_{12;}
(vii) R₁₀ is H, —C₁₋₈alkyl-C(O)OR₉, —C₁₋₈alkyl(amine)-C(O)OR₉ (e.g., —CH₂CH₂CH(NH₂)COOH), —C₁₋₈alkyl-P(O)(OR₉)(OR₁₇), —C₁₋₈alkyl-P(O) (OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl wherein said aryl and alkyl are optionally substituted with —COOR₉;
(viii) R₁₂ is C₁₋₈alkyl;
(ix) R₁₃, R₁₄, R₁₅ and R₁₆ are independently selected from H, C₁₋₈alkyl, and —C₁₋₈alkyl-COOR₁₈;
(x) R₁₈ is H or C₁₋₈alkyl;
in free, salt or prodrug form;
f) a Compound of formula I(v): 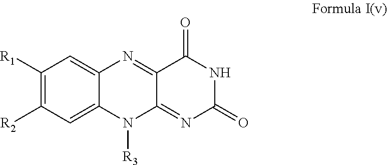
wherein
(i) R₁ is H, C₁₋₈ alkyl;
(ii) R₂ is H, halo, C₁₋₈alkyl, C₁₋₈alkoxy, —N(R₄)(R₅);
(iii) R₃ is C₂alkyl-N(R₆)(R₇), C₁₋₂alkyl-C(O)N(R₆)(R₇), C₁₋₈alkyl-P(O)(OR₉)(OR₁₇), C₁₋₈alkyl-P(O)(OR₉)(NR₁₃R₁₄), C₁₋₈alkyl-P(O)(NR₁₃R₁₄)(NR₁₅R₁₆), C₁₋₈alkyl-C(O)OR₉, C₁₋₈alkyl-OR₁₀;
(iv) R₄ and R₅ are independently selected from H, C₃₋₇ cycloalkyl, and C₁₋₈alkyl wherein said alkyl is optionally substituted with one or more groups selected from —OR₁₁;
(v) R₆ and R₇ are independently selected from —C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl and aryl wherein said aryl and alkyl are optionally substituted with —COOR₉;
(vi) R₉ and R₁₇ are independently selected from H, C₁₋₈alkyl, —C₁₋₄alkyl-OC(O)R_{12;}
(vii) R₁₀ is H, —C₁₋₈alkyl-C(O)OR₉, —C₁₋₈alkyl(amine)-C(O)OR₉, —C₁₋₈alkyl-P(O)(OR₉)(OR₁₇), —C₁₋₈alkyl-P(O)(OR₉)(NR₁₃R₁₄), —C₁₋₈alkyl-P(O)(NR₁₃R₁₄)(NR₁₅R₁₆), 7,8-dimethyl-isoalloxazin-10-yl-C₁₋₈alkyl, or aryl wherein said aryl and alkyl are optionally substituted with —COOR₉;
(viii) R₁₂ is C₁₋₈alkyl;
(ix) R₁₃, R₁₀, R₁₅ and R₁₆ are independently selected from H, C₁₋₈alkyl, and —C₁₋₈alkyl-COOR₁₈;
(x) R₁₈ is H or C₁₋₈alkyl;
in free, salt or prodrug form;
g) a Compound of formula III: 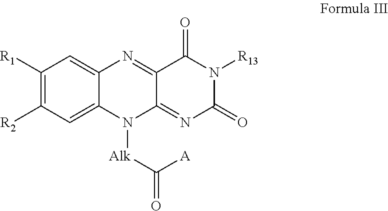
wherein:
(i) Alk is C₁₋₈ alkyl;
(ii) A is —OR₉ or —N(R₁₄)(R₁₅);
(iii) R₉ is H, —C₁₋₈alkyl, -haloC₁₋₈alkyl, —C₁₋₄alkyl-OC(O)R₁₂, —C₁₋₄alkyl-O—C₁₋₄alkyl, —C₁₋₄alkyl-C(O)-(morphylin-4-yl), —C₃₋₇cycloalkyl, C₃₋₇cycloalkyl-C₁₋₄alkyl wherein the cycloalkyl is optionally substituted with hydroxy group; aryl or aryl-C₁₋₄alkyl, wherein said aryl is optionally substituted with one or more halo or C₁₋₄alkoxy;
(iv) R₁ is H, C₁₋₈ alkyl;
(v) R₂ is H, halo, —O—C₃₋₇cycloalkyl, —O—C₀₋₇ alkylC₃₋₇cycloalkyl, —N(R₄)(R₅), —(CH₂)—NR₄)(R₅), —C₀₋₄alkyl-C₃₋₇cycloalkyl, heteroC₃₋₇cycloalkyl, 1-cyclopropyl-6-fluoro-7-[4-piperazin-1-yl]-4-oxo-quinoline-3-carboxylic acid), C₁₋₈alkyl or —O—C₁₋₈alkyl, wherein the alkyl group is optionally substituted with one or more halo or hydroxy groups;
(vi) R₄ and R₅ are independently
a. H,
b. —C₀₋₄alkyl-C₃₋₇cycloalkyl,
c. heteroC₃₋₇cycloalkyl,
d. aryl,
e. aryl-C₁₋₈alkyl wherein the aryl is optionally substituted with halo,
f. —(CH₂)₃—N(H)—(CH₂)₄—N(H)—(CH₂)₃—N(H)₂,
g. —C₁₋₈alkyl wherein said alkyl is optionally substituted with one or more hydroxy groups;
(vii) R₁₂ is C₁₋₈alkyl or —OC₁₋₈alkyl; and
(viii) R₁₃ is H or C₁₋₄alkyl;
(ix) R₁₄ and R₁₅ are independently H, —OH, —S(O)₂CH₃, —OBn or —C₁₋₄alkyl,
in free, salt or prodrug form.
h) a Compound of formula IV: 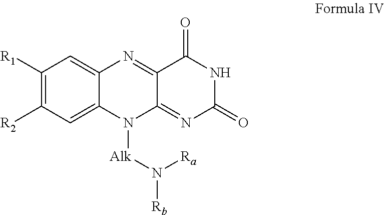
wherein:
(viii) Alk is C₁₋₈ alkyl;
(ix) Rₐ and R_{b} are independently H, —C₁₋₄alkyl, —(CH₂)₃C(NH₂)(COOH)CHF₂, —(CH₂)₃N(H)C(═NH)NH₂, —(CH₂)₅NH₂, —(CH₂)₂C(H)(OH)COOH, —C(O)(CH₂)₂COOH, —C₁₋₄alkyl-C(O)OR₉ (e.g., —CH₂CH₂CH₂CH₂C(O)OR₉, —CH₂CH₂CH₂C(O)OR₉, —CH₂CH₂C(O)OR₉ or —CH₂C(O)OR₉, —C(CH₃)(CH₃)C(O)OR₉), —C(O)CH₃, aryl, —C(O)-aryl, aryl-C₁₋₄alkyl, heteroaryl, heteroaryl-C₁₋₄alkyl, wherein said aryl and heteroaryl groups are optionally substituted with one or more groups selected from —C(O)OR₉, —NH₂, —S(O)₂NH₂, —CH₂NH₂, halo, C₁₋₄alkoxy, C₁₋₄alkyl;
(x) R₁ is H, C₁₋₈ alkyl;
(xi) R₂ is H, halo, —O—C₃₋₇cycloalkyl, —N(R₄)(R₅), C₃₋₇cycloalkyl, C₁₋₈alkyl or —O—C₁₋₈alkyl wherein the alkyl group is optionally substituted with one or more halo or hydroxyl groups;
(xii) R₄ and R₅ are independently H, C₃₋₇cycloalkyl, C₁₋₈alkyl wherein said alkyl is optionally substituted with one or more hydroxy groups;
(xiii) R₉ is H or C₁₋₄alkyl;
(xiv) R₁₂ is C₁₋₈alkyl or —OC₁₋₈alkyl,
in free, salt or prodrug form;
i) a Compound of formula V: 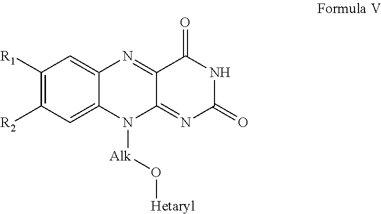
wherein Alk is C₁₋₆alkyl and hetaryl is heteroaryl and R₁ and R₂ are independently H, C₁₋₄alkyl, in free, pharmaceutically acceptable salt or prodrug form;
and
j) a Compound of formula VI: 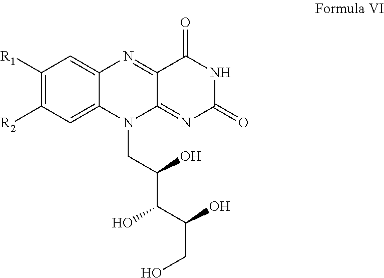
wherein R₁ is H or C₁₋₄alkyl and R₂ is cyano,
provided that:
(a) when R₁ is methyl and R₂ is chloro, then R₃ is not methyl;
(b) when R₁ is H and R₂ is dimethylamine, then R₃ is not H;
(c) when R₃ is (2R,3S,4S)-2,3,4,5-tetrahydroxypentyl or 5-dihydrogen phosphate
(2R,3S,4S)-trihydroxypentyl, and R₁ is methyl, then R₂ is not methyl;
(d) when R₃ is (2R,3S,4S)-2,3,4,5-tetrahydroxypentyl and R₁ is methyl, then R₂ is not dimethylamino;
(e) when R₁ is methyl and R₂ is alkoxy, then R₃ is not 2,3,4,5-tetrahydroxypentyl;
(f) when R₁ and R₂ are independently selected from a group consisting of C₁₋₅ alkyl, C₁₋₅ alkoxy, amino, hydrogen and halogen group, R₃ is not —(CH₂)₂₋₆-phosphate.
(g) when R₃ is 5-dihydrogen phosphate (2R,3S,4S)-trihydroxypentyl and R₁ is methyl, then R₂ is not dimethylamino;
(h) when R₃ is —(CH₂)₂₋₆-phosphate or —(CH₂)₂₋₆-(sodium phosphate), then R₁ and R₂ are not C₁₋₅alkyl, C₁₋₅alkoxy, amino, hydrogen or halogen group;
(i) when R₁ is H or C₁₋₆alkyl, and R₂ is hydrogen, halo, C₁₋₆alkyl, C₁₋₆alkoxy, dialkylamino or —NHCH₂CH(OH)CH(OH)CH(OH)CH₂OH, then R₃ is not H, CH₂CH₂CH(OH)CH(OH)CH₂OH, —CH₂CH₂OH, CH₂CH(OH)CH(OH)CH(OH)—CH₂OH, CH₂CH(OH)CH(OH)CH(OH)CH(OH)CH₃ or CH₂CH(OH)CH(OH)—CH(OH)CH₂OPO₃;
(j) when R₁ and R₂ are both H, R₃ is not —(CH₂)₀₋₂CH₂—N(R′)₂ or —(CH₂)₀₋₂CH₂—N⁺(R′)₃—X⁻ wherein R′ is H or alkyl and X is Cl⁻, F⁻, oxalate, methosulfate, Br⁻;
(k) when R₁ is H and R₂ is chloro, then R₃ is not -alkyl-N(R^{a})(R^{b}) wherein R^{a} is alkyl and R^{b} is hydroxyalkyl;
(l) when R₁ and R₂ and are selected from H, amine, polyamine, halogen, saccharide or C₁₋₇ alkyl wherein the C atoms of the alkyl group may be replaced with N or O, wherein said alkyl group may be substituted with halogen, OH, NH₂, COOH, OR^{d}, NR^{d}R^{e}, CONR^{d}R^{e}, wherein R^{d} and R^{e} are independently alkyl, and aryl group, then R₃ is not H, amine, polyamine, halogen, saccharide or C₁₋₇ alkyl wherein the C atoms of the alkyl group may be replaced with N or O, wherein said alkyl group may be substituted with halogen, OH, NH₂, COOH, OR^{d}, NR^{d}R^{e}, CONR^{d}R^{e}, wherein R^{d} and R^{e} are independently alkyl, or aryl group;
(m) when R₁ is methyl and R₂ is —N(H)CH₃, then R₃ is not —CH₂—(CHOH)₃—CH₂OH;
(n) when R₁ and R₂ are both ethyl, then R₃ is not —CH₂—(CHOH)₃—CH₂OH;
(o) when R₁ and R₂ are methyl, then R₃ is not H, alkyl, polysaccharide or an alkyl etherified or acylated glycoside of polysaccharide;
(p) when R₃ is H, C₁₋₇alkyl, C₁₋₇haloalkyl, C₁₋₇hydroxyalkyl, C₁₋₇-aminoalkyl, C₁₋₇ carboxyalkyl, or C₅₋₂₀arylC₁₋₇alkyl, then R₁ and R₂ are not H, halo, or C₁₋₇alkyl optionally substituted with —OH, halo, —COOH, —N(R_{f})(R_{g}), or —C(O)N(R_{f})(R_{g}), wherein R_{f} and R_{g} are independently H, C₁₋₇alkyl, C₃₋₂₀heterocycle, C₅₋₂₀aryl;
(q) when R₁ and R₂ are H or lower alkyl, the R₃ is not lower alkyl; and
the Compound is not riboflavin 5′-(hydrogensulfate), 7,8-dimethyl-10-(D-allityl) isoalloxazine.

31. The Compound according to claim 30 selected from: 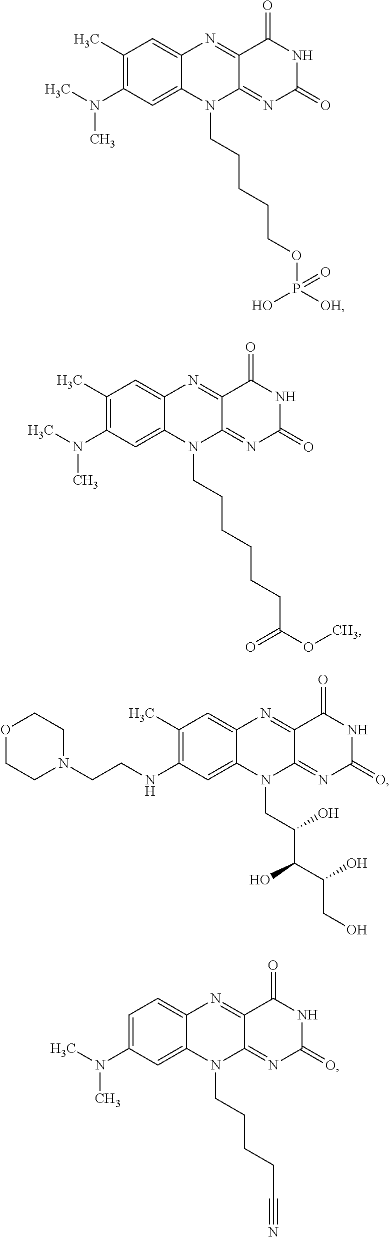 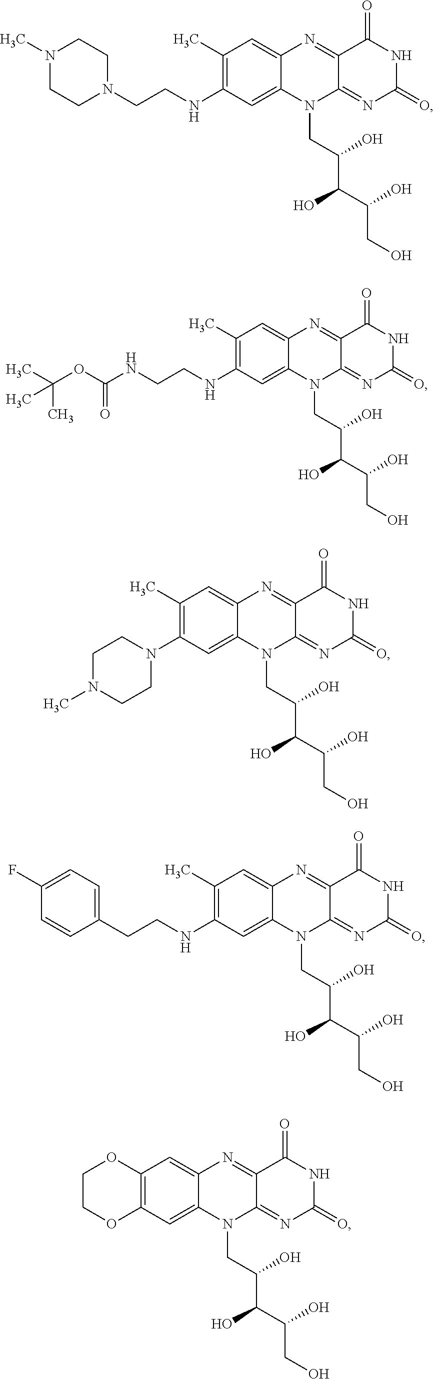 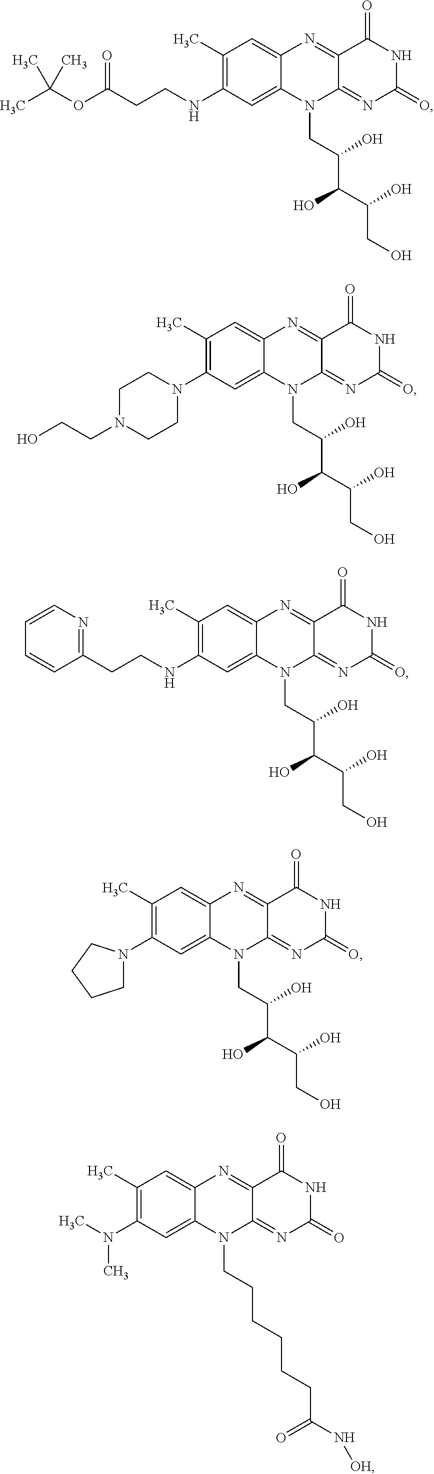 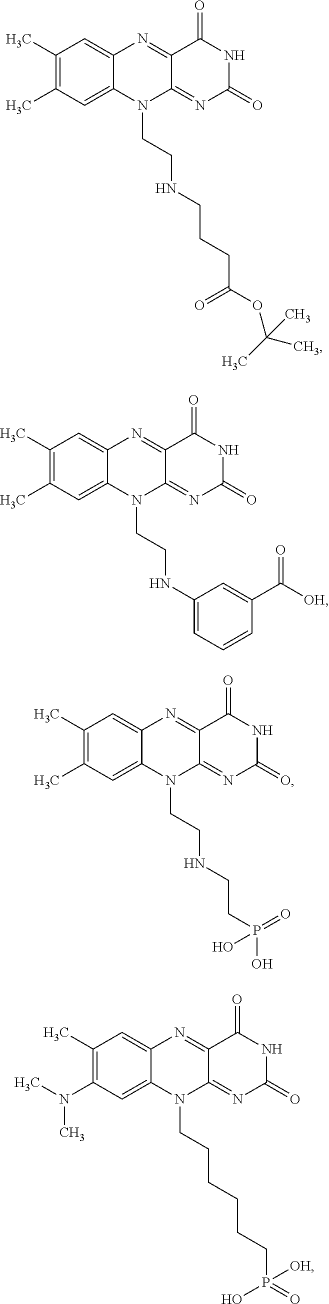 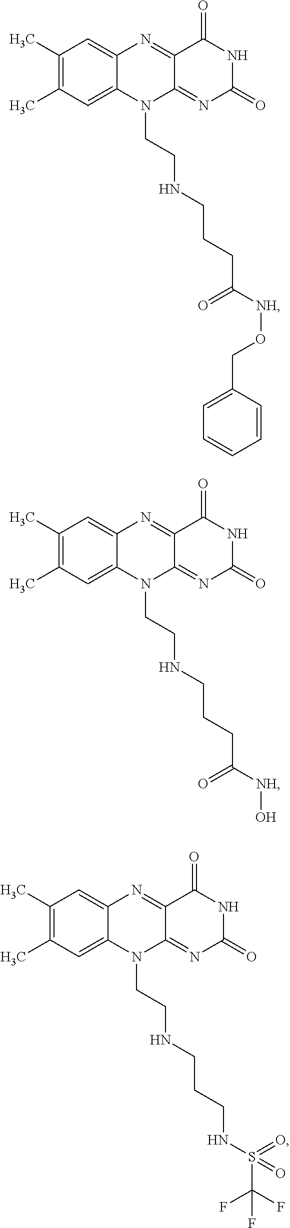 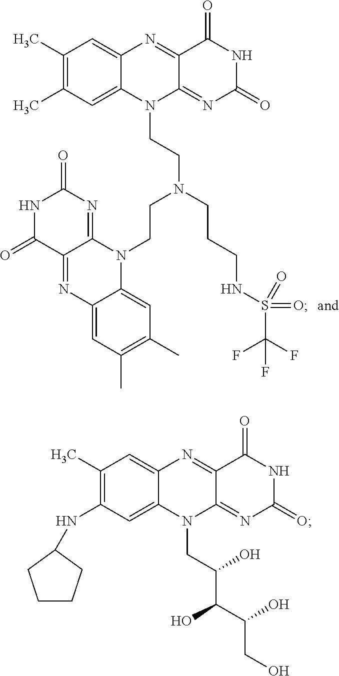
in free, salt or prodrug form.
